(19) United States
(12) Patent Application Publication
(10) Pub. No.: US 2018/0282402 A1
(43) Pub. Date: Oct. 4, 2018

(54) ANTI-HRS ANTIBODIES AND COMBINATON THERAPIES FOR TREATING CANCERS

(71) Applicant: aTyr Pharma, Inc., San Diego, CA (US)

(72) Inventors: Ryan Andrew Adams, San Diego, CA (US); Luke Burman, San Diego, CA (US); Yeeting Chong, San Diego, CA (US); David King, Encinitas, CA (US); John D. Mendlein, Encinitas, CA (US); Leslie Nangle Greene, San Diego, CA (US); Kathleen Ogilvie, San Diego, CA (US); Kaitlyn Rauch, San Diego, CA (US)

(21) Appl. No.: 15/828,170

(22) Filed: Nov. 30, 2017

Related U.S. Application Data
(60) Provisional application No. 62/581,431, filed on Nov. 3, 2017.
(60) Provisional application No. 62/566,995, filed on Oct. 2, 2017.
(60) Provisional application No. 62/516,456, filed on Jun. 7, 2017.
(60) Provisional application No. 62/481,918, filed on Apr. 5, 2017.
(60) Provisional application No. 62/466,800, filed on Mar. 3, 2017.
(60) Provisional application No. 62/428,307, filed on Nov. 30, 2016.

(51) Int. Cl. C07K 16/18; A61K 45/06; A61K 39/395; A61P 35/04

(52) U.S. Cl. CPC C07K 16/18; A61K 45/06; A61K 2039/572; A61K 39/3955; A61P 35/04; A61K 39/39541

(57) ABSTRACT

Provided are antibodies that specifically bind to human histidyl-tRNA synthetase and related therapeutic compositions and methods for treating cancer, including as standalone therapies or in combination with cancer immunotherapies, for example, immune checkpoint modulators such as PD-1 inhibitors.

### CROSS-REFERENCE TO RELATED APPLICATIONS

This application claims priority under 35 U.S.C. 119(e) to U.S. Application No. 62/428,307, filed Nov. 30, 2016; U.S. Application No. 62/466,800, filed Mar. 3, 2017; U.S. Application No. 62/481,918, filed Apr. 5, 2017; U.S. Application No. 62/516,456, filed Jun. 7, 2017; U.S. Application No. 62/566,995, filed Oct. 2, 2017; and U.S. Application No. 62/581,431, filed Nov. 3, 2017, each of which is incorporated by reference in its entirety.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is ATYR_127_06US_ST25.txt. The text file is 556 KB, was created on Nov. 30, 2017, and is being submitted electronically via EFS-Web.

### BACKGROUND

### Technical Field

Embodiments of the present disclosure relate to antibodies that specifically bind to human histidyl-tRNA synthetase (HRS) polypeptides and related therapeutic compositions and methods for treating cancers, including as standalone therapeutics or in combination with cancer immunotherapies, for example, immune checkpoint modulators such as PD-1 inhibitors.

### BRIEF SUMMARY

Embodiments of the present disclosure include therapeutic compositions, comprising at least one antibody or antigen-binding fragment thereof that specifically binds to a human histidyl-tRNA synthetase (HRS) polypeptide (an anti-HRS antibody).

In some embodiments, the at least one antibody or antigen-binding fragment thereof specifically binds to the full-length HRS polypeptide (SEQ ID NO: 1), optionally with an affinity of about 10 pM to about 500 pM or to about 1 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, or 1 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, or about 100 pM to about 1 nM.

In some embodiments, the at least one antibody or antigen-binding fragment thereof specifically binds to a human HRS polypeptide selected from Table H1, optionally with an affinity of about 10 pM to about 500 pM or to about 1 nM, or about, at least about, or no more than about 10, 20, 30, 40, 50, 60, 70, 80, 90, 100, 110, 120, 130, 140, 150, 160, 170, 180, 190, 200, 300, 400, 500, 600, 700, 800, 900 pM, or 1 nM, or optionally with an affinity that ranges from about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, or about 100 pM to about 1 nM, and optionally wherein the at least one antibody or antigen-binding fragment thereof is cross-reactive with an HRS polypeptide selected from Table H2.

In some embodiments, the at least one antibody or antigen-binding fragment thereof has an affinity (Kd) for each of (i) a human HRS polypeptide and (ii) the corresponding region of a cynomolgus monkey HRS polypeptide, wherein the affinity for (i) and (ii) is within the range of about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, or about 100 pM to about 1 nM.

In some embodiments, the at least one antibody or antigen-binding fragment thereof specifically binds to at least one epitope within the N-terminal domain (about residues 1-100) of the human HRS polypeptide, optionally an epitope within the WHEP domain, optionally an epitope within about residues 1-100, 10-100, 20-100, 30-100, 40-100, 50-100, 60-100, 70-100, 80-100, 90-100, 1-90, 10-90, 20-90, 30-90, 40-90, 50-90, 60-90, 70-90, 80-90, 1-80, 10-80, 20-80, 30-80, 40-80, 50-80, 60-80, 70-80, 1-70, 10-70, 20-70, 30-70, 40-70, 50-70, 60-70, 1-60, 10-60, 20-60, 30-60, 40-60, 50-60, 1-50, 10-50, 20-50, 30-50, 40-50, 1-40, 10-40, 20-40, 30-40, 1-30, 10-30, 20-30, 1-20, 10-20, or 1-10 of SEQ ID NO:1 (FL human HRS).

In some embodiments, the at least one antibody or antigen-binding fragment thereof specifically binds to at least one epitope within the aminoacylation domain (about residues 61-398) of the human HRS polypeptide, optionally an epitope within about residues 61-398, 70-398, 80-398, 90-398, 100-398, 110-398, 120-398, 130-398, 140-398, 150-398, 160-398, 170-398, 180-398, 190-398, 200-398, 210-398, 220-398, 230-398, 240-398, 250-398, 260-398, 270-398, 280-398, 290-398, 300-398, 310-398, 320-398, 330-398, 340-398, 350-398, 360-398, 370-398, 380-398, or 60-388, 60-380, 60-370, 60-360, 60-350, 60-340, 60-330, 60-320, 60-310, 60-300, 60-290, 60-280, 60-270, 60-260, 60-250, 60-240, 60-230, 60-220, 60-210, 60-200, 60-180, 60-170, 60-160, 60-150, 60-140, 60-130, 60-120, 60-110, 60-100, 60-90, 60-80, or 60-70 of SEQ ID NO: 1 (FL human HRS).

In some embodiments, the at least one antibody or antigen-binding fragment thereof specifically binds to at least one epitope within the anticodon binding domain (about residues 399-506) of the human HRS polypeptide, optionally an epitope within about residues 399-500, 399-490, 399-480, 399-470, 399-460, 399-450, 399-440, 399-430, 399-420, 399-410, or 400-509, 410-509, 420-509, 430-509, 440-509, 450-509, 460-509, 470-509, 480-509, 490-509, or 500-509 of SEQ ID NO: 1 (FL human HRS).

In some embodiments, the at least one antibody or antigen-binding fragment thereof specifically binds to a single linear epitope within the N-terminal domain (˜residues 1-100) optionally within the WHEP domain (˜residues 3-43), a single linear epitope within the aminoacylation domain (˜residues 61-398), or a single linear epitope within the anticodon binding domain (residues 399-506) of the human HRS polypeptide.

In some embodiments, the at least one antibody or antigen-binding fragment thereof specifically binds to a conformational epitope composed of two or more discontinuous epitope regions. In some embodiments, the at least one antibody or antigen-binding fragment thereof specifically binds to a conformational epitope comprising or consisting of:

(a) a first epitope region within the N-terminal domain (˜residues 1-100) optionally within the WHEP domain (˜residues 1-60 or ˜residues 3-43), and second epitope region within the anticodon binding domain (˜residues 399-509 or ˜residues 406-501) of the human HRS polypeptide;

(b) a first epitope region within the N-terminal domain (˜residues 1-100) optionally within the WHEP domain (˜residues 1-60 or ˜residues 3-43), and a second epitope region within the aminoacylation domain (˜residues 54-398 or ˜residues 61-398) of the human HRS polypeptide; or

(c) a first epitope region within the N-terminal domain (residues 1-100) optionally within the WHEP domain (residues 1-60 or ˜residues 3-43), and second, different epitope region within the N-terminal domain (˜residues 1-100) optionally within the WHEP domain (˜residues 1-60 or ˜residues 3-43).

In some embodiments, the at least one antibody or antigen-binding fragment thereof interferes with binding of the human HRS polypeptide to a human neuropilin-2 (NP2) polypeptide. In some embodiments, the human NP2 polypeptide is selected from Table N1. In some embodiments, the at least one antibody or antigen-binding fragment thereof binds at least one epitope within a region of an HRS polypeptide that interacts with at least one neuropilin domain. In some embodiments, the at least one neuropilin domain is selected from one or more of the Neuropilin A1 domain, Neuropilin A2 domain, neuropilin B1 domain, neuropilin B2 domain, neuropilin C domain, neuropilin A1A2 combined domain, neuropilin B1B2 combined domain, neuropilin A2B1 combined domain, neuropilin A2B1B2 combined domain, neuropilin A2B1B2C combined domain, neuropilin A1A2B1 combined domain, neuropilin A1A2B1B2 combined domain, and the neuropilin A1A2B1B2C combined domain. In some embodiments, the at least one antibody or antigen-binding fragment thereof is a blocking antibody which inhibits about or at least about 80-100% of the theoretical maximal binding between the HRS polypeptide and the NP2 polypeptide after pre-incubation with the HRS polypeptide in a stoichiometrically equivalent amount, optionally about or at least about 80, 85, 90, 95, or 100% of the theoretical maximal binding. In some embodiments, the at least one antibody or antigen-binding fragment thereof is a partial-blocking antibody which inhibits about 20-80% of the theoretical maximal binding between the HRS polypeptide and the NP2 polypeptide after pre-incubation with the HRS polypeptide in a stoichiometrically equivalent amount, optionally about or at least about 20, 25, 30, 35, 40, 45, 50, 55, 60, 65, 70, 75, or 80% of the theoretical maximal binding. In some embodiments, the at least one antibody or antigen-binding fragment thereof is a non-blocking antibody which inhibits about or less than about 10% of the theoretical maximal binding between the HRS polypeptide and the NP2 polypeptide after pre-incubation with the HRS polypeptide in a stoichiometrically equivalent amount. In some embodiments, the at least one blocking antibody specifically binds to a splice variant selected from Table H1, optionally a HRS splice variant selected from SV9 (HRS(1-60)), SV11(HRS(1-60)+(399-509)) and SV14(HRS(1-100)+(399-509)). In some embodiments, the at least one blocking antibody specifically binds to a monomeric form of the HRS polypeptide, and substantially does not bind to a dimeric or multimeric form of the HRS polypeptide.

In some embodiments, the at least one antibody or antigen-binding fragment thereof increases the rate of clearance of an HRS polypeptide, or decreases the circulating levels of an HRS polypeptide, in the serum of a subject relative to pre-dosing levels of the HRS polypeptide, optionally by about or at least about 100, 200, 300, 400, or 500%.

In some embodiments, the at least one antibody or antigen-binding fragment thereof specifically binds to a corresponding epitope within a non-human HRS polypeptide selected from Table H2, wherein the binding affinities for the human and non-human HRS polypeptides are within about 1.2 fold, about 1.3 fold, about 1.4 fold, about 1.5 fold, about 1.6 fold, about 1.7 fold, about 1.8 fold, about 1.9 fold, about 2, about fold, 3, about 4 fold, about 5 fold, or about 10 fold.

In some embodiments, the at least one antibody or antigen-binding fragment thereof comprises an IgA (including subclasses IgA1 and IgA2), IgD, IgE, IgG (including subclasses IgG1, IgG2, IgG3, and IgG4), or IgM Fc domain, optionally a human Fc domain, or a hybrid and/or variant thereof. In some embodiments, the at least one antibody or antigen-binding fragment thereof comprises an IgG Fc domain with high effector function in humans, optionally an IgG1 or IgG3 Fc domain. In some embodiments, the at least one antibody or antigen-binding fragment thereof comprises an IgG Fc domain with low effector function in humans, optionally an IgG2 or IgG4 Fc domain. In some embodiments, the at least one antibody or antigen-binding fragment thereof comprises an IgG1 or IgG4 Fc domain, optionally selected from Table F1.

In some embodiments, the at least one antibody or antigen-binding fragment thereof comprises

a heavy chain variable region (V_{H}) sequence that comprises complementary determining region V_{H}CDR1, V_{H}CDR2, and V_{H}CDR3 sequences selected from Table A1 and variants thereof which specifically bind to the human HRS polypeptide; and

a light chain variable region (V_{L}) sequence that comprises complementary determining region V_{L}CDR1, V_{L}CDR2, and V_{L}CDR3 sequences selected from Table A1 and variants thereof which specifically bind to the human HRS polypeptide,

including affinity matured variants of the foregoing which specifically bind to the human HRS polypeptide.

In some embodiments:

the V_{H}CDR1, V_{H}CDR2, and V_{H}CDR3 sequences comprise the consensus sequences of SEQ ID NOs:396, 397, and 398 (as defined in Table A3), respectively, and the V_{L}CDR1, V_{L}CDR2, and V_{L}CDR3 sequences comprise the consensus sequences SEQ ID NOs: 399, 400, and 401 (as defined in Table A3), respectively, including variants thereof;

the V_{H}CDR1, V_{H}CDR2, and V_{H}CDR3 sequences comprise the consensus sequences of SEQ ID NOs: 402, 403, and 404 (as defined in Table A3), respectively, and the V_{L}CDR1, V_{L}CDR2, and V_{L}CDR3 sequences comprise the consensus sequences of SEQ ID NOs: 405, 406, and 407 (as defined in Table A3), respectively, including variants thereof;

the V_{H}CDR1, V_{H}CDR2, and V_{H}CDR3 sequences comprise the consensus sequences of SEQ ID NOs: 408, 409, and 410 (as defined in Table A3), respectively, and the V_{L}CDR1, V_{L}CDR2, and V_{L}CDR3 sequences comprise the consensus sequences of SEQ ID NOs: 411, 412, and 413 (as defined in Table A3), respectively, including variants thereof;

the V_{H}CDR1, V_{H}CDR2, and V_{H}CDR3 sequences comprise SEQ ID NOs: 12, 13, and 14, respectively, and the V_{L}CDR1, V_{L}CDR2, and V_{L}CDR3 sequences comprise SEQ ID NOs: 15, 16, and 17, respectively, including variants thereof with 1, 2, 3, 4, or 5 alterations in the CDR(s) and which specifically bind to the human HRS polypeptide;

the V_{H}CDR1, V_{H}CDR2, and V_{H}CDR3 sequences comprise SEQ ID NOs: 18, 19, and 20, respectively, and the V_{L}CDR1, V_{L}CDR2, and V_{L}CDR3 sequences comprise SEQ ID NOs: 21, 22, and 23, respectively, including variants thereof with 1, 2, 3, 4, or 5 alterations in the CDR(s) and which specifically bind to the human HRS polypeptide;

the V_{H}CDR1, V_{H}CDR2, and V_{H}CDR3 sequences comprise SEQ ID NOs: 24, 25, and 26, respectively, and the V_{L}CDR1, V_{L}CDR2, and V_{L}CDR3 sequences comprise SEQ ID NOs: 27, 28, and 29, respectively, including variants thereof with 1, 2, 3, 4, or 5 alterations in the CDR(s) and which specifically bind to the human HRS polypeptide;

the V_{H}CDR1, V_{H}CDR2, and V_{H}CDR3 sequences comprise SEQ ID NOs: 36, 37, and 38, respectively, and the V_{L}CDR1, V_{L}CDR2, and V_{L}CDR3 sequences comprise SEQ ID NOs: 39, 40, and 41, respectively, including variants thereof;

the V_{H}CDR1, V_{H}CDR2, and V_{H}CDR3 sequences comprise SEQ ID NOs: 42, 43, and 44, respectively, and the V_{L}CDR1, V_{L}CDR2, and V_{L}CDR3 sequences comprise SEQ ID NOs: 45, 46, and 47, respectively, including variants thereof;

the V_{H}CDR1, V_{H}CDR2, and V_{H}CDR3 sequences comprise SEQ ID NOs: 48, 49, and 50, respectively, and the V_{L}CDR1, V_{L}CDR2, and V_{L}CDR3 sequences comprise SEQ ID NOs: 51, 52, and 53, respectively, including variants thereof;

the V_{H}CDR1, V_{H}CDR2, and V_{H}CDR3 sequences comprise SEQ ID NOs: 54, 55, and 56, respectively, and the V_{L}CDR1, V_{L}CDR2, and V_{L}CDR3 sequences comprise SEQ ID NOs: 57, 58, and 59, respectively, including variants thereof;

the V_{H}CDR1, V_{H}CDR2, and V_{H}CDR3 sequences comprise SEQ ID NOs: 60, 61, and 62, respectively, and the V_{L}CDR1, V_{L}CDR2, and V_{L}CDR3 sequences comprise SEQ ID NOs: 63, 64, and 65, respectively, including variants thereof;

the V_{H}CDR1, V_{H}CDR2, and V_{H}CDR3 sequences comprise SEQ ID NOs: 66, 67, and 68, respectively, and the V_{L}CDR1, V_{L}CDR2, and V_{L}CDR3 sequences comprise SEQ ID NOs: 69, 70, and 71, respectively, including variants thereof;

the V_{H}CDR1, V_{H}CDR2, and V_{H}CDR3 sequences comprise SEQ ID NOs: 72, 73, and 74, respectively, and the V_{L}CDR1, V_{L}CDR2, and V_{L}CDR3 sequences comprise SEQ ID NOs: 75, 76, and 77, respectively, including variants thereof;

the V_{H}CDR1, V_{H}CDR2, and V_{H}CDR3 sequences comprise SEQ ID NOs: 78, 79, and 80, respectively, and the V_{L}CDR1, V_{L}CDR2, and V_{L}CDR3 sequences comprise SEQ ID NOs: 81, 82, and 83, respectively, including variants thereof;

the V_{H}CDR1, V_{H}CDR2, and V_{H}CDR3 sequences comprise SEQ ID NOs: 84, 85, and 86, respectively, and the V_{L}CDR1, V_{L}CDR2, and V_{L}CDR3 sequences comprise SEQ ID NOs: 87, 88, and 89, respectively, including variants thereof;

the V_{H}CDR1, V_{H}CDR2, and V_{H}CDR3 sequences comprise SEQ ID NOs: 90, 91, and 92, respectively, and the V_{L}CDR1, V_{L}CDR2, and V_{L}CDR3 sequences comprise SEQ ID NOs: 93, 94, and 95, respectively, including variants thereof;

the V_{H}CDR1, V_{H}CDR2, and V_{H}CDR3 sequences comprise SEQ ID NOs: 96, 97, and 98, respectively, and the V_{L}CDR1, V_{L}CDR2, and V_{L}CDR3 sequences comprise SEQ ID NOs: 99, 100, and 101, respectively, including variants thereof;

the V_{H}CDR1, V_{H}CDR2, and V_{H}CDR3 sequences comprise SEQ ID NOs: 102, 103, and 104, respectively, and the V_{L}CDR1, V_{L}CDR2, and V_{L}CDR3 sequences comprise SEQ ID NOs: 105, 106, and 107, respectively, including variants thereof;

the V_{H}CDR1, V_{H}CDR2, and V_{H}CDR3 sequences comprise SEQ ID NOs: 108, 109, and 110, respectively, and the V_{L}CDR1, V_{L}CDR2, and V_{L}CDR3 sequences comprise SEQ ID NOs: 111, 112, and 113, respectively, including variants thereof;

the V_{H}CDR1, V_{H}CDR2, and V_{H}CDR3 sequences comprise SEQ ID NOs: 114, 115, and 116, respectively, and the V_{L}CDR1, V_{L}CDR2, and V_{L}CDR3 sequences comprise SEQ ID NOs: 117, 118, and 119, respectively, including variants thereof;

the V_{H}CDR1, V_{H}CDR2, and V_{H}CDR3 sequences comprise SEQ ID NOs: 120, 121, and 122, respectively, and the V_{L}CDR1, V_{L}CDR2, and V_{L}CDR3 sequences comprise SEQ ID NOs: 123, 124, and 125, respectively, including variants thereof;

the V_{H}CDR1, V_{H}CDR2, and V_{H}CDR3 sequences comprise SEQ ID NOs: 126, 127, and 128, respectively, and the V_{L}CDR1, V_{L}CDR2, and V_{L}CDR3 sequences comprise SEQ ID NOs: 129, 130, and 131, respectively, including variants thereof;

the V_{H}CDR1, V_{H}CDR2, and V_{H}CDR3 sequences comprise SEQ ID NOs: 132, 133, and 134, respectively, and the V_{L}CDR1, V_{L}CDR2, and V_{L}CDR3 sequences comprise SEQ ID NOs: 135, 136, and 137, respectively, including variants thereof;

the V_{H}CDR1, V_{H}CDR2, and V_{H}CDR3 sequences comprise SEQ ID NOs: 138, 139, and 140, respectively, and the V_{L}CDR1, V_{L}CDR2, and V_{L}CDR3 sequences comprise SEQ ID NOs: 141, 142, and 143, respectively, including variants thereof;

the V_{H}CDR1, V_{H}CDR2, and V_{H}CDR3 sequences comprise SEQ ID NOs: 144, 145, and 146, respectively, and the V_{L}CDR1, V_{L}CDR2, and V_{L}CDR3 sequences comprise SEQ ID NOs: 147, 148, and 149, respectively, including variants thereof;

the V_{H}CDR1, V_{H}CDR2, and V_{H}CDR3 sequences comprise SEQ ID NOs: 150, 151, and 152, respectively, and the V_{L}CDR1, V_{L}CDR2, and V_{L}CDR3 sequences comprise SEQ ID NOs: 153, 154, and 155, respectively, including variants thereof;

the V_{H}CDR1, V_{H}CDR2, and V_{H}CDR3 sequences comprise SEQ ID NOs: 156, 157, and 158, respectively, and the V_{L}CDR1, V_{L}CDR2, and V_{L}CDR3 sequences comprise SEQ ID NOs: 159, 160, and 161, respectively, including variants thereof;

the V_{H}CDR1, V_{H}CDR2, and V_{H}CDR3 sequences comprise SEQ ID NOs: 162, 163, and 164, respectively, and the V_{L}CDR1, V_{L}CDR2, and V_{L}CDR3 sequences comprise SEQ ID NOs: 165, 166, and 167, respectively, including variants thereof;

the V_{H}CDR1, V_{H}CDR2, and V_{H}CDR3 sequences comprise SEQ ID NOs: 168, 169, and 170, respectively, and the V_{L}CDR1, V_{L}CDR2, and V_{L}CDR3 sequences comprise SEQ ID NOs: 171, 172, and 173, respectively, including variants thereof;

the V_{H}CDR1, V_{H}CDR2, and V_{H}CDR3 sequences comprise SEQ ID NOs: 174, 175, and 176, respectively, and the V_{L}CDR1, V_{L}CDR2, and V_{L}CDR3 sequences comprise SEQ ID NOs: 177, 178, and 179, respectively, including variants thereof;

the V_{H}CDR1, V_{H}CDR2, and V_{H}CDR3 sequences comprise SEQ ID NOs: 180, 181, and 182, respectively, and the V_{L}CDR1, V_{L}CDR2, and V_{L}CDR3 sequences comprise SEQ ID NOs: 183, 184, and 185, respectively, including variants thereof;

the V_{H}CDR1, V_{H}CDR2, and V_{H}CDR3 sequences comprise SEQ ID NOs: 186, 187, and 188, respectively, and the V_{L}CDR1, V_{L}CDR2, and V_{L}CDR3 sequences comprise SEQ ID NOs: 189, 190, and 191, respectively, including variants thereof;

the V_{H}CDR1, V_{H}CDR2, and V_{H}CDR3 sequences comprise SEQ ID NOs: 192, 193, and 194, respectively, and the V_{L}CDR1, V_{L}CDR2, and V_{L}CDR3 sequences comprise SEQ ID NOs: 195, 196, and 197, respectively, including variants thereof;

the V_{H}CDR1, V_{H}CDR2, and V_{H}CDR3 sequences comprise SEQ ID NOs: 198, 199, and 200, respectively, and the V_{L}CDR1, V_{L}CDR2, and V_{L}CDR3 sequences comprise SEQ ID NOs: 201, 202, and 203, respectively, including variants thereof;

the V_{H}CDR1, V_{H}CDR2, and V_{H}CDR3 sequences comprise SEQ ID NOs: 204, 205, and 206, respectively, and the V_{L}CDR1, V_{L}CDR2, and V_{L}CDR3 sequences comprise SEQ ID NOs: 207, 208, and 209, respectively, including variants thereof;

the V_{H}CDR1, V_{H}CDR2, and V_{H}CDR3 sequences comprise SEQ ID NOs: 210, 211, and 212, respectively, and the V_{L}CDR1, V_{L}CDR2, and V_{L}CDR3 sequences comprise SEQ ID NOs: 213, 214, and 215, respectively, including variants thereof;

the V_{H}CDR1, V_{H}CDR2, and V_{H}CDR3 sequences comprise SEQ ID NOs: 216, 217, and 218, respectively, and the V_{L}CDR1, V_{L}CDR2, and V_{L}CDR3 sequences comprise SEQ ID NOs: 219, 220, and 221, respectively, including variants thereof;

the V_{H}CDR1, V_{H}CDR2, and V_{H}CDR3 sequences comprise SEQ ID NOs: 222, 223, and 224, respectively, and the V_{L}CDR1, V_{L}CDR2, and V_{L}CDR3 sequences comprise SEQ ID NOs: 225, 226, and 227, respectively, including variants thereof;

the V_{H}CDR1, V_{H}CDR2, and V_{H}CDR3 sequences comprise SEQ ID NOs: 228, 229, and 230, respectively, and the V_{L}CDR1, V_{L}CDR2, and V_{L}CDR3 sequences comprise SEQ ID NOs: 231, 232, and 233, respectively, including variants thereof;

the V_{H}CDR1, V_{H}CDR2, and V_{H}CDR3 sequences comprise SEQ ID NOs: 234, 235, and 236, respectively, and the V_{L}CDR1, V_{L}CDR2, and V_{L}CDR3 sequences comprise SEQ ID NOs: 237, 238, and 239, respectively, including variants thereof;

the V_{H}CDR1, V_{H}CDR2, and V_{H}CDR3 sequences comprise SEQ ID NOs: 240, 241, and 242, respectively, and the V_{L}CDR1, V_{L}CDR2, and V_{L}CDR3 sequences comprise SEQ ID NOs: 243, 244, and 245, respectively, including variants thereof;

the V_{H}CDR1, V_{H}CDR2, and V_{H}CDR3 sequences comprise SEQ ID NOs: 246, 247, and 248, respectively, and the V_{L}CDR1, V_{L}CDR2, and V_{L}CDR3 sequences comprise SEQ ID NOs: 249, 250, and 251, respectively, including variants thereof;

the V_{H}CDR1, V_{H}CDR2, and V_{H}CDR3 sequences comprise SEQ ID NOs: 252, 253, and 254, respectively, and the V_{L}CDR1, V_{L}CDR2, and V_{L}CDR3 sequences comprise SEQ ID NOs: 255, 256, and 257, respectively, including variants thereof;

the V_{H}CDR1, V_{H}CDR2, and V_{H}CDR3 sequences comprise SEQ ID NOs: 258, 259, and 260, respectively, and the V_{L}CDR1, V_{L}CDR2, and V_{L}CDR3 sequences comprise SEQ ID NOs: 261, 262, and 263, respectively, including variants thereof;

the V_{H}CDR1, V_{H}CDR2, and V_{H}CDR3 sequences comprise SEQ ID NOs: 264, 265, and 266, respectively, and the V_{L}CDR1, V_{L}CDR2, and V_{L}CDR3 sequences comprise SEQ ID NOs: 267, 268, and 269, respectively, including variants thereof;

the V_{H}CDR1, V_{H}CDR2, and V_{H}CDR3 sequences comprise SEQ ID NOs: 270, 271, and 272, respectively, and the V_{L}CDR1, V_{L}CDR2, and V_{L}CDR3 sequences comprise SEQ ID NOs: 273, 274, and 275, respectively, including variants thereof;

the V_{H}CDR1, V_{H}CDR2, and V_{H}CDR3 sequences comprise SEQ ID NOs: 276, 277, and 278, respectively, and the V_{L}CDR1, V_{L}CDR2, and V_{L}CDR3 sequences comprise SEQ ID NOs: 279, 280, and 281, respectively, including variants thereof;

the V_{H}CDR1, V_{H}CDR2, and V_{H}CDR3 sequences comprise SEQ ID NOs: 282, 283, and 284, respectively, and the V_{L}CDR1, V_{L}CDR2, and V_{L}CDR3 sequences comprise SEQ ID NOs: 285, 286, and 287, respectively, including variants thereof;

the V_{H}CDR1, V_{H}CDR2, and V_{H}CDR3 sequences comprise SEQ ID NOs: 288, 289, and 290, respectively, and the V_{L}CDR1, V_{L}CDR2, and V_{L}CDR3 sequences comprise SEQ ID NOs: 291, 292, and 293, respectively, including variants thereof;

the V_{H}CDR1, V_{H}CDR2, and V_{H}CDR3 sequences comprise SEQ ID NOs: 294, 295, and 296, respectively, and the V_{L}CDR1, V_{L}CDR2, and V_{L}CDR3 sequences comprise SEQ ID NOs: 297, 298, and 299, respectively, including variants thereof; and/or

the V_{H}CDR1, V_{H}CDR2, and V_{H}CDR3 sequences comprise SEQ ID NOs: 300, 301, and 302, respectively, and the V_{L}CDR1, V_{L}CDR2, and V_{L}CDR3 sequences comprise SEQ ID NOs: 303, 304, and 305, respectively, including variants thereof;

including affinity matured variants of the foregoing which specifically bind to the human HRS polypeptide.

In some embodiments, the V_{H} sequence is at least 80, 85, 90, 95, 97, 98, 99, or 100% identical to a sequence selected from Table A2, optionally wherein the V_{H} sequence has 1, 2, 3, 4, or 5 alterations in the framework regions. In some embodiments, the V_{L} sequence is at least 80, 85, 90, 95, 97, 98, 99, or 100% identical to a sequence selected from Table A2, optionally wherein the V_{L} sequence has 1, 2, 3, 4, or 5 alterations in the framework regions.

In some embodiments:

the V_{H} sequence comprises a sequence at least 80, 85, 90, 95, 97, 98, 99, or 100% identical to SEQ ID NO:30, and the V_{L} sequence comprises a sequence at least 80, 85, 90, 95, 97, 98, 99, or 100% identical to SEQ ID NO:31;

the V_{H} sequence comprises a sequence at least 80, 85, 90, 95, 97, 98, 99, or 100% identical to SEQ ID NO:32, and the V_{L} sequence comprises a sequence at least 80, 85, 90, 95, 97, 98, 99, or 100% identical to SEQ ID NO:33;

the V_{H} sequence comprises a sequence at least 80, 85, 90, 95, 97, 98, 99, or 100% identical to SEQ ID NO:34, and the V_{L} sequence comprises a sequence at least 80, 85, 90, 95, 97, 98, 99, or 100% identical to SEQ ID NO:35;

the V_{H} sequence comprises a sequence at least 80, 85, 90, 95, 97, 98, 99, or 100% identical to SEQ ID NO:306, and the V_{L} sequence comprises a sequence at least 80, 85, 90, 95, 97, 98, 99, or 100% identical to SEQ ID NO:307;

the V_{H} sequence comprises a sequence at least 80, 85, 90, 95, 97, 98, 99, or 100% identical to SEQ ID NO:308, and the V_{L} sequence comprises a sequence at least 80, 85, 90, 95, 97, 98, 99, or 100% identical to SEQ ID NO:309;

the V_{H} sequence comprises a sequence at least 80, 85, 90, 95, 97, 98, 99, or 100% identical to SEQ ID NO:310, and the V_{L} sequence comprises a sequence at least 80, 85, 90, 95, 97, 98, 99, or 100% identical to SEQ ID NO:311;

the V_{H} sequence comprises a sequence at least 80, 85, 90, 95, 97, 98, 99, or 100% identical to SEQ ID NO:312, and the V_{L} sequence comprises a sequence at least 80, 85, 90, 95, 97, 98, 99, or 100% identical to SEQ ID NO:313;

the V_{H} sequence comprises a sequence at least 80, 85, 90, 95, 97, 98, 99, or 100% identical to SEQ ID NO:314, and the V_{L} sequence comprises a sequence at least 80, 85, 90, 95, 97, 98, 99, or 100% identical to SEQ ID NO:315;

the V_{H} sequence comprises a sequence at least 80, 85, 90, 95, 97, 98, 99, or 100% identical to SEQ ID NO:316, and the V_{L} sequence comprises a sequence at least 80, 85, 90, 95, 97, 98, 99, or 100% identical to SEQ ID NO:317;

the V_{H} sequence comprises a sequence at least 80, 85, 90, 95, 97, 98, 99, or 100% identical to SEQ ID NO:318, and the V_{L} sequence comprises a sequence at least 80, 85, 90, 95, 97, 98, 99, or 100% identical to SEQ ID NO:319;

the V_{H} sequence comprises a sequence at least 80, 85, 90, 95, 97, 98, 99, or 100% identical to SEQ ID NO:320, and the V_{L} sequence comprises a sequence at least 80, 85, 90, 95, 97, 98, 99, or 100% identical to SEQ ID NO:321;

the V_{H} sequence comprises a sequence at least 80, 85, 90, 95, 97, 98, 99, or 100% identical to SEQ ID NO:322, and the V_{L} sequence comprises a sequence at least 80, 85, 90, 95, 97, 98, 99, or 100% identical to SEQ ID NO:323;

the V_{H} sequence comprises a sequence at least 80, 85, 90, 95, 97, 98, 99, or 100% identical to SEQ ID NO:324, and the V_{L} sequence comprises a sequence at least 80, 85, 90, 95, 97, 98, 99, or 100% identical to SEQ ID NO:325;

the V_{H} sequence comprises a sequence at least 80, 85, 90, 95, 97, 98, 99, or 100% identical to SEQ ID NO:326, and the V_{L} sequence comprises a sequence at least 80, 85, 90, 95, 97, 98, 99, or 100% identical to SEQ ID NO:327;

the V_{H} sequence comprises a sequence at least 80, 85, 90, 95, 97, 98, 99, or 100% identical to SEQ ID NO:328, and the V_{L} sequence comprises a sequence at least 80, 85, 90, 95, 97, 98, 99, or 100% identical to SEQ ID NO:329;

the V_{H} sequence comprises a sequence at least 80, 85, 90, 95, 97, 98, 99, or 100% identical to SEQ ID NO:330, and the V_{L} sequence comprises a sequence at least 80, 85, 90, 95, 97, 98, 99, or 100% identical to SEQ ID NO:331;

the V_{H} sequence comprises a sequence at least 80, 85, 90, 95, 97, 98, 99, or 100% identical to SEQ ID NO:332, and the V_{L} sequence comprises a sequence at least 80, 85, 90, 95, 97, 98, 99, or 100% identical to SEQ ID NO:333;

the V_{H} sequence comprises a sequence at least 80, 85, 90, 95, 97, 98, 99, or 100% identical to SEQ ID NO:334, and the V_{L} sequence comprises a sequence at least 80, 85, 90, 95, 97, 98, 99, or 100% identical to SEQ ID NO:335;

the V_{H} sequence comprises a sequence at least 80, 85, 90, 95, 97, 98, 99, or 100% identical to SEQ ID NO:336, and the V_{L} sequence comprises a sequence at least 80, 85, 90, 95, 97, 98, 99, or 100% identical to SEQ ID NO:337;

the V_{H} sequence comprises a sequence at least 80, 85, 90, 95, 97, 98, 99, or 100% identical to SEQ ID NO:338, and the V_{L} sequence comprises a sequence at least 80, 85, 90, 95, 97, 98, 99, or 100% identical to SEQ ID NO:339;

the V_{H} sequence comprises a sequence at least 80, 85, 90, 95, 97, 98, 99, or 100% identical to SEQ ID NO:340, and the V_{L} sequence comprises a sequence at least 80, 85, 90, 95, 97, 98, 99, or 100% identical to SEQ ID NO:341;

the V_{H} sequence comprises a sequence at least 80, 85, 90, 95, 97, 98, 99, or 100% identical to SEQ ID NO:342, and the V_{L} sequence comprises a sequence at least 80, 85, 90, 95, 97, 98, 99, or 100% identical to SEQ ID NO:343;

the V_{H} sequence comprises a sequence at least 80, 85, 90, 95, 97, 98, 99, or 100% identical to SEQ ID NO:344, and the V_{L} sequence comprises a sequence at least 80, 85, 90, 95, 97, 98, 99, or 100% identical to SEQ ID NO:345;

the V_{H} sequence comprises a sequence at least 80, 85, 90, 95, 97, 98, 99, or 100% identical to SEQ ID NO:346, and the V_{L} sequence comprises a sequence at least 80, 85, 90, 95, 97, 98, 99, or 100% identical to SEQ ID NO:347;

the V_{H} sequence comprises a sequence at least 80, 85, 90, 95, 97, 98, 99, or 100% identical to SEQ ID NO:348, and the V_{L} sequence comprises a sequence at least 80, 85, 90, 95, 97, 98, 99, or 100% identical to SEQ ID NO:349;

the V_{H} sequence comprises a sequence at least 80, 85, 90, 95, 97, 98, 99, or 100% identical to SEQ ID NO:350, and the V_{L} sequence comprises a sequence at least 80, 85, 90, 95, 97, 98, 99, or 100% identical to SEQ ID NO:351;

the V_{H} sequence comprises a sequence at least 80, 85, 90, 95, 97, 98, 99, or 100% identical to SEQ ID NO:352, and the V_{L} sequence comprises a sequence at least 80, 85, 90, 95, 97, 98, 99, or 100% identical to SEQ ID NO:353;

the V_{H} sequence comprises a sequence at least 80, 85, 90, 95, 97, 98, 99, or 100% identical to SEQ ID NO:354, and the V_{L} sequence comprises a sequence at least 80, 85, 90, 95, 97, 98, 99, or 100% identical to SEQ ID NO:355;

the V_{H} sequence comprises a sequence at least 80, 85, 90, 95, 97, 98, 99, or 100% identical to SEQ ID NO:356, and the V_{L} sequence comprises a sequence at least 80, 85, 90, 95, 97, 98, 99, or 100% identical to SEQ ID NO:357;

the V_{H} sequence comprises a sequence at least 80, 85, 90, 95, 97, 98, 99, or 100% identical to SEQ ID NO:358, and the V_{L} sequence comprises a sequence at least 80, 85, 90, 95, 97, 98, 99, or 100% identical to SEQ ID NO:359;

the V_{H} sequence comprises a sequence at least 80, 85, 90, 95, 97, 98, 99, or 100% identical to SEQ ID NO:360, and the V_{L} sequence comprises a sequence at least 80, 85, 90, 95, 97, 98, 99, or 100% identical to SEQ ID NO:361;

the V_{H} sequence comprises a sequence at least 80, 85, 90, 95, 97, 98, 99, or 100% identical to SEQ ID NO:362, and the V_{L} sequence comprises a sequence at least 80, 85, 90, 95, 97, 98, 99, or 100% identical to SEQ ID NO:363;

the V_{H} sequence comprises a sequence at least 80, 85, 90, 95, 97, 98, 99, or 100% identical to SEQ ID NO:364, and the V_{L} sequence comprises a sequence at least 80, 85, 90, 95, 97, 98, 99, or 100% identical to SEQ ID NO:365;

the V_{H} sequence comprises a sequence at least 80, 85, 90, 95, 97, 98, 99, or 100% identical to SEQ ID NO:366, and the V_{L} sequence comprises a sequence at least 80, 85, 90, 95, 97, 98, 99, or 100% identical to SEQ ID NO:367;

the V_{H} sequence comprises a sequence at least 80, 85, 90, 95, 97, 98, 99, or 100% identical to SEQ ID NO:368, and the V_{L} sequence comprises a sequence at least 80, 85, 90, 95, 97, 98, 99, or 100% identical to SEQ ID NO:369;

the V_{H} sequence comprises a sequence at least 80, 85, 90, 95, 97, 98, 99, or 100% identical to SEQ ID NO:370, and the V_{L} sequence comprises a sequence at least 80, 85, 90, 95, 97, 98, 99, or 100% identical to SEQ ID NO:371;

the V_{H} sequence comprises a sequence at least 80, 85, 90, 95, 97, 98, 99, or 100% identical to SEQ ID NO:372, and the V_{L} sequence comprises a sequence at least 80, 85, 90, 95, 97, 98, 99, or 100% identical to SEQ ID NO:373;

the V_{H} sequence comprises a sequence at least 80, 85, 90, 95, 97, 98, 99, or 100% identical to SEQ ID NO:374, and the V_{L} sequence comprises a sequence at least 80, 85, 90, 95, 97, 98, 99, or 100% identical to SEQ ID NO:375;

the V_{H} sequence comprises a sequence at least 80, 85, 90, 95, 97, 98, 99, or 100% identical to SEQ ID NO:376, and the V_{L} sequence comprises a sequence at least 80, 85, 90, 95, 97, 98, 99, or 100% identical to SEQ ID NO:377;

the V_{H} sequence comprises a sequence at least 80, 85, 90, 95, 97, 98, 99, or 100% identical to SEQ ID NO:378, and the V_{L} sequence comprises a sequence at least 80, 85, 90, 95, 97, 98, 99, or 100% identical to SEQ ID NO:379;

the V_{H} sequence comprises a sequence at least 80, 85, 90, 95, 97, 98, 99, or 100% identical to SEQ ID NO:380, and the V_{L} sequence comprises a sequence at least 80, 85, 90, 95, 97, 98, 99, or 100% identical to SEQ ID NO:381;

the V_{H} sequence comprises a sequence at least 80, 85, 90, 95, 97, 98, 99, or 100% identical to SEQ ID NO:382, and the V_{L} sequence comprises a sequence at least 80, 85, 90, 95, 97, 98, 99, or 100% identical to SEQ ID NO:383;

the V_{H} sequence comprises a sequence at least 80, 85, 90, 95, 97, 98, 99, or 100% identical to SEQ ID NO:384, and the V_{L} sequence comprises a sequence at least 80, 85, 90, 95, 97, 98, 99, or 100% identical to SEQ ID NO:385;

the V_{H} sequence comprises a sequence at least 80, 85, 90, 95, 97, 98, 99, or 100% identical to SEQ ID NO:386, and the V_{L} sequence comprises a sequence at least 80, 85, 90, 95, 97, 98, 99, or 100% identical to SEQ ID NO:387;

the V_{H} sequence comprises a sequence at least 80, 85, 90, 95, 97, 98, 99, or 100% identical to SEQ ID NO:388, and the V_{L} sequence comprises a sequence at least 80, 85, 90, 95, 97, 98, 99, or 100% identical to SEQ ID NO:389;

the V_{H} sequence comprises a sequence at least 80, 85, 90, 95, 97, 98, 99, or 100% identical to SEQ ID NO:390, and the V_{L} sequence comprises a sequence at least 80, 85, 90, 95, 97, 98, 99, or 100% identical to SEQ ID NO:391;

the V_{H} sequence comprises a sequence at least 80, 85, 90, 95, 97, 98, 99, or 100% identical to SEQ ID NO:392, and the V_{L} sequence comprises a sequence at least 80, 85, 90, 95, 97, 98, 99, or 100% identical to SEQ ID NO:393; and/or

the V_{H} sequence comprises a sequence at least 80, 85, 90, 95, 97, 98, 99, or 100% identical to SEQ ID NO:394, and the V_{L} sequence comprises a sequence at least 80, 85, 90, 95, 97, 98, 99, or 100% identical to SEQ ID NO:395.

In some embodiments, the at least one antibody or antigen-binding fragment thereof is a monoclonal antibody. In some embodiments, the at least one antibody or antigen-binding fragment thereof is a humanized antibody. In some embodiments, the at least one antibody or antigen-binding fragment thereof is an Fv fragment, a single chain Fv (scFv) polypeptide, an adnectin, an anticalin, an aptamer, an avimer, a camelid antibody, a designed ankyrin repeat protein (DARPin), a minibody, a nanobody, or a unibody.

Some compositions comprise at least two anti-HRS antibodies, comprising a first antibody or antigen-binding fragment thereof that specifically binds to at least one first epitope of a human HRS polypeptide, and a second antibody or antigen-binding fragment thereof that specifically binds to at least one second epitope of a human HRS polypeptide, optionally wherein the at least one first epitope differs from the at least one second epitope.

In some embodiments, the first and the second antibodies or antigen-binding fragments thereof specifically and non-competitively bind to the same domain of the HRS polypeptide, optionally wherein the first and the second antibodies or antigen-binding fragments thereof specifically bind to the N-terminal domain, the aminoacylation domain, or the anticodon binding domain. In some embodiments, the first and the second antibodies or antigen-binding fragments thereof specifically and non-competitively bind to different domains of the HRS polypeptide. In some embodiments, the first antibody or antigen-binding fragment thereof specifically binds to the N-terminal domain, and the second antibody or antigen-binding fragment thereof specifically binds to the aminoacylation domain. In some embodiments, the first antibody or antigen-binding fragment thereof specifically binds to the N-terminal domain, and the second antibody or antigen-binding fragment thereof specifically binds to the anticodon binding domain. In some embodiments, the first antibody or antigen-binding fragment thereof specifically binds to the aminoacylation domain, and the second antibody or antigen-binding fragment thereof specifically binds to the anticodon binding. In some embodiments, the first and the second antibodies or antigen-binding fragments thereof are both blocking antibodies, or wherein the first and the second antibodies or antigen-binding fragments thereof are both partial-blocking antibodies, or wherein the first and the second antibodies or antigen-binding fragments thereof are both non-blocking antibodies. In some embodiments, the first antibody or antigen-binding fragment thereof is a blocking antibody and the second antibody or antigen-binding fragment thereof is a partial-blocking antibody, or wherein the first antibody or antigen-binding fragment thereof is a blocking antibody and the second antibody or antigen-binding fragment thereof is a non-blocking antibody. In some embodiments, the first and the second antibodies or antigen-binding fragments thereof both comprise an IgG Fc domain with high effector function in humans, optionally an IgG1 or IgG3 Fc domain, or wherein the first and the second antibodies or antigen-binding fragments thereof both comprise an IgG Fc domain with low effector function in humans, optionally an IgG2 or IgG4 Fc domain. In some embodiments, the first antibody or antigen-binding fragment thereof comprises an IgG Fc domain with high effector function in humans, optionally an IgG1 or IgG3 Fc domain, and wherein the second antibody or antigen-binding fragment thereof comprises an IgG Fc domain with low effector function in humans, optionally an IgG2 or IgG4 Fc domain.

In some embodiments, the at least one antibody or antigen-binding fragment thereof comprises a polyclonal mixture of naturally-occurring antibodies obtained from one or more donor subjects, optionally wherein the polyclonal mixture has an average affinity (Kd) for the HRS polypeptide of about, at least about, or less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In some embodiments, the polyclonal mixture comprises or consists of human anti-Jo-1 antibodies, which are optionally obtained from one or more human donor subjects having an anti-Jo-1 antibody serum level of about or at least about 0.1 μg/mL, 0.2 μg/mL, 0.5 μg/mL, 1 μg/mL, 2 μg/mL, 5 μg/mL, 10 μg/mL, 20 g/mL, 50 μg/mL, or 100 μg/mL. In some embodiments, the polyclonal mixture is a serum or plasma preparation obtained from the one or more donor subjects, wherein the preparation is substantially-free of other serum immunoglobulins and optionally comprises about or at least about 1 μg/mL, 2 μg/mL, 5 g/mL, 10 μg/mL, 20 μg/mL, 50 μg/mL, 100 μg/mL, 1 mg/mL, 2 mg/mL, 5 mg/mL, 10 mg/mL, or 100 mg/mL of the naturally-occurring anti-Jo-1 antibodies. In some embodiments, the polyclonal mixture is a serum or plasma preparation obtained from the one or more donor subjects, wherein the preparation comprises other serum immunoglobulins and optionally comprises about or at least about 1 μg/mL, 2 g/mL, 5 μg/mL, 10 μg/mL, 20 μg/mL, 50 μg/mL, 100 μg/mL, 1 mg/mL, 2 mg/mL, 5 mg/mL, 10 mg/mL, or 100 mg/mL of the naturally-occurring anti-Jo-1 antibodies. In some embodiments, the polyclonal mixture is an Intravenous Immunoglobulin (IVIG) preparation obtained from the one or more donor subjects, which optionally comprises about or at least about 1 μg/mL, 2 μg/mL, 5 μg/mL, 10 g/mL, 20 μg/mL, 50 μg/mL, 100 μg/mL, 1 mg/mL, 2 mg/mL, 5 mg/mL, 10 mg/mL, or 100 mg/mL of the naturally-occurring anti-Jo-1 antibodies, and which is optionally supplemented with one or more recombinant anti-HRS antibodies to create an IVIG preparation with a total anti-HRS antibody level of about or at least about 100 μg/mL, 1 mg/mL, 2 mg/mL, 5 mg/mL, 10 mg/mL, or 100 mg/mL.

In some embodiments, the composition has a purity of at least about 80%, 85%, 90%, 95%, 98%, or 99% on a protein basis with respect to the at least one antibody or antigen-binding fragment, and is substantially aggregate-free. In some embodiments, the therapeutic composition is substantially endotoxin-free. In some embodiments, the therapeutic composition is a sterile, injectable solution, optionally suitable for intravenous, intramuscular, subcutaneous, or intraperitoneal administration.

Certain therapeutic compositions further comprise at least one cancer immunotherapy agent In some embodiments, the cancer immunotherapy agent is selected from one or more of an immune checkpoint modulatory agent, a cancer vaccine, an oncolytic virus, a cytokine, and a cell-based immunotherapies. In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule.

In some embodiments, the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736).

In some embodiments, the antagonist is a PD-1 antagonist optionally selected from one or more of an antibody or antigen-binding fragment or small molecule that specifically binds thereto, nivolumab, pembrolizumab, MK-3475, AMP-224, AMP-514, PDR001, and pidilizumab.

In some embodiments, the antagonist is a CTLA-4 antagonist optionally selected from one or more of an antibody or antigen-binding fragment or small molecule that specifically binds thereto, ipilimumab, and tremelimumab.

In some embodiments, the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat.

In some embodiments, the antagonist is a TDO antagonist optionally selected from one or more of an antibody or antigen-binding fragment or small molecule that specifically binds thereto, 680C91.

In some embodiments, the antagonist is a TIM-3 antagonist optionally selected from one or more of an antibody or antigen-binding fragment or small molecule that specifically binds thereto.

In some embodiments, the antagonist is a LAG-3 antagonist optionally selected from one or more of an antibody or antigen-binding fragment or small molecule that specifically binds thereto, and BMS-986016.

In some embodiments, the antagonist is a VISTA antagonist optionally selected from one or more of an antibody or antigen-binding fragment or small molecule that specifically binds thereto.

In some embodiments, the antagonist is a BTLA, CD160, and/or HVEM antagonist optionally selected from one or more of an antibody or antigen-binding fragment or small molecule that specifically binds thereto.

In some embodiments, the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998.

In some embodiments, the agonist is a CD40 agonist optionally selected from one or more of an antibody or antigen-binding fragment or small molecule or ligand that specifically binds thereto, CP-870, 893, dacetuzumab, Chi Lob 7/4, ADC-1013, and rhCD40L.

In some embodiments, the agonist is a GITR agonist optionally selected from one or more of an antibody or antigen-binding fragment or small molecule or ligand that specifically binds thereto, INCAGN01876, DTA-1, and MEDI1873.

In some embodiments, the agonist is a CD137 agonist optionally selected from one or more of an antibody or antigen-binding fragment or small molecule or ligand that specifically binds thereto, utomilumab, and 4-1BB ligand.

In some embodiments, the agonist is a CD27 agonist optionally selected from one or more of an antibody or antigen-binding fragment or small molecule or ligand that specifically binds thereto, varlilumab, and CDX-1127 (1F5).

In some embodiments, the agonist is a CD28 agonist optionally selected from one or more of an antibody or antigen-binding fragment or small molecule or ligand that specifically binds thereto, and TAB08.

In some embodiments, the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-α, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

Also included are methods of treating a cancer in a subject in need thereof, comprising administering to the subject a therapeutic composition comprising at least one antibody or antigen-binding fragment thereof that specifically binds to a human histidyl-tRNA synthetase (HRS) polypeptide (an anti-HRS antibody), optionally as a therapeutic composition described herein. Some methods include reducing or preventing re-emergence of a cancer in a subject in need thereof, wherein administration of the therapeutic composition enables generation of an immune memory to the cancer. In some embodiments, the subject has or is at risk for developing diabetes. Some embodiments comprise administering to the subject at least one cancer immunotherapy agent, which is optionally as defined herein.

In some embodiments, the at least one anti-HRS antibody and the at least one cancer immunotherapy agent are administered separately, as separate compositions. In some embodiments, the at least one anti-HRS antibody and the at least one cancer immunotherapy agent are administered together as part of the same therapeutic composition, optionally as a therapeutic composition as described herein.

In some embodiments, the cancer immunotherapy agent is selected from one or more of an immune checkpoint modulatory agent, a cancer vaccine, an oncolytic virus, a cytokine, and a cell-based immunotherapies. In some embodiments, the immune checkpoint modulatory agent is a polypeptide, optionally an antibody or antigen-binding fragment thereof or a ligand, or a small molecule. In some embodiments, the immune checkpoint modulatory agent comprises

(a) an antagonist of a inhibitory immune checkpoint molecule; or

(b) an agonist of a stimulatory immune checkpoint molecule.

In some embodiments, the immune checkpoint modulatory agent specifically binds to the immune checkpoint molecule.

In some embodiments, the inhibitory immune checkpoint molecule is selected from one or more of Programmed Death-Ligand 1 (PD-L1), Programmed Death 1 (PD-1), Programmed Death-Ligand 2 (PD-L2), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, Herpes Virus Entry Mediator (HVEM), and T-cell immunoreceptor with Ig and ITIM domains (TIGIT).

In some embodiments, the antagonist is a PD-L1 and/or PD-L2 antagonist optionally selected from one or more of an antibody or antigen-binding fragment or small molecule that specifically binds thereto, atezolizumab (MPDL3280A), avelumab (MSB0010718C), and durvalumab (MEDI4736). In some embodiments, the cancer is selected from one or more of colorectal cancer, melanoma, breast cancer, non-small-cell lung carcinoma, bladder cancer, and renal cell carcinoma.

In some embodiments, the antagonist is a PD-1 antagonist optionally selected from one or more of an antibody or antigen-binding fragment or small molecule that specifically binds thereto, nivolumab, pembrolizumab, MK-3475, AMP-224, AMP-514PDR001, and pidilizumab.

In some embodiments, the PD-1 antagonist is nivolumab and the cancer is optionally selected from one or more of Hodgkin's lymphoma, melanoma, non-small cell lung cancer, hepatocellular carcinoma, renal cell carcinoma, and ovarian cancer.

In some embodiments, the PD-1 antagonist is pembrolizumab and the cancer is optionally selected from one or more of melanoma, non-small cell lung cancer, small cell lung cancer, head and neck cancer, and urothelial cancer.

In some embodiments, the antagonist is a CTLA-4 antagonist optionally selected from one or more of an antibody or antigen-binding fragment or small molecule that specifically binds thereto, ipilimumab, tremelimumab. In some embodiments, the cancer is selected from one or more of melanoma, prostate cancer, lung cancer, and bladder cancer.

In some embodiments, the antagonist is an IDO antagonist optionally selected from one or more of an antibody or antigen-binding fragment or small molecule that specifically binds thereto, indoximod (NLG-8189), 1-methyl-tryptophan (1MT), β-Carboline (norharmane; 9H-pyrido[3,4-b]indole), rosmarinic acid, and epacadostat, and wherein the cancer is optionally selected from one or more of metastatic breast cancer and brain cancer optionally glioblastoma multiforme, glioma, gliosarcoma or malignant brain tumor.

In some embodiments, the antagonist is a TDO antagonist optionally selected from one or more of an antibody or antigen-binding fragment or small molecule that specifically binds thereto, 680C91, and LM10.

In some embodiments, the antagonist is a TIM-3 antagonist optionally selected from one or more of an antibody or antigen-binding fragment or small molecule that specifically binds thereto.

In some embodiments, the antagonist is a LAG-3 antagonist optionally selected from one or more of an antibody or antigen-binding fragment or small molecule that specifically binds thereto, and BMS-986016.

In some embodiments, the antagonist is a VISTA antagonist optionally selected from one or more of an antibody or antigen-binding fragment or small molecule that specifically binds thereto.

In some embodiments, the antagonist is a BTLA, CD160, and/or HVEM antagonist optionally selected from one or more of an antibody or antigen-binding fragment or small molecule that specifically binds thereto.

In some embodiments, the antagonist is a TIGIT antagonist optionally selected from one or more of an antibody or antigen-binding fragment or small molecule that specifically binds thereto.

In some embodiments, the stimulatory immune checkpoint molecule is selected from one or more of OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agonist is an OX40 agonist optionally selected from one or more of an antibody or antigen-binding fragment or small molecule or ligand that specifically binds thereto, OX86, Fc-OX40L, and GSK3174998.

In some embodiments, the agonist is a CD40 agonist optionally selected from one or more of an antibody or antigen-binding fragment or small molecule or ligand that specifically binds thereto, CP-870,893, dacetuzumab, Chi Lob 7/4, ADC-1013, and rhCD40L, and wherein the cancer is optionally selected from one or more of melanoma, pancreatic carcinoma, mesothelioma, and hematological cancers optionally lymphoma such as Non-Hodgkin's lymphoma.

In some embodiments, the agonist is a GITR agonist optionally selected from one or more of an antibody or antigen-binding fragment or small molecule or ligand that specifically binds thereto, INCAGN01876, DTA-1, and MEDI1873.

In some embodiments, the agonist is a CD137 agonist optionally selected from one or more of an antibody or antigen-binding fragment or small molecule or ligand that specifically binds thereto, utomilumab, and 4-1BB ligand.

In some embodiments, the agonist is a CD27 agonist optionally selected from one or more of an antibody or antigen-binding fragment or small molecule or ligand that specifically binds thereto, varlilumab, and CDX-1127 (1F5).

In some embodiments, the agonist is a CD28 agonist optionally selected from one or more of an antibody or antigen-binding fragment or small molecule or ligand that specifically binds thereto, and TAB08.

In some embodiments, the agonist is an HVEM agonist optionally selected from one or more of an antibody or antigen-binding fragment or small molecule or ligand that specifically binds thereto.

In some embodiments, the cancer vaccine is selected from one or more of Oncophage, a human papillomavirus HPV vaccine optionally Gardasil or Cervarix, a hepatitis B vaccine optionally Engerix-B, Recombivax HB, or Twinrix, and sipuleucel-T (Provenge), or comprises a cancer antigen selected from one or more of human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF1B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin, optionally wherein the subject has or is at risk for having a cancer that comprises the corresponding cancer antigen.

In some embodiments, the oncolytic virus selected from one or more of talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401.

In some embodiments, the cytokine selected from one or more of interferon (IFN)-α, IL-2, IL-12, IL-7, IL-21, and Granulocyte-macrophage colony-stimulating factor (GM-CSF).

In some embodiments, the cell-based immunotherapy agent comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells.

In some embodiments, the cancer is a primary cancer. In some embodiments, the cancer is a metastatic cancer. In some embodiments, the cancer is selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioma, glioblastoma multiforme, meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, and stomach cancer.

In some embodiments, the metastatic cancer is selected from one or more of:

(a) a bladder cancer which has metastasized to the bone, liver, and/or lungs;

(b) a breast cancer which has metastasized to the bone, brain, liver, and/or lungs;

(c) a colorectal cancer which has metastasized to the liver, lungs, and/or peritoneum;

(d) a kidney cancer which has metastasized to the adrenal glands, bone, brain, liver, and/or lungs;

(e) a lung cancer which has metastasized to the adrenal glands, bone, brain, liver, and/or other lung sites;

(f) a melanoma which has metastasized to the bone, brain, liver, lung, and/or skin/muscle;

(g) a ovarian cancer which has metastasized to the liver, lung, and/or peritoneum;

(h) a pancreatic cancer which has metastasized to the liver, lung, and/or peritoneum;

(i) a prostate cancer which has metastasized to the adrenal glands, bone, liver, and/or lungs;

(j) a stomach cancer which has metastasized to the liver, lung, and/or peritoneum;

(l) a thyroid cancer which has metastasized to the bone, liver, and/or lungs; and

(m) a uterine cancer which has metastasized to the bone, liver, lung, peritoneum, and/or vagina.

In some embodiments, the subject has, and/or is selected for treatment based on having, increased circulating or serum levels of at least one HRS polypeptide (optionally selected from Table H1), either bound or free, relative to the levels of a healthy or matched control standard or population of subject(s), optionally about or at least about 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 3000, 4000, or 5000 pM of the at least one HRS polypeptide, or about or at least about 30-100, 40-100, 50-100, 30-2000, 40-2000, 50-2000, 60-2000, 70-2000, 80-2000, 90-2000, 100-2000, 200-2000, 300-2000, 400-2000, 500-2000, 600-2000, 700-2000, 800-2000, 900-2000, 1000-2000, 2000-3000, 3000-4000, or 4000-5000 pM of the at least one HRS polypeptide.

In some embodiments, the subject has, and/or is selected for treatment based on having, a cancer which has increased levels or expression of an HRS polypeptide (optionally selected from Table H1) and/or a coding mRNA thereof relative to a non-cancerous control cell or tissue, optionally relative to a non-cancerous cell or tissue of the same type as the cancer, optionally wherein the HRS polypeptide is a splice variant selected from SV9, SV11, and SV14.

In some embodiments, the subject has, and/or is selected for treatment based on having, increased circulating or serum levels of a soluble neuropilin 2 (NP2) polypeptide (optionally selected from Table N1), either bound or free, relative to the levels of a healthy or matched control standard or population of subject(s), optionally circulating or serum levels of about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NP2 polypeptide, or optionally circulating or serum levels about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NP2 polypeptide.

In some embodiments, the subject has, and/or is selected for treatment based on having, a cancer which has increased levels or expression of an NP2 polypeptide (optionally selected from Table N1) and/or a coding mRNA thereof relative to a non-cancerous control cell or tissue, optionally relative to a non-cancerous cell or tissue of the same type as the cancer.

In some embodiments, the subject has, and/or is selected for treatment based on having, increased circulating levels of HRS:NP2 complexes relative to a healthy or matched control standard or population of subject(s).

Some embodiments comprise administering the at least one anti-HRS antibody in an amount and at a frequency sufficient to reduce the average or maximum levels of at least one serum or circulating HRS polypeptide (optionally selected from Table H1) to about or less than about 500 pM, 400 pM, 300 pM, 200 pM, 100 pM, 50 pm, 40 pM, 30 pM, 20 pM, or 10 pM.

Some embodiments comprise administering the at least one anti-HRS antibody in an amount and at a frequency sufficient to achieve an average, sustained serum or circulating levels of a soluble NP2 polypeptide of about or less than about 500 pM, 400 pM, 300 pM, 200 pM, 100 pM, 50 pm, 40 pM, 30 pM, 20 pM, or 10 pM.

Some embodiments comprise administering the at least one anti-HRS antibody in an amount and at a frequency sufficient to achieve a reduction in the circulating levels of HRS:NP2 complexes, optionally a reduction of about or at least about 5, 10, 15, 20, 25, 30, 35, 40, 45, 50, 60, 70, 80, 90, 95, 99, or 100%.

In some embodiments, the at least one anti-HRS antibody enhances the immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control.

In some embodiments, the at least one anti-HRS antibody enhances an anti-tumor and/or immunostimulatory activity of the cancer immunotherapy agent by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

Some embodiments comprise administering the at least one anti-HRS antibody in an amount and at a frequency sufficient to achieve a steady state concentration, or average circulating concentration, of the at least one anti-HRS antibody of between about 1 nM and about 1 μM, between about 1 nM and about 100 nM, between about 1 nM and about 10 nM, or between about 1 nM and about 3 μM

In some embodiments, the subject is a non-human mammalian subject, comprising administering a veterinary therapeutic composition comprising at least one antibody or antigen-binding fragment thereof specifically binds to a non-human mammalian HRS polypeptide, optionally selected from Table H2, including a dog, cat, pig, horse, or monkey HRS polypeptide.

Also included are veterinary therapeutic compositions, comprising at least one antibody or antigen-binding fragment thereof that specifically binds to a non-human mammalian HRS polypeptide, optionally selected from Table H2, including a dog, cat, pig, horse, or monkey HRS polypeptide.

Also included are patient care kits, comprising:

(a) at least one antibody or antigen-binding fragment thereof that specifically binds to a human histidyl-tRNA synthetase (HRS) polypeptide (an anti-HRS antibody); and optionally

(b) at least one cancer immunotherapy agent.

In some embodiments, (a) and (b) are in separate therapeutic compositions.

In some embodiments, (a) and (b) are in the same therapeutic composition.

Also included are bioassay systems, comprising a substantially pure anti-HRS antibody or antigen-binding fragment thereof, optionally as defined herein, a HRS polypeptide that binds to the anti-HRS antibody, and a host cell line that expresses neuropilin-2 on the cell surface.

In some embodiments, the HRS polypeptide is labelled with a detectable label. In some embodiments, the anti-HRS antibody is labelled with a detectable label. In some embodiments, the neuropilin 2 receptor is functionally coupled to a readout or indicator, such as a fluorescent or luminescent indicator of biological activity of the HRS polypeptide or neuropilin 2 receptor. In some embodiments, the HRS polypeptide is selected from Table H1 or Table H2. In some embodiments, the HRS polypeptide is comprises a WHEP domain. In some embodiments, the HRS polypeptide is comprises an aminoacylation domain. In some embodiments, the HRS polypeptide is comprises an anticodon binding domain. In some embodiments, the HRS polypeptide is comprises a HRS splice variant. In some embodiments, the HRS splice variant is selected from SV9, SV11 and SV14.

Also included are detection systems, comprising a cell that expresses a neuropilin 2 receptor or an extracellular portion thereof, and also expresses a recombinant HRS polypeptide, and a human or humanized anti-HARS antibody or antigen-binding fragment thereof that modulates the interaction of the HRS polypeptide and the neuropilin 2 receptor or the extracellular portion thereof. In some embodiments, the anti-HRS antibody is labelled with a detectable label. In some embodiments, the HRS polypeptide is selected from Table H1 or Table H2. In some embodiments, the HRS polypeptide is comprises a WHEP domain. In some embodiments, the HRS polypeptide is comprises an aminoacylation domain. In some embodiments, the HRS polypeptide is comprises an anticodon binding domain. In some embodiments, the HRS polypeptide is comprises a HRS splice variant. In some embodiments, the HRS splice variant is selected from SV9, SV11 and SV14. In some embodiments, the neuropilin 2 receptor is functionally coupled to a readout or indicator, such as a fluorescent or luminescent indicator of biological activity of the HRS polypeptide or neuropilin 2 receptor.

Also included are diagnostic systems, comprising a cell that comprises a neuropilin 2 receptor or an extracellular portion thereof, and a HRS polypeptide that specifically binds to the neuropilin 2 receptor, wherein the cell comprises an indicator molecule that allows detection of a change in the levels or activity of the cell-surface receptor or extracellular portion thereof, in response to interaction with the HRS polypeptide.

Also included are cellular compositions, comprising an engineered population of cells in which at least one cell comprises a polynucleotide encoding a human or humanized anti-HRS antibody, that comprises polynucleotide sequences encoding at least one amino acid sequence as set forth in Table A1 or Table A2, wherein the cells are capable of growing in a serum-free medium.

Also included are cellular growth devices, comprising a human or humanized anti-HRS antibody that comprises at least one amino acid sequence as set forth in any of Table A1 or Table A2, an engineered population of cells in which at least one cell comprises a polynucleotide encoding said anti-HRS antibody, at least about 10 liters of a serum-free growth medium, and a sterile container.

### BRIEF DESCRIPTION OF THE DRAWINGS

FIG. 1 provides an illustration of certain structural/functional domains of human histidyl-tRNA synthetase.

FIGS. 2A-2B show that anti-HRS antibodies inhibit B16-F10 melanoma growth in an in vivo syngeneic mouse model more effectively than the combination of anti-PDL1 and anti-CTLA4 antibodies.

FIG. 2A shows the impact of IgG control antibody (circles); the combination of anti-PD-L1 and anti-CTLA4 antibodies (Squares) and the combination of an N-terminally directed (clone 13E9) and C-terminally directed antibody (clone 13C8) to HRS (Triangles) on the average B16-F10 melanoma tumor volume over the study duration. FIG. 2B shows the same groups plotted at day 15 as a scatter plot; horizontal lines indicate group mean. Stars indicate significance vs. control, via 1-way ANOVA, Dunnett's post-hoc test. * p≤0.05, *** p<0.001.

FIG. 3 shows that anti-HRS antibodies inhibit tumor seeding and growth of B16-F10 Melanoma in the lung in an in vivo syngeneic mouse model more effectively than the combination of anti-PDL1 and anti-CTLA4 antibodies. Shown is the impact of IgG control antibody (circles); the combination of anti-PD-L1 and anti-CTLA4 antibodies (Squares) and the combination of an N-terminally directed (clone 13E9) and C-terminally directed antibody to HRS (clone 13C8) (Triangles) on the number of tumor nodules counted 18 days after intravenous tumor cell injection. Solid horizontal lines indicate group medians, dotted line indicates samples with nodules too numerous to count. For statistics, a value of 100 was assigned to these samples. Star indicates significance vs. IgG control via Kruskal-Wallis ANOVA, Dunn's post-hoc test. * p≤0.05.

FIGS. 4A-4B show a comparison of free HRS levels in naïve C57/Bl6 mice compared to mice into which B16-F10 melanoma cells have been introduced, and the impact of various treatments on free HRS levels measured using either an N-terminal, or full length specific ELISA assay. FIG. 4A shows the impact of IgG control antibody, the combination of anti-PD-1 and anti-CTL4 antibodies and the combination of an N-terminally directed (clone 13E9) and C-terminally directed antibody (clone 13C8) on free HRS levels in a melanoma solid tumor study. FIG. 4B shows the impact of the same treatments in a melanoma lung metastasis model. Dotted line indicates Lower Limit Of Quantification (LLOQ).

FIGS. 5A-5B show the PK characteristics of the anti-HRS antibody clone 13E9 (circles), and 13C8 (squares) in C57/Bl6 mice, administered IV (5A) or IP (5B).

FIG. 6 shows that an N-terminally-Directed anti-HRS Antibody (light squares) Slows Tumor Growth more effectively than the combination of anti-PD-L1 and anti-CTLA4 antibodies (dark triangles) in the B16-F10 synergic mouse model. Stars indicate significance, via 2-way ANOVA, Dunnett's post-hoc test. * p≤0.05, ** p<0.01, *** p<0.001.

FIGS. 7A-7H shows that anti-HRS antibodies cause the regression of 4T1 Tumors (a model of triple negative (ER, PR, HER2 negative) breast cancer) in a mouse syngeneic mouse model, and provides a memory response conferring resistance to re-inoculated tumor cells. FIG. 7A shows the impact of treatment with control mouse IgG on tumor volume with time, 7B shows the impact of treatment with mouse anti-PD-1 (αmPD-1) antibody on tumor volume with time, 7C shows the impact of treatment with mouse anti-PD-L1 (αmPD-L1) antibody on tumor volume with time, 7D shows the impact of treatment with mouse anti-HRS antibody 13E9 on tumor volume with time, 7E shows the impact of treatment with mouse anti-HRS antibody 13E9 in combination with a mouse anti-PD-1 (αmPD-1) antibody on tumor volume with time, 7F shows the impact of treatment with mouse anti-HRS antibody 13E9 in combination with a mouse anti-PD-L1 (αmPD-L1) antibody on tumor volume with time, 7G shows the results of challenge with tumor in previously naïve control mice, age matched to the other study animals, 7H shows the results of re-challenge with tumor 55 days after the last treatment with antibody in mice treated with mouse anti-HRS antibody 13E9 in combination with a mouse anti-PD-L1 (αmPD-L1) antibody on Days −1, 6 and 13. ↓ indicates treatment with antibodies “Tumor” indicates tumor inoculations.

FIG. 8 shows that human tumors secrete HRS after implantation into an immunocompromised mouse model. The graph shows the results of measuring human HRS via a species specific ELISA, in the serum in an immunocompromised mouse model (nu/nu), implanted with human tumor cells, in Naïve, control matrigel implanted mice, and after implantation of 2×10⁶ human A549 lung cancer cells, or 10×10⁶ human A549 lung cancer cells. Dotted line indicates Lower Limit Of Quantification (LLOQ).

FIG. 9 shows that mouse HRS levels are not significantly increased in response to a human xenograft. The figure shows the results of measuring mouse HRS levels via a species specific ELISA, in an immunocompromised mouse model (nu/nu) implanted with human tumor cells, in Naïve, control matrigel implanted mice, and after implantation of 2×10⁶ human A549 lung cancer cells, or 10×10⁶ human A549 lung cancer cells. Dotted line indicates Lower Limit Of Quantification (LLOQ).

FIG. 10 shows that human HRS levels correlate with tumor volume. The figure shows the results of measuring human HRS levels via a species specific ELISA, in an immunocompromised mouse model (nu/nu), implanted with human tumor cells, i.e., implantation of 2×10⁶ human A549 lung cancer cells, or 10×10⁶ human A549 lung cancer cells in animals with varying tumor volumes.

FIGS. 11A-11F show that the combination of anti-PD-L1 and anti-HRS antibodies synergistically inhibit tumor growth in the CT26 tumor model more effectively than either antibody alone. FIG. 11A shows the impact of treatment with control mouse IgG on tumor volume with time, 11B shows the impact of treatment with mouse anti-PD-1 (αmPD-1) antibody on tumor volume with time, 11C shows the impact of treatment with mouse anti-PD-L1 (αmPD-L1) antibody on tumor volume with time, 11D shows the impact of treatment with mouse anti-HRS antibody 13E9 on tumor volume with time, 11E shows the impact of treatment with mouse anti-HRS antibody 13E9 in combination with a mouse anti-PD-1 (αmPD-1) antibody on tumor volume with time, 11F shows the impact of treatment with mouse anti-HRS antibody 13E9 in combination with a mouse anti-PD-L1 (αmPD-L1) antibody on tumor volume with time. ↓ indicates treatment with antibodies.

FIGS. 12A-12H show that the combination of an anti-PD-L1 antibody and anti-HRS antibody synergistically inhibits tumor growth more effectively than either antibody alone, when administered starting 3 days after B16F10 melanoma tumor implantation. FIG. 12A shows the impact of treatment with control mouse IgG on tumor volume with time, 12B shows the impact of treatment with mouse anti-HRS antibody 13C8 on tumor volume with time, 12C shows the impact of treatment with mouse anti-HRS antibody 13E9 on tumor volume with time, 12D shows the impact of treatment with mouse anti-HRS antibody 13E9 in combination with anti-HRS antibody 13C8 on tumor volume with time, 12E shows the impact of treatment with mouse anti-PD-L1 (αmPD-1L) antibody on tumor volume with time, 12F shows the impact of treatment with mouse anti-HRS antibody 13C8 in combination with a mouse anti-PD-L1 antibody on tumor volume with time, 12G shows the impact of treatment with mouse anti-HRS antibody 13E9 in combination with a mouse anti-PD-L1 antibody on tumor volume with time, 12H shows the impact of treatment with mouse anti-PD-1 antibody in combination with a mouse anti-PD-L1 antibody on tumor volume with time. ↓ indicates treatment with antibodies.

FIGS. 13A-13D show that the combination of anti-PD-1 and anti-HRS antibodies synergistically inhibit tumor growth in the 4T1 breast cancer model system more effectively than either antibody alone. FIG. 13A shows the impact of treatment with control mouse IgG on tumor volume with time, 13B shows the impact of treatment with anti-mouse-PD-1 (αmPD-1) antibody on tumor volume with time, 13C shows the impact of treatment with mouse anti-HRS antibody 13E9 on tumor volume with time, 13D shows the impact of treatment with mouse anti-HRS antibody 13E9 in combination with an anti-mouse-PD-1 (αmPD-1) antibody on tumor volume with time. Upward ticks indicate days on which antibodies were administered.

FIGS. 14A-14I show that the combination of anti-PD-L1 or anti-PD-1 and anti-HRS antibodies tend to inhibit tumor growth in the Pan02 pancreatic cancer model more effectively than any antibody alone. FIG. 14A shows the impact of treatment with control mouse IgG on tumor volume with time, 14B shows the impact of treatment with mouse anti-HRS antibody 13C8 on tumor volume with time, 14C shows the impact of treatment with mouse anti-HRS antibody 13E9 on tumor volume with time, 14D shows the impact of treatment with anti-mouse PD-L1 (αmPD-1L) antibody on tumor volume with time, 14E shows the impact of treatment with anti-mouse PD-L1 (αmPD-L1) antibody in combination with mouse anti-HRS antibody 13C8 on tumor volume with time, 14F shows the impact of treatment with anti-mouse PD-L1 (αmPD-L1) antibody in combination with mouse anti-HRS antibody 13E9 on tumor volume with time, 14G shows the impact of treatment with anti-mouse PD-1 (αmPD-1) on tumor volume with time, 14H shows the impact of treatment with anti-mouse PD-1 (αmPD-1) antibody in combination with mouse anti-HRS antibody 13C8 on tumor volume with time, 14I shows the impact of treatment with anti-mouse PD-1 (αmPD-1) antibody in combination with mouse anti-HRS antibody 13E9 on tumor volume with time. Antibodies were administered the day before tumor cell inoculation and at weekly intervals for a total of 3 doses (Study Days −1, 6 and 13).

FIGS. 15A-15B show that the combination of indoleamine 2, 3-dioxygenase-1 (IDO) inhibition and anti-HRS antibody ATYR13E9 can regress tumors in CT26 colon cancer model more effectively than either alone. FIG. 15A shows the impact of treatment with control mouse IgG plus oral vehicle on tumor volume with time, 15B shows the impact of treatment with mouse anti-HRS antibody 13E9 plus oral vehicle on tumor volume with time.

FIGS. 16A-16B also show that the combination of IDO inhibition and anti-HRS antibody ATYR13E9 can regress tumors in CT26 colon cancer model more effectively than either alone. FIG. 16A shows the impact of treatment with control mouse IgG1 plus oral indoleamine 2, 3-dioxygenase-1 inhibitor (IDOi) on tumor volume with time, and 16B shows the impact of treatment with mouse anti-HRS antibody 13E9 plus oral IDOi on tumor volume with time. ↓ indicates treatment with antibodies (twice weekly for three weeks). IDOi was administered twice daily during the 3 week treatment period

FIGS. 17A-17C confirm the depletion of immune cells targeted in the example that shows the anti-cancer effects of anti-HRS antibodies depends on the presence of CD8+ T cells and NK1.1+ natural killer cells. FIG. 17A shows the impact of treatment with anti-CD4 antibody on circulating CD4+ T cells, 17B shows the impact of treatment with anti-CD4 antibody on circulating C8+ T cells, 17C shows the impact of treatment with anti-NK1.1 antibody on circulating NK1.1+ natural killer (NK) cells. Depletion antibodies were administered the day before tumor cell inoculation and at twice weekly intervals for a total of 5 doses (Study Days −1, 3, 6, 10, and 13).

FIGS. 18A-18E shows that anti-cancer effects of anti-HRS antibodies depends on presence of CD8+ T cells and NK1.1+ natural killer cells. FIG. 18A shows the impact of treatment with mouse anti-HRS antibodies ATYR13E9 and ATYR13C8 on tumor volume with time, 18B shows the impact of treatment with mouse anti-HRS antibodies ATYR13E9 and ATYR13C8 on tumor volume with time in mice depleted of CD8+ T cells, 18C shows the impact of treatment with mouse anti-HRS antibodies ATYR13E9 and ATYR13C8 on tumor volume with time in mice depleted of CD4+ T cells, 18D shows the impact of treatment with mouse anti-HRS antibodies ATYR13E9 and ATYR13C8 on tumor volume with time in mice depleted of NK1.1+ natural killer cells, 18E shows means±SEM of the treatment groups and results of statistical analysis (two-way ANOVA followed by Dunnet's post hoc test; **p<0.01, ***p<0.001, ****p<0.00010). Anti-HRS antibodies were administered the day before test tumor cell inoculation and at weekly intervals for a total of 3 doses (Study Days −1, 6 and 13); Depletion antibodies were administered the day before tumor cell inoculation and at twice weekly intervals for a total of 5 doses (Study Days −1, 3, 6, 10, and 13).

FIGS. 19A-19E show the evaluation of anti-tumor activity of test compounds on B16F10 mouse melanoma in C57bl/6 mice. FIG. 19A shows the impact of treatment with control mouse IgG on tumor volume with time, 19B shows the impact of treatment with mouse anti-PD-L1 antibody on tumor volume with time, 19C shows the impact of treatment with mouse anti-HRS antibody 13E9 on tumor volume with time, 19D shows the impact of treatment with humanized anti-HRS antibody KL31-600 on tumor volume with time, 19E shows the impact of treatment with anti-mouse PD-L1 (αmPD-L1) or anti-HRS antibodies on tumor volume measured on Day 20, the last day all animals were on study (left to right, IgG1, α-mPD-L1, 13E9, KL31-600). *p<0.05, ** p<0.01, 1 way ANOVA followed by Dunnett's post hoc test. Antibodies were administered the day before test tumor cell inoculation and at weekly intervals for a total of 3 doses (Study Days −1, 6 and 13).

FIGS. 20A-20F show that anti-HRS antibodies inhibit tumor growth and enhance tumor growth inhibition in combination with PD-L1 pathway blockade in the CT26 tumor model. FIG. 20A shows the change in tumor volume with time in untreated animals, 20B shows the impact of treatment with mouse anti-HRS antibody 13E9 on tumor volume with time, 20C shows the impact of treatment with humanized anti-HRS antibody KL31-241 on tumor volume with time, 20D shows the impact of treatment with anti-mouse PD-L1 (αmPD-L1) on tumor volume with time, 20E shows the impact of treatment with anti-mouse PD-L1 (αmPD-L1) antibody in combination with mouse anti-HRS antibody 13E9 on tumor volume with time, 20F shows the impact of treatment with anti-mouse PD-L1 (αmPD-L1) antibody in combination with humanized anti-HRS antibody KL31-241 on tumor volume with time. Antibodies were administered the day before test tumor cell inoculation and at weekly intervals for a total of 3 doses (Study Days −1, 6 and 13).

FIGS. 21A-21F show that in contrast to anti-PD-L1 antibodies, anti-HRS antibodies do not precipitate Type 1 Diabetes in female NOD mice. FIG. 21A shows the impact of rIgG2b (control for anti-mouse PD-L1) treatment on glucose measurements with time, 21B shows the impact of treatment with mouse IgG1 (control for 13E9) on glucose measurements with time, 21C shows the impact of treatment with human IgG1 (control for KL31-241) on glucose measurements with time, 21D shows the impact of treatment with anti-mouse PD-L1 (αmPD-L1) on glucose measurements with time, 21E shows the impact of treatment with mouse anti-HRS antibody 13E9 on glucose measurements with time, 21F shows the impact of treatment with humanized anti-HRS antibody KL31-241 on glucose measurements with time. Antibodies were administered to 11 week old mice on Study Days 0, 4, 7 and 11.

FIG. 22 shows binding of human NRP2 to Fc-HRS(2-60) on an SPR chip. 50 nM of NRP2 (solid black line), NRP1 (solid gray line) and mouse Plexin A1 (dotted line) were flowed as analytes over an SPR chip coated with immobilized Fc-HRS(2-60).

FIGS. 23A-23B shows binding of NRP2 from human, mouse, and rat to Fc-HRS(2-60) and not a truncated form of Fc-HRS(2-11). 50 nM of human NRP2 (solid black line), mouse NRP2 (dashed line), rat NRP2 (solid gray line), or NRP1 (dotted line) were flowed as analytes over an SPR chip coated with immobilized full length Fc-HRS(2-60) (23A), or a truncated form of Fc-HRS(2-11) missing 49 amino acids at the C-terminus (23B).

FIGS. 24A-24D show binding of human NRP2 to Fc-HRS(2-60) and t-RNA synthetases comprising domains that share homology with the WHEP domain of Fc-HRS(2-60). 20 nM of NRP2 was flowed as an analyte over SPR chip surfaces coated with immobilized Fc-HRS(2-60) (24A), GARS Fc-WHEP (24B), MARS Fc-WHEP (24C), or WARS WHEP (24D).

FIGS. 25A-25B show the binding of human NRP2 to Fc-HRS(2-60) on an SPR chip coated with immobilized Fc-HRS(2-60) in the presence and absence of divalent cations. The running buffer in this experiment was 50 mM HEPES, 300 mM NaCl, 0.005% Tween20, pH 7.4. For each analyte, 20 nM NRP2 was prepared in running buffer supplemented with 5 mM of either CaCl₂, EDTA (25A) or MgCl₂, MgCl₂+CaCl₂ or ZnCl₂ (25B).

FIGS. 26A-26B show binding of a pre-formed complex of Fc-HRS(2-60) and NRP2 to 4D4 monoclonal antibody but not to the 1C8 monoclonal antibody. Monoclonal antibodies against Fc-HRS(2-60) (monoclonal antibody clones 1C8 (26A) and 4D4 (26B)) were immobilized on an SPR chip. Analytes consisted of 200 nM NRP2 (dotted line), 100 nM Fc-HRS(2-60) (solid black line), a mixture of 100 nM Fc-HRS(2-60) and 200 nM NRP2 (solid gray line), or a mixture of 100 nM Fc-HRS(2-60) and 200 nM 1C8 mAb (dashed line).

FIGS. 27A-27D show binding of NRP2 to Fc-HRS(2-60) captured by some monoclonal antibodies against Fc-HRS(2-60) but not others. Monoclonal antibodies against Fc-HRS(2-60) (Monoclonal antibody clones 12H6 (27A), 1C8 (27B), 4D4 (27C) and 13E9 (27D) were immobilized on an SPR chip. Co-injections were then carried out where one analyte is injected, immediately followed by a second analyte. Timing of the two injections is indicated by arrowheads. In each of the panels above, 2000 nM Fc-HRS(2-60) was injected as the first analyte to saturate the antibody surfaces, followed by either additional Fc-HRS(2-60) (solid gray line), or 200 nM NRP2 (solid black line). To rule out non-specific binding of NRP2 to the antibody surfaces, co-injection of buffer followed by 200 nM NRP2 were also performed (dotted line).

FIGS. 28A-28B show dose-dependent binding of Fc-HRS (2-60) to cells expressing a NRP2a-GFP fusion protein. Quantification of the staining intensity (28A) and staining intensity CV (28B) of Fc-HRS (2-60)/anti-Fc-PE complex on HEK293T cells overexpressing NRP2v2-GFP. Intensity values are from cells gated on high NRP2 expression (GFP Bright). Fc-HRS (2-60) was titrated in 2 fold steps and then combined with 87.5 nM of anti-Fc-PE. As a control for specificity, 175 nM Fc-HRS (2-11)/anti-Fc-PE was included.

FIG. 29 shows binding inhibition of Fc-HRS (2-60) to cells expressing a NRP2a-GFP fusion protein in the presence of anti-HRS antibody clone 1C8. Quantification of the staining intensity of Fc-HRS (2-60)/anti-Fc-PE complex pre-incubated with either an isotype antibody control or anti-HRS (WHEP) clone 1C8 on HEK293T cells overexpressing NRP2v2-GFP. Intensity values are from cells gated on high NRP2 expression (GFP Bright). 175 nM of Fc-HRS (2-60)/anti-Fc-PE was used. As a control for specificity, 175 nM Fc-HRS (2-11)/anti-Fc-PE was included.

FIGS. 30A-30B show that anti-HRS antibodies from the KL31 series blocked binding of Fc-HRS(2-60) to NRP2 in a concentration-dependent manner, whereas other antibodies of the AB04 and AB13 series did not demonstrate significant blocking characteristics in this assay. Quantification of the staining of stably expressing Expi293-NRP2 cells with biotinylated Fc-HRS-streptavidin-PE using flow cytometry in the presence of various concentrations of anti-HRS antibodies. Data are from two experiments using different antibodies. FIG. 30A shows control human IgG1 (filled circles), KL31-467 (filled triangles), KL31-356 (partially filled triangles), mouse clone 13C8 (crosses), and 30B shows control human IgG1 (filled circles), AB04-425 (open triangles), AB13-288 (partially filled squares), and KL31-478 (filled triangles), which are shown as the percentage of streptavidin-PE+/NRP2+ cells in the viable singlet gate.

FIGS. 31A-31B show binding inhibition of Fc-HRS (2-60) to cells expressing a NRP2a-GFP fusion protein in the presence of VEGF-C. Quantification of the staining intensity of Fc-HRS (2-60)/anti-Fc-PE complex pre-incubated with different doses of VEGF-C on HEK293T cells overexpressing NRP2v2-GFP. Intensity values are from cells gated on high NRP2 expression (GFP Bright). 175 nM of Fc-HRS (2-60)/anti-Fc-PE was used. As a control for specificity, 175 nM Fc-HRS (2-11)/anti-Fc-PE was included.

FIG. 32 shows quantification of circulating NRP2 levels in serum and plasma from normal healthy donors. Normal healthy volunteer (n=72) serum and plasma was isolated and quantified for circulating levels of NRP-2. Serum (black circles) and plasma (open squares) samples were tested in an ELISA specific for human NRP-2. Mean levels for serum (16.3 pM) and plasma (15.6 pM) were shown for all 72 samples. The limit of quantification for the NRP2 ELISA was 1.5 pM.

FIG. 33 shows a comparison of circulating HRS and NRP2 levels. Serum HRS (black circles) levels show a broad range in circulation within 72 normal healthy volunteers tested. Matching serum NRP2 levels from the identical donors were overlaid on the same axes. Donors with low HRS levels show low to undetectable levels of soluble NRP2 (Limit of quantification=1.5 pM). Those donors with elevated HRS levels generally have corresponding increased levels of circulating NRP2.

FIG. 34 shows HRS N-terminal interference in human serum from healthy donors. Normal serum from healthy donors was assayed in two separate HARS ELISAs. Samples were assayed in an ELISA to detect full length HARS (HARS_FL; black circles) as well as an ELISA directed against specifically the N-terminus (HARS_NT; open squares). The lack of correlation between these two ELISAs, as full length HARS levels increased, is referred to as N-terminal interference and may represent the presence of a cofactor, binding partner or soluble receptor to HRS.

FIG. 35 shows a correlation between HARS N-terminal assay interference and soluble NRP2 levels. Normal healthy serum was analyzed for differences in detection with two HARS ELISAs and compared to circulating NRP2 levels. The difference in levels detected between the full length HARS ELISA and the N-terminal HARS ELISA was termed HARS N-terminal Interference Units. These interference units were plotted versus soluble NRP2 levels. The results show a relationship between increased N-terminal interference and soluble NRP2 in normal serum.

FIG. 36 shows detection of an endogenous HRS & NRP2 soluble complex. Serum samples from normal healthy donors were analyzed in multiple HRS & NRP-2 complex ELISAs. These assay formats utilized capture of circulating HRS (HARS_NT or HARS_CT) and detection with an NRP2 monoclonal antibody. Similarly the reverse format was also used whereby circulating NRP2 was captured and detection was observed with anti-HRS antibodies. In both formats, signals were elevated in the high interference samples as compared to low interference serum samples.

FIG. 37 shows that complexed HRS and NRP2 in high interference samples blocks detection with a site-specific HRS antibody. Serum from low and high HRS N-terminal interference samples was assayed in a HRS and NRP2 complex ELISA. Serum samples were captured with an NRP2 monoclonal antibody and detected with either of two unique HRS N-terminal monoclonal antibodies. Samples with high interference showed complex formation when detected with HRS_NT (black bars) but this signal was completely blocked with an N-terminal anti-HRS antibody (HRS blocking antibody; gray bars).

FIG. 38 shows an elevation of HRS baseline levels in all (15/15) cancer types tested relative to normal healthy controls.

FIGS. 39A-39E show the evaluation of anti-tumor activity of test compounds on B16F10 mouse melanoma in C57bl/6 mice. FIG. 39A shows the impact of treatment with control mouse IgG on tumor volume with time, 39B shows the impact of treatment with mouse anti-PD-L1 antibody on tumor volume with time, 39C shows the impact of treatment with mouse anti-HARS antibody 13E9 on tumor volume with time, 39D shows the impact of treatment with human anti-HARS antibody AB04 on tumor volume with time, 9E shows the impact of treatment with anti-mouse PD-L1 or anti-HARS antibodies on tumor volume measured on Day 20 (left to right, IgG1, anti-PD-L1, 13E9, AB04, KL31) the last day all animals were on study. Antibodies were administered the day before test tumor cell inoculation and at weekly intervals for a total of 3 doses (Study Days −1, 6 and 13).

FIGS. 40A-40F show that anti-HARS antibodies inhibit tumor growth and enhance tumor growth inhibition in combination with PD-L1 pathway blockade in the CT26 tumor model. FIG. 40A shows the change in tumor volume with time in untreated animals, 40B shows the impact of treatment with mouse anti-HARS antibody 13E9 on tumor volume with time, 40C shows the impact of treatment with human anti-HARS antibody AB13 on tumor volume with time, 40D shows the impact of treatment with anti-mouse PD-L1 on tumor volume with time, 40E shows the impact of treatment with anti-mouse PD-L1 antibody in combination with mouse anti-HARS antibody 13E9 on tumor volume with time, 40F shows the impact of treatment with anti-mouse PD-L1 antibody in combination with human anti-HARS antibody AB13 on tumor volume with time. Antibodies were administered the day before test tumor cell inoculation and at weekly intervals for a total of 3 doses (Study Days −1, 6 and 13).

### DETAILED DESCRIPTION

Unless defined otherwise, all technical and scientific terms used herein have the same meaning as commonly understood by those of ordinary skill in the art to which the disclosure belongs. Although any methods, materials, compositions, reagents, cells, similar or equivalent similar or equivalent to those described herein can be used in the practice or testing of the subject matter of the present disclosure, preferred methods and materials are described. All publications and references, including but not limited to patents and patent applications, cited in this specification are herein incorporated by reference in their entirety as if each individual publication or reference were specifically and individually indicated to be incorporated by reference herein as being fully set forth. Any patent application to which this application claims priority is also incorporated by reference herein in its entirety in the manner described above for publications and references.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

For the purposes of the present disclosure, the following terms are defined below.

The articles “a” and “an” are used herein to refer to one or to more than one (i.e., to at least one) of the grammatical object of the article. By way of example, “an element” includes “one element”, “one or more elements” and/or “at least one element”.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

The term “antigen” refers to a molecule or a portion of a molecule capable of being bound by a selective binding agent, such as an antibody, and additionally capable of being used in an animal to produce antibodies capable of binding to an epitope of that antigen. An antigen may have one or more epitopes. As used herein, the term “antigen” includes substances that are capable, under appropriate conditions, of inducing an immune response to the substance and of reacting with the products of the immune response. For example, an antigen can be recognized by antibodies (humoral immune response) or sensitized T-lymphocytes (T helper or cell-mediated immune response), or both. Antigens can be soluble substances, such as toxins and foreign proteins, or particulates, such as bacteria and tissue cells; however, only the portion of the protein or polysaccharide molecule known as the antigenic determinant (epitopes) combines with the antibody or a specific receptor on a lymphocyte. More broadly, the term “antigen” includes any substance to which an antibody binds, or for which antibodies are desired, regardless of whether the substance is immunogenic. For such antigens, antibodies can be identified by recombinant methods, independently of any immune response.

An “antagonist” refers to biological structure or chemical agent that interferes with or otherwise reduces the physiological action of another agent or molecule. In some instances, the antagonist specifically binds to the other agent or molecule. Included are full and partial antagonists.

An “agonist” refers to biological structure or chemical agent that increases or enhances the physiological action of another agent or molecule. In some instances, the agonist specifically binds to the other agent or molecule. Included are full and partial agonists.

The term “anergy” refers to the functional inactivation of a T-cell, or B-cell response to re-stimulation by antigen.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

As used herein, the term “antibody” encompasses not only intact polyclonal or monoclonal antibodies, but also fragments thereof (such as dAb, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, and any other modified configuration of the immunoglobulin molecule that comprises an antigen-binding site or fragment (epitope recognition site) of the required specificity. Certain features and characteristics of antibodies (and antigen-binding fragments thereof) are described in greater detail herein.

An antibody or antigen-binding fragment can be of essentially any type. As is well known in the art, an antibody is an immunoglobulin molecule capable of specific binding to a target, such as an immune checkpoint molecule, through at least one epitope recognition site, located in the variable region of the immunoglobulin molecule.

The term “antigen-binding fragment” as used herein refers to a polypeptide fragment that contains at least one CDR of an immunoglobulin heavy and/or light chain that binds to the antigen of interest. In this regard, an antigen-binding fragment of the herein described antibodies may comprise 1, 2, 3, 4, 5, or all 6 CDRs of a V_{H} and V_{L} sequence from antibodies that bind to a target molecule.

The binding properties of antibodies and antigen-binding fragments thereof can be quantified using methods well known in the art (see Davies et al., Annual Rev. Biochem. 59:439-473, 1990). In some embodiments, an antibody or antigen-binding fragment thereof specifically binds to a target molecule, for example, an HRS polypeptide or an epitope or complex thereof, with an equilibrium dissociation constant that is about or ranges from about ≤10−7 to about 10−8 M. In some embodiments, the equilibrium dissociation constant is about or ranges from about ≤10−9 M to about ≤10−10 M. In certain illustrative embodiments, an antibody or antigen-binding fragment thereof has an affinity (Kd) for a target molecule (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

A molecule such as a polypeptide or antibody is said to exhibit “specific binding” or “preferential binding” if it reacts or associates more frequently, more rapidly, with greater duration and/or with greater affinity with a particular cell, substance, or particular epitope than it does with alternative cells or substances, or epitopes. An antibody “specifically binds” or “preferentially binds” to a target molecule or epitope if it binds with greater affinity, avidity, more readily, and/or with greater duration than it binds to other substances or epitopes, for example, by a statistically significant amount. Typically one member of the pair of molecules that exhibit specific binding has an area on its surface, or a cavity, which specifically binds to and is therefore complementary to a particular spatial and/or polar organization of the other member of the pair of molecules. Thus, the members of the pair have the property of binding specifically to each other. For instance, an antibody that specifically or preferentially binds to a specific epitope is an antibody that binds that specific epitope with greater affinity, avidity, more readily, and/or with greater duration than it binds to other epitopes. It is also understood by reading this definition that, for example, an antibody (or moiety or epitope) that specifically or preferentially binds to a first target may or may not specifically or preferentially bind to a second target. The term is also applicable where, for example, an antibody is specific for a particular epitope which is carried by a number of antigens, in which case the specific binding member carrying the antigen-binding fragment or domain will be able to bind to the various antigens carrying the epitope; for example, it may be cross reactive to a number of different forms of a target antigen from multiple species that share a common epitope Immunological binding generally refers to the non-covalent interactions of the type which occur between an immunoglobulin molecule and an antigen for which the immunoglobulin is specific, for example by way of illustration and not limitation, as a result of electrostatic, ionic, hydrophilic and/or hydrophobic attractions or repulsion, steric forces, hydrogen bonding, van der Waals forces, and other interactions. The strength, or affinity of immunological binding interactions can be expressed in terms of the dissociation constant (Kd) of the interaction, wherein a smaller Kd represents a greater affinity.

Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kon) and the “off rate constant” (Koff) can be determined by calculation of the concentrations and the actual rates of association and dissociation. The ratio of Koff/Kon enables cancellation of all parameters not related to affinity, and is thus equal to the dissociation constant Kd. As used herein, the term “affinity” includes the equilibrium constant for the reversible binding of two agents and is expressed as Kd. Affinity of a binding protein to a ligand such as affinity of an antibody for an epitope can be, for example, from about 100 nanomolar (nM) to about 0.1 nM, from about 100 nM to about 1 picomolar (pM), or from about 100 nM to about 1 femtomolar (fM). As used herein, the term “avidity” refers to the resistance of a complex of two or more agents to dissociation after dilution Antibodies may be prepared by any of a variety of techniques known to those of ordinary skill in the art. See, e.g., Harlow and Lane, Antibodies: A Laboratory Manual, Cold Spring Harbor Laboratory, 1988. Monoclonal antibodies specific for a polypeptide of interest may be prepared, for example, using the technique of Kohler and Milstein, Eur. J. Immunol. 6:511-519, 1976, and improvements thereto. Also included are methods that utilize transgenic animals such as mice to express human antibodies. See, e.g., Neuberger et al., Nature Biotechnology 14:826, 1996; Lonberg et al., Handbook of Experimental Pharmacology 113:49-101, 1994; and Lonberg et al., Internal Review of Immunology 13:65-93, 1995. Particular examples include the VELOCIMMUNE® platform by REGENEREX® (see, e.g., U.S. Pat. No. 6,596,541).

Antibodies can also be generated or identified by the use of phage display or yeast display libraries (see, e.g., U.S. Pat. No. 7,244,592; Chao et al., Nature Protocols. 1:755-768, 2006). Non-limiting examples of available libraries include cloned or synthetic libraries, such as the Human Combinatorial Antibody Library (HuCAL), in which the structural diversity of the human antibody repertoire is represented by seven heavy chain and seven light chain variable region genes. The combination of these genes gives rise to 49 frameworks in the master library. By superimposing highly variable genetic cassettes (CDRs=complementarity determining regions) on these frameworks, the vast human antibody repertoire can be reproduced. Also included are human libraries designed with human-donor-sourced fragments encoding a light-chain variable region, a heavy-chain CDR-3, synthetic DNA encoding diversity in heavy-chain CDR-1, and synthetic DNA encoding diversity in heavy-chain CDR-2. Other libraries suitable for use will be apparent to persons skilled in the art.

In certain embodiments, antibodies and antigen-binding fragments thereof as described herein include a heavy chain and a light chain CDR set, respectively interposed between a heavy chain and a light chain framework region (FR) set which provide support to the CDRs and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRs. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

The structures and locations of immunoglobulin variable domains may be determined by reference to Kabat, E. A. et al., Sequences of Proteins of Immunological Interest. 4th Edition. US Department of Health and Human Services. 1987, and updates thereof.

Also include are “monoclonal” antibodies, which refer to a homogeneous antibody population wherein the monoclonal antibody is comprised of amino acids (naturally occurring and non-naturally occurring) that are involved in the selective binding of an epitope. Monoclonal antibodies are highly specific, being directed against a single epitope. The term “monoclonal antibody” encompasses not only intact monoclonal antibodies and full-length monoclonal antibodies, but also fragments thereof (such as Fab, Fab′, F(ab′)2, Fv), single chain (ScFv), variants thereof, fusion proteins comprising an antigen-binding portion, humanized monoclonal antibodies, chimeric monoclonal antibodies, and any other modified configuration of the immunoglobulin molecule that comprises an antigen-binding fragment (epitope recognition site) of the required specificity and the ability to bind to an epitope. It is not intended to be limited as regards the source of the antibody or the manner in which it is made (e.g., by hybridoma, phage selection, recombinant expression, transgenic animals). The term includes whole immunoglobulins as well as the fragments etc. described above under the definition of “antibody.”

The proteolytic enzyme papain preferentially cleaves IgG molecules to yield several fragments, two of which (the F(ab) fragments) each comprise a covalent heterodimer that includes an intact antigen-binding site. The enzyme pepsin is able to cleave IgG molecules to provide several fragments, including the F(ab′)2 fragment which comprises both antigen-binding sites. An Fv fragment for use according to certain embodiments of the present invention can be produced by preferential proteolytic cleavage of an IgM, and on rare occasions of an IgG or IgA immunoglobulin molecule. Fv fragments are, however, more commonly derived using recombinant techniques known in the art. The Fv fragment includes a non-covalent VH::VL heterodimer including an antigen-binding site which retains much of the antigen recognition and binding capabilities of the native antibody molecule. See Inbar et al., PNAS USA. 69:2659-2662, 1972; Hochman et al., Biochem. 15:2706-2710, 1976; and Ehrlich et al., Biochem. 19:4091-4096, 1980.

In certain embodiments, single chain Fv (scFV) antibodies are contemplated. For example, Kappa bodies (Ill et al., Prot. Eng. 10:949-57, 1997); minibodies (Martin et al., EMBO J 13:5305-9, 1994); diabodies (Holliger et al., PNAS 90: 6444-8, 1993); or Janusins (Traunecker et al., EMBO J 10: 3655-59, 1991; and Traunecker et al., Int. J. Cancer Suppl. 7:51-52, 1992), may be prepared using standard molecular biology techniques following the teachings of the present application with regard to selecting antibodies having the desired specificity.

A single chain Fv (scFv) polypeptide is a covalently linked VH::VL heterodimer which is expressed from a gene fusion including VH- and VL-encoding genes linked by a peptide-encoding linker. Huston et al. (PNAS USA. 85(16):5879-5883, 1988). A number of methods have been described to discern chemical structures for converting the naturally aggregated—but chemically separated—light and heavy polypeptide chains from an antibody V region into an scFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

In certain embodiments, the antibodies or antigen-binding fragments described herein are in the form of a “diabody.” Diabodies are multimers of polypeptides, each polypeptide comprising a first domain comprising a binding region of an immunoglobulin light chain and a second domain comprising a binding region of an immunoglobulin heavy chain, the two domains being linked (e.g. by a peptide linker) but unable to associate with each other to form an antigen-binding site: antigen-binding sites are formed by the association of the first domain of one polypeptide within the multimer with the second domain of another polypeptide within the multimer (WO94/13804). A dAb fragment of an antibody consists of a VH domain (Ward et al., Nature 341:544-546, 1989). Diabodies and other multivalent or multispecific fragments can be constructed, for example, by gene fusion (see WO94/13804; and Holliger et al., PNAS USA. 90:6444-6448, 1993)).

Minibodies comprising a scFv joined to a CH3 domain are also included (see Hu et al., Cancer Res. 56:3055-3061, 1996). See also Ward et al., Nature. 341:544-546, 1989; Bird et al., Science. 242:423-426, 1988; Huston et al., PNAS USA. 85:5879-5883, 1988); PCT/US92/09965; WO94/13804; and Reiter et al., Nature Biotech. 14:1239-1245, 1996.

Where bispecific antibodies are to be used, these may be conventional bispecific antibodies, which can be manufactured in a variety of ways (Holliger and Winter, Current Opinion Biotechnol. 4:446-449, 1993), e.g. prepared chemically or from hybrid hybridomas, or may be any of the bispecific antibody fragments mentioned above. Diabodies and scFv can be constructed without an Fc region, using only variable domains, potentially reducing the effects of anti-idiotypic reaction.

Bispecific diabodies, as opposed to bispecific whole antibodies, may also be particularly useful because they can be readily constructed and expressed in E. coli. Diabodies (and many other polypeptides such as antibody fragments) of appropriate binding specificities can be readily selected using phage display (WO94/13804) from libraries. If one arm of the diabody is to be kept constant, for instance, with a specificity directed against antigen X, then a library can be made where the other arm is varied and an antibody of appropriate specificity selected. Bispecific whole antibodies may be made by knobs-into-holes engineering (Ridgeway et al., Protein Eng., 9:616-621, 1996).

In certain embodiments, the antibodies or antigen-binding fragments described herein are in the form of a UniBody®. A UniBody® is an IgG4 antibody with the hinge region removed (see GenMab Utrecht, The Netherlands; see also, e.g., US20090226421). This antibody technology creates a stable, smaller antibody format with an anticipated longer therapeutic window than current small antibody formats. IgG4 antibodies are considered inert and thus do not interact with the immune system. Fully human IgG4 antibodies may be modified by eliminating the hinge region of the antibody to obtain half-molecule fragments having distinct stability properties relative to the corresponding intact IgG4 (GenMab, Utrecht). Halving the IgG4 molecule leaves only one area on the UniBody® that can bind to cognate antigens (e.g., disease targets) and the UniBody® therefore binds univalently to only one site on target cells. For certain cancer cell surface antigens, this univalent binding may not stimulate the cancer cells to grow as may be seen using bivalent antibodies having the same antigen specificity, and hence UniBody® technology may afford treatment options for some types of cancer that may be refractory to treatment with conventional antibodies. The small size of the UniBody® can be a great benefit when treating some forms of cancer, allowing for better distribution of the molecule over larger solid tumors and potentially increasing efficacy.

In certain embodiments, the antibodies and antigen-binding fragments described herein are in the form of a nanobody. Minibodies are encoded by single genes and are efficiently produced in almost all prokaryotic and eukaryotic hosts, for example, E. coli (see U.S. Pat. No. 6,765,087), molds (for example Aspergillus or Trichoderma) and yeast (for example Saccharomyces, Kluyvermyces, Hansenula or Pichia (see U.S. Pat. No. 6,838,254). The production process is scalable and multi-kilogram quantities of nanobodies have been produced. Nanobodies may be formulated as a ready-to-use solution having a long shelf life. The Nanoclone method (see WO 06/079372) is a proprietary method for generating Nanobodies against a desired target, based on automated high-throughput selection of B-cells.

In some embodiments, the antibodies or antigen-binding fragments described herein are in the form of an aptamer (see, e.g., Ellington et al., Nature. 346, 818-22, 1990; and Tuerk et al., Science. 249, 505-10, 1990, incorporated by reference). Examples of aptamers included nucleic acid aptamers (e.g., DNA aptamers, RNA aptamers) and peptide aptamers. Nucleic acid aptamers refer generally to nucleic acid species that have been engineered through repeated rounds of in vitro selection or equivalent method, such as SELEX (systematic evolution of ligands by exponential enrichment), to bind to various molecular targets such as small molecules, proteins, nucleic acids, and even cells, tissues and organisms. See, e.g., U.S. Pat. Nos. 6,376,190; and 6,387,620, incorporated by reference.

Peptide aptamers typically include a variable peptide loop attached at both ends to a protein scaffold, a double structural constraint that typically increases the binding affinity of the peptide aptamer to levels comparable to that of an antibody's (e.g., in the nanomolar range). In certain embodiments, the variable loop length may be composed of about 10-20 amino acids (including all integers in between), and the scaffold may include any protein that has good solubility and compacity properties. Certain exemplary embodiments utilize the bacterial protein Thioredoxin-A as a scaffold protein, the variable loop being inserted within the reducing active site (-Cys-Gly-Pro-Cys-loop in the wild protein), with the two cysteines lateral chains being able to form a disulfide bridge. Methods for identifying peptide aptamers are described, for example, in U.S. Application No. 2003/0108532, incorporated by reference. Peptide aptamer selection can be performed using different systems known in the art, including the yeast two-hybrid system.

In some embodiments, the antibodies or antigen-binding fragments described herein are in the form of an avimer. Avimers refer to multimeric binding proteins or peptides engineered using in vitro exon shuffling and phage display. Multiple binding domains are linked, resulting in greater affinity and specificity compared to single epitope immunoglobulin domains. See, e.g., Silverman et al., Nature Biotechnology. 23:1556-1561, 2005; U.S. Pat. No. 7,166,697; and U.S. Application Nos. 2004/0175756, 2005/0048512, 2005/0053973, 2005/0089932 and 2005/0221384, incorporated by reference.

In some embodiments, the antibodies or antigen-binding fragments described herein are in the form of an adnectin. Adnectins refer to a class of targeted biologics derived from human fibronectin, an abundant extracellular protein that naturally binds to other proteins. See, e.g., U.S. Application Nos. 2007/0082365; 2008/0139791; and 2008/0220049, incorporated by reference. Adnectins typically consists of a natural fibronectin backbone, as well as the multiple targeting domains of a specific portion of human fibronectin. The targeting domains can be engineered to enable an adnectin to specifically recognize an HRS polypeptide or an epitope thereof.

In some embodiments, the antibodies or antigen-binding fragments described herein are in the form of an anticalin. Anticalins refer to a class of antibody mimetics that are typically synthesized from human lipocalins, a family of binding proteins with a hypervariable loop region supported by a structurally rigid framework. See, e.g., U.S. Application No. 2006/0058510. Anticalins typically have a size of about 20 kDa. Anticalins can be characterized by a barrel structure formed by eight antiparallel β-strands (a stable β-barrel scaffold) that are pairwise connected by four peptide loops and an attached α-helix. In certain aspects, conformational deviations to achieve specific binding are made in the hypervariable loop region(s). See, e.g., Skerra, FEBS J. 275:2677-83, 2008, incorporated by reference.

In some embodiments, the antibodies or antigen-binding fragments described herein are in the form of a designed ankyrin repeat protein (DARPin). DARPins include a class of non-immunoglobulin proteins that can offer advantages over antibodies for target binding in drug discovery and drug development. Among other uses, DARPins are ideally suited for in vivo imaging or delivery of toxins or other therapeutic payloads because of their favorable molecular properties, including small size and high stability. The low-cost production in bacteria and the rapid generation of many target-specific DARPins make the DARPin approach useful for drug discovery. Additionally, DARPins can be easily generated in multispecific formats, offering the potential to target an effector DARPin to a specific organ or to target multiple receptors with one molecule composed of several DARPins. See, e.g., Stumpp et al., Curr Opin Drug Discov Devel. 10:153-159, 2007; U.S. Application No. 2009/0082274; and PCT/EP2001/10454, incorporated by reference.

Also included are heavy chain dimers, such as antibodies from camelids and sharks. Camelid and shark antibodies comprise a homodimeric pair of two chains of V-like and C-like domains (neither has a light chain). Since the VH region of a heavy chain dimer IgG in a camelid does not have to make hydrophobic interactions with a light chain, the region in the heavy chain that normally contacts a light chain is changed to hydrophilic amino acid residues in a camelid. VH domains of heavy-chain dimer IgGs are called VHH domains. Shark Ig-NARs comprise a homodimer of one variable domain (termed a V-NAR domain) and five C-like constant domains (C-NAR domains).

In camelids, the diversity of antibody repertoire is determined by the complementary determining regions (CDR) 1, 2, and 3 in the VH or VHH regions. The CDR3 in the camel VHH region is characterized by its relatively long length averaging 16 amino acids (Muyldermans et al., 1994, Protein Engineering 7(9): 1129). This is in contrast to CDR3 regions of antibodies of many other species. For example, the CDR3 of mouse VH has an average of 9 amino acids. Libraries of camelid-derived antibody variable regions, which maintain the in vivo diversity of the variable regions of a camelid, can be made by, for example, the methods disclosed in U.S. Patent Application Ser. No. 20050037421, published Feb. 17, 2005

In certain embodiments, the antibodies or antigen-binding fragments thereof are humanized. These embodiments refer to a chimeric molecule, generally prepared using recombinant techniques, having an antigen-binding site derived from an immunoglobulin from a non-human species and the remaining immunoglobulin structure of the molecule based upon the structure and/or sequence of a human immunoglobulin. The antigen-binding site may comprise either complete variable domains fused onto constant domains or only the CDRs grafted onto appropriate framework regions in the variable domains. Epitope binding sites may be wild type or modified by one or more amino acid substitutions. This eliminates the constant region as an immunogen in human individuals, but the possibility of an immune response to the foreign variable region remains (LoBuglio et al., PNAS USA 86:4220-4224, 1989; Queen et al., PNAS USA. 86:10029-10033, 1988; Riechmann et al., Nature. 332:323-327, 1988). Illustrative methods for humanization of antibodies include the methods described in U.S. Pat. No. 7,462,697.

Another approach focuses not only on providing human-derived constant regions, but modifying the variable regions as well so as to reshape them as closely as possible to human form. It is known that the variable regions of both heavy and light chains contain three complementarity-determining regions (CDRs) which vary in response to the epitopes in question and determine binding capability, flanked by four framework regions (FRs) which are relatively conserved in a given species and which putatively provide a scaffolding for the CDRs. When nonhuman antibodies are prepared with respect to a particular epitope, the variable regions can be “reshaped” or “humanized” by grafting CDRs derived from nonhuman antibody on the FRs present in the human antibody to be modified. Application of this approach to various antibodies has been reported by Sato et al., Cancer Res. 53:851-856, 1993; Riechmann et al., Nature 332:323-327, 1988; Verhoeyen et al., Science 239:1534-1536, 1988; Kettleborough et al., Protein Engineering. 4:773-3783, 1991; Maeda et al., Human Antibodies Hybridoma 2:124-134, 1991; Gorman et al., PNAS USA. 88:4181-4185, 1991; Tempest et al., Bio/Technology 9:266-271, 1991; Co et al., PNAS USA. 88:2869-2873, 1991; Carter et al., PNAS USA. 89:4285-4289, 1992; and Co et al., J Immunol. 148:1149-1154, 1992. In some embodiments, humanized antibodies preserve all CDR sequences (for example, a humanized mouse antibody which contains all six CDRs from the mouse antibodies). In other embodiments, humanized antibodies have one or more CDRs (one, two, three, four, five, six) which are altered with respect to the original antibody, which are also termed one or more CDRs “derived from” one or more CDRs from the original antibody.

In certain embodiments, the antibodies are “chimeric” antibodies. In this regard, a chimeric antibody is comprised of an antigen-binding fragment of an antibody operably linked or otherwise fused to a heterologous Fc portion of a different antibody. In certain embodiments, the Fc domain or heterologous Fc domain is of human origin. In certain embodiments, the Fc domain or heterologous Fc domain is of mouse origin. In other embodiments, the heterologous Fc domain may be from a different Ig class from the parent antibody, including IgA (including subclasses IgA1 and IgA2), IgD, IgE, IgG (including subclasses IgG1, IgG2, IgG3, and IgG4), and IgM. In further embodiments, the heterologous Fc domain may be comprised of CH2 and CH3 domains from one or more of the different Ig classes. As noted above with regard to humanized antibodies, the antigen-binding fragment of a chimeric antibody may comprise only one or more of the CDRs of the antibodies described herein (e.g., 1, 2, 3, 4, 5, or 6 CDRs of the antibodies described herein), or may comprise an entire variable domain (VL, VH or both).

As used herein, a subject “at risk” of developing a disease, or adverse reaction may or may not have detectable disease, or symptoms of disease, and may or may not have displayed detectable disease or symptoms of disease prior to the treatment methods described herein. “At risk” denotes that a subject has one or more risk factors, which are measurable parameters that correlate with development of a disease, as described herein and known in the art. A subject having one or more of these risk factors has a higher probability of developing disease, or an adverse reaction than a subject without one or more of these risk factor(s).

“Biocompatible” refers to materials or compounds which are generally not injurious to biological functions of a cell or subject and which will not result in any degree of unacceptable toxicity, including allergenic and disease states.

The term “binding” refers to a direct association between two molecules, due to, for example, covalent, electrostatic, hydrophobic, and ionic and/or hydrogen-bond interactions, including interactions such as salt bridges and water bridges.

By “coding sequence” is meant any nucleic acid sequence that contributes to the code for the polypeptide product of a gene. By contrast, the term “non-coding sequence” refers to any nucleic acid sequence that does not directly contribute to the code for the polypeptide product of a gene.

Throughout this disclosure, unless the context requires otherwise, the words “comprise,” “comprises,” and “comprising” will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The term “effector function”, or “ADCC effector function” in the context of antibodies refers to the ability of that antibody to engage with other arms of the immune system, including for example, the activation of the classical complement pathway, or through engagement of Fc receptors. Complement dependent pathways are primarily driven by the interaction of C1q with the C1 complex with clustered antibody Fc domains. Antibody dependent cellular cytotoxicity (ADCC), is primarily driven by the interaction of Fc receptors (FcRs) on the surface of effector cells (natural killer cells, macrophages, monocytes and eosinophils) which bind to the Fc region of an IgG which itself is bound to a target cell. Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are at least three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range KD) while FcγRII and FcγRIII are low to intermediate affinity (micromolar range KD). Upon Fc binding a signaling pathway is triggered which results in the secretion of various substances, such as lytic enzymes, perforin, granzymes and tumour necrosis factor, which mediate in the destruction of the target cell. The level of ADCC effector function various for human IgG subtypes. Although this is dependent on the allotype and specific FcvR, in simple terms ADCC effector function is “high” for human IgG1 and IgG3, and “low” for IgG2 and IgG4.

The term “endotoxin free” or “substantially endotoxin free” relates generally to compositions, solvents, and/or vessels that contain at most trace amounts (e.g., amounts having no clinically adverse physiological effects to a subject) of endotoxin, and preferably undetectable amounts of endotoxin. Endotoxins are toxins associated with certain micro-organisms, such as bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art.

Endotoxins can be detected using routine techniques known in the art. For example, the Limulus Amoebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/mg of active compound. Typically, 1 ng lipopolysaccharide (LPS) corresponds to about 1-10 EU.

The term “epitope” includes any determinant, preferably a polypeptide determinant, capable of specific binding to an immunoglobulin or T-cell receptor. An epitope includes a region of an antigen that is bound by an antibody. In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. Epitopes can be contiguous or non-contiguous in relation to the primary structure of the antigen, for example, an HRS polypeptide. In particular embodiments, an epitope comprises, consists, or consists essentially of about, at least about, or no more than about 3, 4, 5, 6, 7, 8, 9, 10, 11, 12, 13, 14, 15, 16, 17, 18, 19, or 20 contiguous amino acids (i.e., a linear epitope) or non-contiguous amino acids (i.e., conformational epitope) of a reference sequence (see, e.g., Table H1) or target molecule described herein.

An “epitope” includes that portion of an antigen or other macromolecule capable of forming a binding interaction that interacts with the variable region binding pocket of a binding protein. Such binding interaction can be manifested as an intermolecular contact with one or more amino acid residues of a CDR. Antigen binding can involve a CDR3 or a CDR3 pair. An epitope can be a linear peptide sequence (i.e., “continuous”) or can be composed of noncontiguous amino acid sequences (i.e., “conformational” or “discontinuous”). A binding protein can recognize one or more amino acid sequences; therefore an epitope can define more than one distinct amino acid sequence. Epitopes recognized by binding protein can be determined by peptide mapping and sequence analysis techniques well known to one of skill in the art. A “cryptic epitope” or a “cryptic binding site” is an epitope or binding site of a protein sequence that is not exposed or substantially protected from recognition within an unmodified polypeptide, but is capable of being recognized by a binding protein of a denatured or proteolyzed polypeptide. Amino acid sequences that are not exposed, or are only partially exposed, in the unmodified polypeptide structure are potential cryptic epitopes. If an epitope is not exposed, or only partially exposed, then it is likely that it is buried within the interior of the polypeptide. Candidate cryptic epitopes can be identified, for example, by examining the three-dimensional structure of an unmodified polypeptide.

The term “half maximal effective concentration” or “EC50” refers to the concentration of an agent (e.g., antibody) as described herein at which it induces a response halfway between the baseline and maximum after some specified exposure time; the EC50 of a graded dose response curve therefore represents the concentration of a compound at which 50% of its maximal effect is observed. EC50 also represents the plasma concentration required for obtaining 50% of a maximum effect in vivo. Similarly, the “EC90” refers to the concentration of an agent or composition at which 90% of its maximal effect is observed. The “EC90” can be calculated from the “EC50” and the Hill slope, or it can be determined from the data directly, using routine knowledge in the art. In some embodiments, the EC50 of an agent (e.g., antibody) is less than about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90, 100, 200 or 500 nM. In some embodiments, an agent will have an EC50 value of about 1 nM or less.

“Immune response” means any immunological response originating from immune system, including responses from the cellular and humeral, innate and adaptive immune systems. Exemplary cellular immune cells include for example, lymphocytes, macrophages, T cells, B cells, NK cells, neutrophils, eosinophils, dendritic cells, mast cells, monocytes, and all subsets thereof. Cellular responses include for example, effector function, cytokine release, phagocytosis, translocation, trafficking, proliferation, differentiation, activation, repression, cell-cell interactions, apoptosis, etc. Humeral responses include for example IgG, IgM, IgA, IgE, responses and their corresponding effector functions.

The “half-life” of an agent such as an antibody can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the amount or concentration of an agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 1.5, 2, 3, 4, 5, 6, 7, 8, 9, 10, 15, 20, 30, 40, 50, 60, 70, 80, 90, 100 or more times (e.g., 500, 1000 times) (including all integers and ranges in between e.g., 1.5, 1.6, 1.7. 1.8, etc.) the amount produced by no composition (e.g., the absence of agent) or a control composition. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease (including all integers and ranges in between) in the amount produced by no composition (e.g., the absence of an agent) or a control composition. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “polynucleotide” and “nucleic acid” includes mRNA, RNA, cRNA, cDNA, and DNA. The term typically refers to polymeric form of nucleotides of at least 10 bases in length, either ribonucleotides or deoxynucleotides or a modified form of either type of nucleotide. The term includes single and double stranded forms of DNA. The terms “isolated DNA” and “isolated polynucleotide” and “isolated nucleic acid” refer to a molecule that has been isolated free of total genomic DNA of a particular species. Therefore, an isolated DNA segment encoding a polypeptide refers to a DNA segment that contains one or more coding sequences yet is substantially isolated away from, or purified free from, total genomic DNA of the species from which the DNA segment is obtained. Also included are non-coding polynucleotides (e.g., primers, probes, oligonucleotides), which do not encode a polypeptide. Also included are recombinant vectors, including, for example, expression vectors, viral vectors, plasmids, cosmids, phagemids, phage, viruses, and the like.

Additional coding or non-coding sequences may, but need not, be present within a polynucleotide described herein, and a polynucleotide may, but need not, be linked to other molecules and/or support materials. Hence, a polynucleotide or expressible polynucleotides, regardless of the length of the coding sequence itself, may be combined with other sequences, for example, expression control sequences.

“Expression control sequences” include regulatory sequences of nucleic acids, or the corresponding amino acids, such as promoters, leaders, enhancers, introns, recognition motifs for RNA, or DNA binding proteins, polyadenylation signals, terminators, internal ribosome entry sites (IRES), secretion signals, subcellular localization signals, and the like, which have the ability to affect the transcription or translation, or subcellular, or cellular location of a coding sequence in a host cell. Exemplary expression control sequences are described in Goeddel; Gene Expression Technology: Methods in Enzymology 185, Academic Press, San Diego, Calif. (1990).

A “promoter” is a DNA regulatory region capable of binding RNA polymerase in a cell and initiating transcription of a downstream (3′ direction) coding sequence. As used herein, the promoter sequence is bounded at its 3′ terminus by the transcription initiation site and extends upstream (5′ direction) to include the minimum number of bases or elements necessary to initiate transcription at levels detectable above background. A transcription initiation site (conveniently defined by mapping with nuclease S1) can be found within a promoter sequence, as well as protein binding domains (consensus sequences) responsible for the binding of RNA polymerase. Eukaryotic promoters can often, but not always, contain “TATA” boxes and “CAT” boxes. Prokaryotic promoters contain Shine-Dalgarno sequences in addition to the −10 and −35 consensus sequences.

A large number of promoters, including constitutive, inducible and repressible promoters, from a variety of different sources are well known in the art. Representative sources include for example, viral, mammalian, insect, plant, yeast, and bacterial cell types), and suitable promoters from these sources are readily available, or can be made synthetically, based on sequences publicly available on line or, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, the RSV promoter. Inducible promoters include the Tet system, (U.S. Pat. Nos. 5,464,758 and 5,814,618), the Ecdysone inducible system (No et al., Proc. Natl. Acad. Sci. (1996) 93 (8): 3346-3351; the T-REx™ system (Invitrogen Carlsbad, Calif.), LacSwitch® (Stratagene, (San Diego, Calif.) and the Cre-ERT tamoxifen inducible recombinase system (Indra et al. Nuc. Acid. Res. (1999) 27 (22): 4324-4327; Nuc. Acid. Res. (2000) 28 (23): e99; U.S. Pat. No. 7,112,715; and Kramer & Fussenegger, Methods Mol. Biol. (2005) 308: 123-144) or any promoter known in the art suitable for expression in the desired cells.

An “expressible polynucleotide” includes a cDNA, RNA, mRNA or other polynucleotide that comprises at least one coding sequence and optionally at least one expression control sequence, for example, a transcriptional and/or translational regulatory element, and which can express an encoded polypeptide upon introduction into a cell, for example, a cell in a subject.

Various viral vectors that can be utilized to deliver an expressible polynucleotide include adenoviral vectors, herpes virus vectors, vaccinia virus vectors, adeno-associated virus (AAV) vectors, and retroviral vectors. In some instances, the retroviral vector is a derivative of a murine or avian retrovirus, or is a lentiviral vector. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), SIV, BIV, HIV and Rous Sarcoma Virus (RSV). A number of additional retroviral vectors can incorporate multiple genes. All of these vectors can transfer or incorporate a gene for a selectable marker so that transduced cells can be identified and generated. By inserting a polypeptide sequence of interest into the viral vector, along with another gene that encodes the ligand for a receptor on a specific target cell, for example, the vector may be made target specific. Retroviral vectors can be made target specific by inserting, for example, a polynucleotide encoding a protein. Illustrative targeting may be accomplished by using an antibody to target the retroviral vector. Those of skill in the art will know of, or can readily ascertain without undue experimentation, specific polynucleotide sequences which can be inserted into the retroviral genome to allow target specific delivery of the retroviral vector.

In particular embodiments, the expressible polynucleotide is a modified RNA or modified mRNA polynucleotide, for example, a non-naturally occurring RNA analog. In certain embodiments, the modified RNA or mRNA polypeptide comprises one or more modified or non-natural bases, for example, a nucleotide base other than adenine (A), guanine (G), cytosine (C), thymine (T), and/or uracil (U). In some embodiments, the modified mRNA comprises one or more modified or non-natural internucleotide linkages. Expressible RNA polynucleotides for delivering an encoded therapeutic polypeptide are described, for example, in Kormann et al., Nat Biotechnol. 29:154-7, 2011; and U.S. Application Nos. 2015/0111248; 2014/0243399; 2014/0147454; and 2013/0245104, which are incorporated by reference in their entireties.

The term “isolated” polypeptide or protein referred to herein means that a subject protein (1) is free of at least some other proteins with which it would typically be found in nature, (2) is essentially free of other proteins from the same source, e.g., from the same species, (3) is expressed by a cell from a different species, (4) has been separated from at least about 50 percent of polynucleotides, lipids, carbohydrates, or other materials with which it is associated in nature, (5) is not associated (by covalent or non-covalent interaction) with portions of a protein with which the “isolated protein” is associated in nature, (6) is operably associated (by covalent or non-covalent interaction) with a polypeptide with which it is not associated in nature, or (7) does not occur in nature. Such an isolated protein can be encoded by genomic DNA, cDNA, mRNA or other RNA, of may be of synthetic origin, or any combination thereof. In certain embodiments, the isolated protein is substantially free from proteins or polypeptides or other contaminants that are found in its natural environment that would interfere with its use (therapeutic, diagnostic, prophylactic, research or otherwise).

In certain embodiments, the “purity” of any given agent (e.g., polypeptide such as an antibody) in a composition may be defined. For instance, certain compositions may comprise an agent such as a polypeptide agent that is at least 80%, 85%, 90%, 91%, 92%, 93%, 94%, 95%, 96%, 97%, 98%, 99%, or 100% pure on a protein basis or a weight-weight basis, including all decimals and ranges in between, as measured, for example and by no means limiting, by high performance liquid chromatography (HPLC), a well-known form of column chromatography used frequently in biochemistry and analytical chemistry to separate, identify, and quantify compounds.

A “lipid nanoparticle” or “solid lipid nanoparticle” refers to one or more spherical nanoparticles with an average diameter of between about 10 to about 1000 nanometers, and which comprise a solid lipid core matrix that can solubilize lipophilic molecules. The lipid core is stabilized by surfactants (e.g., emulsifiers), and can comprise one or more of triglycerides (e.g., tristearin), diglycerides (e.g., glycerol bahenate), monoglycerides (e.g., glycerol monostearate), fatty acids (e.g., stearic acid), steroids (e.g., cholesterol), and waxes (e.g., cetyl palmitate), including combinations thereof. Lipid nanoparticles are described, for example, in Petrilli et al., Curr Pharm Biotechnol. 15:847-55, 2014; and U.S. Pat. Nos. 6,217,912; 6,881,421; 7,402,573; 7,404,969; 7,550,441; 7,727,969; 8,003,621; 8,691,750; 8,871,509; 9,017,726; 9,173,853; 9,220,779; 9,227,917; and 9,278,130, which are incorporated by reference in their entireties. Certain compositions described herein are formulated with one or more lipid nanoparticles.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

Certain embodiments include biologically active “variants” and “fragments” of the polypeptides (e.g., antibodies) described herein, and the polynucleotides that encode the same. “Variants” contain one or more substitutions, additions, deletions, and/or insertions relative to a reference polypeptide or polynucleotide (see, e.g., the Tables and the Sequence Listing). A variant polypeptide or polynucleotide comprises an amino acid or nucleotide sequence with at least about 50%, 55%, 60%, 65%, 70%, 75%, 80%, 85%, 90%, 91%, 92%, 93%, 94%, 95%, 96%, 97%, 98%, 99% or more sequence identity or similarity or homology to a reference sequence, as described herein, and substantially retains the activity of that reference sequence. Also included are sequences that consist of or differ from a reference sequences by the addition, deletion, insertion, or substitution of 1, 2, 3, 4, 5, 6, 7, 8, 9, 10, 11, 12, 13, 14, 15, 16, 17, 18, 19, 20, 30, 40, 50, 60, 70, 80, 90, 100, 110, 120, 130, 140, 150 or more amino acids or nucleotides and which substantially retain the activity of that reference sequence. In certain embodiments, the additions or deletions include C-terminal and/or N-terminal additions and/or deletions.

The terms “sequence identity” or, for example, comprising a “sequence 50% identical to,” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., Nucl. Acids Res. 25:3389, 1997.

The term “solubility” refers to the property of an agent (e.g., antibody) provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 5.0, pH 6.0, pH 7.0, pH 7.4, pH 7.6, pH 7.8, or pH 8.0 (e.g., about pH 5-8). In certain embodiments, solubility is measured in water or a physiological buffer such as PBS or NaCl (with or without NaP). In specific embodiments, solubility is measured at relatively lower pH (e.g., pH 6.0) and relatively higher salt (e.g., 500 mM NaCl and 10 mM NaP). In certain embodiments, solubility is measured in a biological fluid (solvent) such as blood or serum. In certain embodiments, the temperature can be about room temperature (e.g., about 20, 21, 22, 23, 24, 25° C.) or about body temperature (37° C.). In certain embodiments, an agent has a solubility of at least about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 25, 30, 40, 50, 60, 70, 80, 90 or 100 mg/ml at room temperature or at 37° C.

A “subject” or a “subject in need thereof” or a “patient” or a “patient in need thereof” includes a mammalian subject such as a human subject.

“Substantially” or “essentially” means nearly totally or completely, for instance, 95%, 96%, 97%, 98%, 99% or greater of some given quantity.

By “statistically significant,” it is meant that the result was unlikely to have occurred by chance. Statistical significance can be determined by any method known in the art. Commonly used measures of significance include the p-value, which is the frequency or probability with which the observed event would occur, if the null hypothesis were true. If the obtained p-value is smaller than the significance level, then the null hypothesis is rejected. In simple cases, the significance level is defined at a p-value of 0.05 or less.

“Therapeutic response” refers to improvement of symptoms (whether or not sustained) based on administration of one or more therapeutic agents.

As used herein, the terms “therapeutically effective amount”, “therapeutic dose,” “prophylactically effective amount,” or “diagnostically effective amount” is the amount of an agent (e.g., anti-HRS antibody, immunotherapy agent) needed to elicit the desired biological response following administration.

As used herein, “treatment” of a subject (e.g. a mammal, such as a human) or a cell is any type of intervention used in an attempt to alter the natural course of the individual or cell. Treatment includes, but is not limited to, administration of a pharmaceutical composition, and may be performed either prophylactically or subsequent to the initiation of a pathologic event or contact with an etiologic agent. Also included are “prophylactic” treatments, which can be directed to reducing the rate of progression of the disease or condition being treated, delaying the onset of that disease or condition, or reducing the severity of its onset. “Treatment” or “prophylaxis” does not necessarily indicate complete eradication, cure, or prevention of the disease or condition, or associated symptoms thereof.

The term “wild-type” refers to a gene or gene product (e.g., a polypeptide) that is most frequently observed in a population and is thus arbitrarily designed the “normal” or “wild-type” form of the gene.

Each embodiment in this specification is to be applied to every other embodiment unless expressly stated otherwise.

Anti-HRS Antibodies

Certain embodiments include antibodies, and antigen-binding fragments thereof, which specifically bind to a human histidyl-tRNA synthetase polypeptide (“HRS” or “HisRS” or “HARS” polypeptides). Histidyl-tRNA synthetases belong to the class II tRNA synthetase family, which has three highly conserved sequence motifs. Class I and II tRNA synthetases are widely recognized as being responsible for the specific attachment of an amino acid to its cognate tRNA in a two-step reaction: the amino acid (AA) is first activated by ATP to form AA-AMP and then transferred to the acceptor end of the tRNA. The full-length histidyl-tRNA synthetases typically exist either as a cytosolic homodimer, or an alternatively spliced mitochondrial form.

Normally, HRS is thought to be only an intracellular enzyme. Among other aspects of the disclosure described herein, and without being bound by any one particular mode of operation, extracellular or secreted HRS polypeptides may promote avoidance of the immune system by tumor cells and can be specifically targeted by antibodies to “unbrake” the immune system, among other biological processes, resulting in a more productive anti-tumor environment relative to allowing baseline levels of extracellular HRS to exist in patients, which could otherwise decrease the probability of immune attack on the tumor(s).

Certain biological fragments or alternatively spliced isoforms of eukaryotic histidyl-tRNA synthetases, or in some contexts the intact full-length synthetase, modulate certain therapeutically relevant cell-signaling pathways and/or have anti-inflammatory properties. These activities, which are distinct from the classical role of tRNA synthetases in protein synthesis, are referred to herein as “non-canonical activities.” Exemplary splice variants include those disclosed in WO/2010/107825 and WO/2012/021249 and U.S. Pat. Nos. 8,404,242, 8,753,638, and 9,422,539. Specific examples of splice variants include SV9 (HRS(1-60)), SV11 (HRS(1-60)+(399-509)) and SV14(HRS(1-100)+(399-509)).

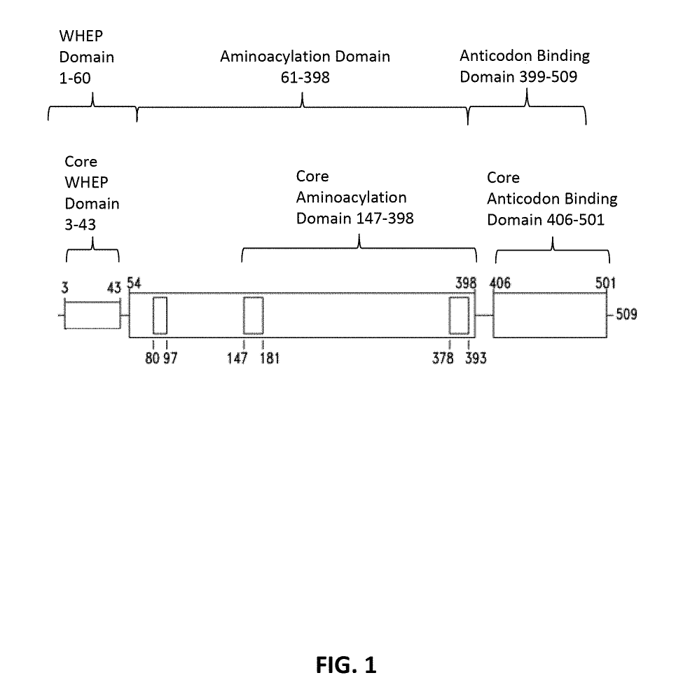

The general structure of human HRS is illustrated in FIG. 1, including the WHEP domain (˜residues 1-60 including a core WHEP domain of ˜residues 3-43), the aminoacylation domain (˜residues 54-398 or ˜core residues 61-398), and the anticodon binding domain (˜residues 399-509 including a core domain of ˜residues 406-501). In some embodiments, an antibody or antigen-binding fragment thereof specifically binds to a full-length human HRS polypeptide, for instance, a human HRS polypeptide that comprises, consists, or consists essentially of residues 1-509 of SEQ ID NO: 1, and/or a variant thereof, for example, a naturally-occurring variant or polymorph (SNP) of full-length human HRS.

Additionally, because the HRS gene can generate a number splice variants, as described herein, a series of common or “universal” epitopes have been identified within the first 60 amino acids of human HRS (the N-terminus), as well as the last 200 amino acids of the C-terminus, which are shared by the majority of the wild type HRS proteins and the majority of the splice variants. In some instances, an anti-HRS polypeptide specifically binds to one or more of such “universal epitopes”. In some instances, the N-terminus potentially provides a greater coverage of possible SVs compared to the C-terminus; however, both approaches can be quite useful depending on the HRS proteins present. In some embodiments, these universal epitopes enable a single antibody or antigen binding fragment thereof to block or clear multiple HRS splice variants or proteolytic fragments thereof, including those having one or more relevant biological activities.

In some embodiments, an antibody or antigen-binding fragment thereof specifically binds to at least one epitope within the N-terminal region of the human HRS polypeptide, for example, within about residues 1-100 of SEQ ID NO: 1, or within about the WHEP domain (˜residues 1-60 or ˜residues 3-43). In certain embodiments, an antibody or antigen-binding fragment thereof specifically binds to at least one epitope within the aminoacylation domain (˜residues 54-398 or ˜residues 61-398) of the human HRS polypeptide. In particular embodiments, an antibody or antigen-binding fragment thereof specifically binds to at least one epitope within the anticodon domain (˜residues 399-509 including a core domain of ˜residues 406-501) of the human HRS polypeptide (SEQ ID NO: 1).

In some embodiments, an antibody or antigen-binding fragment thereof specifically binds to a linear, continuous epitope within the N-terminal domain (˜residues 1-100) optionally within the WHEP domain (˜residues 1-60 or ˜residues 3-43), within the aminoacylation domain (˜residues 54-398 or ˜core residues 61-398), or within the anticodon binding domain (˜residues 399-509 including a core domain of ˜residues 406-501) of the human HRS polypeptide (SEQ ID NO: 1).

In some embodiments, an antibody or antigen-binding fragment thereof specifically binds to a conformational epitope composed of two or more discontinuous epitope regions of the HRS polypeptide. For example, in certain embodiments, an antibody or antigen-binding fragment thereof specifically binds to a conformational epitope comprising a first epitope region within the N-terminal domain optionally within the WHEP domain, and a second epitope region within the aminoacylation domain of the human HRS polypeptide (SEQ ID NO: 1). In some embodiments, an antibody or antigen-binding fragment thereof binds to a conformational epitope comprising a first epitope region within the N-terminal domain optionally within the WHEP domain, and second epitope region within the anticodon binding domain of the human HRS polypeptide (SEQ ID NO: 1). In some embodiments, an antibody or antigen-binding fragment thereof binds to a conformational epitope comprising a first epitope region within the N-terminal domain optionally within the WHEP domain, and second, different epitope region within the N-terminal domain optionally within the WHEP domain of the human HRS polypeptide (SEQ ID NO: 1).

In some embodiments, an antibody or antigen-binding fragment thereof specifically binds to at least one epitope within the N-terminal region, for example, an epitope within about residues 1-100, 10-100, 20-100, 30-100, 40-100, 50-100, 60-100, 70-100, 80-100, 90-100, 1-90, 10-90, 20-90, 30-90, 40-90, 50-90, 60-90, 70-90, 80-90, 1-80, 10-80, 20-80, 30-80, 40-80, 50-80, 60-80, 70-80, 1-70, 10-70, 20-70, 30-70, 40-70, 50-70, 60-70, 1-60, 10-60, 20-60, 30-60, 40-60, 50-60, 1-50, 10-50, 20-50, 30-50, 40-50, 1-40, 10-40, 20-40, 30-40, 1-30, 10-30, 20-30, 1-20, 10-20, or 1-10 of SEQ ID NO:1 (FL human HRS).

In some embodiments, an antibody or antigen-binding fragment thereof specifically binds to at least one epitope within the aminoacylation domain (˜residues 54-398 or ˜residues 61-398), for example, an epitope within about residues 54-398, 54-350, 54-300, 54-250, 54-200, 54-150, 54-100, 61-398, 70-398, 80-398, 90-398, 100-398, 110-398, 120-398, 130-398, 140-398, 150-398, 160-398, 170-398, 180-398, 190-398, 200-398, 210-398, 220-398, 230-398, 240-398, 250-398, 260-398, 270-398, 280-398, 290-398, 300-398, 310-398, 320-398, 330-398, 340-398, 350-398, 360-398, 370-398, 380-398, or 60-388, 60-380, 60-370, 60-360, 60-350, 60-340, 60-330, 60-320, 60-310, 60-300, 60-290, 60-280, 60-270, 60-260, 60-250, 60-240, 60-230, 60-220, 60-210, 60-200, 60-180, 60-170, 60-160, 60-150, 60-140, 60-130, 60-120, 60-110, 60-100, 60-90, 60-80, or 60-70 of SEQ ID NO: 1 (FL human HRS).

In some embodiments, an antibody or antigen-binding fragment thereof specifically binds to at least one epitope in the anticodon binding domain (residues 399-506 or ˜residues 406-501), for example, an epitope within about residues 399-500, 399-490, 399-480, 399-470, 399-460, 399-450, 399-440, 399-430, 399-420, 399-410, or 400-509, 410-509, 420-509, 430-509, 440-509, 450-509, 460-509, 470-509, 480-509, 490-509, or 500-509 of SEQ ID NO: 1 (FL human HRS).

In some embodiments, an antibody or antigen-binding fragment thereof specifically binds to an HRS polypeptide selected from Table H1, or at least one epitope within an HRS polypeptide selected from Table H1.

TABLE H1
Exemplary Human HRS polypeptides

| | | | SEQ ID |
|---|---|---|---|
| Name | Residues | Sequence | NO: |
| FL | 1-509 | MAERAALEELVKLQGERVRGLKQQKASAELIEEEVAKLLKLKA | 1 |
| cytosolic | | QLGPDESKQKFVLKTPKGTRDYSPRQMAVREKVFDVIIRCFKR | |
| wild type | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQPLCIC | |
| HRS(1-500) | 1-500 | MAERAALEELVKLQGERVRGLKQQKASAELIEEEVAKLLKLKA | 2 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKR | |
| HRS(1-501) | 1-501 | MAERAALEELVKLQGERVRGLKQQKASAELIEEEVAKLLKLKA | 3 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRR | |
| HRS(1-502) | 1-502 | MAERAALEELVKLQGERVRGLKQQKASAELIEEEVAKLLKLKA | 4 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRT | |
| HRS(1-503) | 1-503 | MAERAALEELVKLQGERVRGLKQQKASAELIEEEVAKLLKLKA | 5 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTG | |
| HRS(1-504) | 1-504 | MAERAALEELVKLQGERVRGLKQQKASAELIEEEVAKLLKLKA | 6 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQ | |
| HRS(1-505) | 1-505 | MAERAALEELVKLQGERVRGLKQQKASAELIEEEVAKLLKLKA | 7 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQP | |
| HisRS1^{N8} | 1-506 | MAERAALEELVKLQGERVRGLKQQKASAELIEEEVAKLLKLKA | 8 |
| HRS(1-506) | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQPL | |
| HRS(2-506) | 2-506 | AERAALEELVKLQGERVRGLKQQKASAELIEEEVAKLLKLKAQ | 9 |
| | | LGPDESKQKFVLKTPKGTRDYSPRQMAVREKVFDVIIRCFKRH | |
| | | GAEVIDTPVFELKETLMGKYGEDSKLIYDLKDQGGELLSLRYD | |
| | | LTVPFARYLAMNKLTNIKRYHIAKVYRRDNPAMTRGRYREFYQ | |
| | | CDFDIAGNFDPMIPDAECLKIMCEILSSLQIGDFLVKVNDRRI | |
| | | LDGMFAICGVSDSKFRTICSSVDKLDKVSWEEVKNEMVGEKGL | |
| | | APEVADRIGDYVQQHGGVSLVEQLLQDPKLSQNKQALEGLGDL | |
| | | KLLFEYLTLFGIDDKISFDLSLARGLDYYTGVIYEAVLLQTPA | |
| | | QAGEEPLGVGSVAAGGRYDGLVGMFDPKGRKVPCVGLSIGVER | |
| | | IFSIVEQRLEALEEKIRTTETQVLVASAQKKLLEERLKLVSEL | |
| | | WDAGIKAELLYKKNPKLLNQLQYCEEAGIPLVAIIGEQELKDG | |
| | | VIKLRSVTSREEVDVRREDLVEEIKRRTGQPL | |
| HRS(1-507) | 1-507 | MAERAALEELVKLQGERVRGLKQQKASAELIEEEVAKLLKLKA | 10 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQPLC | |
| HRS(1-508) | 1-508 | MAERAALEELVKLQGERVRGLKQQKASAELIEEEVAKLLKLKA | 11 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTETQVLVASAQKKLLEERLKLVSE | |
| | | LWDAGIKAELLYKKNPKLLNQLQYCEEAGIPLVAIIGEQELKD | |
| | | GVIKLRSVTSREEVDVRREDLVEEIKRRTGQPLCI | |
| HisRS1^{N6} | 1-48 | MAERAALEELVKLQGERVRGLKQQKASAELIEEEVAKLLK | 422 |
| HRS(1-48) | | LKAQLGPD | |
| | 1-80 | MAERAALEELVKLQGERVRGLKQQKASAELIEEEVAKLLKLKA | 423 |
| | | QLGPDESKQKFVLKTPKGTRDYSPRQMAVREKVFDVI | |
| | 1-79 | MAERAALEELVKLQGERVRGLKQQKASAELIEEEVAKLLK | 424 |
| | | LKAQLGPDESKQKFVLKTPKGTRDYSPRQMAVREKVFDV | |
| | 1-78 | MAERAALEELVKLQGERVRGLKQQKASAELIEEEVAKLLKLKA | 425 |
| | | QLGPDESKQKFVLKTPKGTRDYSPRQMAVREKVFD | |
| | 1-77 | MAERAALEELVKLQGERVRGLKQQKASAELIEEEVAKLLKLKA | 426 |
| | | QLGPDESKQKFVLKTPKGTRDYSPRQMAVREKVF | |
| | 1-76 | MAERAALEELVKLQGERVRGLKQQKASAELIEEEVAKLLKLKA | 427 |
| | | QLGPDESKQKFVLKTPKGTRDYSPRQMAVREKV | |
| | 1-75 | MAERAALEELVKLQGERVRGLKQQKASAELIEEEVAKLLKLKA | 428 |
| | | QLGPDESKQKFVLKTPKGTRDYSPRQMAVREK | |
| | 1-74 | MAERAALEELVKLQGERVRGLKQQKASAELIEEEVAKLLKLKA | 429 |
| | | QLGPDESKQKFVLKTPKGTRDYSPRQMAVRE | |
| | 1-73 | MAERAALEELVKLQGERVRGLKQQKASAELIEEEVAKLLKLKA | 430 |
| | | QLGPDESKQKFVLKTPKGTRDYSPRQMAVR | |
| | 1-72 | MAERAALEELVKLQGERVRGLKQQKASAELIEEEVAKLLKLKA | 431 |
| | | QLGPDESKQKFVLKTPKGTRDYSPRQMAV | |
| | 1-71 | MAERAALEELVKLQGERVRGLKQQKASAELIEEEVAKLLKLKA | 432 |
| | | QLGPDESKQKFVLKTPKGTRDYSPRQMA | |
| | 1-70 | MAERAALEELVKLQGERVRGLKQQKASAELIEEEVAKLLKLKA | 433 |
| | | QLGPDESKQKFVLKTPKGTRDYSPRQM | |
| | 1-69 | MAERAALEELVKLQGERVRGLKQQKASAELIEEEVAKLLKLKA | 434 |
| | | QLGPDESKQKFVLKTPKGTRDYSPRQ | |
| | 1-68 | MAERAALEELVKLQGERVRGLKQQKASAELIEEEVAKLLKLKA | 435 |
| | | QLGPDESKQKFVLKTPKGTRDYSPR | |
| | 1-67 | MAERAALEELVKLQGERVRGLKQQKASAELIEEEVAKLLKLKA | 436 |
| | | QLGPDESKQKFVLKTPKGTRDYSP | |
| | 1-66 | MAERAALEELVKLQGERVRGLKQQKASAELIEEEVAKLLKLKA | 437 |
| | | QLGPDESKQKFVLKTPKGTRDYS | |
| | 1-65 | MAERAALEELVKLQGERVRGLKQQKASAELIEEEVAKLLKLKA | 438 |
| | | QLGPDESKQKFVLKTPKGTRDY | |
| | 1-64 | MAERAALEELVKLQGERVRGLKQQKASAELIEEEVAKLLKLKA | 439 |
| | | QLGPDESKQKFVLKTPKGTRD | |
| | 1-63 | MAERAALEELVKLQGERVRGLKQQKASAELIEEEVAKLLKLKA | 440 |
| | | QLGPDESKQKFVLKTPKGTR | |
| | 1-62 | MAERAALEELVKLQGERVRGLKQQKASAELIEEEVAKLLKLKA | 441 |
| | | QLGPDESKQKFVLKTPKGT | |
| | 1-61 | MAERAALEELVKLQGERVRGLKQQKASAELIEEEVAKLLKLKA | 442 |
| | | QLGPDESKQKFVLKTPKG | |
| | 1-60 | MAERAALEELVKLQGERVRGLKQQKASAELIEEEVAKLLKLKA | 443 |
| | | QLGPDESKQKFVLKTPK | |
| | 1-59 | MAERAALEELVKLQGERVRGLKQQKASAELIEEEVAKLLKLKA | 444 |
| | | QLGPDESKQKFVLKTP | |
| | 1-58 | MAERAALEELVKLQGERVRGLKQQKASAELIEEEVAKLLKLKA | 445 |
| | | QLGPDESKQKFVLKT | |
| | 1-57 | MAERAALEELVKLQGERVRGLKQQKASAELIEEEVAKLLKLKA | 446 |
| | | QLGPDESKQKFVLK | |
| | 1-56 | MAERAALEELVKLQGERVRGLKQQKASAELIEEEVAKLLKLKA | 447 |
| | | QLGPDESKQKFVL | |
| | 1-55 | MAERAALEELVKLQGERVRGLKQQKASAELIEEEVAKLLKLKA | 448 |
| | | QLGPDESKQKFV | |
| | 1-54 | MAERAALEELVKLQGERVRGLKQQKASAELIEEEVAKLLKLKA | 449 |
| | | QLGPDESKQKF | |
| | 1-53 | MAERAALEELVKLQGERVRGLKQQKASAELIEEEVAKLLKLKA | 450 |
| | | QLGPDESKQK | |
| | 1-52 | MAERAALEELVKLQGERVRGLKQQKASAELIEEEVAKLLKLKA | 451 |
| | | QLGPDESKQ | |
| | 1-51 | MAERAALEELVKLQGERVRGLKQQKASAELIEEEVAKLLKLKA | 452 |
| | | QLGPDESK | |
| | 1-50 | MAERAALEELVKLQGERVRGLKQQKASAELIEEEVAKLLKLKA | 453 |
| | | QLGPDES | |
| | 1-49 | MAERAALEELVKLQGERVRGLKQQKASAELIEEEVAKLLKLKA | 454 |
| | | QLGPDE | |
| | 1-48 | MAERAALEELVKLQGERVRGLKQQKASAELIEEEVAKLLKLKA | 455 |
| | | QLGPD | |
| | 1-47 | MAERAALEELVKLQGERVRGLKQQKASAELIEEEVAKLLKLKA | 456 |
| | | QLGP | |
| | 1-46 | MAERAALEELVKLQGERVRGLKQQKASAELIEEEVAKLLKLKA | 457 |
| | | QLG | |
| | 1-45 | MAERAALEELVKLQGERVRGLKQQKASAELIEEEVAKLLKLKA | 458 |
| | | QL | |
| | 1-44 | MAERAALEELVKLQGERVRGLKQQKASAELIEEEVAKLLKLKA | 459 |
| | | Q | |
| | 1-43 | MAERAALEELVKLQGERVRGLKQQKASAELIEEEVAKLLK | 460 |
| | | LKA | |
| | 1-42 | MAERAALEELVKLQGERVRGLKQQKASAELIEEEVAKLLK | 461 |
| | | LK | |
| | 1-41 | MAERAALEELVKLQGERVRGLKQQKASAELIEEEVAKLLK | 462 |
| | | L | |
| | 1-40 | MAERAALEELVKLQGERVRGLKQQKASAELIEEEVAKLLK | 463 |
| | 1-39 | MAERAALEELVKLQGERVRGLKQQKASAELIEEEVAKLL | 464 |
| | 1-38 | MAERAALEELVKLQGERVRGLKQQKASAELIEEEVAKL | 465 |
| | 1-37 | MAERAALEELVKLQGERVRGLKQQKASAELIEEEVAK | 466 |
| | 1-36 | MAERAALEELVKLQGERVRGLKQQKASAELIEEEVA | 467 |
| | 1-35 | MAERAALEELVKLQGERVRGLKQQKASAELIEEEV | 468 |
| | 1-34 | MAERAALEELVKLQGERVRGLKQQKASAELIEEE | 469 |
| | 1-33 | MAERAALEELVKLQGERVRGLKQQKASAELIEE | 470 |
| | 1-32 | MAERAALEELVKLQGERVRGLKQQKASAELIE | 471 |
| | 1-31 | MAERAALEELVKLQGERVRGLKQQKASAELI | 472 |
| | 1-30 | MAERAALEELVKLQGERVRGLKQQKASAEL | 473 |
| | 2-80 | AERAALEELVKLQGERVRGLKQQKASAELIEEEVAKLLKLKAQ | 474 |
| | | LGPDESKQKFVLKTPKGTRDYSPRQMAVREKVFDVI | |
| | 3-80 | ERAALEELVKLQGERVRGLKQQKASAELIEEEVAKLLKLKAQL | 475 |
| | | GPDESKQKFVLKTPKGTRDYSPRQMAVREKVFDVI | |
| | 4-80 | RAALEELVKLQGERVRGLKQQKASAELIEEEVAKLLKLKAQLG | 476 |
| | | PDESKQKFVLKTPKGTRDYSPRQMAVREKVFDVI | |
| | 5-80 | AALEELVKLQGERVRGLKQQKASAELIEEEVAKLLKLKAQLGP | 477 |
| | | DESKQKFVLKTPKGTRDYSPRQMAVREKVFDVI | |
| | 6-80 | ALEELVKLQGERVRGLKQQKASAELIEEEVAKLLKLKAQLGPD | 478 |
| | | ESKQKFVLKTPKGTRDYSPRQMAVREKVFDVI | |
| | 7-80 | LEELVKLQGERVRGLKQQKASAELIEEEVAKLLKLKAQLGPDE | 479 |
| | | SKQKFVLKTPKGTRDYSPRQMAVREKVFDVI | |
| | 8-80 | EELVKLQGERVRGLKQQKASAELIEEEVAKLLKLKAQLGPDES | 480 |
| | | KQKFVLKTPKGTRDYSPRQMAVREKVFDVI | |
| | 9-80 | ELVKLQGERVRGLKQQKASAELIEEEVAKLLKLKAQLGPDESK | 481 |
| | | QKFVLKTPKGTRDYSPRQMAVREKVFDVI | |
| | 10-80 | LVKLQGERVRGLKQQKASAELIEEEVAKLLKLKAQLGPDESKQ | 482 |
| | | KFVLKTPKGTRDYSPRQMAVREKVFDVI | |
| | 11-80 | VKLQGERVRGLKQQKASAELIEEEVAKLLKLKAQLGPDESKQK | 483 |
| | | FVLKTPKGTRDYSPRQMAVREKVFDVI | |
| | 12-80 | KLQGERVRGLKQQKASAELIEEEVAKLLKLKAQLGPDESKQKF | 484 |
| | | VLKTPKGTRDYSPRQMAVREKVFDVI | |
| | 13-80 | LQGERVRGLKQQKASAELIEEEVAKLLKLKAQLGPDESKQKFV | 485 |
| | | LKTPKGTRDYSPRQMAVREKVFDVI | |
| | 14-80 | QGERVRGLKQQKASAELIEEEVAKLLKLKAQLGPDESKQKFVL | 486 |
| | | KTPKGTRDYSPRQMAVREKVFDVI | |
| | 15-80 | GERVRGLKQQKASAELIEEEVAKLLKLKAQLGPDESKQKFVLK | 487 |
| | | TPKGTRDYSPRQMAVREKVFDVI | |
| | 16-80 | RVRGLKQQKASAELIEEEVAKLLKLKAQLGPDESKQKFVLKTP | 488 |
| | | KGTRDYSPRQMAVREKVFDVI | |
| | 17-80 | VRGLKQQKASAELIEEEVAKLLKLKAQLGPDESKQKFVLKTPK | 489 |
| | | GTRDYSPRQMAVREKVFDVI | |
| | 18-80 | RGLKQQKASAELIEEEVAKLLKLKAQLGPDESKQKFVLKTPKG | 490 |
| | | TRDYSPRQMAVREKVFDVI | |
| | 19-80 | GLKQQKASAELIEEEVAKLLKLKAQLGPDESKQKFVLKTPKGT | 491 |
| | | RDYSPRQMAVREKVFDVI | |
| | 20-80 | LKQQKASAELIEEEVAKLLKLKAQLGPDESKQKFVLKTPKGTR | 492 |
| | | DYSPRQMAVREKVFDVI | |
| | 21-80 | KQQKASAELIEEEVAKLLKLKAQLGPDESKQKFVLKTPKGTRD | 493 |
| | | YSPRQMAVREKVFDVI | |
| | 22-80 | QQKASAELIEEEVAKLLKLKAQLGPDESKQKFVLKTPKGTRDY | 494 |
| | | SPRQMAVREKVFDVI | |
| | 23-80 | QKASAELIEEEVAKLLKLKAQLGPDESKQKFVLKTPKGTRDYS | 495 |
| | | PRQMAVREKVFDVI | |
| | 24-80 | KASAELIEEEVAKLLKLKAQLGPDESKQKFVLKTPKGTRDYSP | 496 |
| | | RQMAVREKVFDVI | |
| | 25-80 | ASAELIEEEVAKLLKLKAQLGPDESKQKFVLKTPKGTRDYSPR | 497 |
| | | QMAVREKVFDVI | |
| | 26-80 | SAELIEEEVAKLLKLKAQLGPDESKQKFVLKTPKGTRDYSPRQ | 498 |
| | | MAVREKVFDVI | |
| | 27-80 | AELIEEEVAKLLKLKAQLGPDESKQKFVLKTPKGTRDYSPRQM | 499 |
| | | AVREKVFDVI | |
| | 28-80 | ELIEEEVAKLLKLKAQLGPDESKQKFVLKTPKGTRDYSPRQMA | 500 |
| | | VREKVFDVI | |
| | 29-80 | LIEEEVAKLLKLKAQLGPDESKQKFVLKTPKGTRDYSPRQMAV | 501 |
| | | REKVFDVI | |
| | 30-80 | IEEEVAKLLKLKAQLGPDESKQKFVLKTPKGTRDYSPRQMAVR | 502 |
| | | EKVFDVI | |
| | 31-80 | EEEVAKLLKLKAQLGPDESKQKFVLKTPKGTRDYSPRQMAVRE | 503 |
| | | KVFDVI | |
| | 32-80 | EEVAKLLKLKAQLGPDESKQKFVLKTPKGTRDYSPRQMAVREK | 504 |
| | | VFDVI | |
| | 33-80 | EVAKLLKLKAQLGPDESKQKFVLKTPKGTRDYSPRQMAVREKV | 505 |
| | | FDVI | |
| | 34-80 | VAKLLKLKAQLGPDESKQKFVLKTPKGTRDYSPRQMAVREKVF | 506 |
| | | DVI | |
| | 35-80 | AKLLKLKAQLGPDESKQKFVLKTPKGTRDYSPRQMAVREKVFD | 507 |
| | | VI | |
| | 36-80 | KLLKLKAQLGPDESKQKFVLKTPKGTRDYSPRQMAVREKVFDV | 508 |
| | | I | |
| | 37-80 | LLKLKAQLGPDESKQKFVLKTPKGTRDYSPRQMAVREKVFDVI | 509 |
| | 38-80 | LKLKAQLGPDESKQKFVLKTPKGTRDYSPRQMAVREKVFDVI | 510 |
| | 39-80 | KLKAQLGPDESKQKFVLKTPKGTRDYSPRQMAVREKVFDVI | 511 |
| | 40-80 | LKAQLGPDESKQKFVLKTPKGTRDYSPRQMAVREKVFDVI | 512 |
| | 10-60 | LVKLQGERVRGLKQQKASAELIEEEVAKLLKLKAQLGPDESKQ | 513 |
| | | KFVLKTPK | |
| | 11-60 | VKLQGERVRGLKQQKASAELIEEEVAKLLKLKAQLGPDESKQK | 514 |
| | | FVLKTPK | |
| | 12-60 | KLQGERVRGLKQQKASAELIEEEVAKLLKLKAQLGPDESKQKF | 515 |
| | | VLKTPK | |
| | 13-60 | LQGERVRGLKQQKASAELIEEEVAKLLKLKAQLGPDESKQKFV | 516 |
| | | LKTPK | |
| | 14-60 | QGERVRGLKQQKASAELIEEEVAKLLKLKAQLGPDESKQKFVL | 517 |
| | | KTPK | |
| | 15-60 | GERVRGLKQQKASAELIEEEVAKLLKLKAQLGPDESKQKFVLK | 518 |
| | | TPK | |
| | 16-60 | ERVRGLKQQKASAELIEEEVAKLLKLKAQLGPDESKQKFVLKT | 519 |
| | | PK | |
| | 17-60 | RVRGLKQQKASAELIEEEVAKLLKLKAQLGPDESKQKFVLKTP | 520 |
| | | K | |
| | 18-60 | VRGLKQQKASAELIEEEVAKLLKLKAQLGPDESKQKFVLKTPK | 521 |
| | 19-60 | RGLKQQKASAELIEEEVAKLLKLKAQLGPDESKQKFVLKTPK | 522 |
| | 20-60 | GLKQQKASAELIEEEVAKLLKLKAQLGPDESKQKFVLKTPK | 523 |
| | 21-60 | LKQQKASAELIEEEVAKLLKLKAQLGPDESKQKFVLKTPK | 524 |
| | 22-60 | KQQKASAELIEEEVAKLLKLKAQLGPDESKQKFVLKTPK | 525 |
| | 23-60 | QQKASAELIEEEVAKLLKLKAQLGPDESKQKFVLKTPK | 526 |
| | 24-60 | QKASAELIEEEVAKLLKLKAQLGPDESKQKFVLKTPK | 527 |
| | 25-60 | KASAELIEEEVAKLLKLKAQLGPDESKQKFVLKTPK | 528 |
| | 26-60 | ASAELIEEEVAKLLKLKAQLGPDESKQKFVLKTPK | 529 |
| | 27-60 | SAELIEEEVAKLLKLKAQLGPDESKQKFVLKTPK | 530 |
| | 28-60 | AELIEEEVAKLLKLKAQLGPDESKQKFVLKTPK | 531 |
| | 29-60 | ELIEEEVAKLLKLKAQLGPDESKQKFVLKTPK | 532 |
| | 30-60 | LIEEEVAKLLKLKAQLGPDESKQKFVLKTPK | 533 |
| | 31-60 | IEEEVAKLLKLKAQLGPDESKQKFVLKTPK | 534 |
| | 32-60 | EEEVAKLLKLKAQLGPDESKQKFVLKTPK | 535 |
| | 33-60 | EEVAKLLKLKAQLGPDESKQKFVLKTPK | 536 |
| | 34-60 | EVAKLLKLKAQLGPDESKQKFVLKTPK | 537 |
| | 35-60 | VAKLLKLKAQLGPDESKQKFVLKTPK | 538 |
| | 36-60 | AKLLKLKAQLGPDESKQKFVLKTPK | 539 |
| | 37-60 | KLLKLKAQLGPDESKQKFVLKTPK | 540 |
| | 38-60 | LLKLKAQLGPDESKQKFVLKTPK | 541 |
| | 39-60 | LKLKAQLGPDESKQKFVLKTPK | 542 |
| | 40-60 | KLKAQLGPDESKQKFVLKTPK | 543 |
| | 10-50 | LVKLQGERVRGLKQQKASAELIEEEVAKLLKLKAQLGPDES | 544 |
| | 11-50 | VKLQGERVRGLKQQKASAELIEEEVAKLLKLKAQLGPDES | 545 |
| | 12-50 | KLQGERVRGLKQQKASAELIEEEVAKLLKLKAQLGPDES | 546 |
| | 13-50 | LQGERVRGLKQQKASAELIEEEVAKLLKLKAQLGPDES | 547 |
| | 14-50 | QGERVRGLKQQKASAELIEEEVAKLLKLKAQLGPDES | 548 |
| | 15-50 | GERVRGLKQQKASAELIEEEVAKLLKLKAQLGPDES | 549 |
| | 16-50 | ERVRGLKQQKASAELIEEEVAKLLKLKAQLGPDES | 550 |
| | 17-50 | RVRGLKQQKASAELIEEEVAKLLKLKAQLGPDES | 551 |
| | 18-50 | VRGLKQQKASAELIEEEVAKLLKLKAQLGPDES | 552 |
| | 19-50 | RGLKQQKASAELIEEEVAKLLKLKAQLGPDES | 553 |
| | 20-50 | GLKQQKASAELIEEEVAKLLKLKAQLGPDES | 554 |
| | 21-50 | LKQQKASAELIEEEVAKLLKLKAQLGPDES | 555 |
| | 22-50 | KQQKASAELIEEEVAKLLKLKAQLGPDES | 556 |
| | 23-50 | QQKASAELIEEEVAKLLKLKAQLGPDES | 557 |
| | 24-50 | QKASAELIEEEVAKLLKLKAQLGPDES | 558 |
| | 25-50 | KASAELIEEEVAKLLKLKAQLGPDES | 559 |
| | 26-50 | ASAELIEEEVAKLLKLKAQLGPDES | 560 |
| | 27-50 | SAELIEEEVAKLLKLKAQLGPDES | 561 |
| | 28-50 | AELIEEEVAKLLKLKAQLGPDES | 562 |
| | 29-50 | ELIEEEVAKLLKLKAQLGPDES | 563 |
| | 30-50 | LIEEEVAKLLKLKAQLGPDES | 564 |
| | 31-50 | IEEEVAKLLKLKAQLGPDES | 565 |
| | 32-50 | EEEVAKLLKLKAQLGPDES | 566 |
| | 33-50 | EEVAKLLKLKAQLGPDES | 567 |
| | 34-50 | EVAKLLKLKAQLGPDES | 568 |
| | 35-50 | VAKLLKLKAQLGPDES | 569 |
| | 36-50 | AKLLKLKAQLGPDES | 570 |
| | 37-50 | KLLKLKAQLGPDES | 571 |
| | 38-50 | LLKLKAQLGPDES | 572 |
| | 39-50 | LKLKAQLGPDES | 573 |
| | 40-50 | KLKAQLGPDES | 574 |
| | 10-40 | LVKLQGERVRGLKQQKASAELIEEEVAKLLK | 575 |
| | 11-40 | VKLQGERVRGLKQQKASAELIEEEVAKLLK | 576 |
| | 12-40 | KLQGERVRGLKQQKASAELIEEEVAKLLK | 577 |
| | 13-40 | LQGERVRGLKQQKASAELIEEEVAKLLK | 578 |
| | 14-40 | QGERVRGLKQQKASAELIEEEVAKLLK | 579 |
| | 15-40 | GERVRGLKQQKASAELIEEEVAKLLK | 580 |
| | 16-40 | ERVRGLKQQKASAELIEEEVAKLLK | 581 |
| | 17-40 | RVRGLKQQKASAELIEEEVAKLLK | 582 |
| | 18-40 | VRGLKQQKASAELIEEEVAKLLK | 583 |
| | 19-40 | RGLKQQKASAELIEEEVAKLLK | 584 |
| | 20-40 | GLKQQKASAELIEEEVAKLLK | 585 |
| | 21-40 | LKQQKASAELIEEEVAKLLK | 586 |
| | 22-40 | KQQKASAELIEEEVAKLLK | 587 |
| | 23-40 | QQKASAELIEEEVAKLLK | 588 |
| | 24-40 | QKASAELIEEEVAKLLK | 589 |
| | 25-40 | KASAELIEEEVAKLLK | 590 |
| | 26-40 | ASAELIEEEVAKLLK | 591 |
| | 27-40 | SAELIEEEVAKLLK | 592 |
| | 28-40 | AELIEEEVAKLLK | 593 |
| | 29-40 | ELIEEEVAKLLK | 594 |
| | 30-40 | LIEEEVAKLLK | 595 |
| HisRS1^{N1} | 1-141 | MAERAALEELVKLQGERVRGLKQQKASAELIEEEVAKLLKLKA | 596 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAM | |
| HisRS1^{N2} | 1-408 | MAERAALEELVKLQGERVRGLKQQKASAELIEEEVAKLLKLKA | 597 |
| | | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVSWEEVKNEMVGEKG | |
| | | LAPEVADRIGDYVQQHGGVSLVEQLLQDPKLSQNKQALEGLGD | |
| | | LKLLFEYLTLFGIDDKISFDLSLARGLDYYTGVIYEAVLLQTP | |
| | | AQAGEEPLGVGSVAAGGRYDGLVGMFDPKGRKVPCVGLSIGVE | |
| | | RIFSIVEQRLEALEEKIRTTE | |
| HisRS1^{N3} | 1-113 | MAERAALEELVKLQGERVRGLKQQKASAELIEEEVAKLLKLKA | 598 |
| | | QLGPDESKQKFVLKTPKGTRDYSPRQMAVREKVFDVIIRCFKR | |
| | | HGAEVIDTPVFELKETLMGKYGEDSKL | |
| HisRS1^{N4} | 1-60 | MAERAALEELVKLQGERVRGLKQQKASAELIEEEVAKLLKLKA | 599 |
| | | QLGPDESKQKFVLKTPK | |
| HisRS1^{N5} | 1-243 + | MAERAALEELVKLQGERVRGLKQQKASAELIEEEVAKLLKLKA | 600 |
| | 27aa | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCDFDIAGNFDPMIPDAECLKIMCEILSSLQIGDFLVKVNDRR | |
| | | ILDGMFAICGVSDSKFRTICSSVDKLDKVGYPWWNSCSRILNY | |
| | | PKTSRPWRAWET | |
| HisRS1^{c1} | 405-509 | RTTETQVLVASAQKKLLEERLKLVSELWDAGIKAELLYKKNPK | 601 |
| | | LLNQLQYCEEAGIPLVAIIGEQELKDGVIKLRSVTSREEVDVR | |
| | | REDLVEEIKRRTGQPLCIC | |
| HisRS1^{c2} | 1-60 + | MAERAALEELVKLQGERVRGLKQQKASAELIEEEVAKLLKLKA | 602 |
| | 175-509 | QLGPDESKQKFVLKTPKDFDIAGNFDPMIPDAECLKIMCEILS | |
| | | SLQIGDFLVKVNDRRILDGMFAICGVSDSKFRTICSSVDKLDK | |
| | | VSWEEVKNEMVGEKGLAPEVADRIGDYVQQHGGVSLVEQLLQD | |
| | | PKLSQNKQALEGLGDLKLLFEYLTLFGIDDKISFDLSLARGLD | |
| | | YYTGVIYEAVLLQTPAQAGEEPLGVGSVAAGGRYDGLVGMFDP | |
| | | KGRKVPCVGLSIGVERIFSIVEQRLEALEEKIRTTETQVLVAS | |
| | | AQKKLLEERLKLVSELWDAGIKAELLYKKNPKLLNQLQYCEEA | |
| | | GIPLVAIIGEQELKDGVIKLRSVTSREEVDVRREDLVEEIKRR | |
| | | TGQPLCIC | |
| HisRS1^{c3} | 1-60 + | MAERAALEELVKLQGERVRGLKQQKASAELIEEEVAKLLKLKA | 603 |
| | 211-509 | QLGPDESKQKFVLKTPKVNDRRILDGMFAICGVSDSKFRTICS | |
| | | SVDKLDKVSWEEVKNEMVGEKGLAPEVADRIGDYVQQHGGVSL | |
| | | VEQLLQDPKLSQNKQALEGLGDLKLLFEYLTLFGIDDKISFDL | |
| | | SLARGLDYYTGVIYEAVLLQTPAQAGEEPLGVGSVAAGGRYDG | |
| | | LVGMFDPKGRKVPCVGLSIGVERIFSIVEQRLEALEEKIRTTE | |
| | | TQVLVASAQKKLLEERLKLVSELWDAGIKAELLYKKNPKLLNQ | |
| | | LQYCEEAGIPLVAIIGEQELKDGVIKLRSVTSREEVDVRREDL | |
| | | VEEIKRRTGQPLCIC | |
| HisRS1^{c4} | 1-100 + | MAERAALEELVKLQGERVRGLKQQKASAELIEEEVAKLLKLKA | 604 |
| | 211-509 | QLGPDESKQKFVLKTPKGTRDYSPRQMAVREKVFDVIIRCFKR | |
| | | HGAEVIDTPVFELKVNDRRILDGMFAICGVSDSKFRTICSSVD | |
| | | KLDKVSWEEVKNEMVGEKGLAPEVADRIGDYVQQHGGVSLVEQ | |
| | | LLQDPKLSQNKQALEGLGDLKLLFEYLTLFGIDDKISFDLSLA | |
| | | RGLDYYTGVIYEAVLLQTPAQAGEEPLGVGSVAAGGRYDGLVG | |
| | | MFDPKGRKVPCVGLSIGVERIFSIVEQRLEALEEKIRTTETQV | |
| | | LVASAQKKLLEERLKLVSELWDAGIKAELLYKKNPKLLNQLQY | |
| | | CEEAGIPLVAIIGEQELKDGVIKLRSVTSREEVDVRREDLVEE | |
| | | IKRRTGQPLCIC | |
| HisRS1^{c5} | 1-174 + | MAERAALEELVKLQGERVRGLKQQKASAELIEEEVAKLLKLKA | 605 |
| | 211-509 | QLGPDESKQKFVLKTPKGTRDYSPRQMAVREKVFDVIIRCFKR | |
| | | HGAEVIDTPVFELKETLMGKYGEDSKLIYDLKDQGGELLSLRY | |
| | | DLTVPFARYLAMNKLTNIKRYHIAKVYRRDNPAMTRGRYREFY | |
| | | QCVNDRRILDGMFAICGVSDSKFRTICSSVDKLDKVSWEEVKN | |
| | | EMVGEKGLAPEVADRIGDYVQQHGGVSLVEQLLQDPKLSQNKQ | |
| | | ALEGLGDLKLLFEYLTLFGIDDKISFDLSLARGLDYYTGVIYE | |
| | | AVLLQTPAQAGEEPLGVGSVAAGGRYDGLVGMFDPKGRKVPCV | |
| | | GLSIGVERIFSIVEQRLEALEEKIRTTETQVLVASAQKKLLEE | |
| | | RLKLVSELWDAGIKAELLYKKNPKLLNQLQYCEEAGIPLVAII | |
| | | GEQELKDGVIKLRSVTSREEVDVRREDLVEEIKRRTGQPLCIC | |
| HisRS1^{c6} | 1-60 + | MAERAALEELVKLQGERVRGLKQQKASAELIEEEVAKLLKLKA | 606 |
| | 101-509 | QLGPDESKQKFVLKTPKETLMGKYGEDSKLIYDLKDQGGELLS | |
| | | LRYDLTVPFARYLAMNKLTNIKRYHIAKVYRRDNPAMTRGRYR | |
| | | EFYQCDFDIAGNFDPMIPDAECLKIMCEILSSLQIGDFLVKVN | |
| | | DRRILDGMFAICGVSDSKFRTICSSVDKLDKVSWEEVKNEMVG | |
| | | EKGLAPEVADRIGDYVQQHGGVSLVEQLLQDPKLSQNKQALEG | |
| | | LGDLKLLFEYLTLFGIDDKISFDLSLARGLDYYTGVIYEAVLL | |
| | | QTPAQAGEEPLGVGSVAAGGRYDGLVGMFDPKGRKVPCVGLSI | |
| | | GVERIFSIVEQRLEALEEKIRTTETQVLVASAQKKLLEERLKL | |
| | | VSELWDAGIKAELLYKKNPKLLNQLQYCEEAGIPLVAIIGEQE | |
| | | LKDGVIKLRSVTSREEVDVRREDLVEEIKRRTGQPLCIC | |
| HisRS1^{c7} | P1-100 + | MAERAALEELVKLQGERVRGLKQQKASAELIEEEVAKLLKLKA | 607 |
| | 175-509 | QLGPDESKQKFVLKTPKGTRDYSPRQMAVREKVFDVIIRCFKR | |
| | | HGAEVIDTPVFELKDFDIAGNFDPMIPDAECLKIMCEILSSLQ | |
| | | IGDFLVKVNDRRILDGMFAICGVSDSKFRTICSSVDKLDKVSW | |
| | | EEVKNEMVGEKGLAPEVADRIGDYVQQHGGVSLVEQLLQDPKL | |
| | | SQNKQALEGLGDLKLLFEYLTLFGIDDKISFDLSLARGLDYYT | |
| | | GVIYEAVLLQTPAQAGEEPLGVGSVAAGGRYDGLVGMFDPKGR | |
| | | KVPCVGLSIGVERIFSIVEQRLEALEEKIRTTETQVLVASAQK | |
| | | KLLEERLKLVSELWDAGIKAELLYKKNPKLLNQLQYCEEAGIP | |
| | | LVAIIGEQELKDGVIKLRSVTSREEVDVRREDLVEEIKRRTGQ | |
| | | PLCIC | |
| HisRS1^{c8} | 1-60 + | MAERAALEELVKLQGERVRGLKQQKASAELIEEEVAKLLKLKA | 608 |
| | 399-509 | QLGPDESKQKFVLKTPKALEEKIRTTETQVLVASAQKKLLEER | |
| | | LKLVSELWDAGIKAELLYKKNPKLLNQLQYCEEAGIPLVAIIG | |
| | | EQELKDGVIKLRSVTSREEVDVRREDLVEEIKRRTGQPLCIC | |
| HisRS1^{c9} | 1-100 + | MAERAALEELVKLQGERVRGLKQQKASAELIEEEVAKLLKLKA | 609 |
| | 399-509 | QLGPDESKQKFVLKTPKGTRDYSPRQMAVREKVFDVIIRCFKR | |
| | | HGAEVIDTPVFELKALEEKIRTTETQVLVASAQKKLLEERLKL | |
| | | VSELWDAGIKAELLYKKNPKLLNQLQYCEEAGIPLVAIIGEQE | |
| | | LKDGVIKLRSVTSREEVDVRREDLVEEIKRRTGQPLCIC | |
| HisRS1^{c10} | 369-509 | MFDPKGRKVPCVGLSIGVERIFSIVEQRLEALEEKIRTTETQV | 610 |
| | | LVASAQKKLLEERLKLVSELWDAGIKAELLYKKNPKLLNQLQY | |
| | | CEEAGIPLVAIIGEQELKDGVIKLRSVTSREEVDVRREDLVEE | |
| | | IKRRTGQPLCIC | |
| HisRS1^{I1} | 191-333 | CLKIMCEILSSLQIGDFLVKVNDRRILDGMFAICGVSDSKFRT | 611 |
| | | ICSSVDKLDKVSWEEVKNEMVGEKGLAPEVADRIGDYVQQHGG | |
| | | VSLVEQLLQDPKLSQNKQALEGLGDLKLLFEYLTLFGIDDKIS | |
| | | FDLSLARGLDYYTG | |
| FL mito. | 1-506 | MPLLGLLPRRAWASLLSQLLRPPCASCTGAVRCQSQVAEAVLT | 612 |
| wild type | | SQLKAHQEKPNFIIKTPKGTRDLSPQHMVVREKILDLVISCFK | |
| | | RHGAKGMDTPAFELKETLTEKYGEDSGLMYDLKDQGGELLSLR | |
| | | YDLTVPFARYLAMNKVKKMKRYHVGKVWRRESPTIVQGRYREF | |
| | | CQCDFDIAGQFDPMIPDAECLKIMCEILSGLQLGDFLIKVNDR | |
| | | RIVDGMFAVCGVPESKFRAICSSIDKLDKMAWKDVRHEMVVKK | |
| | | GLAPEVADRIGDYVQCHGGVSLVEQMFQDPRLSQNKQALEGLG | |
| | | DLKLLFEYLTLFGIADKISFDLSLARGLDYYTGVIYEAVLLQT | |
| | | PTQAGEEPLNVGSVAAGGRYDGLVGMFDPKGHKVPCVGLSIGV | |
| | | ERIFYIVEQRMKTKGEKVRTTETQVFVATPQKNFLQERLKLIA | |
| | | ELWDSGIKAEMLYKNNPKLLTQLHYCESTGIPLVVIIGEQELK | |
| | | EGVIKIRSVASREEVAIKRENFVAEIQKRLSES | |
| | 152-398 | HVGKVWRRESPTIVQGRYREFCQCDFDIAGQFDPMIPDAECLK | 613 |
| | | IMCEILSGLQLGDFLIKVNDRRIVDGMFAVCGVPESKFRAICS | |
| | | SIDKLDKMAWKDVRHEMVVKKGLAPEVADRIGDYVQCHGGVSL | |
| | | VEQMFQDPRLSQNKQALEGLGDLKLLFEYLTLFGIADKISFDL | |
| | | SLARGLDYYTGVIYEAVLLQTPTQAGEEPLNVGSVAAGGRYDG | |
| | | LVGMFDPKGHKVPCVGLSIGVERIFYIVEQRM | |
| | 294-372 | QALEGLGDLKLLFEYLTLFGIDDKISFDLSLARGLDYYTGVIY | 614 |
| | | EAVLLQTPAQAGEEPLGVGSVAAGGRYDGLVGMFDP | |
| Amino- | 54-509 | FVLKTPKGTRDYSPRQMAVREKVFDVIIRCFKRHGAEVIDTPV | 615 |
| acylation | | FELKETLMGKYGEDSKLIYDLKDQGGELLSLRYDLTVPFARYL | |
| domain and | | AMNKLTNIKRYHIAKVYRRDNPAMTRGRYREFYQCDFDIAGNF | |
| anticodon | | DPMIPDAECLKIMCEILSSLQIGDFLVKVNDRRILDGMFAICG | |
| binding | | VSDSKFRTICSSVDKLDKVSWEEVKNEMVGEKGLAPEVADRIG | |
| domain | | DYVQQHGGVSLVEQLLQDPKLSQNKQALEGLGDLKLLFEYLTL | |
| | | FGIDDKISFDLSLARGLDYYTGVIYEAVLLQTPAQAGEEPLGV | |
| | | GSVAAGGRYDGLVGMFDPKGRKVPCVGLSIGVERIFSIVEQRL | |
| | | EALEEKIRTTETQVLVASAQKKLLEERLKLVSELWDAGIKAEL | |
| | | LYKKNPKLLNQLQYCEEAGIPLVAIIGEQELKDGVIKLRSVTS | |
| | | REEVDVRREDLVEEIKRRTGQPLCIC | |
| Amino- | 54-398 | FVLKTPKGTRDYSPRQMAVREKVFDVIIRCFKRHGAEVIDTPV | 616 |
| acylation | | FELKETLMGKYGEDSKLIYDLKDQGGELLSLRYDLTVPFARYL | |
| domain | | AMNKLTNIKRYHIAKVYRRDNPAMTRGRYREFYQCDFDIAGNF | |
| | | DPMIPDAECLKIMCEILSSLQIGDFLVKVNDRRILDGMFAICG | |
| | | VSDSKFRTICSSVDKLDKVSWEEVKNEMVGEKGLAPEVADRIG | |
| | | DYVQQHGGVSLVEQLLQDPKLSQNKQALEGLGDLKLLFEYLTL | |
| | | FGIDDKISFDLSLARGLDYYTGVIYEAVLLQTPAQAGEEPLGV | |
| | | GSVAAGGRYDGLVGMFDPKGRKVPCVGLSIGVERIFSIVEQRL | |
| | | E | |
| Amino- | 61-398 | GTRDYSPRQMAVREKVFDVIIRCFKRHGAEVIDTPVFELKETL | 617 |
| acylation | | MGKYGEDSKLIYDLKDQGGELLSLRYDLTVPFARYLAMNKLTN | |
| (core) | | IKRYHIAKVYRRDNPAMTRGRYREFYQCDFDIAGNFDPMIPDA | |
| domain | | ECLKIMCEILSSLQIGDFLVKVNDRRILDGMFAICGVSDSKFR | |
| | | TICSSVDKLDKVSWEEVKNEMVGEKGLAPEVADRIGDYVQQHG | |
| | | GVSLVEQLLQDPKLSQNKQALEGLGDLKLLFEYLTLFGIDDKI | |
| | | SFDLSLARGLDYYTGVIYEAVLLQTPAQAGEEPLGVGSVAAGG | |
| | | RYDGLVGMFDPKGRKVPCVGLSIGVERIFSIVEQRLE | |
| Anticodon | 399-509 | ALEEKIRTTETQVLVASAQKKLLEERLKLVSELWDAGIKAELL | 618 |
| binding | | YKKNPKLLNQLQYCEEAGIPLVAIIGEQELKDGVIKLRSVTSR | |
| domain | | EEVDVRREDLVEEIKRRTGQPLCIC | |
| Anticodon | 406-501 | TTETQVLVASAQKKLLEERLKLVSELWDAGIKAELLYKKNPKL | 619 |
| binding | | LNQLQYCEEAGIPLVAIIGEQELKDGVIKLRSVTSREEVDVRR | |
| (core) | | EDLVEEIKRR | |
| domain | | | |
| | 399-500 | ALEEKIRTTETQVLVASAQKKLLEERLKLVSELWDAGIKAELL | 620 |
| | | YKKNPKLLNQLQYCEEAGIPLVAIIGEQELKDGVIKLRSVTSR | |
| | | EEVDVRREDLVEEIKR | |
| | 399-499 | ALEEKIRTTETQVLVASAQKKLLEERLKLVSELWDAGIKAELL | 621 |
| | | YKKNPKLLNQLQYCEEAGIPLVAIIGEQELKDGVIKLRSVTSR | |
| | | EEVDVRREDLVEEIK | |
| | 399-498 | ALEEKIRTTETQVLVASAQKKLLEERLKLVSELWDAGIKAELL | 622 |
| | | YKKNPKLLNQLQYCEEAGIPLVAIIGEQELKDGVIKLRSVTSR | |
| | | EEVDVRREDLVEEI | |
| | 399-497 | ALEEKIRTTETQVLVASAQKKLLEERLKLVSELWDAGIKAELL | 623 |
| | | YKKNPKLLNQLQYCEEAGIPLVAIIGEQELKDGVIKLRSVTSR | |
| | | EEVDVRREDLVEE | |
| | 399-496 | ALEEKIRTTETQVLVASAQKKLLEERLKLVSELWDAGIKAELL | 624 |
| | | YKKNPKLLNQLQYCEEAGIPLVAIIGEQELKDGVIKLRSVTSR | |
| | | EEVDVRREDLVE | |
| | 399-495 | ALEEKIRTTETQVLVASAQKKLLEERLKLVSELWDAGIKAELL | 625 |
| | | YKKNPKLLNQLQYCEEAGIPLVAIIGEQELKDGVIKLRSVTSR | |
| | | EEVDVRREDLV | |
| | 399-494 | ALEEKIRTTETQVLVASAQKKLLEERLKLVSELWDAGIKAELL | 626 |
| | | YKKNPKLLNQLQYCEEAGIPLVAIIGEQELKDGVIKLRSVTSR | |
| | | EEVDVRREDL | |
| | 399-493 | ALEEKIRTTETQVLVASAQKKLLEERLKLVSELWDAGIKAELL | 627 |
| | | YKKNPKLLNQLQYCEEAGIPLVAIIGEQELKDGVIKLRSVTSR | |
| | | EEVDVRRED | |
| | 399-492 | ALEEKIRTTETQVLVASAQKKLLEERLKLVSELWDAGIKAELL | 628 |
| | | YKKNPKLLNQLQYCEEAGIPLVAIIGEQELKDGVIKLRSVTSR | |
| | | EEVDVRRED | |
| | 399-491 | ALEEKIRTTETQVLVASAQKKLLEERLKLVSELWDAGIKAELL | 629 |
| | | YKKNPKLLNQLQYCEEAGIPLVAIIGEQELKDGVIKLRSVTSR | |
| | | EEVDVRRE | |
| | 399-490 | ALEEKIRTTETQVLVASAQKKLLEERLKLVSELWDAGIKAELL | 630 |
| | | YKKNPKLLNQLQYCEEAGIPLVAIIGEQELKDGVIKLRSVTSR | |
| | | EEVDVRR | |
| | 399-489 | ALEEKIRTTETQVLVASAQKKLLEERLKLVSELWDAGIKAELL | 631 |
| | | YKKNPKLLNQLQYCEEAGIPLVAIIGEQELKDGVIKLRSVTSR | |
| | | EEVDVR | |
| | 399-488 | ALEEKIRTTETQVLVASAQKKLLEERLKLVSELWDAGIKAELL | 632 |
| | | YKKNPKLLNQLQYCEEAGIPLVAIIGEQELKDGVIKLRSVTSR | |
| | | EEVDV | |
| | 399-487 | ALEEKIRTTETQVLVASAQKKLLEERLKLVSELWDAGIKAELL | 633 |
| | | YKKNPKLLNQLQYCEEAGIPLVAIIGEQELKDGVIKLRSVTSR | |
| | | EEVD | |
| | 399-486 | ALEEKIRTTETQVLVASAQKKLLEERLKLVSELWDAGIKAELL | 634 |
| | | YKKNPKLLNQLQYCEEAGIPLVAIIGEQELKDGVIKLRSVTSR | |
| | | EEV | |
| | 399-485 | ALEEKIRTTETQVLVASAQKKLLEERLKLVSELWDAGIKAELL | 635 |
| | | YKKNPKLLNQLQYCEEAGIPLVAIIGEQELKDGVIKLRSVTSR | |
| | | EE | |
| | 399-484 | ALEEKIRTTETQVLVASAQKKLLEERLKLVSELWDAGIKAELL | 636 |
| | | YKKNPKLLNQLQYCEEAGIPLVAIIGEQELKDGVIKLRSVTSR | |
| | | E | |
| | 399-483 | ALEEKIRTTETQVLVASAQKKLLEERLKLVSELWDAGIKAELL | 637 |
| | | YKKNPKLLNQLQYCEEAGIPLVAIIGEQELKDGVIKLRSVTSR | |
| | 399-482 | ALEEKIRTTETQVLVASAQKKLLEERLKLVSELWDAGIKAELL | 638 |
| | | YKKNPKLLNQLQYCEEAGIPLVAIIGEQELKDGVIKLRSVTS | |
| | 399-481 | ALEEKIRTTETQVLVASAQKKLLEERLKLVSELWDAGIKAELL | 639 |
| | | YKKNPKLLNQLQYCEEAGIPLVAIIGEQELKDGVIKLRSVT | |
| | 399-480 | ALEEKIRTTETQVLVASAQKKLLEERLKLVSELWDAGIKAELL | 640 |
| | | YKKNPKLLNQLQYCEEAGIPLVAIIGEQELKDGVIKLRSV | |
| | 399-479 | ALEEKIRTTETQVLVASAQKKLLEERLKLVSELWDAGIKAELL | 641 |
| | | YKKNPKLLNQLQYCEEAGIPLVAIIGEQELKDGVIKLRS | |
| | 399-478 | ALEEKIRTTETQVLVASAQKKLLEERLKLVSELWDAGIKAELL | 642 |
| | | YKKNPKLLNQLQYCEEAGIPLVAIIGEQELKDGVIKLR | |
| | 399-477 | ALEEKIRTTETQVLVASAQKKLLEERLKLVSELWDAGIKAELL | 643 |
| | | YKKNPKLLNQLQYCEEAGIPLVAIIGEQELKDGVIKL | |
| | 399-476 | ALEEKIRTTETQVLVASAQKKLLEERLKLVSELWDAGIKAELL | 644 |
| | | YKKNPKLLNQLQYCEEAGIPLVAIIGEQELKDGVIK | |
| | 399-475 | ALEEKIRTTETQVLVASAQKKLLEERLKLVSELWDAGIKAELL | 645 |
| | | YKKNPKLLNQLQYCEEAGIPLVAIIGEQELKDGVI | |
| | 399-474 | ALEEKIRTTETQVLVASAQKKLLEERLKLVSELWDAGIKAELL | 646 |
| | | YKKNPKLLNQLQYCEEAGIPLVAIIGEQELKDGV | |
| | 399-473 | ALEEKIRTTETQVLVASAQKKLLEERLKLVSELWDAGIKAELL | 647 |
| | | YKKNPKLLNQLQYCEEAGIPLVAIIGEQELKDG | |
| | 399-472 | ALEEKIRTTETQVLVASAQKKLLEERLKLVSELWDAGIKAELL | 648 |
| | | YKKNPKLLNQLQYCEEAGIPLVAIIGEQELKD | |
| | 399-471 | ALEEKIRTTETQVLVASAQKKLLEERLKLVSELWDAGIKAELL | 649 |
| | | YKKNPKLLNQLQYCEEAGIPLVAIIGEQELK | |
| | 399-470 | ALEEKIRTTETQVLVASAQKKLLEERLKLVSELWDAGIKAELL | 650 |
| | | YKKNPKLLNQLQYCEEAGIPLVAIIGEQEL | |
| | 399-469 | ALEEKIRTTETQVLVASAQKKLLEERLKLVSELWDAGIKAELL | 651 |
| | | YKKNPKLLNQLQYCEEAGIPLVAIIGEQE | |
| | 399-468 | ALEEKIRTTETQVLVASAQKKLLEERLKLVSELWDAGIKAELL | 652 |
| | | YKKNPKLLNQLQYCEEAGIPLVAIIGEQ | |
| | 399-467 | ALEEKIRTTETQVLVASAQKKLLEERLKLVSELWDAGIKAELL | 653 |
| | | YKKNPKLLNQLQYCEEAGIPLVAIIGE | |
| | 399-466 | ALEEKIRTTETQVLVASAQKKLLEERLKLVSELWDAGIKAELL | 654 |
| | | YKKNPKLLNQLQYCEEAGIPLVAIIG | |
| | 399-465 | ALEEKIRTTETQVLVASAQKKLLEERLKLVSELWDAGIKAELL | 655 |
| | | YKKNPKLLNQLQYCEEAGIPLVAII | |
| | 399-464 | ALEEKIRTTETQVLVASAQKKLLEERLKLVSELWDAGIKAELL | 656 |
| | | YKKNPKLLNQLQYCEEAGIPLVAI | |
| | 399-463 | ALEEKIRTTETQVLVASAQKKLLEERLKLVSELWDAGIKAELL | 657 |
| | | YKKNPKLLNQLQYCEEAGIPLVA | |
| | 399-462 | ALEEKIRTTETQVLVASAQKKLLEERLKLVSELWDAGIKAELL | 658 |
| | | YKKNPKLLNQLQYCEEAGIPLV | |
| | 399-461 | ALEEKIRTTETQVLVASAQKKLLEERLKLVSELWDAGIKAELL | 659 |
| | | YKKNPKLLNQLQYCEEAGIPL | |
| | 399-460 | ALEEKIRTTETQVLVASAQKKLLEERLKLVSELWDAGIKAELL | 660 |
| | | YKKNPKLLNQLQYCEEAGIP | |
| | 399-459 | ALEEKIRTTETQVLVASAQKKLLEERLKLVSELWDAGIKAELL | 661 |
| | | YKKNPKLLNQLQYCEEAGI | |
| | 399-458 | ALEEKIRTTETQVLVASAQKKLLEERLKLVSELWDAGIKAELL | 662 |
| | | YKKNPKLLNQLQYCEEAG | |
| | 399-457 | ALEEKIRTTETQVLVASAQKKLLEERLKLVSELWDAGIKAELL | 663 |
| | | YKKNPKLLNQLQYCEEA | |
| | 399-456 | ALEEKIRTTETQVLVASAQKKLLEERLKLVSELWDAGIKAELL | 664 |
| | | YKKNPKLLNQLQYCEE | |
| | 399-455 | ALEEKIRTTETQVLVASAQKKLLEERLKLVSELWDAGIKAELL | 665 |
| | | YKKNPKLLNQLQYCE | |
| | 399-454 | ALEEKIRTTETQVLVASAQKKLLEERLKLVSELWDAGIKAELL | 666 |
| | | YKKNPKLLNQLQYC | |
| | 399-453 | ALEEKIRTTETQVLVASAQKKLLEERLKLVSELWDAGIKAELL | 667 |
| | | YKKNPKLLNQLQY | |
| | 399-452 | ALEEKIRTTETQVLVASAQKKLLEERLKLVSELWDAGIKAELL | 668 |
| | | YKKNPKLLNQLQ | |
| | 399-451 | ALEEKIRTTETQVLVASAQKKLLEERLKLVSELWDAGIKAELL | 669 |
| | | YKKNPKLLNQL | |
| | 399-450 | ALEEKIRTTETQVLVASAQKKLLEERLKLVSELWDAGIKAELL | 670 |
| | | YKKNPKLLNQ | |
| | 399-449 | ALEEKIRTTETQVLVASAQKKLLEERLKLVSELWDAGIKAELL | 671 |
| | | YKKNPKLLN | |
| | 399-448 | ALEEKIRTTETQVLVASAQKKLLEERLKLVSELWDAGIKAELL | 672 |
| | | YKKNPKLL | |
| | 399-447 | ALEEKIRTTETQVLVASAQKKLLEERLKLVSELWDAGIKAELL | 673 |
| | | YKKNPKL | |
| | 399-446 | ALEEKIRTTETQVLVASAQKKLLEERLKLVSELWDAGIKAELL | 674 |
| | | YKKNPK | |
| | 399-445 | ALEEKIRTTETQVLVASAQKKLLEERLKLVSELWDAGIKAELL | 675 |
| | | YKKNP | |
| | 399-444 | ALEEKIRTTETQVLVASAQKKLLEERLKLVSELWDAGIKAELL | 676 |
| | | YKKN | |
| | 399-443 | ALEEKIRTTETQVLVASAQKKLLEERLKLVSELWDAGIKAELL | 677 |
| | | YKK | |
| | 399-442 | ALEEKIRTTETQVLVASAQKKLLEERLKLVSELWDAGIKAELL | 678 |
| | | YK | |
| | 399-441 | ALEEKIRTTETQVLVASAQKKLLEERLKLVSELWDAGIKAELL | 679 |
| | | Y | |
| | 399-440 | ALEEKIRTTETQVLVASAQKKLLEERLKLVSELWDAGIKAELL | 680 |
| | 399-439 | ALEEKIRTTETQVLVASAQKKLLEERLKLVSELWDAGIKAEL | 681 |
| | 399-438 | ALEEKIRTTETQVLVASAQKKLLEERLKLVSELWDAGIKAE | 682 |
| | 399-437 | ALEEKIRTTETQVLVASAQKKLLEERLKLVSELWDAGIKA | 683 |
| | 399-436 | ALEEKIRTTETQVLVASAQKKLLEERLKLVSELWDAGIK | 684 |
| | 399-435 | ALEEKIRTTETQVLVASAQKKLLEERLKLVSELWDAGI | 685 |
| | 399-434 | ALEEKIRTTETQVLVASAQKKLLEERLKLVSELWDAG | 686 |
| | 399-433 | ALEEKIRTTETQVLVASAQKKLLEERLKLVSELWDA | 687 |
| | 399-432 | ALEEKIRTTETQVLVASAQKKLLEERLKLVSELWD | 688 |
| | 399-431 | ALEEKIRTTETQVLVASAQKKLLEERLKLVSELW | 689 |
| | 399-430 | ALEEKIRTTETQVLVASAQKKLLEERLKLVSEL | 690 |
| | 399-429 | ALEEKIRTTETQVLVASAQKKLLEERLKLVSE | 691 |
| | 399-428 | ALEEKIRTTETQVLVASAQKKLLEERLKLVS | 692 |
| | 399-427 | ALEEKIRTTETQVLVASAQKKLLEERLKLV | 693 |
| | 399-426 | ALEEKIRTTETQVLVASAQKKLLEERLKL | 694 |
| | 399-425 | ALEEKIRTTETQVLVASAQKKLLEERLK | 695 |
| | 399-424 | ALEEKIRTTETQVLVASAQKKLLEERL | 696 |
| | 399-423 | ALEEKIRTTETQVLVASAQKKLLEER | 697 |
| | 399-422 | ALEEKIRTTETQVLVASAQKKLLEE | 698 |
| | 399-421 | ALEEKIRTTETQVLVASAQKKLLE | 699 |
| | 399-420 | ALEEKIRTTETQVLVASAQKKLL | 700 |
| | 399-419 | ALEEKIRTTETQVLVASAQKKL | 701 |
| | 400-501 | LEEKIRTTETQVLVASAQKKLLEERLKLVSELWDAGIKAELLY | 702 |
| | | KKNPKLLNQLQYCEEAGIPLVAIIGEQELKDGVIKLRSVTSRE | |
| | | EVDVRREDLVEEIKRR | |
| | 401-501 | EEKIRTTETQVLVASAQKKLLEERLKLVSELWDAGIKAELLYK | 703 |
| | | KNPKLLNQLQYCEEAGIPLVAIIGEQELKDGVIKLRSVTSREE | |
| | | VDVRREDLVEEIKRR | |
| | 402-501 | EKIRTTETQVLVASAQKKLLEERLKLVSELWDAGIKAELLYKK | 704 |
| | | NPKLLNQLQYCEEAGIPLVAIIGEQELKDGVIKLRSVTSREEV | |
| | | DVRREDLVEEIKRR | |
| | 403-501 | KIRTTETQVLVASAQKKLLEERLKLVSELWDAGIKAELLYKKN | 705 |
| | | PKLLNQLQYCEEAGIPLVAIIGEQELKDGVIKLRSVTSREEVD | |
| | | VRREDLVEEIKRR | |
| | 404-501 | RTTETQVLVASAQKKLLEERLKLVSELWDAGIKAELLYKKNPK | 706 |
| | | LLNQLQYCEEAGIPLVAIIGEQELKDGVIKLRSVTSREEVDVR | |
| | | REDLVEEIKRR | |
| | 405-501 | TTETQVLVASAQKKLLEERLKLVSELWDAGIKAELLYKKNPKL | 707 |
| | | LNQLQYCEEAGIPLVAIIGEQELKDGVIKLRSVTSREEVDVRR | |
| | | EDLVEEIKRR | |
| | 406-501 | TETQVLVASAQKKLLEERLKLVSELWDAGIKAELLYKKNPKLL | 708 |
| | | NQLQYCEEAGIPLVAIIGEQELKDGVIKLRSVTSREEVDVRRE | |
| | | DLVEEIKRR | |
| | 407-501 | ETQVLVASAQKKLLEERLKLVSELWDAGIKAELLYKKNPKLLN | 709 |
| | | QLQYCEEAGIPLVAIIGEQELKDGVIKLRSVTSREEVDVRRED | |
| | | LVEEIKRR | |
| | 408-501 | TQVLVASAQKKLLEERLKLVSELWDAGIKAELLYKKNPKLLNQ | 710 |
| | | LQYCEEAGIPLVAIIGEQELKDGVIKLRSVTSREEVDVRREDL | |
| | | VEEIKRR | |
| | 409-501 | QVLVASAQKKLLEERLKLVSELWDAGIKAELLYKKNPKLLNQL | 711 |
| | | QYCEEAGIPLVAIIGEQELKDGVIKLRSVTSREEVDVRREDLV | |
| | | EEIKRR | |
| | 410-501 | VLVASAQKKLLEERLKLVSELWDAGIKAELLYKKNPKLLNQLQ | 712 |
| | | YCEEAGIPLVAIIGEQELKDGVIKLRSVTSREEVDVRREDLVE | |
| | | EIKRR | |
| | 411-501 | LVASAQKKLLEERLKLVSELWDAGIKAELLYKKNPKLLNQLQY | 713 |
| | | CEEAGIPLVAIIGEQELKDGVIKLRSVTSREEVDVRREDLVEE | |
| | | IKRR | |
| | 412-501 | VASAQKKLLEERLKLVSELWDAGIKAELLYKKNPKLLNQLQYC | 714 |
| | | EEAGIPLVAIIGEQELKDGVIKLRSVTSREEVDVRREDLVEEI | |
| | | KRR | |
| | 413-501 | ASAQKKLLEERLKLVSELWDAGIKAELLYKKNPKLLNQLQYCE | 715 |
| | | EAGIPLVAIIGEQELKDGVIKLRSVTSREEVDVRREDLVEEIK | |
| | | RR | |
| | 414-501 | SAQKKLLEERLKLVSELWDAGIKAELLYKKNPKLLNQLQYCEE | 716 |
| | | AGIPLVAIIGEQELKDGVIKLRSVTSREEVDVRREDLVEEIKR | |
| | | R | |
| | 415-501 | AQKKLLEERLKLVSELWDAGIKAELLYKKNPKLLNQLQYCEEA | 717 |
| | | GIPLVAIIGEQELKDGVIKLRSVTSREEVDVRREDLVEEIKRR | |
| | 416-501 | QKKLLEERLKLVSELWDAGIKAELLYKKNPKLLNQLQYCEEAG | 718 |
| | | IPLVAIIGEQELKDGVIKLRSVTSREEVDVRREDLVEEIKRR | |
| | 417-501 | KKLLEERLKLVSELWDAGIKAELLYKKNPKLLNQLQYCEEAGI | 719 |
| | | PLVAIIGEQELKDGVIKLRSVTSREEVDVRREDLVEEIKRR | |
| | 418-501 | KLLEERLKLVSELWDAGIKAELLYKKNPKLLNQLQYCEEAGIP | 720 |
| | | LVAIIGEQELKDGVIKLRSVTSREEVDVRREDLVEEIKRR | |
| | 419-501 | LLEERLKLVSELWDAGIKAELLYKKNPKLLNQLQYCEEAGIPL | 721 |
| | | VAIIGEQELKDGVIKLRSVTSREEVDVRREDLVEEIKRR | |
| | 420-501 | LEERLKLVSELWDAGIKAELLYKKNPKLLNQLQYCEEAGIPLV | 722 |
| | | AIIGEQELKDGVIKLRSVTSREEVDVRREDLVEEIKRR | |
| | 421-501 | EERLKLVSELWDAGIKAELLYKKNPKLLNQLQYCEEAGIPLVA | 723 |
| | | IIGEQELKDGVIKLRSVTSREEVDVRREDLVEEIKRR | |
| | 422-501 | ERLKLVSELWDAGIKAELLYKKNPKLLNQLQYCEEAGIPLVAI | 724 |
| | | IGEQELKDGVIKLRSVTSREEVDVRREDLVEEIKRR | |
| | 423-501 | RLKLVSELWDAGIKAELLYKKNPKLLNQLQYCEEAGIPLVAII | 725 |
| | | GEQELKDGVIKLRSVTSREEVDVRREDLVEEIKRR | |
| | 424-501 | LKLVSELWDAGIKAELLYKKNPKLLNQLQYCEEAGIPLVAIIG | 726 |
| | | EQELKDGVIKLRSVTSREEVDVRREDLVEEIKRR | |
| | 425-501 | KLVSELWDAGIKAELLYKKNPKLLNQLQYCEEAGIPLVAIIGE | 727 |
| | | QELKDGVIKLRSVTSREEVDVRREDLVEEIKRR | |
| | 426-501 | LVSELWDAGIKAELLYKKNPKLLNQLQYCEEAGIPLVAIIGEQ | 728 |
| | | ELKDGVIKLRSVTSREEVDVRREDLVEEIKRR | |
| | 427-501 | VSELWDAGIKAELLYKKNPKLLNQLQYCEEAGIPLVAIIGEQE | 729 |
| | | LKDGVIKLRSVTSREEVDVRREDLVEEIKRR | |
| | 428-501 | SELWDAGIKAELLYKKNPKLLNQLQYCEEAGIPLVAIIGEQEL | 730 |
| | | KDGVIKLRSVTSREEVDVRREDLVEEIKRR | |
| | 429-501 | ELWDAGIKAELLYKKNPKLLNQLQYCEEAGIPLVAIIGEQELK | 731 |
| | | DGVIKLRSVTSREEVDVRREDLVEEIKRR | |
| | 430-501 | LWDAGIKAELLYKKNPKLLNQLQYCEEAGIPLVAIIGEQELKD | 732 |
| | | GVIKLRSVTSREEVDVRREDLVEEIKRR | |
| | 431-501 | WDAGIKAELLYKKNPKLLNQLQYCEEAGIPLVAIIGEQELKDG | 733 |
| | | VIKLRSVTSREEVDVRREDLVEEIKRR | |
| | 432-501 | DAGIKAELLYKKNPKLLNQLQYCEEAGIPLVAIIGEQELKDGV | 734 |
| | | IKLRSVTSREEVDVRREDLVEEIKRR | |
| | 433-501 | AGIKAELLYKKNPKLLNQLQYCEEAGIPLVAIIGEQELKDGVI | 735 |
| | | KLRSVTSREEVDVRREDLVEEIKRR | |
| | 434-501 | GIKAELLYKKNPKLLNQLQYCEEAGIPLVAIIGEQELKDGVIK | 736 |
| | | LRSVTSREEVDVRREDLVEEIKRR | |
| | 435-501 | IKAELLYKKNPKLLNQLQYCEEAGIPLVAIIGEQELKDGVIKL | 737 |
| | | RSVTSREEVDVRREDLVEEIKRR | |
| | 436-501 | KAELLYKKNPKLLNQLQYCEEAGIPLVAIIGEQELKDGVIKLR | 738 |
| | | SVTSREEVDVRREDLVEEIKRR | |
| | 437-501 | AELLYKKNPKLLNQLQYCEEAGIPLVAIIGEQELKDGVIKLRS | 739 |
| | | VTSREEVDVRREDLVEEIKRR | |
| | 438-501 | ELLYKKNPKLLNQLQYCEEAGIPLVAIIGEQELKDGVIKLRSV | 740 |
| | | TSREEVDVRREDLVEEIKRR | |
| | 439-501 | LLYKKNPKLLNQLQYCEEAGIPLVAIIGEQELKDGVIKLRSVT | 741 |
| | | SREEVDVRREDLVEEIKRR | |
| | 440-501 | LYKKNPKLLNQLQYCEEAGIPLVAIIGEQELKDGVIKLRSVTS | 742 |
| | | REEVDVRREDLVEEIKRR | |
| | 441-501 | YKKNPKLLNQLQYCEEAGIPLVAIIGEQELKDGVIKLRSVTSR | 743 |
| | | EEVDVRREDLVEEIKRR | |
| | 442-501 | KKNPKLLNQLQYCEEAGIPLVAIIGEQELKDGVIKLRSVTSRE | 744 |
| | | EVDVRREDLVEEIKRR | |
| | 443-501 | KNPKLLNQLQYCEEAGIPLVAIIGEQELKDGVIKLRSVTSREE | 745 |
| | | VDVRREDLVEEIKRR | |
| | 444-501 | NPKLLNQLQYCEEAGIPLVAIIGEQELKDGVIKLRSVTSREEV | 746 |
| | | DVRREDLVEEIKRR | |
| | 445-501 | PKLLNQLQYCEEAGIPLVAIIGEQELKDGVIKLRSVTSREEVD | 747 |
| | | VRREDLVEEIKRR | |
| | 446-501 | KLLNQLQYCEEAGIPLVAIIGEQELKDGVIKLRSVTSREEVDV | 748 |
| | | RREDLVEEIKRR | |
| | 447-501 | LLNQLQYCEEAGIPLVAIIGEQELKDGVIKLRSVTSREEVDVR | 749 |
| | | REDLVEEIKRR | |
| | 448-501 | LNQLQYCEEAGIPLVAIIGEQELKDGVIKLRSVTSREEVDVRR | 750 |
| | | EDLVEEIKRR | |
| | 449-501 | NQLQYCEEAGIPLVAIIGEQELKDGVIKLRSVTSREEVDVRRE | 751 |
| | | DLVEEIKRR | |
| | 450-501 | QLQYCEEAGIPLVAIIGEQELKDGVIKLRSVTSREEVDVRRED | 752 |
| | | LVEEIKRR | |
| | 451-501 | LQYCEEAGIPLVAIIGEQELKDGVIKLRSVTSREEVDVRREDL | 753 |
| | | VEEIKRR | |
| | 452-501 | QYCEEAGIPLVAIIGEQELKDGVIKLRSVTSREEVDVRREDLV | 754 |
| | | EEIKRR | |
| | 453-501 | YCEEAGIPLVAIIGEQELKDGVIKLRSVTSREEVDVRREDLVE | 755 |
| | | EIKRR | |
| | 454-501 | CEEAGIPLVAIIGEQELKDGVIKLRSVTSREEVDVRREDLVEE | 756 |
| | | IKRR | |
| | 455-501 | EEAGIPLVAIIGEQELKDGVIKLRSVTSREEVDVRREDLVEEI | 757 |
| | | KRR | |
| | 456-501 | EAGIPLVAIIGEQELKDGVIKLRSVTSREEVDVRREDLVEEIK | 758 |
| | | RR | |
| | 457-501 | AGIPLVAIIGEQELKDGVIKLRSVTSREEVDVRREDLVEEIKR | 759 |
| | | R | |
| | 458-501 | GIPLVAIIGEQELKDGVIKLRSVTSREEVDVRREDLVEEIKRR | 760 |
| | 459-501 | IPLVAIIGEQELKDGVIKLRSVTSREEVDVRREDLVEEIKRR | 761 |
| | 460-501 | PLVAIIGEQELKDGVIKLRSVTSREEVDVRREDLVEEIKRR | 762 |
| | 461-501 | LVAIIGEQELKDGVIKLRSVTSREEVDVRREDLVEEIKRR | 763 |
| | 462-501 | VAIIGEQELKDGVIKLRSVTSREEVDVRREDLVEEIKRR | 764 |
| | 463-501 | AIIGEQELKDGVIKLRSVTSREEVDVRREDLVEEIKRR | 765 |
| | 464-501 | IIGEQELKDGVIKLRSVTSREEVDVRREDLVEEIKRR | 766 |
| | 465-501 | IGEQELKDGVIKLRSVTSREEVDVRREDLVEEIKRR | 767 |
| | 466-501 | GEQELKDGVIKLRSVTSREEVDVRREDLVEEIKRR | 768 |
| | 467-501 | EQELKDGVIKLRSVTSREEVDVRREDLVEEIKRR | 769 |
| | 468-501 | QELKDGVIKLRSVTSREEVDVRREDLVEEIKRR | 770 |
| | 469-501 | ELKDGVIKLRSVTSREEVDVRREDLVEEIKRR | 771 |
| | 470-501 | LKDGVIKLRSVTSREEVDVRREDLVEEIKRR | 772 |
| | 471-501 | KDGVIKLRSVTSREEVDVRREDLVEEIKRR | 773 |
| | 472-501 | DGVIKLRSVTSREEVDVRREDLVEEIKRR | 774 |
| | 473-501 | GVIKLRSVTSREEVDVRREDLVEEIKRR | 775 |
| | 474-501 | VIKLRSVTSREEVDVRREDLVEEIKRR | 776 |
| | 475-501 | IKLRSVTSREEVDVRREDLVEEIKRR | 777 |
| | 476-501 | KLRSVTSREEVDVRREDLVEEIKRR | 778 |
| | 477-501 | LRSVTSREEVDVRREDLVEEIKRR | 779 |
| | 478-501 | RSVTSREEVDVRREDLVEEIKRR | 780 |
| | 479-501 | SVTSREEVDVRREDLVEEIKRR | 781 |
| | 480-501 | VTSREEVDVRREDLVEEIKRR | 782 |
| | 481-501 | TSREEVDVRREDLVEEIKRR | 783 |
| | 13-35 | LQGERVRGLKQQKASAELIEEEV | 784 |
| Splice Jn. | | KFVLKTPK | 785 |
| Splice Jn. | | SSVDKLDKVGYPWWNS | 786 |
| Splice Jn. | | KFVLKTPKDFDIAGNF | 787 |
| Splice Jn. | | KFVLKTPKVNDRRILD | 788 |
| Splice Jn. | | DTPVFELKVNDRRILD | 789 |
| Splice Jn. | | RYREFYQCVNDRRILD | 790 |
| Splice Jn. | | KFVLKTPKETLMGKYG | 791 |
| Splice Jn. | | DTPVFELKDFDIAGNF | 792 |
| Splice Jn. | | KFVLKTPKALEEKIRT | 793 |
| Splice Jn. | | DTPVFELKALEEKIRT | 794 |
| HRS WHEP | | X_{A}-L-X_{B}-Q-G-X-X-V-R-X-L-K-X-X-K-A-X_{c}-V-X-X- | 795 |
| consensus | | L-L-X-L-K-X_{D} | |
| | | Where: | |
| | | X is any amino acid | |
| | | X_{A} is 0-50 amino acids | |
| | | X_{B} is about 5-7 amino acids, | |
| | | preferably 6 amino acids | |
| | | X_{c} is about 7-9 amino acids, | |
| | | preferably 8 amino acids | |
| | | X_{D} is 0-50 amino acids | |

Accordingly, in certain embodiments, an antibody or antigen-binding fragment thereof specifically binds to an HRS polypeptide that comprises, consists, or consists essentially of an amino acid sequence in Table H1 (SEQ ID NO:) or a variant, fragment, or epitope thereof, and/or a complex comprising the HRS polypeptide.

In some embodiments, an antibody or antigen-binding fragment thereof inhibits, blocks, or otherwise interferes with the binding of a human HRS polypeptide to a human neuropilin-2 (NP2 or NRP2) polypeptide (and vice versa). Exemplary isoforms of human NP2 are provided in Table N1, and exemplary HRS polypeptides are provided in Table H1.

TABLE N1
Exemplary Human neuropilin polypeptides

| | | | SEQ ID |
|---|---|---|---|
| Name | Residues | Sequence | NO: |
| FL | 23-926 | QPDPPCGGRLNSKDAGYITSPGYPQDYPSHQNCEWIVYAPEPN | 796 |
| neuropilin | | QKIVLNFNPHFEIEKHDCKYDFIEIRDGDSESADLLGKHCGNI | |
| 2 splice | | APPTIISSGSMLYIKFTSDYARQGAGFSLRYEIFKTGSEDCSK | |
| variant 2 | | NFTSPNGTIESPGFPEKYPHNLDCTFTILAKPKMEIILQFLIF | |
| | | DLEHDPLQVGEGDCKYDWLDIWDGIPHVGPLIGKYCGTKTPSE | |
| | | LRSSTGILSLTFHTDMAVAKDGFSARYYLVHQEPLENFQCNVP | |
| | | LGMESGRIANEQISASSTYSDGRWTPQQSRLHGDDNGWTPNLD | |
| | | SNKEYLQVDLRFLTMLTAIATQGAISRETQNGYYVKSYKLEVS | |
| | | TNGEDWMVYRHGKNHKVFQANNDATEVVLNKLHAPLLTREVRI | |
| | | RPQTWHSGIALRLELFGCRVTDAPCSNMLGMLSGLIADSQISA | |
| | | SSTQEYLWSPSAARLVSSRSGWFPRIPQAQPGEEWLQVDLGTP | |
| | | KTVKGVIIQGARGGDSITAVEARAFVRKFKVSYSLNGKDWEYI | |
| | | QDPRTQQPKLFEGNMHYDTPDIRRFDPIPAQYVRVYPERWSPA | |
| | | GIGMRLEVLGCDWTDSKPTVETLGPTVKSEETTTPYPTEEEAT | |
| | | ECGENCSFEDDKDLQLPSGFNCNFDFLEEPCGWMYDHAKWLRT | |
| | | TWASSSSPNDRTFPDDRNFLRLQSDSQREGQYARLISPPVHLP | |
| | | RSPVCMEFQYQATGGRGVALQVVREASQESKLLWVIREDQGGE | |
| | | WKHGRIILPSYDMEYQIVFEGVIGKGRSGEIAIDDIRISTDVP | |
| | | LENCMEPISAFAVDIPEIHEREGYEDEIDDEYEVDWSNSSSAT | |
| | | SGSGAPSTDKEKSWLYTLDPILITIIAMSSLGVLLGATCAGLL | |
| | | LYCTCSYSGLSSRSCTTLENYNFELYDGLKHKVKMNHQKCCSE | |
| | | A | |
| Neuropilin | 23-901 | QPDPPCGGRLNSKDAGYITSPGYPQDYPSHQNCEWIVYAPEPN | 797 |
| 2 splice | | QKIVLNFNPHFEIEKHDCKYDFIEIRDGDSESADLLGKHCGNI | |
| variant 5 | | APPTIISSGSMLYIKFTSDYARQGAGFSLRYEIFKTGSEDCSK | |
| | | NFTSPNGTIESPGFPEKYPHNLDCTFTILAKPKMEIILQFLIF | |
| | | DLEHDPLQVGEGDCKYDWLDIWDGIPHVGPLIGKYCGTKTPSE | |
| | | LRSSTGILSLIFHTDMAVAKDGFSARYYLVHQEPLENFQCNVP | |
| | | LGMESGRIANEQISASSTYSDGRWTPQQSRLHGDDNGWTPNLD | |
| | | SNKEYLQVDLRFLTMLTAIATQGAISRETQNGYYVKSYKLEVS | |
| | | TNGEDWMVYRHGKNHKVFQANNDATEVVLNKLHAPLLTREVRI | |
| | | RPQTWHSGIALRLELFGCRVTDAPCSNMLGMLSGLIADSQISA | |
| | | SSTQEYLWSPSAARLVSSRSGWFPRIPQAQPGEEWLQVDLGTP | |
| | | KTVKGVIIQGARGGDSITAVEARAFVRKFKVSYSLNGKDWEYI | |
| | | QDPRTQQPKLFEGNMHYDTPDIRRFDPIPAQYVRVYPERWSPA | |
| | | GIGMRLEVLGCDWTDSKPTVETLGPTVKSEETTTPYPTEEEAT | |
| | | ECGENCSFEDDKDLQLPSGFNCNFDFLEEPCGWMYDHAKWLRT | |
| | | TWASSSSPNDRTFPDDRNFLRLQSDSQREGQYARLISPPVHLP | |
| | | RSPVCMEFQYQATGGRGVALQVVREASQESKLLWVIREDQGGE | |
| | | WKHGRIILPSYDMEYQIVFEGVIGKGRSGEIAIDDIRISTDVP | |
| | | LENCMEPISAFAGGTLLPGTEPTVDTVPMQPIPAYWYYVMAAG | |
| | | GAVLVLVSVALALVLHYHRFRYAAKKTDHSITYKTSHYTNGAP | |
| | | LAVEPTLTIKLEQDRGSHC | |
| Soluble | 23-555 | QPDPPCGGRLNSKDAGYITSPGYPQDYPSHQNCEWIVYAPEPN | 798 |
| neuropilin | | QKIVLNFNPHFEIEKHDCKYDFIEIRDGDSESADLLGKHCGNI | |
| 2 2 | | APPTIISSGSMLYIKFTSDYARQGAGFSLRYEIFKTGSEDCSK | |
| | | NFTSPNGTIESPGFPEKYPHNLDCTFTILAKPKMEIILQFLIF | |
| | | DLEHDPLQVGEGDCKYDWLDIWDGIPHVGPLIGKYCGTKTPSE | |
| | | LRSSTGILSLTFHTDMAVAKDGFSARYYLVHQEPLENFQCNVP | |
| | | LGMESGRIANEQISASSTYSDGRWTPQQSRLHGDDNGWTPNLD | |
| | | SNKEYLQVDLRFLTMLTAIATQGAISRETQNGYYVKSYKLEVS | |
| | | TNGEDWMVYRHGKNHKVFQANNDATEVVLNKLHAPLLTRFVRI | |
| | | RPQTWHSGIALRLELFGCRVTDAPCSNMLGMLSGLIADSQISA | |
| | | SSTQEYLWSPSAARLVSSRSGWFPRIPQAQPGEEWLQVDLGTP | |
| | | KTVKGVIIQGARGGDSITAVEARAFVRKFKVSYSLNGKDWEYI | |
| | | QDPRTQQPKVGCSWRPL | |
| Neuropilin | 28-141 | CGGRLNSKDAGYITSPGYPQDYPSHQNCEWIVYAPEPNQKIVL | 799 |
| 2 A1 | | NFNPHFEIEKHDCKYDFIEIRDGDSESADLLGKHCGNIAPPTI | |
| domain | | ISSGSMLYIKFTSDYARQGAGFSLRYEI | |
| Neuropilin | 149-265 | CSKNFTSPNGTIESPGFPEKYPHNLDCTFTILAKPKMEIILQF | 800 |
| 2 A2 | | LIFDLEHDPLQVGEGDCKYDWLDIWDGIPHVGPLIGKYCGTKT | |
| domain | | PSELRSSTGILSLTFHTDMAVAKDGFSARYY | |
| Neuropilin | 280-426 | PLGMESGRIANEQISASSTYSDGRWTPQQSRLHGDDNGWTPNL | 801 |
| 2 B1 | | DSNKEYLQVDLRFLTMLTAIATQGAISRETQNGYYVKSYKLEV | |
| domain | | STNGEDWMVYRHGKNHKVFQANNDATEVVLNKLHAPLLTRFVR | |
| | | IRPQTWHSGIALRLELFG | |
| Neuropilin | 438-591 | LGMLSGLIADSQISASSTQEYLWSPSAARLVSSRSGWFPRIPQ | 802 |
| 2 B2 | | AQPGEEWLQVDLGTPKTVKGVIIQGARGGDSITAVEARAFVRK | |
| domain | | FKVSYSLNGKDWEYIQDPRTQQPKLFEGNMHYDTPDIRRFDPI | |
| | | PAQYVRVYPERWSPAGIGMRLEVLG | |
| Neuropilin | 641-794 | PSGFNCNFDFLEEPCGWMYDHAKWLRTTWASSSSPNDRTFPDD | 803 |
| 2C domain | | RNFLRLQSDSQREGQYARLISPPVHLPRSPVCMEFQYQATGGR | |
| | | GVALQVVREASQESKLLWVIREDQGGEWKHGRIILPSYDMEYQ | |
| | | IVFEGVIGKGRSGEIAIDDIRISTD | |
| Neuropilin | 23-595 | QPDPPCGGRLNSKDAGYITSPGYPQDYPSHQNCEWIVYAPEPN | 804 |
| 2 A1A2B1B2 | | QKIVLNFNPHFEIEKHDCKYDFIEIRDGDSESADLLGKHCGNI | |
| | | APPTIISSGSMLYIKFTSDYARQGAGFSLRYEIFKTGSEDCSK | |
| | | NFTSPNGTIESPGFPEKYPHNLDCTFTILAKPKMEIILQFLIF | |
| | | DLEHDPLQVGEGDCKYDWLDIWDGIPHVGPLIGKYCGTKTPSE | |
| | | LRSSTGILSLTFHTDMAVAKDGFSARYYLVHQEPLENFQCNVP | |
| | | LGMESGRIANEQISASSTYSDGRWTPQQSRLHGDDNGWTPNLD | |
| | | SNKEYLQVDLRFLTMLTAIATQGAISRETQNGYYVKSYKLEVS | |
| | | TNGEDWMVYRHGKNHKVFQANNDATEVVLNKLHAPLLTREVRI | |
| | | RPQTWHSGIALRLELFGCRVTDAPCSNMLGMLSGLIADSQISA | |
| | | SSTQEYLWSPSAARLVSSRSGWFPRIPQAQPGEEWLQVDLGTP | |
| | | KTVKGVIIQGARGGDSITAVEARAFVRKFKVSYSLNGKDWEYI | |
| | | QDPRTQQPKLFEGNMHYDTPDIRRFDPIPAQYVRVYPERWSPA | |
| | | GIGMRLEVLGCDWT | |
| Neuropilin | 145-595 | GSEDCSKNFTSPNGTIESPGFPEKYPHNLDCTFTILAKPKMEI | 805 |
| 2 A2B1B2 | | ILQFLIFDLEHDPLQVGEGDCKYDWLDIWDGIPHVGPLIGKYC | |
| | | GTKTPSELRSSTGILSLTFHTDMAVAKDGFSARYYLVHQEPLE | |
| | | NFQCNVPLGMESGRIANEQISASSTYSDGRWTPQQSRLHGDDN | |
| | | GWTPNLDSNKEYLQVDLRFLTMLTAIATQGAISRETQNGYYVK | |
| | | SYKLEVSTNGEDWMVYRHGKNHKVFQANNDATEVVLNKLHAPL | |
| | | LTRFVRIRPQTWHSGIALRLELFGCRVTDAPCSNMLGMLSGLI | |
| | | ADSQISASSTQEYLWSPSAARLVSSRSGWFPRIPQAQPGEEWL | |
| | | QVDLGTPKTVKGVIIQGARGGDSITAVEARAFVRKFKVSYSLN | |
| | | GKDWEYIQDPRTQQPKLFEGNMHYDTPDIRRFDPIPAQYVRVY | |
| | | PERWSPAGIGMRLEVLGCDWT | |
| Neuropilin | 276-595 | QCNVPLGMESGRIANEQISASSTYSDGRWTPQQSRLHGDDNGW | 806 |
| 2 B1B2 | | TPNLDSNKEYLQVDLRFLTMLTAIATQGAISRETQNGYYVKSY | |
| | | KLEVSTNGEDWMVYRHGKNHKVFQANNDATEVVLNKLHAPLLT | |
| | | RFVRIRPQTWHSGIALRLELFGCRVTDAPCSNMLGMLSGLIAD | |
| | | SQISASSTQEYLWSPSAARLVSSRSGWFPRIPQAQPGEEWLQV | |
| | | DLGTPKTVKGVIIQGARGGDSITAVEARAFVRKFKVSYSLNGK | |
| | | DWEYIQDPRTQQPKLFEGNMHYDTPDIRRFDPIPAQYVRVYPE | |
| | | RWSPAGIGMRLEVLGCDWT | |
| Neuropilin | 23-855 | QPDPPCGGRLNSKDAGYITSPGYPQDYPSHQNCEWIVYAPEPN | 807 |
| 2 v2-Fc | | QKIVLNFNPHFEIEKHDCKYDFIEIRDGDSESADLLGKHCGNI | |
| fusion | | APPTIISSGSMLYIKFTSDYARQGAGFSLRYEIFKTGSEDCSK | |
| protein | | NFTSPNGTIESPGFPEKYPHNLDCTFTILAKPKMEIILQFLIF | |
| | | DLEHDPLQVGEGDCKYDWLDIWDGIPHVGPLIGKYCGTKTPSE | |
| | | LRSSTGILSLTFHTDMAVAKDGFSARYYLVHQEPLENFQCNVP | |
| | | LGMESGRIANEQISASSTYSDGRWTPQQSRLHGDDNGWTPNLD | |
| | | SNKEYLQVDLRFLTMLTAIATQGAISRETQNGYYVKSYKLEVS | |
| | | TNGEDWMVYRHGKNHKVFQANNDATEVVLNKLHAPLLTRFVRI | |
| | | RPQTWHSGIALRLELFGCRVTDAPCSNMLGMLSGLIADSQISA | |
| | | SSTQEYLWSPSAARLVSSRSGWFPRIPQAQPGEEWLQVDLGTP | |
| | | KTVKGVIIQGARGGDSITAVEARAFVRKFKVSYSLNGKDWEYI | |
| | | QDPRTQQPKLFEGNMHYDTPDIRRFDPIPAQYVRVYPERWSPA | |
| | | GIGMRLEVLGCDWTDSKPTVETLGPTVKSEETTTPYPTEEEAT | |
| | | ECGENCSFEDDKDLQLPSGFNCNFDFLEEPCGWMYDHAKWLRT | |
| | | TWASSSSPNDRTFPDDRNFLRLQSDSQREGQYARLISPPVHLP | |
| | | RSPVCMEFQYQATGGRGVALQVVREASQESKLLWVIREDQGGE | |
| | | WKHGRIILPSYDMEYQIVFEGVIGKGRSGEIAIDDIRISTDVP | |
| | | LENCMEPISAFAVDIPEIHEREGYEDEIDDEYEVDWSNSSSAT | |
| | | SGSGAPSTDKEKSWLYDKTHTCPPCPAPELLGGPSVFLFPPKP | |
| | | KDTLMISRTPEVTCVVVDVSHEDPEVKFNWYVDGVEVHNAKTK | |
| | | PREEQYNSTYRVVSVLTVLHQDWLNGKEYKCKVSNKALPAPIE | |
| | | KTISKAKGQPREPQVYTLPPSRDELTKNQVSLTCLVKGFYPSD | |
| | | IAVEWESNGQPENNYKTTPPVLDSDGSFFLYSKLTVDKSRWQQ | |
| | | GNVFSCSVMHEALHNHYTQKSLSLSPGK | |
| Neuropilin | 145-595 | GSEDCSKNFTSPNGTIESPGFPEKYPHNLDCTFTILAKPKMEI | 808 |
| A2B1B2-Fc | | ILQFLIFDLEHDPLQVGEGDCKYDWLDIWDGIPHVGPLIGKYC | |
| | | GTKTPSELRSSTGILSLTFHTDMAVAKDGFSARYYLVHQEPLE | |
| | | NFQCNVPLGMESGRIANEQISASSTYSDGRWTPQQSRLHGDDN | |
| | | GWTPNLDSNKEYLQVDLRFLTMLTAIATQGAISRETQNGYYVK | |
| | | SYKLEVSTNGEDWMVYRHGKNHKVFQANNDATEVVLNKLHAPL | |
| | | LTRFVRIRPQTWHSGIALRLELFGCRVTDAPCSNMLGMLSGLI | |
| | | ADSQISASSTQEYLWSPSAARLVSSRSGWFPRIPQAQPGEEWL | |
| | | QVDLGTPKTVKGVIIQGARGGDSITAVEARAFVRKFKVSYSLN | |
| | | GKDWEYIQDPRTQQPKLFEGNMHYDTPDIRRFDPIPAQYVRVY | |
| | | PERWSPAGIGMRLEVLGCDWTDKTHTCPPCPAPELLGGPSVFL | |
| | | FPPKPKDTLMISRTPEVTCVVVDVSHEDPEVKFNWYVDGVEVH | |
| | | NAKTKPREEQYNSTYRVVSVLTVLHQDWLNGKEYKCKVSNKAL | |
| | | PAPIEKTISKAKGQPREPQVYTLPPSRDELTKNQVSLTCLVKG | |
| | | FYPSDIAVEWESNGQPENNYKTTPPVLDSDGSFFLYSKLTVDK | |
| | | SRWQQGNVFSCSVMHEALHNHYTQKSLSLSPGK | |

In some instances, an antibody or antigen-binding fragment thereof specifically binds to at least one epitope within a region or domain of a human HRS polypeptide (selected, for example, from Table H1) that binds to or interacts with at least one NP2 polypeptide selected from Table N2. In some instances, an antibody or antigen-binding fragment thereof specifically binds to at least one epitope within a region of a human HRS polypeptide that binds to or interacts with at least one neuropilin domain. In some embodiments, the neuropilin domain is selected from one or more of the Neuropilin A1 domain, Neuropilin A2 domain, neuropilin B1 domain, neuropilin B2 domain, neuropilin C domain, neuropilin A1A2 combined domain, neuropilin B1B2 combined domain, neuropilin A2B1 combined domain, neuropilin A2B1B2 combined domain, neuropilin A2B1B2C combined domain, neuropilin A1A2B1 combined domain, neuropilin A1A2B1B2 combined domain, and the neuropilin A1A2B1B2C combined domain.

In certain embodiments, an antibody or antigen-binding fragment thereof is a “blocking antibody”, which fully or substantially inhibits the binding between a human HRS polypeptide (selected, for example, from Table H1) and an NP2 polypeptide (selected, for example, from Table N2). In some embodiments, a “blocking antibody” inhibits about or at least about 80-100% (e.g., 80, 85, 90, 95, or 100%) of the theoretical maximal binding between the HRS polypeptide and the NP2 polypeptide after pre-incubation of the “blocking antibody” with the HRS polypeptide in a substantially of fully stoichiometrically equivalent amount. As used herein, a “stoichiometrically equivalent amount” refers to a situation where the number of moles of one substance (e.g., HRS antibody) is equivalent or substantially equivalent to the number of moles at least one other substance (e.g., HRS polypeptide) in a given equation or reaction. In some embodiments, a “blocking antibody” specifically binds to at least one epitope within the N-terminal region of the human HRS polypeptide, for example, within about residues 1-100 of SEQ ID NO: 1, or within about the WHEP domain (˜residues 1-60 or ˜residues 3-43). In certain embodiments, a “blocking antibody” specifically binds to at least one epitope within the aminoacylation domain (˜residues 54-398 or ˜residues 61-398) of the human HRS polypeptide. In particular embodiments, a “blocking antibody” specifically binds to at least one epitope within the anticodon domain (˜residues 399-509 including a core domain of ˜residues 406-501) of the human HRS polypeptide. In some embodiments, a “blocking antibody” specifically binds to a HRS splice variant of Table H1. In certain embodiments, a “blocking antibody” specifically binds to a HRS splice variant selected from SV9 (HRS(1-60)), SV11 (HRS(1-60)+(399-509)) and SV14 (HRS(1-100)+(399-509)). In certain embodiments, a “blocking antibody” selectively binds only to a monomeric form of the HRS polypeptide, and does not substantially bind to a dimeric or multimeric form of the HRS polypeptide.

In certain embodiments, an antibody or antigen-binding fragment thereof is a “partial-blocking antibody”, which at least partially but not fully inhibits the binding between a human HRS polypeptide (selected, for example, from Table H1) and an NP2 polypeptide (selected, for example, from Table N2). In some embodiments, a “partial-blocking antibody” inhibits about or at least about 20-80% (e.g., 20, 25, 30, 35, 40, 45, 50, 55, 60, 65, 70, 75, or 80%) of the theoretical maximal binding between the HRS polypeptide and the NP2 polypeptide after pre-incubation of the “partial-blocking antibody” with the HRS polypeptide in a stoichiometric amount. In some embodiments, a “partial-blocking antibody” specifically binds to at least one epitope within the N-terminal region of the human HRS polypeptide, for example, within about residues 1-100 of SEQ ID NO: 1, or within about the WHEP domain (˜residues 1-60 or ˜residues 3-43). In certain embodiments, a “partial-blocking antibody” specifically binds to at least one epitope within the aminoacylation domain (˜residues 54-398 or ˜residues 61-398) of the human HRS polypeptide. In particular embodiments, a “partial-blocking antibody” specifically binds to at least one epitope within the anticodon domain (˜residues 399-509 including a core domain of ˜residues 406-501) of the human HRS polypeptide. In some embodiments, a “partial-blocking antibody” specifically binds to a HRS splice variant of Table H1. In certain embodiments, a “partial-blocking antibody” specifically binds to a HRS splice variant selected from SV9 (HRS(1-60)), SV11 (HRS(1-60)+(399-509)) and SV14 (HRS(1-100)+(399-509)). In certain embodiments, a “partial-blocking antibody” selectively binds only to a monomeric form of the HRS polypeptide, and does not substantially bind to a dimeric or multimeric form of the HRS polypeptide.

In certain embodiments, an antibody or antigen-binding fragment thereof is a “non-blocking antibody”, which does not substantially inhibit the binding between a human HRS polypeptide (selected, for example, from Table H1) and an NP2 polypeptide (selected, for example, from Table N2). In some embodiments, a “non-blocking antibody” inhibits about or less than about 10% (e.g., 2, 4, 6, 8, or 10%) of the theoretical maximal binding between the HRS polypeptide and the NP2 polypeptide after pre-incubation of the “non-blocking antibody” with the HRS polypeptide in a stoichiometric amount. In some embodiments, a “non-blocking antibody” specifically binds to at least one epitope within the N-terminal region of the human HRS polypeptide, for example, within about residues 1-100 of SEQ ID NO: 1, or within about the WHEP domain (˜residues 1-60 or ˜residues 3-43). In certain embodiments, a “non-blocking antibody” specifically binds to at least one epitope within the aminoacylation domain (˜residues 54-398 or ˜residues 61-398) of the human HRS polypeptide. In particular embodiments, a “non-blocking antibody” specifically binds to at least one epitope within the anticodon domain (˜residues 399-509 including a core domain of ˜residues 406-501) of the human HRS polypeptide. In some embodiments, a “non-blocking antibody” specifically binds to a HRS splice variant of Table H1. In certain embodiments, a “non-blocking antibody” specifically binds to a HRS splice variant selected from SV9 (HRS(1-60)), SV11 (HRS(1-60)+(399-509)) and SV14 (HRS(1-100)+(399-509)). In certain embodiments, a “non-blocking antibody” selectively binds only to a monomeric form of the HRS polypeptide, and does not substantially bind to a dimeric or multimeric form of the HRS polypeptide.

Merely for illustrative purposes, the binding interactions between an HRS polypeptide and an NP2 polypeptide can be detected and quantified using a variety of routine methods, including biacore assays (for example, with appropriately tagged soluble reagents, bound to a sensor chip), FACS analyses with cells expressing a NP2 polypeptide on the cell surface (either native, or recombinant), immunoassays, fluorescence staining assays, ELISA assays, and microcalorimetry approaches such as ITC (Isothermal Titration Calorimetry).

In some embodiments, an antibody or antigen-binding fragment thereof cross-reacts with HRS polypeptide homologs from other mammals. For instance, in certain embodiments, an antibody or antigen-binding fragment thereof specifically binds to an HRS polypeptide that comprises, consists, or consists essentially of an amino acid sequence in Table H2 (e.g., SEQ ID NO: ______) or an active variant or fragment thereof.

TABLE H2
Exemplary Mammalian Homologs of Human HRS

| | | SEQ ID |
|---|---|---|
| Species | Amino acid Sequence | NO: |
| Mus musculus | MADRAALEELVRLQGAHVRGLKEQKASAEQIEEEVIKLLKLKAQLGQDEG | 809 |
| | KQKFVLKTPKGTRDYSPRQMAVREKVFDVIIRCFKRHGAEVIDTPVFELK | |
| | ETLTGKYGEDSKLIYDLKDQGGELLSLRYDLTVPFARYLAMNKLTNIKRY | |
| | HIAKVYRRDNPAMTRGRYREFYQCDFDIAGQFDPMIPDAECLKIMCEILS | |
| | SLQIGNELVKVNDRRILDGMFAVCGVPDSKERTICSSVDKLDKVSWEEVK | |
| | NEMVGEKGLAPEVADRIGDYVQQHGGVSLVEQLLQDPKLSQNKQAVEGLG | |
| | DLKLLFEYLILFGIDDKISFDLSLARGLDYYTGVIYEAVLLQMPTQAGEE | |
| | PLGVGSIAAGGRYDGLVGMFDPKGRKVPCVGLSIGVERIFSIVEQRLEAS | |
| | EEKVRTTETQVLVASAQKKLLEERLKLVSELWDAGIKAELLYKKNPKLLN | |
| | QLQYWEEAGIPLVAIIGEQELRDGVIKLRSVASREEVDVRREDLVEEIRR | |
| | RTNQPLSTC | |
| Canis lupus | MAERAALEELVRLQGERVRGLKQQKASAEQIEEEVAKLLKLKAQLGPDEG | 810 |
| familiaris | KQKFVLKTPKGTRDYSPRQMAVREKVFDVIISCFKRHGAEVIDTPVFELK | |
| | ETLTGKYGEDSKLIYDLKDQGGELLSLRYDLTVPFARYLAMNKLTNIKRY | |
| | HIAKVYRRDNPAMTRGRYREFYQCDFDIAGQFDPMIPDAECLEIMCEILR | |
| | SLQIGDFLVKVNDRRILDGMFAICGVPDSKERTICSSVDKLDKVSWEEVK | |
| | NEMVGEKGLAPEVADHIGDYVQQHGGISLVEQLLQDPELSQNKQALEGLG | |
| | DLKLLFEYLTLFGIADKISFDLSLARGLDYYTGVIYEAVLLQTPVQAGEE | |
| | PLGVGSVAAGGRYDGLVGMFDPKGRKVPCVGLSIGVERIFSIVEQRLEAT | |
| | EEKVRTTETQVLVASAQKKLLEERLKLVSELWNAGIKAELLYKKNPKLLN | |
| | QLQYCEEAGIPLVAIIGEQELKDGVIKLRSVASREEVDVPREDLVEEIKR | |
| | RTSQPFCIC | |
| Bos taurus | MADRAALEDLVRVQGERVRGLKQQKASAEQIEEEVAKLLKLKAQLGPDEG | 811 |
| | KPKFVLKTPKGTRDYSPRQMAVREKVFDVIISCFKRHGAEVIDTPVFELK | |
| | ETLTGKYGEDSKLIYDLKDQGGELLSLRYDLTVPFARYLAMNKLTNIKRY | |
| | HIAKVYRRDNPAMTRGRYREFYQCDFDIAGQFDPMLPDAECLKIMCEILS | |
| | SLQIGDFLVKVNDRRILDGMFAICGVPDSKERTICSSVDKLDKVSWEEVK | |
| | NEMVGEKGLAPEVADRIGDYVQQHGGVSLVEQLLQDPKLSQNKQALEGLG | |
| | DLKLLFEYLTLFGIADKISFDLSLARGLDYYTGVIYEAVLLQPPARAGEE | |
| | PLGVGSVAAGGRYDGLVGMFDPKGRKVPCVGLSIGVERIFSIVEQRLEAL | |
| | EEKVRTTETQVLVASAQKKLLEERLKLISELWDAGIKAELLYKKNPKLLN | |
| | QLQYCEETGIPLVAIIGEQELKDGVIKLRSVASREEVDVR | |
| | REDLVEEIKR RTSQPLCIC | |
| Rattus | MADRAALEELVRLQGAHVRGLKEQKASAEQIEEEVTKLLKLKAQLGHDEG | 812 |
| norvegicus | KQKFVLKTPKGIRDYSPRQMAVREKVFDVIIRCFKRHGAEVIDTPVFELK | |
| | ETLTGKYGEDSKLIYDLKDQGGELLSLRYDLTVPFARYLAMNKLTNIKRY | |
| | HIAKVYRRDNPAMTRGRYREFYQCDFDIAGQFDPMIPDAECLKIMCEILS | |
| | SLQIGNFQVKVNDRRILDGMFAVCGVPDSKERTICSSVDKLDKVSWEEVK | |
| | NEMVGEKGLAPEVADRIGDYVQQHGGVSLVEQLLQDPKLSQNKQAVEGLG | |
| | DLKLLFEYLTLFGIDDKISFDLSLARGLDYYTGVIYEAVLLQMPTQAGEE | |
| | PLGVGSIAAGGRYDGLVGMFDPKGRKVPCVGLSIGVERIFSIVEQKLEAS | |
| | EEKVRTTETQVLVASAQKKLLEERLKLISELWDAGIKAELLYKKNPKLLN | |
| | QLQYCEEAGI PLVAIIGEQE LKDGVIKLRSVTSREEVDVR | |
| | REDLVEEIRR RTSQPLSM | |
| Gallus | MADEAAVRQQAEVVRRLKQDKAEPDEIAKEVAKLLEMKAHLGGDEGKHKF | 813 |
| | VLKTPKGTRDYGPKQMAIRERVFSAIIACFKRHGAEVIDTPVFELKETLT | |
| | GKYGEDSKLIYDLKDQGGELLSLRYDLTVPFARYLAMNKITNIKRYHIAK | |
| | VYRRDNPAMTRGRYREFYQCDFDIAGQFDPMIPDAECLKIVQEILSDLQL | |
| | GDFLIKVNDRRILDGMFAVCGVPDSKFRTICSSVDKLDKMPWEEVRNEMV | |
| | GEKGLSPEAADRIGEYVQLHGGMDLIEQLLQDPKLSQNKLVKEGLGDMKL | |
| | LFEYLTLFGITGKISFDLSLARGLDYYTGVIYEAVLLQQNDHGEESVSVG | |
| | SVAGGGRYDGLVGMFDPKGR | |
| | KVPCVGISIGIERIFSILEQRVEASEEKIR | |
| | TTETQVLVASAQKKLLEERLKLISELWDAGIKAEVLYKKNPKLLNQLQYC | |
| | EDTGIPLVAIVGEQELKDGVVKLRVVATGEEVNIRRESLVEEIRRRTNQL | |
| Danio rerio | MAALGLVSMRLCAGLMGRRSAVRLHSLRVCSGMTISQIDEEVARLLQLKA | 814 |
| | QLGGDEGKHVFVLKTAKGTRDYNPKQMAIREKVFNIIINCFKRHGAETID | |
| | SPVFELKETLTGKYGEDSKLIYDLKDQGGELLSLRYDLTVPFARYLAMNK | |
| | ITNIKRYHIAKVYRRDNPAMTRGRYREFYQCDFDIAGQYDAMIPDAECLK | |
| | LVYEILSELDLGDFRIKVNDRRILDGMFAICGVPDEKFRTICSTVDKLDK | |
| | LAWEEVKKEMVNEKGLSEEVADRIRDYVSMQGGKDLAERLLQDPKLSQSK | |
| | QACAGITDMKLLFSYLELFQITDKVVFDLSLARGLDYYTGVIYEAILTQA | |
| | NPAPASTPAEQNGAEDAGVSVGSVAGGGRYDGLVGMFDPKAGKCPVWGSA | |
| | LALRGSSPSWSRRQSCLQRRCAPLKLKCLWLQHRRTF | |
| Macaca | MAERAALEELVKLQGERVRGLKQQQASAELIEEEVGKLLKLKAQLGPDES | 815 |
| fascicularis | KQKFVLKTPKGIRDYSPRQMAVREKVFDVIIRCFKRHGAEVIDTPVFELK | |
| | DFDIAGNFDPMIPDAECLKIMCEILSSLQIGDFLVKVNDRRILDGMFAIC | |
| | GVSDSKFRTICSSVDKLDKVSWEEVKNEMVGEKGLAPEVADRIGDYVQQH | |
| | GGVSLVEQLLQDPKLSQNKQALEGLGDLKLLFEYLTLFGIDDKISFDLSL | |
| | ARGLDYYTGVIYEAVLLQTPAQAGEEPLGVGSVAAGGRYDGLVGMFDPKG | |
| | RKVPCVGLSIGVERIFSIVEQRLEALEEKVRTTETQVLVASAQKKLLEER | |
| | LKLVSELWDAGIKAELLYKKNPKLLNQLQYCEEAGIPLVAIIGEQELKDG | |
| | VIKLRSVISREEVNVRREDLVEEIKRRTGQLLRIC | |
| Macaca | MAERAALEELVKLQGERVRGLKQQKASAELIEEEVGKLLKLKAQLGPDES | 816 |
| mulatta | KQKFVLKTPKGTRDYSPRQMAVREKVFDVIIRCFKRHGAEVIDTPVFELK | |
| | ETLMGKYGEDSKLIYDLKDQGGELLSLRYDLTVPFARYLAMNKLTNIKRY | |
| | HIAKVYRRDNPAMTRGRYREFYQCDFDIAGNFDPMIPDAECLKIMCEILS | |
| | SLQIGDFLVKVNDRRILDGMFAICGVSDSKERTICSSVDKLDKVSWEEVK | |
| | NEMVGEKGLAPEVADRIGDYVQQHGGVSLVEQLLQDPKLSQNKQALEGLG | |
| | DLKLLFEYLTLFGIDDKISFDLSLARGLDYYTGVIYEAVLLQTPAQAGEE | |
| | PLGVGSVAAGGRYDGLVGMFDPKGRKVPCVGLSIGVERIFSIVEQRLEAL | |
| | EEKVRTTETQVLVASAQKKLLEERLKLVSELWDAGIKAELLYKKNPKLLN | |
| | QLQYCEEAGIPLVAIIGEQELKDGVIKLRSVTSREEVNVRREDLVEEIKR | |
| | RTGQPLRIC | |

In specific embodiments, an antibody or antigen-binding fragment thereof has an affinity (Kd) for a human HRS polypeptide or epitope or complex (e.g., human HRS:human NP2 complex) described herein (to which it specifically binds) of about, at least about, or less than about 10 pM to about 500 pM to about 1 nM, or about 10, 20, 30, 40, 50, 60, 70, 80, 90, 100, 110, 120, 130, 140, 150, 160, 170, 180, 190, 200, 300, 400, 500, 600, 700, 800, 900 pM or 1 nM, including all integers and ranges in between, for example, about 10 pM to about 500 pM, about 10 pM to about 400 pM, about 10 pM to about 300 pM, about 10 pM to about 200 pM, about 10 pM to about 100 pM, about 10 pM to about 50 pM, or about 20 pM to about 500 pM, about 20 pM to about 400 pM, about 20 pM to about 300 pM, about 20 pM to about 200 pM, about 20 pM to about 100 pM, about 20 pM to about 50 pM, or about 30 pM to about 500 pM, about 30 pM to about 400 pM, about 30 pM to about 300 pM, about 30 pM to about 200 pM, about 30 pM to about 100 pM, about 30 pM to about 50 pM, or about 20 pM to about 200 pM, about 30 pM to about 300 pM, about 40 pM to about 400 pM, about 50 pM to about 500 pM, about 60 pM to about 600 pM, about 70 pM to about 700 pM, about 80 pM to about 800 pM, about 90 pM to about 900 pM, or about 100 pM to about 1 nM.

In certain embodiments, an antibody or antigen-binding fragment thereof is cross reactive between HRS polypeptides from different species, for example, selected from Table H2. In some embodiments, an antibody or antigen-binding fragment thereof has an affinity (Kd) for a non-human HRS polypeptide which is within about 1 log of the affinity for the same epitope region of the human HRS polypeptide. In specific embodiments, an antibody or antigen-binding fragment thereof has an affinity (Kd) for a cynomolgus monkey (Macaca fascicularis) HRS polypeptide, or a Rhesus monkey (Macaca mulatta) HRS polypeptide, which is within about 1 log of the affinity for the same epitope region of the human HRS polypeptide. In specific embodiments, an antibody or antigen-binding fragment thereof has an affinity (Kd) for both a human HRS polypeptide described herein (to which it specifically binds) and the corresponding region of a cynomolgus or Rhesus monkey HRS polypeptide, where an antibody affinity for both proteins falls within the range of about 20 pM to about 200 pM, or about 30 pM to about 300 pM, or about 40 pM to about 400 pM, or about 50 pM to about 500 pM, or about 60 pM to about 600 pM, or about 70 pM to about 700 pM, or about 80 pM to about 800 pM, or about 90 pM to about 900 pM, and/or about 100 pM to about 1 nM, including all integers and ranges in between. In some embodiments, an antibody or antigen-binding fragment thereof has an affinity (Kd) for a rodent (e.g., mouse or rat) HRS polypeptide which is within about 1 log of the affinity for the same epitope region of the human HRS polypeptide.

In certain embodiments, an antibody or antigen-binding fragment thereof is characterized by or comprises a heavy chain variable region (V_{H}) sequence that comprises complementary determining region V_{H}CDR1, V_{H}CDR2, and V_{H}CDR3 sequences, and a light chain variable region (V_{L}) sequence that comprises complementary determining region V_{L}CDR1, V_{L}CDR2, and V_{L}CDR3 sequences. Exemplary V_{H}, V_{H}CDR1, V_{H}CDR2, V_{H}CDR3, VL, V_{L}CDR1, V_{L}CDR2, and V_{L}CDR3 sequences are provided in Table A1 and Table A2 below. Table A3 provides the amino acids for the CDR “consensus” sequences of SEQ ID NOs:396-413.

TABLE A1
Exemplary Polypeptide Sequences

| | | SEQ |
|---|---|---|
| | | ID |
| Description | Sequence | NO: |
| V_{H}CDR1 | GYTFTDYCIG | 12 |
| Ab/clone | | |
| KL31-418 | | |
| V_{H}CDR2 | DICPGDTYTNDNEKFKD | 13 |
| Ab/clone | | |
| KL31-418 | | |
| V_{H}CDR3 | GEEQLGLRNAMDY | 14 |
| Ab/clone | | |
| KL31-418 | | |
| V_{L}CDR1 | QSQSVSTSTYNYMH | 15 |
| Ab/clone | | |
| KL31-418 | | |
| V_{L}CDR2 | YASNLES | 16 |
| Ab/clone | | |
| KL31-418 | | |
| V_{L}CDR3 | GHSYEIPWT | 17 |
| Ab/clone | | |
| KL31-418 | | |
| V_{H}CDR1 | GFTFSDYYMT | 18 |
| Ab/clone | | |
| AB04-168 | | |
| V_{H}CDR2 | YISGSFRYTNYADKVKG | 19 |
| Ab/clone | | |
| AB04-168 | | |
| V_{H}CDR3 | YVYQVVAIGDL | 20 |
| Ab/clone | | |
| AB04-168 | | |
| V_{L}CDR1 | RASQGISSWLA | 21 |
| Ab/clone | | |
| AB04-168 | | |
| V_{L}CDR2 | AASSLQS | 22 |
| Ab/clone | | |
| AB04-168 | | |
| V_{L}CDR3 | QQAESFPYT | 23 |
| Ab/clone | | |
| AB04-168 | | |
| V_{H}CDR1 | GFTFSDYYMS | 24 |
| Ab/clone | | |
| AB13-112 | | |
| V_{H}CDR2 | YISDKSRYTKYTDKVRG | 25 |
| Ab/clone | | |
| AB13-112 | | |
| V_{H}CDR3 | YLYQVIAIADA | 26 |
| Ab/clone | | |
| AB13-112 | | |
| V_{L}CDR1 | RASQGISSWLA | 27 |
| Ab/clone | | |
| AB13-112 | | |
| V_{L}CDR2 | VASNLES | 28 |
| Ab/clone | | |
| AB13-112 | | |
| V_{L}CDR3 | QQAESFPYT | 29 |
| Ab/clone | | |
| AB13-112 | | |
| V_{H}CDR1 | GYTFX₃₀X₃₁X₃₂CX₃₃X₃₄ | 396 |
| KL31 series | | |
| Consensus | | |
| V_{H}CDR2 | X₃₅X₃₆CX₃₇X₃₈X₃₉X₄₀X₄₁X₄₂X₄₃D | 397 |
| KL31 series | X₄₄EKFKX₄₅ | |
| Consensus | | |
| V_{H}CDR3 | X₄₆X₄₇X₄₈X₄₉X₅₀X₅₁L | 398 |
| KL31 series | X₅₂X₅₃X₅₄X₅₅X₅₆X₅₇ | |
| Consensus | | |
| V_{L}CDR1 | QSQSVSTSTYNYMH | 399 |
| KL31 series | | |
| Consensus | | |
| V_{L}CDR2 | YASNLES | 400 |
| KL31 series | | |
| Consensus | | |
| V_{L}CDR3 | X₅₈X₅₉X₆₀X₆₁X₆₂X₆₃X₆₄X₆₅X₆₆ | 401 |
| KL31 series | | |
| Consensus | | |
| V_{H}CDR1 | GFTFX₁X₂YYX₃X₄ | 402 |
| AB04 series | | |
| Consensus | | |
| V_{H}CDR2 | YX₅SGX₆X₇X₈YX₉X₁₀X₁₁AX₁₂X₁₃VKG | 403 |
| AB04 series | | |
| Consensus | | |
| V_{H}CDR3 | YX₁₄YQX₁₅X₁₆X₁₇X₁₈X₁₉X₂₀X₂₁ | 404 |
| AB04 series | | |
| Consensus | | |
| V_{L}CDR1 | RASQGISSWLA | 405 |
| AB04 series | | |
| Consensus | | |
| V_{L}CDR2 | AASSLQS | 406 |
| AB04 series | | |
| Consensus | | |
| V_{L}CDR3 | X₂₂X₂₃X₂₄X₂₅X₂₆FX₂₇X₂₈X₂₉ | 407 |
| AB04 series | | |
| Consensus | | |
| V_{H}CDR1 | GFTESDYYMX₆₇ | 408 |
| AB13 series | | |
| Consensus | | |
| V_{H}CDR2 | YISX₆₈X₆₉X₇₀X₇₁YTX₇₂YX₇₃X₇₄X₇₅VRG | 409 |
| AB13 series | | |
| Consensus | | |
| V_{H}CDR3 | X₇₆X₇₇X₇₈X₇₉VX₈₀X₈₁X₈₂X₈₃X₈₄X₈₅ | 410 |
| AB13 series | | |
| Consensus | | |
| V_{L}CDR1 | RASQGISSWLA | 411 |
| AB13 series | | |
| Consensus | | |
| V_{L}CDR2 | VASNLES | 412 |
| AB13 series | | |
| Consensus | | |
| V_{L}CDR3 | QQAX₈₆SFPYT | 413 |
| AB13 series | | |
| Consensus | | |
| V_{H}CDR1 | GFTFSDYYMT | 36 |
| Ab/clone | | |
| AB04-121 | | |
| V_{H}CDR2 | YISGSNAYTDYADSVKG | 37 |
| Ab/clone | | |
| AB04-121 | | |
| V_{H}CDR3 | YVYQVVAIGDY | 38 |
| Ab/clone | | |
| AB04-121 | | |
| V_{L}CDR1 | RASQGISSWLA | 39 |
| Ab/clone | | |
| AB04-121 | | |
| V_{L}CDR2 | AASSLQS | 40 |
| Ab/clone | | |
| AB04-121 | | |
| V_{L}CDR3 | QQAKSFPYT | 41 |
| Ab/clone | | |
| AB04-121 | | |
| V_{H}CDR1 | GFTFSDYYMT | 42 |
| Ab/clone | | |
| AB04-174 | | |
| V_{H}CDR2 | YISGSNAYTDYADSVKG | 43 |
| Ab/clone | | |
| AB04-174 | | |
| V_{H}CDR3 | YVYQTVAIGDL | 44 |
| Ab/clone | | |
| AB04-174 | | |
| V_{L}CDR1 | RASQGISSWLA | 45 |
| Ab/clone | | |
| AB04-174 | | |
| V_{L}CDR2 | AASSLQS | 46 |
| Ab/clone | | |
| AB04-174 | | |
| V_{L}CDR3 | QQAKSFPYT | 47 |
| Ab/clone | | |
| AB04-174 | | |
| V_{H}CDR1 | GFTFSDYYMT | 48 |
| Ab/clone | | |
| AB04-411 | | |
| V_{H}CDR2 | YISGSNAYTDYADSVKG | 49 |
| Ab/clone | | |
| AB04-411 | | |
| V_{H}CDR3 | YVYQVVAVGDL | 50 |
| Ab/clone | | |
| AB04-411 | | |
| V_{L}CDR1 | RASQGISSWLA | 51 |
| Ab/clone | | |
| AB04-411 | | |
| V_{L}CDR2 | AASSLQS | 52 |
| Ab/clone | | |
| AB04-411 | | |
| V_{L}CDR3 | QQAKSFPYT | 53 |
| Ab/clone | | |
| AB04-411 | | |
| V_{H}CDR1 | GFTFSDYYMT | 54 |
| Ab/clone | | |
| AB04-482 | | |
| V_{H}CDR2 | YISGSNAYTDYADSVKG | 55 |
| Ab/clone | | |
| AB04-482 | | |
| V_{H}CDR3 | YVYQVVAIGDL | 56 |
| Ab/clone | | |
| AB04-482 | | |
| V_{L}CDR1 | RASQGISSWLA | 57 |
| Ab/clone | | |
| AB04-482 | | |
| V_{L}CDR2 | AASSLQS | 58 |
| Ab/clone | | |
| AB04-482 | | |
| V_{L}CDR3 | QQAKSFPYT | 59 |
| Ab/clone | | |
| AB04-482 | | |
| V_{H}CDR1 | GFTFSDYYMT | 60 |
| Ab/clone | | |
| AB04-276 | | |
| V_{H}CDR2 | YISGSFAYTDYADSVKG | 61 |
| Ab/clone | | |
| AB04-276 | | |
| V_{H}CDR3 | YVYQVVAIGDY | 62 |
| Ab/clone | | |
| AB04-276 | | |
| V_{L}CDR1 | RASQGISSWLA | 63 |
| Ab/clone | | |
| AB04-276 | | |
| V_{L}CDR2 | AASSLQS | 64 |
| Ab/clone | | |
| AB04-276 | | |
| V_{L}CDR3 | QQAKSFPYT | 65 |
| Ab/clone | | |
| AB04-276 | | |
| V_{H}CDR1 | GFTFSDYYMT | 66 |
| Ab/clone | | |
| AB04-483 | | |
| V_{H}CDR2 | YISGSNAYTNYADSVKG | 67 |
| Ab/clone | | |
| AB04-483 | | |
| V_{H}CDR3 | YVYQVVAIGDY | 68 |
| Ab/clone | | |
| AB04-483 | | |
| V_{L}CDR1 | RASQGISSWLA | 69 |
| Ab/clone | | |
| AB04-483 | | |
| V_{L}CDR2 | AASSLQS | 70 |
| Ab/clone | | |
| AB04-483 | | |
| V_{L}CDR3 | QQAKSFPYT | 71 |
| Ab/clone | | |
| AB04-483 | | |
| V_{H}CDR1 | GFTFSDYYMT | 72 |
| Ab/clone | | |
| AB04-365 | | |
| V_{H}CDR2 | YISGSFAYTNYADSVKG | 73 |
| Ab/clone | | |
| AB04-365 | | |
| V_{H}CDR3 | YVYQVVAIGDY | 74 |
| Ab/clone | | |
| AB04-365 | | |
| V_{L}CDR1 | RASQGISSWLA | 75 |
| Ab/clone | | |
| AB04-365 | | |
| V_{L}CDR2 | AASSLQS | 76 |
| Ab/clone | | |
| AB04-365 | | |
| V_{L}CDR3 | QQAKSFPYT | 77 |
| Ab/clone | | |
| AB04-365 | | |
| V_{H}CDR1 | GFTFSDYYMT | 78 |
| Ab/clone | | |
| AB04-151 | | |
| V_{H}CDR2 | YISGSFRYTNYADSVKG | 79 |
| Ab/clone | | |
| AB04-151 | | |
| V_{H}CDR3 | YVYQVVAIGDY | 80 |
| Ab/clone | | |
| AB04-151 | | |
| V_{L}CDR1 | RASQGISSWLA | 81 |
| Ab/clone | | |
| AB04-151 | | |
| V_{L}CDR2 | AASSLQS | 82 |
| Ab/clone | | |
| AB04-151 | | |
| V_{L}CDR3 | QQAKSFPYT | 83 |
| Ab/clone | | |
| AB04-151 | | |
| V_{H}CDR1 | GFTFSDYYMT | 84 |
| Ab/clone | | |
| AB04-160 | | |
| V_{H}CDR2 | YISGSFRYTNYADSVKG | 85 |
| Ab/clone | | |
| AB04-160 | | |
| V_{H}CDR3 | YVYQVVAIGDL | 86 |
| Ab/clone | | |
| AB04-160 | | |
| V_{L}CDR1 | RASQGISSWLA | 87 |
| Ab/clone | | |
| AB04-160 | | |
| V_{L}CDR2 | AASSLQS | 88 |
| Ab/clone | | |
| AB04-160 | | |
| V_{L}CDR3 | QQAKSFPYT | 89 |
| Ab/clone | | |
| AB04-160 | | |
| V_{H}CDR1 | GFTFSDYYMT | 90 |
| Ab/clone | | |
| AB04-439 | | |
| V_{H}CDR2 | YISGSFRYTNYADSVKG | 91 |
| Ab/clone | | |
| AB04-439 | | |
| V_{H}CDR3 | YVYQVVAIGDL | 92 |
| Ab/clone | | |
| AB04-439 | | |
| V_{L}CDR1 | RASQGISSWLA | 93 |
| Ab/clone | | |
| AB04-439 | | |
| V_{L}CDR2 | AASSLQS | 94 |
| Ab/clone | | |
| AB04-439 | | |
| V_{L}CDR3 | QQAESFPYT | 95 |
| Ab/clone | | |
| AB04-439 | | |
| V_{H}CDR1 | GFTFSDYYMT | 96 |
| Ab/clone | | |
| AB04-380 | | |
| V_{H}CDR2 | YISGSFRYTNYAPSVKG | 97 |
| Ab/clone | | |
| AB04-380 | | |
| V_{H}CDR3 | YVYQVVAIGDL | 98 |
| Ab/clone | | |
| AB04-380 | | |
| V_{L}CDR1 | RASQGISSWLA | 99 |
| Ab/clone | | |
| AB04-380 | | |
| V_{L}CDR2 | AASSLQS | 100 |
| Ab/clone | | |
| AB04-380 | | |
| V_{L}CDR3 | QQAESFPYT | 101 |
| Ab/clone | | |
| AB04-380 | | |
| V_{H}CDR1 | GFTFSDYYMT | 102 |
| Ab/clone | | |
| AB04-425 | | |
| V_{H}CDR2 | YISGSFRYTNYADKVKG | 103 |
| Ab/clone | | |
| AB04-425 | | |
| V_{H}CDR3 | YVYQVVAIGDL | 104 |
| Ab/clone | | |
| AB04-425 | | |
| V_{L}CDR1 | RASQGISSWLA | 105 |
| Ab/clone | | |
| AB04-425 | | |
| V_{L}CDR2 | AASSLQS | 106 |
| Ab/clone | | |
| AB04-425 | | |
| V_{L}CDR3 | QQAESFPYT | 107 |
| Ab/clone | | |
| AB04-425 | | |
| V_{H}CDR1 | GFTFSDYYMT | 108 |
| Ab/clone | | |
| AB04-268 | | |
| V_{H}CDR2 | YISGSFRYTNYADKVKG | 109 |
| Ab/clone | | |
| AB04-268 | | |
| V_{H}CDR3 | YVYQVVAIGDL | 110 |
| Ab/clone | | |
| AB04-268 | | |
| V_{L}CDR1 | RASQGISSWLA | 111 |
| Ab/clone | | |
| AB04-268 | | |
| V_{L}CDR2 | AASSLQS | 112 |
| Ab/clone | | |
| AB04-268 | | |
| V_{L}CDR3 | QQKESFPYT | 113 |
| Ab/clone | | |
| AB04-268 | | |
| V_{H}CDR1 | GFTFSDYYMS | 114 |
| Ab/clone | | |
| AB13-433 | | |
| V_{H}CDR2 | YISDSSTYTNYTDSVRG | 115 |
| Ab/clone | | |
| AB13-433 | | |
| V_{H}CDR3 | YLYQVIAVADS | 116 |
| Ab/clone | | |
| AB13-433 | | |
| V_{L}CDR1 | RASQGISSWLA | 117 |
| Ab/clone | | |
| AB13-433 | | |
| V_{L}CDR2 | VASNLES | 118 |
| Ab/clone | | |
| AB13-433 | | |
| V_{L}CDR3 | QQANSFPYT | 119 |
| Ab/clone | | |
| AB13-433 | | |
| V_{H}CDR1 | GFTFSDYYMS | 120 |
| Ab/clone | | |
| AB13-181 | | |
| V_{H}CDR2 | YISDSSRYTKYTDSVRG | 121 |
| Ab/clone | | |
| AB13-181 | | |
| V_{H}CDR3 | YLYQVIAIAKS | 122 |
| Ab/clone | | |
| AB13-181 | | |
| V_{L}CDR1 | RASQGISSWLA | 123 |
| Ab/clone | | |
| AB13-181 | | |
| V_{L}CDR2 | VASNLES | 124 |
| Ab/clone | | |
| AB13-181 | | |
| V_{L}CDR3 | QQANSFPYT | 125 |
| Ab/clone | | |
| AB13-181 | | |
| V_{H}CDR1 | GFTFSDYYMS | 126 |
| Ab/clone | | |
| AB13-270 | | |
| V_{H}CDR2 | YISDSSRYTNYADSVRG | 127 |
| Ab/clone | | |
| AB13-270 | | |
| V_{H}CDR3 | YLYQVIAIAKS | 128 |
| Ab/clone | | |
| AB13-270 | | |
| V_{L}CDR1 | RASQGISSWLA | 129 |
| Ab/clone | | |
| AB13-270 | | |
| V_{L}CDR2 | VASNLES | 130 |
| Ab/clone | | |
| AB13-270 | | |
| V_{L}CDR3 | QQANSFPYT | 131 |
| Ab/clone | | |
| AB13-270 | | |
| V_{H}CDR1 | GFTFSDYYMS | 132 |
| Ab/clone | | |
| AB13-147 | | |
| V_{H}CDR2 | YISDSSRYTKYTDSVRG | 133 |
| Ab/clone | | |
| AB13-147 | | |
| V_{H}CDR3 | YLYQVIAIAKS | 134 |
| Ab/clone | | |
| AB13-147 | | |
| V_{L}CDR1 | RASQGISSWLA | 135 |
| Ab/clone | | |
| AB13-147 | | |
| V_{L}CDR2 | VASNLES | 136 |
| Ab/clone | | |
| AB13-147 | | |
| V_{L}CDR3 | QQANSFPYT | 137 |
| Ab/clone | | |
| AB13-147 | | |
| V_{H}CDR1 | GFTFSDYYMS | 138 |
| Ab/clone | | |
| AB13-227 | | |
| V_{H}CDR2 | YISDSSRYTKYTDSVRG | 139 |
| Ab/clone | | |
| AB13-227 | | |
| V_{H}CDR3 | YLYQVIAIAKS | 140 |
| Ab/clone | | |
| AB13-227 | | |
| V_{L}CDR1 | RASQGISSWLA | 141 |
| Ab/clone | | |
| AB13-227 | | |
| V_{L}CDR2 | VASNLES | 142 |
| Ab/clone | | |
| AB13-227 | | |
| V_{L}CDR3 | QQAESFPYT | 143 |
| Ab/clone | | |
| AB13-227 | | |
| V_{H}CDR1 | GFTFSDYYMS | 144 |
| Ab/clone | | |
| AB13-166 | | |
| V_{H}CDR2 | YISDKSRYTKYTPSVRG | 145 |
| Ab/clone | | |
| AB13-166 | | |
| V_{H}CDR3 | YLYQVIAIADA | 146 |
| Ab/clone | | |
| AB13-166 | | |
| V_{L}CDR1 | RASQGISSWLA | 147 |
| Ab/clone | | |
| AB13-166 | | |
| V_{L}CDR2 | VASNLES | 148 |
| Ab/clone | | |
| AB13-166 | | |
| V_{L}CDR3 | QQAESFPYT | 149 |
| Ab/clone | | |
| AB13-166 | | |
| V_{H}CDR1 | GFTFSDYYMS | 150 |
| Ab/clone | | |
| AB13-288 | | |
| V_{H}CDR2 | YISDKSRYTKYTDKVRG | 151 |
| Ab/clone | | |
| AB13-288 | | |
| V_{H}CDR3 | YLYQVIAIADA | 152 |
| Ab/clone | | |
| AB13-288 | | |
| V_{L}CDR1 | RASQGISSWLA | 153 |
| Ab/clone | | |
| AB13-288 | | |
| V_{L}CDR2 | VASNLES | 154 |
| Ab/clone | | |
| AB13-288 | | |
| V_{L}CDR3 | QQAESFPYT | 155 |
| Ab/clone | | |
| AB13-288 | | |
| V_{H}CDR1 | GFTFSDYYMS | 156 |
| Ab/clone | | |
| AB13-259 | | |
| V_{H}CDR2 | YISDKSRYTKYTDSVRG | 157 |
| Ab/clone | | |
| AB13-259 | | |
| V_{H}CDR3 | YLYQVIAIADA | 158 |
| Ab/clone | | |
| AB13-259 | | |
| V_{L}CDR1 | RASQGISSWLA | 159 |
| Ab/clone | | |
| AB13-259 | | |
| V_{L}CDR2 | VASNLES | 160 |
| Ab/clone | | |
| AB13-259 | | |
| V_{L}CDR3 | QQAESFPYT | 161 |
| Ab/clone | | |
| AB13-259 | | |
| V_{H}CDR1 | GFTFSDYYMS | 162 |
| Ab/clone | | |
| AB13-459 | | |
| V_{H}CDR2 | YISDSSRYTKYTDKVRG | 163 |
| Ab/clone | | |
| AB13-459 | | |
| V_{H}CDR3 | YLYQVIAIADA | 164 |
| Ab/clone | | |
| AB13-459 | | |
| V_{L}CDR1 | RASQGISSWLA | 165 |
| Ab/clone | | |
| AB13-459 | | |
| V_{L}CDR2 | VASNLES | 166 |
| Ab/clone | | |
| AB13-459 | | |
| V_{L}CDR3 | QQAESFPYT | 167 |
| Ab/clone | | |
| AB13-459 | | |
| V_{H}CDR1 | GYTFTDYAIG | 168 |
| Ab/clone | | |
| KL31-254 | | |
| V_{H}CDR2 | DICPGDAYTNDNEKFKD | 169 |
| Ab/clone | | |
| KL31-254 | | |
| V_{H}CDR3 | GEEQVGLRNAMDY | 170 |
| Ab/clone | | |
| KL31-254 | | |
| V_{L}CDR1 | QSQSVSTSTYNYMH | 171 |
| Ab/clone | | |
| KL31-254 | | |
| V_{L}CDR2 | YASNLES | 172 |
| Ab/clone | | |
| KL31-254 | | |
| V_{L}CDR3 | QHSWEIPWT | 173 |
| Ab/clone | | |
| KL31-254 | | |
| V_{H}CDR1 | GYTFTDYCIG | 174 |
| Ab/clone | | |
| KL31-600 | | |
| V_{H}CDR2 | DICPGDAYTNDNEKFKD | 175 |
| Ab/clone | | |
| KL31-600 | | |
| V_{H}CDR3 | GEEQVGLRNAMDY | 176 |
| Ab/clone | | |
| KL31-600 | | |
| V_{L}CDR1 | QSQSVSTSTYNYMH | 177 |
| Ab/clone | | |
| KL31-600 | | |
| V_{L}CDR2 | YASNLES | 178 |
| Ab/clone | | |
| KL31-600 | | |
| V_{L}CDR3 | QHSWEIPWT | 179 |
| Ab/clone | | |
| KL31-600 | | |
| V_{H}CDR1 | GYTFTDYSIG | 180 |
| Ab/clone | | |
| KL31-515 | | |
| V_{H}CDR2 | DICPGDAYTNDNEKFKD | 181 |
| Ab/clone | | |
| KL31-515 | | |
| V_{H}CDR3 | GEEQVGLRNAMDY | 182 |
| Ab/clone | | |
| KL31-515 | | |
| V_{L}CDR1 | QSQSVSTSTYNYMH | 183 |
| Ab/clone | | |
| KL31-515 | | |
| V_{L}CDR2 | YASNLES | 184 |
| Ab/clone | | |
| KL31-515 | | |
| V_{L}CDR3 | QHSWEIPWT | 185 |
| Ab/clone | | |
| KL31-515 | | |
| V_{H}CDR1 | GYTFTDYCIG | 186 |
| Ab/clone | | |
| KL31-135 | | |
| V_{H}CDR2 | DIAPGDAYTNDNEKFKD | 187 |
| Ab/clone | | |
| KL31-135 | | |
| V_{H}CDR3 | GEEQVGLRNAMDY | 188 |
| Ab/clone | | |
| KL31-135 | | |
| V_{L}CDR1 | QSQSVSTSTYNYMH | 189 |
| Ab/clone | | |
| KL31-135 | | |
| V_{L}CDR2 | YASNLES | 190 |
| Ab/clone | | |
| KL31-135 | | |
| V_{L}CDR3 | QHSWEIPWT | 191 |
| Ab/clone | | |
| KL31-135 | | |
| V_{H}CDR1 | GYTFTDYCIG | 192 |
| Ab/clone | | |
| KL31-470 | | |
| V_{H}CDR2 | DISPGDAYTNDNEKFKD | 193 |
| Ab/clone | | |
| KL31-470 | | |
| V_{H}CDR3 | GEEQVGLRNAMDY | 194 |
| Ab/clone | | |
| KL31-470 | | |
| V_{L}CDR1 | QSQSVSTSTYNYMH | 195 |
| Ab/clone | | |
| KL31-470 | | |
| V_{L}CDR2 | YASNLES | 196 |
| Ab/clone | | |
| KL31-470 | | |
| V_{L}CDR3 | QHSWEIPWT | 197 |
| Ab/clone | | |
| KL31-470 | | |
| V_{H}CDR1 | GYTFTDYCIG | 198 |
| Ab/clone | | |
| KL31-316 | | |
| V_{H}CDR2 | DICPGDAYTNDNEKFKD | 199 |
| Ab/clone | | |
| KL31-316 | | |
| V_{H}CDR3 | GEEQVGLRNAMDY | 200 |
| Ab/clone | | |
| KL31-316 | | |
| V_{L}CDR1 | QSQSVSTSTYNYMH | 201 |
| Ab/clone | | |
| KL31-316 | | |
| V_{L}CDR2 | YASNLES | 202 |
| Ab/clone | | |
| KL31-316 | | |
| V_{L}CDR3 | QHSWEIPWT | 203 |
| Ab/clone | | |
| KL31-316 | | |
| V_{H}CDR1 | GYTFTDYCIG | 204 |
| Ab/clone | | |
| KL31-523 | | |
| V_{H}CDR2 | DICPGDAYTNDNEKFKD | 205 |
| Ab/clone | | |
| KL31-523 | | |
| V_{H}CDR3 | GEEQVGLRNAMDY | 206 |
| Ab/clone | | |
| KL31-523 | | |
| V_{L}CDR1 | QSQSVSTSTYNYMH | 207 |
| Ab/clone | | |
| KL31-523 | | |
| V_{L}CDR2 | YASNLES | 208 |
| Ab/clone | | |
| KL31-523 | | |
| V_{L}CDR3 | QHSWEIPWT | 209 |
| Ab/clone | | |
| KL31-523 | | |
| V_{H}CDR1 | GYTFTDYCIG | 210 |
| Ab/clone | | |
| KL31-567 | | |
| V_{H}CDR2 | DICPGDAYTNDNEKFKD | 211 |
| Ab/clone | | |
| KL31-567 | | |
| V_{H}CDR3 | GEEQVGLRNAMDY | 212 |
| Ab/clone | | |
| KL31-567 | | |
| V_{L}CDR1 | QSQSVSTSTYNYMH | 213 |
| Ab/clone | | |
| KL31-567 | | |
| V_{L}CDR2 | YASNLES | 214 |
| Ab/clone | | |
| KL31-567 | | |
| V_{L}CDR3 | QHSWEIPWT | 215 |
| Ab/clone | | |
| KL31-567 | | |
| V_{H}CDR1 | GYTFTDYCIG | 216 |
| Ab/clone | | |
| KL31-481 | | |
| V_{H}CDR2 | DICPGDAYTNDNEKFKD | 217 |
| Ab/clone | | |
| KL31-481 | | |
| V_{H}CDR3 | GEEQVGLRNAMDY | 218 |
| Ab/clone | | |
| KL31-481 | | |
| V_{L}CDR1 | QSQSVSTSTYNYMH | 219 |
| Ab/clone | | |
| KL31-481 | | |
| V_{L}CDR2 | YASNLES | 220 |
| Ab/clone | | |
| KL31-481 | | |
| V_{L}CDR3 | QHSWEIPWT | 221 |
| Ab/clone | | |
| KL31-481 | | |
| V_{H}CDR1 | GYTFTDYCIG | 222 |
| Ab/clone | | |
| KL31-241 | | |
| V_{H}CDR2 | DICPGDAYTNDNEKFKD | 223 |
| Ab/clone | | |
| KL31-241 | | |
| V_{H}CDR3 | GEEQLGLRNAMDY | 224 |
| Ab/clone | | |
| KL31-241 | | |
| V_{L}CDR1 | QSQSVSTSTYNYMH | 225 |
| Ab/clone | | |
| KL31-241 | | |
| V_{L}CDR2 | YASNLES | 226 |
| Ab/clone | | |
| KL31-241 | | |
| V_{L}CDR3 | QHSWEIPWT | 227 |
| Ab/clone | | |
| KL31-241 | | |
| V_{H}CDR1 | GYTFTDYCIG | 228 |
| Ab/clone | | |
| KL31-275 | | |
| V_{H}CDR2 | DICPGDAYTNDNEKFKD | 229 |
| Ab/clone | | |
| KL31-275 | | |
| V_{H}CDR3 | GEEQVGLRNAMDY | 230 |
| Ab/clone | | |
| KL31-275 | | |
| V_{L}CDR1 | QSQSVSTSTYNYMH | 231 |
| Ab/clone | | |
| KL31-275 | | |
| V_{L}CDR2 | YASNLES | 232 |
| Ab/clone | | |
| KL31-275 | | |
| V_{L}CDR3 | QHSWEIPWT | 233 |
| Ab/clone | | |
| KL31-275 | | |
| V_{H}CDR1 | GYTFTDYCIG | 234 |
| Ab/clone | | |
| KL31-313 | | |
| V_{H}CDR2 | DICPGDAYTNDNEKFKD | 235 |
| Ab/clone | | |
| KL31-313 | | |
| V_{H}CDR3 | GEEQVGLRNAMDY | 236 |
| Ab/clone | | |
| KL31-313 | | |
| V_{L}CDR1 | QSQSVSTSTYNYMH | 237 |
| Ab/clone | | |
| KL31-313 | | |
| V_{L}CDR2 | YASNLES | 238 |
| Ab/clone | | |
| KL31-313 | | |
| V_{L}CDR3 | QHSWEIPWT | 239 |
| Ab/clone | | |
| KL31-313 | | |
| V_{H}CDR1 | GYTFTDYCIG | 240 |
| Ab/clone | | |
| KL31-366 | | |
| V_{H}CDR2 | DICPGDAYTNDNEKFKD | 241 |
| Ab/clone | | |
| KL31-366 | | |
| V_{H}CDR3 | GEEQVGLRNAMDY | 242 |
| Ab/clone | | |
| KL31-366 | | |
| V_{L}CDR1 | QSQSVSTSTYNYMH | 243 |
| Ab/clone | | |
| KL31-366 | | |
| V_{L}CDR2 | YASNLES | 244 |
| Ab/clone | | |
| KL31-366 | | |
| V_{L}CDR3 | QHSWEIPWT | 245 |
| Ab/clone | | |
| KL31-366 | | |
| V_{H}CDR1 | GYTFTDYCIG | 246 |
| Ab/clone | | |
| KL31-467 | | |
| V_{H}CDR2 | DICPGDTYTNDNEKFKD | 247 |
| Ab/clone | | |
| KL31-467 | | |
| V_{H}CDR3 | GEEQLGLRNAMDY | 248 |
| Ab/clone | | |
| KL31-467 | | |
| V_{L}CDR1 | QSQSVSTSTYNYMH | 249 |
| Ab/clone | | |
| KL31-467 | | |
| V_{L}CDR2 | YASNLES | 250 |
| Ab/clone | | |
| KL31-467 | | |
| V_{L}CDR3 | QHSWEIPWT | 251 |
| Ab/clone | | |
| KL31-467 | | |
| V_{H}CDR1 | GYTFTDYCIG | 252 |
| Ab/clone | | |
| KL31-261 | | |
| V_{H}CDR2 | DICPGDVYTNDNEKFKD | 253 |
| Ab/clone | | |
| KL31-261 | | |
| V_{H}CDR3 | GEEQLGLRNAMDY | 254 |
| Ab/clone | | |
| KL31-261 | | |
| V_{L}CDR1 | QSQSVSTSTYNYMH | 255 |
| Ab/clone | | |
| KL31-261 | | |
| V_{L}CDR2 | YASNLES | 256 |
| Ab/clone | | |
| KL31-261 | | |
| V_{L}CDR3 | QHSWEIPWT | 257 |
| Ab/clone | | |
| KL31-261 | | |
| V_{H}CDR1 | GYTFTDYCIG | 258 |
| Ab/clone | | |
| KL31-356 | | |
| V_{H}CDR2 | DICPGDTYTNDNEKFKD | 259 |
| Ab/clone | | |
| KL31-356 | | |
| V_{H}CDR3 | GEEQLGLRNAMDY | 260 |
| Ab/clone | | |
| KL31-356 | | |
| V_{L}CDR1 | QSQSVSTSTYNYMH | 261 |
| Ab/clone | | |
| KL31-356 | | |
| V_{L}CDR2 | YASNLES | 262 |
| Ab/clone | | |
| KL31-356 | | |
| V_{L}CDR3 | QHSWEIPWT | 263 |
| Ab/clone | | |
| KL31-356 | | |
| V_{H}CDR1 | GYTFTDYCIG | 264 |
| Ab/clone | | |
| KL31-449 | | |
| V_{H}CDR2 | DICPGDVYTNDNEKFKD | 265 |
| Ab/clone | | |
| KL31-449 | | |
| V_{H}CDR3 | GEEQLGLRNAMDY | 266 |
| Ab/clone | | |
| KL31-449 | | |
| V_{L}CDR1 | QSQSVSTSTYNYMH | 267 |
| Ab/clone | | |
| KL31-449 | | |
| V_{L}CDR2 | YASNLES | 268 |
| Ab/clone | | |
| KL31-449 | | |
| V_{L}CDR3 | QHSWEIPWT | 269 |
| Ab/clone | | |
| KL31-449 | | |
| V_{H}CDR1 | GYTFTDYCIG | 270 |
| Ab/clone | | |
| KL31-532 | | |
| V_{H}CDR2 | DICPGDTYTNDNEKFKD | 271 |
| Ab/clone | | |
| KL31-532 | | |
| V_{H}CDR3 | GEEQLGLRNAMDY | 272 |
| Ab/clone | | |
| KL31-532 | | |
| V_{L}CDR1 | QSQSVSTSTYNYMH | 273 |
| Ab/clone | | |
| KL31-532 | | |
| V_{L}CDR2 | YASNLES | 274 |
| Ab/clone | | |
| KL31-532 | | |
| V_{L}CDR3 | GHSYEIPWT | 275 |
| Ab/clone | | |
| KL31-532 | | |
| V_{H}CDR1 | GYTFTDYCIG | 276 |
| Ab/clone | | |
| KL31-131 | | |
| V_{H}CDR2 | DICPGDTYTNDNEKFKD | 277 |
| Ab/clone | | |
| KL31-131 | | |
| V_{H}CDR3 | GEEQLGLRNAMDY | 278 |
| Ab/clone | | |
| KL31-131 | | |
| V_{L}CDR1 | QSQSVSTSTYNYMH | 279 |
| Ab/clone | | |
| KL31-131 | | |
| V_{L}CDR2 | YASNLES | 280 |
| Ab/clone | | |
| KL31-131 | | |
| V_{L}CDR3 | QHSWEIPWT | 281 |
| Ab/clone | | |
| KL31-131 | | |
| V_{H}CDR1 | GYTFTDYCIG | 282 |
| Ab/clone | | |
| KL31-478 | | |
| V_{H}CDR2 | DICPGDTYTNDNEKFKD | 283 |
| Ab/clone | | |
| KL31-478 | | |
| V_{H}CDR3 | GEEQLGLRNAMDY | 284 |
| Ab/clone | | |
| KL31-478 | | |
| V_{L}CDR1 | QSQSVSTSTYNYMH | 285 |
| Ab/clone | | |
| KL31-478 | | |
| V_{L}CDR2 | YASNLES | 286 |
| Ab/clone | | |
| KL31-478 | | |
| V_{L}CDR3 | GHSYEIPWT | 287 |
| Ab/clone | | |
| KL31-478 | | |
| V_{H}CDR1 | GYTFTDYCIG | 288 |
| Ab/clone | | |
| KL31-513 | | |
| V_{H}CDR2 | DICPGDTYTNDNEKFKD | 289 |
| Ab/clone | | |
| KL31-513 | | |
| V_{H}CDR3 | GEEQLGLRNAMDY | 290 |
| Ab/clone | | |
| KL31-513 | | |
| V_{L}CDR1 | QSQSVSTSTYNYMH | 291 |
| Ab/clone | | |
| KL31-513 | | |
| V_{L}CDR2 | YASNLES | 292 |
| Ab/clone | | |
| KL31-513 | | |
| V_{L}CDR3 | GHSYEIPWT | 293 |
| Ab/clone | | |
| KL31-513 | | |
| V_{H}CDR1 | GYTFTDYCIG | 294 |
| Ab/clone | | |
| KL31-240 | | |
| V_{H}CDR2 | DICPGDTYTNDNEKFKD | 295 |
| Ab/clone | | |
| KL31-240 | | |
| V_{H}CDR3 | GEEQLGLRNAMDY | 296 |
| Ab/clone | | |
| KL31-240 | | |
| V_{L}CDR1 | QSQSVSTSTYNYMH | 297 |
| Ab/clone | | |
| KL31-240 | | |
| V_{L}CDR2 | YASNLES | 298 |
| Ab/clone | | |
| KL31-240 | | |
| V_{L}CDR3 | GHSYEIPWT | 299 |
| Ab/clone | | |
| KL31-240 | | |
| V_{H}CDR1 | GYTFTDYCIG | 300 |
| Ab/clone | | |
| KL31-468 | | |
| V_{H}CDR2 | DICPGDTYTNDNEKFKD | 301 |
| Ab/clone | | |
| KL31-468 | | |
| V_{H}CDR3 | GEEQLGLRNAMDY | 302 |
| Ab/clone | | |
| KL31-468 | | |
| V_{L}CDR1 | QSQSVSTSTYNYMH | 303 |
| Ab/clone | | |
| KL31-468 | | |
| V_{L}CDR2 | YASNLES | 304 |
| Ab/clone | | |
| KL31-468 | | |
| V_{L}CDR3 | GHSYEIPWT | 305 |
| Ab/clone | | |
| KL31-468 | | |

TABLE A2
Exemplary Polypeptide Sequences

| | | SEQ ID |
|---|---|---|
| Description | Sequence | NO: |
| Heavy chain | QVQLVQSGAEVKKPGASVKVSCKASGYTFTDYCIGWIRQAPGQGLEWMGDI | 30 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-418 | | |
| Light chain | EIVLTQSPATLSLSPGERATLSCQSQSVSTSTYNYMHWYQQKPGQAPRLLI | 31 |
| variable | KYASNLESGIPDRFSGSGSGTDFTLTISRLEPEDFATYYCGHSYEIPWTFG | |
| region (V_{L}) | GGTKVEIK | |
| Ab/clone | | |
| KL31-418 | | |
| Heavy chain | QVQLVESGGGLVKPGGSLRLSCAASGFTFSDYYMTWIRQAPGKGLEWVSYI | 32 |
| variable | SGSFRYTNYADKVKGRFTISRDNAKNSLYLQMNSLRDEDTAVYFCARYVYQ | |
| region (V_{H}) | VVAIGDLWGQGTLVTVSS | |
| Ab/clone | | |
| AB04-168 | | |
| Light chain | DIQMTQSPSSVSASVGDRVTITCRASQGISSWLAWYQQKPGRAPKLLIFAA | 33 |
| variable | SSLQSGVPSRFSGSGSGTHFTLTISSLQPEDFATYYCQQAESFPYTFGQGT | |
| region (V_{L}) | KLEIK | |
| Ab/clone | | |
| AB04-168 | | |
| Heavy chain | QVQLVESGGGLVKPGGSLRLSCAASGFTFSDYYMSWIRQAPGKGLEWVSYI | 34 |
| variable | SDKSRYTKYTDKVRGRFTISRDNAKNSLYLQMNSLRAEDTAVYYCARYLYQ | |
| region (V_{H}) | VIAIADAWGQGTLVTVSS | |
| Ab/clone | | |
| AB13-112 | | |
| Light chain | DIQMTQSPSSVSASVGDRVTITCRASQGISSWLAWFQQKPGKAPKLLIFVA | 35 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-112 | | |
| Heavy chain | QVQLVESGGGLVKPGGSLRLSCAASGFTFSDYYMTWIRQAPGKGLEWVSYI | 306 |
| variable | SGSNAYTDYADSVKGRFTISRDNAKNSLYLQMNSLRDEDTAVYFCARYVYQ | |
| region (V_{H}) | VVAIGDYWGQGTLVTVSS | |
| Ab/clone | | |
| AB04-121 | | |
| Light chain | DIQMTQSPSSVSASVGDRVTITCRASQGISSWLAWYQQKPGRAPKLLIFAA | 307 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-121 | | |
| Heavy chain | QVQLVESGGGLVKPGGSLRLSCAASGFTFSDYYMTWIRQAPGKGLEWVSYI | 308 |
| variable | SGSNAYTDYADSVKGRFTISRDNAKNSLYLQMNSLRDEDTAVYFCARYVYQ | |
| region (V_{H}) | TVAIGDLWGQGTLVTVSS | |
| Ab/clone | | |
| AB04-174 | | |
| Light chain | DIQMTQSPSSVSASVGDRVTITCRASQGISSWLAWYQQKPGRAPKLLIFAA | 309 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-174 | | |
| Heavy chain | QVQLVESGGGLVKPGGSLRLSCAASGFTFSDYYMTWIRQAPGKGLEWVSYI | 310 |
| variable | SGSNAYTDYADSVKGRFTISRDNAKNSLYLQMNSLRDEDTAVYFCARYVYQ | |
| region (V_{H}) | VVAVGDLWGQGTLVTVSS | |
| Ab/clone | | |
| AB04-411 | | |
| Light chain | DIQMTQSPSSVSASVGDRVTITCRASQGISSWLAWYQQKPGRAPKLLIFAA | 311 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-411 | | |
| Heavy chain | QVQLVESGGGLVKPGGSLRLSCAASGFTFSDYYMTWIRQAPGKGLEWVSYI | 312 |
| variable | SGSNAYTDYADSVKGRFTISRDNAKNSLYLQMNSLRDEDTAVYFCARYVYQ | |
| region (V_{H}) | VVAIGDLWGQGTLVTVSS | |
| Ab/clone | | |
| AB04-482 | | |
| Light chain | DIQMTQSPSSVSASVGDRVTITCRASQGISSWLAWYQQKPGRAPKLLIFAA | 313 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-482 | | |
| Heavy chain | QVQLVESGGGLVKPGGSLRLSCAASGFTFSDYYMTWIRQAPGKGLEWVSYI | 314 |
| variable | SGSFAYTDYADSVKGRFTISRDNAKNSLYLQMNSLRDEDTAVYFCARYVYQ | |
| region (V_{H}) | VVAIGDYWGQGTLVTVSS | |
| Ab/clone | | |
| AB04-276 | | |
| Light chain | DIQMTQSPSSVSASVGDRVTITCRASQGISSWLAWYQQKPGRAPKLLIFAA | 315 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-276 | | |
| Heavy chain | QVQLVESGGGLVKPGGSLRLSCAASGFTFSDYYMTWIRQAPGKGLEWVSYI | 316 |
| variable | SGSNAYTNYADSVKGRFTISRDNAKNSLYLQMNSLRDEDTAVYFCARYVYQ | |
| region (V_{H}) | VVAIGDYWGQGTLVTVSS | |
| Ab/clone | | |
| AB04-483 | | |
| Light chain | DIQMTQSPSSVSASVGDRVTITCRASQGISSWLAWYQQKPGRAPKLLIFAA | 317 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-483 | | |
| Heavy chain | QVQLVESGGGLVKPGGSLRLSCAASGFTFSDYYMTWIRQAPGKGLEWVSYI | 318 |
| variable | SGSFAYTNYADSVKGRFTISRDNAKNSLYLQMNSLRDEDTAVYFCARYVYQ | |
| region (V_{H}) | VVAIGDYWGQGTLVTVSS | |
| Ab/clone | | |
| AB04-365 | | |
| Light chain | DIQMTQSPSSVSASVGDRVTITCRASQGISSWLAWYQQKPGRAPKLLIFAA | 319 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-365 | | |
| Heavy chain | QVQLVESGGGLVKPGGSLRLSCAASGFTFSDYYMTWIRQAPGKGLEWVSYI | 320 |
| variable | SGSFRYTNYADSVKGRFTISRDNAKNSLYLQMNSLRDEDTAVYFCARYVYQ | |
| region (V_{H}) | VVAIGDYWGQGTLVTVSS | |
| Ab/clone | | |
| AB04-151 | | |
| Light chain | DIQMTQSPSSVSASVGDRVTITCRASQGISSWLAWYQQKPGRAPKLLIFAA | 321 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-151 | | |
| Heavy chain | QVQLVESGGGLVKPGGSLRLSCAASGFTFSDYYMTWIRQAPGKGLEWVSYI | 322 |
| variable | SGSFRYTNYADSVKGRFTISRDNAKNSLYLQMNSLRDEDTAVYFCARYVYQ | |
| region (V_{H}) | VVAIGDLWGQGTLVTVSS | |
| Ab/clone | | |
| AB04-160 | | |
| Light chain | DIQMTQSPSSVSASVGDRVTITCRASQGISSWLAWYQQKPGRAPKLLIFAA | 323 |
| variable | SSLQSGVPSRFSGSGSGTHFTLTISSLQPEDFATYYCQQAKSFPYTFGQGT | |
| region (V_{L}) | KLEIK | |
| Ab/clone | | |
| AB04-160 | | |
| Heavy chain | QVQLVESGGGLVKPGGSLRLSCAASGFTFSDYYMTWIRQAPGKGLEWVSYI | 324 |
| variable | SGSFRYTNYADSVKGRFTISRDNAKNSLYLQMNSLRDEDTAVYFCARYVYQ | |
| region (V_{H}) | VVAIGDLWGQGTLVTVSS | |
| Ab/clone | | |
| AB04-439 | | |
| Light chain | DIQMTQSPSSVSASVGDRVTITCRASQGISSWLAWYQQKPGRAPKLLIFAA | 325 |
| variable | SSLQSGVPSRFSGSGSGTHFTLTISSLQPEDFATYYCQQAESFPYTFGQGT | |
| region (V_{L}) | KLEIK | |
| Ab/clone | | |
| AB04-439 | | |
| Heavy chain | QVQLVESGGGLVKPGGSLRLSCAASGFTFSDYYMTWIRQAPGKGLEWVSYI | 326 |
| variable | SGSFRYTNYAPSVKGRFTISRDNAKNSLYLQMNSLRDEDTAVYFCARYVYQ | |
| region (V_{H}) | VVAIGDLWGQGTLVTVSS | |
| Ab/clone | | |
| AB04-380 | | |
| Light chain | DIQMTQSPSSVSASVGDRVTITCRASQGISSWLAWYQQKPGRAPKLLIFAA | 327 |
| variable | SSLQSGVPSRFSGSGSGTHFTLTISSLQPEDFATYYCQQAESFPYTFGQGT | |
| region (V_{L}) | KLEIK | |
| Ab/clone | | |
| AB04-380 | | |
| Heavy chain | QVQLVESGGGLVKPGGSLRLSCAASGFTFSDYYMTWIRQAPGKGLEWVSYI | 328 |
| variable | SGSFRYTNYADKVKGRFTISRDNAKNSLYLQMNSLRDEDTAVYFCARYVYQ | |
| region (V_{H}) | VVAIGDLWGQGTLVTVSS | |
| Ab/clone | | |
| AB04-425 | | |
| Light chain | DIQMTQSPSSVSASVGDRVTITCRASQGISSWLAWYQQKPGRAPKLLIFAA | 329 |
| variable | SSLQSGVPSRFSGSGSGTHFTLTISSLQPEDFATYYCQQAESFPYTFGQGT | |
| region (V_{L}) | KLEIK | |
| Ab/clone | | |
| AB04-425 | | |
| Heavy chain | QVQLVESGGGLVKPGGSLRLSCAASGFTFSDYYMTWIRQAPGKGLEWVSYI | 330 |
| variable | SGSFRYTNYADKVKGRFTISRDNAKNSLYLQMNSLRDEDTAVYFCARYVYQ | |
| region (V_{H}) | VVAIGDLWGQGTLVTVSS | |
| Ab/clone | | |
| AB04-268 | | |
| Light chain | DIQMTQSPSSVSASVGDRVTITCRASQGISSWLAWYQQKPGRAPKLLIFAA | 331 |
| variable | SSLQSGVPSRFSGSGSGTHFTLTISSLQPEDFATYYCQQKESFPYTFGQGT | |
| region (V_{L}) | KLEIK | |
| Ab/clone | | |
| AB04-268 | | |
| Heavy chain | QVQLVESGGGLVKPGGSLRLSCAASGFTFSDYYMSWIRQAPGKGLEWVSYI | 332 |
| variable | SDSSTYTNYTDSVRGRFTISRDNAKNSLYLQMNSLRAEDTAVYYCARYLYQ | |
| region (V_{H}) | VIAVADSWGQGTLVTVSS | |
| Ab/clone | | |
| AB13-433 | | |
| Light chain | DIQMTQSPSSVSASVGDRVTITCRASQGISSWLAWFQQKPGKAPKLLIYVA | 333 |
| variable | SNLESGVPSRFSGSGSGTDFTLTISSLQPEDFATYYCQQANSFPYTFGQGT | |
| region (V_{L}) | KLEIK | |
| Ab/clone | | |
| AB13-433 | | |
| Heavy chain | QVQLVESGGGLVKPGGSLRLSCAASGFTFSDYYMSWIRQAPGKGLEWVSYI | 334 |
| variable | SDSSRYTKYTDSVRGRFTISRDNAKNSLYLQMNSLRAEDTAVYYCARYLYQ | |
| region (V_{H}) | VIAIAKSWGQGTLVTVSS | |
| Ab/clone | | |
| AB13-181 | | |
| Light chain | DIQMTQSPSSVSASVGDRVTITCRASQGISSWLAWFQQKPGKAPKLLIYVA | 335 |
| variable | SNLESGVPSRFSGSGSGTDFTLTISSLQPEDFATYYCQQANSFPYTFGQGT | |
| region (V_{L}) | KLEIK | |
| Ab/clone | | |
| AB13-181 | | |
| Heavy chain | QVQLVESGGGLVKPGGSLRLSCAASGFTFSDYYMSWIRQAPGKGLEWVSYI | 336 |
| variable | SDSSRYTNYADSVRGRFTISRDNAKNSLYLQMNSLRAEDTAVYYCARYLYQ | |
| region (V_{H}) | VIAIAKSWGQGTLVTVSS | |
| Ab/clone | | |
| AB13-270 | | |
| Light chain | DIQMTQSPSSVSASVGDRVTITCRASQGISSWLAWFQQKPGKAPKLLIYVA | 337 |
| variable | SNLESGVPSRFSGSGSGTDFTLTISSLQPEDFATYYCQQANSFPYTFGQGT | |
| region (V_{L}) | KLEIK | |
| Ab/clone | | |
| AB13-270 | | |
| Heavy chain | QVQLVESGGGLVKPGGSLRLSCAASGFTFSDYYMSWIRQAPGKGLEWVSYI | 338 |
| variable | SDSSRYTKYTDSVRGRFTISRDNAKNSLYLQMNSLRAEDTAVYYCARYLYQ | |
| region (V_{H}) | VIAIAKSWGQGTLVTVSS | |
| Ab/clone | | |
| AB13-147 | | |
| Light chain | DIQMTQSPSSVSASVGDRVTITCRASQGISSWLAWFQQKPGKAPKLLIFVA | 339 |
| variable | SNLESGVPSRFSGSGSGTDFTLTISSLQPEDFATYYCQQANSFPYTFGQGT | |
| region (V_{L}) | KLEIK | |
| Ab/clone | | |
| AB13-147 | | |
| Heavy chain | QVQLVESGGGLVKPGGSLRLSCAASGFTFSDYYMSWIRQAPGKGLEWVSYI | 340 |
| variable | SDSSRYTKYTDSVRGRFTISRDNAKNSLYLQMNSLRAEDTAVYYCARYLYQ | |
| region (V_{H}) | VIAIAKSWGQGTLVTVSS | |
| Ab/clone | | |
| AB13-227 | | |
| Light chain | DIQMTQSPSSVSASVGDRVTITCRASQGISSWLAWFQQKPGKAPKLLIFVA | 341 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-227 | | |
| Heavy chain | QVQLVESGGGLVKPGGSLRLSCAASGFTFSDYYMSWIRQAPGKGLEWVSYI | 342 |
| variable | SDKSRYTKYTPSVRGRFTISRDNAKNSLYLQMNSLRAEDTAVYYCARYLYQ | |
| region (V_{H}) | VIAIADAWGQGTLVTVSS | |
| Ab/clone | | |
| AB13-166 | | |
| Light chain | DIQMTQSPSSVSASVGDRVTITCRASQGISSWLAWFQQKPGKAPKLLIFVA | 343 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-166 | | |
| Heavy chain | QVQLVESGGGLVKPGGSLRLSCAASGFTFSDYYMSWIRQAPGKGLEWVSYI | 344 |
| variable | SDKSRYTKYTDKVRGRFTISRDNAKNSLYLQMNSLRAEDTAVYYCARYLYQ | |
| region (V_{H}) | VIAIADAWGQGTLVTVSS | |
| Ab/clone | | |
| AB13-288 | | |
| Light chain | DIQMTQSPSSVSASVGDRVTITCRASQGISSWLAWFQQKPGKAPKLLIFVA | 345 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-288 | | |
| Heavy chain | QVQLVESGGGLVKPGGSLRLSCAASGFTFSDYYMSWIRQAPGKGLEWVSYI | 346 |
| variable | SDKSRYTKYTDSVRGRFTISRDNAKNSLYLQMNSLRAEDTAVYYCARYLYQ | |
| region (V_{H}) | VIAIADAWGQGTLVTVSS | |
| Ab/clone | | |
| AB13-259 | | |
| Light chain | DIQMTQSPSSVSASVGDRVTITCRASQGISSWLAWFQQKPGKAPKLLIFVA | 347 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-259 | | |
| Heavy chain | QVQLVESGGGLVKPGGSLRLSCAASGFTFSDYYMSWIRQAPGKGLEWVSYI | 348 |
| variable | SDSSRYTKYTDKVRGRFTISRDNAKNSLYLQMNSLRAEDTAVYYCARYLYQ | |
| region (V_{H}) | VIAIADAWGQGTLVTVSS | |
| Ab/clone | | |
| AB13-459 | | |
| Light chain | DIQMTQSPSSVSASVGDRVTITCRASQGISSWLAWFQQKPGKAPKLLIFVA | 349 |
| variable | SNLESGVPSRFSGSGSGTDFTLTISSLQPEDFATYYCQQAESFPYTFGQGT | |
| region (V_{L}) | KLEIK | |
| Ab/clone | | |
| AB13-459 | | |
| Heavy chain | QVQLQQSGAELVRPGSSVRMSCKAVGYTFTDYAIGWIKQRPGHGLEWIGDI | 350 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-254 | | |
| Light chain | DIVLTQSPASLAVSLGQRATISCQSQSVSTSTYNYMHWYQQKPGQPPKLLI | 351 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-254 | | |
| Heavy chain | QVQLQQSGAELVRPGSSVRMSCKAVGYTFTDYCIGWIKQRPGHGLEWIGDI | 352 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-600 | | |
| Light chain | DIVLTQSPASLAVSLGQRATISCQSQSVSTSTYNYMHWYQQKPGQPPKLLI | 353 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-600 | | |
| Heavy chain | QVQLQQSGAELVRPGSSVRMSCKAVGYTFTDYSIGWIKQRPGHGLEWIGDI | 354 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-515 | | |
| Light chain | DIVLTQSPASLAVSLGQRATISCQSQSVSTSTYNYMHWYQQKPGQPPKLLI | 355 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-515 | | |
| Heavy chain | QVQLQQSGAELVRPGSSVRMSCKAVGYTFTDYCIGWIKQRPGHGLEWIGDI | 356 |
| variable | APGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-135 | | |
| Light chain | DIVLTQSPASLAVSLGQRATISCQSQSVSTSTYNYMHWYQQKPGQPPKLLI | 357 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-135 | | |
| Heavy chain | QVQLQQSGAELVRPGSSVRMSCKAVGYTFTDYCIGWIKQRPGHGLEWIGDI | 358 |
| variable | SPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-470 | | |
| Light chain | DIVLTQSPASLAVSLGQRATISCQSQSVSTSTYNYMHWYQQKPGQPPKLLI | 359 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-470 | | |
| Heavy chain | QVQLVQSGAEVKKPGASVKVSCKASGYTFTDYCIGWVRQAPGQGLEWMGDI | 360 |
| variable | CPGDAYTNDNEKFKDRVTMTRDTSTSTVYMELSSLRSEDTAVYYCARGEEQ | |
| region (V_{H}) | VGLRNAMDYWGQGTSVTVSS | |
| Ab/clone | | |
| KL31-316 | | |
| Light chain | DIVMTQSPDSLAVSLGERATINCQSQSVSTSTYNYMHWYQQKPGQPPKLLI | 361 |
| variable | YYASNLESGVPDRFSGSGSGTDFTLTISSLQAEDVAVYYCQHSWEIPWTFG | |
| region (V_{L}) | GGTKLEIK | |
| Ab/clone | | |
| KL31-316 | | |
| Heavy chain | QVQLVQSGAEVKKPGASVKVSCKASGYTFTDYCIGWIRQAPGQGLEWMGDI | 362 |
| variable | CPGDAYTNDNEKFKDRATMTADTSTSTAYMELSSLRSEDTAVYYCARGEEQ | |
| region (V_{H}) | VGLRNAMDYWGQGTSVTVSS | |
| Ab/clone | | |
| KL31-523 | | |
| Light chain | DIVMTQSPDSLAVSLGERATINCQSQSVSTSTYNYMHWYQQKPGQPPKLLI | 363 |
| variable | KYASNLESGVPDRFSGSGSGTDFTLTISSLQAEDVATYYCQHSWEIPWTFG | |
| region (V_{L}) | GGTKLEIK | |
| Ab/clone | | |
| KL31-523 | | |
| Heavy chain | QVQLVQSGAEVKKPGASVKVSCKASGYTFTDYCIGWIRQAPGQGLEWMGDI | 364 |
| variable | CPGDAYTNDNEKFKDRATMTADTSTSTAYMELSSLRSEDTAVYYCARGEEQ | |
| region (V_{H}) | VGLRNAMDYWGQGTSVTVSS | |
| Ab/clone | | |
| KL31-567 | | |
| Light chain | DIVMTQSPDSLAVSLGERATINCQSQSVSTSTYNYMHWYQQKPGQPPKLLI | 365 |
| variable | YYASNLESGVPDRFSGSGSGTDFTLTISSLQAEDVAVYYCQHSWEIPWTFG | |
| region (V_{L}) | GGTKLEIK | |
| Ab/clone | | |
| KL31-567 | | |
| Heavy chain | QVQLVQSGAEVKKPGASVKVSCKASGYTFTDYCIGWVRQAPGQGLEWMGDI | 366 |
| variable | CPGDAYTNDNEKFKDRVTMTRDTSTSTVYMELSSLRSEDTAVYYCARGEEQ | |
| region (V_{H}) | VGLRNAMDYWGQGTSVTVSS | |
| Ab/clone | | |
| KL31-481 | | |
| Light chain | DIVMTQSPDSLAVSLGERATINCQSQSVSTSTYNYMHWYQQKPGQPPKLLI | 367 |
| variable | KYASNLESGVPDRFSGSGSGTDFTLTISSLQAEDVATYYCQHSWEIPWTFG | |
| region (V_{L}) | GGTKLEIK | |
| Ab/clone | | |
| KL31-481 | | |
| Heavy chain | QVQLVQSGAEVKKPGASVKVSCKASGYTFTDYCIGWIRQAPGQGLEWMGDI | 368 |
| variable | CPGDAYTNDNEKFKDRATMTADTSTSTAYMELSSLRSEDTAVYYCARGEEQ | |
| region (V_{H}) | LGLRNAMDYWGQGTSVTVSS | |
| Ab/clone | | |
| KL31-241 | | |
| Light chain | DIVMTQSPDSLAVSLGERATINCQSQSVSTSTYNYMHWYQQKPGQPPKLLI | 369 |
| variable | KYASNLESGVPDRFSGSGSGTDFTLTISSLQAEDVATYYCQHSWEIPWTFG | |
| region (V_{L}) | GGTKLEIK | |
| Ab/clone | | |
| KL31-241 | | |
| Heavy chain | QVQLQQSGAELVRPGSSVRMSCKAVGYTFTDYCIGWIKQRPGHGLEWIGDI | 370 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-275 | | |
| Light chain | DIVLTQSPASLAVSLGQRATISCQSQSVSTSTYNYMHWYQQKPGQPPKLLI | 371 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-275 | | |
| Heavy chain | QVQLQQSGAELVRPGSSVRMSCKAVGYTFTDYCIGWIKQRPGHGLEWIGDI | 372 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-313 | | |
| Light chain | DIVLTQSPASLAVSLGQRATISCQSQSVSTSTYNYMHWYQQKPGQPPKLLI | 373 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-313 | | |
| Heavy chain | QVQLQQSGAELVRPGSSVRMSCKAVGYTFTDYCIGWIKQRPGHGLEWIGDI | 374 |
| variable | CPGDAYTNDNEKFKDKATLTADTSSTTAYMQLSSLTSEDSAIYYCARGEEQ | |
| region (V_{H}) | VGLRNAMDYWGQGTSVTVSS | |
| Ab/clone | | |
| KL31-366 | | |
| Light chain | DIVLTQSPASLAVSLGQRATISCQSQSVSTSTYNYMHWYQQKPGQPPKLLI | 375 |
| variable | KYASNLESGVPARFSGSGSGTDFTLNIHPVEEEDTATYYCQHSWEIPWTFG | |
| region (V_{L}) | GGTKLEIK | |
| Ab/clone | | |
| KL31-366 | | |
| Heavy chain | QVQLVQSGAEVKKPGASVKVSCKASGYTFTDYCIGWIRQAPGQGLEWMGDI | 376 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-467 | | |
| Light chain | DIVMTQSPDSLAVSLGERATINCQSQSVSTSTYNYMHWYQQKPGQPPKLLI | 377 |
| variable | KYASNLESGVPDRFSGSGSGTDFTLTISSLQAEDVATYYCQHSWEIPWTFG | |
| region (V_{L}) | GGTKVEIK | |
| Ab/clone | | |
| KL31-467 | | |
| Heavy chain | QVQLVQSGAEVKKPGASVKVSCKASGYTFTDYCIGWIRQAPGQGLEWMGDI | 378 |
| variable | CPGDVYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-261 | | |
| Light chain | DIVMTQSPDSLAVSLGERATINCQSQSVSTSTYNYMHWYQQKPGQPPKLLI | 379 |
| variable | KYASNLESGVPDRFSGSGSGTDFTLTISSLQAEDVATYYCQHSWEIPWTFG | |
| region (V_{L}) | GGTKVEIK | |
| Ab/clone | | |
| KL31-261 | | |
| Heavy chain | QVQLVQSGAEVKKPGASVKVSCKASGYTFTDYCIGWIRQAPGQGLEWMGDI | 380 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-356 | | |
| Light chain | DIVMTQSPDSLAVSLGERATINCQSQSVSTSTYNYMHWYQQKPGQPPKLLI | 381 |
| variable | KYASNLESGVPDRFSGSGSGTDFTLTISSLQAEDVATYYCQHSWEIPWTFG | |
| region (V_{L}) | GGTKVEIK | |
| Ab/clone | | |
| KL31-356 | | |
| Heavy chain | QVQLVQSGAEVKKPGASVKVSCKASGYTFTDYCIGWIRQAPGQGLEWMGDI | 382 |
| variable | CPGDVYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-449 | | |
| Light chain | DIVMTQSPDSLAVSLGERATINCQSQSVSTSTYNYMHWYQQKPGQPPKLLI | 383 |
| variable | KYASNLESGVPDRFSGSGSGTDFTLTISSLQAEDVATYYCQHSWEIPWTFG | |
| region (V_{L}) | GGTKVEIK | |
| Ab/clone | | |
| KL31-449 | | |
| Heavy chain | QVQLVQSGAEVKKPGASVKVSCKASGYTFTDYCIGWIRQAPGQGLEWMGDI | 384 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-532 | | |
| Light chain | DIVMTQSPDSLAVSLGERATINCQSQSVSTSTYNYMHWYQQKPGQPPKLLI | 385 |
| variable | KYASNLESGVPDRFSGSGSGTDFTLTISSLQAEDVATYYCGHSYEIPWTFG | |
| region (V_{L}) | GGTKVEIK | |
| Ab/clone | | |
| KL31-532 | | |
| Heavy chain | QVQLVQSGAEVKKPGASVKVSCKASGYTFTDYCIGWIRQAPGQGLEWMGDI | 386 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-131 | | |
| Light chain | EIVLTQSPATLSLSPGERATLSCQSQSVSTSTYNYMHWYQQKPGQAPRLLI | 387 |
| variable | KYASNLESGIPDRFSGSGSGTDFTLTISRLEPEDFATYYCQHSWEIPWTFG | |
| region (V_{L}) | GGTKVEIK | |
| Ab/clone | | |
| KL31-131 | | |
| Heavy chain | QVQLVQSGAEVKKPGASVKVSCKASGYTFTDYCIGWIRQAPGQGLEWMGDI | 388 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-478 | | |
| Light chain | EIVLTQSPATLSLSPGERATLSCQSQSVSTSTYNYMHWYQQKPGQAPRLLI | 389 |
| variable | KYASNLESGIPDRFSGSGSGTDFTLTISRLEPEDFATYYCGHSYEIPWTFG | |
| region (V_{L}) | GGTKVEIK | |
| Ab/clone | | |
| KL31-478 | | |
| Heavy chain | QVQLVQSGAEVKKPGASVKVSCKASGYTFTDYCIGWIRQAPGQGLEWMGDI | 390 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-513 | | |
| Light chain | EIVLTQSPATLSLSPGERATLSCQSQSVSTSTYNYMHWYQQKPGQAPRLLI | 391 |
| variable | KYASNLESGIPDRFSGSGSGTDFTLTISRLEPEDFATYYCGHSYEIPWTFG | |
| region (V_{L}) | GGTKVEIK | |
| Ab/clone | | |
| KL31-513 | | |
| Heavy chain | QVQLVQSGAEVKKPGASVKVSCKASGYTFTDYCIGWIRQAPGQGLEWMGDI | 392 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-240 | | |
| Light chain | EIVLTQSPATLSLSPGERATLSCQSQSVSTSTYNYMHWYQQKPGQAPRLLI | 393 |
| variable | KYASNLESGIPDRFSGSGSGTDFTLTISRLEPEDFATYYCGHSYEIPWTFG | |
| region (V_{L}) | GGTKVEIK | |
| Ab/clone | | |
| KL31-240 | | |
| Heavy chain | QVQLVQSGAEVKKPGASVKVSCKASGYTFTDYCIGWIRQAPGQGLEWMGDI | 394 |
| variable | CPGDTYTNDNEKFKDRATMTADTSTSTAYMELSSLRSEDTAVYYCARGEEQ | |
| region (V_{H}) | LGLRNAMDYWGQGTLVTVSS | |
| Ab/clone | | |
| KL31-468 | | |
| Light chain | EIVLTQSPATLSLSPGERATLSCQSQSVSTSTYNYMHWYQQKPGQAPRLLI | 395 |
| variable | KYASNLESGIPDRFSGSGSGTDFTLTISRLEPEDFATYYCGHSYEIPWTFG | |
| region (V_{L}) | GGTKVEIK | |
| Ab/clone | | |
| KL31-468 | | |

TABLE A3
CDR Consensus Sequence Amino Acids

| X₁₋₄₃ | Amino Acids | X₄₄₋₈₆ | Amino Acids |
|---|---|---|---|
| X₁ | S, R, T, N | X₄₄ | N, T |
| X₂ | D, A, E | X₄₅ | D, A, E, V |
| X₃ | M, V | X₄₆ | G, I, P |
| X₄ | T, F, H, K, S, L, V, Y | X₄₇ | E, Q, V |
| X₅ | I, L, T, V | X₄₈ | E, G |
| X₆ | R, G, S | X₄₉ | Q, A, I, T |
| X₇ | N, A, F, G, H, K, L, Q, R, | X₅₀ | V, D, E, I, L, P, T, Y |
| | S, T, V, Y | | |
| X₈ | A, F, H, K, Q, R, S, V, Y | X₅₁ | G, R, P, T, V |
| X₉ | T, I | X₅₂ | R, T |
| X₁₀ | N, S, D | X₅₃ | N, P |
| X₁₁ | Y, L, N | X₅₄ | A, N, D, Q, H, I, L, K |
| X₁₂ | D, P | X₅₅ | M, R |
| X₁₃ | S, K | X₅₆ | D, G, H |
| X₁₄ | V, Y, L, I, F | X₅₇ | Y, A, N, H, I, K, S, T |
| X₁₅ | T, V, I, A | X₅₈ | Q, G |
| X₁₆ | V, L, I | X₅₉ | H, Q, P |
| X₁₇ | A, S | X₆₀ | S, A, D, E, F, H, K, L, |
| | | | P, R, Y |
| X₁₈ | V, Y, T, S, Q, N, L, I, F | X₆₁ | W, D, E, F, P, Y |
| X₁₉ | G, V, T, P, N, L, I, A | X₆₂ | E, R, S, T, V |
| X₂₀ | D, E | X₆₃ | I, V, L |
| X₂₁ | Y, V, T, S, R, Q, N, L, K, | X₆₄ | P, D, F, K, N, Q, R, Y |
| | I, H, G, F, E, D, A | | |
| X₂₂ | S, Q | X₆₅ | W, D, E, F, Y |
| X₂₃ | Q, E, H, T | X₆₆ | T, N, Q, R |
| X₂₄ | A, I, K, L, S | X₆₇ | S, T |
| X₂₅ | K, A, D, E, F, G, H, I, L, | X₆₈ | D, E, Y |
| | N, Q, R, S, T, V | | |
| X₂₆ | S, A, E, F, H, K, Q, R | X₆₉ | S, A, F, H, K, L, P, Q, |
| | | | R, V, Y |
| X₂₇ | P, K, R, S, I, L, V, T | X₇₀ | S, N |
| X₂₈ | Y, N | X₇₁ | T, A, R |
| X₂₉ | T, A, D, E, F, G, H, I K, | X₇₂ | N, K |
| | L, N, Q, R, S, V, Y | | |
| X₃₀ | T, A, I, S | X₇₃ | T, A |
| X₃₁ | D, H, P, Q, Y | X₇₄ | D, A, E, G, H, I, K, L, |
| | | | N, P, Q, R, S, T, V, Y |
| X₃₂ | Y, F | X₇₅ | S, A, D, F, H, I, K, L, |
| | | | N, P, Q, R, T, V |
| X₃₃ | I, L, T, V | X₇₆ | Y, H, I, F, V |
| X₃₄ | G, F, I, K, L, T, Y | X₇₇ | L, A, I, K, S, T, V |
| X₃₅ | D, G, Y | X₇₈ | H, A, F, V, Y |
| X₃₆ | I, M, V | X₇₉ | Q, D, E |
| X₃₇ | P, L | X₈₀ | I, R, P, V |
| X₃₈ | G, I, K, R | X₈₁ | A, I, K, R |
| X₃₉ | D, E, I, V | X₈₂ | V, I, L, F |
| X₄₀ | A, E, I, P, Q, S, T, V | X₈₃ | A, F, I, K, P, Q, S, T, |
| | | | V, Y |
| X₄₁ | Y, E, F, H, K, P, Q, R, S, | X₈₄ | D, E, F, G, H, I, K, L, |
| | T, V | | N, R, S, T, V, Y |
| X₄₂ | T, A, S | X₈₅ | S, A, D, E, F, G, H, I, K, |
| | | | L, N, P, Q, R, T, V, Y |
| X₄₃ | N, K | X₈₆ | N, K, E |

Thus, in certain embodiments, an antibody or antigen-binding fragment thereof comprises

a heavy chain variable region (V_{H}) sequence that comprises complementary determining region V_{H}CDR1, V_{H}CDR2, and V_{H}CDR3 sequences selected from Table A1 and variants thereof which specifically bind to a human HRS polypeptide (selected, for example, from Table H); and

a light chain variable region (V_{L}) sequence that comprises complementary determining region V_{L}CDR1, V_{L}CDR2, and V_{L}CDR3 sequences selected from Table A1 and variants thereof which specifically bind to the human HRS polypeptide (selected, for example, from Table H).

Also included are affinity matured variants of an antibody or antigen-binding fragment thereof.

In certain embodiments, the CDR sequences are as follows:

the V_{H}CDR1, V_{H}CDR2, and V_{H}CDR3 sequences comprise the consensus sequences of SEQ ID NOs:396, 397, and 398 (as defined in Table A3), respectively, and the V_{L}CDR1, V_{L}CDR2, and V_{L}CDR3 sequences comprise the consensus sequences SEQ ID NOs: 399, 400, and 401 (as defined in Table A3), respectively, including variants thereof;

the V_{H}CDR1, V_{H}CDR2, and V_{H}CDR3 sequences comprise the consensus sequences of SEQ ID NOs: 402, 403, and 404 (as defined in Table A3), respectively, and the V_{L}CDR1, V_{L}CDR2, and V_{L}CDR3 sequences comprise the consensus sequences of SEQ ID NOs: 405, 406, and 407 (as defined in Table A3), respectively, including variants thereof;

the V_{H}CDR1, V_{H}CDR2, and V_{H}CDR3 sequences comprise the consensus sequences of SEQ ID NOs: 408, 409, and 410 (as defined in Table A3), respectively, and the V_{L}CDR1, V_{L}CDR2, and V_{L}CDR3 sequences comprise the consensus sequences of SEQ ID NOs: 411, 412, and 413 (as defined in Table A3), respectively, including variants thereof;

the V_{H}CDR1, V_{H}CDR2, and V_{H}CDR3 sequences comprise SEQ ID NOs: 12, 13, and 14, respectively, and the V_{L}CDR1, V_{L}CDR2, and V_{L}CDR3 sequences comprise SEQ ID NOs: 15, 16, and 17, respectively, including variants thereof;

the V_{H}CDR1, V_{H}CDR2, and V_{H}CDR3 sequences comprise SEQ ID NOs: 18, 19, and 20, respectively, and the V_{L}CDR1, V_{L}CDR2, and V_{L}CDR3 sequences comprise SEQ ID NOs: 21, 22, and 23, respectively, including variants thereof;

the V_{H}CDR1, V_{H}CDR2, and V_{H}CDR3 sequences comprise SEQ ID NOs: 24, 25, and 26, respectively, and the V_{L}CDR1, V_{L}CDR2, and V_{L}CDR3 sequences comprise SEQ ID NOs: 27, 28, and 29, respectively, including variants thereof;

the V_{H}CDR1, V_{H}CDR2, and V_{H}CDR3 sequences comprise SEQ ID NOs: 36, 37, and 38, respectively, and the V_{L}CDR1, V_{L}CDR2, and V_{L}CDR3 sequences comprise SEQ ID NOs: 39, 40, and 41, respectively, including variants thereof;

the V_{H}CDR1, V_{H}CDR2, and V_{H}CDR3 sequences comprise SEQ ID NOs: 42, 43, and 44, respectively, and the V_{L}CDR1, V_{L}CDR2, and V_{L}CDR3 sequences comprise SEQ ID NOs: 45, 46, and 47, respectively, including variants thereof;

the V_{H}CDR1, V_{H}CDR2, and V_{H}CDR3 sequences comprise SEQ ID NOs: 48, 49, and 50, respectively, and the V_{L}CDR1, V_{L}CDR2, and V_{L}CDR3 sequences comprise SEQ ID NOs: 51, 52, and 53, respectively, including variants thereof;

the V_{H}CDR1, V_{H}CDR2, and V_{H}CDR3 sequences comprise SEQ ID NOs: 54, 55, and 56, respectively, and the V_{L}CDR1, V_{L}CDR2, and V_{L}CDR3 sequences comprise SEQ ID NOs: 57, 58, and 59, respectively, including variants thereof;

the V_{H}CDR1, V_{H}CDR2, and V_{H}CDR3 sequences comprise SEQ ID NOs: 60, 61, and 62, respectively, and the V_{L}CDR1, V_{L}CDR2, and V_{L}CDR3 sequences comprise SEQ ID NOs: 63, 64, and 65, respectively, including variants thereof;

the V_{H}CDR1, V_{H}CDR2, and V_{H}CDR3 sequences comprise SEQ ID NOs: 66, 67, and 68, respectively, and the V_{L}CDR1, V_{L}CDR2, and V_{L}CDR3 sequences comprise SEQ ID NOs: 69, 70, and 71, respectively, including variants thereof;

the V_{H}CDR1, V_{H}CDR2, and V_{H}CDR3 sequences comprise SEQ ID NOs: 72, 73, and 74, respectively, and the V_{L}CDR1, V_{L}CDR2, and V_{L}CDR3 sequences comprise SEQ ID NOs: 75, 76, and 77, respectively, including variants thereof;

the V_{H}CDR1, V_{H}CDR2, and V_{H}CDR3 sequences comprise SEQ ID NOs: 78, 79, and 80, respectively, and the V_{L}CDR1, V_{L}CDR2, and V_{L}CDR3 sequences comprise SEQ ID NOs: 81, 82, and 83, respectively, including variants thereof;

the V_{H}CDR1, V_{H}CDR2, and V_{H}CDR3 sequences comprise SEQ ID NOs: 84, 85, and 86, respectively, and the V_{L}CDR1, V_{L}CDR2, and V_{L}CDR3 sequences comprise SEQ ID NOs: 87, 88, and 89, respectively, including variants thereof;

the V_{H}CDR1, V_{H}CDR2, and V_{H}CDR3 sequences comprise SEQ ID NOs: 90, 91, and 92, respectively, and the V_{L}CDR1, V_{L}CDR2, and V_{L}CDR3 sequences comprise SEQ ID NOs: 93, 94, and 95, respectively, including variants thereof;

the V_{H}CDR1, V_{H}CDR2, and V_{H}CDR3 sequences comprise SEQ ID NOs: 96, 97, and 98, respectively, and the V_{L}CDR1, V_{L}CDR2, and V_{L}CDR3 sequences comprise SEQ ID NOs: 99, 100, and 101, respectively, including variants thereof;

the V_{H}CDR1, V_{H}CDR2, and V_{H}CDR3 sequences comprise SEQ ID NOs: 102, 103, and 104, respectively, and the V_{L}CDR1, V_{L}CDR2, and V_{L}CDR3 sequences comprise SEQ ID NOs: 105, 106, and 107, respectively, including variants thereof;

the V_{H}CDR1, V_{H}CDR2, and V_{H}CDR3 sequences comprise SEQ ID NOs: 108, 109, and 110, respectively, and the V_{L}CDR1, V_{L}CDR2, and V_{L}CDR3 sequences comprise SEQ ID NOs: 111, 112, and 113, respectively, including variants thereof;

the V_{H}CDR1, V_{H}CDR2, and V_{H}CDR3 sequences comprise SEQ ID NOs: 114, 115, and 116, respectively, and the V_{L}CDR1, V_{L}CDR2, and V_{L}CDR3 sequences comprise SEQ ID NOs: 117, 118, and 119, respectively, including variants thereof;

the V_{H}CDR1, V_{H}CDR2, and V_{H}CDR3 sequences comprise SEQ ID NOs: 120, 121, and 122, respectively, and the V_{L}CDR1, V_{L}CDR2, and V_{L}CDR3 sequences comprise SEQ ID NOs: 123, 124, and 125, respectively, including variants thereof;

the V_{H}CDR1, V_{H}CDR2, and V_{H}CDR3 sequences comprise SEQ ID NOs: 126, 127, and 128, respectively, and the V_{L}CDR1, V_{L}CDR2, and V_{L}CDR3 sequences comprise SEQ ID NOs: 129, 130, and 131, respectively, including variants thereof;

the V_{H}CDR1, V_{H}CDR2, and V_{H}CDR3 sequences comprise SEQ ID NOs: 132, 133, and 134, respectively, and the V_{L}CDR1, V_{L}CDR2, and V_{L}CDR3 sequences comprise SEQ ID NOs: 135, 136, and 137, respectively, including variants thereof;

the V_{H}CDR1, V_{H}CDR2, and V_{H}CDR3 sequences comprise SEQ ID NOs: 138, 139, and 140, respectively, and the V_{L}CDR1, V_{L}CDR2, and V_{L}CDR3 sequences comprise SEQ ID NOs: 141, 142, and 143, respectively, including variants thereof;

the V_{H}CDR1, V_{H}CDR2, and V_{H}CDR3 sequences comprise SEQ ID NOs: 144, 145, and 146, respectively, and the V_{L}CDR1, V_{L}CDR2, and V_{L}CDR3 sequences comprise SEQ ID NOs: 147, 148, and 149, respectively, including variants thereof;

the V_{H}CDR1, V_{H}CDR2, and V_{H}CDR3 sequences comprise SEQ ID NOs: 150, 151, and 152, respectively, and the V_{L}CDR1, V_{L}CDR2, and V_{L}CDR3 sequences comprise SEQ ID NOs: 153, 154, and 155, respectively, including variants thereof;

the V_{H}CDR1, V_{H}CDR2, and V_{H}CDR3 sequences comprise SEQ ID NOs: 156, 157, and 158, respectively, and the V_{L}CDR1, V_{L}CDR2, and V_{L}CDR3 sequences comprise SEQ ID NOs: 159, 160, and 161, respectively, including variants thereof;

the V_{H}CDR1, V_{H}CDR2, and V_{H}CDR3 sequences comprise SEQ ID NOs: 162, 163, and 164, respectively, and the V_{L}CDR1, V_{L}CDR2, and V_{L}CDR3 sequences comprise SEQ ID NOs: 165, 166, and 167, respectively, including variants thereof;

the V_{H}CDR1, V_{H}CDR2, and V_{H}CDR3 sequences comprise SEQ ID NOs: 168, 169, and 170, respectively, and the V_{L}CDR1, V_{L}CDR2, and V_{L}CDR3 sequences comprise SEQ ID NOs: 171, 172, and 173, respectively, including variants thereof;

the V_{H}CDR1, V_{H}CDR2, and V_{H}CDR3 sequences comprise SEQ ID NOs: 174, 175, and 176, respectively, and the V_{L}CDR1, V_{L}CDR2, and V_{L}CDR3 sequences comprise SEQ ID NOs: 177, 178, and 179, respectively, including variants thereof;

the V_{H}CDR1, V_{H}CDR2, and V_{H}CDR3 sequences comprise SEQ ID NOs: 180, 181, and 182, respectively, and the V_{L}CDR1, V_{L}CDR2, and V_{L}CDR3 sequences comprise SEQ ID NOs: 183, 184, and 185, respectively, including variants thereof;

the V_{H}CDR1, V_{H}CDR2, and V_{H}CDR3 sequences comprise SEQ ID NOs: 186, 187, and 188, respectively, and the V_{L}CDR1, V_{L}CDR2, and V_{L}CDR3 sequences comprise SEQ ID NOs: 189, 190, and 191, respectively, including variants thereof;

the V_{H}CDR1, V_{H}CDR2, and V_{H}CDR3 sequences comprise SEQ ID NOs: 192, 193, and 194, respectively, and the V_{L}CDR1, V_{L}CDR2, and V_{L}CDR3 sequences comprise SEQ ID NOs: 195, 196, and 197, respectively, including variants thereof;

the V_{H}CDR1, V_{H}CDR2, and V_{H}CDR3 sequences comprise SEQ ID NOs: 198, 199, and 200, respectively, and the V_{L}CDR1, V_{L}CDR2, and V_{L}CDR3 sequences comprise SEQ ID NOs: 201, 202, and 203, respectively, including variants thereof;

the V_{H}CDR1, V_{H}CDR2, and V_{H}CDR3 sequences comprise SEQ ID NOs: 204, 205, and 206, respectively, and the V_{L}CDR1, V_{L}CDR2, and V_{L}CDR3 sequences comprise SEQ ID NOs: 207, 208, and 209, respectively, including variants thereof;

the V_{H}CDR1, V_{H}CDR2, and V_{H}CDR3 sequences comprise SEQ ID NOs: 210, 211, and 212, respectively, and the V_{L}CDR1, V_{L}CDR2, and V_{L}CDR3 sequences comprise SEQ ID NOs: 213, 214, and 215, respectively, including variants thereof;

the V_{H}CDR1, V_{H}CDR2, and V_{H}CDR3 sequences comprise SEQ ID NOs: 216, 217, and 218, respectively, and the V_{L}CDR1, V_{L}CDR2, and V_{L}CDR3 sequences comprise SEQ ID NOs: 219, 220, and 221, respectively, including variants thereof;

the V_{H}CDR1, V_{H}CDR2, and V_{H}CDR3 sequences comprise SEQ ID NOs: 222, 223, and 224, respectively, and the V_{L}CDR1, V_{L}CDR2, and V_{L}CDR3 sequences comprise SEQ ID NOs: 225, 226, and 227, respectively, including variants thereof;

the V_{H}CDR1, V_{H}CDR2, and V_{H}CDR3 sequences comprise SEQ ID NOs: 228, 229, and 230, respectively, and the V_{L}CDR1, V_{L}CDR2, and V_{L}CDR3 sequences comprise SEQ ID NOs: 231, 232, and 233, respectively, including variants thereof;

the V_{H}CDR1, V_{H}CDR2, and V_{H}CDR3 sequences comprise SEQ ID NOs: 234, 235, and 236, respectively, and the V_{L}CDR1, V_{L}CDR2, and V_{L}CDR3 sequences comprise SEQ ID NOs: 237, 238, and 239, respectively, including variants thereof;

the V_{H}CDR1, V_{H}CDR2, and V_{H}CDR3 sequences comprise SEQ ID NOs: 240, 241, and 242, respectively, and the V_{L}CDR1, V_{L}CDR2, and V_{L}CDR3 sequences comprise SEQ ID NOs: 243, 244, and 245, respectively, including variants thereof;

the V_{H}CDR1, V_{H}CDR2, and V_{H}CDR3 sequences comprise SEQ ID NOs: 246, 247, and 248, respectively, and the V_{L}CDR1, V_{L}CDR2, and V_{L}CDR3 sequences comprise SEQ ID NOs: 249, 250, and 251, respectively, including variants thereof;

the V_{H}CDR1, V_{H}CDR2, and V_{H}CDR3 sequences comprise SEQ ID NOs: 252, 253, and 254, respectively, and the V_{L}CDR1, V_{L}CDR2, and V_{L}CDR3 sequences comprise SEQ ID NOs: 255, 256, and 257, respectively, including variants thereof;

the V_{H}CDR1, V_{H}CDR2, and V_{H}CDR3 sequences comprise SEQ ID NOs: 258, 259, and 260, respectively, and the V_{L}CDR1, V_{L}CDR2, and V_{L}CDR3 sequences comprise SEQ ID NOs: 261, 262, and 263, respectively, including variants thereof;

the V_{H}CDR1, V_{H}CDR2, and V_{H}CDR3 sequences comprise SEQ ID NOs: 264, 265, and 266, respectively, and the V_{L}CDR1, V_{L}CDR2, and V_{L}CDR3 sequences comprise SEQ ID NOs: 267, 268, and 269, respectively, including variants thereof;

the V_{H}CDR1, V_{H}CDR2, and V_{H}CDR3 sequences comprise SEQ ID NOs: 270, 271, and 272, respectively, and the V_{L}CDR1, V_{L}CDR2, and V_{L}CDR3 sequences comprise SEQ ID NOs: 273, 274, and 275, respectively, including variants thereof;

the V_{H}CDR1, V_{H}CDR2, and V_{H}CDR3 sequences comprise SEQ ID NOs: 276, 277, and 278, respectively, and the V_{L}CDR1, V_{L}CDR2, and V_{L}CDR3 sequences comprise SEQ ID NOs: 279, 280, and 281, respectively, including variants thereof;

the V_{H}CDR1, V_{H}CDR2, and V_{H}CDR3 sequences comprise SEQ ID NOs: 282, 283, and 284, respectively, and the V_{L}CDR1, V_{L}CDR2, and V_{L}CDR3 sequences comprise SEQ ID NOs: 285, 286, and 287, respectively, including variants thereof;

the V_{H}CDR1, V_{H}CDR2, and V_{H}CDR3 sequences comprise SEQ ID NOs: 288, 289, and 290, respectively, and the V_{L}CDR1, V_{L}CDR2, and V_{L}CDR3 sequences comprise SEQ ID NOs: 291, 292, and 293, respectively, including variants thereof;

the V_{H}CDR1, V_{H}CDR2, and V_{H}CDR3 sequences comprise SEQ ID NOs: 294, 295, and 296, respectively, and the V_{L}CDR1, V_{L}CDR2, and V_{L}CDR3 sequences comprise SEQ ID NOs: 297, 298, and 299, respectively, including variants thereof; and/or

the V_{H}CDR1, V_{H}CDR2, and V_{H}CDR3 sequences comprise SEQ ID NOs: 300, 301, and 302, respectively, and the V_{L}CDR1, V_{L}CDR2, and V_{L}CDR3 sequences comprise SEQ ID NOs: 303, 304, and 305, respectively, including variants thereof.

In certain embodiments, the V_{H} sequence is at least 80, 85, 90, 95, 97, 98, 99, or 100% identical to a sequence selected from Table A2, including, for example, wherein the V_{H} sequence has 1, 2, 3, 4, or 5 alterations in one or more framework regions.

In some embodiments, the V_{L} sequence is at least 80, 85, 90, 95, 97, 98, 99, or 100% identical to a sequence selected from Table A2, including, for example, wherein the V_{L} sequence has 1, 2, 3, 4, or 5 alterations in one or more framework regions.

In some embodiments, the V_{H} and V_{L} sequences of an antibody or antigen-binding fragment are as follows:

the V_{H} sequence comprises SEQ ID NO:30, and the V_{L} sequence comprises SEQ ID NO:31;

the V_{H} sequence comprises SEQ ID NO:32, and the V_{L} sequence comprises SEQ ID NO:33;

the V_{H} sequence comprises SEQ ID NO:34, and the V_{L} sequence comprises SEQ ID NO:35;

the V_{H} sequence comprises SEQ ID NO:306, and the V_{L} sequence comprises SEQ ID NO:307;

the V_{H} sequence comprises SEQ ID NO:308, and the V_{L} sequence comprises SEQ ID NO:309;

the V_{H} sequence comprises SEQ ID NO:310, and the V_{L} sequence comprises SEQ ID NO:311;

the V_{H} sequence comprises SEQ ID NO:312, and the V_{L} sequence comprises SEQ ID NO:313;

the V_{H} sequence comprises SEQ ID NO:314, and the V_{L} sequence comprises SEQ ID NO:315;

the V_{H} sequence comprises SEQ ID NO:316, and the V_{L} sequence comprises SEQ ID NO:317;

the V_{H} sequence comprises SEQ ID NO:318, and the V_{L} sequence comprises SEQ ID NO:319;

the V_{H} sequence comprises SEQ ID NO:320, and the V_{L} sequence comprises SEQ ID NO:321;

the V_{H} sequence comprises SEQ ID NO:322, and the V_{L} sequence comprises SEQ ID NO:323;

the V_{H} sequence comprises SEQ ID NO:324, and the V_{L} sequence comprises SEQ ID NO:325;

the V_{H} sequence comprises SEQ ID NO:326, and the V_{L} sequence comprises SEQ ID NO:327;

the V_{H} sequence comprises SEQ ID NO:328, and the V_{L} sequence comprises SEQ ID NO:329;

the V_{H} sequence comprises SEQ ID NO:330, and the V_{L} sequence comprises SEQ ID NO:331;

the V_{H} sequence comprises SEQ ID NO:332, and the V_{L} sequence comprises SEQ ID NO:333;

the V_{H} sequence comprises SEQ ID NO:334, and the V_{L} sequence comprises SEQ ID NO:335;

the V_{H} sequence comprises SEQ ID NO:336, and the V_{L} sequence comprises SEQ ID NO:337;

the V_{H} sequence comprises SEQ ID NO:338, and the V_{L} sequence comprises SEQ ID NO:339;

the V_{H} sequence comprises SEQ ID NO:340, and the V_{L} sequence comprises SEQ ID NO:341;

the V_{H} sequence comprises SEQ ID NO:342, and the V_{L} sequence comprises SEQ ID NO:343;

the V_{H} sequence comprises SEQ ID NO:344, and the V_{L} sequence comprises SEQ ID NO:345;

the V_{H} sequence comprises SEQ ID NO:346, and the V_{L} sequence comprises SEQ ID NO:347;

the V_{H} sequence comprises SEQ ID NO:348, and the V_{L} sequence comprises SEQ ID NO:349;

the V_{H} sequence comprises SEQ ID NO:350, and the V_{L} sequence comprises SEQ ID NO:351;

the V_{H} sequence comprises SEQ ID NO:352, and the V_{L} sequence comprises SEQ ID NO:353;

the V_{H} sequence comprises SEQ ID NO:354, and the V_{L} sequence comprises SEQ ID NO:355;

the V_{H} sequence comprises SEQ ID NO:356, and the V_{L} sequence comprises SEQ ID NO:357;

the V_{H} sequence comprises SEQ ID NO:358, and the V_{L} sequence comprises SEQ ID NO:359;

the V_{H} sequence comprises SEQ ID NO:360, and the V_{L} sequence comprises SEQ ID NO:361;

the V_{H} sequence comprises SEQ ID NO:362, and the V_{L} sequence comprises SEQ ID NO:363;

the V_{H} sequence comprises SEQ ID NO:364, and the V_{L} sequence comprises SEQ ID NO:365;

the V_{H} sequence comprises SEQ ID NO:366, and the V_{L} sequence comprises SEQ ID NO:367;

the V_{H} sequence comprises SEQ ID NO:368, and the V_{L} sequence comprises SEQ ID NO:369;

the V_{H} sequence comprises SEQ ID NO:370, and the V_{L} sequence comprises SEQ ID NO:371;

the V_{H} sequence comprises SEQ ID NO:372, and the V_{L} sequence comprises SEQ ID NO:373;

the V_{H} sequence comprises SEQ ID NO:374, and the V_{L} sequence comprises SEQ ID NO:375;

the V_{H} sequence comprises SEQ ID NO:376, and the V_{L} sequence comprises SEQ ID NO:377;

the V_{H} sequence comprises SEQ ID NO:378, and the V_{L} sequence comprises SEQ ID NO:379;

the V_{H} sequence comprises SEQ ID NO:380, and the V_{L} sequence comprises SEQ ID NO:381;

the V_{H} sequence comprises SEQ ID NO:382, and the V_{L} sequence comprises SEQ ID NO:383;

the V_{H} sequence comprises SEQ ID NO:384, and the V_{L} sequence comprises SEQ ID NO:385;

the V_{H} sequence comprises SEQ ID NO:386, and the V_{L} sequence comprises SEQ ID NO:387;

the V_{H} sequence comprises SEQ ID NO:388, and the V_{L} sequence comprises SEQ ID NO:389;

the V_{H} sequence comprises SEQ ID NO:390, and the V_{L} sequence comprises SEQ ID NO:391;

the V_{H} sequence comprises SEQ ID NO:392, and the V_{L} sequence comprises SEQ ID NO:393; and/or

the V_{H} sequence comprises SEQ ID NO:394, and the V_{L} sequence comprises SEQ ID NO:395.

Also included are variants thereof, for example, variants having 1, 2, 3, 4, or 5 alterations in one or more framework regions. Exemplary “alterations” include amino acid substitutions, additions, and deletions.

In some embodiments, an antibody or antigen-binding fragment thereof is derived or obtained from a human or other animal source which naturally-produces anti-HRS antibodies. For instance, certain subjects with polymyositis and/or dermatomyositis are known to naturally develop antibodies to the Jo-1 antigen, which has been established to comprise full-length HRS (see, for example, Targoff, Current Opinion in Rheumatology. 12:475-481, 2000). Thus, certain embodiments include one or more naturally-occurring anti-HRS antibodies (or “anti-Jo-1 antibodies”) or antigen-binding fragments thereof. “Anti-Jo-1 antibodies” are myositis specific autoantibodies most commonly found in patients with idiopathic inflammatory myopathies (IIM) such as polymyositis and/or dermatomyositis, and are directed against human HRS. In some embodiments, an antibody or antigen-binding fragment thereof is derived or obtained from a donor subject, for example, a donor subject with an IIM such as polymyositis and/or dermatomyositis. In particular embodiments, the naturally-occurring antibodies anti-Jo-1 antibodies are obtained from plasma or serum of the donor subject(s), for example, human donor subject(s). In some embodiments, the one or more human donor subjects have an anti-Jo-1 antibody serum content or level of about or at least about 0.1 μg/mL, 0.2 μg/mL, 0.5 μg/mL, 1 μg/mL, 2 μg/mL, 5 μg/mL, 10 μg/mL, 20 μg/mL, 50 μg/mL, or 100 μg/mL. In certain embodiments, a naturally-occurring antibody, or antigen-binding fragment thereof, has an affinity (Kd) for an HRS polypeptide or epitope described herein (see Table H1 or Table H2) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In some embodiments, the epitope is in the N-terminal region of HRS, for example, wherein the epitope is within about residues 1-100, 1-90, 1-80, 1-70, 1-60, 1-50, or 1-40 of SEQ ID NO:1 (FL human HRS). In certain embodiments, an antibody or antigen-binding fragment thereof specifically binds to the aminoacylation domain region of the HRS polypeptide (e.g., binds to an epitope within amino acids 61-398 of full length HRS). In certain embodiments, an antibody or antigen-binding fragment thereof specifically binds to the anti-codon binding domain region of the HRS polypeptide (e.g., binds to an epitope within amino acids 399-509 of full length HRS). In certain embodiments, an antibody or antigen-binding fragment thereof specifically binds to a HRS splice variant of Table H1. In certain embodiments, an antibody or antigen-binding fragment thereof specifically binds to a HRS splice variant selected from SV9 (HRS(1-60)), SV11(HRS(1-60)+(399-509)) and SV14(HRS(1-100)+(399-509)). In certain embodiments, an antibody or antigen-binding fragment thereof selectively binds only to a monomeric form of the HRS polypeptide, and does not substantially bind to a dimeric or multimeric form of the HRS polypeptide.

In some embodiments, anti-HRS antibodies or antigen-binding fragments thereof are composed of a polyclonal mixture of antibodies. In some embodiments, as above, the polyclonal mixture of anti-HRS antibodies is composed of naturally-occurring anti-Jo-1 antibodies obtained from the plasma or serum of one or more donor subjects, for example, human antibodies obtained from human donor subject(s). In some embodiments, the subject(s) have an anti-Jo-1 antibody serum level of about or at least about 0.1 μg/mL, 0.2 μg/mL, 0.5 μg/mL, 1 μg/mL, 2 μg/mL, 5 μg/mL, 10 μg/mL, 20 μg/mL, 50 μg/mL, or 100 μg/mL. In some instances, the polyclonal mixture of antibodies is a serum or plasma preparation that is substantially-free of other serum immunoglobulins. In some embodiments, the polyclonal mixture of antibodies is a serum or plasma preparation that comprises other serum immunoglobulins. In certain embodiments, a polyclonal mixture of antibodies has an average affinity (Kd) for an HRS polypeptide or epitope described herein (see Table H1 or Table H2) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM. In some embodiments, the epitope is within the N-terminal region of HRS, for example, wherein the epitope is within about residues 1-100, 1-90, 1-80, 1-70, 1-60, 1-50, or 1-40 of SEQ ID NO: 1 (FL human HRS). In certain embodiments, the epitope is within the aminoacylation domain region of the HRS polypeptide (e.g., within amino acids 61-398 of full length HRS). In certain embodiments, the epitope is within the anti-codon binding domain region of the HRS polypeptide (e.g., within amino acids 399-509 of full length HRS). In certain embodiments, the epitope is within a HRS splice variant of Table H1. In certain embodiments, the epitope is within a HRS splice variant selected from SV9 (HRS(1-60)), SV11(HRS(1-60)+(399-509)) and SV14(HRS(1-100)+(399-509)). In certain embodiments, the epitope is selectively exposed in a monomeric form of the HRS polypeptide, and is not substantially exposed in a dimeric or multimeric form of the HRS polypeptide.

In some embodiments, a polyclonal mixture of antibodies is enriched for antibodies of a particular Ig class, for example, antibodies of the IgG, IgM, IgE, or IgA classes, or any combination thereof. In certain embodiments, the IgG class of the polyclonal mixture of antibodies is enriched for one or more IgG subclasses, for example, one or more of the IgG1, IgG2, IgG3, or IgG4 subclasses, or any combination thereof. In certain embodiments, the polyclonal mixture of antibodies is enriched for antibodies of the IgG class, relative to antibodies of the IgM, IgE or IgA classes. Thus, in some embodiments, in the least because of the enrichment process and/or because of the combination of antibodies from more than one donor subject, the polyclonal mixture of antibodies is not a naturally-occurring mixture. Preparative steps can be used to enrich a particular isotype or subtype of immunoglobulin. For example, protein A, protein G, or protein H sepharose chromatography can be used to enrich a mixture of immunoglobulins for IgG, or for specific IgG subtypes. (See generally Harlow and Lane, Using Antibodies, Cold Spring Harbor Laboratory Press (1999); Harlow and Lane, Antibodies, A Laboratory Manual, Cold Spring Harbor Laboratory Press (1988); U.S. Pat. No. 5,180,810).

Commercial sources of immunoglobulins can also be used to prepare the one or more naturally-occurring anti-HRS antibodies, or antigen-binding fragments thereof, or polyclonal mixtures of antibodies, for example, enriched polyclonal mixtures of antibodies. Such sources include but are not limited to: Gammagard S/D® (Baxter Healthcare); BayRho-D® products (Bayer Biological); Gamimune N®, 5% (Bayer Biological); Gamimune N®, 5% Solvent/Detergent Treated (Bayer Biological); Gamimune N®, 10% (Bayer Biological); Sandoglobulin I.V.® (Novartis); Polygam S/D® (American Red Cross); Venoglobulin-S® 5% Solution Solvent Detergent Treated (Alpha Therapeutic); Venoglobulin-S® 10% Solution Solvent Detergent/Treated (Alpha Therapeutic); and VZIG® (American Red Cross). The commercial source of the immunoglobulin preparation is not critical, provided that the donor subjects are pre-screened for Jo-1 positivity.

In certain embodiments, an antibody or antigen-binding fragment thereof comprises variant or otherwise modified Fc region(s), including those having altered properties or biological activities relative to wild-type Fc region(s). Examples of modified Fc regions include those having mutated sequences, for instance, by substitution, insertion, deletion, or truncation of one or more amino acids relative to a wild-type sequence, hybrid Fc polypeptides composed of domains from different immunoglobulin classes/subclasses, Fc polypeptides having altered glycosylation/sialylation patterns, and Fc polypeptides that are modified or derivatized, for example, by biotinylation (see, e.g., US Application No. 2010/0209424), phosphorylation, sulfation, etc., or any combination of the foregoing. Such modifications can be employed to alter (e.g., increase, decrease) the binding properties of the Fc region to one or more particular FcRs (e.g., FcγRI, FcγRIIa, FcγRIIb, FcγRIIc, FcγRIIIa, FcγRIIIb, FcRn), its pharmacokinetic properties (e.g., stability or half-life, bioavailability, tissue distribution, volume of distribution, concentration, elimination rate constant, elimination rate, area under the curve (AUC), clearance, Cₘₐₓ, tₘₐₓ, Cₘᵢₙ, fluctuation), its immunogenicity, its complement fixation or activation, and/or the CDC/ADCC/ADCP-related activities of the Fc region, among other properties described herein, relative to a corresponding wild-type Fc sequence of an antibody or antigen-binding fragment thereof. Included are modified Fc regions of human and/or mouse origin.

Also included are antibodies or antigen-binding fragments thereof that comprise hybrid Fc regions, for example, Fc regions that comprise a combination of Fc domains (e.g., hinge, CH₂, CH₃, CH₄) from immunoglobulins of different species (e.g., human, mouse), different Ig classes, and/or different Ig subclasses. General examples include hybrid Fc regions that comprise, consist of, or consist essentially of the following combination of CH₂/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgE/IgA1, IgE/IgA2, IgE/IgD, IgE/IgE, IgE/IgG1, IgE/IgG2, IgE/IgG3, IgE/IgG4, IgE/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM, IgM/IgA1, IgM/IgA2, IgM/IgD, IgM/IgE, IgM/IgG1, IgM/IgG2, IgM/IgG3, IgM/IgG4, IgM/IgM (or fragments or variants thereof), and optionally include a hinge from one or more of IgA1, IgA2, IgD, IgG1, IgG2, IgG3, or IgG4, and/or a CH₄ domain from IgE and/or IgM. In specific embodiments, the hinge, CH₂, CH₃, and CH₄ domains are from human Ig.

Additional examples include hybrid Fc regions that comprise, consist of, or consist essentially of the following combination of CH₂/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₃ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Certain examples include hybrid Fc regions that comprise, consist of, or consist essentially of the following combination of CH₃/CH₄ domains: IgA1/IgE, IgA2/IgE, IgD/IgE, IgE/IgE, IgG1/IgE, IgG2/IgE, IgG3/IgE, IgG4/IgE, IgM/IgE, IgA1/IgM, IgA2/IgM, IgD/IgM, IgE/IgM, IgG1/IgM, IgG2/IgM, IgG3/IgM, IgG4/IgM, IgM/IgM (or fragments or variants thereof), and optionally include a hinge from one or more of IgA1, IgA2, IgD, IgG1, IgG2, IgG3, IgG4, and/or a CH₂ domain from one or more of IgA1, IgA2, IgD, IgE, IgG1, IgG2, IgG3, IgG4, or IgM. In specific embodiments, the hinge, CH₂, CH₃, and CH₄ domains are from human Ig.

Particular examples include hybrid Fc regions that comprise, consist of, or consist essentially of the following combination of hinge/CH₂ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₃ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Certain examples include hybrid Fc regions that comprise, consist of, or consist essentially of the following combination of hinge/CH₃ domains: IgA1/IgA1, IgA1/IgA2, IgA1/IgD, IgA1/IgE, IgA1/IgG1, IgA1/IgG2, IgA1/IgG3, IgA1/IgG4, IgA1/IgM, IgA2/IgA1, IgA2/IgA2, IgA2/IgD, IgA2/IgE, IgA2/IgG1, IgA2/IgG2, IgA2/IgG3, IgA2/IgG4, IgA2/IgM, IgD/IgA1, IgD/IgA2, IgD/IgD, IgD/IgE, IgD/IgG1, IgD/IgG2, IgD/IgG3, IgD/IgG4, IgD/IgM, IgG1/IgA1, IgG1/IgA2, IgG1/IgD, IgG1/IgE, IgG1/IgG1, IgG1/IgG2, IgG1/IgG3, IgG1/IgG4, IgG1/IgM, IgG2/IgA1, IgG2/IgA2, IgG2/IgD, IgG2/IgE, IgG2/IgG1, IgG2/IgG2, IgG2/IgG3, IgG2/IgG4, IgG2/IgM, IgG3/IgA1, IgG3/IgA2, IgG3/IgD, IgG3/IgE, IgG3/IgG1, IgG3/IgG2, IgG3/IgG3, IgG3/IgG4, IgG3/IgM, IgG4/IgA1, IgG4/IgA2, IgG4/IgD, IgG4/IgE, IgG4/IgG1, IgG4/IgG2, IgG4/IgG3, IgG4/IgG4, IgG4/IgM (or fragments or variants thereof), and optionally include a CH₂ domain from one or more of IgA1, IgA2, IgD, IgE, IgG1, IgG2, IgG3, IgG4, or IgM, and/or a CH₄ domain from IgE and/or IgM. In specific embodiments, the hinge, CH₂, CH₃, and CH₄ domains are from human Ig.

Some examples include hybrid Fc regions that comprise, consist of, or consist essentially of the following combination of hinge/CH₄ domains: IgA1/IgE, IgA1/IgM, IgA2/IgE, IgA2/IgM, IgD/IgE, IgD/IgM, IgG1/IgE, IgG1/IgM, IgG2/IgE, IgG2/IgM, IgG3/IgE, IgG3/IgM, IgG4/IgE, IgG4/IgM (or fragments or variants thereof), and optionally include a CH₂ domain from one or more of IgA1, IgA2, IgD, IgE, IgG1, IgG2, IgG3, IgG4, or IgM, and/or a CH₃ domain from one or more of IgA1, IgA2, IgD, IgE, IgG1, IgG2, IgG3, IgG4, or IgM.

Specific examples of hybrid Fc regions can be found, for example, in WO 2008/147143, which are derived from combinations of IgG subclasses or combinations of human IgD and IgG.

Also included are antibodies or antigen-binding fragments thereof having derivatized or otherwise modified Fc regions. In certain aspects, the Fc region may be modified by phosphorylation, sulfation, acrylation, glycosylation, methylation, farnesylation, acetylation, amidation, and the like, for instance, relative to a wild-type or naturally-occurring Fc region. In certain embodiments, the Fc region may comprise wild-type or native glycosylation patterns, or alternatively, it may comprise increased glycosylation relative to a native form, decreased glycosylation relative to a native form, or it may be entirely deglycosylated. As one example of a modified Fc glycoform, decreased glycosylation of an Fc region reduces binding to the C1q region of the first complement component C1, a decrease in ADCC-related activity, and/or a decrease in CDC-related activity. Certain embodiments thus employ a deglycosylated or aglycosylated Fc region. See, e.g., WO 2005/047337 for the production of exemplary aglycosylated Fc regions. Another example of an Fc region glycoform can be generated by substituting the Q295 position with a cysteine residue (see, e.g., U.S. Application No. 2010/0080794), according to the Kabat et al. numbering system. Certain embodiments may include Fc regions where about 80-100% of the glycoprotein in Fc region comprises a mature core carbohydrate structure that lacks fructose (see, e.g., U.S. Application No. 2010/0255013). Some embodiments may include Fc regions that are optimized by substitution or deletion to reduce the level of fucosylation, for instance, to increase affinity for FcγRI, FcγRIa, or FcγRIIIa, and/or to improve phagocytosis by FcγRIIa-expressing cells (see U.S. Application Nos. 2010/0249382 and 2007/0148170).

As another example of a modified Fc glycoform, an Fc region of an antibody or antigen-binding fragment thereof may comprise oligomannose-type N-glycans, and optionally have one or more of the following: increased ADCC effector activity, increased binding affinity for FcγRIIIA (and certain other FcRs), similar or increased binding specificity for the target of the HRS polypeptide, similar or higher binding affinity for the target of the HRS polypeptide, and/or similar or lower binding affinity for mannose receptor, relative to a corresponding Fc region that contains complex-type N-glycans (see, e.g., U.S. Application No. 2007/0092521 and U.S. Pat. No. 7,700,321). As another example, enhanced affinity of Fc regions for FcγRs has been achieved using engineered glycoforms generated by expression of antibodies in engineered or variant cell lines (see, e.g., Umana et al., Nat Biotechnol. 17:176-180, 1999; Davies et al., Biotechnol Bioeng. 74:288-294, 2001; Shields et al., J. Biol Chem. 277:26733-26740, 2002; Shinkawa et al., 2003, J Biol Chem. 278:3466-3473, 2003; and U.S. Application No. 2007/0111281). Certain Fc region glycoforms comprise an increased proportion of N-glycoside bond type complex sugar chains, which do not have the 1-position of fucose bound to the 6-position of N-acetylglucosamine at the reducing end of the sugar chain (see, e.g., U.S. Application No. 2010/0092997). Particular embodiments may include IgG Fc region that is glycosylated with at least one galactose moiety connected to a respective terminal sialic acid moiety by an α-2,6 linkage, optionally where the Fc region has a higher anti-inflammatory activity relative to a corresponding, wild-type Fc region (see U.S. Application No. 2008/0206246). Certain of these and related altered glycosylation approaches have generated substantial enhancements of the capacity of Fc regions to selectively bind FcRs such as FcγRIII, to mediate ADCC, and to alter other properties of Fc regions, as described herein.

Certain variant, fragment, hybrid, or otherwise modified Fc regions of an antibody or antigen-binding fragment thereof may have altered binding to one or more FcRs, and/or corresponding changes to effector function, relative to a corresponding, wild-type Fc sequence (e.g., same species, same Ig class, same Ig subclass). For instance, such Fc regions may have increased binding to one or more of Fcγ receptors, Fcα receptors, Fcε receptors, and/or the neonatal Fc receptor, relative to a corresponding, wild-type Fc sequence. In other embodiments, variant, fragment, hybrid, or modified Fc regions may have decreased binding to one or more of Fcγ receptors, Fcα receptors, Fcε receptors, and/or the neonatal Fc receptor, relative to a corresponding, wild-type Fc sequence. Specific FcRs are described elsewhere herein.

In some embodiments, an antibody comprises an Fc domain, comprising one or more mutations to increase binding to one or more of Fcγ receptors, Fcα receptors, Fcε receptors, and/or the neonatal Fc receptor, relative to a corresponding, wild-type Fc sequence. In some embodiments, an antibody comprises an IgG1 or IgG3 Fc domain, comprising one or more mutations to increase binding to one or more of Fcγ receptors, Fcα receptors, Fcε receptors, and/or the neonatal Fc receptor, relative to a corresponding, wild-type Fc sequence. In some embodiments, an antibody comprises an Fc domain, comprising one or more mutations to increase effector function. In some embodiments the at least one antibody comprises an Fc domain selected from a human IgG1 and IgG3, comprising one or more mutations to increase effector function.

In some embodiments, an antibody is blocking antibody that comprises an Fc domain with high effector activity. In some embodiments, the blocking antibody comprises an Fc domain selected from a human IgG1 and IgG3, comprising one or more mutations to increase effector function. In some embodiments, an antibody is a partial-blocking antibody that comprises an Fc domain with high effector activity. In some embodiments, the a partial-blocking antibody comprises an Fc domain selected from a human IgG1 and IgG3, comprising one or more mutations to increase effector function. In some embodiments, an antibody is a non-blocking antibody that comprises an Fc domain with high effector activity. In some embodiments, the non-blocking antibody comprises an Fc domain selected from a human IgG1 or IgG3, comprising one or more mutations to increase effector function.

In some embodiments, an antibody comprises an Fc domain, comprising one or more mutations to decrease binding to one or more of Fcγ receptors, Fcα receptors, Fcε receptors, and/or the neonatal Fc receptor, relative to a corresponding, wild-type Fc sequence. In some embodiments, an antibody comprises an IgG1 or IgG3 Fc domain, comprising one or more mutations to decrease binding to one or more of Fcγ receptors, Fcα receptors, Fcε receptors, and/or the neonatal Fc receptor, relative to a corresponding, wild-type Fc sequence. In some embodiments, an antibody comprises an Fc domain, comprising one or more mutations to decrease effector function. In some embodiments, an antibody comprises an Fc domain selected from a human IgG2 and IgG4, comprising one or more mutations to decrease effector function.

In some embodiments, an antibody is a blocking antibody comprising an Fc domain with low effector activity. In some embodiments, the blocking antibody comprises an Fc domain selected from a human IgG2 and IgG4, comprising one or more mutations to decrease effector function. In some embodiments, an antibody is a partial-blocking antibody comprising an Fc domain with low effector activity. In some embodiments, the partial-blocking antibody comprises an Fc domain selected from a human IgG2 and IgG4, comprising one or more mutations to decrease effector function. In some embodiments, an antibody is a non-blocking antibody comprising an Fc domain with low effector activity. In some embodiments, the non-blocking antibody comprises an Fc domain selected from a human IgG2 and IgG4, comprising one or more mutations to decrease effector function.

Specific examples of Fc variants having altered (e.g., increased, decreased) effector function/FcR binding can be found, for example, in U.S. Pat. Nos. 5,624,821 and 7,425,619; U.S. Application Nos. 2009/0017023, 2009/0010921, and 2010/0203046; and WO 2000/42072 and WO 2004/016750. Certain examples include human Fc regions having a one or more substitutions at position 298, 333, and/or 334, for example, S298A, E333A, and/or K334A (based on the numbering of the EU index of Kabat et al.), which have been shown to increase binding to the activating receptor FcγRIIIa and reduce binding to the inhibitory receptor FcγRIIb. These mutations can be combined to obtain double and triple mutation variants that have further improvements in binding to FcRs. Certain embodiments include a S298A/E333A/K334A triple mutant, which has increased binding to FcγRIIIa, decreased binding to FcγRIIb, and increased ADCC (see, e.g., Shields et al., J Biol Chem. 276:6591-6604, 2001; and Presta et al., Biochem Soc Trans. 30:487-490, 2002). See also engineered Fc glycoforms that have increased binding to FcRs, as disclosed in Umana et al., supra; and U.S. Pat. No. 7,662,925. Some embodiments include Fc regions that comprise one or more substitutions selected from 434S, 252Y/428L, 252Y/434S, and 428L/434S (see U.S. Application Nos. 2009/0163699 and 20060173170), based on the EU index of Kabat et al.

Certain variant, fragment, hybrid, or modified Fc regions may have altered effector functions, relative to a corresponding, wild-type Fc sequence. For example, such Fc regions may have increased complement fixation or activation, increased Clq binding affinity, increased CDC-related activity, increased ADCC-related activity, and/or increased ADCP-related activity, relative to a corresponding, wild-type Fc sequence. In other embodiments, such Fc regions may have decreased complement fixation or activation, decreased Clq binding affinity, decreased CDC-related activity, decreased ADCC-related activity, and/or decreased ADCP-related activity, relative to a corresponding, wild-type Fc sequence. As merely one illustrative example, an Fc region may comprise a deletion or substitution in a complement-binding site, such as a C1q-binding site, and/or a deletion or substitution in an ADCC site. Examples of such deletions/substitutions are described, for example, in U.S. Pat. No. 7,030,226. Many Fc effector functions, such as ADCC, can be assayed according to routine techniques in the art. (see, e.g., Zuckerman et al., CRC Crit Rev Microbiol. 7:1-26, 1978). Useful effector cells for such assays includes, but are not limited to, natural killer (NK) cells, macrophages, and other peripheral blood mononuclear cells (PBMC). Alternatively, or additionally, certain Fc effector functions may be assessed in vivo, for example, by employing an animal model described in Clynes et al. PNAS. 95:652-656, 1998.

Certain variant hybrid, or modified Fc regions may have altered stability or half-life relative to a corresponding, wild-type Fc sequence. In certain embodiments, such Fc regions may have increased half-life relative to a corresponding, wild-type Fc sequence. In other embodiments, variant hybrid, or modified Fc regions may have decreased half-life relative to a corresponding, wild-type Fc sequence. Half-life can be measured in vitro (e.g., under physiological conditions) or in vivo, according to routine techniques in the art, such as radiolabeling, ELISA, or other methods. In vivo measurements of stability or half-life can be measured in one or more bodily fluids, including blood, serum, plasma, urine, or cerebrospinal fluid, or a given tissue, such as the liver, kidneys, muscle, central nervous system tissues, bone, etc. As one example, modifications to an Fc region that alter its ability to bind the FcRn can alter its half-life in vivo. Assays for measuring the in vivo pharmacokinetic properties (e.g., in vivo mean elimination half-life) and non-limiting examples of Fc modifications that alter its binding to the FcRn are described, for example, in U.S. Pat. Nos. 7,217,797 and 7,732,570; and U.S. Application Nos. US 2010/0143254 and 2010/0143254.

Additional non-limiting examples of modifications to alter stability or half-life include substitutions/deletions at one or more of amino acid residues selected from 251-256, 285-290, and 308-314 in the CH₂ domain, and 385-389 and 428-436 in the CH₃ domain, according to the numbering system of Kabat et al. See U.S. Application No. 2003/0190311. Specific examples include substitution with leucine at position 251, substitution with tyrosine, tryptophan or phenylalanine at position 252, substitution with threonine or serine at position 254, substitution with arginine at position 255, substitution with glutamine, arginine, serine, threonine, or glutamate at position 256, substitution with threonine at position 308, substitution with proline at position 309, substitution with serine at position 311, substitution with aspartate at position 312, substitution with leucine at position 314, substitution with arginine, aspartate or serine at position 385, substitution with threonine or proline at position 386, substitution with arginine or proline at position 387, substitution with proline, asparagine or serine at position 389, substitution with methionine or threonine at position 428, substitution with tyrosine or phenylalanine at position 434, substitution with histidine, arginine, lysine or serine at position 433, and/or substitution with histidine, tyrosine, arginine or threonine at position 436, including any combination thereof. Such modifications optionally increase affinity of the Fc region for the FcRn and thereby increase half-life, relative to a corresponding, wild-type Fc region.

Certain variant hybrid, or modified Fc regions may have altered solubility relative to a corresponding, wild-type Fc sequence. In certain embodiments, such Fc regions may have increased solubility relative to a corresponding, wild-type Fc sequence. In other embodiments, variant hybrid, or modified Fc regions may have decreased solubility relative to a corresponding, wild-type Fc sequence. Solubility can be measured, for example, in vitro (e.g., under physiological conditions) according to routine techniques in the art. Exemplary solubility measurements are described elsewhere herein.

Additional examples of variants include IgG Fc regions having conservative or non-conservative substitutions (as described elsewhere herein) at one or more of positions 250, 314, or 428 of the heavy chain, or in any combination thereof, such as at positions 250 and 428, or at positions 250 and 314, or at positions 314 and 428, or at positions 250, 314, and 428 (see, e.g., U.S. Application No. 2011/0183412). In specific embodiments, the residue at position 250 is substituted with glutamic acid or glutamine, and/or the residue at position 428 is substituted with leucine or phenylalanine. As another illustrative example of an IgG Fc variant, any one or more of the amino acid residues at positions 214 to 238, 297 to 299, 318 to 322, and/or 327 to 331 may be used as a suitable target for modification (e.g., conservative or non-conservative substitution, deletion). In particular embodiments, the IgG Fc variant CH₂ domain contains amino acid substitutions at positions 228, 234, 235, and/or 331 (e.g., human IgG4 with Ser228Pro and Leu235Ala mutations) to attenuate the effector functions of the Fc region (see U.S. Pat. No. 7,030,226). Here, the numbering of the residues in the heavy chain is that of the EU index (see Kabat et al., “Sequences of Proteins of Immunological Interest,” 5^{th} Ed., National Institutes of Health, Bethesda, Md. (1991)). Certain of these and related embodiments have altered (e.g., increased, decreased) FcRn binding and/or serum half-life, optionally without reduced effector functions such as ADCC or CDC-related activities.

Additional examples include variant Fc regions that comprise one or more amino acid substitutions at positions 279, 341, 343 or 373 of a wild-type Fc region, or any combination thereof (see, e.g., U.S. Application No. 2007/0224188). The wild-type amino acid residues at these positions for human IgG are valine (279), glycine (341), proline (343) and tyrosine (373). The substation(s) can be conservative or non-conservative, or can include non-naturally occurring amino acids or mimetics, as described herein. Alone or in combination with these substitutions, certain embodiments may also employ a variant Fc region that comprises at least 1, 2, 3, 4, 5, 6, 7, 8, 9, 10 or more amino acid substitutions selected from the following: 235G, 235R, 236F, 236R, 236Y, 237K, 237N, 237R, 238E, 238G, 238H, 238I, 238L, 238V, 238W, 238Y, 244L, 245R, 247A, 247D, 247E, 247F, 247M, 247N, 247Q, 247R, 247S, 247T, 247W, 247Y, 248F, 248P, 248Q, 248W, 249L, 249M, 249N, 249P, 249Y, 251H, 251I, 251W, 254D, 254E, 254F, 254G, 254H, 254I, 254K, 254L, 254M, 254N, 254P, 254Q, 254R, 254V, 254W, 254Y, 255K, 255N, 256H, 256I, 256K, 256L, 256V, 256W, 256Y, 257A, 257I, 257M, 257N, 257S, 258D, 260S, 262L, 264S, 265K, 265S, 267H, 267I, 267K, 268K, 269N, 269Q, 271T, 272H, 272K, 272L, 272R, 279A, 279D, 279F, 279G, 279H, 279I, 279K, 279L, 279M, 279N, 279Q, 279R, 279S, 279T, 279W, 279Y, 280T, 283F, 283G, 283H, 283I, 283K, 283L, 283M, 283P, 283R, 283T, 283W, 283Y, 285N, 286F, 288N, 288P, 292E, 292F, 292G, 292I, 292L, 293S, 293V, 301W, 304E, 307E, 307M, 312P, 315F, 315K, 315L, 315P, 315R, 316F, 316K, 317P, 317T, 318N, 318P, 318T, 332F, 332G, 332L, 332M, 332S, 332V, 332W, 339D, 339E, 339F, 339G, 339H, 339I, 339K, 339L, 339M, 339N, 339Q, 339R, 339S, 339W, 339Y, 341D, 341E, 341F, 341H, 341I, 341K, 341L, 341M, 341N, 341P, 341Q, 341R, 341S, 341T, 341V, 341W, 341Y, 343A, 343D, 343E, 343F, 343G, 343H, 343I, 343K, 343L, 343M, 343N, 343Q, 343R, 343S, 343T, 343V, 343W, 343Y, 373D, 373E, 373F, 373G, 373H, 373I, 373K, 373L, 373M, 373N, 373Q, 373R, 373S, 373T, 373V, 373W, 375R, 376E, 376F, 376G, 376H, 376I, 376L, 376M, 376N, 376P, 376Q, 376R, 376S, 376T, 376V, 376W, 376Y, 377G, 377K, 377P, 378N, 379N, 379Q, 379S, 379T, 380D, 380N, 380S, 380T, 382D, 382F, 382H, 382I, 382K, 382L, 382M, 382N, 382P, 382Q, 382R, 382S, 382T, 382V, 382W, 382Y, 385E, 385P, 386K, 423N, 424H, 424M, 424V, 426D, 426L, 427N, 429A, 429F, 429M, 430A, 430D, 430F, 430G, 430H, 430I, 430K, 430L, 430M, 430N, 430P, 430Q, 430R, 430S, 430T, 430V, 430W, 430Y, 431H, 431K, 431P, 432R, 432S, 438G, 438K, 438L, 438T, 438W, 439E, 439H, 439Q, 440D, 440E, 440F, 440G, 440H, 440I, 440K, 440L, 440M, 440Q, 440T, 440V or 442K. As above, the numbering of the residues in the heavy chain is that of the EU index (see Kabat et al., supra). Such variant Fc regions typically confer an altered effector function or altered serum half-life upon HRS polypeptide to which the variant Fc region is operably attached. Preferably the altered effector function is an increase in ADCC, a decrease in ADCC, an increase in CDC, a decrease in CDC, an increase in C1q binding affinity, a decrease in C1q binding affinity, an increase in FcR (preferably FcRn) binding affinity or a decrease in FcR (preferably FcRn) binding affinity as compared to a corresponding Fc region that lacks such amino acid substitution(s).

Additional examples include variant Fc regions that comprise an amino acid substitution at one or more of position(s) 221, 222, 224, 227, 228, 230, 231, 223, 233, 234, 235, 236, 237, 238, 239, 240, 241, 243, 244, 245, 246, 247, 249, 250, 258, 262, 263, 264, 265, 266, 267, 268, 269, 270, 271, 272, 273, 274, 275, 276, 278, 280, 281, 283, 285, 286, 288, 290, 291, 293, 294, 295, 296, 297, 298, 299, 300, 302, 313, 317, 318, 320, 322, 323, 324, 325, 326, 327, 328, 329, 330, 331, 332, 333, 334, 335 336 and/or 428 (see, e.g., U.S. Pat. No. 7,662,925). In specific embodiments, the variant Fc region comprises at least one amino acid substitution selected from the group consisting of: P230A, E233D, L234E, L234Y, L234I, L235D, L235S, L235Y, L235I, S239D, S239E, S239N, S239Q, S239T, V240I, V240M, F243L, V264I, V264T, V264Y, V266I, E272Y, K274T, K274E, K274R, K274L, K274Y, F275W, N276L, Y278T, V302I, E318R, S324D, S324I, S324V, N325T, K326I, K326T, L328M, L328I, L328Q, L328D, L328V, L328T, A330Y, A330L, A330I, I332D, I332E, I332N, I332Q, T335D, T335R, and T335Y. In other specific embodiments, the variant Fc region comprises at least one amino acid substitution selected from the group consisting of: V264I, F243L/V264I, L328M, I332E, L328M/I332E, V264I/I332E, S298A/I332E, S239E/I332E, S239Q/I332E, S239E, A330Y, I332D, L328I/I332E, L328Q/I332E, V264T, V240I, V266I, 5239D, S239D/I332D, S239D/I332E, S239D/I332N, S239D/I332Q, S239E/I332D, S239E/I332N, S239E/I332Q, S239N/I332D, S239N/I332E, S239Q/I332D, A330Y/I332E, V264I/A330Y/I332E, A330L/I332E, V264I/A330L/I332E, L234E, L234Y, L234I, L235D, L235S, L235Y, L235I, S239T, V240M, V264Y, A330I, N325T, L328D/I332E, L328V/I332E, L328T/I332E, L328I/I332E, S239E/V264I/I332E, S239Q/V264I/I332E, S239E/V264I/A330Y/I332E, S239D/A330Y/I332E, S239N/A330Y/I332E, S239D/A330L/I332E, S239N/A330L/I332E, V264I/S298A/I332E, S239D/S298A/I332E, S239N/S298A/I332E, S239D/V264I/I332E, S239D/V264I/S298A/I332E, S239D/V264I/A330L/I332E, S239D/I332E/A330I, P230A, P230A/E233D/I332E, E272Y, K274T, K274E, K274R, K274L, K274Y, F275W, N276L, Y278T, V302I, E318R, 5324D, S324I, S324V, K326I, K326T, T335D, T335R, T335Y, V240I/V266I, S239D/A330Y/I332E/L234I, S239D/A330Y/I332E/L235D, S239D/A330Y/I332E/V240I, S239D/A330Y/I332E/V264T, S239D/A330Y/I332E/K326E, and S239D/A330Y/I332E/K326T, In more specific embodiments, the variant Fc region comprises a series of substitutions selected from the group consisting of: N297D/I332E, F241Y/F243Y/V262T/V264T/N297D/I332E, S239D/N297D/I332E, S239E/N297D/I332E, S239D/D265Y/N297D/I332E, S239D/D265H/N297D/I332E, V264E/N297D/I332E, Y296N/N297D/I332E, N297D/A330Y/I332E, S239D/D265V/N297D/I332E, S239D/D265I/N297D/I332E, and N297D/S298A/A330Y/I332E. In specific embodiments, the variant Fc region comprises an amino acid substitution at position 332 (using the numbering of the EU index, Kabat et al., supra). Examples of substitutions include 332A, 332D, 332E, 332F, 332G, 332H, 332K, 332L, 332M, 332N, 332P, 332Q, 332R, 332S, 332T, 332V, 332W and 332Y. The numbering of the residues in the Fc region is that of the EU index of Kabat et al. Among other properties described herein, such variant Fc regions may have increased affinity for an FcγR, increased stability, and/or increased solubility, relative to a corresponding, wild-type Fc region.

Further examples include variant Fc regions that comprise one or more of the following amino acid substitutions: 224N/Y, 225A, 228L, 230S, 239P, 240A, 241L, 243S/L/G/H/I, 244L, 246E, 247L/A, 252T, 254T/P, 258K, 261Y, 265V, 266A, 267G/N, 268N, 269K/G, 273A, 276D, 278H, 279M, 280N, 283G, 285R, 288R, 289A, 290E, 291L, 292Q, 297D, 299A, 300H, 301C, 304G, 305A, 306I/F, 311R, 312N, 315D/K/S, 320R, 322E, 323A, 324T, 325S, 326E/R, 332T, 333D/G, 335I, 338R, 339T, 340Q, 341E, 342R, 344Q, 347R, 351S, 352A, 354A, 355W, 356G, 358T, 361D/Y, 362L, 364C, 365Q/P, 370R, 372L, 377V, 378T, 383N, 389S, 390D, 391C, 393A, 394A, 399G, 404S, 408G, 409R, 411I, 412A, 414M, 421S, 422I, 426F/P, 428T, 430K, 431S, 432P, 433P, 438L, 439E/R, 440G, 441F, 442T, 445R, 446A, 447E, optionally where the variant has altered recognition of an Fc ligand and/or altered effector function compared with a parent Fc polypeptide, and wherein the numbering of the residues is that of the EU index as in Kabat et al. Specific examples of these and related embodiments include variant Fc regions that comprise or consist of the following sets of substitutions: (1) N276D, R292Q, V305A, I377V, T394A, V412A and K439E; (2) P244L, K246E, D399G and K409R; (3) S304G, K320R, S324T, K326E and M358T; (4) F243S, P247L, D265V, V266A, S383N and T411I; (5) H224N, F243L, T393A and H433P; (6) V240A, S267G, G341E and E356G; (7) M252T, P291L, P352A, R355W, N390D, S408G, S426F and A431S; (8) P228L, T289A, L365Q, N389S and 5440G; (9) F241L, V273A, K340Q and L441F; (10) F241L, T299A, I332T and M428T; (11) E269K, Y300H, Q342R, V422I and G446A; (12) T225A, R301c, S304G, D312N, N315D, L351S and N421S; (13) S254T, L306I, K326R and Q362L; (14) H224Y, P230S, V323A, E333D, K338R and S364C; (15) T335I, K414M and P445R; (16) T335I and K414M; (17) P247A, E258K, D280N, K288R, N297D, T299A, K322E, Q342R, S354A and L365P; (18) H268N, V279M, A339T, N361D and S426P; (19) C261Y, K290E, L306F, Q311R, E333G and Q438L; (20) E283G, N315K, E333G, R344Q, L365P and S442T; (21) Q347R, N361Y and K439R; (22) S239P, S254P, S267N, H285R, N315S, F372L, A378T, N390D, Y391C, F404S, E430K, L432P and K447E; and (23) E269G, Y278H, N325S and K370R, wherein the numbering of the residues is that of the EU index as in Kabat et al. (see, e.g., U.S. Application No. 2010/0184959).

Variant Fc regions can also have one or more mutated hinge regions, as described, for example, in U.S. Application No. 2003/0118592. For instance, one or more cysteines in a hinge region can be deleted or substituted with a different amino acid. The mutated hinge region can comprise no cysteine residues, or it can comprise 1, 2, or 3 fewer cysteine residues than a corresponding, wild-type hinge region. In some embodiments, an Fc region having a mutated hinge region of this type exhibits a reduced ability to dimerize, relative to a wild-type Ig hinge region.

In particular embodiments, the Fc region comprises, consists, or consists essentially of the Fc from human IgG1 or IgG4 (see, e.g., Allberse and Schuurman, Immunology. 105:9-19, 2002), or a fragment or variant thereof. Table F1 below provides exemplary sequences (CH1, hinge (underlined), CH2, and CH3 regions) from human IgG1 and IgG4. Examples of variant IgG4 sequences that can be employed are described, for example, in Peters et al., JBC. 287:24525-24533, 2012, and include substitutions at C227, C230, C127 (e.g., C127S), and C131 (e.g., C131S). Other variants that can be used include a L445P substitution in IgG4 (denoted as IgG4-2) or a D356E and L358M substitution in IgG1, (denoted as IgG1m(zf)).

TABLE F1
Exemplary IgG4 Fc Sequences

| | | SEQ ID |
|---|---|---|
| Name | Sequence | NO: |
| Wild-type | ASTKGPSVFPLAPCSRSTSESTAALGCLVKDYFPEPVTVSWNSGALTSGV | 414 |
| IgG4 | HTFPAVLQSSGLYSLSSVVTVPSSSLGTKTYTCNVDHKPSNTKVDKRVES | |
| | KYGPPCPSCPAPEFLGGPSVFLFPPKPKDTLMISRTPEVTCVVVDVSQED | |
| | PEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGK | |
| S241P | ASTKGPSVFPLAPCSRSTSESTAALGCLVKDYFPEPVTVSWNSGALTSGV | 415 |
| | HTFPAVLQSSGLYSLSSVVTVPSSSLGTKTYTCNVDHKPSNTKVDKRVES | |
| | KYGPPCPPCPAPEFLGGPSVFLFPPKPKDTLMISRTPEVTCVVVDVSQED | |
| | PEVQFNWYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCLVK | |
| | GFYPSDIAVEWESNGQPENNYKTTPPVLDSDGSFFLYSRLTVDKSRWQEG | |
| | NVFSCSVMHEALHNHYTQKSLSLSLGK | |
| IgG1m(za) | ASTKGPSVFPLAPSSKSTSGGTAALGCLVKDYFPEPVTVSWNSGALTSGV | 416 |
| GenBank: | HTFPAVLQSSGLYSLSSVVTVPSSSLGTQTYICNVNHKPSNTKVDKKVEP | |
| AH007035.2 | KSCDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGK | |
| | EYKCKVSNKALPAPIEKTISKAKGQPREPQVYTLPPSRDELTKNQVSLTC | |
| | LVKGFYPSDIAVEWESNGQPENNYKTTPPVLDSDGSFFLYSKLTVDKSRW | |
| | QQGNVFSCSVMHEALHNHYTQKSLSLSPGK | |
| Kappa | RTVAAPSVFIFPPSDEQLKSGTASVVCLLNNFYPREAKVQWKVDNALQSG | 417 |
| Km3 | NSQESVTEQDSKDSTYSLSSTLTLSKADYEKHKVYACEVTHQGLSSPVTK | |
| | SFNRGEC | |

As noted above, antibodies having altered Fc regions typically have altered (e.g., improved, increased, decreased) pharmacokinetic properties relative to corresponding wild-type Fc region. Examples of pharmacokinetic properties include stability or half-life, bioavailability (the fraction of a drug that is absorbed), tissue distribution, volume of distribution (apparent volume in which a drug is distributed immediately after it has been injected intravenously and equilibrated between plasma and the surrounding tissues), concentration (initial or steady-state concentration of drug in plasma), elimination rate constant (rate at which drugs are removed from the body), elimination rate (rate of infusion required to balance elimination), area under the curve (AUC or exposure; integral of the concentration-time curve, after a single dose or in steady state), clearance (volume of plasma cleared of the drug per unit time), Cₘₐₓ (peak plasma concentration of a drug after oral administration), tₘₐₓ (time to reach Cₘₐₓ), Cₘₐₓ (lowest concentration that a drug reaches before the next dose is administered), and fluctuation (peak trough fluctuation within one dosing interval at steady state).

In particular embodiments, an antibody or antigen-binding fragment thereof has a biological half life at about pH 7.4, at about a physiological pH, at about 25° C. or room temperature, and/or at about 37° C. or human body temperature (e.g., in vivo, in serum, in a given tissue, in a given species such as rat, mouse, monkey, or human), of about or at least about 30 minutes, about 1 hour, about 2 hour, about 3 hours, about 4 hours, about 5 hours, about 6 hours, about 12 hours, about 18 hours, about 20 hours, about 24 hours, about 30 hours, about 36 hours, about 40 hours, about 48 hours, about 50 hours, about 60 hours, about 70 hours, about 72 hours, about 80 hours, about 84 hours, about 90 hours, about 96 hours, about 120 hours, or about 144 hours or more, or about 1 week, or about 2 weeks, or about 3 weeks, or about 4 weeks, or about 5 weeks, or about 6 weeks or more, or any intervening half-life, including all ranges in between.

In some embodiments, an antibody or antigen-binding fragment thereof has a Tₘ of about or at least about 60, 62, 64, 66, 68, 70, 72, 74, or 75° C. In some embodiments, an antibody or antigen-binding fragment thereof has a Tₘ of about 60° C. or greater.

In some embodiments, an antibody or antigen-binding fragment thereof conjugated to one or more cytotoxic or chemotherapeutic agents. General examples of cytotoxic or chemotherapeutic agents include, without limitation, alkylating agents, anti-metabolites, anthracyclines, anti-tumor antibiotics, platinums, type I topoisomerase inhibitors, type II topoisomerase inhibitors, vinca alkaloids, and taxanes.

Specific examples of cytotoxic or chemotherapeutic agents include, without limitation, cyclophosphamide, cilengitide, lomustine (CCNU), melphalan, procarbazine, carmustine (BCNU), enzastaurin, busulfan, daunorubicin, doxorubicin, gefitinib, erlotinib idarubicin, temozolomide, epirubicin, mitoxantrone, bleomycin, cisplatin, carboplatin, oxaliplatin, camptothecins, irinotecan, topotecan, amsacrine, etoposide, etoposide phosphate, teniposide, temsirolimus, everolimus, vincristine, vinblastine, vinorelbine, vindesine, CT52923, paclitaxel, imatinib, dasatinib, sorafenib, pazopanib, sunitnib, vatalanib, geftinib, erlotinib, AEE-788, dichoroacetate, tamoxifen, fasudil, SB-681323, semaxanib, donepizil, galantamine, memantine, rivastigmine, tacrine, rasigiline, naltrexone, lubiprostone, safinamide, istradefylline, pimavanserin, pitolisant, isradipine, pridopidine (ACR16), tetrabenazine, bexarotene, glatirimer acetate, fingolimod, and mitoxantrone, including pharmaceutically acceptable salts and acids thereof. Further examples of cytotoxic or chemotherapeutic agents include alkylating agents such as thiotepa, cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above.

The antibodies or antigen-binding fragments thereof can be used in any of the compositions, methods, and/or kits described herein, and combined with one or more of the immunotherapy agents described herein.

Immunotherapy Agents

Certain embodiments employ one or more cancer immunotherapy agents. In certain instances, an immunotherapy agent modulates the immune response of a subject, for example, to increase or maintain a cancer-related or cancer-specific immune response, and thereby results in increased immune cell inhibition or reduction of cancer cells. Exemplary immunotherapy agents include polypeptides, for example, antibodies and antigen-binding fragments thereof, ligands, and small peptides, and mixtures thereof. Also include as immunotherapy agents are small molecules, cells (e.g., immune cells such as T-cells), various cancer vaccines, gene therapy or other polynucleotide-based agents, including viral agents such as oncolytic viruses, and others known in the art. Thus, in certain embodiments, the cancer immunotherapy agent is selected from one or more of immune checkpoint modulatory agents, cancer vaccines, oncolytic viruses, cytokines, and a cell-based immunotherapies.

In certain embodiments, the cancer immunotherapy agent is an immune checkpoint modulatory agent. Particular examples include “antagonists” of one or more inhibitory immune checkpoint molecules, and “agonists” of one or more stimulatory immune checkpoint molecules. Generally, immune checkpoint molecules are components of the immune system that either turn up a signal (co-stimulatory molecules) or turn down a signal, the targeting of which has therapeutic potential in cancer because cancer cells can perturb the natural function of immune checkpoint molecules (see, e.g., Sharma and Allison, Science. 348:56-61, 2015; Topalian et al., Cancer Cell. 27:450-461, 2015; Pardoll, Nature Reviews Cancer. 12:252-264, 2012). In some embodiments, the immune checkpoint modulatory agent (e.g., antagonist, agonist) “binds” or “specifically binds” to the one or more immune checkpoint molecules, as described herein.

In particular embodiments, the immune checkpoint modulatory agent is a polypeptide or peptide. The terms “peptide” and “polypeptide” are used interchangeably herein, however, in certain instances, the term “peptide” can refer to shorter polypeptides, for example, polypeptides that consist of about 2, 3, 4, 5, 6, 7, 8, 9, 10, 11, 12, 13, 14, 15, 16, 17, 18, 19, 20, 25, 30, 35, 40, 45, or 50 amino acids, including all integers and ranges (e.g., 5-10, 8-12, 10-15) in between. Polypeptides and peptides can be composed of naturally-occurring amino acids and/or non-naturally occurring amino acids, as described herein

Antibodies are also included as polypeptides. Thus, in some embodiments, the immune checkpoint modulatory polypeptide agent is an antibody or “antigen-binding fragment thereof”, as described elsewhere herein.

In some embodiments, the agent is or comprises a “ligand,” for example, a natural ligand, of the immune checkpoint molecule. A “ligand” refers generally to a substance or molecule that forms a complex with a target molecule (e.g., biomolecule) to serve a biological purpose, and includes a “protein ligand,” which generally produces a signal by binding to a site on a target molecule or target protein. Thus, certain agents are protein ligands that, in nature, bind to an immune checkpoint molecule and produce a signal. Also included are “modified ligands,” for example, protein ligands that are fused to a pharmacokinetic modifier, for example, an Fc region derived from an immunoglobulin.

The binding properties of polypeptides can be quantified using methods well known in the art (see Davies et al., Annual Rev. Biochem. 59:439-473, 1990). In some embodiments, a polypeptide specifically binds to a target molecule, for example, an immune checkpoint molecule or an epitope thereof, with an equilibrium dissociation constant that is about or ranges from about ≤10−7 to about 10−8 M. In some embodiments, the equilibrium dissociation constant is about or ranges from about ≤10−9 M to about ≤10−10 M. In certain illustrative embodiments, the polypeptide has an affinity (Kd) for a target described herein (to which it specifically binds) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the agent is a “small molecule,” which refers to an organic compound that is of synthetic or biological origin (biomolecule), but is typically not a polymer. Organic compounds refer to a large class of chemical compounds whose molecules contain carbon, typically excluding those that contain only carbonates, simple oxides of carbon, or cyanides. A “biomolecule” refers generally to an organic molecule that is produced by a living organism, including large polymeric molecules (biopolymers) such as peptides, polysaccharides, and nucleic acids as well, and small molecules such as primary secondary metabolites, lipids, phospholipids, glycolipids, sterols, glycerolipids, vitamins, and hormones. A “polymer” refers generally to a large molecule or macromolecule composed of repeating structural units, which are typically connected by covalent chemical bond.

In certain embodiments, a small molecule has a molecular weight of about or less than about 1000-2000 Daltons, typically between about 300 and 700 Daltons, and including about or less than about 50, 100, 150, 200, 250, 300, 350, 400, 450, 500, 550, 500, 650, 600, 750, 700, 850, 800, 950, 1000 or 2000 Daltons.

Certain small molecules can have the “specific binding” characteristics described for herein polypeptides such as antibodies. For instance, in some embodiments a small molecule specifically binds to a target, for example, an immune checkpoint molecule, with a binding affinity (Kd) of about, at least about, or less than about, 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, or 50 nM.

In some embodiments, the immune checkpoint modulatory agent is an antagonist or inhibitor of one or more inhibitory immune checkpoint molecules. Exemplary inhibitory immune checkpoint molecules include Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, and T-cell immunoreceptor with Ig and ITIM domains (TIGIT).

In certain embodiments, the agent is a PD-1 (receptor) antagonist or inhibitor, the targeting of which has been shown to restore immune function in the tumor environment (see, e.g., Phillips et al., Int Immunol. 27:39-46, 2015). PD-1 is a cell surface receptor that belongs to the immunoglobulin superfamily and is expressed on T cells and pro-B cells. PD-1 interacts with two ligands, PD-L1 and PD-L2. PD-1 functions as an inhibitory immune checkpoint molecule, for example, by reducing or preventing the activation of T-cells, which in turn reduces autoimmunity and promotes self-tolerance. The inhibitory effect of PD-1 is accomplished at least in part through a dual mechanism of promoting apoptosis in antigen specific T-cells in lymph nodes while also reducing apoptosis in regulatory T cells (suppressor T cells). Some examples of PD-1 antagonists or inhibitors include an antibody or antigen-binding fragment or small molecule that specifically binds to PD-1 and reduces one or more of its immune-suppressive activities, for example, its downstream signaling or its interaction with PD-L1. Specific examples of PD-1 antagonists or inhibitors include the antibodies nivolumab, pembrolizumab, PDR001, MK-3475, AMP-224, AMP-514, and pidilizumab, and antigen-binding fragments thereof (see, e.g., U.S. Pat. Nos. 8,008,449; 8,993,731; 9,073,994; 9,084,776; 9,102,727; 9,102,728; 9,181,342; 9,217,034; 9,387,247; 9,492,539; 9,492,540; and U.S. Application Nos. 2012/0039906; 2015/0203579).

In some embodiments, the agent is a PD-L1 antagonist or inhibitor. As noted above, PD-L1 is one of the natural ligands for the PD-1 receptor. General examples of PD-L1 antagonists or inhibitors include an antibody or antigen-binding fragment or small molecule that specifically binds to PD-L1 and reduces one or more of its immune-suppressive activities, for example, its binding to the PD-1 receptor. Specific examples of PD-L1 antagonists include the antibodies atezolizumab (MPDL3280A), avelumab (MSB0010718C), and durvalumab (MEDI4736), and antigen-binding fragments thereof (see, e.g., U.S. Pat. Nos. 9,102,725; 9,393,301; 9,402,899; 9,439,962).

In some embodiments, the agent is a PD-L2 antagonist or inhibitor. As noted above, PD-L2 is one of the natural ligands for the PD-1 receptor. General examples of PD-L2 antagonists or inhibitors include an antibody or antigen-binding fragment or small molecule that specifically binds to PD-L2 and reduces one or more of its immune-suppressive activities, for example, its binding to the PD-1 receptor.

In some embodiments, the agent is a CTLA-4 antagonist or inhibitor. CTLA4 or CTLA-4 (cytotoxic T-lymphocyte-associated protein 4), also known as CD152 (cluster of differentiation 152), is a protein receptor that functions as an inhibitory immune checkpoint molecule, for example, by transmitting inhibitory signals to T-cells when it is bound to CD80 or CD86 on the surface of antigen-presenting cells. General examples CTLA-4 antagonists or inhibitors include an antibody or antigen-binding fragment or small molecule that specifically binds to CTLA-4. Particular examples include the antibodies ipilimumab and tremelimumab, and antigen-binding fragments thereof. At least some of the activity of ipilimumab is believed to be mediated by antibody-dependent cell-mediated cytotoxicity (ADCC) killing of suppressor Tregs that express CTLA-4.

In some embodiments, the agent is an IDO antagonist or inhibitor, or a TDO antagonist or inhibitor. IDO and TDO are tryptophan catabolic enzymes with immune-inhibitory properties. For example, IDO is known to suppress T-cells and NK cells, generate and activate Tregs and myeloid-derived suppressor cells, and promote tumor angiogenesis. General examples of IDO and TDO antagonists or inhibitors include an antibody or antigen-binding fragment or small molecule that specifically binds to IDO or TDO (see, e.g., Platten et al., Front Immunol. 5: 673, 2014) and reduces or inhibits one or more immune-suppressive activities. Specific examples of IDO antagonists or inhibitors include indoximod (NLG-8189), 1-methyl-tryptophan (1MT), β-Carboline (norharmane; 9H-pyrido[3,4-b]indole), rosmarinic acid, and epacadostat (see, e.g., Sheridan, Nature Biotechnology. 33:321-322, 2015). Specific examples of TDO antagonists or inhibitors include 680C91 and LM10 (see, e.g., Pilotte et al., PNAS USA. 109:2497-2502, 2012).

In some embodiments, the agent is a TIM-3 antagonist or inhibitor. T-cell Immunoglobulin domain and Mucin domain 3 (TIM-3) is expressed on activated human CD4+ T-cells and regulates Th1 and Th17 cytokines. TIM-3 also acts as a negative regulator of Th1/Tc1 function by triggering cell death upon interaction with its ligand, galectin-9. TIM-3 contributes to the suppressive tumor microenvironment and its overexpression is associated with poor prognosis in a variety of cancers (see, e.g., Li et al., Acta Oncol. 54:1706-13, 2015). General examples of TIM-3 antagonists or inhibitors include an antibody or antigen-binding fragment or small molecule that specifically binds to TIM-3 and reduces or inhibits one or more of its immune-suppressive activities.

In some embodiments, the agent is a LAG-3 antagonist or inhibitor. Lymphocyte Activation Gene-3 (LAG-3) is expressed on activated T-cells, natural killer cells, B-cells and plasmacytoid dendritic cells. It negatively regulates cellular proliferation, activation, and homeostasis of T-cells, in a similar fashion to CTLA-4 and PD-1 (see, e.g., Workman and Vignali. European Journal of Immun. 33: 970-9, 2003; and Workman et al., Journal of Immun. 172: 5450-5, 2004), and has been reported to play a role in Treg suppressive function (see, e.g., Huang et al., Immunity. 21: 503-13, 2004). LAG3 also maintains CD8+ T-cells in a tolerogenic state and combines with PD-1 to maintain CD8 T-cell exhaustion. General examples of LAG-3 antagonists or inhibitors include an antibody or antigen-binding fragment or small molecule that specifically binds to LAG-3 and inhibits one or more of its immune-suppressive activities. Specific examples include the antibody BMS-986016, and antigen-binding fragments thereof.

In some embodiments, the agent is a VISTA antagonist or inhibitor. V-domain Ig suppressor of T cell activation (VISTA) is primarily expressed on hematopoietic cells and is an inhibitory immune checkpoint regulator that suppresses T-cell activation, induces Foxp3 expression, and is highly expressed within the tumor microenvironment where it suppresses anti-tumor T cell responses (see, e.g., Lines et al., Cancer Res. 74:1924-32, 2014). General examples of VISTA antagonists or inhibitors include an antibody or antigen-binding fragment or small molecule that specifically binds to VISTA and reduces one or more of its immune-suppressive activities.

In some embodiments, the agent is a BTLA antagonist or inhibitor. B- and T-lymphocyte attenuator (BTLA; CD272) expression is induced during activation of T-cells, and it inhibits T-cells via interaction with tumor necrosis family receptors (TNF-R) and B7 family of cell surface receptors. BTLA is a ligand for tumor necrosis factor (receptor) superfamily, member 14 (TNFRSF14), also known as herpes virus entry mediator (HVEM). BTLA-HVEM complexes negatively regulate T-cell immune responses, for example, by inhibiting the function of human CD8+ cancer-specific T-cells (see, e.g., Derre et al., J Clin Invest 120:157-67, 2009). General examples of BTLA antagonists or inhibitors include an antibody or antigen-binding fragment or small molecule that specifically binds to BTLA-4 and reduce one or more of its immune-suppressive activities.

In some embodiments, the agent is an HVEM antagonist or inhibitor, for example, an antagonist or inhibitor that specifically binds to HVEM and interferes with its interaction with BTLA or CD160. General examples of HVEM antagonists or inhibitors include an antibody or antigen-binding fragment or small molecule that specifically binds to HVEM, optionally reduces the HVEM/BTLA and/or HVEM/CD160 interaction, and thereby reduces one or more of the immune-suppressive activities of HVEM.

In some embodiments, the agent is a CD160 antagonist or inhibitor, for example, an antagonist or inhibitor that specifically binds to CD160 and interferes with its interaction with HVEM. General examples of CD160 antagonists or inhibitors include an antibody or antigen-binding fragment or small molecule that specifically binds to CD160, optionally reduces the CD160/HVEM interaction, and thereby reduces or inhibits one or more of its immune-suppressive activities.

In some embodiments, the agent is a TIGIT antagonist or inhibitor. T cell Ig and ITIM domain (TIGIT) is a coinhibitory receptor that is found on the surface of a variety of lymphoid cells, and suppresses antitumor immunity, for example, via Tregs (Kurtulus et al., J Clin Invest. 125:4053-4062, 2015). General examples of TIGIT antagonists or inhibitors include an antibody or antigen-binding fragment or small molecule that specifically binds to TIGIT and reduce one or more of its immune-suppressive activities (see, e.g., Johnston et al., Cancer Cell. 26:923-37, 2014).

In certain embodiments, the immune checkpoint modulatory agent is an agonist of one or more stimulatory immune checkpoint molecules. Exemplary stimulatory immune checkpoint molecules include OX40, CD40, Glucocorticoid-Induced TNFR Family Related Gene (GITR), CD137 (4-1BB), CD27, CD28, CD226, and Herpes Virus Entry Mediator (HVEM).

In some embodiments, the agent is an OX40 agonist. OX40 (CD134) promotes the expansion of effector and memory T cells, and suppresses the differentiation and activity of T-regulatory cells (see, e.g., Croft et al., Immunol Rev. 229:173-91, 2009). Its ligand is OX40L (CD252). Since OX40 signaling influences both T-cell activation and survival, it plays a key role in the initiation of an anti-tumor immune response in the lymph node and in the maintenance of the anti-tumor immune response in the tumor microenvironment. General examples of OX40 agonists include an antibody or antigen-binding fragment or small molecule or ligand that specifically binds to OX40 and increases one or more of its immunostimulatory activities. Specific examples include OX86, OX-40L, Fc-OX40L, GSK3174998, MEDI0562 (a humanized OX40 agonist), MEDI6469 (murine OX4 agonist), and MEDI6383 (an OX40 agonist), and antigen-binding fragments thereof.

In some embodiments, the agent is a CD40 agonist. CD40 is expressed on antigen-presenting cells (APC) and some malignancies. Its ligand is CD40L (CD154). On APC, ligation results in upregulation of costimulatory molecules, potentially bypassing the need for T-cell assistance in an antitumor immune response. CD40 agonist therapy plays an important role in APC maturation and their migration from the tumor to the lymph nodes, resulting in elevated antigen presentation and T cell activation. Anti-CD40 agonist antibodies produce substantial responses and durable anticancer immunity in animal models, an effect mediated at least in part by cytotoxic T-cells (see, e.g., Johnson et al. Clin Cancer Res. 21: 1321-1328, 2015; and Vonderheide and Glennie, Clin Cancer Res. 19:1035-43, 2013). General examples of CD40 agonists include an antibody or antigen-binding fragment or small molecule or ligand that specifically binds to CD40 and increases one or more of its immunostimulatory activities. Specific examples include CP-870,893, dacetuzumab, Chi Lob 7/4, ADC-1013, CD40L, rhCD40L, and antigen-binding fragments thereof.

In some embodiments, the agent is a GITR agonist. Glucocorticoid-Induced TNFR family Related gene (GITR) increases T cell expansion, inhibits the suppressive activity of Tregs, and extends the survival of T-effector cells. GITR agonists have been shown to promote an anti-tumor response through loss of Treg lineage stability (see, e.g., Schaer et al., Cancer Immunol Res. 1:320-31, 2013). These diverse mechanisms show that GITR plays an important role in initiating the immune response in the lymph nodes and in maintaining the immune response in the tumor tissue. Its ligand is GITRL. General examples of GITR agonists include an antibody or antigen-binding fragment or small molecule or ligand that specifically binds to GITR and increases one or more of its immunostimulatory activities. Specific examples include GITRL, INCAGN01876, DTA-1, MEDI1873, and antigen-binding fragments thereof.

In some embodiments, the agent is a CD137 agonist. CD137 (4-1BB) is a member of the tumor necrosis factor (TNF) receptor family, and crosslinking of CD137 enhances T-cell proliferation, IL-2 secretion, survival, and cytolytic activity. CD137-mediated signaling also protects T-cells such as CD8+ T-cells from activation-induced cell death. General examples of CD137 agonists include an antibody or antigen-binding fragment or small molecule or ligand that specifically binds to CD137 and increases one or more of its immunostimulatory activities. Specific examples include the CD137 (or 4-1BB) ligand (see, e.g., Shao and Schwarz, J Leukoc Biol. 89:21-9, 2011) and the antibody utomilumab, including antigen-binding fragments thereof.

In some embodiments, the agent is a CD27 agonist. Stimulation of CD27 increases antigen-specific expansion of naïve T cells and contributes to T-cell memory and long-term maintenance of T-cell immunity. Its ligand is CD70. The targeting of human CD27 with an agonist antibody stimulates T-cell activation and antitumor immunity (see, e.g., Thomas et al., Oncoimmunology. 2014; 3:e27255. doi:10.4161/onci.27255; and He et al., J Immunol. 191:4174-83, 2013). General examples of CD27 agonists include an antibody or antigen-binding fragment or small molecule or ligand that specifically binds to CD27 and increases one or more of its immunostimulatory activities. Specific examples include CD70 and the antibodies varlilumab and CDX-1127 (1F5), including antigen-binding fragments thereof.

In some embodiments, the agent is a CD28 agonist. CD28 is constitutively expressed CD4+ T cells some CD8+ T cells. Its ligands include CD80 and CD86, and its stimulation increases T-cell expansion. General examples of CD28 agonists include an antibody or antigen-binding fragment or small molecule or ligand that specifically binds to CD28 and increases one or more of its immunostimulatory activities. Specific examples include CD80, CD86, the antibody TAB08, and antigen-binding fragments thereof.

In some embodiments, the agent is CD226 agonist. CD226 is a stimulating receptor that shares ligands with TIGIT, and opposite to TIGIT, engagement of CD226 enhances T-cell activation (see, e.g., Kurtulus et al., J Clin Invest. 125:4053-4062, 2015; Bottino et al., J Exp Med. 1984:557-567, 2003; and Tahara-Hanaoka et al., Int Immunol. 16:533-538, 2004). General examples of CD226 agonists include an antibody or antigen-binding fragment or small molecule or ligand (e.g., CD112, CD155) that specifically binds to CD226 and increases one or more of its immunostimulatory activities.

In some embodiments, the agent is an HVEM agonist. Herpesvirus entry mediator (HVEM), also known as tumor necrosis factor receptor superfamily member 14 (TNFRSF14), is a human cell surface receptor of the TNF-receptor superfamily. HVEM is found on a variety of cells including T-cells, APCs, and other immune cells. Unlike other receptors, HVEM is expressed at high levels on resting T-cells and down-regulated upon activation. It has been shown that HVEM signaling plays a crucial role in the early phases of T-cell activation and during the expansion of tumor-specific lymphocyte populations in the lymph nodes. General examples of HVEM agonists include an antibody or antigen-binding fragment or small molecule or ligand that specifically binds to HVEM and increases one or more of its immunostimulatory activities.

In certain embodiments, the cancer immunotherapy agent is a cancer vaccine. Exemplary cancer vaccines include Oncophage, human papillomavirus HPV vaccines such Gardasil or Cervarix, hepatitis B vaccines such as Engerix-B, Recombivax HB, or Twinrix, and sipuleucel-T (Provenge). In some embodiments, the cancer vaccine comprises or utilizes one or more cancer antigens, or cancer-associate d antigens. Exemplary cancer antigens include, without limitation, human Her2/neu, Her1/EGF receptor (EGFR), Her3, A33 antigen, B7H3, CD5, CD19, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PSMA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

In certain embodiments, the cancer immunotherapy agent is an oncolytic virus. An oncolytic virus is a virus that preferentially infects and kills cancer cells. Included are naturally-occurring and man-made or engineered oncolytic viruses. Most oncolytic viruses are engineered for tumor selectivity, although there are naturally-occurring examples such as Reovirus and the SVV-001 Seneca Valley virus. General examples of oncolytic viruses include VSV, Poliovirus, Reovirus, Senecavirus, and RIGVIR, and engineered versions thereof. Non-limiting examples of oncolytic viruses include herpes simplex virus (HSV) and engineered version thereof, talimogene laherparepvec (T-VEC), coxsackievirus A21 (CAVATAK™), Oncorine (H101), pelareorep (REOLYSIN®), Seneca Valley virus (NTX-010), Senecavirus SVV-001, ColoAd1, SEPREHVIR (HSV-1716), CGTG-102 (Ad5/3-D24-GMCSF), GL-ONC1, MV-NIS, and DNX-2401, among others.

In certain embodiments, the cancer immunotherapy agent is a cytokine. Exemplary cytokines include interferon (IFN)-α, IL-2, IL-12, IL-7, IL-21, and Granulocyte-macrophage colony-stimulating factor (GM-CSF).

In certain embodiments, the cancer immunotherapy agent is cell-based immunotherapy, for example, a T-cell based adoptive immunotherapy. In some embodiments, the cell-based immunotherapy comprises cancer antigen-specific T-cells, optionally ex vivo-derived T-cells. In some embodiments, the cancer antigen-specific T-cells are selected from one or more of chimeric antigen receptor (CAR)-modified T-cells, and T-cell Receptor (TCR)-modified T-cells, tumor infiltrating lymphocytes (TILs), and peptide-induced T-cells. In specific embodiments, the CAR-modified T-cell is targeted against CD-19 (see, e.g., Maude et al., Blood. 125:4017-4023, 2015).

In certain instances, the cancer to be treated associates with the cancer antigen, that is, the cancer antigen-specific T-cells are targeted against or enriched for at least one antigen that is known to associate with the cancer to be treated. In some embodiments, the cancer antigen is selected from one or more of CD19, human Her2/neu, Her1/EGF receptor (EGFR), Her3, A33 antigen, B7H3, CD5, CD20, CD22, CD23 (IgE Receptor), MAGE-3, C242 antigen, 5T4, IL-6, IL-13, vascular endothelial growth factor VEGF (e.g., VEGF-A) VEGFR-1, VEGFR-2, CD30, CD33, CD37, CD40, CD44, CD51, CD52, CD56, CD74, CD80, CD152, CD200, CD221, CCR4, HLA-DR, CTLA-4, NPC-1C, tenascin, vimentin, insulin-like growth factor 1 receptor (IGF-1R), alpha-fetoprotein, insulin-like growth factor 1 (IGF-1), carbonic anhydrase 9 (CA-IX), carcinoembryonic antigen (CEA), guanylyl cyclase C, NY-ESO-1, p53, survivin, integrin αvβ3, integrin α5β1, folate receptor 1, transmembrane glycoprotein NMB, fibroblast activation protein alpha (FAP), glycoprotein 75, TAG-72, MUC1, MUC16 (or CA-125), phosphatidylserine, prostate-specific membrane antigen (PMSA), NR-LU-13 antigen, TRAIL-R1, tumor necrosis factor receptor superfamily member 10b (TNFRSF10B or TRAIL-R2), SLAM family member 7 (SLAMF7), EGP40 pancarcinoma antigen, B-cell activating factor (BAFF), platelet-derived growth factor receptor, glycoprotein EpCAM (17-1A), Programmed Death-1, protein disulfide isomerase (PDI), Phosphatase of Regenerating Liver 3 (PRL-3), prostatic acid phosphatase, Lewis-Y antigen, GD2 (a disialoganglioside expressed on tumors of neuroectodermal origin), glypican-3 (GPC3), and mesothelin.

Additional exemplary cancer antigens include 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA15-3/CA 27-29, CA19-9, CA72-4, CA125, calreticulin, CAMEL, CASP-8/m, cathepsin B, cathepsin L, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp 100, GPC3, GPNMB/m, HAGE, HAST-2, hepsin, Her2/neu, HERV-K-MEL, HLA-A*0201-R1 7I, HLA-A1 1/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1 R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MCI R, M-CSF, ME1/m, mesothelin, MG50/PXDN, MMP1 1, MN/CA IX-antigen, MRP-3, MUC-1, MUC-2, MUM-1/m, MUM-2/m, MUM-3/m, myosin class 1/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, NY-ESO-1, OA1, OFA-iLRP, OGT, OGT/m, OS-9, OS-9/m, osteocalcin, osteopontin, pi 5, p190 minor bcr-abl, p53, p53/m, PAGE-4, PAI-1, PAI-2, PAP, PART-1, PATE, PDEF, Pim-1 Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD1 68, RU1, RU2, S-100, SAGE, SART-1, SART-2, SART-3, SCC, SIRT2/m, Sp1 7, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGF-beta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1. Certain preferred antigens include p53, CA125, EGFR, Her2/neu, hTERT, PAP, MAGE-A1, MAGE-A3, Mesothelin, MUC-1, GP100, MART-1, Tyrosinase, PSA, PSCA, PSMA, STEAP-1, Ras, CEA and WT1, and more preferably PAP, MAGE-A3, WT1, and MUC-1.

In some embodiments the antigen is selected from MAGE-A1 (e.g., MAGE-A1 according to accession number M77481), MAGE-A2, MAGE-A3, MAGE-A6 (e.g., MAGE-A6 according to accession number NM_005363), MAGE-C1, MAGE-C2, melan-A (e.g., melan-A according to accession number NM_00551 1), GP100 (e.g., GP100 according to accession number M77348), tyrosinase (e.g. tyrosinase according to accession number NM_000372), survivin (e.g. survivin according to accession number AF077350), CEA (e.g., CEA according to accession number NM_004363), Her-2/neu (e.g., Her-2/neu according to accession number M1 1 730), WT1 (e.g., WT1 according to accession number NM_000378), PRAME (e.g., PRAME according to accession number NM_0061 15), EGFRI (epidermal growth factor receptor 1) (e.g., EGFRI (epidermal growth factor receptor 1) according to accession number AF288738), MUC1, mucin-1 (e.g. mucin-1 according to accession number NM_002456), SEC61 G (e.g., SEC61 G according to accession number NM_014302), hTERT (e.g., hTERT accession number NM_198253), 5T4 (e.g. 5T4 according to accession number NM_006670), TRP-2 (e.g., TRP-2 according to accession number NM_001 922), STEAP 1 (Six-transmembrane epithelial antigen of prostate 1), PSCA, PSA, PSMA, etc.

In some embodiments, the cancer antigen is selected from PCA, PSA, PSMA, STEAP, and optionally MUC-1, including fragments, variants, and derivatives thereof. In some embodiments, the cancer antigen selected from NY-ESO-1, MAGE-C1, MAGE-C2, survivin, 5T4, and optionally MUC-1, including fragments, variants, and derivatives thereof.

In some instances, cancer antigens encompass idiotypic antigens associated with a cancer or tumor disease, particularly lymphoma or a lymphoma associated disease, for example, wherein the idiotypic antigen is an immunoglobulin idiotype of a lymphoid blood cell or a T cell receptor idiotype of a lymphoid blood cell.

In some instances, the cancer antigen-specific T-cells are selected from one or more of chimeric antigen receptor (CAR)-modified T-cells (e.g., targeted against a cancer antigen), and T-cell Receptor (TCR)-modified T-cells, tumor infiltrating lymphocytes (TILs), and peptide-induced T-cells.

The skilled artisan will appreciate that the various cancer immunotherapy agents described herein can be combined with any one or more of the various anti-HRS antibodies (including antigen-binding fragments thereof) described herein, and used according to any one or more of the methods or compositions described herein.

Methods of Use and Therapeutic Compositions

As noted above, embodiments of the present disclosure relate to the discovery that antibodies against human histidyl-tRNA synthetase (HRS) have unexpected immunomodulatory properties that are relevant to treating cancers. Accordingly, antibodies directed against human HRS can be used as standalone therapies in the treatment of cancer, or in combination with cancer immunotherapies.

Certain embodiments therefore include methods of treating ameliorating the symptoms of, or inhibiting the progression of, a cancer in a subject in need thereof, comprising administering to the subject at least one antibody or antigen-binding fragment thereof that specifically binds to a human HRS polypeptide (an anti-HRS antibody). Certain embodiments include reducing or preventing the re-emergence of a cancer in a subject in need thereof, for example, a metastatic cancer, wherein administration of the therapeutic composition enables generation of an immune memory to the cancer. In some embodiments, the subject has or is at risk for developing diabetes, for example, type 1 diabetes or type 2 diabetes. Also included are methods of treating cancer in a non-human mammalian subject, comprising administering a veterinary therapeutic composition comprising at least one antibody or antigen-binding fragment thereof specifically binds to a non-human mammalian HRS polypeptide, for example, selected from Table H2, including a dog, cat, pig, horse, or monkey HRS polypeptide.

Exemplary anti-HRS antibodies, including naturally-occurring antibodies and polyclonal mixtures thereof, and therapeutic compositions and Intravenous Immunoglobulin (IVIG) preparations comprising the same, are described elsewhere herein.

In certain aspects, it is hypothesized in a non-limiting way that anti-HRS antibodies, by blocking and/or clearing free full-length HRS in systemic circulation, in a tissue, at the cell surface, or within an endosome, may remove a previously-unrecognized inhibitory, immunomodulatory action of one or more systemic HRS polypeptides. This inhibitory activity of systemic HRS may function to restrict local autoimmune responses and immune activation associated with immune responses, and reduce robust immune responses to certain cancers.

Accordingly, certain embodiments relate to methods and compositions for reducing the levels of systemic one or more HRS polypeptides in circulation (selected, for example, from Table H1). In some embodiments, the subject has, and/or is selected for treatment based on having, circulating or serum levels of at least one HRS polypeptide, either bound or free, relative to the levels of a healthy or matched control population of subject(s). In some embodiments, the levels are about or at least about 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 3000, 4000, or 5000 pM of the at least one HRS polypeptide, or about or at least about 30-100, 40-100, 50-100, 30-2000, 40-2000, 50-2000, 60-2000, 70-2000, 80-2000, 90-2000, 100-2000, 200-2000, 300-2000, 400-2000, 500-2000, 600-2000, 700-2000, 800-2000, 900-2000, 1000-2000, 2000-3000, 3000-4000, or 4000-5000 pM of the at least one HRS polypeptide. In some embodiments, the subject has, and/or is selected for treatment based on having, a cancer which has increased levels or expression of an HRS polypeptide (selected, for example, from Table H1) and/or a coding mRNA thereof relative to a non-cancerous control cell or tissue. For instance, in some embodiments, the levels of an HRS polypeptide in the cancer cells or tissue are about or at least about 1.5, 2, 3, 4, 5, 6, 7, 8, 9, 10, 15, 20, 30, 40, 50, 60, 70, 80, 90, 100, 1000 or more times the levels of HRS polypeptide in a non-cancerous control or standard. Thus, certain embodiments include methods of selecting a subject for cancer treatment, comprising (i) detecting increased expression levels of an HRS polypeptide and/or coding mRNA in the subject relative to a control or reference, and (ii) administering to the subject a therapeutic composition comprising at least one HRS-antibody, as described herein. In particular embodiments, the HRS polypeptide is selected from one or more of SV9, SV11, and SV14.

In some embodiments, the subject has, and/or is selected for treatment based on having, increased circulating or serum levels of a soluble neuropilin 2 (NP2) polypeptide (selected, for example, from Table N1), either bound or free, relative to the levels of a healthy or matched control population of subject(s). For instance, in certain embodiments, the circulating or serum levels are about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NP2 polypeptide, or the circulating or serum levels are about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NP2 polypeptide. In some embodiments, the subject has, and/or is selected for treatment based on having, a cancer which has increased levels or expression of a soluble NP2 polypeptide (selected, for example, from Table N1) and/or a coding mRNA thereof relative to a non-cancerous control cell or tissue, optionally relative to a non-cancerous cell or tissue of the same type as the cancer. For instance, in some embodiments, the levels of the soluble NP2 polypeptide in the cancer cells or tissue are about or at least about 1.5, 2, 3, 4, 5, 6, 7, 8, 9, 10, 15, 20, 30, 40, 50, 60, 70, 80, 90, 100 or more times the levels of NP2 polypeptide in a non-cancerous control or standard. Some embodiments thus include methods of selecting a subject for cancer treatment, comprising (i) detecting increased expression levels of a soluble NP2 polypeptide and/or a coding mRNA thereof in the subject relative to a control or reference, and (ii) administering to the subject a therapeutic composition comprising at least one HRS-antibody, as described herein.

In some embodiments, the subject has, and/or is selected for treatment based on having, increased circulating levels of HRS:NP2 complexes relative to a healthy or matched control standard or population of subject(s). Certain embodiments therefore include methods of selecting a subject for cancer treatment, comprising (i) detecting increased expression levels of HRS:NP2 complexes in the subject relative to a control or reference, and (ii) administering to the subject a therapeutic composition comprising at least one HRS-antibody, as described herein.

In certain embodiments, administration of the at least one anti-HRS antibody increases the rate of clearance of an HRS polypeptide, or decreases the circulating levels of an HRS polypeptide, in the serum of a subject relative to pre-dosing levels of the HRS polypeptide, for example, by about or at least about 100, 200, 300, 400, or 500%. In certain embodiments, administration of the at least one anti-HRS antibody decreases the rate of clearance of an HRS polypeptide, or increases the circulating levels of an HRS polypeptide, in the serum of a subject relative to pre-dosing levels of the HRS polypeptide, for example, by about or at least about 100, 200, 300, 400, or 500%.

Particular embodiments method of reducing the average or maximum levels of at least one serum or circulating HRS polypeptide (selected, for example, from Table H1) to about or less than about 500 pM, 400 pM, 300 pM, 200 pM, 100 pM, 50 pm, 40 pM, 30 pM, 20 pM, or 10 pM, comprising administering an anti-HRS antibody to the subject in an amount and at a frequency sufficient to achieve the reduction. Some embodiments comprise administering an anti-HRS antibody to a subject in an amount and at a frequency sufficient to achieve an average, sustained blood plasma concentration of free, circulating HRS of about or less than about 500 pM, 400 pM, 300 pM, 200 pM, 100 pM, 50 pm, 40 pM, 30 pM, 20 pM, or 10 pM. In some embodiments, an anti-HRS antibody is administered to a subject in an amount and at a frequency sufficient to maintain an average, sustained blood plasma concentration of free, circulating full-length HRS of between about 10 pM and about 1 nM, or between about 10 pM and about 500 pM, or between about 10 pM and about 400 pM, or between about 10 pM and about 300 pM, or between about 10 pM and about 200 pM, or between about 10 pM and about 100 pM, or between about 10 pM and about 50 pM. In some embodiments, an anti-HRS antibody is administered to a subject in an amount and at a frequency sufficient to maintain an average, sustained blood plasma concentration of free, circulating full-length HRS of about or less than about 50 pM, or about or less than about 10 pM.

Some embodiments comprise administering at least one anti-HRS antibody to a subject in an amount and at a frequency sufficient to achieve an average, sustained blood plasma concentration of soluble NP2 of about or less than about 500 pM, 400 pM, 300 pM, 200 pM, 100 pM, 50 pm, 40 pM, 30 pM, 20 pM, or 10 pM.

Certain embodiments comprise administering at least one anti-HRS antibody in an amount and at a frequency sufficient to achieve a reduction in the circulating levels of HRS:NP2 complexes, for example, a reduction of about or at least about 5, 10, 15, 20, 25, 30, 35, 40, 45, 50, 60, 70, 80, 90, 95, 99, or 100%.

In some instances, an anti-HRS antibody enhances the immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control. In certain embodiments, an anti-HRS antibody enhances an adaptive immune response to the cancer, and in some embodiments, an anti-HRS antibody enhances an innate immune response to the cancer. In some-instances, an anti-HRS antibody enhances a T-cell-mediated response to the cancer. In some-instances, an anti-HRS antibody enhances a B-cell-mediated or antibody-mediated response to the cancer.

Some embodiments include administering the at least one anti-HRS antibody in an amount and at a frequency sufficient to achieve a steady state concentration, or average circulating concentration, of the at least one anti-HRS antibody of between about 1 nM and about 1 μM, between about 1 nM and about 100 nM, between about 1 nM and about 10 nM, or between about 1 nM and about 3 M.

Also include are combination therapies for treating cancers, including methods of treating ameliorating the symptoms of, or inhibiting the progression of, a cancer in a subject in need thereof, comprising administering to the subject at least one antibody or antigen-binding fragment thereof that specifically binds to a human HRS polypeptide (an anti-HRS antibody) in combination with at least one cancer immunotherapy agent. Exemplary cancer immunotherapy agents are described elsewhere herein.

In some instances, an anti-HRS antibody and the cancer immunotherapy agent are administered separately, for example, in separate therapeutic compositions and at the same or different times. In some embodiments, an anti-HRS antibody and the cancer immunotherapy agent are administered as part of the same therapeutic composition, at the same time.

In some embodiments, the methods and therapeutic compositions described herein (for example, anti-HRS antibody, alone or in combination with cancer immunotherapy agent) increase median survival time of a subject by 4 weeks, 5 weeks, 6 weeks, 7 weeks, 8 weeks, 9 weeks, 10 weeks, 15 weeks, 20 weeks, 25 weeks, 30 weeks, 40 weeks, or longer. In certain embodiments, the methods and therapeutic compositions described herein (for example, anti-HRS antibody, alone or in combination with cancer immunotherapy agent) increase median survival time of a subject by 1 year, 2 years, 3 years, or longer. In some embodiments, the methods and therapeutic compositions described herein (for example, anti-HRS antibody, alone or in combination with cancer immunotherapy agent) increase progression-free survival by 2 weeks, 3 weeks, 4 weeks, 5 weeks, 6 weeks, 7 weeks, 8 weeks, 9 weeks, 10 weeks or longer. In certain embodiments, the methods or therapeutic compositions described herein increase progression-free survival by 1 year, 2 years, 3 years, or longer.

In certain embodiments, the methods and therapeutic compositions described herein (for example, anti-HRS antibody, alone or in combination with cancer immunotherapy agent) are sufficient to result in tumor regression, as indicated by a statistically significant decrease in the amount of viable tumor, for example, at least a 10%, 20%, 30%, 40%, 50% or greater decrease in tumor mass, or by altered (e.g., decreased with statistical significance) scan dimensions. In certain embodiments, the methods and therapeutic compositions described herein (for example, anti-HRS antibody, alone or in combination with cancer immunotherapy agent) are sufficient to result in stable disease. In certain embodiments, the methods and therapeutic compositions described herein (for example, anti-HRS antibody, alone or in combination with cancer immunotherapy agent) are sufficient to result in clinically relevant reduction in symptoms of a particular disease indication known to the skilled clinician.

In some embodiments, an anti-HRS antibody increases, complements, or otherwise enhances the anti-tumor and/or immunostimulatory activity of the cancer immunotherapy agent, relative to the cancer immunotherapy agent alone. In some embodiments, an anti-HRS antibody enhances the anti-tumor and/or immunostimulatory activity of the cancer immunotherapy agent by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to the cancer immunotherapy agent alone.

The methods and therapeutic compositions described herein can be used in the treatment of any variety of cancers or tumors. In some embodiments, the cancer is a primary cancer, i.e., a cancer growing at the anatomical site where tumor progression began and yielded a cancerous mass. In some embodiments, the cancer is a secondary or metastatic cancer, i.e., a cancer which has spread from the primary site or tissue of origin into one or more different sites or tissues. In some embodiments, the subject or patient has a cancer selected from one or more of melanoma (e.g., metastatic melanoma), pancreatic cancer, bone cancer, prostate cancer, small cell lung cancer, non-small cell lung cancer (NSCLC), mesothelioma, leukemia (e.g., lymphocytic leukemia, chronic myelogenous leukemia, acute myeloid leukemia, relapsed acute myeloid leukemia), lymphoma, hepatoma (hepatocellular carcinoma or HCC), sarcoma, B-cell malignancy, breast cancer (for example, estrogen receptor positive (ER+), estrogen receptor negative (ER−), Her2 positive (Her2+), Her2 negative (Her2−), or a combination thereof, e.g., ER+/Her2+, ER+/Her2−, ER−/Her2+, or ER−/Her2−; or “triple negative” breast cancer which is estrogen receptor-negative, progesterone receptor-negative, and HER2-negative), ovarian cancer, colorectal cancer, glioma (e.g., astrocytoma, oligodendroglioma, ependymoma, or a choroid plexus papilloma), glioblastoma multiforme (e.g., giant cell gliobastoma or a gliosarcoma), meningioma, pituitary adenoma, vestibular schwannoma, primary CNS lymphoma, primitive neuroectodermal tumor (medulloblastoma), kidney cancer (e.g., renal cell carcinoma), bladder cancer, uterine cancer, esophageal cancer, brain cancer, head and neck cancers, cervical cancer, testicular cancer, thyroid cancer, stomach cancer, virus-induced tumors such as, for example, papilloma virus-induced carcinomas (e.g., cervical carcinoma, cervical cancer), adenocarcinomas, herpes virus-induced tumors (e.g. Burkitt's lymphoma, EBV-induced B-cell lymphoma), hepatitis B-induced tumors (hepatocellular carcinomas), HTLV-1-indued and HTLV-2-induced lymphomas, acoustic neuroma, lung cancers (e.g., lung carcinoma, bronchial carcinoma), small-cell lung carcinomas, pharyngeal cancer, anal carcinoma, glioblastoma, rectal carcinoma, astrocytoma, brain tumors, retinoblastoma, basalioma, brain metastases, medulloblastomas, vaginal cancer, pancreatic cancer, testicular cancer, Hodgkin's syndrome, meningiomas, Schneeberger disease, hypophysis tumor, Mycosis fungoides, carcinoids, neurinoma, spinalioma, Burkitt's lymphoma, laryngeal cancer, renal cancer, thymoma, corpus carcinoma, bone cancer, non-Hodgkin's lymphomas, urethral cancer, CUP syndrome, head/neck tumors, oligodendroglioma, vulval cancer, intestinal cancer, colon carcinoma, oesophageal cancer (e.g., oesophageal carcinoma), wart involvement, tumors of the small intestine, craniopharyngeomas, ovarian carcinoma, genital tumors, ovarian cancer (e.g., ovarian carcinoma), pancreatic cancer (e.g., pancreatic carcinoma), endometrial carcinoma, liver metastases, penile cancer, tongue cancer, gall bladder cancer, leukaemia, plasmocytoma, and lid tumor.

In some embodiments, as noted above, the cancer or tumor is a metastatic cancer. Further to the above cancers, exemplary metastatic cancers include, without limitation, bladder cancers which have metastasized to the bone, liver, and/or lungs; breast cancers which have metastasized to the bone, brain, liver, and/or lungs; colorectal cancers which have metastasized to the liver, lungs, and/or peritoneum; kidney cancers which have metastasized to the adrenal glands, bone, brain, liver, and/or lungs; lung cancers which have metastasized to the adrenal glands, bone, brain, liver, and/or other lung sites; melanomas which have metastasized to the bone, brain, liver, lung, and/or skin/muscle; ovarian cancers which have metastasized to the liver, lung, and/or peritoneum; pancreatic cancers which have metastasized to the liver, lung, and/or peritoneum; prostate cancers which have metastasized to the adrenal glands, bone, liver, and/or lungs; stomach cancers which have metastasized to the liver, lung, and/or peritoneum; thyroid cancers which have metastasized to the bone, liver, and/or lungs; and uterine cancers which have metastasized to the bone, liver, lung, peritoneum, and/or vagina; among others.

In some embodiments, for example, where the cancer immunotherapy agent is a PD-1 or PD-L1 antagonist or inhibitor, the subject has one or more biomarkers (e.g., increased PD-1 or PD-L1 levels in cells such as cancer cells or cancer-specific CTLs) that make the suitable for PD-1 or PD-L1 inhibitor therapy. For instance, in some embodiments, the subject has increased fractions of programmed cell death 1 high/cytotoxic T lymphocyte-associated protein 4 high (e.g., PD-1^{hi}CTLA-4^{hi}) cells within a tumor-infiltrating CD8+ T cell subset (see, e.g., Daud et al., J Clin Invest. 126:3447-3452, 2016). As another example, in some embodiments, the subject has increased levels of Bim (B cell lymphoma 2-interacting (Bcl2-interacting) mediator) in circulating tumor-reactive (e.g., PD-1⁺CD11a^{hi}CD8⁺) T cells, and optionally has metastatic melanoma (see, e.g., Dronca et al., JCI Insight. May 5; 1(6): e86014, 2016).

Certain specific combinations include an anti-HRS antibody and a PD-L1 antagonist or inhibitor, for example, atezolizumab (MPDL3280A), avelumab (MSB0010718C), and durvalumab (MEDI4736), for treating a cancer selected from one or more of colorectal cancer, melanoma, breast cancer, non-small-cell lung carcinoma, bladder cancer, and renal cell carcinoma.

Some specific combinations include an anti-HRS antibody and a PD-1 antagonist, for example, nivolumab, for treating a cancer selected from one or more of Hodgkin's lymphoma, melanoma, non-small cell lung cancer, hepatocellular carcinoma, renal cell carcinoma, and ovarian cancer.

Particular specific combinations include an anti-HRS antibody and a PD-1 antagonist, for example, pembrolizumab, for treating a cancer selected from one or more of melanoma, non-small cell lung cancer, small cell lung cancer, head and neck cancer, and urothelial cancer.

Certain specific combinations include an anti-HRS antibody and a CTLA-4 antagonist, for example, ipilimumab and tremelimumab, for treating a cancer selected from one or more of melanoma, prostate cancer, lung cancer, and bladder cancer.

Some specific combinations include an anti-HRS antibody and an IDO antagonist, for example, indoximod (NLG-8189), 1-methyl-tryptophan (1MT), β-Carboline (norharmane; 9H-pyrido[3,4-b]indole), rosmarinic acid, or epacadostat, for treating a cancer selected from one or more of metastatic breast cancer and brain cancer optionally Glioblastoma Multiforme, glioma, gliosarcoma or malignant brain tumor.

Certain specific combinations include an anti-HRS antibody and the cytokine INF-α for treating melanoma, Kaposi sarcoma, and hematologic cancers. Also included is the combination of an anti-HRS antibody and IL-2 (e.g., Aldesleukin) for treating metastatic kidney cancer or metastatic melanoma.

Some specific combinations include an anti-HRS antibody and a T-cell based adoptive immunotherapy, for example, comprising CAR-modified T-cells targeted against CD-19, for treating hematological cancers such as acute lymphoblastic leukemia (ALL), chronic lymphocytic leukemia (CLL), and B-cell neoplasms (see, e.g., Maude et al., 2015, supra; Lorentzen and Straten, Scand J Immunol. 82:307-19, 2015; and Ramos et al., Cancer J. 20:112-118, 2014).

The methods for treating cancers can be combined with other therapeutic modalities. For example, a combination therapy described herein can be administered to a subject before, during, or after other therapeutic interventions, including symptomatic care, radiotherapy, surgery, transplantation, hormone therapy, photodynamic therapy, antibiotic therapy, or any combination thereof. Symptomatic care includes administration of corticosteroids, to reduce cerebral edema, headaches, cognitive dysfunction, and emesis, and administration of anti-convulsants, to reduce seizures. Radiotherapy includes whole-brain irradiation, fractionated radiotherapy, and radiosurgery, such as stereotactic radiosurgery, which can be further combined with traditional surgery.

Methods for identifying subjects with one or more of the diseases or conditions described herein are known in the art.

For in vivo use, as noted above, for the treatment of human or non-human mammalian disease or testing, the agents described herein are generally incorporated into one or more therapeutic or pharmaceutical compositions prior to administration, including veterinary therapeutic compositions.

Thus, certain embodiments relate to therapeutic compositions that comprise at least one antibody or antigen-binding fragment thereof that specifically binds to a human HRS polypeptide, as described herein. In some instances, a therapeutic or pharmaceutical composition comprises one or more of the agents described herein in combination with a pharmaceutically- or physiologically-acceptable carrier or excipient. Certain therapeutic compositions further comprise at least one cancer immunotherapy agent, as described herein.

Some therapeutic compositions comprise (and certain methods utilize) only one anti-HRS antibody or antigen-binding fragment thereof. Certain therapeutic compositions comprise (and certain methods utilize) a mixture of at least two, three, four, or five different anti-HRS antibodies or antigen-binding fragments thereof.

For instance, certain therapeutic compositions comprise at least two anti-HRS antibodies, including a first antibody or antigen-binding fragment thereof that specifically binds to at least one first epitope of a human HRS polypeptide, and a second antibody or antigen-binding fragment thereof that specifically binds to at least one second epitope of a human HRS polypeptide, wherein the at least one first epitope differs from the at least one second epitope. In some embodiments, the first and the second antibody or antigen-binding fragment thereof specifically and non-competitively bind to the same domain of the HRS polypeptide. In some instances, the first and the second antibody or antigen-binding fragment thereof specifically bind to the N-terminal domain, the aminoacylation domain, or the anticodon binding domain.

In some embodiments, the first and the second antibody or antigen-binding fragment thereof specifically and non-competitively bind to different domains of the HRS polypeptide. In some instances, the first antibody or antigen-binding fragment thereof specifically binds to the N-terminal domain, and the second antibody or antigen-binding fragment thereof specifically binds to the aminoacylation domain. In some embodiments, the first antibody or antigen-binding fragment thereof specifically binds to the N-terminal domain, and the second antibody or antigen-binding fragment thereof specifically binds to the anticodon binding domain. In some embodiments, the first antibody or antigen-binding fragment thereof specifically binds to the aminoacylation domain, and the second antibody or antigen-binding fragment thereof specifically binds to the anticodon binding

In some embodiments, the first and the second antibody or antigen-binding fragments thereof are both blocking antibodies. In some embodiments, the first and the second antibody or antigen-binding fragments thereof are both partial-blocking antibodies. In some instances, the first and the second antibodies or antigen-binding fragments thereof are both non-blocking antibodies.

In some instances, the first antibody or antigen-binding fragment thereof is a blocking antibody and the second antibody or antigen-binding fragment thereof is a partial-blocking antibody. In certain instances, the first antibody or antigen-binding fragment thereof is a blocking antibody and the second antibody or antigen-binding fragment thereof is a non-blocking antibody.

In some embodiments, the first and the second antibodies or antigen-binding fragments thereof both comprise an IgG Fc domain with high effector function in humans, for example, an IgG1 or IgG3 Fc domain. In some embodiments, the first and the second antibodies or antigen-binding fragments thereof comprise an IgG Fc domain with low effector function in humans, for example, an IgG2 or IgG4 Fc domain.

In some instances, the first antibody or antigen-binding fragment thereof comprises an IgG Fc domain with high effector function in humans, for example, an IgG1 or IgG3 Fc domain, and the second antibody or antigen-binding fragment thereof comprises an IgG Fc domain with low effector function in humans, for example, an IgG2 or IgG4 Fc domain.

In particular embodiments, the therapeutic composition comprising the agents such as antibodies or other polypeptide agents (e.g., anti-HRS antibodies) is substantially pure on a protein basis or a weight-weight basis, for example, the composition has a purity of at least about 80%, 85%, 90%, 95%, 98%, or 99% on a protein basis or a weight-weight basis.

In some embodiments, the antibodies (e.g., anti-HRS antibodies) or other polypeptide agents provided herein do not form aggregates, have a desired solubility, and/or have an immunogenicity profile that is suitable for use in humans, as described herein and known in the art. Thus, in some embodiments, the therapeutic composition comprising a polypeptide agent (for example, an antibody such as an anti-HRS antibody) is substantially aggregate-free. For example, certain compositions comprise less than about 10% (on a protein basis) high molecular weight aggregated proteins, or less than about 5% high molecular weight aggregated proteins, or less than about 4% high molecular weight aggregated proteins, or less than about 3% high molecular weight aggregated proteins, or less than about 2% high molecular weight aggregated proteins, or less than about 1% high molecular weight aggregated proteins. Some compositions comprise a polypeptide agent (e.g., an antibody such as an anti-HRS antibody) that is at least about 50%, about 60%, about 70%, about 80%, about 90% or about 95% monodisperse with respect to its apparent molecular mass.

In some embodiments, polypeptide agents such as antibodies (e.g., anti-HRS antibodies) are concentrated to about or at least about 0.1 mg/ml, 0.2 mg/ml, 0.3 mg/ml, 0.4 mg/ml, 0.5 mg/ml, 0.6, 0.7, 0.8, 0.9, 1 mg/ml, 2 mg/ml, 3 mg/ml, 4 mg/ml, 5 mg/ml, 6 mg/ml, 7 mg/ml, 8 mg/ml, 9 mg/ml, 10 mg/ml, 11, 12, 13, 14 or 15 mg/ml and are formulated for biotherapeutic uses.

To prepare a therapeutic or pharmaceutical composition, an effective or desired amount of one or more agents is mixed with any pharmaceutical carrier(s) or excipient known to those skilled in the art to be suitable for the particular agent and/or mode of administration. A pharmaceutical carrier may be liquid, semi-liquid or solid. Solutions or suspensions used for parenteral, intradermal, subcutaneous or topical application may include, for example, a sterile diluent (such as water), saline solution (e.g., phosphate buffered saline; PBS), fixed oil, polyethylene glycol, glycerin, propylene glycol or other synthetic solvent; antimicrobial agents (such as benzyl alcohol and methyl parabens); antioxidants (such as ascorbic acid and sodium bisulfite) and chelating agents (such as ethylenediaminetetraacetic acid (EDTA)); buffers (such as acetates, citrates and phosphates). If administered intravenously (e.g., by IV infusion), suitable carriers include physiological saline or phosphate buffered saline (PBS), and solutions containing thickening and solubilizing agents, such as glucose, polyethylene glycol, polypropylene glycol and mixtures thereof.

Administration of agents described herein, in pure form or in an appropriate therapeutic or pharmaceutical composition, can be carried out via any of the accepted modes of administration of agents for serving similar utilities. The therapeutic or pharmaceutical compositions can be prepared by combining an agent-containing composition with an appropriate physiologically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. In addition, other pharmaceutically active ingredients (including other small molecules as described elsewhere herein) and/or suitable excipients such as salts, buffers and stabilizers may, but need not, be present within the composition.

Administration may be achieved by a variety of different routes, including oral, parenteral, nasal, intravenous, intradermal, intramuscular, subcutaneous or topical. Preferred modes of administration depend upon the nature of the condition to be treated or prevented. Particular embodiments include administration by IV infusion.

Carriers can include, for example, pharmaceutically- or physiologically-acceptable carriers, excipients, or stabilizers that are non-toxic to the cell or mammal being exposed thereto at the dosages and concentrations employed. Often the physiologically-acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as polysorbate 20 (TWEEN™) polyethylene glycol (PEG), and poloxamers (PLURONICS™), and the like.

In some embodiments, one or more agents can be entrapped in microcapsules prepared, for example, by coacervation techniques or by interfacial polymerization (for example, hydroxymethylcellulose or gelatin-microcapsules and poly-(methylmethacylate)microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980). The particle(s) or liposomes may further comprise other therapeutic or diagnostic agents.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time, or may be divided into a number of smaller doses to be administered at intervals of time. For any particular subject, specific dosage regimens may be adjusted over time according to the individual need.

Typical routes of administering these and related therapeutic or pharmaceutical compositions thus include, without limitation, oral, topical, transdermal, inhalation, parenteral, sublingual, buccal, rectal, vaginal, and intranasal. The term parenteral as used herein includes subcutaneous injections, intravenous, intramuscular, intrasternal injection or infusion techniques. Therapeutic or pharmaceutical compositions according to certain embodiments of the present disclosure are formulated so as to allow the active ingredients contained therein to be bioavailable upon administration of the composition to a subject or patient. Compositions that will be administered to a subject or patient may take the form of one or more dosage units, where for example, a tablet may be a single dosage unit, and a container of a herein described agent in aerosol form may hold a plurality of dosage units. Actual methods of preparing such dosage forms are known, or will be apparent, to those skilled in this art; for example, see Remington: The Science and Practice of Pharmacy, 20th Edition (Philadelphia College of Pharmacy and Science, 2000). The composition to be administered will typically contain a therapeutically effective amount of an agent described herein, for treatment of a disease or condition of interest.

A therapeutic or pharmaceutical composition may be in the form of a solid or liquid. In one embodiment, the carrier(s) are particulate, so that the compositions are, for example, in tablet or powder form. The carrier(s) may be liquid, with the compositions being, for example, an oral oil, injectable liquid or an aerosol, which is useful in, for example, inhalatory administration. When intended for oral administration, the pharmaceutical composition is preferably in either solid or liquid form, where semi-solid, semi-liquid, suspension and gel forms are included within the forms considered herein as either solid or liquid. Certain embodiments include sterile, injectable solutions.

As a solid composition for oral administration, the pharmaceutical composition may be formulated into a powder, granule, compressed tablet, pill, capsule, chewing gum, wafer or the like. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent. When the pharmaceutical composition is in the form of a capsule, for example, a gelatin capsule, it may contain, in addition to materials of the above type, a liquid carrier such as polyethylene glycol or oil.

The therapeutic or pharmaceutical composition may be in the form of a liquid, for example, an elixir, syrup, solution, emulsion or suspension. The liquid may be for oral administration or for delivery by injection, as two examples. When intended for oral administration, preferred composition contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid therapeutic or pharmaceutical compositions, whether they be solutions, suspensions or other like form, may include one or more of the following adjuvants: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

A liquid therapeutic or pharmaceutical composition intended for either parenteral or oral administration should contain an amount of an agent such that a suitable dosage will be obtained. Typically, this amount is at least 0.01% of the agent of interest in the composition. When intended for oral administration, this amount may be varied to be between 0.1 and about 70% of the weight of the composition. Certain oral therapeutic or pharmaceutical compositions contain between about 4% and about 75% of the agent of interest. In certain embodiments, therapeutic or pharmaceutical compositions and preparations according to the present invention are prepared so that a parenteral dosage unit contains between 0.01 to 10% by weight of the agent of interest prior to dilution.

The therapeutic or pharmaceutical compositions may be intended for topical administration, in which case the carrier may suitably comprise a solution, emulsion, ointment or gel base. The base, for example, may comprise one or more of the following: petrolatum, lanolin, polyethylene glycols, bee wax, mineral oil, diluents such as water and alcohol, and emulsifiers and stabilizers. Thickening agents may be present in a therapeutic or pharmaceutical composition for topical administration. If intended for transdermal administration, the composition may include a transdermal patch or iontophoresis device.

The therapeutic or pharmaceutical compositions may be intended for rectal administration, in the form, for example, of a suppository, which will melt in the rectum and release the drug. The composition for rectal administration may contain an oleaginous base as a suitable nonirritating excipient. Such bases include, without limitation, lanolin, cocoa butter, and polyethylene glycol.

The therapeutic or pharmaceutical composition may include various materials, which modify the physical form of a solid or liquid dosage unit. For example, the composition may include materials that form a coating shell around the active ingredients. The materials that form the coating shell are typically inert, and may be selected from, for example, sugar, shellac, and other enteric coating agents. Alternatively, the active ingredients may be encased in a gelatin capsule. The therapeutic or pharmaceutical compositions in solid or liquid form may include a component that binds to agent and thereby assists in the delivery of the compound. Suitable components that may act in this capacity include monoclonal or polyclonal antibodies, one or more proteins or a liposome.

The therapeutic or pharmaceutical composition may consist essentially of dosage units that can be administered as an aerosol. The term aerosol is used to denote a variety of systems ranging from those of colloidal nature to systems consisting of pressurized packages. Delivery may be by a liquefied or compressed gas or by a suitable pump system that dispenses the active ingredients. Aerosols may be delivered in single phase, bi-phasic, or tri-phasic systems in order to deliver the active ingredient(s). Delivery of the aerosol includes the necessary container, activators, valves, subcontainers, and the like, which together may form a kit. One of ordinary skill in the art, without undue experimentation may determine preferred aerosols.

The compositions described herein may be prepared with carriers that protect the agents against rapid elimination from the body, such as time release formulations or coatings. Such carriers include controlled release formulations, such as, but not limited to, implants and microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

The therapeutic or pharmaceutical compositions may be prepared by methodology well known in the pharmaceutical art. For example, a therapeutic or pharmaceutical composition intended to be administered by injection may comprise one or more of salts, buffers and/or stabilizers, with sterile, distilled water so as to form a solution. A surfactant may be added to facilitate the formation of a homogeneous solution or suspension. Surfactants are compounds that non-covalently interact with the agent so as to facilitate dissolution or homogeneous suspension of the agent in the aqueous delivery system.

Also included are Intravenous Immunoglobulin IVIG preparations, which comprise one or more naturally-occurring anti-HRS antibodies, or antigen-binding fragments thereof, or polyclonal mixtures thereof (for example, enriched polyclonal mixtures and/or polyclonal mixtures from at least one or two or more donor subjects), as described herein. IVIG preparations comprising whole antibodies have been described for the treatment of certain autoimmune conditions, and can be prepared using established methodologies. (See, for example, U.S. Patent Application Nos. 2002/0114802; 2003/0099635; and US 2002/0098182).

Exemplary IVIG preparations can be obtained and prepared from donor serum or monoclonal or recombinant immunoglobulins, or other suitable blood derived fractions. In some embodiments, blood is collected from subjects that have been pre-screened to have significant titers of anti-Jo1 antibodies, and, for example, which are not taking immunosuppressive drugs or under immunosuppressive dosing regimens. Anti-Jo-1 antibody levels may be readily assessed using commercially available kits and/or clinical testing labs.

In some embodiments, a recombinant Jo-1 antigen (full-length HRS) is coupled covalently to polystyrene microspheres, which are impregnated with fluorescent dyes to create a unique fluorescent signature. Jo-1 antibodies, if present in diluted serum, bind to the Jo-1 antigen on the microspheres. The microspheres are washed to remove extraneous serum proteins. Phycoerythrin (PE)-conjugated antihuman IgG antibody, or other suitably fluorescently labeled detection antibody) can then be added to detect IgG anti-Jo-1 bound to the microspheres. The microspheres are washed to remove unbound conjugate, and bound conjugate is detected by laser photometry. A primary laser reveals the fluorescent signature of each microsphere to distinguish it from microspheres that are labeled with other antigens, and a secondary laser reveals the level of PE fluorescence associated with each microsphere. Results are calculated by comparing the median fluorescence response for Jo 1 microspheres to a 4-point calibration curve. (Package insert: Bioplex 2200 ANA Screen. Bio-Rad Laboratories, Hercules, Calif.).

In some embodiments, the blood is collected from the same species of animal (e.g., human) as the subject to which the immunoglobulin preparation will be administered (referred to as “homologous” immunoglobulins). The immunoglobulins are isolated from the blood by suitable procedures, such as, for example, Cohn fractionation, ultracentrifugation, electrophoretic preparation, ion exchange chromatography, affinity chromatography, immunoaffinity chromatography, polyethylene glycol fractionation, or the like. (See, e.g., Cohn et al., J. Am. Chem. Soc. 68:459-75 (1946); Oncley et al., J. Am. Chem. Soc. 71:541-50 (1949); Barundem et al., Vox Sang. 7:157-74 (1962); Koblet et al., Vox Sang. 13:93-102 (1967); U.S. Pat. Nos. 5,122,373 and 5,177,194).

In certain embodiments, an IVIG preparation is prepared from gamma globulin-containing products produced by the alcohol fractionation and/or ion exchange and affinity chromatography methods known to those skilled in the art. Purified Cohn Fraction II is commonly used. The starting Cohn Fraction II paste is typically about 95 percent IgG and is comprised of the four IgG subtypes. The different subtypes are present in Fraction II in approximately the same ratio as they are found in the pooled human plasma from which they are obtained. The Fraction II is further purified before formulation into an administrable product. For example, the Fraction II paste can be dissolved in a cold purified aqueous alcohol solution and impurities removed via precipitation and filtration. Following the final filtration, the immunoglobulin suspension can be dialyzed or diafiltered (for example, using ultrafiltration membranes having a nominal molecular weight limit of less than or equal to 100,000 daltons) to remove the alcohol. The solution can be concentrated or diluted to obtain the desired protein concentration and can be further purified by techniques well known to those skilled in the art.

In some embodiments, as above, the subject donors for an IVIG preparation are screened to ensure a serum or plasma anti-Jo-1 antibody content (e.g. anti-Jo-1 specific IgG level) of about or at least about 0.1 μg/mL, 0.2 μg/mL, 0.5 μg/mL, 1 μg/mL, 2 μg/mL, 5 μg/mL, 10 μg/mL, 20 μg/mL, 50 μg/mL, or 100 μg/mL. In certain embodiments, an IVIG preparation comprises one or more naturally-occurring anti-HRS antibodies at a concentration of about or at least about 1 μg/mL, 2 μg/mL, 5 μg/mL, 10 μg/mL, g/mL, 50 μg/mL, 100 μg/mL, 1 mg/mL, 2 mg/mL, 5 mg/mL, 10 mg/mL, or 100 mg/mL.

In certain embodiments, further preparative steps are employed to render an IVIG preparation safe for use in the methods described herein. Such steps can include, for example, treatment with solvent/detergent, pasteurization and sterilization.

In certain embodiments, an IVIG preparation is enriched with one or more recombinant ant-HARS antibodies. In some embodiments, an preparation of IVIG is supplemented with at least one recombinant anti-HRS antibody (described herein) to a total anti-HRS antibody concentration in the IVIG preparation of about or at least about 100 μg/mL, 1 mg/mL, 2 mg/mL, 5 mg/mL, 10 mg/mL, or 100 mg/mL.

In some embodiments, an IVIG preparation is enriched with or administered in combination with one or more additional therapeutic agents, including cancer immunotherapy agents, as described herein. Exemplary therapeutic agents, include for example, immune checkpoint modulatory agents, including antagonists or inhibitors of one or more inhibitory immune checkpoint molecules Exemplary inhibitory immune checkpoint molecules include for example Programmed Death-Ligand 1 (PD-L1), Programmed Death-Ligand 2 (PD-L2), Programmed Death 1 (PD-1), Cytotoxic T-Lymphocyte-Associated protein 4 (CTLA-4), Indoleamine 2,3-dioxygenase (IDO), tryptophan 2,3-dioxygenase (TDO), T-cell Immunoglobulin domain and Mucin domain 3 (TIM-3), Lymphocyte Activation Gene-3 (LAG-3), V-domain Ig suppressor of T cell activation (VISTA), B and T Lymphocyte Attenuator (BTLA), CD160, and T-cell immunoreceptor with Ig and ITIM domains (TIGIT).

The therapeutic or pharmaceutical or IVIG compositions may be administered in a therapeutically effective amount, which will vary depending upon a variety of factors including the activity of the specific compound employed; the metabolic stability and length of action of the compound; the age, body weight, general health, sex, and diet of the subject; the mode and time of administration; the rate of excretion; the drug combination; the severity of the particular disorder or condition; and the subject undergoing therapy. In some instances, a therapeutically effective daily dose is (for a 70 kg mammal) from about 0.001 mg/kg (i.e., ˜0.07 mg) to about 100 mg/kg (i.e., ˜7.0 g); preferably a therapeutically effective dose is (for a 70 kg mammal) from about 0.01 mg/kg (i.e., ˜0.7 mg) to about 50 mg/kg (i.e., 3.5 g); more preferably a therapeutically effective dose is (for a 70 kg mammal) from about 1 mg/kg (i.e., 70 mg) to about 25 mg/kg (i.e., ˜1.75 g). In some embodiments, the therapeutically effective dose is administered on a weekly, bi-weekly, or monthly basis. In specific embodiments, the therapeutically effective dose is administered on a weekly, bi-weekly, or monthly basis, for example, at a dose of about 1-10 or 1-5 mg/kg, or about 1, 2, 3, 4, 5, 6, 7, 8, 9, or 10 mg/kg.

The combination therapies described herein may include administration of a single pharmaceutical dosage formulation, which contains an anti-HRS antibody and an immunotherapy agent (optionally with one or more additional active agents), as well as administration of compositions comprising an anti-HRS antibody and a cancer immunotherapy agent in its own separate pharmaceutical dosage formulation. For example, an anti-HRS antibody as described herein and a cancer immunotherapy agent can be administered to the subject together in a single oral dosage composition such as a tablet or capsule, or each agent administered in separate oral dosage formulations. Similarly, an anti-HRS antibody as described herein and a cancer immunotherapy agent can be administered to the subject together in a single parenteral dosage composition such as in a saline solution or other physiologically acceptable solution, or each agent administered in separate parenteral dosage formulations. As another example, for cell-based therapies, an anti-HRS antibody can be mixed with the cells prior to administration, administered as part of a separate composition, or both. Where separate dosage formulations are used, the compositions can be administered at essentially the same time, i.e., concurrently, or at separately staggered times, i.e., sequentially and in any order; combination therapy is understood to include all these regimens.

Also included are patient care kits, comprising (a) at least one antibody or antigen-binding fragment thereof that specifically binds to a human histidyl-tRNA synthetase (HRS) polypeptide (an anti-HRS antibody); and (b) at least one cancer immunotherapy agent. In certain kits, (a) and (b) are in separate therapeutic compositions. In some kits, (a) and (b) are in the same therapeutic composition.

The kits herein may also include a one or more additional therapeutic agents or other components suitable or desired for the indication being treated, or for the desired diagnostic application. The kits herein can also include one or more syringes or other components necessary or desired to facilitate an intended mode of delivery (e.g., stents, implantable depots, etc.).

In some embodiments, a patient care kit contains separate containers, dividers, or compartments for the composition(s) and informational material(s). For example, the composition(s) can be contained in a bottle, vial, or syringe, and the informational material(s) can be contained in association with the container. In some embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of an anti-HRS antibody and optionally an immunotherapy agent. For example, the kit includes a plurality of syringes, ampules, foil packets, or blister packs, each containing a single unit dose of an anti-HRS antibody and optionally an immunotherapy agent. The containers of the kits can be air tight, waterproof (e.g., impermeable to changes in moisture or evaporation), and/or light-tight.

The patient care kit optionally includes a device suitable for administration of the composition, e.g., a syringe, inhalant, dropper (e.g., eye dropper), swab (e.g., a cotton swab or wooden swab), or any such delivery device. In some embodiments, the device is an implantable device that dispenses metered doses of the agent(s). Also included are methods of providing a kit, e.g., by combining the components described herein.

Bioassays and Analytical Assays for Drug Release Assays and Product Specifications, Diagnostics, and Reagents

Also included are bioassays that relate to anti-HRS antibodies and related agents such as therapeutic and diagnostic reagents. Examples include bioassays and analytical assays that measure purity, biological activity, affinity, solubility, pH, endotoxin levels, among others, many of which are described herein. Also included are assays that establish dose response curves and/or provide one or more bases for comparison between different batches of antibody. Batch comparisons can be based on any one or more of chemical characterization, biological characterization, and clinical characterization. Also included are methods of evaluating the potency, stability, pharmacokinetics, and immunogenicity of a selected antibody. Among other uses, these and other methods can be used for lot releasing testing of biologic or chemical agents, including anti-HRS antibodies, described herein.

Certain embodiments include the use of bioaffinity assays. Such assays can be used to assess the binding affinity, for example, between an anti-HRS antibody and its ability to modulate the interaction of a HRS polypeptide and a neuropilin 2 polypeptide, or other cellular binding partner, or between an HRS polypeptide and an anti-HRS antibody. Certain exemplary binding affinity assays may utilize ELISA assays, and other immunoassays as described herein and known in the art. Certain assays utilize high-performance receptor binding chromatography (see, e.g., Roswall et al., Biologicals. 24:25-39, 1996). Other exemplary binding affinity assays may utilize surface plasmon resonance (SPR)-based technologies. Examples include BIACore technologies, certain of which integrate SPR technology with a microfluidics system to monitor molecular interactions in real time at concentrations ranging from pM to mM. Also included are KINEXA™ assays, which provide accurate measurements of binding specificity, binding affinity, and binding kinetics/rate constants.

Certain embodiments relate to immunoassays for evaluating or optimizing the immunogenicity of anti-HRS antibodies. Examples include ex vivo human cellular assays and in vitro immuno-enzymatic assays to provide useful information on the immunogenic potential of a therapeutic protein. Ex vivo cell-response assays can be used, for example, to reproduce the cellular co-operation between antigen-presenting cells (APCs) and T-cells, and thereby measure T-cells activation after contact with a protein of interest. Certain in vitro enzymatic assays may utilize a collection of recombinant HLA-DR molecules that cover a significant portion of a relevant human population, and may include automated immuno-enzymatic assays for testing the binding of peptides (stemming from the fragmentation of the therapeutic protein) with the HLA-DR molecules. Also included are methods of reducing the immunogenicity of a selected protein, such as by using these and related methods to identify and then remove or alter one or more T-cell epitopes from an anti-HRS antibody.

Also included are biological release assays (e.g., cell-based assays) for measuring parameters such as specific biological activities, including non-canonical biological activities, and cytotoxicity. Certain specific biological assays include, for example, cell-based assays that utilize a cellular binding partner (e.g., cell-surface receptor (for example a neuropilin-2 peptide, or the full length Np-2 receptor, or HRS polypeptide presented on the cell surface), which is either endogenously, or recombinantly expressed on the cell surface), which is functionally coupled to a readout, such as a fluorescent or luminescent indicator of HRS polypeptide binding, or functional activity, as described herein. For instance, specific embodiments include a cell that comprises a neuropilin-2 cell-surface receptor or an extracellular portion thereof that binds to a HRS polypeptide, wherein the cell comprises a detector or readout, which enables the binding and/or activity of an anti-HRS antibody to modulate HRS polypeptide activity or binding to its cellular receptor to be assessed. Another embodiment include a cell that comprises a neuropilin-2 cell-surface receptor or an extracellular portion thereof that binds to a HRS polypeptide, wherein the HRS polypeptide comprises a detector or readout, which enables the binding and/or activity of an anti-HRS antibody to modulate HRS polypeptide activity or binding to its cellular receptor to be assessed. Some embodiments include a cell that comprises a neuropilin-2 cell-surface receptor or an extracellular portion thereof that binds to a HRS polypeptide, wherein an anti-HRS antibody comprises a detector or readout, which enables the binding and/or activity of an anti-HRS antibody to modulate HRS polypeptide activity or binding to its cellular receptor to be assessed. Certain embodiments includes a cell that either endogenously or recombinantly expresses and presents a HRS polypeptide on the cell surface, wherein an anti-HRS antibody comprises a detector or readout, which enables the binding and/or activity of an anti-HRS antibody to bind to the HRS polypeptide to be assessed.

Also included are in vivo biological assays to characterize the pharmacokinetics of an anti-HRS antibody, typically utilizing engineered, or wild type mice, rat, monkey or other mammal (see, e.g., Lee et al., The Journal of Pharmacology. 281:1431-1439, 1997). Examples of cytotoxicity-based biological assays include release assays (e.g., chromium or europium release assays to measure apoptosis; see, e.g., von Zons et al., Clin Diagn Lab Immunol. 4:202-207, 1997), among others, which can assess the cytotoxicity anti-HRS antibodies, whether for establishing dose response curves, batch testing, or other properties related to approval by various regulatory agencies, such as the Food and Drug Administration (FDA).

Also included are assays for evaluating the effects of an anti-HRS antibody on immune cells. Examples include an assay system, comprising an activated population of T-cells and at least anti-HRS antibody, wherein the anti-HRS antibody reduces extracellular signaling of extracellular HRS in vitro; and binds to at least two HRS splice variant polypeptides (see, e.g., Table H1) with an affinity of about or at least about 333 pM or tighter.

Certain embodiments include an assay system, comprising a single monoclonal anti-HRS antibody and an HRS polypeptide, wherein the anti-HRS antibody binds to HRS polypeptide, comprises an IgG4 Fc domain, and binds to at least two HRS splice variant polypeptides (see, e.g., Table H1) with an affinity of about or at least about 333 pM or tighter.

Also included are testing material(s), comprising a purified HRS polypeptide, wherein said purified HRS polypeptide is bound to a solid substrate in a manner that enables antibody binding detection.

Such assays and materials can be used, for example, to develop a dose response curve for a selected anti-HRS antibody, and/or to compare the dose response curve of different batches of proteins or other agents. A dose-response curve is an X-Y graph that relates the magnitude of a stressor to the response of a receptor, or receptor-HRS polypeptide interaction; the response may be a physiological or biochemical response, such as a non-canonical biological activity in a cell in vitro or in a cell or tissue in vivo, a therapeutically effective amount as measured in vivo (e.g., as measured by EC₅₀), or death, whether measured in vitro or in vivo (e.g., cell death, organismal death). Death is usually indicated as an LD₅₀, a statistically-derived dose that is lethal to 50% of a modeled population, though it can be indicated by LC₀₁ (lethal dose for 1% of the animal test population), LC₁₀₀ (lethal dose for 100% of the animal test population), or LC_{LO} (lowest dose causing lethality). Almost any desired effect or endpoint can be characterized in this manner.

The measured dose of a response curve is typically plotted on the X axis and the response is plotted on the Y axis. More typically, the logarithm of the dose is plotted on the X axis, most often generating a sigmoidal curve with the steepest portion in the middle. The No Observable Effect Level (NOEL) refers to the lowest experimental dose for which no measurable effect is observed, and the threshold dose refers to the first point along the graph that indicates a response above zero. As a general rule, stronger drugs generate steeper dose response curves. For many drugs, the desired effects are found at doses slightly greater than the threshold dose, often because lower doses are relatively ineffective and higher doses lead to undesired side effects. For in vivo generated dose response curves, a curve can be characterized by values such as g/kg, mg/kg, or g/kg of body-weight, if desired.

For batch comparisons, it can be useful to calculate the coefficient of variation (CV) between different dose response curves of different batches (e.g., between different batches of anti-HRS antibody), in part because the CV allows comparison between data sets with different units or different means. For instance, in certain exemplary embodiments, two or three or more different batches of anti-HRS antibodies or other agents have a CV between them of less than about 30%, 20%, 15%, 14%, 13%, 12%, 11%, 10%, 9%, 8%, 7%, 6%, 5%, 4%, 3%, 2%, or 1% for a 4, 5, 6, 7, or 8 point dose curve. In certain embodiments, the dose response curve is measured in a cell-based assay, and its readout relates to an increase or a decrease in a selected non-canonical activity of an anti-HRS antibody. In certain embodiments, the dose response curve is measured in a cell release assay or animal model (e.g., mouse model), and its readout relates to cell death or animal death. Other variations will be apparent to persons skilled in the art.

Expression and Purification Systems

Embodiments of the present invention include methods and related compositions for expressing and purifying an anti-HRS antibody or other polypeptide-based agent described herein. Such recombinant anti-HRS antibodies can be conveniently prepared using standard protocols as described for example in Sambrook, et al., (1989, supra), in particular Sections 16 and 17; Ausubel et al., (1994, supra), in particular Chapters 10 and 16; and Coligan et al., Current Protocols in Protein Science (John Wiley & Sons, Inc. 1995-1997), in particular Chapters 1, 5 and 6. As one general example, anti-HRS antibodies may be prepared by a procedure including one or more of the steps of: (a) preparing a construct comprising a polynucleotide sequences that encode an anti-HRS antibody heavy and light chain and that are operably linked to a regulatory element; (b) introducing the constructs into a host cell; (c) culturing the host cell to express an anti-HRS antibody; and (d) isolating an anti-HRS antibody from the host cell.

Anti-HRS antibody polynucleotides are described elsewhere herein. In order to express a desired polypeptide, a nucleotide sequence encoding an anti-HRS antibody, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (1989), and Ausubel et al., Current Protocols in Molecular Biology (1989).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems, including mammalian cell and more specifically human cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of β-galactosidase so that a hybrid protein is produced; plN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

Certain embodiments may employ E. coli-based expression systems (see, e.g., Structural Genomics Consortium et al., Nature Methods. 5:135-146, 2008). These and related embodiments may rely partially or totally on ligation-independent cloning (LIC) to produce a suitable expression vector. In specific embodiments, protein expression may be controlled by a T7 RNA polymerase (e.g., pET vector series). These and related embodiments may utilize the expression host strain BL21(DE3), a λDE3 lysogen of BL21 that supports T7-mediated expression and is deficient in lon and ompT proteases for improved target protein stability. Also included are expression host strains carrying plasmids encoding tRNAs rarely used in E. coli, such as ROSETTA™ (DE3) and Rosetta 2 (DE3) strains. Cell lysis and sample handling may also be improved using reagents sold under the trademarks BENZONASE® nuclease and BUGBUSTER® Protein Extraction Reagent. For cell culture, auto-inducing media can improve the efficiency of many expression systems, including high-throughput expression systems. Media of this type (e.g., OVERNIGHT EXPRESS™ Autoinduction System) gradually elicit protein expression through metabolic shift without the addition of artificial inducing agents such as IPTG. Particular embodiments employ hexahistidine tags (such as those sold under the trademark HIS⋅TAG® fusions), followed by immobilized metal affinity chromatography (IMAC) purification, or related techniques. In certain aspects, however, clinical grade proteins can be isolated from E. coli inclusion bodies, without or without the use of affinity tags (see, e.g., Shimp et al., Protein Expr Purif 50:58-67, 2006). As a further example, certain embodiments may employ a cold-shock induced E. coli high-yield production system, because over-expression of proteins in Escherichia coli at low temperature improves their solubility and stability (see, e.g., Qing et al., Nature Biotechnology. 22:877-882, 2004).

Also included are high-density bacterial fermentation systems. For example, high cell density cultivation of Ralstonia eutropha allows protein production at cell densities of over 150 g/L, and the expression of recombinant proteins at titers exceeding 10 g/L.

In the yeast Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987). Also included are Pichia pandoris expression systems (see, e.g., Li et al., Nature Biotechnology. 24, 210-215, 2006; and Hamilton et al., Science, 301:1244, 2003). Certain embodiments include yeast systems that are engineered to selectively glycosylate proteins, including yeast that have humanized N-glycosylation pathways, among others (see, e.g., Hamilton et al., Science. 313:1441-1443, 2006; Wildt et al., Nature Reviews Microbiol. 3:119-28, 2005; and Gerngross et al., Nature-Biotechnology. 22:1409-1414, 2004; U.S. Pat. Nos. 7,629,163; 7,326,681; and 7,029,872). Merely by way of example, recombinant yeast cultures can be grown in Fernbach Flasks or 15 L, 50 L, 100 L, and 200 L fermentors, among others.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et al., EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia cells. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia cells in which the polypeptide of interest may be expressed (Engelhard et al., Proc. Natl. Acad. Sci. U.S.A. 91:3224-3227 (1994)). Also included are baculovirus expression systems, including those that utilize SF9, SF21, and T. ni cells (see, e.g., Murphy and Piwnica-Worms, Curr Protoc Protein Sci. Chapter 5:Unit5.4, 2001). Insect systems can provide post-translation modifications that are similar to mammalian systems.

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Examples of useful mammalian host cell lines include monkey kidney CV1 line transformed by SV40 (COS-7, ATCC CRL 1651); human embryonic kidney line (293 or 293 cells sub-cloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TR1 cells (Mather et al., Annals N Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR-CHO cells (Urlaub et al., PNAS USA 77:4216 (1980)); and myeloma cell lines such as NSO and Sp2/0. For a review of certain mammalian host cell lines suitable for antibody production, see, e.g., Yazaki and Wu, Methods in Molecular Biology, Vol. 248 (B. K. C Lo, ed., Humana Press, Totowa, N.J., 2003), pp. 255-268. Certain preferred mammalian cell expression systems include CHO and HEK293-cell based expression systems. Mammalian expression systems can utilize attached cell lines, for example, in T-flasks, roller bottles, or cell factories, or suspension cultures, for example, in 1 L and 5 L spinners, 5 L, 14 L, 40 L, 100 L and 200 L stir tank bioreactors, or 20/50 L and 100/200 L WAVE bioreactors, among others known in the art.

Also included is the cell-free expression of proteins. These and related embodiments typically utilize purified RNA polymerase, ribosomes, tRNA and ribonucleotides; these reagents may be produced by extraction from cells or from a cell-based expression system.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results Probl. Cell Differ. 20:125-162 (1994)).

In addition, a host cell strain may be chosen for its ability to modulate the expression of the inserted sequences or to process the expressed protein in the desired fashion. Such modifications of the polypeptide include, but are not limited to, post-translational modifications such as acetylation, carboxylation, glycosylation, phosphorylation, lipidation, and acylation. Post-translational processing which cleaves a “prepro” form of the protein may also be used to facilitate correct insertion, folding and/or function. Different host cells such as yeast, CHO, HeLa, MDCK, HEK293, and W138, in addition to bacterial cells, which have or even lack specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for about 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type. Transient production, such as by transient transfection or infection, can also be employed. Exemplary mammalian expression systems that are suitable for transient production include HEK293 and CHO-based systems.

Any number of selection systems may be used to recover transformed or transduced cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk- or aprt-cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as green fluorescent protein (GFP) and other fluorescent proteins (e.g., RFP, YFP), anthocyanins, β-glucuronidase and its substrate GUS, and luciferase and its substrate luciferin, being widely used not only to identify transformants, but also to quantify the amount of transient or stable protein expression attributable to a specific vector system (see, e.g., Rhodes et al., Methods Mol. Biol. 55:121-131 (1995)).

Also included are high-throughput protein production systems, or micro-production systems. Certain aspects may utilize, for example, hexa-histidine fusion tags for protein expression and purification on metal chelate-modified slide surfaces or MagneHis Ni-Particles (see, e.g., Kwon et al., BMC Biotechnol. 9:72, 2009; and Lin et al., Methods Mol Biol. 498:129-41, 2009)). Also included are high-throughput cell-free protein expression systems (see, e.g., Sitaraman et al., Methods Mol Biol. 498:229-44, 2009). These and related embodiments can be used, for example, to generate microarrays of anti-HRS antibodies which can then be used for screening libraries to identify antibodies and antigen-binding domains that interact with the HRS polypeptide(s) of interest.

A variety of protocols for detecting and measuring the expression of polynucleotide-encoded products, using binding agents or antibodies such as polyclonal or monoclonal antibodies specific for the product, are known in the art. Examples include enzyme-linked immunosorbent assay (ELISA), western immunoblots, radioimmunoassays (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. Certain specific embodiments utilize serum free cell expression systems. Examples include HEK293 cells and CHO cells that can grown on serum free medium (see, e.g., Rosser et al., Protein Expr. Purif 40:237-43, 2005; and U.S. Pat. No. 6,210,922).

An antibody, or antigen-binding fragment thereof, produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification and/or detection of soluble proteins. Examples of such domains include cleavable and non-cleavable affinity purification and epitope tags such as avidin, FLAG tags, poly-histidine tags (e.g., 6×His), cMyc tags, V5-tags, glutathione S-transferase (GST) tags, and others.

The protein produced by a recombinant cell can be purified and characterized according to a variety of techniques known in the art. Exemplary systems for performing protein purification and analyzing protein purity include fast protein liquid chromatography (FPLC) (e.g., AKTA and Bio-Rad FPLC systems), high-pressure liquid chromatography (HPLC) (e.g., Beckman and Waters HPLC). Exemplary chemistries for purification include ion exchange chromatography (e.g., Q, S), size exclusion chromatography, salt gradients, affinity purification (e.g., Ni, Co, FLAG, maltose, glutathione, protein A/G), gel filtration, reverse-phase, ceramic HYPERD® ion exchange chromatography, and hydrophobic interaction columns (HIC), among others known in the art. Also included are analytical methods such as SDS-PAGE (e.g., coomassie, silver stain), immunoblot, Bradford, and ELISA, which may be utilized during any step of the production or purification process, typically to measure the purity of the protein composition.

Also included are methods of concentrating anti-HRS antibodies and antigen binding fragments thereof, and composition comprising concentrated soluble proteins. In different aspects such concentrated solutions of anti-HRS antibodies may comprise proteins at a concentration of about 5 mg/mL; or about 8 mg/mL; or about 10 mg/mL; about 15 mg/mL; or about 20 mg/mL.

In some aspects, such compositions may be substantially monodisperse, meaning that an at least one anti-HRS antibody exists primarily (i.e. at least about 90%, or greater) in one apparent molecular weight form when assessed for example, by size exclusion chromatography, dynamic light scattering, or analytical ultracentrifugation.

In some aspects, such compositions have a purity (on a protein basis) of at least about 90%, or in some aspects at least about 95% purity, or in some embodiments, at least 98% purity. Purity may be determined via any routine analytical method as known in the art.

In some aspects, such compositions have a high molecular weight aggregate content of less than about 10%, compared to the total amount of protein present, or in some embodiments such compositions have a high molecular weight aggregate content of less than about 5%, or in some aspects such compositions have a high molecular weight aggregate content of less than about 3%, or in some embodiments a high molecular weight aggregate content of less than about 1%. High molecular weight aggregate content may be determined via a variety of analytical techniques including for example, by size exclusion chromatography, dynamic light scattering, or analytical ultracentrifugation.

Examples of concentration approaches contemplated herein include lyophilization, which is typically employed when the solution contains few soluble components other than the protein of interest. Lyophilization is often performed after HPLC run, and can remove most or all volatile components from the mixture. Also included are ultrafiltration techniques, which typically employ one or more selective permeable membranes to concentrate a protein solution. The membrane allows water and small molecules to pass through and retains the protein; the solution can be forced against the membrane by mechanical pump, gas pressure, or centrifugation, among other techniques.

In certain embodiments, the reagents, anti-HRS antibodies, or related agents have a purity of at least about 90%, as measured according to routine techniques in the art. In certain embodiments, such as diagnostic compositions or certain therapeutic compositions, an anti-HRS antibody composition has a purity of at least about 95%. In specific embodiments, such as therapeutic or pharmaceutical compositions, an anti-HRS antibody composition has a purity of at least about 97% or 98% or 99%. In other embodiments, such as when being used as reference or research reagents, anti-HRS antibodies can be of lesser purity, and may have a purity of at least about 50%, 60%, 70%, or 80%. Purity can be measured overall or in relation to selected components, such as other proteins, e.g., purity on a protein basis.

Purified anti-HRS antibodies can also be characterized according to their biological characteristics. Examples include binding affinity or binding kinetics to a selected ligand (e.g., a cellular binding partner of an anti-HRS antibody, or the interaction of that receptor (e.g. HRS polypeptide) with a cell-surface receptor (e.g. neuropilin 2) or an extracellular domain thereof (e.g. Np2-fc fusion protein. Binding affinity and binding kinetics can be measured according to a variety of techniques known in the art, such as Biacore® and related technologies that utilize surface plasmon resonance (SPR), an optical phenomenon that enables detection of unlabeled interactants in real time. SPR-based biosensors can be used in determination of active concentration, screening and characterization in terms of both affinity and kinetics. The presence or levels of one or more canonical or non-canonical biological activities can be measured according to cell-based assays, including those that utilize a cellular binding partner (e.g., cell-surface receptor, such as surface presented, or HRS polypeptides in free solution, or cell bound or soluble neuropilin-2) of a selected anti-HRS antibody, which is functionally coupled to a readout or indicator, such as a fluorescent or luminescent indicator of biological activity, as described herein.

In certain embodiments, as noted above, an anti-HRS antibody composition is substantially endotoxin free, including, for example, about 95% endotoxin free, preferably about 99% endotoxin free, and more preferably about 99.99% endotoxin free. The presence of endotoxins can be detected according to routine techniques in the art, as described herein. In specific embodiments, an anti-HRS antibody composition is made from a eukaryotic cell such as a mammalian or human cell in substantially serum free media. In certain embodiments, as noted herein, an anti-HRS antibody composition has an endotoxin content of less than about 10 EU/mg of anti-HRS antibody, or less than about 5 EU/mg of anti-HRS antibody, less than about 3 EU/mg of anti-HRS antibody, or less than about 1 EU/mg of anti-HRS antibody.

In certain embodiments, an anti-HRS antibody composition comprises less than about 10% wt/wt high molecular weight aggregates, or less than about 5% wt/wt high molecular weight aggregates, or less than about 2% wt/wt high molecular weight aggregates, or less than about or less than about 1% wt/wt high molecular weight aggregates.

Also included are protein-based analytical assays and methods, which can be used to assess, for example, protein purity, size, solubility, and degree of aggregation, among other characteristics. Protein purity can be assessed a number of ways. For instance, purity can be assessed based on primary structure, higher order structure, size, charge, hydrophobicity, and glycosylation. Examples of methods for assessing primary structure include N- and C-terminal sequencing and peptide-mapping (see, e.g., Allen et al., Biologicals. 24:255-275, 1996)). Examples of methods for assessing higher order structure include circular dichroism (see, e.g., Kelly et al., Biochim Biophys Acta. 1751:119-139, 2005), fluorescent spectroscopy (see, e.g., Meagher et al., J. Biol. Chem. 273:23283-89, 1998), FT-IR, amide hydrogen-deuterium exchange kinetics, differential scanning calorimetry, NMR spectroscopy, immunoreactivity with conformationally sensitive antibodies. Higher order structure can also be assessed as a function of a variety of parameters such as pH, temperature, or added salts. Examples of methods for assessing protein characteristics such as size include analytical ultracentrifugation and size exclusion HPLC (SEC-HPLC), and exemplary methods for measuring charge include ion-exchange chromatography and isolectric focusing. Hydrophobicity can be assessed, for example, by reverse-phase HPLC and hydrophobic interaction chromatography HPLC. Glycosylation can affect pharmacokinetics (e.g., clearance), conformation or stability, receptor binding, and protein function, and can be assessed, for example, by mass spectrometry and nuclear magnetic resonance (NMR) spectroscopy.

As noted above, certain embodiments include the use of SEC-HPLC to assess protein characteristics such as purity, size (e.g., size homogeneity) or degree of aggregation, and/or to purify proteins, among other uses. SEC, also including gel-filtration chromatography (GFC) and gel-permeation chromatography (GPC), refers to a chromatographic method in which molecules in solution are separated in a porous material based on their size, or more specifically their hydrodynamic volume, diffusion coefficient, and/or surface properties. The process is generally used to separate biological molecules, and to determine molecular weights and molecular weight distributions of polymers. Typically, a biological or protein sample (such as a protein extract produced according to the protein expression methods provided herein and known in the art) is loaded into a selected size-exclusion column with a defined stationary phase (the porous material), preferably a phase that does not interact with the proteins in the sample. In certain aspects, the stationary phase is composed of inert particles packed into a dense three-dimensional matrix within a glass or steel column. The mobile phase can be pure water, an aqueous buffer, an organic solvent, or a mixture thereof. The stationary-phase particles typically have small pores and/or channels which only allow molecules below a certain size to enter. Large particles are therefore excluded from these pores and channels, and their limited interaction with the stationary phase leads them to elute as a “totally-excluded” peak at the beginning of the experiment. Smaller molecules, which can fit into the pores, are removed from the flowing mobile phase, and the time they spend immobilized in the stationary-phase pores depends, in part, on how far into the pores they penetrate. Their removal from the mobile phase flow causes them to take longer to elute from the column and results in a separation between the particles based on differences in their size. A given size exclusion column has a range of molecular weights that can be separated. Overall, molecules larger than the upper limit will not be trapped by the stationary phase, molecules smaller than the lower limit will completely enter the solid phase and elute as a single band, and molecules within the range will elute at different rates, defined by their properties such as hydrodynamic volume. For examples of these methods in practice with pharmaceutical proteins, see Bruner et al., Journal of Pharmaceutical and Biomedical Analysis. 15: 1929-1935, 1997.

Protein purity for clinical applications is also discussed, for example, by Anicetti et al. (Trends in Biotechnology. 7:342-349, 1989). More recent techniques for analyzing protein purity include, without limitation, the LabChip GXII, an automated platform for rapid analysis of proteins and nucleic acids, which provides high throughput analysis of titer, sizing, and purity analysis of proteins. In certain non-limiting embodiments, clinical grade proteins such as protein fragments and antibodies can be obtained by utilizing a combination of chromatographic materials in at least two orthogonal steps, among other methods (see, e.g., Therapeutic Proteins: Methods and Protocols. Vol. 308, Eds., Smales and James, Humana Press Inc., 2005). Typically, protein agents (e.g., anti-HRS antibodies, and antigen-binding fragments) are substantially endotoxin-free, as measured according to techniques known in the art and described herein.

Protein solubility assays are also included. Such assays can be utilized, for example, to determine optimal growth and purification conditions for recombinant production, to optimize the choice of buffer(s), and to optimize the choice of Anti-HRS antibodies or variants thereof. Solubility or aggregation can be evaluated according to a variety of parameters, including temperature, pH, salts, and the presence or absence of other additives. Examples of solubility screening assays include, without limitation, microplate-based methods of measuring protein solubility using turbidity or other measure as an end point, high-throughput assays for analysis of the solubility of purified recombinant proteins (see, e.g., Stenvall et al., Biochim Biophys Acta. 1752:6-10, 2005), assays that use structural complementation of a genetic marker protein to monitor and measure protein folding and solubility in vivo (see, e.g., Wigley et al., Nature Biotechnology. 19:131-136, 2001), and electrochemical screening of recombinant protein solubility in Escherichia coli using scanning electrochemical microscopy (SECM) (see, e.g., Nagamine et al., Biotechnology and Bioengineering. 96:1008-1013, 2006), among others. Anti-HRS antibodies with increased solubility (or reduced aggregation) can be identified or selected for according to routine techniques in the art, including simple in vivo assays for protein solubility (see, e.g., Maxwell et al., Protein Sci. 8:1908-11, 1999).

Protein solubility and aggregation can also be measured by dynamic light scattering techniques. Aggregation is a general term that encompasses several types of interactions or characteristics, including soluble/insoluble, covalent/noncovalent, reversible/irreversible, and native/denatured interactions and characteristics. For protein therapeutics, the presence of aggregates is typically considered undesirable because of the concern that aggregates may cause an immunogenic reaction (e.g., small aggregates), or may cause adverse events on administration (e.g., particulates). Dynamic light scattering refers to a technique that can be used to determine the size distribution profile of small particles in suspension or polymers such as proteins in solution. This technique, also referred to as photon correlation spectroscopy (PCS) or quasi-elastic light scattering (QELS), uses scattered light to measure the rate of diffusion of the protein particles. Fluctuations of the scattering intensity can be observed due to the Brownian motion of the molecules and particles in solution. This motion data can be conventionally processed to derive a size distribution for the sample, wherein the size is given by the Stokes radius or hydrodynamic radius of the protein particle. The hydrodynamic size depends on both mass and shape (conformation). Dynamic scattering can detect the presence of very small amounts of aggregated protein (<0.01% by weight), even in samples that contain a large range of masses. It can also be used to compare the stability of different formulations, including, for example, applications that rely on real-time monitoring of changes at elevated temperatures. Accordingly, certain embodiments include the use of dynamic light scattering to analyze the solubility and/or presence of aggregates in a sample that contains an anti-HRS antibody of the invention.

All publications, patent applications, and issued patents cited in this specification are herein incorporated by reference as if each individual publication, patent application, or issued patent were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Materials and Methods

Test Antibodies and Other Reagents for Animal Studies.
Anti-HRS C-terminal antibody: ATYR13C8 (13C8) (Mouse IgG1 kappa) stored in PBS, pH 7.4.
Anti-HRS N-terminal antibody: ATYR13E9 (13E9) (Mouse IgG1 kappa) stored in PBS, pH 7.4;
Anti-HRS N-terminal antibody: KL31-600 (human IgG1 kappa) stored in PBS, pH 7.4;
Anti-HRS N-terminal antibody: KL31-241 (human IgG1 kappa) stored in PBS, pH 7.4;
Anti-PD1 antibody (αPD1, or αmPD1): Clone RMP1-14, (Rat IgG2a), stored in PBS, pH 7, Bio X Cell Cat. No. BE0146.
Anti-PD-L1 antibody (αPD-L1, or αmPD-L1): Clone 10F.9G2, (Rat IgG2b) stored in PBS, pH 6.5, Bio X Cell Cat No. BE0101.
Anti-CTLA-4 antibody (αCTLA-4): Clone UC10-4F10-11, (Armenian Hamster IgG) stored in PBS, pH 6.5, Bio X Cell Cat No. BE0032.
Anti-CD4 antibody (αmCD4): Clone GK1.5 (rat IgG2b) stored in PBS, pH 7, Bio X Cell Cat. No. BE003
Anti-NK1.1 antibody (αmNK1.1): Clone PK136 (mouse IgG2a) stored in PBS, pH 7, Bio X Cell Cat. No. BE0036
Anti-CD8a antibody (αmCD8): Clone 2.43 (rat IgG2b) stored in PBS, pH 7, Bio X Cell Cat. No. BE0061
Control IgG1 antibody: Clone MOPC-21, (Mouse IgG1), stored in PBS, pH 6.5, Bio X Cell Cat No. BE0083.
Control IgG2a antibody, Clone 2A3, (Rat IgG2a), stored in PBS, pH 7.0 Bio X Cell Cat. No. BE0089.
Control IgG2b antibody, Clone LTF-2, (Rat IgG2b), stored in PBS, pH 7.0 Bio X Cell BE0090.

Antibodies were dosed intraperitoneally at a volume of less than 10 mL/kg unless noted otherwise.

Indole 2, 3-Dioxygenase-1 Inhibitor
Epacadostat (INCB024360) a small molecule IDO inhibitor (IDOi), was obtained from BPS Biosciences (catalog #27339-1). The IDOi was administered twice daily via oral gavage at a dose of 100 mg/kg twice daily in a vehicle of 3% N,N-Dimethylacetamide, 10% (2-Hydroxypropyl) 3-Cyclodextrin.

Antibody Generation.

Murine monoclonal antibodies to human histidyl-tRNA synthetase (HARS or HRS) were produced at The Scripps Research Institute (TSRI) Center for Antibody Development and Production. The anti-HRS antibody ATYR13C8 was generated by immunizing mice with recombinant protein representing residues 61-506 of human HARS (Lot H-I2-V5H-340). The anti-HRS antibody ATYR13E9 was generated by immunizing mice with recombinant protein representing residues 1-60 of mouse HARS (Lot muH-N4-061). For both projects, spleens were isolated from immunized animals and fusion with mouse myeloma cells was performed to generate hybridomas using standard techniques. Fusion, plating into 96-well plates, ELISA screening of hybridomas, expansion and characterization of positive hybridomas (titer and isotype) and freezing of up to 15 hybridomas per antigen, as well as 2-3 rounds of subcloning per hybridoma was performed at TSRI.

Production of large batches of 13C8 and 13E9 to provide protein amounts sufficient for in vivo work was done in 0.8 to 1.2 L flasks in which hybridoma cells were expanded and cultured over several weeks. Antibodies were purified from conditioned medium starting at 2 weeks of culture by flowing over a Protein A affinity column, eluting and storing in Phosphate Buffered Saline (1×PBS), pH 7.4. Each lot was tested for protein concentration, purity and endotoxin level. Purity by SDS-PAGE was routinely >90%.

Human anti-HRS antibodies were cloned from B cells obtained from the serum of individual donors who had been diagnosed as Jo-1 positive, in collaboration with AbCellera Biologics Inc, (Vancouver, BC V6T 1Z4, Canada) using microfabricated plates to select single antibody secreting cells using fluorescent beads coated with HRS polypeptides, and single cell sequencing. Essentially as described in PCT/CA2016/000031, and Kaston Leung et al., Proc Natl Acad Sci USA. 2016 Jul. 26; 113(30): 8484-8489. Humanized murine monoclonal antibodies were prepared via the selective replacement of murine antibody sequences compared with the corresponding sequences from a human antibody, and screening to confirm binding to HRS polypeptides. Both fully human and humanized antibodies were affinity matured via the systematic mutation of their CDR sequences, and the recombination of mutations in the higher affinity clones was identified.

Animals and Animal Husbandry.

All mice were ear tagged for identification purpose. Upon arrival, animals were examined to ensure that they were healthy. The animals were housed in autoclaved solid floor polycarbonate cages. Housing and sanitation were performed according to industry standards. All animal handling was performed in a laminar flow hood located in a clean room. In all experiments, euthanasia criteria were set for an upper limit on subcutaneous tumor volume (usually >2,000 mm³) and body weight loss (usually >20% body weight loss for an individual during the study). Animal work was performed at either Washington Biotechnology, Inc. 6200 Seaforth Street, Baltimore, Md. 21224, or at Crown Bioscience San Diego, 11011Torreyana Road, San Diego, Calif. 92121, USA; or at aTyr Pharma, 3545 John Hopkins Court, Suite 250, San Diego, Calif. 92121.

Cell Culture and Implantation.

Mouse B16-F10 cell line was purchased from ATCC. The cells were cultured in 75 cm² flasks containing DMEM media supplemented with 10% fetal bovine serum (FBS) and incubated at 37° C. in humidified atmosphere of 5% CO₂. As cells reached 90% confluence, cultures were expanded to 175 cm² flasks until sufficient cells are available for injection. 10,000 cancer cells in PBS with 20% matrigel (Examples 2-4, 6, 10, 14-16) were subcutaneously into the right flank of each mouse The day on which the tumor was implanted is designated as Day 0.

Mouse 4T1 cell line was purchased from ATCC. The cells were cultured in 75 cm² flasks containing DMEM media supplemented with 10% FBS and incubated at 37° C. in humidified atmosphere of 5% CO₂. As cells reached 90% confluence, cultures were expanded to 150 cm² flasks until sufficient cells are available for injection. 50,000 cancer cells in PBS with 20% matrigel were implanted subcutaneously into the right flank of each mouse (Examples 7, 11).

Human A549 cell line was purchased from ATCC. The cells were thawed and cultured in 75 cm² flasks containing DMEM media supplemented with 10% FBS, L-glutamine and penicillin/streptomycin and incubated at 37° C. in humidified atmosphere of 5% CO₂. As cells reached 90% confluence, cultures were expanded to 150 cm² flasks until sufficient cells are available for injection. 10,000,000 or 2,000,000 cancer cells in PBS with 20% matrigel were subcutaneously implanted into right flank of each mouse (Example 8).

Mouse CT26 cell line was purchased from ATCC. The cells were cultured in 75 cm² flasks containing RPMI-1640 media supplemented with 10% FBS and incubated at 37° C. in humidified atmosphere of 5% CO₂. As cells reached 90% confluence, cultures were expanded to 175 cm² flasks until sufficient cells are available for injection. 20,000 cancer cells in PBS with 20% matrigel were subcutaneously into right flank of each mouse (Examples 9, 13, and 17).

Mouse Pan02 cell line was obtained from a commercially available source. The cells were cultured in 75 cm² flasks containing DMEM media supplemented with 10% FBS and incubated at 37° C. in humidified atmosphere of 5% CO₂. As cells reached 90% confluence, cultures were expanded to 175 cm² flasks until sufficient cells are available for injection. 50,000 cancer cells in PBS with 20% matrigel were subcutaneously into right flank of each mouse (Example 12).

Tumor Monitoring.

Once palpable, sub-cutaneous tumors were measured three times a week with a digital caliper.

Tumor volumes were calculated using formula:

Tumor Volume=length×width×width×½

Tumor Antigen Exposure Protocol.

Mice were divided into two pre-treatment groups for conditioning. Thirty animals were implanted on the right flank with B16F10 cells as described above (tumor exposed) while the remaining animals were not implanted (tumor naive). Tumorectomy was performed to remove the tumor when tumor volumes averaged ˜100 mm³. After recovery, all animals were implanted on the left flank with B16F10 cells (designated as Day 0).

Depletion of Immune Cell Populations.

Depleting antibodies specific to mouse immune cells (CD4+ T cells, CD8+ T cells and NK1.1+ natural killer cells) were administered at a dose of 100 μg/mouse IP twice weekly beginning the day before tumor inoculation. To confirm depletion, mouse blood obtained by cheek venipuncture was incubated with Mouse BD FcBlock at 1:500 (BD Pharmingen #553141) for 15 min at room temperature. Samples were then stained with antibodies detecting NK cells (NK1.1-AF647, clone PK136 at 1:100, BioLegend #108708), T cells (CD3-PE, clone 17A2 at 1:200, BioLegend #100205), CD4+ cells (CD4-PE, clone RM4-5 at 1:200, Tonbo #25-0042-U100), and CD8+ cells ((CD8-FITC, clone 53-6.7 at 1:200, BioLegend #100706) for approximately 30 min at room temperature. Red blood cells were lysed by addition of 1-step Fix/Lyse Solution (Thermo Scientific #00-5333-54) and incubation for 15 min at room temperature. Cells were then pelleted at 400 g for 7 minutes, washed with FWB buffer (PBS/2% FBS) and pelleted again, and resuspended in 200 μL FWB buffer. Samples were acquired on a MACSQuant Analyzer flow cytometer (Miltenyi), and the lymphocyte population was gated on forward/side scatter plots. Within the lymphocyte gate, the percentages of NK1.1+/CD3− cells (NK cells), CD4+/CD8− cells (CD4+ T cells) and CD8+/CD4− cells (CD8+ T cells) were determined NOD mouse model of type 1 diabetes induction. Female NOD/ShiLtJ mice (Jax catalog #001976), which are prone to the development of auto-immune type 1 diabetes, arrived at 7 weeks of age and were placed on study at approximately 11 weeks of age. Prior to the commencement of antibody treatments, an intraperitoneal glucose tolerance test was performed and a fed glucose measurement was obtained using a handheld glucometer designed for rodent sampling (Alphatrak). Anti-mouse PD-L1 antibody or anti-HRS antibodies were administered twice weekly and glucose levels were measured using a glucometer during the 2 weeks of antibody administration. Diabetes was defined by a glucometer reading over 250 mg/dL. Animals were euthanized when glucose levels exceeded the maximum value reported by the glucometer (750 mg/dL) or animals were moribund.

ELISA Assays

Assays to Measure Human and Mouse Endogenous HRS in Circulation.

ELISA detection assays were developed to quantitate the levels of mouse or human HRS in circulation using different capture and detection antibodies to enable selective measurement of full length HRS as well as the N-terminal region.

The Human N-terminal ELISA is designed to detect the N-terminal domain of human HRS (WHEP domain) utilizing capture and detection antibodies targeting this domain (approximately amino acids 1-60 of HRS).

ELISA assays were conducted using a 96 well Multi-Array plate coated with capture antibody, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibody: ATYR12H6, mouse monoclonal antibody
Capture antibody conc: 1 μg/mL
Protein standard range: 100-0.046 ng/mL
Detection antibody: 1C8-b, biotinylated mouse monoclonal antibody,
Detection antibody conc: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant, #R92TC-1

The Human full-length HRS ELISA is designed to detect the multi-domain human HARS utilizing capture and detection antibodies targeting separate domains of the protein (WHEP (amino acids 1-60 of HRS) and catalytic domains (approximately amino acids 60-398 of HRS).

ELISA assays were conducted using a 96 well Multi-Array plate coated with capture antibody, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibody: ATYR12H6, mouse monoclonal antibody
Capture antibody conc: 1 μg/mL
Protein standard range: 100-0.046 ng/mL
Detection antibody: ATYR13C8-b, biotinylated mouse monoclonal antibody
Detection antibody conc: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant, #R92TC-1

The Mouse N-terminal ELISA is designed to detect the N-terminal domain of mouse HRS (WHEP) utilizing capture and detection antibodies targeting this domain.

ELISA assays were conducted using a 96 well Multi-Array plate coated with capture antibody, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibody: ATYR13E9, mouse monoclonal antibody
Capture antibody conc: 1 μg/mL
Protein standard range: 100-0.046 ng/mL
Detection antibody: 1C8-b, biotinylated mouse monoclonal antibody,
Detection antibody conc: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant, #R92TC-1

The Mouse full-length HRS ELISA designed to detect the multi-domain mouse HARS utilizing capture and detection antibodies targeting separate domains of the protein (WHEP (approximately amino acids 1-60 of HRS) and catalytic domains (approximately amino acids 60-398 of HRS).

ELISA assays were conducted using a 96 well Multi-Array plate coated with capture antibody, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibody: ATYR13E9, mouse monoclonal
Capture antibody conc: 1 μg/mL
Protein standard range: 100-0.046 ng/mL
Detection antibody: ATYR13C8-b, mouse monoclonal
Detection antibody conc: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant, #R92TC-1

Assays to Measure Pharmacokinetic Properties of ATYR13E9 and ATYR13C8 Monoclonal Antibodies.

ELISA assays were conducted using a 96 well Multi-Array plate coated with mouse HRS, following standard Meso Scale Discovery ELISA protocols, and using the following reagents:
Block buffer: Casein (Thermo Scientific #37528)
Wash buffer: PBS-T (0.05% Tween-20 in 1×PBS)
Diluent: 1% BSA diluted in PBS
Capture protein: Mouse HRS (mHARS-XH-258)
Capture protein conc: 2 μg/mL
Protein standard range: 100-0.14 ng/mL
Detection antibodies: Goat anti-mouse SULFO tag (Meso Scale Diagnostics Cat.#R32AC-1)
Detection antibody conc: 1 μg/mL
Substrate: Read Buffer T (4×) with Surfactant (Meso Scale Diagnostics Cat.#R92TC-1)

Assays to Measure Human Endogenous Neuropilin 2 (NRP2, or NP2) in Circulation.

An ELISA detection assay was developed to quantitate the levels of human NRP2 in circulation using capture and detection antibodies to enable selective measurement of soluble NRP2. The human NRP2 ELISA was designed to detect soluble NRP2 utilizing a monoclonal capture antibody and a polyclonal detection antibody targeting NRP2. ELISA assays were conducted using a 96 well Multi-Array plate, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibody: NRP2 mAb Cat.#MAB2215, R&D Systems
Capture antibody conc: 2 μg/mL
Protein standard: NRP2 Fc Cat.#2215-N2-025, R&D Systems
Protein standard range: 100-0.046 ng/mL
Detection antibody: NRP2 pAb Cat.#BAF2215, R&D Systems
Detection antibody cone: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant #R92TC-1

Assays to Measure Human Endogenous HRS & NRP-2 Complexes in Circulation.

An ELISA detection assay was developed to measure levels of human HRS & NRP2 complexes in circulation using capture and detection antibodies specific to each protein partner. The human HRS & NRP-2 complex ELISA is designed to detect a complex of soluble NRP2 and HRS utilizing monoclonal and polyclonal antibodies specific for these two proteins. ELISA assays were conducted using a 96 well Multi-Array plate, following standard Meso Scale Diagnostics ELISA protocols, and using the following reagents:
Block buffer: Casein (Thermo Scientific #37528)
Wash buffer: PBST (0.05% Tween-20 in 1×PBS; made in-house)
Diluent: 1% BSA (diluted in PBS) and Casein
Capture antibodies: NRP2 mAb Cat.#MAB22151, R&D Systems
   HRS C-terminal mAb Clone#ATYR13C8
   HRS N-terminal mAb Clone #ATYR12H6
Capture antibody conc: 1 μg/mL
Detection antibodies: NRP2 pAb Cat.#BAF2215, R&D Systems
   HRS C-terminal mAb Clone#13C8
   HRS N-terminal mAb Clone #12H6
Detection antibody conc: 0.5 μg/mL
Secondary reagent: Streptavidin SULFO-TAG, # R32AD-1, 500 μg/mL
Secondary reagent conc: 1 μg/mL
Substrate: MSD Read Buffer T (4×) with Surfactant #R92TC-1

Antibody Characterization Studies.

Surface plasmon resonance (SPR) methods were used to characterize the binding kinetics and affinities of the antibodies towards HRS proteins. SPR experiments were conducted on a Bio-Rad ProteOn XPR36 Protein Interaction Array instrument. HRS proteins were immobilized on different channels of a ProteOn GLC sensor chip through amine coupling. Each antibody at a series of different concentrations was flowed over the immobilized proteins. The sensor chip surface was regenerated between each antibody run to remove bound antibodies. The resulting sensograms were analyzed in the ProteOn Manager Software, and fitted globally to a bivalent analyte model to obtain on-rates (kₐ) and off-rates (k_{d}). The equilibrium dissociation constant (K_{D}) for each antibody-protein pair is the ratio of k_{d}/kₐ.
Running buffer: 1×PBS with 0.005% Tween-20
Amine coupling: ProteOn Amine Coupling Kit (Bio-Rad #1762410)
Ligand coupling buffer: Sodium acetate pH 5.5
Regeneration buffer: 10 mM HCl

Protein-Protein Interaction Studies.

Surface plasmon resonance (SPR) methods were used to demonstrate interactions between protein partners. SPR experiments were conducted on a Bio-Rad ProteOn XPR36 Protein Interaction Array instrument. Proteins were immobilized on different channels of ProteOn GLC sensor chips through amine coupling. Analyte proteins were flowed over the immobilized proteins. The sensor chip surface was regenerated between each analyte run to remove interacting proteins. Data was double referenced against an interspot (untreated chip surface) and a blank surface (activated and deactivated for amine coupling).
Running buffer: 50 mM HEPES, 300 mM NaCl, 5 mM CaCl₂, 0.005% Tween-20, pH 7.4
Amine coupling: ProteOn Amine Coupling Kit (Bio-Rad #1762410)
Ligand coupling buffer: Sodium acetate (pH 4.0, 4.5, 5.0, 5.5 depending on pI of protein)
Regeneration buffer: 10 mM glycine pH 2.0

Commercial proteins reagents (proteins are derived from human sequences unless noted):
NRP2-Fc (R&D Systems #2215-N2)
NRP1-His (R&D Systems #3870-N1)
Mouse PLXNA1-His (R&D Systems #4309-PA)
SEMA3C-Fc (R&D Systems #5570-S3)
Mouse SEMA3F-Fc (R&D Systems #3237-S3)
Mouse NRP2-Fc (R&D Systems #7988-N2)
Rat NRP2-Fc-His (R&D Systems #567-N2)
VEGF-C(R&D Systems #9199-VC/CF)
VEGF-A₁₆₅ (Peprotech #100-20)
VEGF-A₁₄₅ (R&D Systems #7626-VE-CF)
VEGF-A₁₂₁ (Peprotech #100-20A)
PlGF-2 (Peprotech #100-56)
Heparin (StemCell #07980)

Immunofluorescence Assays on Cultured Cells.

Reagents:
PE anti-human IgG Fc Antibody, clone HP6017, Biolegend cat 409304
Mouse Anti-HRS monoclonal antibody (1-96) Clone 1C8, Abnova cat H00003035-M01
Mouse IgG2a, Isotype control antibody (MOPC-173), Biolegend cat 400223
Recombinant Human VEGF-C Protein, R&D Systems, cat 2179-VC-025/CF
Formaldehyde, 16%, methanol free, Ultra Pure, Polysciences, cat 18814-10
Hoechst 33342, Trihydrochloride, Trihydrate, ThermoFisher Scientific, cat H1399
Fc-HRS (2-60) was prepared as described in PCT application PCT/US2014/029699
Gibco DMEM, High Glucose, ThermoFisher Scientific, cat 11965092
PolyJet™ In Vitro DNA Transfection Reagent, SignaGen, cat SL100688
Neuropilin 2 (NRP2) (NM_003872) Human ORF Clone, Origene, cat RG220706
Collagen Coating Solution, Cell Applications Cat 125-100
   1×PBS containing 1% BSA, 0.9 mM CaCl₂ and 20 mM glucose
   Binding buffer containing 1% normal mouse IgG (sigma cat 18765) and 2.5% (Human Fc Receptor Binding Inhibitor, ebioscience 14-9161-73).

Cell Culture and Transfection.

HEK293T cells were cultured in DMEM containing 10% FBS and 1% Penicillin/Streptomycin. Cells were seeded in 6-well plates the night before transfection. 1 g of plasmid DNA encoding a NRP2a-GFP fusion protein was pre-complexed with PolyJet transfection reagent according to the manufacturer's protocol and subsequently added to cells. Media was changed 16 hours-post transfection and transfected cells were passed to 96-well plates for staining.

Immunofluorescence Assays on Cultured Cells.

Binding and quantitation of Fc-HRS (2-60) to cell-expressed NRP2 was achieved using immunofluorescence microscopy. Fc-HRS (2-60) was pre-complexed for 1 hour at room temperature with PE-conjugated anti-Fc at a ratio of 2:1. HEK293T cells previously transfected with NRP2v2-GFP were passed the night before staining to 96 well Greiner Clear flat bottomed Microplates pre-coated with collagen coating solution. Supernatants were removed and cells were washed 1 time with binding buffer. Cells were then fixed with 50 μL of 3.7% methanol-free formaldehyde for 20 minutes at room temperature. Cells were washed 2× with binding buffer and then blocked with 100 μL of blocking buffer for 1 hour at room temperature. The cells were then washed one time with binding buffer and then incubated with 50 μL of staining complex overnight at 4 degrees Celsius. Cells were then washed 3 times with binding buffer, and then nuclei were counter stained with 2 g/mL Hoechst diluted in DPBS for 10 minutes at room temperature. The Hoechst stain was replaced with 1×PBS and subsequently read on an IN Cell Analyzer 2200. 20× images were acquired and analyzed using In Cell Analyzer 1000 Workstation software. Segmentation of the cell mask was achieved using the GFP channel, and the average PE signal intensity was determined within this mask above a threshold intensity of 5000 (termed GFP Bright cells).

Stable NRP2 expressing cell pool generation. A plasmid (Origene Technologies Cat#RC220706) encoding the NRP2 variant 2 transcript NM_003872 fused to a Myc-DDK tag was purchased. The vector was PCR amplified using Q5 polymerase (New England Biolabs Cat#M0491) with the following primer pairs:

(SEQ ID NO: 418)

5′-TGAGGATGACAAAGATTTGCAGCT-3′

(SEQ ID NO: 419)

5′-ACCGCGGCCGGCCGTTTATGCCTCGGAGCAGCACTT-3′

(SEQ ID NO: 420)

5′-AGTGCCAAGCAAGCAACTCAAA-3′

(SEQ ID NO: 421)

5′-AAGTGCTGCTCCGAGGCATAAACGGCCGGCCGCGGT-3′

The resulting PCR products were then fused, cut with MfeI/AgeI (New England Biolabs Cat#R3589, R3552), and ligated into a vector fragment of RC220706 cut with the same enzymes. This vector, containing an untagged NRP2v2 transcript, was then linearized and re-suspended in 10 mM Tris-0.1 mM EDTA. Suspension Expi293 cells (ThermoFisher Cat#A14527), were grown in expression medium (ThermoFisher Cat#A1435101) at 37° C. and 8% CO₂. The linearized plasmid described above was transfected into Expi293 cells using an SF Cell Line 4D-Nucleofector® X Kit L (Lonza Cat#V4XC-2012) and standard protocol T-030 for suspension HEK293 cells. Cells were allowed to recover in static culture for 17 hours, transferred to suspension and recovered an additional 72 hours, and then were selected with 200-350 μg/mL G418 in 50 μg increments (ThermoFisher Cat#10131035). Cell densities and viabilities were monitored for a period of 3 weeks, with fresh media/antibiotic replacement every 2-3 days. Upon recovery of viabilities to >95%, stably transfected cell pools were re-suspended in freezing media and archived.

Flow Cytometry-Based Assay for Fc-HRS (2-60) Binding to NRP2-Expressing Cells
Immobilized TCEP Disulfide Reducing Gel (Thermo Scientific #77712)
PBS/EDTA (0.5 M EDTA in PBS)
EZ-Link™ Maleimide-PEG11-Biotin (Thermo Scientific #21911)
Spin Columns (Thermo Scientific #69705)
Zeba™ Spin Desalting Columns, 40K MWCO (Thermo Scientific #87770)
Pierce Biotin Quantitation Kit (Thermo Scientific #28005)
Streptavidin-PE (Thermo Scientific #12-4317-87)
Anti-NRP2-APC clone 257103 (R&D Systems # FAB22151A)
Propidium iodide solution (Miltenyi Biotec #130-093-233)

Biotinylation of Fc-HRS (2-60).

Fc disulfide bonds in Fc-HRS (2-60) were reduced using TCEP gel equilibrated with PBS/EDTA, and the sample was separated using a spin column. Biotinylation was performed using maleimide-PEG11-biotin reagent with a 2 hour reaction at room temperature, and free reagent removed using a Zeba column. Degree of biotinylation was determined to be 3.35 biotins/molecule using the Pierce Biotin Quantitation kit according to the manufacturer's instructions.

Flow cytometry.

Biotinylated Fc-HRS (2-60) was incubated for 1 hour on ice with streptavidin-PE at a 1:2 molar ratio to form a staining complex. The staining complex was then added to stably expressing Expi293-NRP2 cells along with titrated anti-HRS antibodies and incubated 30-60 min on ice. Final concentrations were 43.75 nM (biotinylated Fc-HRS (2-60)) and 87.5 nM (streptavidin-PE). Cells were pelleted and washed as described above, and stained with anti-NRP2-APC (1:20) and resuspended in FWB buffer along with 1 μg/mL propidium iodide for viability gating. Samples were acquired on a Cytoflex S flow cytometer (Beckman Coulter), and the percentage of streptavidin-PE+/NRP2+ cells in the viable singlet gate was determined.

Statistical Analysis.

Data are expressed as mean±SEM or as individual data points, except where noted. In experiments in which animals were euthanized due to tumor burden or body weight loss, the terminal tumor volume was carried forward for statistical analysis. In cases where an animal was found dead but did not have a large tumor (cause of mortality unknown), the animal's data were removed prior to statistical analysis. Significance of difference over time was tested with a 2-way repeated measures ANOVA followed by Dunnett's post-hoc test. Group comparisons were conducted using 1-way ANOVA (parametric or Kruskal Wallis, as noted in the figure legends). A p value ≤0.05 was considered significant.

### Example 1

### Characterization of Antibodies to HRS (Histidyl tRNA Synthetase)

Antibodies to HRS were generated as described in the Materials and Methods and characterized by surface plasmon resonance (SPR) as described herein. The results are presented in Table E1, and demonstrate that antibodies ATYR13E9 and 13C8 demonstrate high affinity, sub nanomolar binding affinity to mouse HRS. Antibody clone ATYR13C8 also demonstrates good cross reactivity between mouse and human HRS, and specificity towards the C-terminal region of HRS. By contrast, antibody clone ATYR13E9 shows high specificity of mouse over human HRS and binds to the N-terminal region of HRS. The combination of antibodies therefore provides powerful tools to dissect the potential role of HRS in cancer progression, and immune modulation. Table E1 below summarizes certain binding characteristics of the antibodies described herein.

TABLE E1
ANTI-HARS (HRS) ANTIBODY CHARACTERISTICS

| | | | | | | Human | Cyno | Mouse | WHEP | | |
|---|---|---|---|---|---|---|---|---|---|---|---|
| | | | Immu- | | Species | HARS | HARS | HARS | domain | ΔWHEP | SV-11 |
| Clone | Species | Isotype | nogen | Epitope | React. | Kd (M) | Kd (M) | Kd (M) | Kd (M) | Kd (M) | Kd(M) |
| 12H6 | Mouse | IgG2b | 1-509 | 2-40; | Human | 3.3E−09 | ND | N/A | 4.1E−12 | N/A | 1.9E−09 |
| | | kappa | | | | | | | | | |
| 13E9 | Mouse | IgG1 | 1-60 | 2-45 | Mouse >>> | 1.7E−07 | ND | 6.8E−11 | 2.5E−07 | N/A | 8.0E−08 |
| | | kappa | mouse | | Human | | | | | | |
| | | | HARS | | | | | | | | |
| 13C8 | Mouse | IgG1 | 61-506 | 152-398 | Human & | 4.3E−10 | ND | 2.1E−10 | N/A | 1.9E−10 | N/A |
| | | kappa | | | Mouse | | | | | | |
| 8D10 | Mouse | IgG2b | 1-509 | 61-398 | Only | ND | ND | ND | ND | ND | ND |
| | | kappa | | | Human | | | | | | |
| | | | | | Tested | | | | | | |
| 11A7 | Mouse | IgG | 1-509 | 1-60 | Only | ND | ND | ND | ND | ND | ND |
| | | | | | Human | | | | | | |
| | | | | | Tested | | | | | | |
| KL31- | Human- | IgG1m | N/A | 1-60 | Human = | 7.6e−11 | 8.2e−11 | 6.14e−10 | ND | ND | ND |
| 478 | ized | (zf) | | | Cyno >> | | | | | | |
| | | kappa | | | Mouse | | | | | | |
| KL31- | Human- | IgG4 | N/A | 1-60 | Human = | 1.04e−10 | 1.08e−10 | 9.42e−10 | ND | ND | ND |
| 240 | ized | kappa | | | Cyno >> | | | | | | |
| | | | | | Mouse | | | | | | |
| KL31- | Human- | IgG1m | N/A | 1-60 | ND | 1.22e−08 | ND | ND | ND | ND | ND |
| 600 | ized | (zf) | | | | | | | | | |
| | | kappa | | | | | | | | | |
| KL31- | Human- | IgG1m | N/A | 1-60 | ND | 3.25e−08 | ND | ND | ND | ND | ND |
| 523 | ized | (zf) | | | | | | | | | |
| | | kappa | | | | | | | | | |
| KL31- | Human- | IgG1m | N/A | 1-60 | Human = | 1.40e−09 | 1.20e−09 | 5.10e−09 | ND | ND | ND |
| 241 | ized | (zf) | | | Cyno >> | | | | | | |
| | | kappa | | | Mouse | | | | | | |
| KL31- | Mouse | IgG1kappa | N/A | 1-60 | ND | 6.79e−09 | ND | ND | ND | ND | ND |
| 275 | | | | | | | | | | | |
| KL31- | Mouse | IgG2a | N/A | 1-60 | ND | 2.77e−07 | ND | ND | ND | ND | ND |
| 313 | | kappa | | | | | | | | | |
| KL31- | Human- | IgG1m | N/A | 1-60 | Human = | 2.10e−10 | 2.00e−10 | 9.60e−10 | ND | ND | ND |
| 467 | ized | (zf) | | | Cyno >> | | | | | | |
| | | kappa | | | Mouse | | | | | | |
| KL31- | Human- | IgG1m | N/A | 1-60 | ND | 6.70e−10 | ND | ND | ND | ND | ND |
| 261 | ized | (zf) | | | | | | | | | |
| | | kappa | | | | | | | | | |
| KL31- | Human- | IgG4-2 | N/A | 1-60 | Human = | 3.00e−10 | 2.90e−10 | 1.40e−09 | ND | ND | ND |
| 356 | ized | kappa | | | Cyno >> | | | | | | |
| | | | | | Mouse | | | | | | |
| KL31- | Human- | IgG1m | N/A | 1-60 | Human = | 6.40e−11 | 5.60e−11 | 3.70e−10 | ND | ND | ND |
| 532 | ized | (zf) | | | Cyno >> | | | | | | |
| | | kappa | | | Mouse | | | | | | |
| KL31- | Human- | IgG1m | N/A | 1-60 | ND | 2.50e−10 | ND | ND | ND | ND | ND |
| 131 | ized | (zf) | | | | | | | | | |
| | | kappa | | | | | | | | | |
| KL31- | Human- | IgG4-2 | N/A | 1-60 | Human = | 1.16e−10 | 1.69e−10 | 1.25e−09 | ND | ND | ND |
| 513 | ized | kappa | | | Cyno >> | | | | | | |
| | | | | | Mouse | | | | | | |
| KL31- | Human- | IgG1m | N/A | 1-60 | ND | ND | ND | ND | ND | ND | ND |
| 254 | ized | (zf) | | | | | | | | | |
| | | kappa | | | | | | | | | |
| KL31- | Human- | IgG1m | N/A | 1-60 | ND | ND | ND | ND | ND | ND | ND |
| 515 | ized | (zf) | | | | | | | | | |
| | | kappa | | | | | | | | | |
| KL31- | Human- | IgG1m | N/A | 1-60 | ND | ND | ND | ND | ND | ND | ND |
| 135 | ized | (zf) | | | | | | | | | |
| | | kappa | | | | | | | | | |
| KL31- | Human- | IgG1m | N/A | 1-60 | ND | ND | ND | ND | ND | ND | ND |
| 470 | ized | (zf) | | | | | | | | | |
| | | kappa | | | | | | | | | |
| KL31- | Human- | IgG1m | N/A | 1-60 | ND | ND | ND | ND | ND | ND | ND |
| 316 | ized | (zf) | | | | | | | | | |
| | | kappa | | | | | | | | | |
| KL31- | Human- | IgG1m | N/A | 1-60 | ND | ND | ND | ND | ND | ND | ND |
| 567 | ized | (zf) | | | | | | | | | |
| | | kappa | | | | | | | | | |
| KL31- | Human- | IgG1m | N/A | 1-60 | ND | ND | ND | ND | ND | ND | ND |
| 481 | ized | (zf) | | | | | | | | | |
| | | kappa | | | | | | | | | |
| KL31- | Mouse | IgG1 | N/A | 1-60 | ND | ND | ND | ND | ND | ND | ND |
| 366 | | kappa | | | | | | | | | |
| | | D265A | | | | | | | | | |
| KL31- | Human- | IgG4-2 | N/A | 1-60 | ND | ND | ND | ND | ND | ND | ND |
| 449 | ized | kappa | | | | | | | | | |
| KL31- | Human- | IgG1m | N/A | 1-60 | ND | ND | ND | ND | ND | ND | ND |
| 468 | ized | (za) | | | | | | | | | |
| | | kappa | | | | | | | | | |
| KL31- | Human- | IgG1m | N/A | 1-60 | ND | ND | ND | ND | ND | ND | ND |
| 418 | ized | (za) | | | | | | | | | |
| | | kappa | | | | | | | | | |
| AB04- | Human | IgG1m | N/A | 1-60 | Human > | 1.00e−11 | 6.2e−11 | 1.27e−10 | ND | ND | ND |
| 425 | | (zf) | | | Cyno > | | | | | | |
| | | kappa | | | Mouse | | | | | | |
| AB04- | Human | IgG4-2 | N/A | 1-60 | Human > | 1.5e−11 | 5.7e−11 | 1.45e−10 | ND | ND | ND |
| 168 | | kappa | | | Cyno > | | | | | | |
| | | | | | Mouse | | | | | | |
| AB04- | Human | IgG1m | N/A | 1-60 | ND | 9.00e−10 | ND | ND | ND | ND | ND |
| 121 | | (zf) | | | | | | | | | |
| | | kappa | | | | | | | | | |
| AB04- | Human | IgG1m | N/A | 1-60 | ND | 1.70e−07 | ND | ND | ND | ND | ND |
| 174 | | (zf) | | | | | | | | | |
| | | kappa | | | | | | | | | |
| AB04- | Human | IgG1m | N/A | 1-60 | ND | 1.90e−09 | ND | ND | ND | ND | ND |
| 411 | | (zf) | | | | | | | | | |
| | | kappa | | | | | | | | | |
| AB04- | Human | IgG1m | N/A | 1-60 | ND | 2.60e−10 | ND | ND | ND | ND | ND |
| 482 | | (zf) | | | | | | | | | |
| | | kappa | | | | | | | | | |
| AB04- | Human | IgG1m | N/A | 1-60 | ND | 4.60e−10 | ND | ND | ND | ND | ND |
| 276 | | (zf) | | | | | | | | | |
| | | kappa | | | | | | | | | |
| AB04- | Human | IgG1m | N/A | 1-60 | ND | 2.65e−10 | ND | ND | ND | ND | ND |
| 483 | | (zf) | | | | | | | | | |
| | | kappa | | | | | | | | | |
| AB04- | Human | IgG1m | N/A | 1-60 | ND | 1.85e−10 | ND | ND | ND | ND | ND |
| 365 | | (zf) | | | | | | | | | |
| | | kappa | | | | | | | | | |
| AB04- | Human | IgG1m | N/A | 1-60 | ND | 2.50e−11 | ND | ND | ND | ND | ND |
| 151 | | (zf) | | | | | | | | | |
| | | kappa | | | | | | | | | |
| AB04- | Human | IgG1m | N/A | 1-60 | ND | 6.00e−11 | ND | ND | ND | ND | ND |
| 160 | | (zf) | | | | | | | | | |
| | | kappa | | | | | | | | | |
| AB04- | Human | IgG1m | N/A | 1-60 | Human > | 4.80e−11 | 7.50e−11 | 1.30e−10 | ND | ND | ND |
| 439 | | (zf) | | | Cyno > | | | | | | |
| | | kappa | | | Mouse | | | | | | |
| AB04- | Human | IgG1m | N/A | 1-60 | ND | 2.60e−11 | ND | ND | ND | ND | ND |
| 380 | | (zf) | | | | | | | | | |
| | | kappa | | | | | | | | | |
| AB13- | Human | IgG4 | N/A | 1-60 | Human = | <1.0e−12 | <1.0e−12 | 2.5e−12 | ND | ND | ND |
| 112 | | kappa | | | Cyno > | | | | | | |
| | | | | | Mouse | | | | | | |
| AB13- | Human | IgG1m | N/A | 1-60 | Human = | <1.0e−12 | <1.0e−12 | <1.0e−12 | ND | ND | ND |
| 112 | | (za) | | | Cyno > | | | | | | |
| | | kappa | | | Mouse | | | | | | |
| AB13- | Human | IgG1m | N/A | 1-60 | Human = | 2.10e−10 | 2.40e−10 | 2.10e−10 | ND | ND | ND |
| 433 | | (zf) | | | Cyno > | | | | | | |
| | | kappa | | | Mouse | | | | | | |
| AB13- | Human | IgG1m | N/A | 1-60 | ND | 2.40e−11 | ND | ND | ND | ND | ND |
| 147 | | (zf) | | | | | | | | | |
| | | kappa | | | | | | | | | |
| AB13- | Human | IgG1m | N/A | 1-60 | ND | <5.0e−11 | ND | ND | ND | ND | ND |
| 227 | | (zf) | | | | | | | | | |
| | | kappa | | | | | | | | | |
| AB13- | Human | IgG1m | N/A | 1-60 | ND | <1e−12 | ND | ND | ND | ND | ND |
| 166 | | (zf) | | | | | | | | | |
| | | kappa | | | | | | | | | |
| AB13- | Human | IgG1m | N/A | 1-60 | Human = | <1e−12 | 2.00e−12 | <1e−12 | ND | ND | ND |
| 288 | | (zf) | | | Cyno > | | | | | | |
| | | kappa | | | Mouse | | | | | | |
| AB13- | Human | IgG4-2 | N/A | 1-60 | Human > | <1e−12 | 2.80e−11 | 2.30e−11 | ND | ND | ND |
| 288 | | kappa | | | Cyno > | | | | | | |
| | | | | | Mouse | | | | | | |
| AB13- | Human | IgG1m | N/A | 1-60 | ND | 1.50e−11 | ND | ND | ND | ND | ND |
| 259 | | (zf) | | | | | | | | | |
| | | kappa | | | | | | | | | |
| AB13- | Human | IgG4-2 | N/A | 1-60 | ND | 2.90e−11 | ND | ND | ND | ND | ND |
| 259 | | kappa | | | | | | | | | |
| AB13- | Human | IgG1m | N/A | 1-60 | ND | ND | ND | ND | ND | ND | ND |
| 181 | | (zf) | | | | | | | | | |
| | | kappa | | | | | | | | | |
| AB13- | Human | IgG1m | N/A | 1-60 | ND | ND | ND | ND | ND | ND | ND |
| 270 | | (zf) | | | | | | | | | |
| | | kappa | | | | | | | | | |
| AB13- | Human | IgG1m | N/A | 1-60 | ND | ND | ND | ND | ND | ND | ND |
| 166 | | (zf) | | | | | | | | | |
| | | kappa | | | | | | | | | |
| AB13- | Human | IgG1m | N/A | 1-60 | ND | ND | ND | ND | ND | ND | ND |
| 459 | | (zf) | | | | | | | | | |
| | | kappa | | | | | | | | | |

### Example 2

### Evaluation of Anti-Tumor Activity of Test Compounds on B16-F10 Mouse Melanoma in C57BL/6 Mice

This study was designed to investigate the in vivo anticancer potential of test compounds in a syngeneic mouse model using B16-F10 cells (a melanoma cancer model) prepared as described in Materials and Methods. The test antibody dosing regimen was initiated one day before cell injection and animal weights and tumor measurements were recorded three times a week until study termination.

Treatment Regimen.

Thirty (30) C57BL/6 mice (Envigo, female, 5-6 wks old) were used in this study. The animals were assigned to 3 study groups of 10 mice randomly, and housed as described in the Materials and Methods. The antibody dosing regimen is shown in Table E2 below; in brief animals received intraperitoneal injections of 200 μg/mouse of each of the control IgG antibodies, positive control antibodies (e.g., α-CTLA-4, α-PD-L1), and anti-HRS antibodies ATYR13C8 and ATYR13E9 (as described in the Materials and Methods), administered according to the protocol below (Table E2); starting one day before cancer cell implantation and then on day 6 and 13 post-cell implantation of cancer cells).

TABLE E2
GROUP TREATMENTS

| | | | | Route of | |
|---|---|---|---|---|---|
| | | | Dose | Administration | |
| Group | #Mice | Test Materials | (μg/mouse) | (ROA) | Frequency |
| 1 | 10 | Control IgG | 200 | IP | days −1, 6, 13 |
| 2 | 10 | αCTLA-4 + αPD-L1 | 200 + 200 | IP | days −1, 6, 13 |
| 3 | 10 | ATYR13C8 + ATYR13E9 | 200 + 200 | IP | days −1, 6, 13 |

* IP = Intraperitoneal injection

Summary of Results.

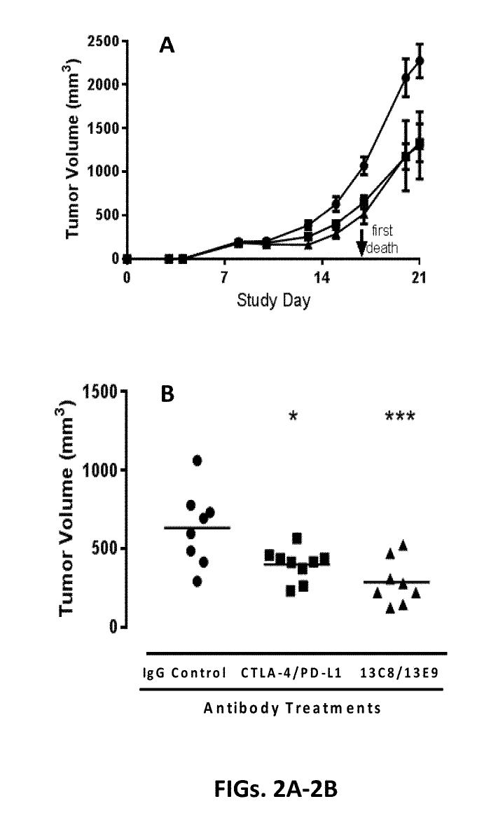

Animals bearing B16-F10 tumors and treated with 3 doses of ATYR13C8 plus ATYR13E9 showed a ˜40% reduced tumor volume, reaching statistical significance (p≤0.05) on Day 13 (FIG. 2A). Surprisingly the combination of an N-terminally directed anti-HRS antibody (clone ATYR13E9) and C-terminally directed antibody to HRS (clone ATYR13C8) inhibited B16-F10 Melanoma growth at least as well as the combination of bench mark anti-PD1 and anti-CTLA-4 antibodies at days 13, 15 and 17 (FIG. 2B shows day 15 data). There was no evidence of toxicity from animal body weight measurements and observations during the study (data not shown). These results demonstrate that anti-HRS antibodies have clear activity with a prophylactic and potentially therapeutic impact on cancer growth in this model system.

### Example 3

### Evaluation of Anti-Tumor Activity of Test Compounds on the Seeding to the Lung of Intravenously Injected B16-F10 Mouse Melanoma Cells in C57BL/6 Mice

This study aims to investigate the in vivo anticancer potential of test compounds in a syngeneic mouse model using B16-F10 cells to evaluate their potential impact on seeding of intravenously injected tumor cells to the lung, as determined via measuring tumor node number. The dosing regimen was initiated one day before cancer cell injection, animal weights were recorded three times a week. After 18 days the animals were sacrificed and the number of tumor nodes present on the lungs of each animal was recorded.

Treatment Regimen.

Thirty (30) C57BL/6 mice (Envigo, female, 5-6 wks old) were used in this study. The animals were assigned to 3 study groups of 10 mice randomly. The B16-F10 cell line was prepared for injection as described in the Materials and Methods. Note that in contrast to other examples, the cells were administered intravenously. Administration via this route creates the potential for tumor cells to lodge in the lungs and grow. The dosing regimen is shown in Table E3 below; In brief animals received injections of 200 μg/mouse of each control IgG, positive control antibodies, and anti-HRS antibodies (as described in the Materials and Methods), which were administered to mice intraperitoneally according to the protocol below (Table E3) starting one day before cancer cell implantation and then on day 6 and 13 post cancer implantation).

TABLE E3
GROUP TREATMENTS

| | | | Dose | | |
|---|---|---|---|---|---|
| Group | #Mice | Materials | (μg/mouse) | ROA | Frequency |
| 1 | 10 | Control IgG | 200 | IP | days −1, 6, 13 |
| 2 | 10 | αCTLA-4 + αPD-L1 | 200 + 200 | IP | days −1, 6, 13 |
| 3 | 10 | ATYR13C8 + ATYR13E9 | 200 + 200 | IP | days −1, 6, 13 |

* IP = Intraperitoneal injection

After 18 days, the number of tumor nodes, which are black due to production of melanin, were counted in the lungs.

Summary of Results.

There were no significant differences in body weight between group 1 (control IgG) and group 2 or 3 during treatments (data not shown), suggesting that both positive control antibodies and test antibodies have no toxicity in terms of statistically significant impacts on body weight loss.

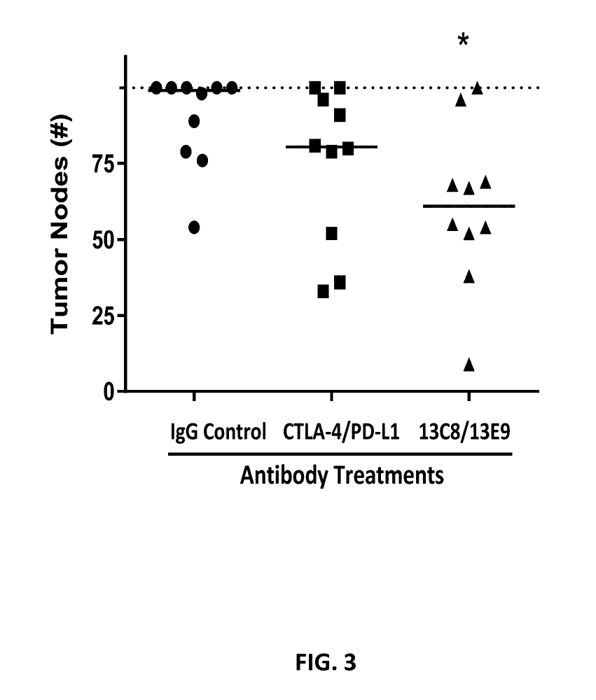

Five (5) mice in group 1 (control IgG), 2 mice in group 2 (anti-CTLA-4 and anti-PD-L1 positive control antibodies), and 1 mouse in group 3 (ATYR13C8 and ATYR13E9 anti-HRS antibodies) developed entirely black lungs, consistent with them becoming saturated with metastatic tumor nodules (FIG. 3). Lungs that were saturated with tumor nodules were assigned a value of 100 (just above the highest count recorded of 98 nodules). Because the data were not normally distributed, statistical comparisons were conducted using a Kruskal-Wallis non-parametric 1-way ANOVA. There were significantly reduced numbers of tumor nodules in group 3 (ATYR13C8 and ATYR13E9 anti-HRS antibodies) compared to group 1 (control IgG), but no significant reduction in group 2 (anti-CTLA-4 and anti-PD-L1).

These results demonstrate that anti-HRS antibodies, either alone or in combination with other anti-cancer therapeutics, have clear activity with a prophylactic and potentially therapeutic impact on preventing tumor growth in this model system.

### Example 4

### HRS Polypeptide Levels in Naïve C57/Bl6 Mice Compared to Mice into which B16-F10 Melanoma Cells have been Introduced, and the Impact of Various Treatments on HRS Polypeptide Levels

Terminal serum samples from the mice in studies from Examples 1 and 2 were analyzed to determine whether the tumors impacted circulating HRS polypeptide levels, and to establish whether the anti-HRS antibodies were effective in suppressing free HRS levels using the dosing regimens implemented. HRS levels were determined using N-terminal and full length specific ELISA assays (as described in the Materials and Methods) capable of detecting free mouse and human HRS.

Summary of Results.

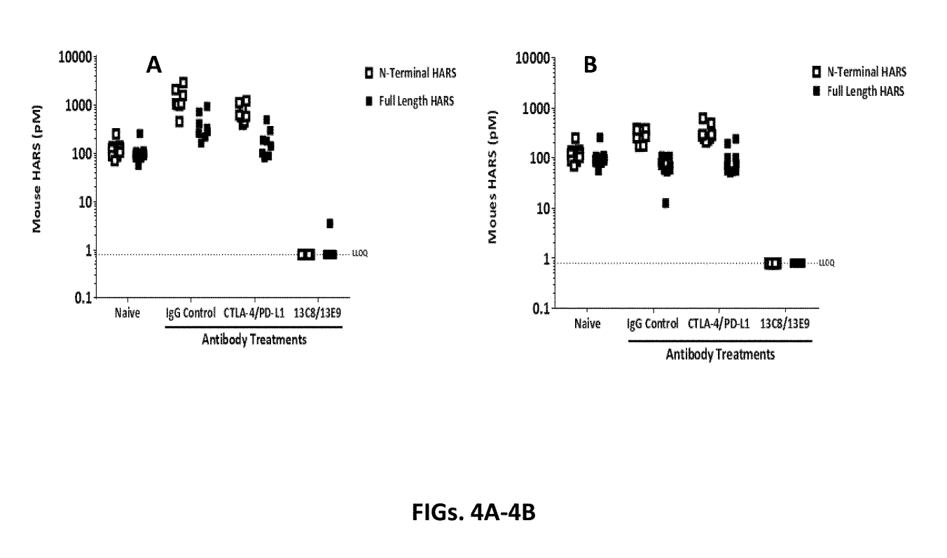

The analysis of HRS levels demonstrated that HRS levels were elevated in B16-F10 implanted mice treated with the IgG control antibody or the combination of anti-PD1 plus anti-CTLA-4 antibodies, in the studies in both Examples 1 and 2. (FIGS. 4A-4B). Circulating HRS levels were significantly higher in Example 1, in these groups, compared to the same groups in Example 2. This may be reflective of a higher tumor burden in Example 1 (subcutaneous tumor), compared to Example 2 (tumor nodules in the lungs), in these groups. In both examples, treatment with anti-HRS antibodies resulted in undetectable circulating levels of free HRS not bound to antibody.

These results confirm that the dosing regimens used are sufficient to result in undetectable free HRS under these experimental conditions, and suggest that tumor burden impacts HARS levels in circulation.

### Example 5

### Evaluation of the PK Characteristics of the ATYR13E9 and ATYR13C8 Anti-Hrs Antibodies Administered IV or IP in C57/Bl6 Mice

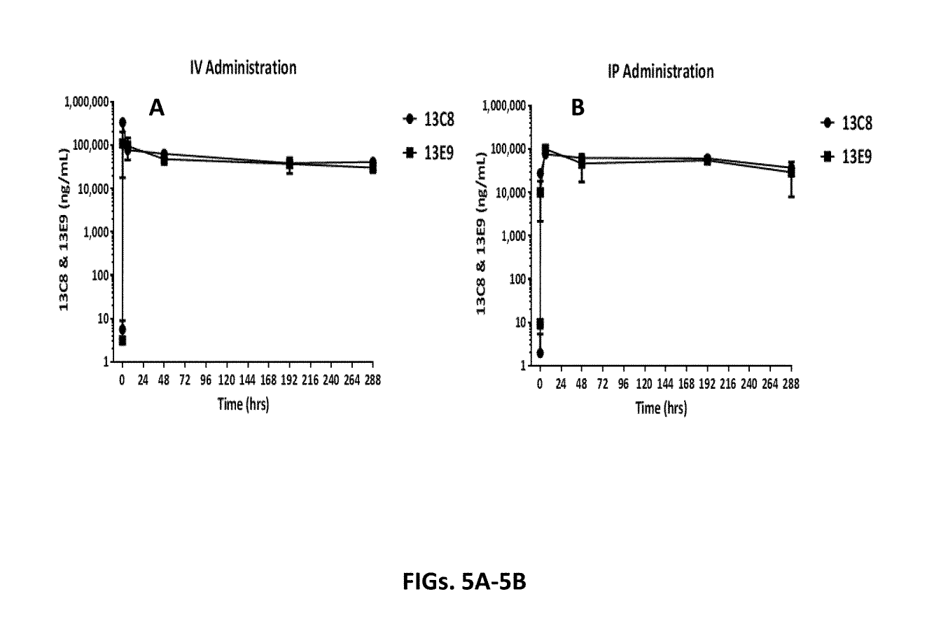

To evaluate the PK characteristics of anti-HRS antibodies ATYR13C8 and ATYR13E9, each antibody was administered IV or IP to normal C57/Bl6 mice at a dose of 6.6 mg/kg. Serum samples were taken at the time points indicated on FIGS. 5A-5B, and antibody levels determined using an ELISA assay as described in the Materials and Methods.

Summary of Results.

The results, shown in FIGS. 5A-5B, and summarized below in Table E4, demonstrate that both antibodies have similar clearance and half-lives after being administered IV and IP.

TABLE E4
SUMMARY OF PK CHARACTERISTICS OF
ANTIBODIES ATYR13C8 and ATYR13E9

| | | |
|---|---|---|
| Route of | ATYR13C8 | ATYR13E9 |

| | | | | |
|---|---|---|---|---|
| administration | IV | IP | IV | IP |

| | |
|---|---|
| Dose (mg/kg) | 6.6 |

| | | | | |
|---|---|---|---|---|
| Cmax (μg/mL) | 337 | 76 | 110 | 100 |
| Half Life (hr) | 333 | 293 | 368 | 325 |
| Vz (mL/kg) | 96 | 84 | 120 | 106 |
| Cl (mL/hr/kg) | 0.20 | 0.20 | 0.23 | 0.23 |
| BA % | | 100 | | 100 |

### Example 6

### A Single Anti-HRS Antibody (ATYR13E9) Targeting the N-Terminal Domain of HRS is Significantly Effective in Reducing Tumor Growth

This study aims to investigate the in vivo anticancer potential of test compounds in a syngeneic mouse model using B16-F10 cells (a melanoma cancer model) in C57BL/6 mice. In this study the anti-HRS antibodies ATYR13E9 and ATYR13C8 were also administered separately.

Treatment Regimen.

Forty-eight (48) C57BL/6 mice (Envigo, female, 7-8 wks old) were used in this study. The animals were assigned to 6 study groups of 8 mice randomly. The B16-F10 cell line was prepared as described in the Materials and Methods. The dosing regimen is shown in Table E5 below; In brief animals received injections of 200 μg/mouse of each control IgG, positive control antibodies (αCTLA-4 & αPD-L1), and anti-HRS antibodies (as described in the Materials and Methods), which was administered to mice intraperitoneally according to the protocol below (Table E5) starting one day before cancer cell implantation and then on day 6, 13 and 17 post implantation).

TABLE E5
GROUP TREATMENTS

| | | | Dose | | |
|---|---|---|---|---|---|
| Group | #Mice | Materials | (μg/mouse) | ROA | Frequency |
| 1 | 8 | Control no tumor no | 0 | NA | no treatment |
| | | treatment | | | |
| 2 | 8 | Control IgG1 | 400 | IP | days −1, 6, 13, 17 |
| 3 | 8 | αCTLA-4 + αPD-L1 | 200/200 | IP | days −1, 6, 13, 17 |
| 4 | 8 | ATYR13C8 + ATYR 13E9 | 200/200 | IP | days −1, 6, 13, 17 |
| 5 | 8 | ATYR13C8 + IgG1 (control) | 200/200 | IP | days −1, 6, 13, 17 |
| 6 | 8 | ATYR 13E9 + IgG1 (control) | 200/200 | IP | days −1, 6, 13, 17 |

Summary of Results.

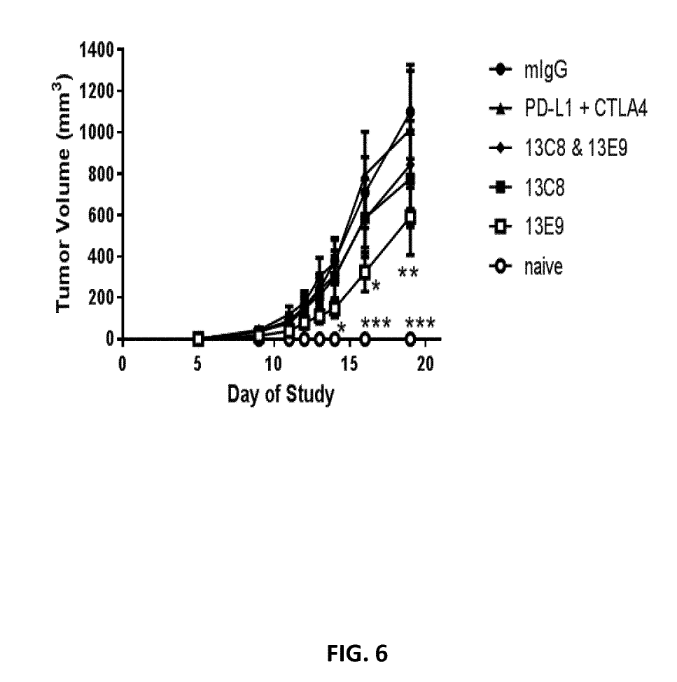

Animals bearing B16-F10 tumors treated with 4 doses of ATYR13C8 plus ATYR13E9 together, as well as each antibody separately showed reduced mean tumor volume, compared to IgG1 control and anti-PD-L1 plus anti-CTLA-4 control groups (FIG. 6). Surprisingly, the N-terminally directed anti-HRS antibody (clone ATYR13E9) alone inhibited B16-F10 melanoma growth effectively (FIG. 6). There was no evidence of toxicity from body weight measurements and observations during the study (data not shown).

These results demonstrate that a single anti-HRS antibody has clear activity with a prophylactic and potentially therapeutic impact on cancer growth in this model system.

### Example 7

### Anti-HRS Antibodies in Combination with PD-1 Pathway Blockade Cause the Regression of 4T1 Tumors in a Mouse Syngeneic Mouse Model, and Elicit a Memory Response Conferring Resistance to Tumor Cell Re-Inoculation

This study aims to investigate the in vivo anticancer potential of test compounds in a syngeneic mouse model using 4T1 cells (a model of triple negative breast cancer). Cells were prepared as described in Materials and Methods. In this study the anti-HRS antibody ATYR13E9 was administered separately and in combination with anti-PD-1 and anti-PD-L1 antibodies to assess whether there was an effect on 4T1 tumor growth by blocking HRS alone or in combination with anti-PD-1 and anti-PD-L1 antibodies. The dosing regimen was initiated one day before cell injection. The animal weights and tumor measurements were recorded three times a week by a person blinded to the treatments.

Treatment Regimen.

Seventy (70) Balb/c mice (Envigo, female, 5-6 wks old) were used in this study. The animals were assigned to 7 study groups of 10 mice randomly. The 4T1 cell line was expanded for injection as described in the Materials and Methods. The dosing regimen is shown in Table E6 below; In brief animals received injections of 200 μg/mouse of each control IgG, positive control antibodies, and anti-HRS antibodies (as described in the Materials and Methods), which were administered according to the protocol below (Table E6) to mice intraperitoneally, starting one day before cancer cell implantation, and then on day 6 and 13 post implantation). On Day 67 post initial (Day 0) 4T1 tumor inoculation, surviving animals were re-inoculated with 4T1 cells on the right flank, including the animals that had previously been naïve of both tumor and treatment. Monitoring of tumor volume and body weight continued until Day 99.

TABLE E6
GROUP TREATMENTS

| | | | Dose | | |
|---|---|---|---|---|---|
| Group | N | Material | (μg/mouse) | ROA | Frequency |
| 1 | 10 | αPD-1 + | 200 | IP | days −1, 6, 13 |
| | | IgG (control) | 200 | IP | days −1, 6, 13 |
| 2 | 10 | ATYR13E9 + | 200 | IP | days −1, 6, 13 |
| | | αPD-L1 (control) | 200 | IP | days −1, 6, 13 |
| 3 | 10 | αPD-L1 + | 200 | IP | days −1, 6, 13 |
| | | IgG (control) | 200 | IP | days −1, 6, 13 |
| 4 | 10 | ATYR13E9 + | 200 | IP | days −1, 6, 13 |
| | | IgG (control) | 200 | IP | days −1, 6, 13 |
| 5 | 10 | IgG isotype | 400 | IP | days −1, 6, 13 |
| | | control (control) | | IP | |
| 6 | 10 | ATYR13E9 + | 200 | IP | days −1, 6, 13 |
| | | αPD-1 | 200 | IP | days −1, 6, 13 |

| | | |
|---|---|---|
| 7 | 10 | N/A: no treatment, no tumor |

Summary of Results.

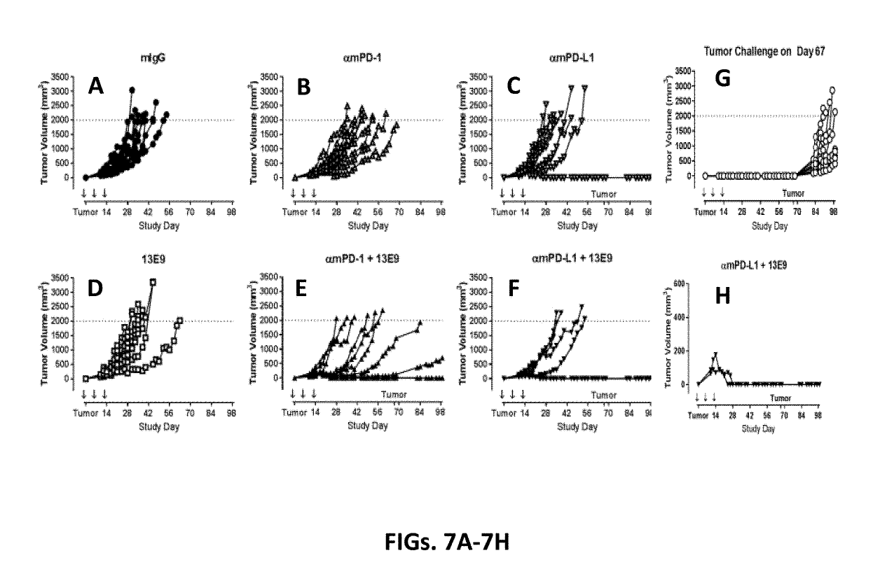

(FIG. 7) Animals bearing 4T1 tumor cells treated with 3 doses of anti-HRS antibody ATYR13E9 alone or together with either an anti-PD1 or anti-PD-L1 antibody showed more effective inhibition of tumor growth compared to control IgG beginning at study day 33. (FIGS. 7D-7F). Surprisingly both the combination of the anti-HRS antibody (clone ATYR13E9) in combination with an anti-PD-L1 antibody (FIG. 7F) and with an anti-PD-1 antibody were effective (FIG. 7E).

Animals inoculated with 4T1 tumor cells on Day 67 included 10 previously naïve mice (group 7 in Table E6) and 4 animals that had had treatment-associated tumor regression. These included 1 animal that had received anti-PD-1 antibodies (Group 1), 1 animal that had received ATYR13E9 plus anti-PD-1 antibodies and 2 animals that had been received ATYR13E9 and anti-PD-L1 antibodies. Surprisingly, while all of the previously naïve animals grew tumors, animals in which tumors had previously regressed did not grow new tumors. These data potentially suggest that anti-HRS antibodies contribute to the development of long lasting immune suppression of both cancer initiation and growth, including for example, the inhibition of metastatic tumor growth, initiation, or re-initiation, after treatment

There was no evidence of toxicity from body weight measurements and observations during the study (data not shown).

These results demonstrate that an anti-HRS antibody, in combination with either an anti-PD-1 or anti-PD-L1 antibody provides for a surprisingly more effective anti-tumor combination for 4T1 tumor cells. These successful treatments conferred resistance to the same tumor cell type administered ˜7.5 weeks after most recent antibodies administration, suggesting a memory response to the tumor had been successfully conferred.

These results demonstrate that an anti-HRS antibody, for example, in combination with either an anti-PD-1 or an anti-PD-L1 antibody, has clear potential for a prophylactic and therapeutic impact on cancer growth in this model of breast cancer, as well as the potential to result in effective combinations with other anti-cancer therapeutics, including for breast and other cancers. The development of long lasting immune suppression also suggests a role for the anti-HRS antibodies in preventing the recurrence of cancer, and in suppressing the development of metastatic cancer.

### Example 8

### Human Tumors Secrete HRS after Implantation into an Immunocompromised Mouse Model

This study aims to investigate whether human cancer cells secrete HRS when implanted into immunocompromised mice. In this study human A549 cells prepared as described in Materials and Methods were inoculated at two different cell numbers as described in Table E7. Human HRS was measured in terminal serum samples using an ELISA specific for the human protein as described in the Material and Methods. The animal weights and tumor measurements were recorded three times a week

Treatment Regimen.

Thirty-two (32) athymic nude mice (Foxn1^{nu/nu} JAX #002019, female, 5-6 wks old) were used in this study. Nude mice lack T cell development and are unable to mount a robust cell-mediated immune response, permitting growth of human cancer cell lines. The animals were assigned to 4 study groups of 8 mice randomly. The human A549 cell line was expanded for injection as described in the Materials and Methods and implanted on Day 0 according to the groups below (Table E7). Negative controls included a no cell/no treatment group (naïve) and a group that received the vehicle used with cell implantation (matrigel). Termination, including serum collection, was conducted on Day 42.

TABLE E7
GROUP TREATMENTS

| Group | #Mice | Materials | ROA | Frequency |
|---|---|---|---|---|
| 1 | 8 | A549 @ 1 × 10⁷ | SC | Once (Day 0) |
| 2 | 8 | A549 @ 2 × 10⁶ | SC | Once (Day 0) |
| 3 | 8 | Matrigel | SC | Once (Day 0) |
| 4 | 8 | Naive | NA | |

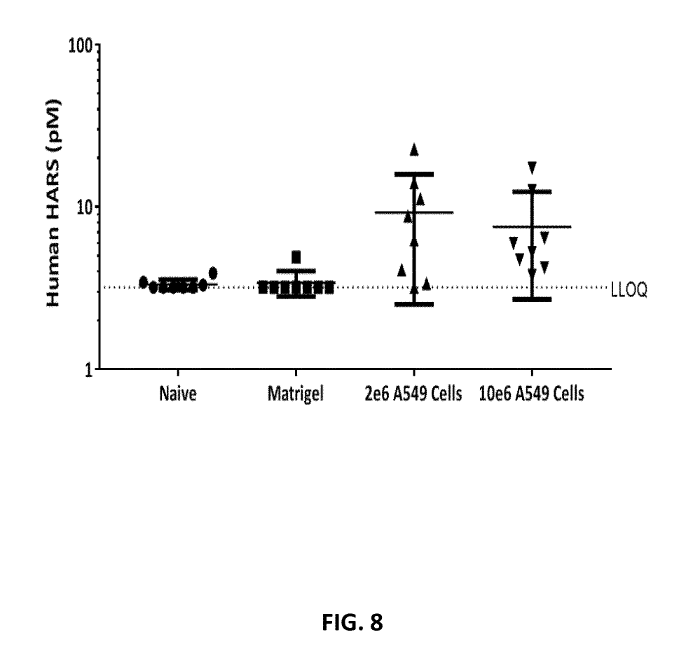

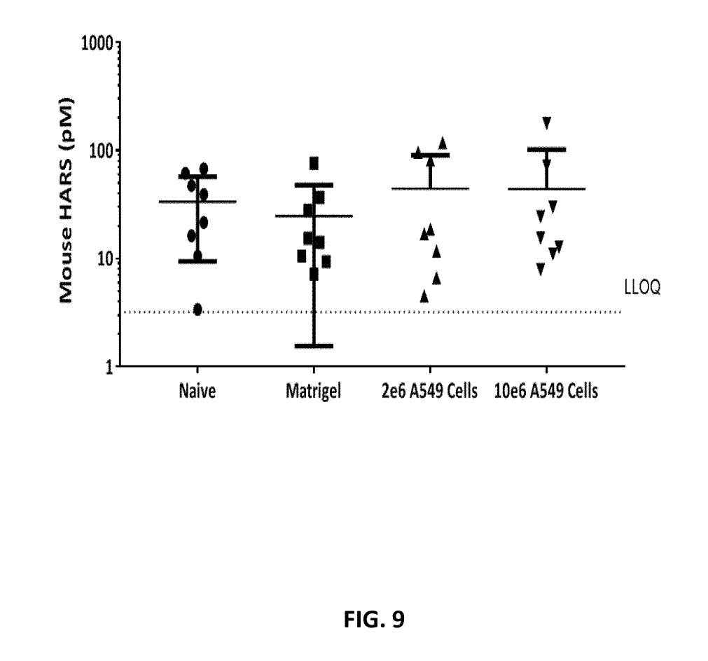

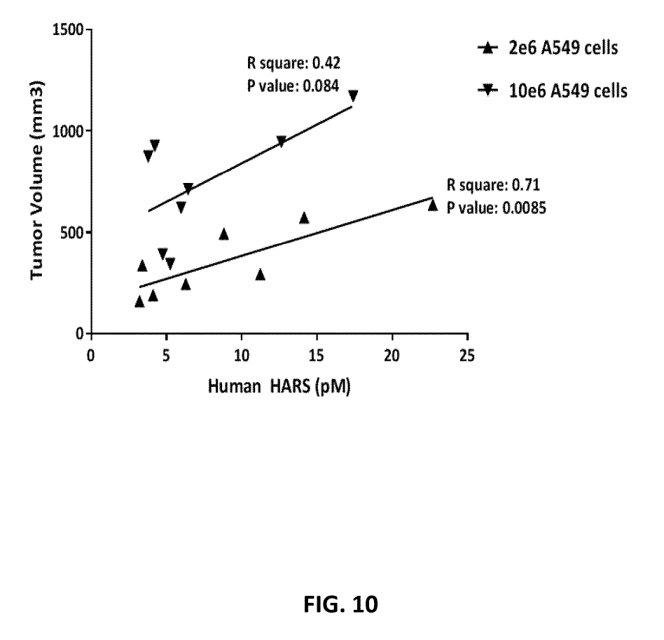

All animals in groups 1 and 2 implanted with human A549 cells grew tumors. At termination on Day 42, tumors ranged from 343-1170 mm³ in group 1 and from 162-636 mm³ in group 2, with significantly larger tumor size associated with higher cell number inoculated in group 1 (see FIG. 10). As expected, no tumors were observed or palpated in groups 3 and 4. Using ELISAs specific to human HRS as described in Materials and Methods, human HRS (FIG. 8) was surprisingly detectable in the serum of animals bearing a human A549 tumor, but not in human cell negative controls. Mouse HRS was readily detected in serum from all groups and not significantly different in animals bearing human A549 xenografts vs human cell negative controls (FIG. 9). Linear regression analysis performed on data obtained in tumor bearing animals shows a positive correlation between Human HRS and tumor size, suggesting a surprisingly tight relationship between human HRS and human tumor growth (FIG. 10).

### Example 9

### Combination of PD-1 Pathway Blockade and Anti-HRS Antibodies Inhibit Tumor Growth in the CT26 Tumor Model

This study aims to investigate the in vivo anti-cancer potential of an anti-HRS antibody alone or in combination with either anti-PD-1 or anti-PD-L1 antibodies in a syngeneic mouse model using CT26 cells (a model of colon cancer) prepared as described in Materials and Methods. In this study the dosing regimen (Table E8) was initiated one day before cell injection. The animal weights and tumor measurements were recorded three times a week.

Treatment Regimen.

Seventy (70) Balb/c mice (Envigo, female, 5-6 wks old) were used in this study. The animals were assigned to 7 study groups of 10 mice randomly. The CT26 cell line was expanded for injection as described in the Materials and Methods. The dosing regimen is shown in Table E8 below; In brief animals received injections of 200 μg/mouse of each control IgG, positive control antibodies, and anti-HRS antibodies as described in the Materials and Methods, which was administered to mice intraperitoneally starting one day before cancer cell implantation and on days 4, 7, 10, 14 and 17 post implantation) according to the protocol below (Table E8).

TABLE E8
GROUP TREATMENTS

| | | | Dose | | |
|---|---|---|---|---|---|
| | | | (μg/ | | |
| Group | #Mice | Materials | mouse) | ROA | Frequency |
| 1 | 10 | Naïve; | NA | NA | NA |
| | | no tumor, | | | |
| | | no treatment | | | |
| 2 | 10 | IgG control | 400 | IP | Day −1, 4, 7, 10, |
| | | antibody | | | 14, 17 |
| 3 | 10 | αPD-1 + | 200 | IP | Day −1, 4, 7, 10, |
| | | IgG (control) | 200 | | 14, 17 |
| 4 | 10 | αPD-L1 + | 200 | IP | Day −1, 4, 7, 10, |
| | | IgG (control) | 200 | | 14, 17 |
| 5 | 10 | ATYR13E9 + | 200 | IP | Day −1, 4, 7, 10, |
| | | IgG (control) | 200 | | 14, 17 |
| 6 | 10 | ATYR13E9 + | 200 | IP | Day −1, 4, 7, 10, |
| | | αPD-L1 | 200 | | 14, 17 |
| 7 | 10 | ATYR13E9 + | 200 | IP | Day −1, 4, 7, 10, |
| | | αPD-1 | 200 | | 14, 17 |

Summary of Results.

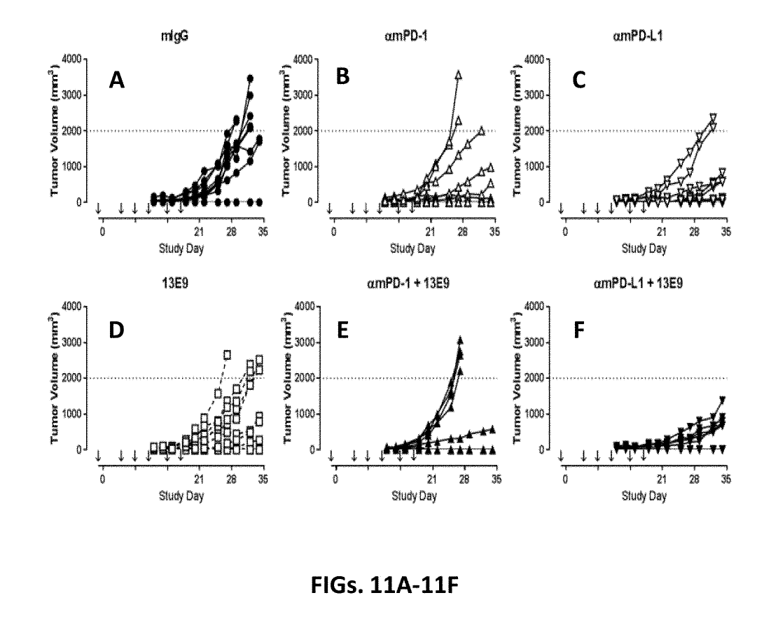

There was no evidence of toxicity from weight measurements and observations during the study (data not shown). Animals bearing CT26 tumors treated with 6 doses of ATYR13E9 alone and together with an anti-PD-L1 antibody showed more effective inhibition of tumor growth compared to control IgG beginning at study day 27 (FIGS. 11A-11F). Surprisingly both the combination of the anti-HRS antibody (clone ATYR13E9) in combination with an anti-PD-L1 antibody and with an anti-PD-1 antibody were both effective.

These results demonstrate that an anti-HRS antibody, either alone or in combination with anti-PD1 or anti-pD-L1 antibodies, has clear activity with a prophylactic and potentially therapeutic impact on cancer growth in this colon cancer model system, as well as the potential to synergize with other anti-cancer therapeutics.

### Example 10

### Combination of PD-L1 and Anti-HRS Antibodies Inhibit Tumor Growth in A Therapeutic B16F10 Syngeneic Tumor Model

This study aims to investigate whether the in vivo anti-cancer potential of anti-HRS antibodies alone or in combination with an anti-PD-L1 antibody in a syngeneic mouse model using B16-F10 cells (mouse melanoma model) prepared as described in Materials and Methods. In this study the dosing regimen (Table E9) was initiated three days after cell injection (i.e., therapeutically). The animal weights and tumor measurements were recorded three times a week.

Treatment Regimen.

Eighty (80) C57bl/6 mice (JAX #000664, female, 5-6 wks old) were used in this study. The animals were assigned to 8 study groups of 10 mice randomly. The B16-F10 cell line was expanded for injection as described in the Materials and Methods. The dosing regimen is shown in Table E9 below; In brief animals received injections of 10 mg/kg of each control IgG, positive control antibodies, and anti-HRS antibodies as described in the Materials and Methods, which was administered to mice intraperitoneally twice weekly (on day 3, 6, 10 and 13 post implant) according to the protocol below (Table E9).

TABLE E9
GROUP TREATMENTS

| | | | Dose | | |
|---|---|---|---|---|---|
| Group | #Mice | Materials | (mg/kg) | ROA | Frequency |
| 1 | 10 | IgG (control) | 20 | IP | Day 3, 6, 10, 13 |
| 2 | 10 | αPD-L1 + | 10 | IP | Day 3, 6, 10, 13 |
| | | IgG (control) | 10 | | |
| 3 | 10 | ATYR13C8 + | 10 | IP | Day 3, 6, 10, 13 |
| | | IgG (control) | 10 | | |
| 4 | 10 | ATYR13E9 + | 10 | IP | Day 3, 6, 10, 13 |
| | | IgG (control) | 10 | | |
| 5 | 10 | αPD-L1 + | 10 | IP | Day 3, 6, 10, 13 |
| | | αPD-1 | 10 | | |
| 6 | 10 | ATYR13C8 + | 10 | IP | Day 3, 6, 10, 13 |
| | | ATYR13E9 | 10 | | |
| 7 | 10 | ATYR13C8 + | 10 | IP | Day 3, 6, 10, 13 |
| | | αPD-L1 | 10 | | |
| 8 | 10 | ATYR13E9 + | 10 | IP | Day 3, 6, 10, 13 |
| | | αPD-L1 | 10 | | |

Summary of Results.

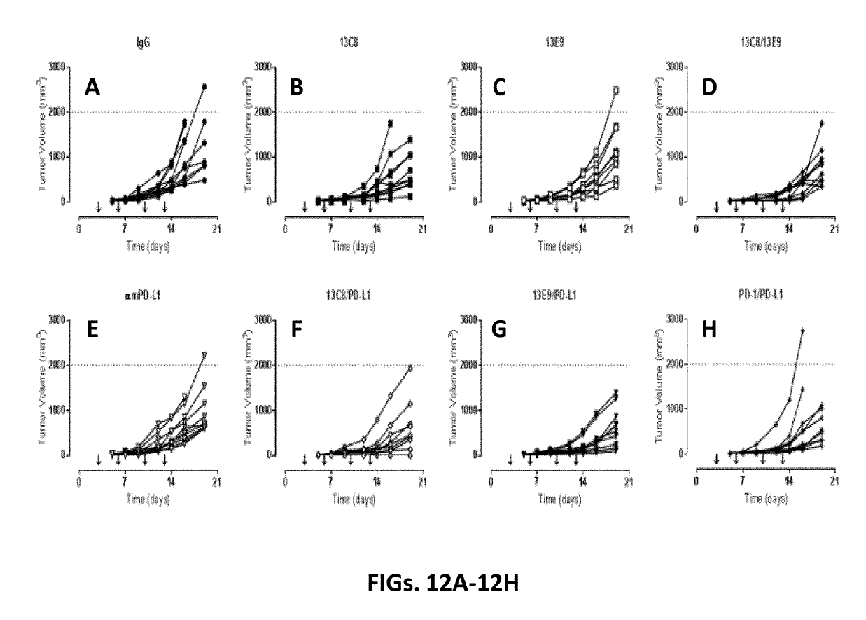

There was no evidence of toxicity from weight measurements and observations during the study (data not shown). Animals bearing B16-F10 tumors treated with 4 doses of either ATYR13E9 or ATYR13C8 alone or together with an anti-PD-L1 antibody in a therapeutic setting (i.e., 3 days after tumor cell inoculation) showed more effective inhibition of tumor growth compared to control IgG beginning at study day 16. (FIGS. 12A-12H). Surprisingly the combination of the n-terminally directed anti-HRS antibody (clone ATYR13E9) or the c-terminally directed anti-HRS antibody (clone ATYR13C8) in combination with an anti-PD-L1 antibody appeared to be somewhat more effective than the anti-PD-L1 antibody alone. The combination of anti-HRS antibodies (i.e., ATYR13E9 plus ATYR13C8) was surprisingly also more effective than either antibody alone.

These results demonstrate that an anti-HRS antibody, and combinations of an n-terminally directed anti-HRS antibody (clone ATYR13E9) and/or the c-terminally directed anti-HRS antibody (clone ATYR13C8) in combination with an anti-PD-L1 have clear potential for a prophylactic and potentially therapeutic impact on cancer growth in this model system, as well as the potential to synergize with other anti-cancer therapeutics, and treat other cancers.

### Example 11

### Combination of Anti-PD-1 and Anti-HRS Antibody ATYR13E9 Inhibit Tumor Growth in the 4T1 Breast Cancer Model More Effectively than Either Antibody Alone

This study aims to investigate whether in vivo anticancer potential of test compounds in a syngeneic mouse model using 4T1 cells. Cells were prepared as described in Materials and Methods. In this study the anti-HRS antibody ATYR13E9 was administered separately and in combination with an anti-PD-1 antibody. The animal weights and tumor measurements were recorded three times a week by a person blind to the treatments.

Treatment Regimen.

One-Hundred (100) Balb/c mice (Envigo, female, 5-6 wks old) were used in this study. The animals were assigned to 10 study groups of 10 mice randomly. The 4T1 cell line was expanded for injection as described in the Materials and Methods. The dosing regimen is shown in Table E10 below; In brief animals received injections of each control IgG, positive control antibodies, and anti-HRS antibodies as described in the Materials and Methods, which was administered to mice intraperitoneally once weekly starting the day prior to cell implantation (Day −1) and on day 6 and 13 post implant according to the protocol below (Table E10). Groups 6-10 were designated to terminate mid-study, groups 1-5 were designed to be followed until euthanasia criteria were met. In animals surviving until Day 63 and bearing tumors, a second regimen of antibody treatment was initiated.

TABLE E10
GROUP TREATMENTS

| | | | Dose | | |
|---|---|---|---|---|---|
| Group | #Mice | Materials | (μg/mouse) | ROA | Frequency |
| 1 | 10 | Naive (no tumor, | NA | NA | NA |
| | | no tx) | | | |
| 2 | 10 | IgG (control) | 200 | IP | D −1, 6, 13 |
| 3 | 10 | ATYR13E9 + | 200 | IP | D −1, 6, 13 |
| | | IgG (control) | 200 | | |
| 4 | 10 | αPD-1 + | 50* | IP | D −1, 6, 13 |
| | | IgG (control) | 200 | | |
| 5 | 10 | αPD-1 + | 50* | IP | D −1, 6, 13 |
| | | ATYR13E9 | 200 | | |
| 6 | | Naive (no tumor, | NA | IP | NA |
| | | no tx) | | | |
| 7 | 10 | IgG (control) | 200 | IP | D −1, 6, 13 |
| 8 | 10 | ATYR13E9 + | 200 | IP | D −1, 6, 13 |
| | | IgG (control) | 200 | | |
| 9 | 10 | αPD-1 + | 50* | IP | D −1, 6, 13 |
| | | IgG (control) | 200 | | |
| 10 | 10 | αPD-1 + | 50* | IP | D −1, 6, 13 |
| | | ATYR13E9 | 200 | | |

*Anti-PD-1 antibody was administered at 50 μg/mouse on Days −1 and 6; at 200 μg/mouse on all other dosing days

Summary of Results.

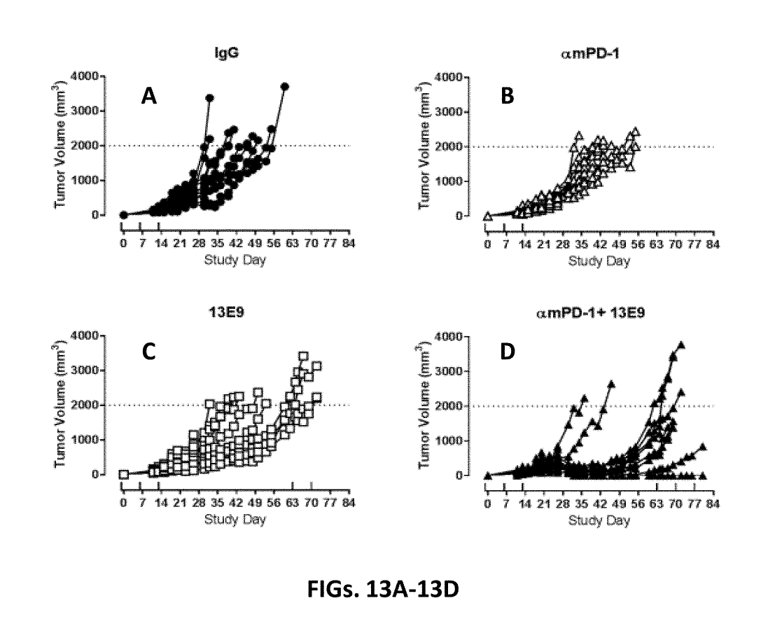

There was no evidence of toxicity from body weight measurements and observations during the study (data not shown). Animals bearing 4T1 tumor treated with 3 doses of ATYR13E9 alone or together with an anti-PD1 antibody showed more effective inhibition of tumor growth compared to control IgG beginning at study day 30. (FIGS. 13A-13D). Animals in which tumors had re-grown receiving additional antibody intervention on Day 63 showed no change in the trajectory of their tumor growth. These results demonstrate that an anti-HRS antibody in combination with an anti-PD-1 antibody have clear potential for a prophylactic and potentially therapeutic impact on cancer growth in this melanoma model system, as well as the potential to synergize with other anti-cancer therapeutics.

### Example 12

### Combination of Anti-PD-L1 or Anti-PD-1 and Anti-HRS Antibodies ATYR13E9 and ATYR13C8 Inhibit Tumor Growth in the Pan02 Pancreatic Cancer Model More Effectively than any Antibody Alone

This study aims to investigate whether in vivo anticancer potential of test compounds in a syngeneic mouse model using Pan02 cells (a pancreatic cancer model). Cells were prepared as described in Materials and Methods. In this study the anti-HRS antibodies ATYR13E9 and ATYR13C8 were administered separately and in combination with an anti-PD-1 or anti-PD-L1 antibodies to extend the results to a fourth model and determine whether there were any functional consequences of blocking HRS on the growth of this tumor cell line or on the anti-cancer properties of the PD-1 or PD-L1 antibodies. The dosing regimen was initiated one day before cell injection. The animal weights and tumor measurements were recorded three times a week by a person blind to the treatments.

Treatment Regimen.

One-Hundred (100) C57bl/6 (Envigo, female, 5-6 wks old) were used in this study. The animals were assigned to 10 study groups of 10 mice randomly. The Pan02 cell line was expanded for injection as described in the Materials and Methods. The dosing regimen is shown in Table E11 below; In brief animals received injections of each control IgG, positive control antibodies, and anti-HRS antibodies as described in the Materials and Methods, which was administered to mice intraperitoneally once weekly starting the day prior to cell implantation (Day −1) and on day 6 and 13 post implant according to the protocol below (Table E11). Groups were designed to be followed until euthanasia criteria were met. The last animal was terminated on Day 66.

TABLE E11
GROUP TREATMENTS

| | | | Dose | | |
|---|---|---|---|---|---|
| Group | #Mice | Materials | (mg/kg) | ROA | Frequency |
| 1 | 10 | Control IgG1 | 10 | IP | D −1, 6, 13 |
| 2 | 10 | αmPD-L1 | 10 | IP | D −1, 6, 13 |
| 3 | 10 | ATYR13C8 | 10 | IP | D −1, 6, 13 |
| 4 | 10 | ATYR13E9 | 10 | IP | D −1, 6, 13 |
| 5 | 10 | αmPD-1 | 10 | IP | D −1, 6, 13 |
| 6 | 10 | αmPD-L1 + | 10 | IP | D −1, 6, 13 |
| | | ATYR13C8 | 10 | | |
| 7 | 10 | αmPD-L1 + | 10 | IP | D −1, 6, 13 |
| | | ATYR13E9 | 10 | | |
| 8 | 10 | αmPD-1 + | 10 | IP | D −1, 6, 13 |
| | | ATYR13C8 | 10 | | |
| 9 | 10 | αmPD-1 + | 10 | IP | D −1, 6, 13 |
| | | ATYR13E9 | 10 | | |
| 10 | 10 | No tumor, no | NA | NA | NA |
| | | treatment | | | |

Summary of Results.

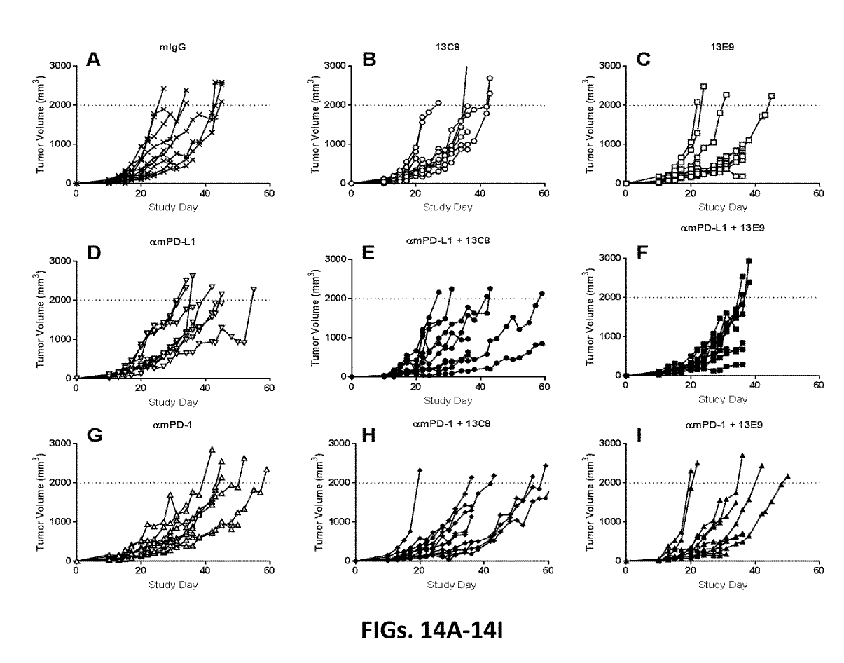

There was no evidence of toxicity from body weight measurements and observations during the study (data not shown). Animals bearing Pan02 tumors treated with 3 doses of ATYR13E9 or ATYR13C8 together with an anti-PD-L1 or an anti-PD-1 antibody tended to show more effective inhibition of tumor growth compared to control IgG in that several animals treated with the combinations had slowed tumor growth to day 40 (FIGS. 14A-14I). These results demonstrate that an anti-HRS antibody in combination with an anti-PD-1 or an anti-PD-L1 antibody have clear activity with prophylactic and potentially therapeutic impact on cancer growth in this pancreatic cancer model system, as well as the potential to synergize with other anti-cancer therapeutics, and treat other cancers.

### Example 13

### Combination of Indoleamine 2, 3-Dioxygenase-1 (IDO) Inhibition and Anti-Hrs Antibody ATYR13E9 Regresses Tumors in CT26 Colon Cancer Model More Effectively than Either Alone

This study aims to investigate the in vivo anticancer potential of test compounds in a syngeneic mouse model using CT26 cells. Cells were prepared as described in Materials and Methods. In this study the anti-HRS antibody ATYR13E9 was administered separately and in combination with a small molecule inhibitor of indoleamine 2, 3-dioxygenase-1 (IDOi) to determine whether there were any functional consequences of blocking HRS on the anti-cancer properties of the IDOi. The dosing regimen was initiated after animals were randomized to study based on tumor volumes. The animal weights and tumor measurements were recorded three times a week by a person blind to the treatments.

Treatment Regimen.

Ninety (90) Balb/c mice (Envigo, female, 5-6 wks old) were used in this study. For brevity, this example focuses on 4 study groups of 10 mice which were randomized to study groups 8 days after implantation of CT26 cells based on tumor volumes (mean=118 mm³) which were expanded for injection as described in the Materials and Methods. The dosing regimen is shown in Table E12 below; In brief animals received injections of each control IgG and anti-HRS antibody as described in the Materials and Methods. Starting 8 days post implant, anti-HRS antibody was administered to mice intraperitoneally twice weekly and IDOi was administered twice daily (BID) for a period of 3 weeks according to the protocol below (Table E12). Groups were designed to be followed until euthanasia criteria were met.

TABLE E12
GROUP TREATMENTS

| | | | Dose | | |
|---|---|---|---|---|---|
| | | | (mg/ | | |
| Group | #Mice | Materials | kg) | ROA | Frequency |
| 1 | 10 | Control IgG1 + | 10 | IP | D8, 11, 15, 18, 22, 25 |
| | | Vehicle | 0 | PO | BID Day 8-term |
| 2 | 10 | ATYR13E9 + | 10 | IP | D8, 11, 15, 18, 22, 25 |
| | | Vehicle | 0 | PO | BID Day 8-term |
| 3 | 10 | Control IgG1 | 10 | IP | D8, 11, 15, 18, 22, 25 |
| | | IDOi | 100 | PO | BID Day 8-term |
| 4 | 10 | ATYR13E9 + | 10 | IP | D8, 11, 15, 18, 22, 25 |
| | | IDOi | 100 | PO | BID Day 8-term |

Summary of Results.

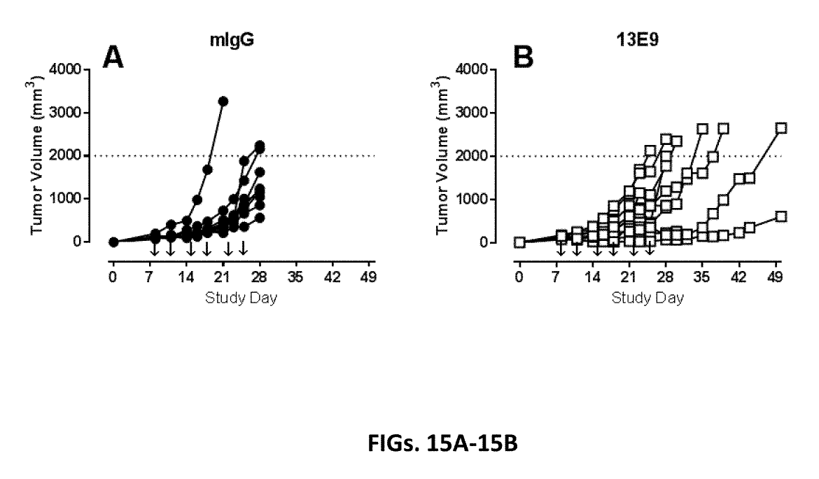

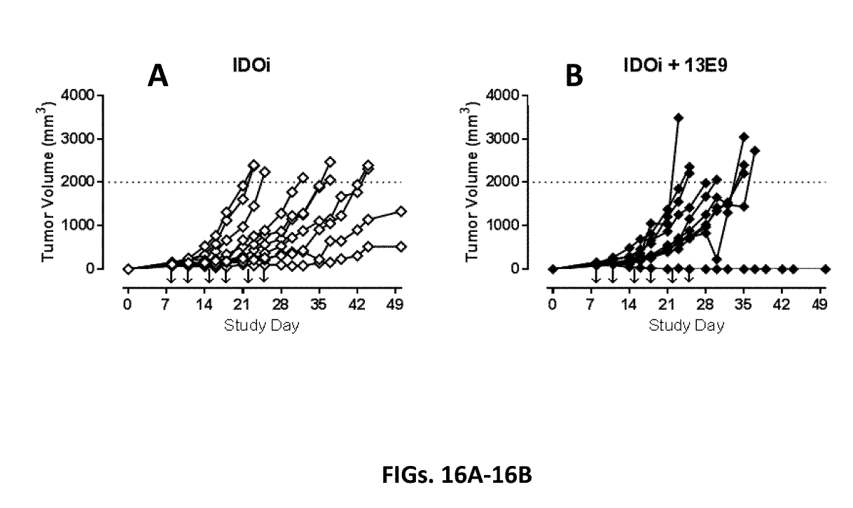

There was no evidence of toxicity from body weight measurements and observations during the study (data not shown). Animals bearing CT26 tumors treated with 6 doses of ATYR13E9 together with an IDO inhibitor show that 1 animal treated with the combination had complete tumor regression, suggesting that at least in some animals the combination was more effective inhibiting tumor growth than either agent alone (FIGS. 15A-15B and FIGS. 16A-16B). These results demonstrate that an anti-HRS antibody in combination with an IDOi has a clear therapeutic impact on cancer growth in this colon cancer model system as well as the potential to combine with other anti-cancer therapeutics.

### Example 14

### Prior Exposure to a Tumor Enhances the Tumor Response to Combined Anti-Hrs Antibodies ATYR13C8 and ATYR13E9 in B16F10 Melanoma Model

This study aims to investigate the in vivo anticancer potential of test compounds in a syngeneic mouse model using B16F10 cells. Cells were prepared as described in Materials and Methods. Animals were exposed to tumors which were surgically removed (“tumor exposed”) or left naive (“tumor naive”; detailed in Materials and Methods). In this study the anti-HRS antibodies ATYR13E9 and ATYR13C8 were administered together to determine whether there were any functional consequences of blocking HRS on the response to a tumor type to which the mouse's immune system had been previously exposed. Anti-mouse-PD-1 and anti-mouse-PD-L1 antibodies were included for comparison. The dosing regimen was initiated the day before implantation of the test tumor. The animal weights and tumor measurements were recorded three times a week.

Treatment Regimen.

Eighty (80) C57bl/6 mice (Jax, female, 5-6 wks old) were used in this study. The dosing regimen is shown in Table E13 below; In brief animals received injections of each control IgG, anti-HRS antibody, anti-PD-1 antibody, and anti-PD-L1 antibody as described in the Materials and Methods. Starting the day before test tumor implant, anti-HRS antibody was administered to mice intraperitoneally once weekly according to the protocol below (Table E13). Groups were designed to be followed until euthanasia criteria were met.

TABLE E13
GROUP TREATMENTS

| | | Tumor | Test Tumor | | Dose | | |
|---|---|---|---|---|---|---|---|
| Group | #Mice | (Day −26) | (Day 0) | Materials | (mg/kg) | ROA | Frequency |
| 1 | 12 | B16F10 | B16F10 | Control IgG1 | 20 | IP | Day −1, 6, 13 |
| 2 | 12 | B16F10 | B16F10 | αmPD-L1 + | 10 | IP | Day −1, 6, 13 |
| | | | | αmPD-1 | 10 | IP | |
| 3 | 12 | B16F10 | B16F10 | ATYR13E9 + | 10 | IP | Day −1, 6, 13 |
| | | | | ATYR13C8 | 10 | IP | |
| 4 | 12 | None | B16F10 | Control IgG1 | 20 | IP | Day −1, 6, 13 |
| 5 | 12 | None | B16F10 | αmPD-L1 + | 10 | IP | Day −1, 6, 13 |
| | | | | αmPD-1 | 10 | IP | |
| 6 | 12 | None | B16F10 | ATYR13E9 + | 10 | IP | Day −1, 6, 13 |
| | | | | ATYR13C8 | 10 | IP | |

Summary of Results.

There was no evidence of toxicity from body weight measurements and observations during the study (data not shown). Tumor naive older mice in this study did not have significant tumor growth inhibition responses to the combination of anti-mouse-PD-L1 and anti-mouse-PD-1 or the combination of anti-HRS antibody ATYR13C8 and ATYR13E9 in contrast to previous studies (data not shown). However, there was significant inhibition of test tumor growth in previously tumor exposed mice treated with the combination or anti-mouse-PD-L1 and anti-mouse-PD-1 or the combination of anti-HRS antibodies ATYR13C8 and ATYR13E9 (p<0.05, 2-way ANOVA followed by Dunnett's post-hoc test), suggesting that the immune response, primed by previous exposure to tumor (and associated antigens), is enhanced by HRS blockade and demonstrate that an anti-HRS antibody has a clear potential for a prophylactic and potentially therapeutic impact on cancer growth in this melanoma model system, as well as the potential to synergize with other anti-cancer therapeutics.

### Example 15

### Anti-Cancer Effects of Anti-HRS Antibodies Depends on Presence of CD8+ T Cells and NK1.1+ Natural Killer Cells

This study aims to investigate in vivo anticancer potential of test compounds in a syngeneic mouse model using B16F10 cells in animals depleted of CD8+ T cells, CD4+ T cells or NK1.1+ natural killer (NK) cells. B16F10 cells were prepared as described in Materials and Methods. In this study the anti-HRS antibodies ATYR13E9 and ATYR13C8 were administered in combination to determine whether there were any differences in the anti-cancer properties of HRS binding in the absence of specific immune cell types. The dosing regimens were initiated one day before cell injection. The animal weights and tumor measurements were recorded three times a week.

Treatment Regimen.

Forty (40) C57bl/6 mice (Jax, female, 5-6 wks old) were used in this study. The animals were assigned to 4 study groups of 10 mice randomly. The B16F10 cell line was expanded for injection as described in the Materials and Methods. The dosing regimen is shown in Table E14 below; In brief all animals received intraperitoneal injections of anti-HRS antibodies as described in the Materials and Methods, and were specifically depleted of each immune cell type beginning the day before tumor implant according to the protocol below (Table E14).

TABLE E14
GROUP TREATMENTS

| | | | | | | Dose | |
|---|---|---|---|---|---|---|---|
| | | | Dose | | | (μg/ | |
| Group | #Mice | Materials | (mg/kg) | Frequency | Material | mouse) | Frequency |
| 1 | 10 | ATYR13C8 + | 10 | Day −1, 6, 13 | IgG2b | 100 | Day −1, 3, |
| | | ATYR13E9 | 10 | | | | 6, 10, 13 |
| 2 | 10 | ATYR13C8 + | 10 | Day −1, 6, 13 | Anti-CD8 | 100 | Day −1, 3, |
| | | ATYR13E9 | 10 | | | | 6, 10, 13 |
| 3 | 10 | ATYR13C8 + | 10 | Day −1, 6, 13 | Anti-CD4 | 100 | Day −1, 3, |
| | | ATYR13E9 | 10 | | | | 6, 10, 13 |
| 4 | 10 | ATYR13C8 + | 10 | Day −1, 6, 13 | Anti- | 100 | Day −1, 3, |
| | | ATYR13E9 | 10 | | NK1.1 | | 6, 10, 13 |

Summary of Results.

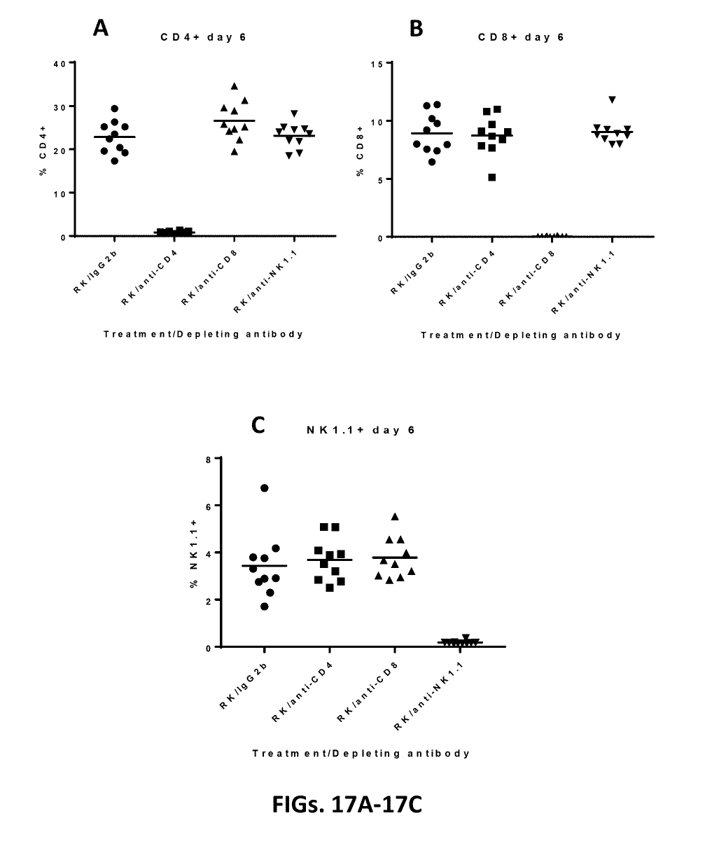

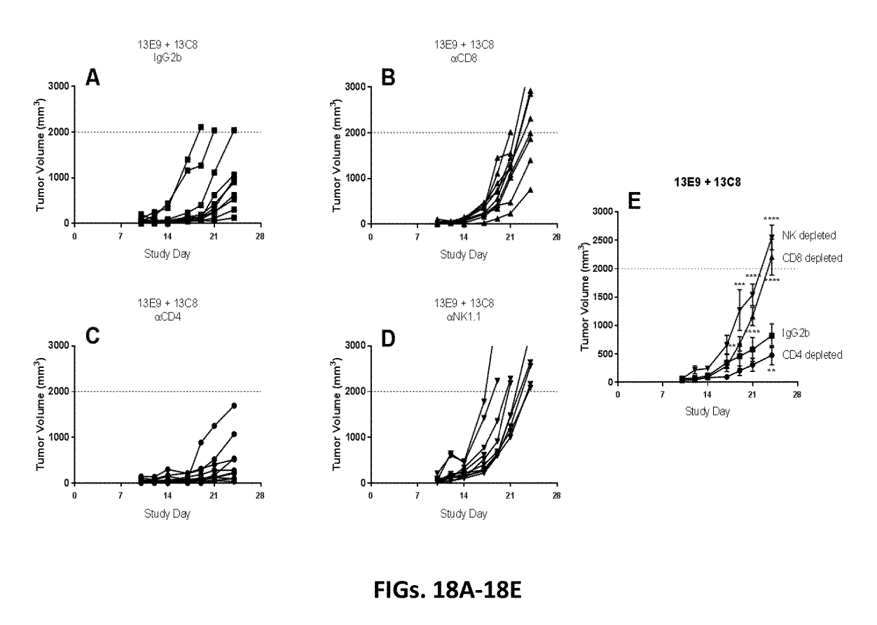

There was no evidence of toxicity from body weight measurements and observations during the study (data not shown). Flow cytometry conducted on Day 6 confirmed that anti-CD4, anti-CD8 and anti-NK1.1 antibodies specifically depleted the targeted immune cells (FIGS. 17A-17C). Animals bearing B16F10 tumors and treated with 3 doses of ATYR13E9 combined with ATYR13C8 and receiving control IgG2b showed modest tumor growth (FIG. 18A-18E). Animals receiving anti-HRS antibodies and depleted of CD4+ T cells had slower tumor growth, potentially due to depletion of CD4+ regulatory T cells which can promote tumor growth. Animals receiving anti-HRS antibodies and depleted of either CD8+ T cells or NK1.1+NK cells had dramatically increased tumor growth, suggesting that the anti-cancer activity of anti-HRS antibodies depends on these cell types. These results demonstrate that an anti-HRS antibody has an immune-based mechanism and have clear activity with a prophylactic and potentially therapeutic impact on cancer growth in this melanoma model system, as well as the potential to synergize with other anti-cancer therapeutics.

### Example 16

### Evaluation of Anti-Tumor Activity of Test Compounds on B16-F10 Mouse Melanoma in C57BL/6 Mice

This study was designed to investigate the in vivo anticancer potential of test compounds in a syngeneic mouse model using B16-F10 cells (mouse melanoma cancer) prepared as described in Materials and Methods. The test antibody dosing regimen was initiated one day before cell injection and animal weights and tumor measurements were recorded three times a week until study termination.

Treatment Regimen.

Ninety (90) C57BL/6 mice (Jax, female, 5-6 wks old) were used in this study. For brevity, this example focuses on 4 treatment groups. The animals were assigned to study groups of 10 mice randomly, and housed as described in the Materials and Methods. The antibody dosing regimen is shown in Table E15 below; in brief animals received intraperitoneal injections of 10 mg/kg of each of the control IgG antibodies, positive control αPD-L1 antibody, and anti-HRS antibodies 13E9 or KL31-600 (as described in the Materials and Methods), administered according to the protocol below (Table E15); starting one day before cancer cell implantation and then on day 6 and 13 post-cell implantation.

TABLE E15
GROUP TREATMENTS

| | | | | Route of | |
|---|---|---|---|---|---|
| | | | Dose | Administration | |
| Group | #Mice | Test Materials | (mg/kg) | (ROA) | Frequency |
| 1 | 10 | Control IgG | 20 | IP | days −1, |
| | | | | | 6, 13 |
| 2 | 10 | αPD-L1 + | 10 | IP | days −1, |
| | | Control IgG | 10 | | 6, 13 |
| 3 | 10 | 13E9 + | 10 | IP | days −1, |
| | | Control IgG | 10 | | 6, 13 |
| 4 | 10 | KL31-600 + | 10 | IP | days −1, |
| | | Control IgG | 10 | | 6, 13 |

Summary of Results.

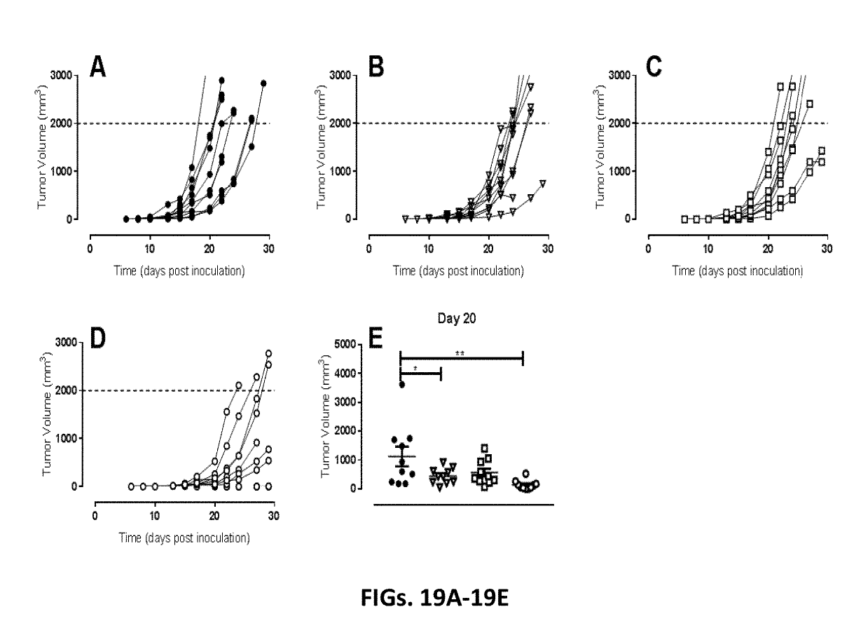

Animals bearing B16-F10 tumors and treated with 3 doses of 13E9 or KL31-600 showed reduced tumor growth, reaching statistical significance (p≤0.01) on Day 20, the last day all animals were on study (FIG. 19A-19D). Animals bearing B16-F10 tumors and treated with 3 doses of anti-mouse PD-L1 also showed reduced tumor growth (p≤0.05 on Day 20). Surprisingly the N-terminally directed KL31-600 inhibited B16-F10 Melanoma growth better than the bench mark anti-mouse PD-L1 (p<0.01; FIG. 19E shows day 20 data). There was no evidence of toxicity from animal body weight measurements and observations during the study (data not shown). These results demonstrate that anti-HRS antibodies have clear activity with a prophylactic and potentially therapeutic impact on cancer growth in this model system.

### Example 17

### Anti-HRS Antibodies Inhibit Tumor Growth and Enhance Tumor Growth Inhibition in Combination with PD-L1 Pathway Blockade in the CT26 Tumor Model

This study aims to investigate the in vivo anti-cancer potential of anti-HRS antibodies alone or in combination with anti-PD-L1 antibodies in a syngeneic mouse model using CT26 cells (mouse colon cancer) prepared as described in Materials and Methods. In this study the dosing regimen (Table E16) was initiated one day before cell injection (i.e., prophylactically). The animal weights and tumor measurements were recorded three times a week.

Treatment Regimen.

Mice were randomly assigned to 10 per group. The CT26 cell line was expanded for injection as described in the Materials and Methods. The dosing regimen is shown in Table E16 below; In brief animals received injections of 10 mg/kg of each control IgG, positive control antibodies, and anti-HRS antibodies as described in the Materials and Methods, which were administered to mice intraperitoneally starting one day before cancer cell implantation and on Days 6 and 13 post implantation) according to the protocol below (Table E16).

TABLE E16
GROUP TREATMENTS

| | | | Dose | | |
|---|---|---|---|---|---|
| Group | #Mice | Materials | (mg/kg) | ROA | Frequency |
| 1 | 10 | hIgG1 + | 10 | IP | Days −1, 6, 13 |
| | | rIgG2b | 10 | | |
| 2 | 10 | hIgG1 + | 10 | IP | Days −1, 6, 13 |
| | | αmPD-L1 | 10 | | |
| 3 | 10 | 13E9 + | 10 | IP | Days −1, 6, 13 |
| | | rIgG2b | 10 | | |
| 4 | 10 | KL31-241 + | 10 | IP | Days −1, 6, 13 |
| | | rIgG2b | 10 | | |
| 5 | 10 | 13E9 + | 10 | IP | Days −1, 6, 13 |
| | | αmPD-L1 | 10 | | |
| 6 | 10 | KL31-241 + | 10 | IP | Days −1, 6, 13 |
| | | αmPD-L1 | 10 | | |
| 7 | 10 | No treatment | | | |
| 8 | 10 | No tumor, no | | | |
| | | treatment | | | |

Summary of Results.

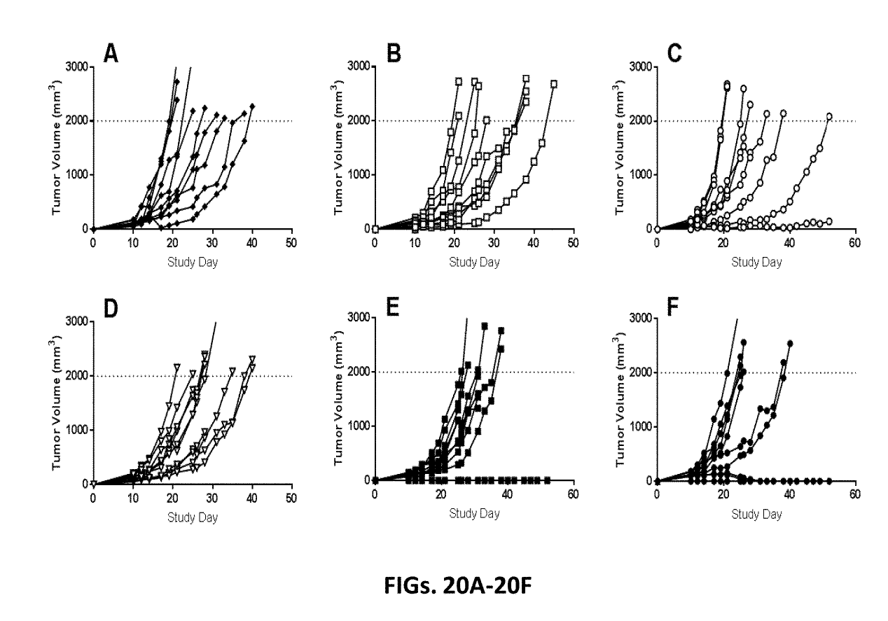

There was no evidence of toxicity from weight measurements and observations during the study (data not shown). Animals bearing CT26 tumors treated with 3 doses of anti-HRS antibodies together with an anti-PD-L1 antibody showed more effective inhibition of tumor growth compared to tumor-bearing untreated controls beginning at study day 19 (FIGS. 20A-20F). Surprisingly, KL31-241 showed a strong tendency to inhibit tumor growth as a monotherapy. Furthermore, mice treated with HRS binding antibodies had a least 1 animal that never grew a tumor with prophylactic treatment. In fact, KL31-241 caused tumor regression in 20% of the treated animals in combination with anti-PD-L1, the only treatment regimen to cause such a dramatic response.

These results demonstrate that an anti-HRS antibody, alone and in combination with anti-PD-L1 blockade have clear activity with a prophylactic and potentially therapeutic impact on cancer growth in this colon cancer model system as well as the potential to combine with other anti-cancer therapeutics.

### Example 18

### In Contrast to Anti-PD-L1 Antibodies, Anti-HRS Antibodies do not Precipitate Type 1 Diabetes in Female NOD Mice

This study aims to investigate the in vivo type 1 diabetes induction potential of an anti-HRS antibodies in comparison to anti-PD-L1 antibodies in NOD mice prepared as described in Materials and Methods. In this study the dosing regimen (Table E17) was twice weekly for two weeks. The animal weights and glucose levels were recorded four times a week.

Treatment Regimen.

One hundred (100) NOD mice (Jax, female, 11 wks old) were used in this study. The animals were assigned to 7 study groups of 10-15 mice randomly (see Materials and Methods). The dosing regimen is shown in Table E17 below. In brief animals received injections of 10 mg/kg of each control IgG, anti-mouse PD-L1, and anti-HRS antibodies as described in the Materials and Methods, which were administered to mice intraperitoneally on days 0, 4, 7, and 11 according to the protocol below (Table E17). Animals were euthanized on Day 15 or earlier if glucose levels exceeded 750 mg/dL or animals were moribund.

TABLE E17
GROUP TREATMENTS

| | | | Dose | | |
|---|---|---|---|---|---|
| Group | #Mice | Materials | (mg/kg) | ROA | Frequency |
| 1 | 10 | Naive | | | |
| 2 | 15 | rIgG2b | 10 | IP | Days 0, 4, 7, 11 |
| 3 | 15 | Anti-mouse PD- | 10 | IP | Days 0, 4, 7, 11 |
| | | LI (rIgG2b) | | | |
| 4 | 15 | mIgG1 | 10 | IP | Days 0, 4, 7, 11 |
| 5 | 15 | 13E9 (mIgG1) | 10 | IP | Days 0, 4, 7, 11 |
| 6 | 15 | hIgG1 | 10 | IP | Days 0, 4, 7, 11 |
| 7 | 15 | KL31-241 | 10 | IP | Days 0, 4, 7, 11 |
| | | (hIgG1) | | | |

Summary of Results.

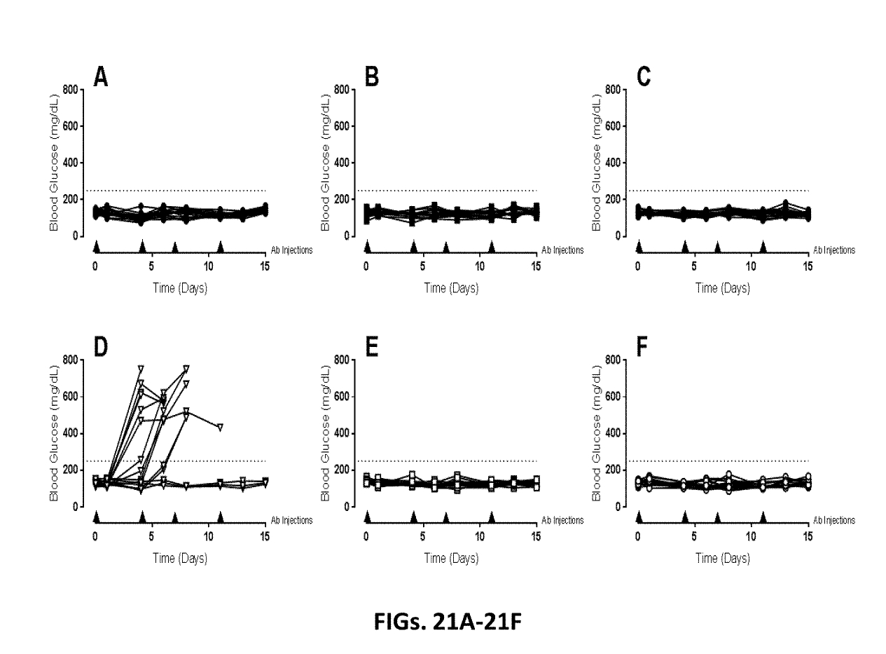

Seventy-five percent (75%) of NOD mice receiving an anti-PD-L1 antibody developed hyperglycemia/diabetes by study Day 8 (FIGS. 21A-21F). Surprisingly, anti-HRS antibodies 13E9 and KL31-241 did not precipitate diabetes in the NOD mice.

These results demonstrate that an anti-HRS antibody does not mimic an anti-PD-L1 antibody in driving autoimmune disease, suggesting that anti-HRS has a distinct mechanism of action and the potential for an improved safety profile.

### Example 19

### Initial Receptor Identification Screen

To identify potential interacting partners of HRS, and related HRS polypeptides, the Retrogenix cell microarray screening technology (Retrogenix Ltd., High Peak Rd, United Kingdom) was used to evaluate binding of a HRS-Fc fusion protein construct ([Fc-HRS(2-60)] to a library of approximately 4500 membrane bound human proteins individually expressed in HEK293 cells.

In brief, HEK293 cells were plated onto glass cover slides which have been pre-treated by the application of discrete expression vectors to enable reverse transfection and expression of each of the 4500 membrane proteins, to create a cell microarray. Transfection efficiencies were assessed via ZsGreen1 expression and exceeded the minimum threshold for all of the library members screened.

Use of a smaller HRS fragment with a Fc tag provided for high sensitivity detection via the use of an AlexaFluor647 labeled anti-human IgG Fc antibody (AF647) as the detection reagent, and readily available controls to confirm specificity. The detection antibody was used at screening concentrations of 2, 5, and 20 μg/ml, as more fully described below.

Test Protein was screened at a concentration of 20 μg/ml using two different screening formats; either a sequential staining method, or a pre-incubation staining method. Sequential staining involved, in brief, the sequential addition to the test cells, of the test protein and detection reagents, while the pre-incubation staining method involved the pre-incubation of the test protein with the detection reagents (2:1 molar ratio of test protein to detection antibody) to pre-form higher avidity complexes prior to the addition to the test cells. A background screen was completed with test protein added to slides of fixed, untransfected HEK293 cells to confirm that the test protein did not bind to untransfected cells.

Primary hits (duplicate spots) were identified by analyzing fluorescence intensity in the AlexaFluor 647 and ZsGreen1 emission channels using the ImageQuant system, following standard fluorescent methodology. Confirmation Screens were run to evaluate any screening hits identified from the primary screen, using identical fixed slides treated with 20 μg/ml test protein, or positive or negative controls, using the sequential or pre-incubation methods (n=2 slides per treatment). Additionally all vectors encoding all hits, plus control vectors, were spotted in duplicate on new slides, and used to reverse transfect human HEK293 cells as before. All transfection efficiencies exceeded the minimum threshold Hits were categorized as specific, or non-specific (i.e. it also came up with at least one of the positive or negative controls), and if specific whether the hit was strong, medium or weak binding. Confirmatory hits using both the sequential and pre-incubation staining methods are summarized in Table E18 and Table E19 below.

TABLE E18
Sequential incubation confirmatory screening result summary

| Hit | | | | | | |
|---|---|---|---|---|---|---|
| No. | Gene ID: | Accession # | Fc-HRS | CTLA4-FC | RITUXIMAB | PBS |
| 1 | FCGR1A | | NON SPEC | NON SPEC | NON SPEC | NON SPEC |
| 2 | SLC13A3 | | INVERSE | NON SPEC | INVERSE | N/A |
| 3 | NRP2A | NM_003872.2 | WEAK/MED | N/A | N/A | N/A |
| 4 | IGHG3 | | STRONG | STRONG | STRONG | STRONG |
| 5 | FCGR2A | | NON SPEC | NON SPEC | NON SPEC | N/A |
| 6 | NRP2B | NM_201267.1 | WEAK/MED | N/A | N/A | N/A |
| 7 | MS4A1 | | N/A | N/A | STRONG | N/A |
| 8 | CD86 | | N/A | STRONG | N/A | N/A |
| 9 | EGFR | | N/A | N/A | N/A | N/A |
| 10 | SLC38A2 | | WEAK | N/A | N/A | N/A |
| 11 | SLC38A4 | | WEAK | N/A | N/A | N/A |
| 12 | COLEC12 | | WEAK | N/A | N/A | N/A |

TABLE E19
Pre-incubation confirmatory screening result summary

| Hit | | | Binding scoring | | |
|---|---|---|---|---|---|
| No. | Gene ID: | Accession # | Fc-HRS | CTLA4-FC | RITUXIMAB |
| 1 | FCGR1A | | NON SPEC | NON SPEC | NON SPEC |
| 2 | SLC13A3 | | INVERSE | NON SPEC | INVERSE |
| 3 | NRP2 | NM_003872.2 | MED/STRONG | N/A | N/A |
| 4 | IGHG3 | | NON SPEC | NON SPEC | NON SPEC |
| 5 | FCGR2A | | NON SPEC | NON SPEC | NON SPEC |
| 6 | NRP2 | NM_201267.1 | MED/STRONG | N/A | N/A |
| 7 | MS4A1 | | N/A | N/A | STRONG |
| 8 | CD86 | | N/A | STRONG | N/A |
| 9 | EGFR | | N/A | N/A | N/A |
| 10 | SLC38A2 | | WEAK | N/A | N/A |
| 11 | SLC38A4 | | WEAK | N/A | N/A |
| 12 | COLEC12 | | WEAK | N/A | N/A |

Summary/Conclusions.

After screening test protein (Fc-HRS) for binding against 4500+ human plasma membrane proteins expressed in human HEK293 cells, using two incubation approaches, two neuropilin 2 (NRP2) isoforms—(Neuropilin 2A and 2B) were identified as convincing and specific binding partners (using both incubation approaches). The sequential method also identified three, weak intensity hits: SLC38A2, SLC38A4 and COLEC12. These may also be of biological relevance to HRS polypeptides in general and in particular to those comprising the N-terminal domain (1-60) of HRS. Given the broad involvement of the Neuropilin 2 in a broad range of biological processes, including for example, immune activation, immune cell migration, cancer growth, motility and metastasis, lymphogenesis, epithelial-mesenchymal transition (EMT) and nerve fiber growth guidance, these results suggest that HRS, and related HRS polypeptides have the potential to play key regulatory roles in normal and pathophysiology.

### Example 20

### Confirmation of Binding Specificity by SPR Analysis and Identification and Use of Specific Epitopes

Studies were performed to confirm the binding specificity of Neuropilin 2 (NRP2) to Fc-HRS(2-60) using orthologous methods to those used in the large scale Retrogenix screening (Example 19). In a series of experiments, Fc-HRS(2-60) and related proteins were immobilized on SPR chips, and NRP2 and related proteins were flowed as analytes. Upon confirmation of the NRP2:Fc-HRS(2-60) interaction, the dependence on divalent cations was tested as NRP2 is known to have Ca²⁺ binding sites. The effects of previously characterized NRP2 ligands on the NRP2: Fc-HRS(2-60) interaction was also tested to determine if these known ligands have competitive effects on the Fc-HRS(2-60) interaction.

In another series of experiments, monoclonal antibodies (mAbs) which recognize Fc-HRS(2-60) were immobilized on SPR chips. Fc-HRS(2-60) and NRP2 were pre-incubated and injected over the mAb surfaces to determine if only Fc-HRS(2-60), or a larger NRP2: Fc-HRS(2-60) complex was capable of binding to the mAbs. Additionally, co-injection experiments were carried out in which sequential analyte injections of Fc-HRS(2-60) followed by NRP2 were performed. As the different mAbs bind to different epitopes on Fc-HRS(2-60), the ability of the mAbs to bind to an NRP2: Fc-HRS(2-60) complex as opposed to only binding free Fc-HRS(2-60) gives indications as to the interaction surface between the two proteins.

Results.

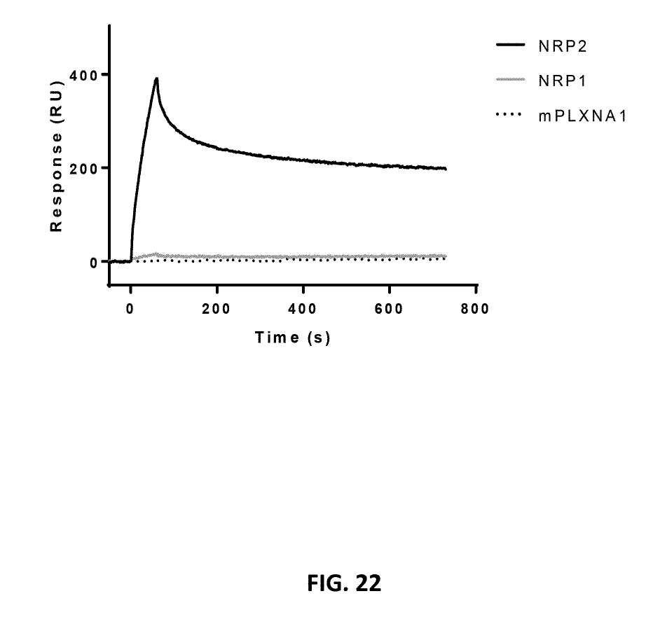

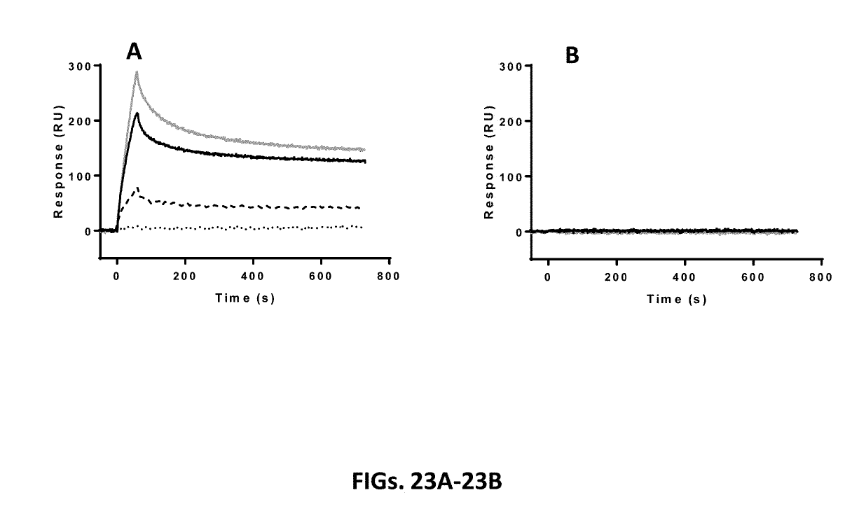

NRP2 but not the closely related NRP1 protein, nor the mouse version of the Plexin A1 co-receptor bind to immobilized Fc-HRS(2-60) (FIG. 22). In addition to human NRP2, both mouse and rat NRP2 demonstrate binding to ATYR1923. However, none of these NRP2 forms bind to a truncated form of Fc-HRS(2-60) with a 49 amino acid deletion at the C-terminus the fusion protein ([Fc-HRS(2-11)] which deleted the majority of the WHEP domain (FIGS. 23A-23B).

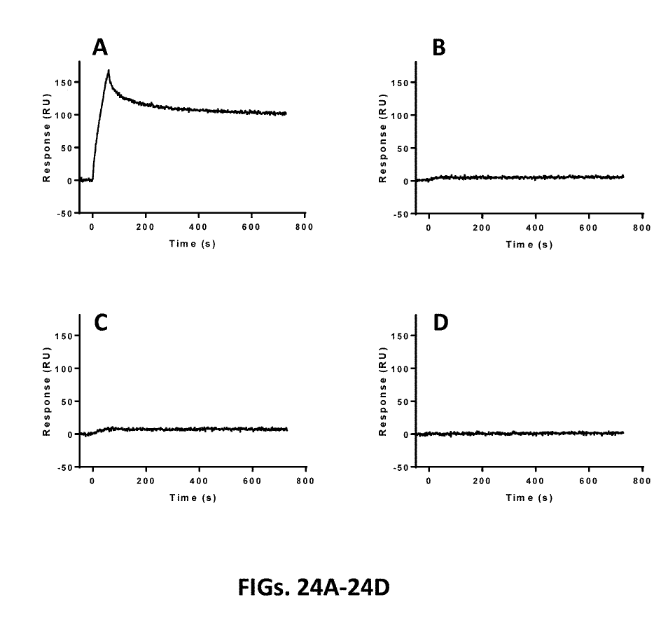

Fc-HRS(2-60) consists of a human IgG Fc region fused to the WHEP domain from histidyl-tRNA synthetase (HRS). Homologous WHEP domains are found in several other tRNA synthetases, including for example, WARS, GARS, MARS, and EPRS. While NRP2 binds to Fc-HRS(2-60), it does not bind to similar proteins consisting of Fc domain fused to the WHEP domain of either GARS or MARS (FIGS. 24A-24D). Additionally NRP2 does not bind to the WHEP domain of WARS with a V5/His tag, suggesting that this interaction with NRP-2 is specific to the HRS WHEP domain and not generally applicable to the other WHEP domains tested.

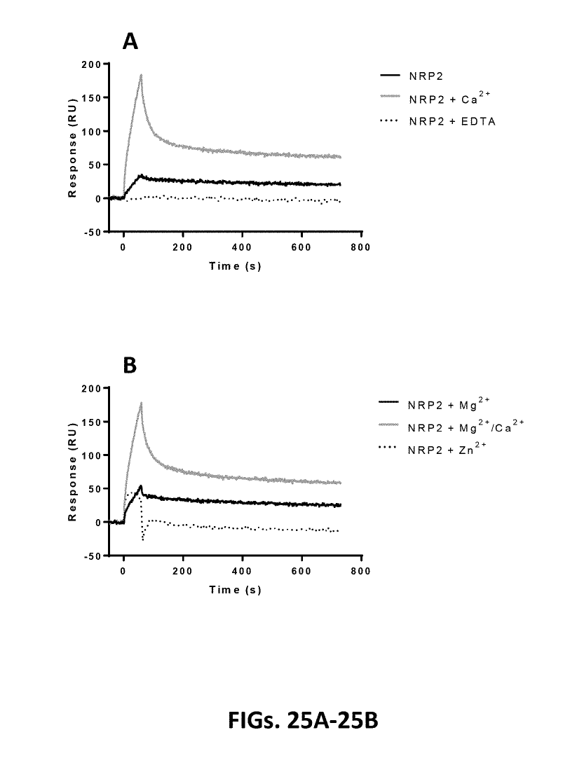

NRP2 is known to have calcium binding sites in its two CUB-domains (a1 and a2 domains). The running buffer of the SPR instrument was switched to a calcium-free buffer (50 mM HEPES, 300 mM NaCl, 0.005% Tween 20, pH 7.4), and CaCl₂, MgCl₂, ZnCl₂ or EDTA were added to the analytes prior to injection and flowed over immobilized Fc-HRS(2-60) (FIGS. 25A-25B). Slight binding was observed in the running buffer alone, while the addition of CaCl₂ greatly enhanced binding. Conversely, addition of ZnCl₂ or EDTA (which chelates divalent cations), resulted in no significant additional binding. Additionally, MgCl₂ up to the concentrations tested, does not appear to have any significant effects on the binding. This result suggests the involvement of the a1 or a2 domains of NRP2 in the interaction with Fc-HRS(2-60) either directly or through maintenance of the conformation of the NRP-2 molecule.

A subset of the known ligands of NRP2 appears to compete binding of Fc-HRS(2-60) to NRP2. The VEGF family of ligands that bind NRP2 appear to prevent binding of Fc-HRS(2-60), while the SEMA family of ligands do not appear to compete binding under the conditions tested (Table E20). In the presence of either VEGF-C, VEGF-A₁₆₅, or PlGF-2/Heparin, binding of NRP2 to Fc-HRS(2-60) is reduced or ablated. Conversely, in the presence of VEGF-A₁₄₅ (which has been reported to be an NRP2 ligand but does not bind NRP2 in our system) or VEGF-A₁₂₁ (which does not bind NRP2), binding of NRP2 to Fc-HRS(2-60), is unaffected. Although SEMA3C and mouse SEMA3F do bind to NRP2, the presence of either of these proteins also do not affect NRP2 binding to Fc-HRS(2-60) under the conditions tested. These results suggest that the Fc-HRS(2-60) binding site of NRP2 overlaps with the VEGF binding site, but not with the SEMA binding site of NRP2.

TABLE E20

| | Binding to NRP2 | Competes with Fc-HRS(2-60) for |
|---|---|---|
| Ligand | observed by SPR | NRP2 binding |
| VEGF-C | Yes | Yes |
| VEGF-A₁₆₅ | Yes | Yes |
| VEGF-A₁₄₅ | No | No |
| VEGF-A₁₂₁ | No | No |
| PlGf-2/Heparin | Not tested | Yes |
| SEMA3C | Yes | No |
| Mouse SEMA3F | Yes | No |

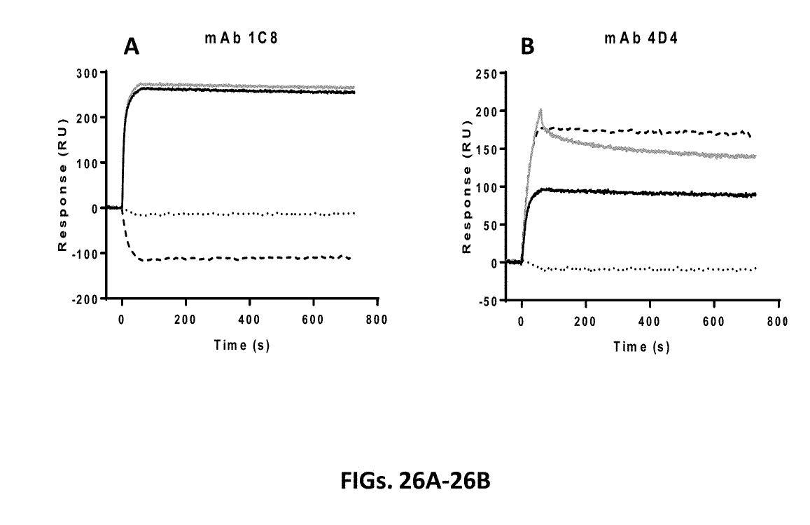

In another series of experiments, different monoclonal antibodies which recognize Fc-HRS(2-60) were immobilized on SPR chips. In FIGS. 26A-26B, the mAb clones 1C8 and 4D4 were immobilized on an SPR chip and then a mixture of Fc-HRS(2-60) and NRP2 which had been pre-incubated together was injected over the mAb surfaces.

Based on the resulting pattern of signal intensities it can be concluded that the monoclonal antibody clone 1C8 likely binds to Fc-HRS(2-60) at an epitope involved in NRP2 binding, because no larger complex binding is detected when the complex is passed over the detection surface. The lack of additional binding under these conditions suggests that the 1C8 antibody is capable of displacing Nrp-2 from the Fc-HRS(2-60):Nrp-2 complex.

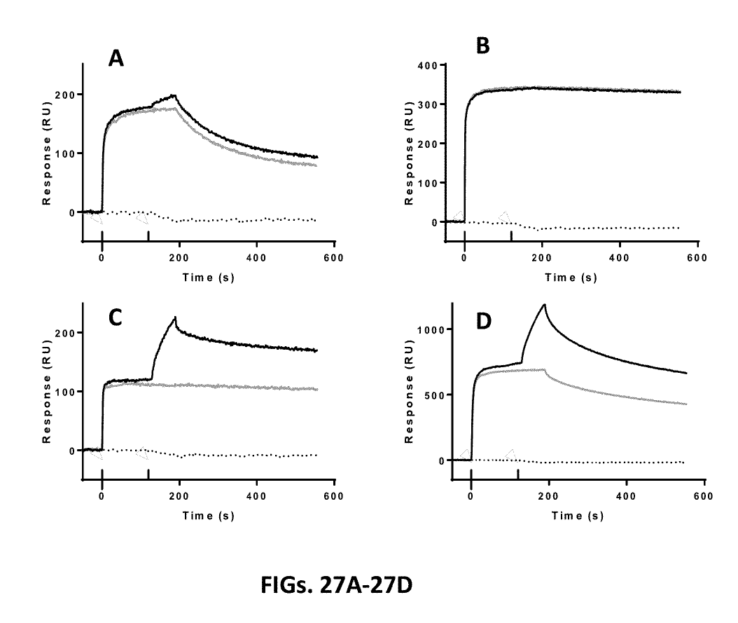

In contrast, when the monoclonal antibody clone 4D4 was attached to the detection surface, a significantly larger signal intensity was observed, suggesting that it was able to bind to the Fc-HRS(2-60) moiety, without displacing Nrp-2, from the Fc-HRS(2-60):Nrp-2. This indicates that NRP2 is able to bind Fc-HRS(2-60) in the presence of the 4D4 mAb and that they bind to non-overlapping regions of Fc-HRS(2-60). Additionally, co-injection experiments were carried out in which sequential analyte injections of Fc-HRS(2-60) followed by NRP2 were performed (FIGS. 27A-27D). In these experiments, Fc-HRS(2-60) that was bound to antibody clone ATYR4D4 or monoclonal antibody clone ATYR13E9 were able to further bind NRP2.

Fc-HRS(2-60) that was bound to monoclonal antibody clone ATYR12H6 showed only slight binding of NRP2, while Fc-HRS(2-60) bound to antibody clone ATYR1C8 showed no binding to NRP2. These data together indicate that antibody clone ATYR1C8 binding is able to block NRP2 binding to Fc-HRS(2-60), while antibody clone ATYR12H6 binding is able to partially block NRP2 binding, and antibody clones ATYR4D4 and ATYR13E9 are not able to block NRP2 binding to HRS.

### Example 21

### Confirmation of Binding to NRP2 Expressed in HEK293 Cells

To directly confirm direct binding of HRS to cells expressing recombinant Neuropilin 2a or 2b, Fc-HRS(2-60) was added to HEK293 cells which had been transfected with expression vectors encoding for either Neuropilin 2a or 2b, or as their respective fusion proteins with GFP, and detected via the use of fluorescently labelled anti-Fc-PE as described in the Materials and Methods.

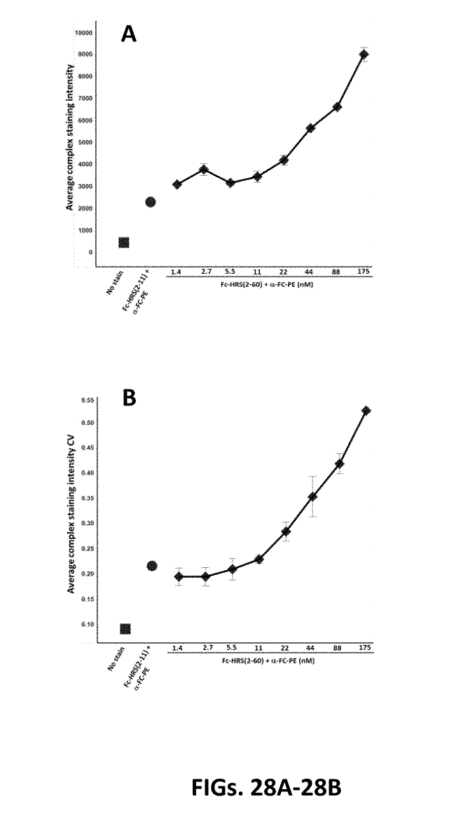

As shown in FIGS. 28A-28B, dose-dependent binding of Fc-HRS (2-60) to cell-expressed NRP2a was readily detectable under these conditions.

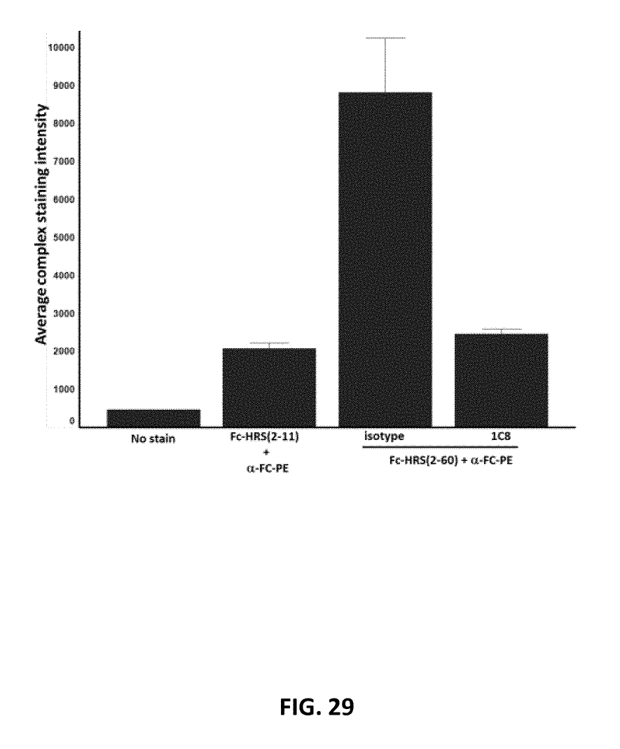

FIG. 29 shows that pre-incubation of Fc-HRS(2-60) with the blocking antibody clone 1C8, resulted in almost complete abolition of binding, demonstrating that the binding is specific for the epitope recognized by the anti-HRS antibody. Binding specificity was further confirmed through the use of the deleted control protein, Fc-HRS(2-11), which also showed negligible specific binding.

To determine the ability of anti-HRS antibodies to block binding of Fc-HRS (2-60) to NRP2, HEK293 cells were stably transfected with NRP2 and binding of biotinylated Fc-HRS (2-60) in the presence or absence of antibodies monitored by flow cytometry as described in the Materials and Methods.

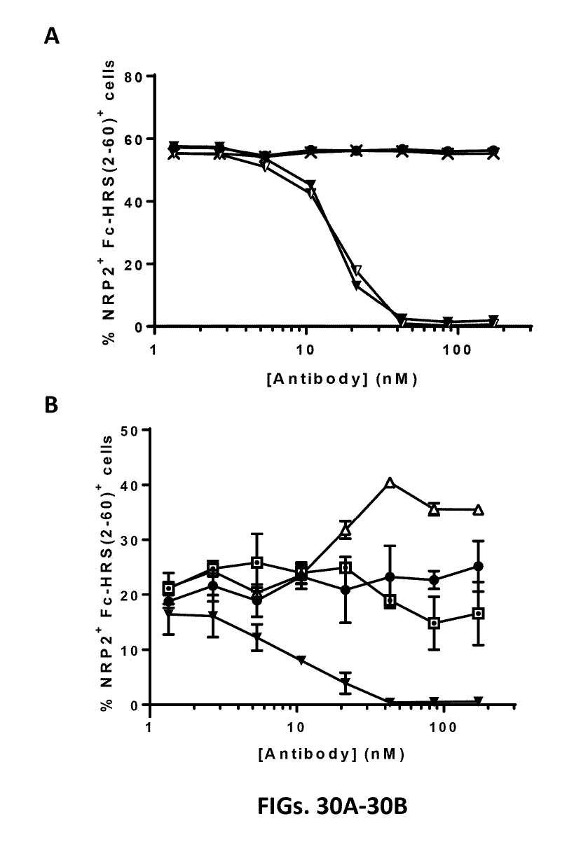

FIG. 30A-30B shows that antibodies from the KL31 series blocked binding of Fc-HRS to NRP2 in a concentration-dependent manner, whereas other antibodies tested did not demonstrate significant blocking characteristics in this assay.

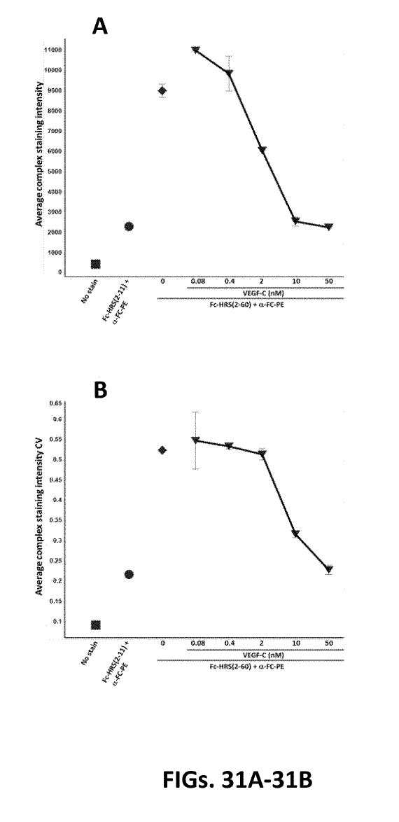

Functional interactions with other Neuropilin 2 interacting proteins was demonstrated via direct competition of Fc-HRS(2-60) by pre-incubation of cells expression NRP2 with VEGF-C (FIGS. 31A-31B).

These results confirm, and extend the Retrogenix screens and suggests that the interaction of HRS proteins such as wild type HRS, and HRS polypeptides comprising the N-terminal region play important, biologically relevant roles by binding to NRP2, and by interacting with its other natural ligands including VEGF-C.

### Example 22

### Circulating Levels of Soluble Neuropilin 2 (NRP2) in Human Serum and Plasma

Serum & plasma samples from normal healthy volunteers (n=72) were tested for circulating levels of soluble NRP2. NRP2 levels were quantified with an internally developed human NRP2 ELISA (as described in the Materials and Methods).

Summary of Results.

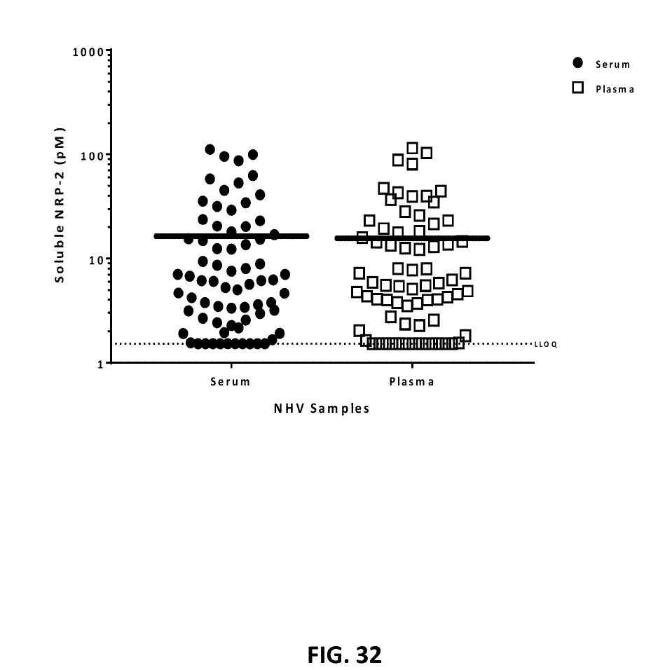

Analysis of circulating NRP2 in both serum and plasma revealed complimentary results in both matrices. Serums levels of NRP2 averaged 16.3 pM while mean plasma levels were 15.6 pM. Quantification revealed that 86% of the serum samples and 83% of the plasma samples were detectable and above the lower limit of quantitation for this assay (1.5 pM) (Table E21 and FIG. 32).

TABLE E21

| | Serum | Plasma |
|---|---|---|
| # of samples | 72 | 72 |
| Mean +/− SD (pM) | 16.3 +/− 24.3 | 15.6 +/− 23.3 |
| Median (pM) | 6.1 | 5.5 |
| Range (pM) | ≤1.5-111.6 | ≤1.5-115.3 |

### Example 23

### Comparison of Circulating HRS & NRP2 Levels

Circulating serum HRS levels from 72 normal healthy donors were rank ordered from lowest to highest levels. Matching serum NRP2 levels from the exact same donors were overlaid on the same axes.

Summary of Results.

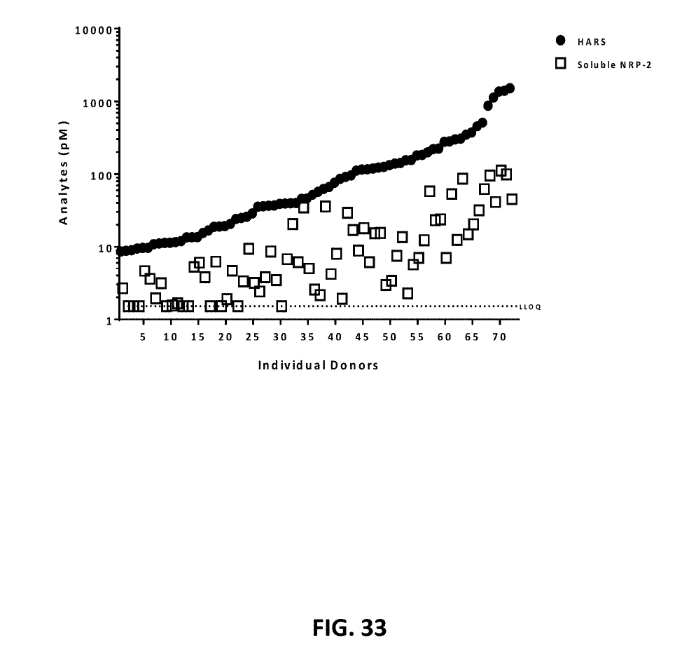

Human HRS levels from normal healthy donors spans nearly two logs (˜10 pM-1000 pM) in concentration. Similarly, soluble NRP2 levels also demonstrate a large distribution in circulating levels (˜1 pM-100 pM). Comparison of serum samples from normal healthy volunteers revealed a trend whereby people with low circulating HRS levels also have lower soluble NRP2 levels and conversely those individuals with higher HRS levels demonstrate higher circulating soluble NRP2 levels (see FIG. 33).

### Example 24

### N-Terminal HRS Assay Interference

Serum samples from normal healthy volunteers were assayed in two separate ELISAs to detect circulating levels of HRS. An assay designed to detect the full length version of HARS (HARS_FL) utilized an N-terminal capture antibody and a C-terminal detection antibody. The second assay was designed to exclusively detect the N-terminal portion of HRS (HARS_NT) with both capture and detection antibodies directed to the N-terminus. Accordingly, the FL-terminal assay, is unable to detect N-terminally truncated fragments of HRS, lacking the C-terminal epitope recognized by the C-terminal detection antibody. Conversely the N-terminal assay is susceptible to interference via the binding of other factors to the N-terminal domain of HRS, which compete with antibody binding.

Summary of Results.

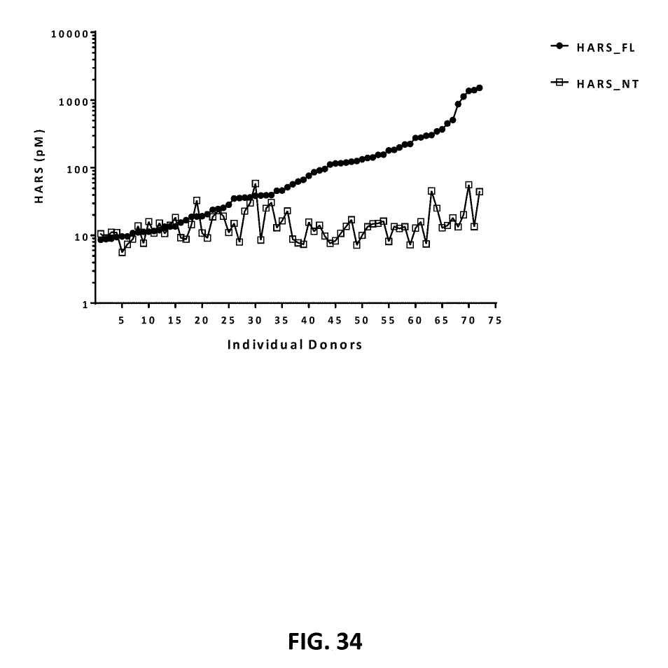

Individual healthy donor serum was assayed for HRS levels using both the full length and N-terminal ELISA formats. Samples with low levels of HRS detected via the full length ELISA HRS levels tended to correlate well with the N-terminal ELISA results (FIG. 34). However, in selected donors with relatively high levels of HRS detected via the FL-ELISA, it was also observed that the HRS levels detected via the N-terminal ELISA no longer showed a close correlation, but were in certain subjects significantly lower. Without being bound by any one specific explanation, it is believed that the significantly lower apparent HRS levels in the N-terminal assay is caused by the existence of an interfering substance which binds to the N-terminal domain of HRS, thereby blocking its detection in the N-terminal ELISA assay.

### Example 25

### Correlation of Hrs N-Terminal Interference and Soluble NRP2

To further examine the relationship between HRS N-terminal assay interference and soluble NRP2 levels, circulating HRS and NRP-2 levels were analyzed in normal healthy volunteer serum samples. The difference in observed HRS levels between the full length ELISA and the N-terminal ELISA was calculated for each of the 72 healthy serum donor samples (N-terminal Interference Units). These same samples were additionally tested for circulating human NRP2 levels.

Summary of Results.

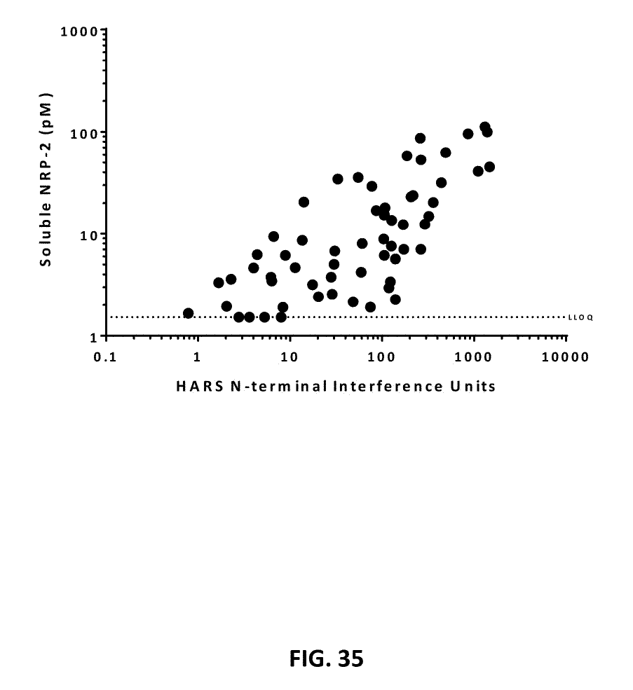

The interference observed between the two HRS assay formats was termed HARS N-terminal Interference Units (HARS_FL minus HARS_NT) and was plotted versus soluble NRP2 levels (FIG. 35). The resulting graph shows a clear trend for increased N-terminal interference and increased soluble NRP2 levels suggesting a potential role for soluble NRP2 to interfere with the detection of the N-terminus of HRS.

### Example 26

### Detection of HRS:NRP2 Soluble Complex in Normal Serum

In an attempt to observe an endogenous circulating HRS: NRP2 soluble complex in serum, several novel ELISA formats were utilized to capture this interaction. Normal healthy human serum was isolated from internal sources (#21949, #32565, #22447, #24098, #23024) or through commercial vendors (Sigma, CELLect). These healthy serum samples were analyzed for levels of N-terminal interference (data not shown) and categorized as either low N-terminal interference or high N-terminal interference and parsed accordingly. These 7 serum samples were assayed in multiple formats of a HRS:NRP-2 complex ELISA. These assays consisted of a capture antibody directed against HRS N-terminus (HARS_NT), HARS C-terminus (HARS_CT), or NRP2. The detection antibody in these assays was directed against the alternate protein in the complex (e.g., HRS detection antibody with a NRP2 capture antibody, and NRP2 detection antibody with a HRS capture antibody).

Summary of Results.

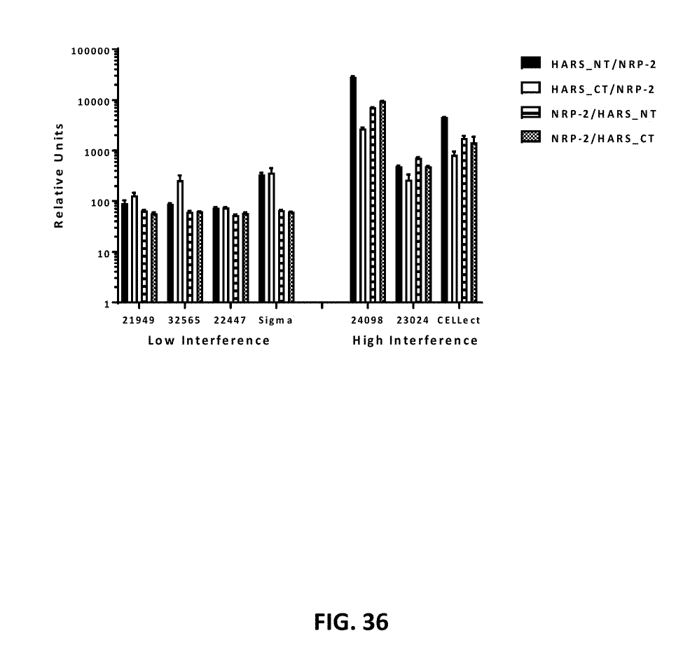

HRS:NRP2 complex ELISAs were tested with normal serum samples that had been previously identified as either having low or high N-terminal interference. All samples with low N-terminal HRS interference showed low signals in all formats of the HRS:NRP2 complex ELISAs (FIG. 36, left bar graphs). In contrast, serum samples identified as containing high N-terminal assay interference all showed elevated signals in HRS and NRP-2 complex ELISAs (FIG. 36, right bar graphs). These results were observed with multiple antibody pairings to both terminal ends of HRS, suggesting that the result is not the result of unanticipated antibody cross reactivities between NRP2 and HRS.

### Example 27

### Confirmation of a HRS & NRP2 Soluble Complex in Normal Serum

To confirm the relationship between HRS N-terminal interference and the detection of an endogenous soluble HRS:NRP2 complex, the antibody reagents utilized to originally characterize the N-terminal interference observed in human serum were tested side by side in the HRS:NRP2 complex ELISA. Healthy normal serum samples from persons identified as low or high interference (as described above) were tested in a HRS:NRP2 complex ELISA consisting of an NRP2 capture antibody followed by detection with either a non-interfering HRS N-terminal antibody (HARS_NT) or an N-terminal HRS antibody that blocks the interaction (HRS blocking antibody).

Summary of Results.

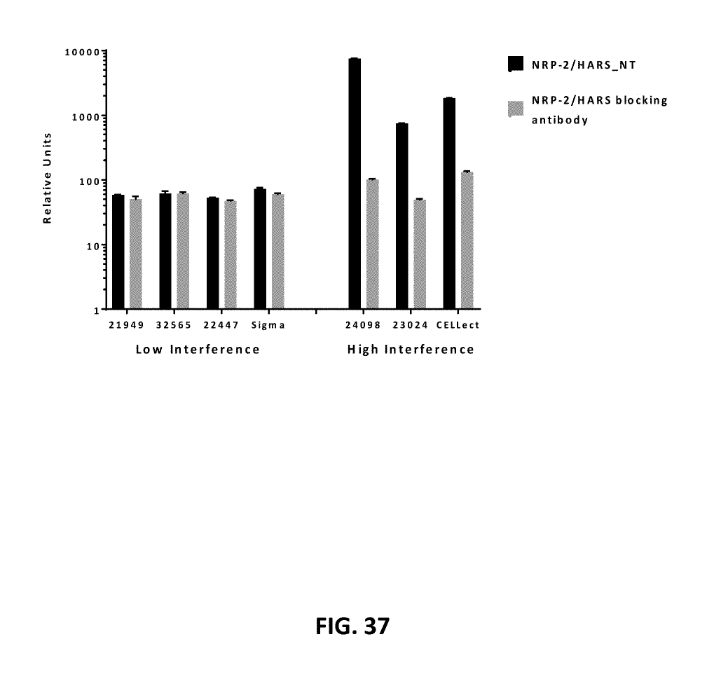

The results of the HRS:NRP2 complex ELISA show an increased signal between low and high interference samples when capturing soluble NRP2 and detecting with the HARS_NT antibody. However, when these same sample are tested in an assay format where the detection antibody against HRS is directed against the site where NRP2 is believed to bind, then the signal in this complex ELISA returns to the same levels as observed in samples without assay interference (FIG. 37). The results suggest that this blocking antibody is directed against the putative NRP2 binding site on the N-terminus of HRS.

### Example 28

### HRS Levels in Healthy Individuals and Cancer Subjects

To examine the relationship between HRS levels and cancer progression, HRS levels were analyzed in plasma using the full length HRS ELISA in 148 normal volunteer samples, 215 samples from patients with solid tumors, and 100 samples from patients with hematologic tumors obtained from Conversant Bio.

Summary of Results.

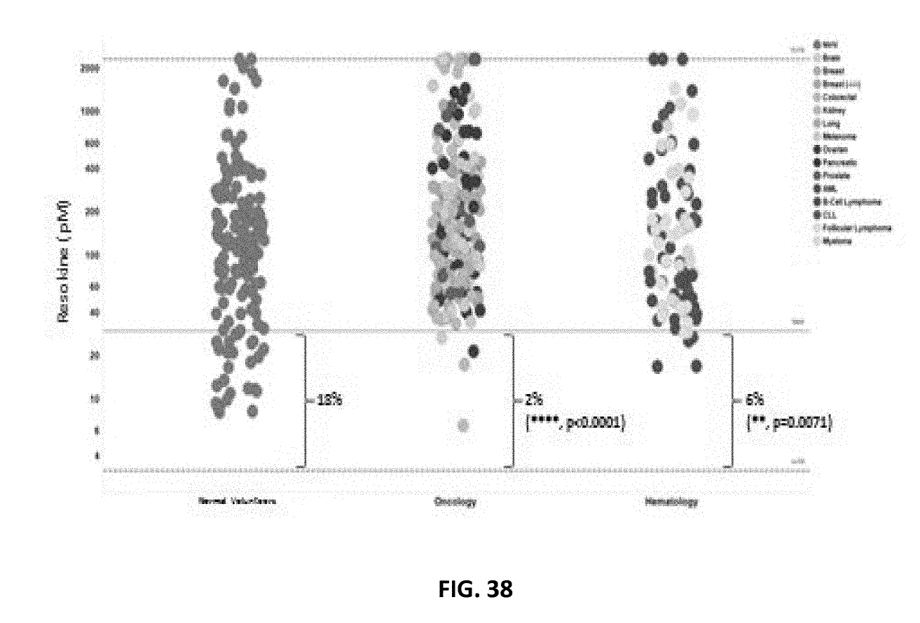

The results show an elevation of HRS baseline levels in all (15/15) cancer types tested compared to normal healthy controls (FIG. 38). In healthy volunteer samples (n=148) HRS levels ranged from ˜8 pM to ˜2,000 pM with 18% of the individuals possessing a level below 30 pM. In contrast, HRS levels measured across patients with all tumor types tested in an initial sample set (n=215, ranged from ˜20 pM to >2,333 pM (above the upper limit of quantification) with less than 5% of the patients possessing low levels defined as <30 pM; (P<0.0001).

Without being bound by any one particular theory of operation, this data is consistent with the hypothesis that tumors secrete HRS, which acts at least in part as an immuno-shielding protein to avoid detection by the immune system. Given the immune modulating activity of HRS polypeptides, the measurement of extracellular HRS derived proteins offers a new liquid biopsy biomarker for tracking immune cell activity in cancer patients. The data provided herein also supports the concept that HRS polypeptides can form the foundation for new therapeutic approaches for cancer diagnosis and prognosis, and that anti-HRS antibodies have the potential for use in treatment and prevention of cancer, including in patients or populations with increased levels of HRS polypeptides.

### Example 29

### Evaluation of Human Jo-1 Antibodies on B16-F10 Mouse Melanoma in C57BL/6 Mice

This study was designed to investigate the in vivo anticancer potential of fully human Jo-1 antibodies cloned from individuals identified as Jo-1 positive (as described in the Materials and Methods Section) in a syngeneic mouse model using B16-F10 cells (melanoma cancer model) prepared as described previously. The test antibody dosing regimen was initiated one day before cell injection and animal weights and tumor measurements were recorded three times a week until study termination.

Treatment Regimen.

Ninety (90) C57BL/6 mice (Jax, female, 5-6 wks old) were used in this study. For brevity, this example focuses on 4 treatment groups. The animals were assigned to study groups of 10 mice randomly, and housed as described in the Materials and Methods. The antibody dosing regimen is shown in Table E22 below; in brief animals received intraperitoneal injections of 10 mg/kg of each of the control IgG antibodies, positive control αPD-L1 antibody, and anti-HARS antibodies 13E9, or fully human Jo-1 antibody AB04, administered according to the protocol below (Table E22); starting one day before cancer cell implantation and then on day 6 and 13 post-cell implantation.

TABLE E22
GROUP TREATMENTS

| | | | | Route of | |
|---|---|---|---|---|---|
| | | Test | Dose | Administration | |
| Group | #Mice | Materials | (mg/kg) | (ROA) | Frequency |
| 1 | 10 | Control | 20 | IP | days −1, 6, 13 |
| | | IgG | | | |
| 2 | 10 | αPD-L1 + | 10 | IP | days −1, 6, 13 |
| | | Control | 10 | | |
| | | IgG | | | |
| 3 | 10 | 13E9 + | 10 | IP | days −1, 6, 13 |
| | | Control | 10 | | |
| | | IgG | | | |
| 4 | 10 | AB04 + | 10 | IP | days −1, 6, 13 |
| | | Control | 10 | | |
| | | IgG | | | |

Summary of Results.

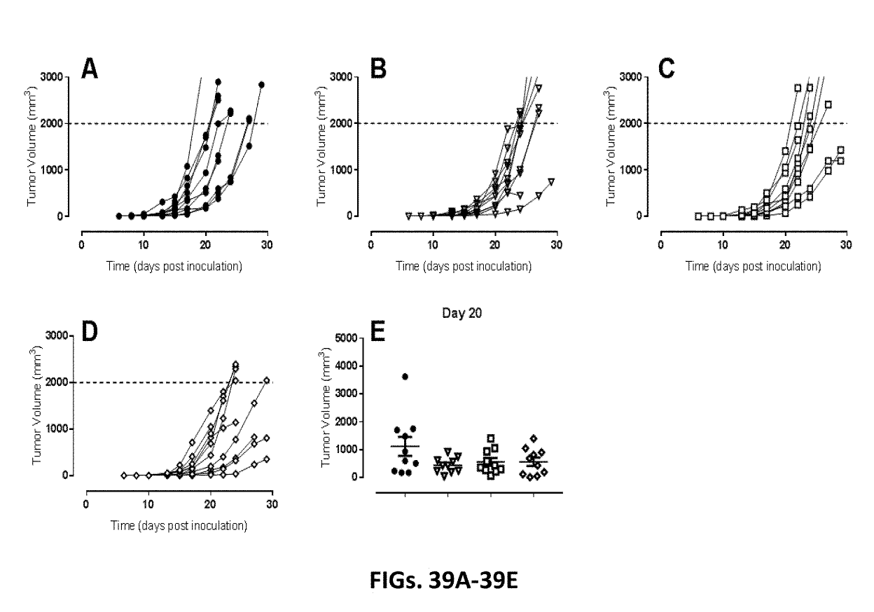

Animals bearing B16-F10 tumors and treated with 3 doses of 13E9 or the human Jo-1 antibody AB04 showed reduced tumor growth (FIG. 39A-39F). Animals bearing B16-F10 tumors and treated with 3 doses of anti-mouse PD-L1 showed similarly reduced tumor growth (compare tumor volumes on Day 20, the last day all animals were on study). There was no evidence of toxicity from animal body weight measurements and observations during the study (data not shown). These results demonstrate that recombinant Jo-1 antibodies, (e.g. anti-HARS antibodies) have clear potential for a prophylactic and potentially therapeutic impact on cancer growth in this model system.

### Example 30

### Human Jo-1 Antibodies Inhibit Tumor Growth and Enhance Tumor Growth Inhibition in Combination with PD-L1 Pathway Blockade in the CT26 Tumor Model

This study aims to investigate the in vivo anti-cancer potential of human Jo-1 antibodies (e.g. naturally occurring anti-HARS antibodies) alone or in combination with anti-PD-L1 antibodies in a syngeneic mouse model using CT26 cells (colon cancer model) prepared as described in Materials and Methods. In this study, the dosing regimen (Table E23) was initiated one day before cell injection (i.e., prophylactically). The animal weights and tumor measurements were recorded three times a week.

Treatment Regimen.

One hundred (100) Balb/c mice (Envigo, female, 5-6 wks old) were used in this study. The following focuses groups of 10 mice which were randomly assigned to 8 treatments. The CT26 cell line was expanded for injection as described in the Materials and Methods. The dosing regimen is shown in Table E23 below; In brief animals received injections of 10 mg/kg of each control IgG, positive control antibodies, and anti-HARS antibodies as described in the Materials and Methods, which were administered to mice intraperitoneally starting one day before cancer cell implantation and on days 6 and 13 post implantation) according to the protocol below.

TABLE E23
GROUP TREATMENTS

| | | | Dose | | |
|---|---|---|---|---|---|
| Group | #Mice | Materials | (mg/kg) | ROA | Frequency |
| 1 | 10 | hIgG1 + | 10 | IP | Days −1, 6, 13 |
| | | rIgG2b | 10 | | |
| 2 | 10 | hIgG1 + | 10 | IP | Days −1, 6, 13 |
| | | αmPD-L1 | 10 | | |
| 3 | 10 | 13E9 + | 10 | IP | Days −1, 6, 13 |
| | | rIgG2b | 10 | | |
| 4 | 10 | AB13 + | 10 | IP | Days −1, 6, 13 |
| | | rIgG2b | 10 | | |
| 5 | 10 | 13E9 + | 10 | IP | Days −1, 6, 13 |
| | | αmPD-L1 | 10 | | |
| 6 | 10 | AB13 + | 10 | IP | Days −1, 6, 13 |
| | | αmPD-L1 | 10 | | |
| 7 | 10 | No treatment | | | |
| 8 | 10 | No tumor, no | | | |
| | | treatment | | | |

Summary of Results.

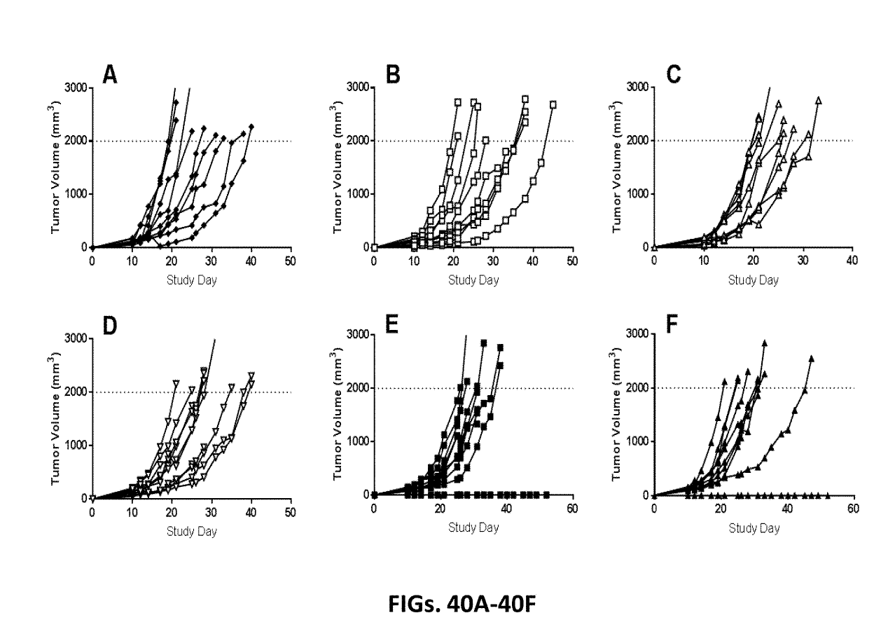

There was no evidence of toxicity from weight measurements and observations during the study (data not shown). Animals bearing CT26 tumors treated with 3 doses of naturally occurring Jo-1 antibodies, (e.g. anti-HARS antibodies) together with an anti-PD-L1 antibody showed more effective inhibition of tumor growth compared to tumor-bearing untreated controls beginning at study day 19 (FIGS. 40A-40H). Furthermore, both HARS binding antibodies had a least 1 animal that never grew a tumor with prophylactic treatment.

These results demonstrate that recombinant Jo-1 antibodies, (e.g. anti-HARS antibodies), alone and in combination with anti-PD-L1 blockade have clear potential for a prophylactic and potentially therapeutic impact on cancer growth in this colon cancer model system as well as the potential to synergize with other anti-cancer approaches.

1. A therapeutic composition, comprising at least one antibody or antigen-binding fragment there that specifically binds to a human histidyl-tRNA synthetase (HRS) polypeptide (an anti-HRS antibody) and comprises a heavy chain variable region (V_{H}) sequence that comprises complementary determining region V_{H}CDR1, V_{H}CDR2, and V_{H}CDR3 sequences, and a light chain variable region (V_{L}) sequence that comprises complementary determining region V_{L}CDR1, V_{L}CDR2, and V_{L}CDR3 sequences, wherein:
the V_{H}CDR1, V_{H}CDR2, and V_{H}CDR3 sequences comprise the consensus sequences of SEQ ID NOs:396, 397, and 398 (as defined in Table A3), respectively, and the V_{L}CDR1, V_{L}CDR2, and V_{L}CDR3 sequences comprise the consensus sequences SEQ ID NOs: 399, 400, and 401 (as defined in Table A3), respectively, including variants thereof;
the V_{H}CDR1, V_{H}CDR2, and V_{H}CDR3 sequences comprise the consensus sequences of SEQ ID NOs: 402, 403, and 404 (as defined in Table A3), respectively, and the V_{L}CDR1, V_{L}CDR2, and V_{L}CDR3 sequences comprise the consensus sequences of SEQ ID NOs: 405, 406, and 407 (as defined in Table A3), respectively, including variants thereof;
the V_{H}CDR1, V_{H}CDR2, and V_{H}CDR3 sequences comprise the consensus sequences of SEQ ID NOs: 408, 409, and 410 (as defined in Table A3), respectively, and the V_{L}CDR1, V_{L}CDR2, and V_{L}CDR3 sequences comprise the consensus sequences of SEQ ID NOs: 411, 412, and 413 (as defined in Table A3), respectively, including variants thereof;
the V_{H}CDR1, V_{H}CDR2, and V_{H}CDR3 sequences comprise SEQ ID NOs: 12, 13, and 14, respectively, and the V_{L}CDR1, V_{L}CDR2, and V_{L}CDR3 sequences comprise SEQ ID NOs: 15, 16, and 17, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 18, 19, and 20, respectively, and the V_{L}CDR1, V_{L}CDR2, and V_{L}CDR3 sequences comprise SEQ ID NOS: 21, 22, and 23, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 24, 25, and 26, respectively, and the V_{L}CDR1, V_{L}CDR2, and V_{L}CDR3 sequences comprise SEQ ID NOs: 27, 28, and 29, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 36, 37, and 38, respectively, and the V_{L}CDR1, V_{L}CDR2, and V_{L}CDR3 sequences comprise SEQ ID NOs: 39, 40, and 41, respectively, including variants thereof;
the V_{H}CDR1, V_{H}CDR2, and V_{H}CDR3 sequences comprise SEQ ID NOs: 42, 43, and 44, respectively, and the V_{L}CDR1, V_{L}CDR2, and V_{L}CDR3 sequences comprise SEQ ID NOs: 45, 46, and 47, respectively, including variants thereof;
the V_{H}CDR1, V_{H}CDR2, and V_{H}CDR3 sequences comprise SEQ ID NOs: 48, 49, and 50, respectively, and the V_{L}CDR1, V_{L}CDR2, and V_{L}CDR3 sequences comprise SEQ ID NOs: 51, 52, and 53, respectively, including variants thereof;
the V_{H}CDR1, V_{H}CDR2, and V_{H}CDR3 sequences comprise SEQ ID NOs: 54, 55, and 56, respectively, and the V_{L}CDR1, V_{L}CDR2, and V_{L}CDR3 sequences comprise SEQ ID NOs: 57, 58, and 59, respectively, including variants thereof;
the V_{H}CDR1, V_{H}CDR2, and V_{H}CDR3 sequences comprise SEQ ID NOs: 60, 61, and 62, respectively, and the V_{L}CDR1, V_{L}CDR2, and V_{L}CDR3 sequences comprise SEQ ID NOS: 63, 64, and 65, respectively, including variants thereof;
the V_{H}CDR1, V_{H}CDR2, and V_{H}CDR3 sequences comprise SEQ ID NOs: 66, 67, and 68, respectively, and the V_{L}CDR1, V_{L}CDR2, and V_{L}CDR3 sequences comprise SEQ ID NOs: 69, 70, and 71, respectively, including variants thereof;
the V_{H}CDR1, V_{H}CDR2, and V_{H}CDR3 sequences comprise SEQ ID NOs: 72, 73, and 74, respectively, and the V_{L}CDR1, V_{L}CDR2, and V_{L}CDR3 sequences comprise SEQ ID NOs: 75, 76, and 77, respectively, including variants thereof;
the V_{H}CDR1, V_{H}CDR2, and V_{H}CDR3 sequences comprise SEQ ID NOs: 78, 79, and 80, respectively, and the V_{L}CDR1, V_{L}CDR2, and V_{L}CDR3 sequences comprise SEQ ID NOS: 81, 82, and 83, respectively, including variants thereof;
the V_{H}CDR1, V_{H}CDR2, and V_{H}CDR3 sequences comprise SEQ ID NOs: 84, 85, and 86, respectively, and the V_{L}CDR1, V_{L}CDR2, and V_{L}CDR3 sequences comprise SEQ ID NOs: 87, 88, and 89, respectively, including variants thereof;
the V_{H}CDR1, V_{H}CDR2, and V_{H}CDR3 sequences comprise SEQ ID NOs: 90, 91, and 92, respectively, and the V_{L}CDR1, V_{L}CDR2, and V_{L}CDR3 sequences comprise SEQ ID NOs: 93, 94, and 95, respectively, including variants thereof;
the V_{H}CDR1, V_{H}CDR2, and V_{H}CDR3 sequences comprise SEQ ID NOs: 96, 97, and 98, respectively, and the V_{L}CDR1, V_{L}CDR2, and V_{L}CDR3 sequences comprise SEQ ID NOs: 99, 100, and 101, respectively, including variants thereof;
the V_{H}CDR1, V_{H}CDR2, and V_{H}CDR3 sequences comprise SEQ ID NOs: 102, 103, and 104, respectively, and the V_{L}CDR1, V_{L}CDR2, and V_{L}CDR3 sequences comprise SEQ ID NOS: 105, 106, and 107, respectively, including variants thereof;
the V_{H}CDR1, V_{H}CDR2, and V_{H}CDR3 sequences comprise SEQ ID NOs: 108, 109, and 110, respectively, and the V_{L}CDR1, V_{L}CDR2, and V_{L}CDR3 sequences comprise SEQ ID NOs: 111, 112, and 113, respectively, including variants thereof;
the V_{H}CDR1, V_{H}CDR2, and V_{H}CDR3 sequences comprise SEQ ID NOs: 114, 115, and 116, respectively, and the V_{L}CDR1, V_{L}CDR2, and V_{L}CDR3 sequences comprise SEQ ID NOs: 117, 118, and 119, respectively, including variants thereof;
the V_{H}CDR1, V_{H}CDR2, and V_{H}CDR3 sequences comprise SEQ ID NOs: 120, 121, and 122, respectively, and the V_{L}CDR1, V_{L}CDR2, and V_{L}CDR3 sequences comprise SEQ ID NOs: 123, 124, and 125, respectively, including variants thereof;
the V_{H}CDR1, V_{H}CDR2, and V_{H}CDR3 sequences comprise SEQ ID NOs: 126, 127, and 128, respectively, and the V_{L}CDR1, V_{L}CDR2, and V_{L}CDR3 sequences comprise SEQ ID NOs: 129, 130, and 131, respectively, including variants thereof;
the V_{H}CDR1, V_{H}CDR2, and V_{H}CDR3 sequences comprise SEQ ID NOs: 132, 133, and 134, respectively, and the V_{L}CDR1, V_{L}CDR2, and V_{L}CDR3 sequences comprise SEQ ID NOs: 135, 136, and 137, respectively, including variants thereof;
the V_{H}CDR1, V_{H}CDR2, and V_{H}CDR3 sequences comprise SEQ ID NOs: 138, 139, and 140, respectively, and the V_{L}CDR1, V_{L}CDR2, and V_{L}CDR3 sequences comprise SEQ ID NOs: 141, 142, and 143, respectively, including variants thereof;
the V_{H}CDR1, V_{H}CDR2, and V_{H}CDR3 sequences comprise SEQ ID NOs: 144, 145, and 146, respectively, and the V_{L}CDR1, V_{L}CDR2, and V_{L}CDR3 sequences comprise SEQ ID NOs: 147, 148, and 149, respectively, including variants thereof;
the V_{H}CDR1, V_{H}CDR2, and V_{H}CDR3 sequences comprise SEQ ID NOs: 150, 151, and 152, respectively, and the V_{L}CDR1, V_{L}CDR2, and V_{L}CDR3 sequences comprise SEQ ID NOs: 153, 154, and 155, respectively, including variants thereof;
the V_{H}CDR1, V_{H}CDR2, and V_{H}CDR3 sequences comprise SEQ ID NOs: 156, 157, and 158, respectively, and the V_{L}CDR1, V_{L}CDR2, and V_{L}CDR3 sequences comprise SEQ ID NOS: 159, 160, and 161, respectively, including variants thereof;
the V_{H}CDR1, V_{H}CDR2, and V_{H}CDR3 sequences comprise SEQ ID NOs: 162, 163, and 164, respectively, and the V_{L}CDR1, V_{L}CDR2, and V_{L}CDR3 sequences comprise SEQ ID NOs: 165, 166, and 167, respectively, including variants thereof;
the V_{H}CDR1, V_{H}CDR2, and V_{H}CDR3 sequences comprise SEQ ID NOs: 168, 169, and 170, respectively, and the V_{L}CDR1, V_{L}CDR2, and V_{L}CDR3 sequences comprise SEQ ID NOs: 171, 172, and 173, respectively, including variants thereof;
the V_{H}CDR1, V_{H}CDR2, and V_{H}CDR3 sequences comprise SEQ ID NOs: 174, 175, and 176, respectively, and the V_{L}CDR1, V_{L}CDR2, and V_{L}CDR3 sequences comprise SEQ ID NOs: 177, 178, and 179, respectively, including variants thereof;
the V_{H}CDR1, V_{H}CDR2, and V_{H}CDR3 sequences comprise SEQ ID NOs: 180, 181, and 182, respectively, and the V_{L}CDR1, V_{L}CDR2, and V_{L}CDR3 sequences comprise SEQ ID NOs: 183, 184, and 185, respectively, including variants thereof;
the V_{H}CDR1, V_{H}CDR2, and V_{H}CDR3 sequences comprise SEQ ID NOs: 186, 187, and 188, respectively, and the V_{L}CDR1, V_{L}CDR2, and V_{L}CDR3 sequences comprise SEQ ID NOs: 189, 190, and 191, respectively, including variants thereof;
the V_{H}CDR1, V_{H}CDR2, and V_{H}CDR3 sequences comprise SEQ ID NOs: 192, 193, and 194, respectively, and the V_{L}CDR1, V_{L}CDR2, and V_{L}CDR3 sequences comprise SEQ ID NOs: 195, 196, and 197, respectively, including variants thereof;
the V_{H}CDR1, V_{H}CDR2, and V_{H}CDR3 sequences comprise SEQ ID NOs: 198, 199, and 200, respectively, and the V_{L}CDR1, V_{L}CDR2, and V_{L}CDR3 sequences comprise SEQ ID NOs: 201, 202, and 203, respectively, including variants thereof;
the V_{H}CDR1, V_{H}CDR2, and V_{H}CDR3 sequences comprise SEQ ID NOs: 204, 205, and 206, respectively, and the V_{L}CDR1, V_{L}CDR2, and V_{L}CDR3 sequences comprise SEQ ID NOs: 207, 208, and 209, respectively, including variants thereof;
the V_{H}CDR1, V_{H}CDR2, and V_{H}CDR3 sequences comprise SEQ ID NOs: 210, 211, and 212, respectively, and the V_{L}CDR1, V_{L}CDR2, and V_{L}CDR3 sequences comprise SEQ ID NOS: 213, 214, and 215, respectively, including variants thereof;
the V_{H}CDR1, V_{H}CDR2, and V_{H}CDR3 sequences comprise SEQ ID NOs: 216, 217, and 218, respectively, and the V_{L}CDR1, V_{L}CDR2, and V_{L}CDR3 sequences comprise SEQ ID NOs: 219, 220, and 221, respectively, including variants thereof;
the V_{H}CDR1, V_{H}CDR2, and V_{H}CDR3 sequences comprise SEQ ID NOs: 222, 223, and 224, respectively, and the V_{L}CDR1, V_{L}CDR2, and V_{L}CDR3 sequences comprise SEQ ID NOs: 225, 226, and 227, respectively, including variants thereof;
the V_{H}CDR1, V_{H}CDR2, and V_{H}CDR3 sequences comprise SEQ ID NOs: 228, 229, and 230, respectively, and the V_{L}CDR1, V_{L}CDR2, and V_{L}CDR3 sequences comprise SEQ ID NOs: 231, 232, and 233, respectively, including variants thereof;
the V_{H}CDR1, V_{H}CDR2, and V_{H}CDR3 sequences comprise SEQ ID NOs: 234, 235, and 236, respectively, and the V_{L}CDR1, V_{L}CDR2, and V_{L}CDR3 sequences comprise SEQ ID NOs: 237, 238, and 239, respectively, including variants thereof;
the V_{H}CDR1, V_{H}CDR2, and V_{H}CDR3 sequences comprise SEQ ID NOs: 240, 241, and 242, respectively, and the V_{L}CDR1, V_{L}CDR2, and V_{L}CDR3 sequences comprise SEQ ID NOs: 243, 244, and 245, respectively, including variants thereof;
the V_{H}CDR1, V_{H}CDR2, and V_{H}CDR3 sequences comprise SEQ ID NOs: 246, 247, and 248, respectively, and the V_{L}CDR1, V_{L}CDR2, and V_{L}CDR3 sequences comprise SEQ ID NOs: 249, 250, and 251, respectively, including variants thereof;
the V_{H}CDR1, V_{H}CDR2, and V_{H}CDR3 sequences comprise SEQ ID NOs: 252, 253, and 254, respectively, and the V_{L}CDR1, V_{L}CDR2, and V_{L}CDR3 sequences comprise SEQ ID NOs: 255, 256, and 257, respectively, including variants thereof;
the V_{H}CDR1, V_{H}CDR2, and V_{H}CDR3 sequences comprise SEQ ID NOs: 258, 259, and 260, respectively, and the V_{L}CDR1, V_{L}CDR2, and V_{L}CDR3 sequences comprise SEQ ID NOs: 261, 262, and 263, respectively, including variants thereof;
the V_{H}CDR1, V_{H}CDR2, and V_{H}CDR3 sequences comprise SEQ ID NOs: 264, 265, and 266, respectively, and the V_{L}CDR1, V_{L}CDR2, and V_{L}CDR3 sequences comprise SEQ ID NOs: 267, 268, and 269, respectively, including variants thereof;
the V_{H}CDR1, V_{H}CDR2, and V_{H}CDR3 sequences comprise SEQ ID NOs: 270, 271, and 272, respectively, and the V_{L}CDR1, V_{L}CDR2, and V_{L}CDR3 sequences comprise SEQ ID NOs: 273, 274, and 275, respectively, including variants thereof;
the V_{H}CDR1, V_{H}CDR2, and V_{H}CDR3 sequences comprise SEQ ID NOs: 276, 277, and 278, respectively, and the V_{L}CDR1, V_{L}CDR2, and V_{L}CDR3 sequences comprise SEQ ID NOs: 279, 280, and 281, respectively, including variants thereof;
the V_{H}CDR1, V_{H}CDR2, and V_{H}CDR3 sequences comprise SEQ ID NOs: 282, 283, and 284, respectively, and the V_{L}CDR1, V_{L}CDR2, and V_{L}CDR3 sequences comprise SEQ ID NOs: 285, 286, and 287, respectively, including variants thereof;
the V_{L}CDR1, V_{H}CDR2, and V_{H}CDR3 sequences comprise SEQ ID NOs: 288, 289, and 290, respectively, and the V_{L}CDR1, V_{L}CDR2, and V_{L}CDR3 sequences comprise SEQ ID NOs: 291, 292, and 293, respectively, including variants thereof;
the V_{H}CDR1, V_{H}CDR2, and V_{H}CDR3 sequences comprise SEQ ID NOs: 294, 295, and 296, respectively, and the V_{L}CDR1, V_{L}CDR2, and V_{L}CDR3 sequences comprise SEQ ID NOs: 297, 298, and 299, respectively, including variants thereof; and/or
the V_{H}CDR1, V_{H}CDR2, and V_{H}CDR3 sequences comprise SEQ ID NOs: 300, 301, and 302, respectively, and the V_{L}CDR1, V_{L}CDR2, and V_{L}CDR3 sequences comprise SEQ ID NOs: 303, 304, and 305, respectively, including variants thereof,
including affinity matured variants of the foregoing which specifically bind to the human HRS polypeptide, and wherein the therapeutic composition is substantially endotoxin-free.

2. The therapeutic composition of claim 1, wherein:
the V_{H} sequence comprises a sequence at least 80, 85, 90, 95, 97, 98, 99, or 100% identical to SEQ ID NO:30, and the V_{L} sequence comprises a sequence at least 80, 85, 90, 95, 97, 98, 99, or 100% identical to SEQ ID NO:31;
the V_{H} sequence comprises a sequence at least 80, 85, 90, 95, 97, 98, 99, or 100% identical to SEQ ID NO:32, and the V_{L} sequence comprises a sequence at least 80, 85, 90, 95, 97, 98, 99, or 100% identical to SEQ ID NO:33;
the V_{H} sequence comprises a sequence at least 80, 85, 90, 95, 97, 98, 99, or 100% identical to SEQ ID NO:34, and the V_{L} sequence comprises a sequence at least 80, 85, 90, 95, 97, 98, 99, or 100% identical to SEQ ID NO:35;
the V_{H} sequence comprises a sequence at least 80, 85, 90, 95, 97, 98, 99, or 100% identical to SEQ ID NO:306, and the V_{L} sequence comprises a sequence at least 80, 85, 90, 95, 97, 98, 99, or 100% identical to SEQ ID NO:307;
the V_{H} sequence comprises a sequence at least 80, 85, 90, 95, 97, 98, 99, or 100% identical to SEQ ID NO:308, and the V_{L} sequence comprises a sequence at least 80, 85, 90, 95, 97, 98, 99, or 100% identical to SEQ ID NO:309;
the V_{H} sequence comprises a sequence at least 80, 85, 90, 95, 97, 98, 99, or 100% identical to SEQ ID NO:310, and the V_{L} sequence comprises a sequence at least 80, 85, 90, 95, 97, 98, 99, or 100% identical to SEQ ID NO:311;
the V_{H} sequence comprises a sequence at least 80, 85, 90, 95, 97, 98, 99, or 100% identical to SEQ ID NO:312, and the V_{L} sequence comprises a sequence at least 80, 85, 90, 95, 97, 98, 99, or 100% identical to SEQ ID NO:313;
the V_{H} sequence comprises a sequence at least 80, 85, 90, 95, 97, 98, 99, or 100% identical to SEQ ID NO:314, and the V_{L}, sequence comprises a sequence at least 80, 85, 90, 95, 97, 98, 99, or 100% identical to SEQ ID NO:315;
the V_{H} sequence comprises a sequence at least 80, 85, 90, 95, 97, 98, 99, or 100% identical to SEQ ID NO:316, and the V_{L} sequence comprises a sequence at least 80, 85, 90, 95, 97, 98, 99, or 100% identical to SEQ ID NO:317;
the V_{H} sequence comprises a sequence at least 80, 85, 90, 95, 97, 98, 99, or 100% identical to SEQ ID NO:318, and the V_{L} sequence comprises a sequence at least 80, 85, 90, 95, 97, 98, 99, or 100% identical to SEQ ID NO:319;
the V_{H} sequence comprises a sequence at least 80, 85, 90, 95, 97, 98, 99, or 100% identical to SEQ ID NO:320, and the V_{L} sequence comprises a sequence at least 80, 85, 90, 95, 97, 98, 99, or 100% identical to SEQ ID NO:321;
the V_{H} sequence comprises a sequence at least 80, 85, 90, 95, 97, 98, 99 or 100% identical to SEQ ID NO:322, and the V_{L} sequence comprises a sequence at least 80, 85, 90, 95, 97, 98, 99, or 100% identical to SEQ ID NO:323;
the V_{H} sequence comprises a sequence at least 80, 85, 90, 95, 97, 98, 99, or 100%, identical to SEQ ID NO:324, and the V_{L} sequence comprises a sequence at least 80, 85, 90, 95, 97, 98, 99, or 100% identical to SEQ ID NO:325,
the V_{H} sequence comprises a sequence at least 80, 85, 90, 95, 97, 98, 99, or 100% identical to SEQ ID NO:326, and the V_{L} sequence comprises a sequence at least 80, 85, 90, 95, 97, 98, 99, or 100%, identical to SEQ ID NO:327;
the V_{H} sequence comprises a sequence at least 80, 85, 90, 95, 97, 98, 99, or 107% identical to SEQ ID NO:328, and the V_{L} sequence comprises a sequence at least 80, 85, 90, 95, 97, 98, 99, or 100% identical to SEQ ID NO:329;
the V_{H} sequence comprises a sequence at least 80, 85, 90, 95, 97, 98, 99, or 100% identical to SEQ ID NO:330, and the V_{L} sequence comprises a sequence at least 80, 85, 90, 95, 97, 98, 99, or 100% identical to SEQ ID NO:331;
the V_{H} sequence comprises a sequence at least 80, 85, 90, 95, 97, 98, 99, or 100% identical to SEQ ID NO:332, and the V_{L}, sequence comprises a sequence at least 80, 85, 90, 95, 97, 98, 99, or 100 identical to SEQ ID NO:333;
the V_{H} sequence comprises a sequence at least 80, 85, 90, 95, 97, 98, 99, or 100% identical to SEQ ID NO:334, and the V_{L} sequence comprises a sequence at least 80, 85, 90, 95, 97, 98, 99, or 100% identical to SEQ ID NO:335;
the V_{H} sequence comprises a sequence at least 80, 85, 90, 95, 97, 98, 99, or 100% identical to SEQ ID NO:336, and the V_{L} sequence comprises a sequence at least 80, 85, 90, 95, 97, 98, 99, or 100% identical to SEQ ID NO:337;
the V_{H} sequence comprises a sequence at least 80, 85, 90, 95, 97, 98, 99, or 100% identical to SEQ ID NO:338, and the V_{L} sequence comprises a sequence at least 80, 85, 90, 95, 97, 98, 99, or 100% identical to SEQ ID NO:339;
the V_{H} sequence comprises a sequence at least 80, 85, 90, 95, 97, 98, 99 or 100% identical to SEQ ID NO:340, and the V_{L} sequence comprises a sequence at least 80, 85, 90, 95, 97, 98, 99, or 100% identical to SEQ ID NO:341;
the V_{H} sequence comprises a sequence at least 80, 85, 90, 95, 97, 98, 99, or 100%, identical to SEQ ID NO:342, and the V_{L} sequence comprises a sequence at least 80, 85, 90, 95, 97, 98, 99, or 100% identical to SEQ ID NO:343,
the V_{H} sequence comprises a sequence at least 80, 85, 90, 95, 97, 98, 99, or 100% identical to SEQ ID NO:344, and the V_{L} sequence comprises a sequence at least 80, 85, 90, 95, 97, 98, 99, or 100%, identical to SEQ ID NO:345;
the V_{H} sequence comprises a sequence at least 80, 85, 90, 95, 97, 98, 99, or 107% identical to SEQ ID NO:346, and the V_{L} sequence comprises a sequence at least 80, 85, 90, 95, 97, 98, 99, or 100% identical to SEQ ID NO:347;
the V_{H} sequence comprises a sequence at least 80, 85, 90, 95, 97, 98, 99, or 100% identical to SEQ ID NO:348, and the V_{L} sequence comprises a sequence at least 80, 85, 90, 95, 97, 98, 99, or 100% identical to SEQ ID NO:349;
the V_{H} sequence comprises a sequence at least 80, 85, 90, 95, 97, 98, 99, or 100% identical to SEQ ID NO:350, and the V_{L}, sequence comprises a sequence at least 80, 85, 90, 95, 97, 98, 99, or 100 identical to SEQ ID NO:351;
the V_{H} sequence comprises a sequence at least 80, 85, 90, 95, 97, 98, 99, or 100% identical to SEQ ID NO:352, and the V_{L} sequence comprises a sequence at least 80, 85, 90, 95, 97, 98, 99, or 100% identical to SEQ ID NO:353;
the V_{H} sequence comprises a sequence at least 80, 85, 90, 95, 97, 98, 99, or 100% identical to SEQ ID NO:354, and the V_{L} sequence comprises a sequence at least 80, 85, 90, 95, 97, 98, 99, or 100% identical to SEQ ID NO:35S;
the V_{H} sequence comprises a sequence at least 80, 85, 90, 95, 97, 98, 99, or 100% identical to SEQ ID NO:356, and the V_{L} sequence comprises a sequence at least 80, 85, 90, 95, 97, 98, 99, or 100% identical to SEQ ID NO:357;
the V_{H} sequence comprises a sequence at least 80, 85, 90, 95, 97, 98, 99 or 100% identical to SEQ ID NO:358, and the V_{L} sequence comprises a sequence at least 80, 85, 90, 95, 97, 98, 99, or 100% identical to SEQ ID NO:359;
the V_{H} sequence comprises a sequence at least 80, 85, 90, 95, 97, 98, 99, or 100%, identical to SEQ ID NO:360, and the V_{L} sequence comprises a sequence at least 80, 85, 90, 95, 97, 98, 99, or 100% identical to SEQ ID NO:361,
the V_{H} sequence comprises a sequence at least 80, 85, 90, 95, 97, 98, 99, or 100% identical to SEQ ID NO:362, and the V_{L} sequence comprises a sequence at least 80, 85, 90, 95, 97, 98, 99, or 100%, identical to SEQ ID NO:363;
the V_{H} sequence comprises a sequence at least 80, 85, 90, 95, 97, 98, 99, or 107% identical to SEQ ID NO:364, and the V_{L} sequence comprises a sequence at least 80, 85, 90, 95, 97, 98, 99, or 100% identical to SEQ ID NO:365;
the V_{H} sequence comprises a sequence at least 80, 85, 90, 95, 97, 98, 99, or 100% identical to SEQ ID NO:366, and the V_{L} sequence comprises a sequence at least 80, 85, 90, 95, 97, 98, 99, or 100% identical to SEQ ID NO:367;
the V_{H} sequence comprises a sequence at least 80, 85, 90, 95, 97, 98, 99, or 100% identical to SEQ ID NO:368, and the V_{L}, sequence comprises a sequence at least 80, 85, 90, 95, 97, 98, 99, or 100 identical to SEQ ID NO:369;
the V_{H} sequence comprises a sequence at least 80, 85, 90, 95, 97, 98, 99, or 100% identical to SEQ ID NO:370, and the V_{L} sequence comprises a sequence at least 80, 85, 90, 95, 97, 98, 99, or 100% identical to SEQ ID NO:371;
the V_{H} sequence comprises a sequence at least 80, 85, 90, 95, 97, 98, 99, or 100% identical to SEQ ID NO:372, and the V_{L} sequence comprises a sequence at least 80, 85, 90, 95, 97, 98, 99, or 100% identical to SEQ ID NO:373;
the V_{H} sequence comprises a sequence at least 80, 85, 90, 95, 97, 98, 99, or 100% identical to SEQ ID NO:374, and the V_{L} sequence comprises a sequence at least 80, 85, 90, 95, 97, 98, 99, or 100% identical to SEQ ID NO:375;
the V_{H} sequence comprises a sequence at least 80, 85, 90, 95, 97, 98, 99 or 100% identical to SEQ ID NO:376, and the V_{L} sequence comprises a sequence at least 80, 85, 90, 95, 97, 98, 99, or 100% identical to SEQ ID NO:377;
the V_{H} sequence comprises a sequence at least 80, 85, 90, 95, 97, 98, 99, or 100%, identical to SEQ ID NO:378, and the V_{L} sequence comprises a sequence at least 80, 85, 90, 95, 97, 98, 99, or 100% identical to SEQ ID NO:379,
the V_{H} sequence comprises a sequence at least 80, 85, 90, 95, 97, 98, 99, or 100% identical to SEQ ID NO:380, and the V_{L} sequence comprises a sequence at least 80, 85, 90, 95, 97, 98, 99, or 100%, identical to SEQ ID NO:381;
the V_{H} sequence comprises a sequence at least 80, 85, 90, 95, 97, 98, 99, or 100% identical to SEQ ID NO:382, and the V_{L} sequence comprises a sequence at least 80, 85, 90, 95, 97, 98, 99, or 100% identical to SEQ ID NO:383;
the V_{H} sequence comprises a sequence at least 80, 85, 90, 95, 97, 98, 99, or 100% identical to SEQ ID NO:384, and the V_{L} sequence comprises a sequence at least 80, 85, 90, 95, 97, 98, 99, or 100% identical to SEQ ID NO:385;
the V_{H} sequence comprises a sequence at least 80, 85, 90, 95, 97, 98, 99, or 100% identical to SEQ ID NO:386, and the V_{L} sequence comprises a sequence at least 80, 85, 90, 95, 97, 98, 99, or 100%, identical to SEQ ID NO:387;
the V_{H} sequence comprises a sequence at least 80, 85, 90, 95, 97, 98, 99, or 100% identical to SEQ ID NO:388, and the V_{L} sequence comprises a sequence at least 80, 85, 90, 95, 97, 98, 99, or 100% identical to SEQ ID NO:389;
the V_{H} sequence comprises a sequence at least 80, 85, 90, 95, 97, 98, 99, or 100% identical to SEQ ID NO:390, and the V_{L} sequence comprises a sequence at least 80, 85, 90, 95, 97, 98, 99, or 100% identical to SEQ ID NO:391;
the V_{H} sequence comprises a sequence at least 80, 85, 90, 95, 97, 98, 99, or 100% identical to SEQ ID NO:392, and the V_{L} sequence comprises a sequence at least 80, 85, 90, 95, 97, 98, 99, or 100% identical to SEQ ID NO:393; and/or
the V_{H} sequence comprises a sequence at least 80, 85, 90, 95, 97, 98, 99, or 100% identical to SEQ ID NO:394, and the V_{L} sequence comprises a sequence at least 80, 85, 90, 95, 97, 98, 99, or 100% identical to SEQ ID NO:395

3-10. (canceled)

11. The therapeutic composition of claim 1, wherein the at least one antibody or antigen-binding fragment thereof interferes with binding of the human HRS polypeptide to a human neuropilin-2 (NP2) polypeptide selected from Table N1.

12-14. (canceled)

15. The therapeutic composition of claim 11, wherein the at least one antibody or antigen-binding fragment thereof is a blocking antibody which inhibits about or at least about 80-100% of the theoretical maximal binding between the HRS polypeptide and the NP2 polypeptide after pro-incubation with the HRS polypeptide in a stoichiometrically equivalent amount, optionally about or at least about 80, 85, 90, 95, or 100% of the theoretical maximal binding.

16. The therapeutic composition of claim 11, wherein the at least one antibody or antigen-binding fragment thereof is a partial-blocking antibody which inhibits about 20-80% of the theoretical maximal binding between the HRS polypeptide and the NP2 polypeptide after pre-incubation with the HRS polypeptide in a stoichiometrically equivalent amount, optionally about or at least about 20, 25, 30, 35, 40, 45, 50, 55, 60, 65, 70, 75, or 80% of the theoretical maximal binding.

17. The therapeutic composition of claim 1, wherein the at least one antibody or antigen-binding fragment thereof is a non-blocking antibody which inhibits about or less than about 10% of the theoretical maximal binding between the HRS polypeptide and the NP2 polypeptide after pre-incubation with the HRS polypeptide in a stoichiometrically equivalent amount.

18-21. (canceled)

22. The therapeutic composition of claim 1, wherein the at least one antibody or antigen-binding fragment thereof comprises an IgA (including subclasses IgA1 and IgA2) IgD, IgE, IgG (including subclasses IgG1, IgG2, IgG3, and IgG4), or IgM Fc domain, optionally a human Fc domain, or a hybrid and/or variant thereof.

23. The therapeutic composition of claim 22, wherein the at least one antibody or antigen-binding fragment thereof comprises an IgG Fc domain with high effector function in humans, optionally an IgG1 or IgG3 Fc domain.

24. The therapeutic composition of claim 22, wherein the at least one antibody or antigen-binding fragment thereof comprises an IgG Fc domain with low effector function in humans, optionally an IgG2 or IgG4 Fc domain.

25. The therapeutic composition of claim 24, wherein the at least one antibody or antigen-binding fragment thereof comprises an IgG1 or IgG4 Fc domain, optionally selected from Table F1.

26-30. (canceled)

31. The therapeutic composition of claim 1, wherein the at least one antibody or antigen-binding fragment thereof is a monoclonal antibody.

32. The therapeutic composition of claim 1, wherein the at least one antibody or antigen-binding fragment thereof is a humanized antibody.

33. The therapeutic composition of claim 1, wherein the at least one antibody or antigen-binding fragment thereof is an Fv fragment, a single chain Fv (scFv) polypeptide, an adnectin, an anticalin, an aptamer, an avimer, a camelid antibody, a designed ankyrin repeat protein (DARPin) a minibody, a nanobody, or a unibody.

34-48. (canceled)

49. The therapeutic composition of claim 1, wherein the composition has a purity of at least about 80%, 85%, 90%, 95%, 98%, or 99% on a protein basis with respect to the at least one antibody or antigen-binding fragment, and is substantially aggregate-free

50. (canceled)

51. The therapeutic composition of claim 1, wherein the therapeutic composition is a sterile, injectable solution, optionally suitable for intravenous, intramuscular, subcutaneous, or intraperitoneal administration.

52. The therapeutic composition of claim 1, further comprising at least one cancer immunotherapy agent selected from one or more of:
a Programmed Death-Ligand 1 (PD-L1) and/or Programmed Death-Ligand 2 (PD-L2) antagonist optionally selected from one or more of an antibody or antigen-binding fragment or small molecule that specifically binds thereto, atezolizumab (MPDL3280A), avelumab (MSB0010718C), and durvalumab (MEDI4736);
a Programmed Death 1 (PD-1) antagonist optionally selected from one or more of an antibody or antigen-binding fragment or small molecule that specifically binds thereto, nivolumab, nembrolizumab, MK-3475, AMP-224, AMP-514, PDR001, and pidilizumab;
a Cytotoxic T-Lymphocyte-Associated protein 4 (CTLA-4) antagonist optionally selected from one or more of an antibody or antigen-binding fragment or small molecule that specifically binds thereto, ipilimumab, and tremelimumab; and
an Indoleamine 2,3-dioxygenase (IDO), tryptophan 2,3-dioxygenase (IDO) antagonist optionally selected from one or more of an antibody or antigen-binding fragment or small molecule that specifically binds thereto, indoximod (NLG-8189, 1-methyl-tryptophan (1MT), β-Carboline (norharmane: 9H-pyrido[3,4-b]indole), rosmarinic acid, and epacadostat.

53-80. (canceled)

81. A method of treating a cancer in a subject in need thereof, comprising administering to the subject a therapeutic composition according to claim 1.

82. The method of claim 81, for reducing or preventing re-emergence of a cancer in a subject in need thereof, wherein administration of the therapeutic composition enables generation of an immune memory to the cancer.

83. The method of claim 81, wherein the subject has or is at risk for developing diabetes.

84. The method of claim 81, comprising administering to the subject at least one cancer immunotherapy agent selected from one or more of:
a Programmed Death-Ligand 1 (PD-L1) and/or Programmed Death-Ligand 2 (PD-L2) antagonist optionally selected from one or more of an antibody or antigen-binding fragment or small molecule that specifically binds thereto, atezolizumab (MPDL3280A), avelumab (MSB0010718C), and durvalumab (MEDI47361);
a Programmed Death 1 (PD-1) antagonist optionally selected from one or more of an antibody or antigen-binding fragment or small molecule that specifically binds thereto, nivolumab, pembrolizumab, MK-3475, AMP-224, AMP-514, PDR001, and pidilizumab;
a Cytotoxic T-Lymphocyte-Associated protein 4 (CTLA-4) antagonist optionally selected from one or more of an antibody or antigen-binding fragment or small molecule that specifically binds thereto, ipilimumab, and tremelimumab; and
an Indoleamine 2,3-dioxygenase (IDO), tryptophan 2,3-dioxygenase (IDO) antagonist optionally selected from one or more of an antibody or antigen-binding fragment or small molecule that specifically binds thereto, indoximod (NLG-8189), 1-methyl-tryptophan (1MT), β-Carboline (norharmane: 9H-pyrido[3,4-b]indole), rosmarinic acid, and euacadostat.

85. The method of claim 84, wherein the at least one anti-HRS antibody and the at least one cancer immunotherapy agent are administered separately, as separate compositions.

86. The method of claim 84, wherein the at least one anti-HRS antibody and the at least one cancer immunotherapy agent are administered together as part of the same therapeutic composition.

87-118. (canceled)

119. The method of claim 81, wherein the cancer is a primary cancer.

120. The method of claim 81, wherein the cancer is a metastatic cancer.

121. The method of claim 81, wherein the cancer is selected from one or more of melanoma (e.g., metastatic melanoma), pancreatic cancer, bone cancer, prostate cancer, small cell lung cancer, non-small cell lung cancer (NSCLC), mesothdioma, leukemia (e.g., lymphocytic leukemia, chronic myelogenous leukemia, acute myeloid leukemia, relapsed acute myeloid leukemia), lymphoma, hepatoma (hepatocellular carcinoma), sarcoma, B-cell malignancy, breast cancer, ovarian cancer, colorectal cancer, glioam, glioblastoma multiforme, meningioma, pituitary adenoma, vestibular schwannoma, primary CNS lymphoma, primitive neuroectodermal tumor (medulloblastoma) kidney cancer (e.g., renal cell carcinoma), bladder cancer, uterine cancer, esophageal cancer, brain cancer, head and neck cancers, cervical cancer, testicular cancer, thyroid cancer, and stomach cancer.

122. The method of claim 120, wherein the metastatic cancer is selected from one or more of:
(a) a bladder cancer which has metastasized to the bone, liver, and/or lungs;
(b) a breast cancer which has metastasized to the bone, brain, liver, and/or lungs;
(c) a colorectal cancer which has metastasized to the liver, lungs, and/or peritoneum;
(d) a kidney cancer which has metastasized to the adrenal glands, bone, brain, liver, and/or lungs;
(e) a lung cancer which has metastasized to the adrenal glands, bone, brain, liver, and/or other lung sites;
(f) a melanoma which has metastasized to the bone, brain, liver, lung, and/or skin/muscle;
(g) a ovarian cancer which has metastasized to the liver, lung, and/or peritoneum;
(h) a pancreatic cancer which has metastasized to the liver, lung, and/or peritoneum;
(i) a prostate cancer which has metastasized to the adrenal glands, bone, liver, and/or lungs;
(j) a stomach cancer which has metastasized to the liver, lung, and/or peritoneum;
(l) a thyroid cancer which has metastasized to the bone, liver, and/or lungs; and
(m) a uterine cancer which has metastasized to the bone, liver, lung, peritoneum, and/or vagina.

123. The method of claim 81, wherein the subject has, and/or is selected for treatment based on having, increased circulating or serum levels of at least one HRS polypeptide (optionally selected from Table H1), either bound or free, relative to the levels of a healthy or matched control standard or population of subject(s), optionally about or at least about 30, 40, 50, 60, 70, 80, 90, 100, 200, 300, 400, 500, 600, 700, 800, 900, 1000, 1100, 1200, 1300, 1400, 1500, 1600, 1700, 1800, 1900, 2000, 3000, 4000, or 5000 pM of the at least one HRS polypeptide, or about or at least about 30-100, 40-100, 50-100, 30-2000, 40-2000, 50-2000, 60-2000, 70-2000, 80-2000, 90-2000, 100-2000, 200-2000, 300-2000, 400-2000, 500-2000, 600-2000, 700-2000, 800-2000, 900-2000, 1000-2000, 2000-3000, 3000-4000, or 4000-5000 pM of the at least one HRS polypeptide.

124. The method of claim 81, wherein the subject has, and/or is selected for treatment based on having, a cancer which has increased levels or expression of an HRS polypeptide (optionally selected from Table H1) and/or a coding mRNA thereof relative to a non-cancerous control cell or tissue, optionally relative to a non-cancerous cell or tissue of the same type as the cancer, optionally wherein the HRS polypeptide is a splice variant selected from SV9, SV11, and SV14.

125. The method of claim 81, wherein the subject has, and/or is selected for treatment based on having, increased circulating or serum levels of a soluble neuropilin 2 (NP2) polypeptide (optionally selected from Table NI), either bound or free, relative to the levels of a healthy or matched control standard or population of subject(s), optionally circulating or serum levels of about or at least about 10, 20, 30, 50, 100, 200, 300, 400, 500, 600, 700, 800, 900, 1000, 1100, 1200, 1300, 1400, 1500, 1600, 1700, 1800, 1900, 2000, 3000, 4000, 5000 pM of the soluble NP2 polypeptide, or optionally circulating or serum levels about 30-50, 50-100, 100-2000, 200-2000, 300-2000, 400-2000, 500-2000, 600-2000, 700-2000, 800-2000, 900-2000, 1000-2000, 2000-3000, 3000-4000, 4000-5000 pM of the soluble NP2 polypeptide.

126. The method of claim 81, wherein the subject has, and/or is selected for treatment based on having, a cancer which has increased levels or expression of an NP2 polypeptide (optionally selected from Table NI) and/or a coding mRNA thereof relative to a non-cancerous control cell or tissue, optionally relative to a non-cancerous cell or tissue of the same type as the cancer.

127. The method of claim 81, wherein the subject has, and/or is selected for treatment based on having, increased circulating levels of HRS:NP2 complexes relative to a healthy or matched control standard or population of subject(s).

128. The method of claim 81, comprising administering the at least one anti-HRS antibody in an amount and at a frequency sufficient to reduce the average or maximum levels of at least one serum or free, circulating HRS polypeptide (optionally selected from Table H1) to about or less than about 500 pM, 400 pM, 300 pM, 200 pM, 100 pM, 50 pm, 40 pM, 30 pM, 20 pM, or 10 pM.

129. The method of claim 81, comprising administering the at least one anti-HRS antibody in an amount and at a frequency sufficient to achieve an average, sustained serum or circulating levels of a soluble NP2 polypeptide of about or less than about 500 pM, 400 pM, 300 pM, 200 pM, 100 pM, 50 pm, 40 pM, 30 pM, 20 pM, or 10 pM.

130. The method of claim 81, comprising administering the at least one anti-HRS antibody in an amount and at a frequency sufficient to achieve a reduction in the circulating levels of HRS:NP2 complexes, optionally a reduction of about or at least about 5, 10, 15, 20, 25, 30, 35, 40, 45, 50, 60, 70, 80, 90, 95, 99, or 100%.

131. The method claim 81, wherein the at least one anti-HRS antibody enhances the immune response to the cancer by about, or at least about, 5, 10, 15, 20, 25, 30, 35, 40, 45, 50, 60, 70, 80, 90, 100, 200, 300, 400, 500, 600, 700, 800, 900, 1000, 2000% or more, relative to a control.

132. The method of claim 84, wherein the at least one anti-HRS antibody enhances an anti-tumor and/or immunostimulatory activity of the cancer immunotherapy agent by about, or at least about, 5, 10, 15, 20, 25, 33, 40, 45, 50, 60, 70, 80, 90, 100, 200, 300, 400, 500, 600, 700, 800, 900, 1000, 2000% or more, relative to the cancer immunotherapy agent alone.

133. The method of claim 81, comprising administering the at least one anti-HRS antibody in an amount and at a frequency sufficient to achieve a steady state concentration, or average circulating concentration, of the at least one anti-HRS antibody of between about 1 nM and about 1 μM, between about 1 nM and about 100 nM, between about 1 nM and about 10 nM, or between about 1 nM and about 3 μM

134-135. (canceled)

136. A patient care kit, comprising:
(a) at least one antibody or antigen-binding fragment thereof that specifically binds to a human histidyl-tRNA synthetase (HRS) polypeptide (an anti-HRS antibody) and comprises a heavy chain variable region (V_{H}) sequence that comprises complementary determining region V_{H}CDR1, V_{H}CDR2, and V_{H}CDR3 sequences, and a light chain variable region (V_{L}) sequence that comprises complementary determining region V_{L}CDR1, V_{L}CDR2, and V_{L}CDR3 sequences wherein:
the V_{H}CDR1, V_{H}CDR2, and V_{H}CDR3 sequences comprise the consensus sequences of SEQ ID NOs:396, 397, and 398 (as defined in Table A3), respectively, and the V_{L}CDR1, V_{L}CDR2, and V_{L}CDR3 sequences comprise the consensus sequences SEQ ID NOs: 399, 400, and 401 (as defined in Table A3), respectively, including variants thereof;
the V_{H}CDR1, V_{H}CDR2, and V_{H}CDR3 sequences comprise the consensus sequences of SEQ ID NOs: 402, 403, and 404 (as defined in Table A3, respectively, and the V_{L}CDR1, V_{L}CDR2, and V_{L}CDR3 sequences comprise the consensus sequences of SEQ ID NOs: 405, 406, and 407 (as defined in Table A3), respectively, including variants thereof;
the V_{H}CDR1, V_{H}CDR2, and V_{H}CDR3 sequences comprise the consensus sequences of SEQ ID NOs: 408, 409, and 410 (as defined in Table A3), respectively, and the V_{L}CDR1, V_{L}CDR2, and V_{L}CDR3 sequences comprise the consensus sequences of SEQ ID NOs: 411, 412, and 413 (as defined in Table A3), respectively, including variants thereof;
the V_{H}CDR1, V_{H}CDR2, and V_{H}CDR3 sequences comprise SEQ ID NOs: 12, 13, and 14, respectively, and the V_{L}CDR1, V_{L}CDR2, and V_{L}CDR3 sequences comprise SEQ ID NOs: 15, 16, and 17, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 18, 19, and 20, respectively, and the V_{L}CDR1, V_{L}CDR2, and V_{L}CDR3 sequences comprise SEQ ID NOs: 21, 22, and 23, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 24, 25, and 26, respectively, and the V_{L}CDR1, V_{L}CDR2, and V_{L}CDR3 sequences comprise SEQ ID NOs, 27, 28, and 29, respectively, including variants thereof with 1, 2, 3, 4, or 5 alterations in the CDR(s) and which specifically bind to the human HRS polypeptide;
the V_{H}CDR1, V_{H}CDR2, and V_{H}CDR3 sequences comprise SEQ ID NOs: 36, 37, and 38, respectively, and the V_{L}CDR1, V_{L}CDR2, and V_{L}CDR3 sequences comprise SEQ ID NOs: 39, 40, and 41, respectively, including variants thereof;
the V_{H}CDR1, V_{H}CDR2, and V_{H}CDR3 sequences comprise SEQ ID NOs: 42, 43, and 44, respectively, and the V_{L}CDR1, V_{L}CDR2, and V_{L}CDR3 sequences comprise SEQ ID NOs: 45 46, and 47, respectively, including variants thereof;
the V_{H}CDR1, V_{H}CDR2, and V_{H}CDR3 sequences comprise SEQ ID NOs: 48, 49, and 50, respectively, and the V_{L}CDR1, V_{L}CDR2, and V_{L}CDR3 sequences comprise SEQ ID NOs, 51, 52, and 53, respectively, including variants thereof;
the V_{H}CDR1, V_{H}CDR2, and V_{H}CDR3 sequences comprise SEQ ID NOs: 54, 55, and 56, respectively, and the V_{L}CDR1, V_{L}CDR2, and V_{L}CDR3 sequences comprise SEQ ID NOs: 57, 58, and 59, respectively, including variants thereof;
the V_{H}CDR1, V_{H}CDR2, and V_{H}CDR3 sequences comprise SEQ ID NOs: 60, 61, and 62, respectively, and the V_{L}CDR1, V_{L}CDR2, and V_{L}CDR3 sequences comprise SEQ ID NOS: 63, 64, and 65, respectively, including variants thereof;
the V_{H}CDR1, V_{H}CDR2, and V_{H}CDR3 sequences comprise SEQ ID NOs: 66, 67, and 68, respectively, and the V_{L}CDR1, V_{L}CDR2, and V_{L}CDR3 sequences comprise SEQ ID NOs: 69, 70, and 71, respectively, including variants thereof;
the V_{H}CDR1, V_{H}CDR2, and V_{H}CDR3 sequences comprise SEQ ID NOs: 72, 73, and 74, respectively, and the V_{L}CDR1, V_{L}CDR2, and V_{L}CDR3 sequences comprise SEQ ID NOs: 75, 76, and 77, respectively, including variants thereof;
the V_{H}CDR1, V_{H}CDR2, and V_{H}CDR3 sequences comprise SEQ ID NOs: 78, 79, and 80, respectively, and the V_{L}CDR1, V_{L}CDR2, and V_{L}CDR3 sequences comprise SEQ ID NOs: 81, 82, and 83, respectively, including variants thereof;
the V_{H}CDR1, V_{H}CDR2, and V_{H}CDR3 sequences comprise SEQ ID NOs: 84, 85, and 86, respectively, and the V_{L}CDR1, V_{L}CDR2, and V_{L}CDR3 sequences comprise SEQ ID NOs, 87, 88, and 89, respectively, including variants thereof;
the V_{H}CDR1, V_{H}CDR2, and V_{H}CDR3 sequences comprise SEQ ID NOs: 90, 91, and 92, respectively, and the V_{L}CDR1, V_{L}CDR2, and V_{L}CDR3 sequences comprise SEQ ID NOs: 93, 94, and 95, respectively, including variants thereof;
the V_{H}CDR1, V_{H}CDR2, and V_{H}CDR3 sequences comprise SEQ ID NOs: 96, 97, and 98, respectively, and the V_{L}CDR1, V_{L}CDR2, and V_{L}CDR3 sequences comprise SEQ ID NOs, 99, 100, and 101, respectively, including variants thereof;
the V_{H}CDR1, V_{H}CDR2, and V_{H}CDR3 sequences comprise SEQ ID NOs: 102, 103, and 104, respectively, and the V_{L}CDR1, V_{L}CDR2, and V_{L}CDR3 sequences comprise SEQ ID NOs, 105, 106, and 107, respectively, including variants thereof;
the V_{H}CDR1, V_{H}CDR2, and V_{H}CDR3 sequences comprise SEQ ID NOs: 108, 109, and 110, respectively, and the V_{L}CDR1, V_{L}CDR2, and V_{L}CDR3 sequences comprise SEQ ID NOs: 111, 112, and 113, respectively, including variants thereof,
the V_{H}CDR1, V_{H}CDR2, and V_{H}CDR3 sequences comprise SEQ ID NOs: 114, 115, and 116, respectively, and the V_{L}CDR1, V_{L}CDR2, and V_{L}CDR3 sequences comprise SEQ ID NOs, 117, 118, and 119, respectively, including variants thereof;
the V_{H}CDR1, V_{H}CDR2, and V_{H}CDR3 sequences comprise SEQ ID NOs: 120, 121, and 122, respectively, and the V_{L}CDR1, V_{L}CDR2, and V_{L}CDR3 sequences comprise SEQ ID NOs: 123, 124, and 125, respectively, including variants thereof;
the V_{H}CDR1, V_{H}CDR2, and V_{H}CDR3 sequences comprise SEQ ID NOs: 126, 127, and 128, respectively, and the V_{L}CDR1, V_{L}CDR2, and V_{L}CDR3 sequences comprise SEQ ID NOs: 129, 130, and 131, respectively, including variants thereof;
the V_{H}CDR1, V_{H}CDR2, and V_{H}CDR3 sequences comprise SEQ ID NOs: 132, 133, and 134, respectively, and the V_{L}CDR1, V_{L}CDR2, and V_{L}CDR3 sequences comprise SEQ ID NOs: 135, 136, and 137, respectively, including variants thereof;
the V_{H}CDR1, V_{H}CDR2, and V_{H}CDR3 sequences comprise SEQ ID NOs: 138, 139, and 140, respectively, and the V_{L}CDR1, V_{L}CDR2, and V_{L}CDR3 sequences comprise SEQ ID NOS: 141, 142, and 143, respectively, including variants thereof;
the V_{H}CDR1, V_{H}CDR2, and V_{H}CDR3 sequences comprise SEQ ID NOs: 144, 145, and 146, respectively, and the V_{L}CDR1, V_{L}CDR2, and V_{L}CDR3 sequences comprise SEQ ID NOs, 147, 148, and 149, respectively, including variants thereof;
the V_{H}CDR1, V_{H}CDR2, and V_{H}CDR3 sequences comprise SEQ ID NOs: 150, 151, and 152, respectively, and the V_{L}CDR1, V_{L}CDR2, and V_{L}CDR3 sequences comprise SEQ ID NOs: 153, 154, and 155, respectively, including variants thereof;
the V_{L}CDR1, V_{H}CDR2, and V_{H}CDR3 sequences comprise SEQ ID NOs: 156, 157, and 158, respectively, and the V_{L}CDR1, V_{L}CDR2, and V_{L}CDR3 sequences comprise SEQ ID NOs, 159, 160, and 161, respectively, including variants thereof;
the V_{H}CDR1, V_{H}CDR2, and V_{H}CDR3 sequences comprise SEQ ID NOs, 162, 163, and 164, respectively, and the V_{L}CDR1, V_{L}CDR2, and V_{L}CDR3 sequences comprise SEQ ID NOs: 165, 166, and 167, respectively, including variants thereof,
the V_{H}CDR1, V_{H}CDR2, and V_{H}CDR3 sequences comprise SEQ ID NOs: 168, 169, and 170, respectively, and the V_{L}CDR1, V_{L}CDR2, and V_{L}CDR3 sequences comprise SEQ ID NOs, 171, 172, and 173, respectively, including variants thereof;
the V_{H}CDR1, V_{H}CDR2, and V_{H}CDR3 sequences comprise SEQ ID NOs: 174, 175, and 176, respectively, and the V_{L}CDR1, V_{L}CDR2, and V_{L}CDR3 sequences comprise SEQ ID NOs: 177, 178, and 179, respectively, including variants thereof;
the V_{H}CDR1, V_{H}CDR2, and V_{H}CDR3 sequences comprise SEQ ID NOs: 180, 181, and 182, respectively, and the V_{L}CDR1, V_{L}CDR2, and V_{L}CDR3 sequences comprise SEQ ID NOs: 183, 184, and 185, respectively, including variants thereof;
the V_{H}CDR1, V_{H}CDR2, and V_{H}CDR3 sequences comprise SEQ ID NOs: 186, 187, and 188, respectively, and the V_{L}CDR1, V_{L}CDR2, and V_{L}CDR3 sequences comprise SEQ ID NOs: 189, 190, and 191, respectively, including variants thereof;
the V_{H}CDR1, V_{H}CDR2, and V_{H}CDR3 sequences comprise SEQ ID NOs: 192, 193, and 194, respectively, and the V_{L}CDR1, V_{L}CDR2, and V_{L}CDR3 sequences comprise SEQ ID NOS: 195, 196, and 197, respectively, including variants thereof;
the V_{H}CDR1, V_{H}CDR2, and V_{H}CDR3 sequences comprise SEQ ID NOs: 198, 199, and 200, respectively, and the V_{L}CDR1, V_{L}CDR2, and V_{L}CDR3 sequences comprise SEQ ID NOs, 201, 202, and 203, respectively, including variants thereof;
the V_{H}CDR1, V_{H}CDR2, and V_{H}CDR3 sequences comprise SEQ ID NOs: 204, 205, and 206, respectively, and the V_{L}CDR1, V_{L}CDR2, and V_{L}CDR3 sequences comprise SEQ ID NOs: 207, 208, and 209, respectively, including variants thereof;
the V_{H}CDR1, V_{H}CDR2, and V_{H}CDR3 sequences comprise SEQ ID NOs: 210, 211, and 212, respectively, and the V_{L}CDR1, V_{L}CDR2, and V_{L}CDR3 sequences comprise SEQ ID NOs: 213, 214, and 213, respectively, including variants thereof;
the V_{L}CDR1, V_{H}CDR2, and V_{H}CDR3 sequences comprise SEQ ID NOs: 216, 217, and 218, respectively, and the V_{L}CDR1, V_{L}CDR2, and V_{L}CDR3 sequences comprise SEQ ID NOs: 219, 220, and 221, respectively, including variants thereof,
the V_{H}CDR1, V_{H}CDR2, and V_{H}CDR3 sequences comprise SEQ ID NOs: 222, 223, and 224, respectively, and the V_{L}CDR1, V_{L}CDR2, and V_{L}CDR3 sequences comprise SEQ ID NOs, 225, 226, and 227, respectively, including variants thereof;
the V_{H}CDR1, V_{H}CDR2, and V_{H}CDR3 sequences comprise SEQ ID NOs: 228, 229, and 230, respectively, and the V_{L}CDR1, V_{L}CDR2, and V_{L}CDR3 sequences comprise SEQ ID NOs: 231, 232, and 233, respectively, including variants thereof;
the V_{H}CDR1, V_{H}CDR2, and V_{H}CDR3 sequences comprise SEQ ID NOs: 234, 235, and 236, respectively, and the V_{L}CDR1, V_{L}CDR2, and V_{L}CDR3 sequences comprise SEQ ID NOs: 237, 238, and 239, respectively, including variants thereof;
the V_{H}CDR1, V_{H}CDR2, and V_{H}CDR3 sequences comprise SEQ ID NOs: 240, 241, and 242, respectively, and the V_{L}CDR1, V_{L}CDR2, and V_{L}CDR3 sequences comprise SEQ ID NOs: 243, 244, and 245, respectively, including variants thereof;
the V_{H}CDR1, V_{H}CDR2, and V_{H}CDR3 sequences comprise SEQ ID NOs: 246, 247, and 248, respectively, and the V_{L}CDR1, V_{L}CDR2, and V_{L}CDR3 sequences comprise SEQ ID NOS: 249, 250, and 251, respectively, including variants thereof;
the V_{H}CDR1, V_{H}CDR2, and V_{H}CDR3 sequences comprise SEQ ID NOs: 252, 253, and 254, respectively, and the V_{L}CDR1, V_{L}CDR2, and V_{L}CDR3 sequences comprise SEQ ID NOs, 255, 256, and 257, respectively, including variants thereof;
the V_{H}CDR1, V_{H}CDR2, and V_{H}CDR3 sequences comprise SEQ ID NOs: 258, 259, and 260, respectively, and the V_{L}CDR1, V_{L}CDR2, and V_{L}CDR3 sequences comprise SEQ ID NOs: 261, 262, and 263, respectively, including variants thereof;
the V_{H}CDR1, V_{H}CDR2, and V_{H}CDR3 sequences comprise SEQ ID NOs: 264, 265, and 266, respectively, and the V_{L}CDR1, V_{L}CDR2, and V_{L}CDR3 sequences comprise SEQ ID NOs, 267, 268, and 269, respectively, including variants thereof;
the V_{H}CDR1, V_{H}CDR2, and V_{H}CDR3 sequences comprise SEQ ID NOs, 270, 271, and 272, respectively, and the V_{L}CDR1, V_{L}CDR2, and V_{L}CDR3 sequences comprise SEQ ID NOs: 273, 274, and 275, respectively, including variants thereof,
the V_{H}CDR1, V_{H}CDR2, and V_{H}CDR3 sequences comprise SEQ ID NOs: 276, 277, and 278, respectively, and the V_{L}CDR1, V_{L}CDR2, and V_{L}CDR3 sequences comprise SEQ ID NOs, 279, 280, and 281, respectively, including variants thereof,
the V_{H}CDR1, V_{H}CDR2, and V_{H}CDR3 sequences comprise SEQ ID NOs: 282, 283, and 284, respectively, and the V_{L}CDR1, V_{L}CDR2, and V_{L}CDR3 sequences comprise SEQ ID NOs: 285, 286, and 287, respectively, including variants thereof;
the V_{H}CDR1, V_{H}CDR2, and V_{H}CDR3 sequences comprise SEQ ID NOs: 288, 289, and 290, respectively, and the V_{L}CDR1, V_{L}CDR2, and V_{L}CDR3 sequences comprise SEQ ID NOs: 291, 292, and 293, respectively, including variants thereof;
the V_{H}CDR1, V_{H}CDR2, and V_{H}CDR3 sequences comprise SEQ ID NOs: 294, 295, and 296, respectively, and the V_{L}CDR1, V_{L}CDR2, and V_{L}CDR3 sequences comprise SEQ ID NOs: 297, 298, and 299, respectively, including variants thereof; and/or
the V_{H}CDR1, V_{H}CDR2, and V_{H}CDR3 sequences comprise SEQ ID NOs: 300, 301, and 302, respectively, and the V_{L}CDR1, V_{L}CDR2, and V_{L}CDR3 sequences comprise SEQ ID NOs: 303, 304, and 305, respectively, including variants thereof,
including affinity matured variants of the foregoing which specifically bind to the human HRS polypeptide; and optionally
(b) at least one cancer immunotherapy agent.

137. The patient care kit of claim 136, wherein (a) and (b) are in separate therapeutic compositions.

138. The patient care kit of claim 136, wherein (a) and (b) are in the same therapeutic composition.

139-160. (canceled)


SEQUENCE LISTING (816 sequences)

SEQ ID NO: 1 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 2 - protein, 500 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKR

SEQ ID NO: 3 - protein, 501 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 4 - protein, 502 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRT

SEQ ID NO: 5 - protein, 503 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTG

SEQ ID NO: 6 - protein, 504 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQ

SEQ ID NO: 7 - protein, 505 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQP

SEQ ID NO: 8 - protein, 506 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 9 - protein, 505 aa, Homo sapiens
AERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPL

SEQ ID NO: 10 - protein, 507 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLC

SEQ ID NO: 11 - protein, 508 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCI

SEQ ID NO: 12 - protein, 10 aa, Artificial Sequence
  Made in Lab - Synthesized CDR sequence
GYTFTDYCIG

SEQ ID NO: 13 - protein, 17 aa, Artificial Sequence
  Made in Lab - Synthesized CDR sequence
DICPGDTYTNDNEKFKD

SEQ ID NO: 14 - protein, 13 aa, Artificial Sequence
  Made in Lab - Synthesized CDR sequence
GEEQLGLRNAMDY

SEQ ID NO: 15 - protein, 14 aa, Artificial Sequence
  Made in Lab - Synthesized CDR sequence
QSQSVSTSTYNYMH

SEQ ID NO: 16 - protein, 7 aa, Artificial Sequence
  Made in Lab - Synthesized CDR sequence
YASNLES

SEQ ID NO: 17 - protein, 9 aa, Artificial Sequence
  Made in Lab - Synthesized CDR sequence
GHSYEIPWT

SEQ ID NO: 18 - protein, 10 aa, Artificial Sequence
  Made in Lab - Synthesized CDR sequence
GFTFSDYYMT

SEQ ID NO: 19 - protein, 17 aa, Artificial Sequence
  Made in Lab - Synthesized CDR sequence
YISGSFRYTNYADKVKG

SEQ ID NO: 20 - protein, 11 aa, Artificial Sequence
  Made in Lab - Synthesized CDR sequence
YVYQVVAIGDL

SEQ ID NO: 21 - protein, 11 aa, Artificial Sequence
  Made in Lab - Synthesized CDR sequence
RASQGISSWLA

SEQ ID NO: 22 - protein, 7 aa, Artificial Sequence
  Made in Lab - Synthesized CDR sequence
AASSLQS

SEQ ID NO: 23 - protein, 9 aa, Artificial Sequence
  Made in Lab - Synthesized CDR sequence
QQAESFPYT

SEQ ID NO: 24 - protein, 10 aa, Artificial Sequence
  Made in Lab - Synthesized CDR sequence
GFTFSDYYMS

SEQ ID NO: 25 - protein, 17 aa, Artificial Sequence
  Made in Lab - Synthesized CDR sequence
YISDKSRYTKYTDKVRG

SEQ ID NO: 26 - protein, 11 aa, Artificial Sequence
  Made in Lab - Synthesized CDR sequence
YLYQVIAIADA

SEQ ID NO: 27 - protein, 11 aa, Artificial Sequence
  Made in Lab - Synthesized CDR sequence
RASQGISSWLA

SEQ ID NO: 28 - protein, 7 aa, Artificial Sequence
  Made in Lab - Synthesized CDR sequence
VASNLES

SEQ ID NO: 29 - protein, 9 aa, Artificial Sequence
  Made in Lab - Synthesized CDR sequence
QQAESFPYT

SEQ ID NO: 30 - protein, 122 aa, Artificial Sequence
  Made in Lab - Synthesized heavy chain variable region sequence
QVQLVQSGAEVKKPGASVKVSCKASGYTFTDYCIGWIRQAPGQGLEWMGDICPGDTYTNDNEKFKDRATMTADTSTSTAYMELSSLRSEDTAVYYCARGEEQLGLRNAMDYWGQGTLVTVSS

SEQ ID NO: 31 - protein, 110 aa, Artificial Sequence
  Made in Lab - Synthesized light chain variable region sequence
EIVLTQSPATLSLSPGERATLSCQSQSVSTSTYNYMHWYQQKPGQAPRLLIKYASNLESGIPDRFSGSGSGTDFTLTISRLEPEDFATYYCGHSYEIPWTFGGGTKVEIK

SEQ ID NO: 32 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFRYTNYADKVKGRFTISRDNAKNSLYLQMNSLRDEDTAVYFCARYVYQVVAIGDLWGQGTLVTVSS

SEQ ID NO: 33 - protein, 107 aa, Artificial Sequence
  Made in Lab - Synthesized light chain variable region sequence
DIQMTQSPSSVSASVGDRVTITCRASQGISSWLAWYQQKPGRAPKLLIFAASSLQSGVPSRFSGSGSGTHFTLTISSLQPEDFATYYCQQAESFPYTFGQGTKLEIK

SEQ ID NO: 34 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDKSRYTKYTDKVRGRFTISRDNAKNSLYLQMNSLRAEDTAVYYCARYLYQVIAIADAWGQGTLVTVSS

SEQ ID NO: 35 - protein, 107 aa, Artificial Sequence
  Made in Lab - Synthesized light chain variable region sequence
DIQMTQSPSSVSASVGDRVTITCRASQGISSWLAWFQQKPGKAPKLLIFVASNLESGVPSRFSGSGSGTDFTLTISSLQPEDFATYYCQQAESFPYTFGQGTKLEIK

SEQ ID NO: 36 - protein, 10 aa, Artificial Sequence
  Made in Lab - Synthesized CDR sequence
GFTFSDYYMT

SEQ ID NO: 37 - protein, 17 aa, Artificial Sequence
  Made in Lab - Synthesized CDR sequence
YISGSNAYTDYADSVKG

SEQ ID NO: 38 - protein, 11 aa, Artificial Sequence
  Made in Lab - Synthesized CDR sequence
YVYQVVAIGDY

SEQ ID NO: 39 - protein, 11 aa, Artificial Sequence
  Made in Lab - Synthesized CDR sequence
RASQGISSWLA

SEQ ID NO: 40 - protein, 7 aa, Artificial Sequence
  Made in Lab - Synthesized CDR sequence
AASSLQS

SEQ ID NO: 41 - protein, 9 aa, Artificial Sequence
  Made in Lab - Synthesized CDR sequence
QQAKSFPYT

SEQ ID NO: 42 - protein, 10 aa, Artificial Sequence
  Made in Lab - Synthesized CDR sequence
GFTFSDYYMT

SEQ ID NO: 43 - protein, 17 aa, Artificial Sequence
  Made in Lab - Synthesized CDR sequence
YISGSNAYTDYADSVKG

SEQ ID NO: 44 - protein, 11 aa, Artificial Sequence
  Made in Lab - Synthesized CDR sequence
YVYQTVAIGDL

SEQ ID NO: 45 - protein, 11 aa, Artificial Sequence
  Made in Lab - Synthesized CDR sequence
RASQGISSWLA

SEQ ID NO: 46 - protein, 7 aa, Artificial Sequence
  Made in Lab - Synthesized CDR sequence
AASSLQS

SEQ ID NO: 47 - protein, 9 aa, Artificial Sequence
  Made in Lab - Synthesized CDR sequence
QQAKSFPYT

SEQ ID NO: 48 - protein, 10 aa, Artificial Sequence
  Made in Lab - Synthesized CDR sequence
GFTFSDYYMT

SEQ ID NO: 49 - protein, 17 aa, Artificial Sequence
  Made in Lab - Synthesized CDR sequence
YISGSNAYTDYADSVKG

SEQ ID NO: 50 - protein, 11 aa, Artificial Sequence
  Made in Lab - Synthesized CDR sequence
YVYQVVAVGDL

SEQ ID NO: 51 - protein, 11 aa, Artificial Sequence
  Made in Lab - Synthesized CDR sequence
RASQGISSWLA

SEQ ID NO: 52 - protein, 7 aa, Artificial Sequence
  Made in Lab - Synthesized CDR sequence
AASSLQS

SEQ ID NO: 53 - protein, 9 aa, Artificial Sequence
  Made in Lab - Synthesized CDR sequence
QQAKSFPYT

SEQ ID NO: 54 - protein, 10 aa, Artificial Sequence
  Made in Lab - Synthesized CDR sequence
GFTFSDYYMT

SEQ ID NO: 55 - protein, 17 aa, Artificial Sequence
  Made in Lab - Synthesized CDR sequence
YISGSNAYTDYADSVKG

SEQ ID NO: 56 - protein, 11 aa, Artificial Sequence
  Made in Lab - Synthesized CDR sequence
YVYQVVAIGDL

SEQ ID NO: 57 - protein, 11 aa, Artificial Sequence
  Made in Lab - Synthesized CDR sequence
RASQGISSWLA

SEQ ID NO: 58 - protein, 7 aa, Artificial Sequence
  Made in Lab - Synthesized CDR sequence
AASSLQS

SEQ ID NO: 59 - protein, 9 aa, Artificial Sequence
  Made in Lab - Synthesized CDR sequence
QQAKSFPYT

SEQ ID NO: 60 - protein, 10 aa, Artificial Sequence
  Made in Lab - Synthesized CDR sequence
GFTFSDYYMT

SEQ ID NO: 61 - protein, 17 aa, Artificial Sequence
  Made in Lab - Synthesized CDR sequence
YISGSFAYTDYADSVKG

SEQ ID NO: 62 - protein, 11 aa, Artificial Sequence
  Made in Lab - Synthesized CDR sequence
YVYQVVAIGDY

SEQ ID NO: 63 - protein, 11 aa, Artificial Sequence
  Made in Lab - Synthesized CDR sequence
RASQGISSWLA

SEQ ID NO: 64 - protein, 7 aa, Artificial Sequence
  Made in Lab - Synthesized CDR sequence
AASSLQS

SEQ ID NO: 65 - protein, 9 aa, Artificial Sequence
  Made in Lab - Synthesized CDR sequence
QQAKSFPYT

SEQ ID NO: 66 - protein, 10 aa, Artificial Sequence
  Made in Lab - Synthesized CDR sequence
GFTFSDYYMT

SEQ ID NO: 67 - protein, 17 aa, Artificial Sequence
  Made in Lab - Synthesized CDR sequence
YISGSNAYTNYADSVKG

SEQ ID NO: 68 - protein, 11 aa, Artificial Sequence
  Made in Lab - Synthesized CDR sequence
YVYQVVAIGDY

SEQ ID NO: 69 - protein, 11 aa, Artificial Sequence
  Made in Lab - Synthesized CDR sequence
RASQGISSWLA

SEQ ID NO: 70 - protein, 7 aa, Artificial Sequence
  Made in Lab - Synthesized CDR sequence
AASSLQS

SEQ ID NO: 71 - protein, 9 aa, Artificial Sequence
  Made in Lab - Synthesized CDR sequence
QQAKSFPYT

SEQ ID NO: 72 - protein, 10 aa, Artificial Sequence
  Made in Lab - Synthesized CDR sequence
GFTFSDYYMT

SEQ ID NO: 73 - protein, 17 aa, Artificial Sequence
  Made in Lab - Synthesized CDR sequence
YISGSFAYTNYADSVKG

SEQ ID NO: 74 - protein, 11 aa, Artificial Sequence
  Made in Lab - Synthesized CDR sequence
YVYQVVAIGDY

SEQ ID NO: 75 - protein, 11 aa, Artificial Sequence
  Made in Lab - Synthesized CDR sequence
RASQGISSWLA

SEQ ID NO: 76 - protein, 7 aa, Artificial Sequence
  Made in Lab - Synthesized CDR sequence
AASSLQS

SEQ ID NO: 77 - protein, 9 aa, Artificial Sequence
  Made in Lab - Synthesized CDR sequence
QQAKSFPYT

SEQ ID NO: 78 - protein, 10 aa, Artificial Sequence
  Made in Lab - Synthesized CDR sequence
GFTFSDYYMT

SEQ ID NO: 79 - protein, 17 aa, Artificial Sequence
  Made in Lab - Synthesized CDR sequence
YISGSFRYTNYADSVKG

SEQ ID NO: 80 - protein, 11 aa, Artificial Sequence
  Made in Lab - Synthesized CDR sequence
YVYQVVAIGDY

SEQ ID NO: 81 - protein, 11 aa, Artificial Sequence
  Made in Lab - Synthesized CDR sequence
RASQGISSWLA

SEQ ID NO: 82 - protein, 7 aa, Artificial Sequence
  Made in Lab - Synthesized CDR sequence
AASSLQS

SEQ ID NO: 83 - protein, 9 aa, Artificial Sequence
  Made in Lab - Synthesized CDR sequence
QQAKSFPYT

SEQ ID NO: 84 - protein, 10 aa, Artificial Sequence
  Made in Lab - Synthesized CDR sequence
GFTFSDYYMT

SEQ ID NO: 85 - protein, 17 aa, Artificial Sequence
  Made in Lab - Synthesized CDR sequence
YISGSFRYTNYADSVKG

SEQ ID NO: 86 - protein, 11 aa, Artificial Sequence
  Made in Lab - Synthesized CDR sequence
YVYQVVAIGDL

SEQ ID NO: 87 - protein, 11 aa, Artificial Sequence
  Made in Lab - Synthesized CDR sequence
RASQGISSWLA

SEQ ID NO: 88 - protein, 7 aa, Artificial Sequence
  Made in Lab - Synthesized CDR sequence
AASSLQS

SEQ ID NO: 89 - protein, 9 aa, Artificial Sequence
  Made in Lab - Synthesized CDR sequence
QQAKSFPYT

SEQ ID NO: 90 - protein, 10 aa, Artificial Sequence
  Made in Lab - Synthesized CDR sequence
GFTFSDYYMT

SEQ ID NO: 91 - protein, 17 aa, Artificial Sequence
  Made in Lab - Synthesized CDR sequence
YISGSFRYTNYADSVKG

SEQ ID NO: 92 - protein, 11 aa, Artificial Sequence
  Made in Lab - Synthesized CDR sequence
YVYQVVAIGDL

SEQ ID NO: 93 - protein, 11 aa, Artificial Sequence
  Made in Lab - Synthesized CDR sequence
RASQGISSWLA

SEQ ID NO: 94 - protein, 7 aa, Artificial Sequence
  Made in Lab - Synthesized CDR sequence
AASSLQS

SEQ ID NO: 95 - protein, 9 aa, Artificial Sequence
  Made in Lab - Synthesized CDR sequence
QQAESFPYT

SEQ ID NO: 96 - protein, 10 aa, Artificial Sequence
  Made in Lab - Synthesized CDR sequence
GFTFSDYYMT

SEQ ID NO: 97 - protein, 17 aa, Artificial Sequence
  Made in Lab - Synthesized CDR sequence
YISGSFRYTNYAPSVKG

SEQ ID NO: 98 - protein, 11 aa, Artificial Sequence
  Made in Lab - Synthesized CDR sequence
YVYQVVAIGDL

SEQ ID NO: 99 - protein, 11 aa, Artificial Sequence
  Made in Lab - Synthesized CDR sequence
RASQGISSWLA

SEQ ID NO: 100 - protein, 7 aa, Artificial Sequence
  Made in Lab - Synthesized CDR sequence
AASSLQS

SEQ ID NO: 101 - protein, 9 aa, Artificial Sequence
  Made in Lab - Synthesized CDR sequence
QQAESFPYT

SEQ ID NO: 102 - protein, 10 aa, Artificial Sequence
  Made in Lab - Synthesized CDR sequence
GFTFSDYYMT

SEQ ID NO: 103 - protein, 17 aa, Artificial Sequence
  Made in Lab - Synthesized CDR sequence
YISGSFRYTNYADKVKG

SEQ ID NO: 104 - protein, 11 aa, Artificial Sequence
  Made in Lab - Synthesized CDR sequence
YVYQVVAIGDL

SEQ ID NO: 105 - protein, 11 aa, Artificial Sequence
  Made in Lab - Synthesized CDR sequence
RASQGISSWLA

SEQ ID NO: 106 - protein, 7 aa, Artificial Sequence
  Made in Lab - Synthesized CDR sequence
AASSLQS

SEQ ID NO: 107 - protein, 9 aa, Artificial Sequence
  Made in Lab - Synthesized CDR sequence
QQAESFPYT

SEQ ID NO: 108 - protein, 10 aa, Artificial Sequence
  Made in Lab - Synthesized CDR sequence
GFTFSDYYMT

SEQ ID NO: 109 - protein, 17 aa, Artificial Sequence
  Made in Lab - Synthesized CDR sequence
YISGSFRYTNYADKVKG

SEQ ID NO: 110 - protein, 11 aa, Artificial Sequence
  Made in Lab - Synthesized CDR sequence
YVYQVVAIGDL

SEQ ID NO: 111 - protein, 11 aa, Artificial Sequence
  Made in Lab - Synthesized CDR sequence
RASQGISSWLA

SEQ ID NO: 112 - protein, 7 aa, Artificial Sequence
  Made in Lab - Synthesized CDR sequence
AASSLQS

SEQ ID NO: 113 - protein, 9 aa, Artificial Sequence
  Made in Lab - Synthesized CDR sequence
QQKESFPYT

SEQ ID NO: 114 - protein, 10 aa, Artificial Sequence
  Made in Lab - Synthesized CDR sequence
GFTFSDYYMS

SEQ ID NO: 115 - protein, 17 aa, Artificial Sequence
  Made in Lab - Synthesized CDR sequence
YISDSSTYTNYTDSVRG

SEQ ID NO: 116 - protein, 11 aa, Artificial Sequence
  Made in Lab - Synthesized CDR sequence
YLYQVIAVADS

SEQ ID NO: 117 - protein, 11 aa, Artificial Sequence
  Made in Lab - Synthesized CDR sequence
RASQGISSWLA

SEQ ID NO: 118 - protein, 7 aa, Artificial Sequence
  Made in Lab - Synthesized CDR sequence
VASNLES

SEQ ID NO: 119 - protein, 9 aa, Artificial Sequence
  Made in Lab - Synthesized CDR sequence
QQANSFPYT

SEQ ID NO: 120 - protein, 10 aa, Artificial Sequence
  Made in Lab - Synthesized CDR sequence
GFTFSDYYMS

SEQ ID NO: 121 - protein, 17 aa, Artificial Sequence
  Made in Lab - Synthesized CDR sequence
YISDSSRYTKYTDSVRG

SEQ ID NO: 122 - protein, 11 aa, Artificial Sequence
  Made in Lab - Synthesized CDR sequence
YLYQVIAIAKS

SEQ ID NO: 123 - protein, 11 aa, Artificial Sequence
  Made in Lab - Synthesized CDR sequence
RASQGISSWLA

SEQ ID NO: 124 - protein, 7 aa, Artificial Sequence
  Made in Lab - Synthesized CDR sequence
VASNLES

SEQ ID NO: 125 - protein, 9 aa, Artificial Sequence
  Made in Lab - Synthesized CDR sequence
QQANSFPYT

SEQ ID NO: 126 - protein, 10 aa, Artificial Sequence
  Made in Lab - Synthesized CDR sequence
GFTFSDYYMS

SEQ ID NO: 127 - protein, 17 aa, Artificial Sequence
  Made in Lab - Synthesized CDR sequence
YISDSSRYTNYADSVRG

SEQ ID NO: 128 - protein, 11 aa, Artificial Sequence
  Made in Lab - Synthesized CDR sequence
YLYQVIAIAKS

SEQ ID NO: 129 - protein, 11 aa, Artificial Sequence
  Made in Lab - Synthesized CDR sequence
RASQGISSWLA

SEQ ID NO: 130 - protein, 7 aa, Artificial Sequence
  Made in Lab - Synthesized CDR sequence
VASNLES

SEQ ID NO: 131 - protein, 9 aa, Artificial Sequence
  Made in Lab - Synthesized CDR sequence
QQANSFPYT

SEQ ID NO: 132 - protein, 10 aa, Artificial Sequence
  Made in Lab - Synthesized CDR sequence
GFTFSDYYMS

SEQ ID NO: 133 - protein, 17 aa, Artificial Sequence
  Made in Lab - Synthesized CDR sequence
YISDSSRYTKYTDSVRG

SEQ ID NO: 134 - protein, 11 aa, Artificial Sequence
  Made in Lab - Synthesized CDR sequence
YLYQVIAIAKS

SEQ ID NO: 135 - protein, 11 aa, Artificial Sequence
  Made in Lab - Synthesized CDR sequence
RASQGISSWLA

SEQ ID NO: 136 - protein, 7 aa, Artificial Sequence
  Made in Lab - Synthesized CDR sequence
VASNLES

SEQ ID NO: 137 - protein, 9 aa, Artificial Sequence
  Made in Lab - Synthesized CDR sequence
QQANSFPYT

SEQ ID NO: 138 - protein, 10 aa, Artificial Sequence
  Made in Lab - Synthesized CDR sequence
GFTFSDYYMS

SEQ ID NO: 139 - protein, 17 aa, Artificial Sequence
  Made in Lab - Synthesized CDR sequence
YISDSSRYTKYTDSVRG

SEQ ID NO: 140 - protein, 11 aa, Artificial Sequence
  Made in Lab - Synthesized CDR sequence
YLYQVIAIAKS

SEQ ID NO: 141 - protein, 11 aa, Artificial Sequence
  Made in Lab - Synthesized CDR sequence
RASQGISSWLA

SEQ ID NO: 142 - protein, 7 aa, Artificial Sequence
  Made in Lab - Synthesized CDR sequence
VASNLES

SEQ ID NO: 143 - protein, 9 aa, Artificial Sequence
  Made in Lab - Synthesized CDR sequence
QQAESFPYT

SEQ ID NO: 144 - protein, 10 aa, Artificial Sequence
  Made in Lab - Synthesized CDR sequence
GFTFSDYYMS

SEQ ID NO: 145 - protein, 17 aa, Artificial Sequence
  Made in Lab - Synthesized CDR sequence
YISDKSRYTKYTPSVRG

SEQ ID NO: 146 - protein, 11 aa, Artificial Sequence
  Made in Lab - Synthesized CDR sequence
YLYQVIAIADA

SEQ ID NO: 147 - protein, 11 aa, Artificial Sequence
  Made in Lab - Synthesized CDR sequence
RASQGISSWLA

SEQ ID NO: 148 - protein, 7 aa, Artificial Sequence
  Made in Lab - Synthesized CDR sequence
VASNLES

SEQ ID NO: 149 - protein, 9 aa, Artificial Sequence
  Made in Lab - Synthesized CDR sequence
QQAESFPYT

SEQ ID NO: 150 - protein, 10 aa, Artificial Sequence
  Made in Lab - Synthesized CDR sequence
GFTFSDYYMS

SEQ ID NO: 151 - protein, 17 aa, Artificial Sequence
  Made in Lab - Synthesized CDR sequence
YISDKSRYTKYTDKVRG

SEQ ID NO: 152 - protein, 11 aa, Artificial Sequence
  Made in Lab - Synthesized CDR sequence
YLYQVIAIADA

SEQ ID NO: 153 - protein, 11 aa, Artificial Sequence
  Made in Lab - Synthesized CDR sequence
RASQGISSWLA

SEQ ID NO: 154 - protein, 7 aa, Artificial Sequence
  Made in Lab - Synthesized CDR sequence
VASNLES

SEQ ID NO: 155 - protein, 9 aa, Artificial Sequence
  Made in Lab - Synthesized CDR sequence
QQAESFPYT

SEQ ID NO: 156 - protein, 10 aa, Artificial Sequence
  Made in Lab - Synthesized CDR sequence
GFTFSDYYMS

SEQ ID NO: 157 - protein, 17 aa, Artificial Sequence
  Made in Lab - Synthesized CDR sequence
YISDKSRYTKYTDSVRG

SEQ ID NO: 158 - protein, 11 aa, Artificial Sequence
  Made in Lab - Synthesized CDR sequence
YLYQVIAIADA

SEQ ID NO: 159 - protein, 11 aa, Artificial Sequence
  Made in Lab - Synthesized CDR sequence
RASQGISSWLA

SEQ ID NO: 160 - protein, 7 aa, Artificial Sequence
  Made in Lab - Synthesized CDR sequence
VASNLES

SEQ ID NO: 161 - protein, 9 aa, Artificial Sequence
  Made in Lab - Synthesized CDR sequence
QQAESFPYT

SEQ ID NO: 162 - protein, 10 aa, Artificial Sequence
  Made in Lab - Synthesized CDR sequence
GFTFSDYYMS

SEQ ID NO: 163 - protein, 17 aa, Artificial Sequence
  Made in Lab - Synthesized CDR sequence
YISDSSRYTKYTDKVRG

SEQ ID NO: 164 - protein, 11 aa, Artificial Sequence
  Made in Lab - Synthesized CDR sequence
YLYQVIAIADA

SEQ ID NO: 165 - protein, 11 aa, Artificial Sequence
  Made in Lab - Synthesized CDR sequence
RASQGISSWLA

SEQ ID NO: 166 - protein, 7 aa, Artificial Sequence
  Made in Lab - Synthesized CDR sequence
VASNLES

SEQ ID NO: 167 - protein, 9 aa, Artificial Sequence
  Made in Lab - Synthesized CDR sequence
QQAESFPYT

SEQ ID NO: 168 - protein, 10 aa, Artificial Sequence
  Made in Lab - Synthesized CDR sequence
GYTFTDYAIG

SEQ ID NO: 169 - protein, 17 aa, Artificial Sequence
  Made in Lab - Synthesized CDR sequence
DICPGDAYTNDNEKFKD

SEQ ID NO: 170 - protein, 13 aa, Artificial Sequence
  Made in Lab - Synthesized CDR sequence
GEEQVGLRNAMDY

SEQ ID NO: 171 - protein, 14 aa, Artificial Sequence
  Made in Lab - Synthesized CDR sequence
QSQSVSTSTYNYMH

SEQ ID NO: 172 - protein, 7 aa, Artificial Sequence
  Made in Lab - Synthesized CDR sequence
YASNLES

SEQ ID NO: 173 - protein, 9 aa, Artificial Sequence
  Made in Lab - Synthesized CDR sequence
QHSWEIPWT

SEQ ID NO: 174 - protein, 10 aa, Artificial Sequence
  Made in Lab - Synthesized CDR sequence
GYTFTDYCIG

SEQ ID NO: 175 - protein, 17 aa, Artificial Sequence
  Made in Lab - Synthesized CDR sequence
DICPGDAYTNDNEKFKD

SEQ ID NO: 176 - protein, 13 aa, Artificial Sequence
  Made in Lab - Synthesized CDR sequence
GEEQVGLRNAMDY

SEQ ID NO: 177 - protein, 14 aa, Artificial Sequence
  Made in Lab - Synthesized CDR sequence
QSQSVSTSTYNYMH

SEQ ID NO: 178 - protein, 7 aa, Artificial Sequence
  Made in Lab - Synthesized CDR sequence
YASNLES

SEQ ID NO: 179 - protein, 9 aa, Artificial Sequence
  Made in Lab - Synthesized CDR sequence
QHSWEIPWT

SEQ ID NO: 180 - protein, 10 aa, Artificial Sequence
  Made in Lab - Synthesized CDR sequence
GYTFTDYSIG

SEQ ID NO: 181 - protein, 17 aa, Artificial Sequence
  Made in Lab - Synthesized CDR sequence
DICPGDAYTNDNEKFKD

SEQ ID NO: 182 - protein, 13 aa, Artificial Sequence
  Made in Lab - Synthesized CDR sequence
GEEQVGLRNAMDY

SEQ ID NO: 183 - protein, 14 aa, Artificial Sequence
  Made in Lab - Synthesized CDR sequence
QSQSVSTSTYNYMH

SEQ ID NO: 184 - protein, 7 aa, Artificial Sequence
  Made in Lab - Synthesized CDR sequence
YASNLES

SEQ ID NO: 185 - protein, 9 aa, Artificial Sequence
  Made in Lab - Synthesized CDR sequence
QHSWEIPWT

SEQ ID NO: 186 - protein, 10 aa, Artificial Sequence
  Made in Lab - Synthesized CDR sequence
GYTFTDYCIG

SEQ ID NO: 187 - protein, 17 aa, Artificial Sequence
  Made in Lab - Synthesized CDR sequence
DIAPGDAYTNDNEKFKD

SEQ ID NO: 188 - protein, 13 aa, Artificial Sequence
  Made in Lab - Synthesized CDR sequence
GEEQVGLRNAMDY

SEQ ID NO: 189 - protein, 14 aa, Artificial Sequence
  Made in Lab - Synthesized CDR sequence
QSQSVSTSTYNYMH

SEQ ID NO: 190 - protein, 7 aa, Artificial Sequence
  Made in Lab - Synthesized CDR sequence
YASNLES

SEQ ID NO: 191 - protein, 9 aa, Artificial Sequence
  Made in Lab - Synthesized CDR sequence
QHSWEIPWT

SEQ ID NO: 192 - protein, 10 aa, Artificial Sequence
  Made in Lab - Synthesized CDR sequence
GYTFTDYCIG

SEQ ID NO: 193 - protein, 17 aa, Artificial Sequence
  Made in Lab - Synthesized CDR sequence
DISPGDAYTNDNEKFKD

SEQ ID NO: 194 - protein, 13 aa, Artificial Sequence
  Made in Lab - Synthesized CDR sequence
GEEQVGLRNAMDY

SEQ ID NO: 195 - protein, 14 aa, Artificial Sequence
  Made in Lab - Synthesized CDR sequence
QSQSVSTSTYNYMH

SEQ ID NO: 196 - protein, 7 aa, Artificial Sequence
  Made in Lab - Synthesized CDR sequence
YASNLES

SEQ ID NO: 197 - protein, 9 aa, Artificial Sequence
  Made in Lab - Synthesized CDR sequence
QHSWEIPWT

SEQ ID NO: 198 - protein, 10 aa, Artificial Sequence
  Made in Lab - Synthesized CDR sequence
GYTFTDYCIG

SEQ ID NO: 199 - protein, 17 aa, Artificial Sequence
  Made in Lab - Synthesized CDR sequence
DICPGDAYTNDNEKFKD

SEQ ID NO: 200 - protein, 13 aa, Artificial Sequence
  Made in Lab - Synthesized CDR sequence
GEEQVGLRNAMDY

SEQ ID NO: 201 - protein, 14 aa, Artificial Sequence
  Made in Lab - Synthesized CDR sequence
QSQSVSTSTYNYMH

SEQ ID NO: 202 - protein, 7 aa, Artificial Sequence
  Made in Lab - Synthesized CDR sequence
YASNLES

SEQ ID NO: 203 - protein, 9 aa, Artificial Sequence
  Made in Lab - Synthesized CDR sequence
QHSWEIPWT

SEQ ID NO: 204 - protein, 10 aa, Artificial Sequence
  Made in Lab - Synthesized CDR sequence
GYTFTDYCIG

SEQ ID NO: 205 - protein, 17 aa, Artificial Sequence
  Made in Lab - Synthesized CDR sequence
DICPGDAYTNDNEKFKD

SEQ ID NO: 206 - protein, 13 aa, Artificial Sequence
  Made in Lab - Synthesized CDR sequence
GEEQVGLRNAMDY

SEQ ID NO: 207 - protein, 14 aa, Artificial Sequence
  Made in Lab - Synthesized CDR sequence
QSQSVSTSTYNYMH

SEQ ID NO: 208 - protein, 7 aa, Artificial Sequence
  Made in Lab - Synthesized CDR sequence
YASNLES

SEQ ID NO: 209 - protein, 9 aa, Artificial Sequence
  Made in Lab - Synthesized CDR sequence
QHSWEIPWT

SEQ ID NO: 210 - protein, 10 aa, Artificial Sequence
  Made in Lab - Synthesized CDR sequence
GYTFTDYCIG

SEQ ID NO: 211 - protein, 17 aa, Artificial Sequence
  Made in Lab - Synthesized CDR sequence
DICPGDAYTNDNEKFKD

SEQ ID NO: 212 - protein, 13 aa, Artificial Sequence
  Made in Lab - Synthesized CDR sequence
GEEQVGLRNAMDY

SEQ ID NO: 213 - protein, 14 aa, Artificial Sequence
  Made in Lab - Synthesized CDR sequence
QSQSVSTSTYNYMH

SEQ ID NO: 214 - protein, 7 aa, Artificial Sequence
  Made in Lab - Synthesized CDR sequence
YASNLES

SEQ ID NO: 215 - protein, 9 aa, Artificial Sequence
  Made in Lab - Synthesized CDR sequence
QHSWEIPWT

SEQ ID NO: 216 - protein, 10 aa, Artificial Sequence
  Made in Lab - Synthesized CDR sequence
GYTFTDYCIG

SEQ ID NO: 217 - protein, 17 aa, Artificial Sequence
  Made in Lab - Synthesized CDR sequence
DICPGDAYTNDNEKFKD

SEQ ID NO: 218 - protein, 13 aa, Artificial Sequence
  Made in Lab - Synthesized CDR sequence
GEEQVGLRNAMDY

SEQ ID NO: 219 - protein, 14 aa, Artificial Sequence
  Made in Lab - Synthesized CDR sequence
QSQSVSTSTYNYMH

SEQ ID NO: 220 - protein, 7 aa, Artificial Sequence
  Made in Lab - Synthesized CDR sequence
YASNLES

SEQ ID NO: 221 - protein, 9 aa, Artificial Sequence
  Made in Lab - Synthesized CDR sequence
QHSWEIPWT

SEQ ID NO: 222 - protein, 10 aa, Artificial Sequence
  Made in Lab - Synthesized CDR sequence
GYTFTDYCIG

SEQ ID NO: 223 - protein, 17 aa, Artificial Sequence
  Made in Lab - Synthesized CDR sequence
DICPGDAYTNDNEKFKD

SEQ ID NO: 224 - protein, 13 aa, Artificial Sequence
  Made in Lab - Synthesized CDR sequence
GEEQLGLRNAMDY

SEQ ID NO: 225 - protein, 14 aa, Artificial Sequence
  Made in Lab - Synthesized CDR sequence
QSQSVSTSTYNYMH

SEQ ID NO: 226 - protein, 7 aa, Artificial Sequence
  Made in Lab - Synthesized CDR sequence
YASNLES

SEQ ID NO: 227 - protein, 9 aa, Artificial Sequence
  Made in Lab - Synthesized CDR sequence
QHSWEIPWT

SEQ ID NO: 228 - protein, 10 aa, Artificial Sequence
  Made in Lab - Synthesized CDR sequence
GYTFTDYCIG

SEQ ID NO: 229 - protein, 17 aa, Artificial Sequence
  Made in Lab - Synthesized CDR sequence
DICPGDAYTNDNEKFKD

SEQ ID NO: 230 - protein, 13 aa, Artificial Sequence
  Made in Lab - Synthesized CDR sequence
GEEQVGLRNAMDY

SEQ ID NO: 231 - protein, 14 aa, Artificial Sequence
  Made in Lab - Synthesized CDR sequence
QSQSVSTSTYNYMH

SEQ ID NO: 232 - protein, 7 aa, Artificial Sequence
  Made in Lab - Synthesized CDR sequence
YASNLES

SEQ ID NO: 233 - protein, 9 aa, Artificial Sequence
  Made in Lab - Synthesized CDR sequence
QHSWEIPWT

SEQ ID NO: 234 - protein, 10 aa, Artificial Sequence
  Made in Lab - Synthesized CDR sequence
GYTFTDYCIG

SEQ ID NO: 235 - protein, 17 aa, Artificial Sequence
  Made in Lab - Synthesized CDR sequence
DICPGDAYTNDNEKFKD

SEQ ID NO: 236 - protein, 13 aa, Artificial Sequence
  Made in Lab - Synthesized CDR sequence
GEEQVGLRNAMDY

SEQ ID NO: 237 - protein, 14 aa, Artificial Sequence
  Made in Lab - Synthesized CDR sequence
QSQSVSTSTYNYMH

SEQ ID NO: 238 - protein, 7 aa, Artificial Sequence
  Made in Lab - Synthesized CDR sequence
YASNLES

SEQ ID NO: 239 - protein, 9 aa, Artificial Sequence
  Made in Lab - Synthesized CDR sequence
QHSWEIPWT

SEQ ID NO: 240 - protein, 10 aa, Artificial Sequence
  Made in Lab - Synthesized CDR sequence
GYTFTDYCIG

SEQ ID NO: 241 - protein, 17 aa, Artificial Sequence
  Made in Lab - Synthesized CDR sequence
DICPGDAYTNDNEKFKD

SEQ ID NO: 242 - protein, 13 aa, Artificial Sequence
  Made in Lab - Synthesized CDR sequence
GEEQVGLRNAMDY

SEQ ID NO: 243 - protein, 14 aa, Artificial Sequence
  Made in Lab - Synthesized CDR sequence
QSQSVSTSTYNYMH

SEQ ID NO: 244 - protein, 7 aa, Artificial Sequence
  Made in Lab - Synthesized CDR sequence
YASNLES

SEQ ID NO: 245 - protein, 9 aa, Artificial Sequence
  Made in Lab - Synthesized CDR sequence
QHSWEIPWT

SEQ ID NO: 246 - protein, 10 aa, Artificial Sequence
  Made in Lab - Synthesized CDR sequence
GYTFTDYCIG

SEQ ID NO: 247 - protein, 17 aa, Artificial Sequence
  Made in Lab - Synthesized CDR sequence
DICPGDTYTNDNEKFKD

SEQ ID NO: 248 - protein, 13 aa, Artificial Sequence
  Made in Lab - Synthesized CDR sequence
GEEQLGLRNAMDY

SEQ ID NO: 249 - protein, 14 aa, Artificial Sequence
  Made in Lab - Synthesized CDR sequence
QSQSVSTSTYNYMH

SEQ ID NO: 250 - protein, 7 aa, Artificial Sequence
  Made in Lab - Synthesized CDR sequence
YASNLES

SEQ ID NO: 251 - protein, 9 aa, Artificial Sequence
  Made in Lab - Synthesized CDR sequence
QHSWEIPWT

SEQ ID NO: 252 - protein, 10 aa, Artificial Sequence
  Made in Lab - Synthesized CDR sequence
GYTFTDYCIG

SEQ ID NO: 253 - protein, 17 aa, Artificial Sequence
  Made in Lab - Synthesized CDR sequence
DICPGDVYTNDNEKFKD

SEQ ID NO: 254 - protein, 13 aa, Artificial Sequence
  Made in Lab - Synthesized CDR sequence
GEEQLGLRNAMDY

SEQ ID NO: 255 - protein, 14 aa, Artificial Sequence
  Made in Lab - Synthesized CDR sequence
QSQSVSTSTYNYMH

SEQ ID NO: 256 - protein, 7 aa, Artificial Sequence
  Made in Lab - Synthesized CDR sequence
YASNLES

SEQ ID NO: 257 - protein, 9 aa, Artificial Sequence
  Made in Lab - Synthesized CDR sequence
QHSWEIPWT

SEQ ID NO: 258 - protein, 10 aa, Artificial Sequence
  Made in Lab - Synthesized CDR sequence
GYTFTDYCIG

SEQ ID NO: 259 - protein, 17 aa, Artificial Sequence
  Made in Lab - Synthesized CDR sequence
DICPGDTYTNDNEKFKD

SEQ ID NO: 260 - protein, 13 aa, Artificial Sequence
  Made in Lab - Synthesized CDR sequence
GEEQLGLRNAMDY

SEQ ID NO: 261 - protein, 14 aa, Artificial Sequence
  Made in Lab - Synthesized CDR sequence
QSQSVSTSTYNYMH

SEQ ID NO: 262 - protein, 7 aa, Artificial Sequence
  Made in Lab - Synthesized CDR sequence
YASNLES

SEQ ID NO: 263 - protein, 9 aa, Artificial Sequence
  Made in Lab - Synthesized CDR sequence
QHSWEIPWT

SEQ ID NO: 264 - protein, 10 aa, Artificial Sequence
  Made in Lab - Synthesized CDR sequence
GYTFTDYCIG

SEQ ID NO: 265 - protein, 17 aa, Artificial Sequence
  Made in Lab - Synthesized CDR sequence
DICPGDVYTNDNEKFKD

SEQ ID NO: 266 - protein, 13 aa, Artificial Sequence
  Made in Lab - Synthesized CDR sequence
GEEQLGLRNAMDY

SEQ ID NO: 267 - protein, 14 aa, Artificial Sequence
  Made in Lab - Synthesized CDR sequence
QSQSVSTSTYNYMH

SEQ ID NO: 268 - protein, 7 aa, Artificial Sequence
  Made in Lab - Synthesized CDR sequence
YASNLES

SEQ ID NO: 269 - protein, 9 aa, Artificial Sequence
  Made in Lab - Synthesized CDR sequence
QHSWEIPWT

SEQ ID NO: 270 - protein, 10 aa, Artificial Sequence
  Made in Lab - Synthesized CDR sequence
GYTFTDYCIG

SEQ ID NO: 271 - protein, 17 aa, Artificial Sequence
  Made in Lab - Synthesized CDR sequence
DICPGDTYTNDNEKFKD

SEQ ID NO: 272 - protein, 13 aa, Artificial Sequence
  Made in Lab - Synthesized CDR sequence
GEEQLGLRNAMDY

SEQ ID NO: 273 - protein, 14 aa, Artificial Sequence
  Made in Lab - Synthesized CDR sequence
QSQSVSTSTYNYMH

SEQ ID NO: 274 - protein, 7 aa, Artificial Sequence
  Made in Lab - Synthesized CDR sequence
YASNLES

SEQ ID NO: 275 - protein, 9 aa, Artificial Sequence
  Made in Lab - Synthesized CDR sequence
GHSYEIPWT

SEQ ID NO: 276 - protein, 10 aa, Artificial Sequence
  Made in Lab - Synthesized CDR sequence
GYTFTDYCIG

SEQ ID NO: 277 - protein, 17 aa, Artificial Sequence
  Made in Lab - Synthesized CDR sequence
DICPGDTYTNDNEKFKD

SEQ ID NO: 278 - protein, 13 aa, Artificial Sequence
  Made in Lab - Synthesized CDR sequence
GEEQLGLRNAMDY

SEQ ID NO: 279 - protein, 14 aa, Artificial Sequence
  Made in Lab - Synthesized CDR sequence
QSQSVSTSTYNYMH

SEQ ID NO: 280 - protein, 7 aa, Artificial Sequence
  Made in Lab - Synthesized CDR sequence
YASNLES

SEQ ID NO: 281 - protein, 9 aa, Artificial Sequence
  Made in Lab - Synthesized CDR sequence
QHSWEIPWT

SEQ ID NO: 282 - protein, 10 aa, Artificial Sequence
  Made in Lab - Synthesized CDR sequence
GYTFTDYCIG

SEQ ID NO: 283 - protein, 17 aa, Artificial Sequence
  Made in Lab - Synthesized CDR sequence
DICPGDTYTNDNEKFKD

SEQ ID NO: 284 - protein, 13 aa, Artificial Sequence
  Made in Lab - Synthesized CDR sequence
GEEQLGLRNAMDY

SEQ ID NO: 285 - protein, 14 aa, Artificial Sequence
  Made in Lab - Synthesized CDR sequence
QSQSVSTSTYNYMH

SEQ ID NO: 286 - protein, 7 aa, Artificial Sequence
  Made in Lab - Synthesized CDR sequence
YASNLES

SEQ ID NO: 287 - protein, 9 aa, Artificial Sequence
  Made in Lab - Synthesized CDR sequence
GHSYEIPWT

SEQ ID NO: 288 - protein, 10 aa, Artificial Sequence
  Made in Lab - Synthesized CDR sequence
GYTFTDYCIG

SEQ ID NO: 289 - protein, 17 aa, Artificial Sequence
  Made in Lab - Synthesized CDR sequence
DICPGDTYTNDNEKFKD

SEQ ID NO: 290 - protein, 13 aa, Artificial Sequence
  Made in Lab - Synthesized CDR sequence
GEEQLGLRNAMDY

SEQ ID NO: 291 - protein, 14 aa, Artificial Sequence
  Made in Lab - Synthesized CDR sequence
QSQSVSTSTYNYMH

SEQ ID NO: 292 - protein, 7 aa, Artificial Sequence
  Made in Lab - Synthesized CDR sequence
YASNLES

SEQ ID NO: 293 - protein, 9 aa, Artificial Sequence
  Made in Lab - Synthesized CDR sequence
GHSYEIPWT

SEQ ID NO: 294 - protein, 10 aa, Artificial Sequence
  Made in Lab - Synthesized CDR sequence
GYTFTDYCIG

SEQ ID NO: 295 - protein, 17 aa, Artificial Sequence
  Made in Lab - Synthesized CDR sequence
DICPGDTYTNDNEKFKD

SEQ ID NO: 296 - protein, 13 aa, Artificial Sequence
  Made in Lab - Synthesized CDR sequence
GEEQLGLRNAMDY

SEQ ID NO: 297 - protein, 14 aa, Artificial Sequence
  Made in Lab - Synthesized CDR sequence
QSQSVSTSTYNYMH

SEQ ID NO: 298 - protein, 7 aa, Artificial Sequence
  Made in Lab - Synthesized CDR sequence
YASNLES

SEQ ID NO: 299 - protein, 9 aa, Artificial Sequence
  Made in Lab - Synthesized CDR sequence
GHSYEIPWT

SEQ ID NO: 300 - protein, 10 aa, Artificial Sequence
  Made in Lab - Synthesized CDR sequence
GYTFTDYCIG

SEQ ID NO: 301 - protein, 17 aa, Artificial Sequence
  Made in Lab - Synthesized CDR sequence
DICPGDTYTNDNEKFKD

SEQ ID NO: 302 - protein, 13 aa, Artificial Sequence
  Made in Lab - Synthesized CDR sequence
GEEQLGLRNAMDY

SEQ ID NO: 303 - protein, 14 aa, Artificial Sequence
  Made in Lab - Synthesized CDR sequence
QSQSVSTSTYNYMH

SEQ ID NO: 304 - protein, 7 aa, Artificial Sequence
  Made in Lab - Synthesized CDR sequence
YASNLES

SEQ ID NO: 305 - protein, 9 aa, Artificial Sequence
  Made in Lab - Synthesized CDR sequence
GHSYEIPWT

SEQ ID NO: 306 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSNAYTDYADSVKGRFTISRDNAKNSLYLQMNSLRDEDTAVYFCARYVYQVVAIGDYWGQGTLVTVSS

SEQ ID NO: 307 - protein, 107 aa, Artificial Sequence
  Made in Lab - Synthesized light chain variable region sequence
DIQMTQSPSSVSASVGDRVTITCRASQGISSWLAWYQQKPGRAPKLLIFAASSLQSGVPSRFSGSGSGTHFTLTISSLQPEDFATYYCQQAKSFPYTFGQGTKLEIK

SEQ ID NO: 308 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSNAYTDYADSVKGRFTISRDNAKNSLYLQMNSLRDEDTAVYFCARYVYQTVAIGDLWGQGTLVTVSS

SEQ ID NO: 309 - protein, 107 aa, Artificial Sequence; identical to SEQ ID NO: 307
  Made in Lab - Synthesized light chain variable region sequence

SEQ ID NO: 310 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSNAYTDYADSVKGRFTISRDNAKNSLYLQMNSLRDEDTAVYFCARYVYQVVAVGDLWGQGTLVTVSS

SEQ ID NO: 311 - protein, 107 aa, Artificial Sequence; identical to SEQ ID NO: 307
  Made in Lab - Synthesized light chain variable region sequence

SEQ ID NO: 312 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSNAYTDYADSVKGRFTISRDNAKNSLYLQMNSLRDEDTAVYFCARYVYQVVAIGDLWGQGTLVTVSS

SEQ ID NO: 313 - protein, 107 aa, Artificial Sequence; identical to SEQ ID NO: 307
  Made in Lab - Synthesized light chain variable region sequence

SEQ ID NO: 314 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFAYTDYADSVKGRFTISRDNAKNSLYLQMNSLRDEDTAVYFCARYVYQVVAIGDYWGQGTLVTVSS

SEQ ID NO: 315 - protein, 107 aa, Artificial Sequence; identical to SEQ ID NO: 307
  Made in Lab - Synthesized light chain variable region sequence

SEQ ID NO: 316 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSNAYTNYADSVKGRFTISRDNAKNSLYLQMNSLRDEDTAVYFCARYVYQVVAIGDYWGQGTLVTVSS

SEQ ID NO: 317 - protein, 107 aa, Artificial Sequence; identical to SEQ ID NO: 307
  Made in Lab - Synthesized light chain variable region sequence

SEQ ID NO: 318 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFAYTNYADSVKGRFTISRDNAKNSLYLQMNSLRDEDTAVYFCARYVYQVVAIGDYWGQGTLVTVSS

SEQ ID NO: 319 - protein, 107 aa, Artificial Sequence; identical to SEQ ID NO: 307
  Made in Lab - Synthesized light chain variable region sequence

SEQ ID NO: 320 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFRYTNYADSVKGRFTISRDNAKNSLYLQMNSLRDEDTAVYFCARYVYQVVAIGDYWGQGTLVTVSS

SEQ ID NO: 321 - protein, 107 aa, Artificial Sequence; identical to SEQ ID NO: 307
  Made in Lab - Synthesized light chain variable region sequence

SEQ ID NO: 322 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFRYTNYADSVKGRFTISRDNAKNSLYLQMNSLRDEDTAVYFCARYVYQVVAIGDLWGQGTLVTVSS

SEQ ID NO: 323 - protein, 107 aa, Artificial Sequence; identical to SEQ ID NO: 307
  Made in Lab - Synthesized light chain variable region sequence

SEQ ID NO: 324 - protein, 120 aa, Artificial Sequence; identical to SEQ ID NO: 322
  Made in Lab - Synthesized heavy chain variable region sequence

SEQ ID NO: 325 - protein, 107 aa, Artificial Sequence; identical to SEQ ID NO: 33
  Made in Lab - Synthesized light chain variable region sequence

SEQ ID NO: 326 - protein, 120 aa, Artificial Sequence
  Made in Lab - Synthesized heavy chain variable region sequence
QVQLVESGGGLVKPGGSLRLSCAASGFTFSDYYMTWIRQAPGKGLEWVSYISGSFRYTNYAPSVKGRFTISRDNAKNSLYLQMNSLRDEDTAVYFCARYVYQVVAIGDLWGQGTLVTVSS

SEQ ID NO: 327 - protein, 107 aa, Artificial Sequence; identical to SEQ ID NO: 33
  Made in Lab - Synthesized light chain variable region sequence

SEQ ID NO: 328 - protein, 120 aa, Artificial Sequence; identical to SEQ ID NO: 32
  Made in Lab - Synthesized heavy chain variable region sequence

SEQ ID NO: 329 - protein, 107 aa, Artificial Sequence; identical to SEQ ID NO: 33
  Made in Lab - Synthesized light chain variable region sequence

SEQ ID NO: 330 - protein, 120 aa, Artificial Sequence; identical to SEQ ID NO: 32
  Made in Lab - Synthesized heavy chain variable region sequence

SEQ ID NO: 331 - protein, 107 aa, Artificial Sequence
  Made in Lab - Synthesized light chain variable region sequence
DIQMTQSPSSVSASVGDRVTITCRASQGISSWLAWYQQKPGRAPKLLIFAASSLQSGVPSRFSGSGSGTHFTLTISSLQPEDFATYYCQQKESFPYTFGQGTKLEIK

SEQ ID NO: 332 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDSSTYTNYTDSVRGRFTISRDNAKNSLYLQMNSLRAEDTAVYYCARYLYQVIAVADSWGQGTLVTVSS

SEQ ID NO: 333 - protein, 107 aa, Artificial Sequence
  Made in Lab - Synthesized light chain variable region sequence
DIQMTQSPSSVSASVGDRVTITCRASQGISSWLAWFQQKPGKAPKLLIYVASNLESGVPSRFSGSGSGTDFTLTISSLQPEDFATYYCQQANSFPYTFGQGTKLEIK

SEQ ID NO: 334 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDSSRYTKYTDSVRGRFTISRDNAKNSLYLQMNSLRAEDTAVYYCARYLYQVIAIAKSWGQGTLVTVSS

SEQ ID NO: 335 - protein, 107 aa, Artificial Sequence; identical to SEQ ID NO: 333
  Made in Lab - Synthesized light chain variable region sequence

SEQ ID NO: 336 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDSSRYTNYADSVRGRFTISRDNAKNSLYLQMNSLRAEDTAVYYCARYLYQVIAIAKSWGQGTLVTVSS

SEQ ID NO: 337 - protein, 107 aa, Artificial Sequence; identical to SEQ ID NO: 333
  Made in Lab - Synthesized light chain variable region sequence

SEQ ID NO: 338 - protein, 120 aa, Artificial Sequence; identical to SEQ ID NO: 334
  Made in Lab - Synthesized heavy chain variable region sequence

SEQ ID NO: 339 - protein, 107 aa, Artificial Sequence
  Made in Lab - Synthesized light chain variable region sequence
DIQMTQSPSSVSASVGDRVTITCRASQGISSWLAWFQQKPGKAPKLLIFVASNLESGVPSRFSGSGSGTDFTLTISSLQPEDFATYYCQQANSFPYTFGQGTKLEIK

SEQ ID NO: 340 - protein, 120 aa, Artificial Sequence; identical to SEQ ID NO: 334
  Made in Lab - Synthesized heavy chain variable region sequence

SEQ ID NO: 341 - protein, 107 aa, Artificial Sequence; identical to SEQ ID NO: 35
  Made in Lab - Synthesized light chain variable region sequence

SEQ ID NO: 342 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDKSRYTKYTPSVRGRFTISRDNAKNSLYLQMNSLRAEDTAVYYCARYLYQVIAIADAWGQGTLVTVSS

SEQ ID NO: 343 - protein, 107 aa, Artificial Sequence; identical to SEQ ID NO: 35
  Made in Lab - Synthesized light chain variable region sequence

SEQ ID NO: 344 - protein, 120 aa, Artificial Sequence; identical to SEQ ID NO: 34
  Made in Lab - Synthesized heavy chain variable region sequence

SEQ ID NO: 345 - protein, 107 aa, Artificial Sequence; identical to SEQ ID NO: 35
  Made in Lab - Synthesized light chain variable region sequence

SEQ ID NO: 346 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDKSRYTKYTDSVRGRFTISRDNAKNSLYLQMNSLRAEDTAVYYCARYLYQVIAIADAWGQGTLVTVSS

SEQ ID NO: 347 - protein, 107 aa, Artificial Sequence; identical to SEQ ID NO: 35
  Made in Lab - Synthesized light chain variable region sequence

SEQ ID NO: 348 - protein, 120 aa, Artificial Sequence
  Made in Lab - Synthesized heavy chain variable region sequence
QVQLVESGGGLVKPGGSLRLSCAASGFTFSDYYMSWIRQAPGKGLEWVSYISDSSRYTKYTDKVRGRFTISRDNAKNSLYLQMNSLRAEDTAVYYCARYLYQVIAIADAWGQGTLVTVSS

SEQ ID NO: 349 - protein, 107 aa, Artificial Sequence; identical to SEQ ID NO: 35
  Made in Lab - Synthesized light chain variable region sequence

SEQ ID NO: 350 - protein, 122 aa, Artificial Sequence
  Made in Lab - Synthesized heavy chain variable region sequence
QVQLQQSGAELVRPGSSVRMSCKAVGYTFTDYAIGWIKQRPGHGLEWIGDICPGDAYTNDNEKFKDKATLTADTSSTTAYMQLSSLTSEDSAIYYCARGEEQVGLRNAMDYWGQGTSVTVSS

SEQ ID NO: 351 - protein, 110 aa, Artificial Sequence
  Made in Lab - Synthesized light chain variable region sequence
DIVLTQSPASLAVSLGQRATISCQSQSVSTSTYNYMHWYQQKPGQPPKLLIKYASNLESGVPARFSGSGSGTDFTLNIHPVEEEDTATYYCQHSWEIPWTFGGGTKLEIK

SEQ ID NO: 352 - protein, 122 aa, Artificial Sequence
  Made in Lab - Synthesized heavy chain variable region sequence
QVQLQQSGAELVRPGSSVRMSCKAVGYTFTDYCIGWIKQRPGHGLEWIGDICPGDAYTNDNEKFKDKATLTADTSSTTAYMQLSSLTSEDSAIYYCARGEEQVGLRNAMDYWGQGTSVTVSS

SEQ ID NO: 353 - protein, 110 aa, Artificial Sequence; identical to SEQ ID NO: 351
  Made in Lab - Synthesized light chain variable region sequence

SEQ ID NO: 354 - protein, 122 aa, Artificial Sequence
  Made in Lab - Synthesized heavy chain variable region sequence
QVQLQQSGAELVRPGSSVRMSCKAVGYTFTDYSIGWIKQRPGHGLEWIGDICPGDAYTNDNEKFKDKATLTADTSSTTAYMQLSSLTSEDSAIYYCARGEEQVGLRNAMDYWGQGTSVTVSS

SEQ ID NO: 355 - protein, 110 aa, Artificial Sequence; identical to SEQ ID NO: 351
  Made in Lab - Synthesized light chain variable region sequence

SEQ ID NO: 356 - protein, 122 aa, Artificial Sequence
  Made in Lab - Synthesized heavy chain variable region sequence
QVQLQQSGAELVRPGSSVRMSCKAVGYTFTDYCIGWIKQRPGHGLEWIGDIAPGDAYTNDNEKFKDKATLTADTSSTTAYMQLSSLTSEDSAIYYCARGEEQVGLRNAMDYWGQGTSVTVSS

SEQ ID NO: 357 - protein, 110 aa, Artificial Sequence; identical to SEQ ID NO: 351
  Made in Lab - Synthesized light chain variable region sequence

SEQ ID NO: 358 - protein, 122 aa, Artificial Sequence
  Made in Lab - Synthesized heavy chain variable region sequence
QVQLQQSGAELVRPGSSVRMSCKAVGYTFTDYCIGWIKQRPGHGLEWIGDISPGDAYTNDNEKFKDKATLTADTSSTTAYMQLSSLTSEDSAIYYCARGEEQVGLRNAMDYWGQGTSVTVSS

SEQ ID NO: 359 - protein, 110 aa, Artificial Sequence; identical to SEQ ID NO: 351
  Made in Lab - Synthesized light chain variable region sequence

SEQ ID NO: 360 - protein, 122 aa, Artificial Sequence
  Made in Lab - Synthesized heavy chain variable region sequence
QVQLVQSGAEVKKPGASVKVSCKASGYTFTDYCIGWVRQAPGQGLEWMGDICPGDAYTNDNEKFKDRVTMTRDTSTSTVYMELSSLRSEDTAVYYCARGEEQVGLRNAMDYWGQGTSVTVSS

SEQ ID NO: 361 - protein, 110 aa, Artificial Sequence
  Made in Lab - Synthesized light chain variable region sequence
DIVMTQSPDSLAVSLGERATINCQSQSVSTSTYNYMHWYQQKPGQPPKLLIYYASNLESGVPDRFSGSGSGTDFTLTISSLQAEDVAVYYCQHSWEIPWTFGGGTKLEIK

SEQ ID NO: 362 - protein, 122 aa, Artificial Sequence
  Made in Lab - Synthesized heavy chain variable region sequence
QVQLVQSGAEVKKPGASVKVSCKASGYTFTDYCIGWIRQAPGQGLEWMGDICPGDAYTNDNEKFKDRATMTADTSTSTAYMELSSLRSEDTAVYYCARGEEQVGLRNAMDYWGQGTSVTVSS

SEQ ID NO: 363 - protein, 110 aa, Artificial Sequence
  Made in Lab - Synthesized light chain variable region sequence
DIVMTQSPDSLAVSLGERATINCQSQSVSTSTYNYMHWYQQKPGQPPKLLIKYASNLESGVPDRFSGSGSGTDFTLTISSLQAEDVATYYCQHSWEIPWTFGGGTKLEIK

SEQ ID NO: 364 - protein, 122 aa, Artificial Sequence; identical to SEQ ID NO: 362
  Made in Lab - Synthesized heavy chain variable region sequence

SEQ ID NO: 365 - protein, 110 aa, Artificial Sequence; identical to SEQ ID NO: 361
  Made in Lab - Synthesized light chain variable region sequence

SEQ ID NO: 366 - protein, 122 aa, Artificial Sequence; identical to SEQ ID NO: 360
  Made in Lab - Synthesized heavy chain variable region sequence

SEQ ID NO: 367 - protein, 110 aa, Artificial Sequence; identical to SEQ ID NO: 363
  Made in Lab - Synthesized light chain variable region sequence

SEQ ID NO: 368 - protein, 122 aa, Artificial Sequence
  Made in Lab - Synthesized heavy chain variable region sequence
QVQLVQSGAEVKKPGASVKVSCKASGYTFTDYCIGWIRQAPGQGLEWMGDICPGDAYTNDNEKFKDRATMTADTSTSTAYMELSSLRSEDTAVYYCARGEEQLGLRNAMDYWGQGTSVTVSS

SEQ ID NO: 369 - protein, 110 aa, Artificial Sequence; identical to SEQ ID NO: 363
  Made in Lab - Synthesized light chain variable region sequence

SEQ ID NO: 370 - protein, 122 aa, Artificial Sequence; identical to SEQ ID NO: 352
  Made in Lab - Synthesized heavy chain variable region sequence

SEQ ID NO: 371 - protein, 110 aa, Artificial Sequence; identical to SEQ ID NO: 351
  Made in Lab - Synthesized light chain variable region sequence

SEQ ID NO: 372 - protein, 122 aa, Artificial Sequence; identical to SEQ ID NO: 352
  Made in Lab - Synthesized heavy chain variable region sequence

SEQ ID NO: 373 - protein, 110 aa, Artificial Sequence; identical to SEQ ID NO: 351
  Made in Lab - Synthesized light chain variable region sequence

SEQ ID NO: 374 - protein, 122 aa, Artificial Sequence; identical to SEQ ID NO: 352
  Made in Lab - Synthesized heavy chain variable region sequence

SEQ ID NO: 375 - protein, 110 aa, Artificial Sequence; identical to SEQ ID NO: 351
  Made in Lab - Synthesized light chain variable region sequence

SEQ ID NO: 376 - protein, 122 aa, Artificial Sequence; identical to SEQ ID NO: 30
  Made in Lab - Synthesized heavy chain variable region sequence

SEQ ID NO: 377 - protein, 110 aa, Artificial Sequence
  Made in Lab - Synthesized light chain variable region sequence
DIVMTQSPDSLAVSLGERATINCQSQSVSTSTYNYMHWYQQKPGQPPKLLIKYASNLESGVPDRFSGSGSGTDFTLTISSLQAEDVATYYCQHSWEIPWTFGGGTKVEIK

SEQ ID NO: 378 - protein, 122 aa, Artificial Sequence
  Made in Lab - Synthesized heavy chain variable region sequence
QVQLVQSGAEVKKPGASVKVSCKASGYTFTDYCIGWIRQAPGQGLEWMGDICPGDVYTNDNEKFKDRATMTADTSTSTAYMELSSLRSEDTAVYYCARGEEQLGLRNAMDYWGQGTLVTVSS

SEQ ID NO: 379 - protein, 110 aa, Artificial Sequence; identical to SEQ ID NO: 377
  Made in Lab - Synthesized light chain variable region sequence

SEQ ID NO: 380 - protein, 122 aa, Artificial Sequence; identical to SEQ ID NO: 30
  Made in Lab - Synthesized heavy chain variable region sequence

SEQ ID NO: 381 - protein, 110 aa, Artificial Sequence; identical to SEQ ID NO: 377
  Made in Lab - Synthesized light chain variable region sequence

SEQ ID NO: 382 - protein, 122 aa, Artificial Sequence; identical to SEQ ID NO: 378
  Made in Lab - Synthesized heavy chain variable region sequence

SEQ ID NO: 383 - protein, 110 aa, Artificial Sequence; identical to SEQ ID NO: 377
  Made in Lab - Synthesized light chain variable region sequence

SEQ ID NO: 384 - protein, 122 aa, Artificial Sequence; identical to SEQ ID NO: 30
  Made in Lab - Synthesized heavy chain variable region sequence

SEQ ID NO: 385 - protein, 110 aa, Artificial Sequence
  Made in Lab - Synthesized light chain variable region sequence
DIVMTQSPDSLAVSLGERATINCQSQSVSTSTYNYMHWYQQKPGQPPKLLIKYASNLESGVPDRFSGSGSGTDFTLTISSLQAEDVATYYCGHSYEIPWTFGGGTKVEIK

SEQ ID NO: 386 - protein, 122 aa, Artificial Sequence; identical to SEQ ID NO: 30
  Made in Lab - Synthesized heavy chain variable region sequence

SEQ ID NO: 387 - protein, 110 aa, Artificial Sequence
  Made in Lab - Synthesized light chain variable region sequence
EIVLTQSPATLSLSPGERATLSCQSQSVSTSTYNYMHWYQQKPGQAPRLLIKYASNLESGIPDRFSGSGSGTDFTLTISRLEPEDFATYYCQHSWEIPWTFGGGTKVEIK

SEQ ID NO: 388 - protein, 122 aa, Artificial Sequence; identical to SEQ ID NO: 30
  Made in Lab - Synthesized heavy chain variable region sequence

SEQ ID NO: 389 - protein, 110 aa, Artificial Sequence; identical to SEQ ID NO: 31
  Made in Lab - Synthesized light chain variable region sequence

SEQ ID NO: 390 - protein, 122 aa, Artificial Sequence; identical to SEQ ID NO: 30
  Made in Lab - Synthesized heavy chain variable region sequence

SEQ ID NO: 391 - protein, 110 aa, Artificial Sequence; identical to SEQ ID NO: 31
  Made in Lab - Synthesized light chain variable region sequence

SEQ ID NO: 392 - protein, 122 aa, Artificial Sequence; identical to SEQ ID NO: 30
  Made in Lab - Synthesized heavy chain variable region sequence

SEQ ID NO: 393 - protein, 110 aa, Artificial Sequence; identical to SEQ ID NO: 31
  Made in Lab - Synthesized light chain variable region sequence

SEQ ID NO: 394 - protein, 122 aa, Artificial Sequence; identical to SEQ ID NO: 30
  Made in Lab - Synthesized heavy chain variable region sequence

SEQ ID NO: 395 - protein, 110 aa, Artificial Sequence; identical to SEQ ID NO: 31
  Made in Lab - Synthesized light chain variable region sequence

SEQ ID NO: 396 - protein, 10 aa, Artificial Sequence
  Consensus clone CDR sequence
  MOD_RES (5)..(5): Xaa = T, A, I or S
  MOD_RES (6)..(6): Xaa = D, H, P, Q or Y
  MOD_RES (7)..(7): Xaa = Y or F
  MOD_RES (9)..(9): Xaa = I, L, T or V
  MOD_RES (10)..(10): Xaa = G, F, I, K, L, T or Y
GYTFXXXCXX

SEQ ID NO: 397 - protein, 17 aa, Artificial Sequence
  Consensus clone CDR sequence
  MOD_RES (1)..(1): Xaa = D, G, Y
  MOD_RES (2)..(2): Xaa = I, M, V
  MOD_RES (4)..(4): Xaa = P, L
  MOD_RES (5)..(5): Xaa = G, I, K, R
  MOD_RES (6)..(6): Xaa = D, E, I, V
  MOD_RES (7)..(7): Xaa = A, E, I, P, Q, S, T, V
  MOD_RES (8)..(8): Xaa = Y, E, F, H, K, P, Q, R, S, T, V
  MOD_RES (9)..(9): Xaa = T, A, S
  MOD_RES (10)..(10): Xaa = N, K
  MOD_RES (12)..(12): Xaa = N, T
  MOD_RES (17)..(17): Xaa = D, A, E, V
XXCXXXXXXXDXEKFKX

SEQ ID NO: 398 - protein, 13 aa, Artificial Sequence
  Consensus clone CDR sequence
  MOD_RES (1)..(1): Xaa = G, I, P
  MOD_RES (2)..(2): Xaa = E, Q, V
  MOD_RES (3)..(3): Xaa = E, G
  MOD_RES (4)..(4): Xaa = Q, A, I, T
  MOD_RES (5)..(5): Xaa = V, D, E, I, L, P, T, Y
  MOD_RES (6)..(6): Xaa = G, R, P, T, V
  MOD_RES (8)..(8): Xaa = R, T
  MOD_RES (9)..(9): Xaa = N, P
  MOD_RES (10)..(10): Xaa = A, N, D, Q, H, I, L, K
  MOD_RES (11)..(11): Xaa = M, R
  MOD_RES (12)..(12): Xaa = D, G, H
  MOD_RES (13)..(13): Xaa = Y, A, N, H, I, K, S, T
XXXXXXLXXXXXX

SEQ ID NO: 399 - protein, 14 aa, Artificial Sequence
  Consensus clone CDR sequence
QSQSVSTSTYNYMH

SEQ ID NO: 400 - protein, 7 aa, Artificial Sequence
  Consensus clone CDR sequence
YASNLES

SEQ ID NO: 401 - protein, 9 aa, Artificial Sequence
  Consensus clone CDR sequence
  MOD_RES (1)..(1): Xaa = Q, G
  MOD_RES (2)..(2): Xaa = H, Q, P
  MOD_RES (3)..(3): Xaa = S, A, D, E, F, H, K, L, P, R, Y
  MOD_RES (4)..(4): Xaa = W, D, E, F, P, Y
  MOD_RES (5)..(5): Xaa = E, R, S, T, V
  MOD_RES (6)..(6): Xaa = I, V, L
  MOD_RES (7)..(7): Xaa = P, D, F, K, N, Q, R, Y
  MOD_RES (8)..(8): Xaa = W, D, E, F, Y
  MOD_RES (9)..(9): Xaa = T, N, Q, R
XXXXXXXXX

SEQ ID NO: 402 - protein, 10 aa, Artificial Sequence
  Consensus clone CDR sequence
  MOD_RES (5)..(5): Xaa = S, R, T, N
  MOD_RES (6)..(6): Xaa = D, A, E
  MOD_RES (9)..(9): Xaa = M, V
  MOD_RES (10)..(10): Xaa = T, F, H, K, S, L, V, Y
GFTFXXYYXX

SEQ ID NO: 403 - protein, 17 aa, Artificial Sequence
  Consensus clone CDR sequence
  MOD_RES (2)..(2): Xaa = I, L, T, V
  MOD_RES (5)..(5): Xaa = R, G, S
  MOD_RES (6)..(6): Xaa = N, A, F, G, H, K, L, Q, R, S, T, V, Y
  MOD_RES (7)..(7): Xaa = A, F, H, K, Q, R, S, V, Y
  MOD_RES (9)..(9): Xaa = T, I
  MOD_RES (10)..(10): Xaa = N, S, D
  MOD_RES (11)..(11): Xaa = Y, L, N
  misc_feature (13)..(13): Xaa can be any naturally occurring amino acid
  MOD_RES (14)..(14): Xaa = S, K
YXSGXXXYXXXAXXVKG

SEQ ID NO: 404 - protein, 11 aa, Artificial Sequence
  Consensus clone CDR sequence
  MOD_RES (2)..(2): Xaa = V, Y, L, I, F
  MOD_RES (5)..(5): Xaa = T, V, I, A
  MOD_RES (6)..(6): Xaa = V, L, I
  MOD_RES (7)..(7): Xaa = A, S
  MOD_RES (8)..(8): Xaa = V, Y, T, S, Q, N, L, I, F
  MOD_RES (9)..(9): Xaa = G, V, T, P, N, L, I, A
  MOD_RES (10)..(10): Xaa = D, E
  MOD_RES (11)..(11): Xaa = Y, V, T, S, R, Q, N, L, K, I, H, G, F, E, D, A
YXYQXXXXXXX

SEQ ID NO: 405 - protein, 11 aa, Artificial Sequence
  Consensus clone CDR sequence
RASQGISSWLA

SEQ ID NO: 406 - protein, 7 aa, Artificial Sequence
  Consensus clone CDR sequence
AASSLQS

SEQ ID NO: 407 - protein, 9 aa, Artificial Sequence
  Consensus clone CDR sequence
  MOD_RES (1)..(1): Xaa = S, Q
  MOD_RES (2)..(2): Xaa = Q, E, H, T
  MOD_RES (3)..(3): Xaa = A, I, K, L, S
  MOD_RES (4)..(4): Xaa = K, A, D, E, F, G, H, I, L, N, Q, R, S, T, V
  MOD_RES (5)..(5): Xaa = S, A, E, F, H, K, Q, R
  MOD_RES (7)..(7): Xaa = P, K, R, S, I, L, V, T
  MOD_RES (8)..(8): Xaa = Y, N
  MOD_RES (9)..(9): Xaa = T, A, D, E, F, G, H, I K, L, N, Q, R, S, V, Y
XXXXXFXXX

SEQ ID NO: 408 - protein, 10 aa, Artificial Sequence
  Consensus clone CDR sequence
  MOD_RES (10)..(10): Xaa = S, T
GFTFSDYYMX

SEQ ID NO: 409 - protein, 17 aa, Artificial Sequence
  Consensus clone CDR sequence
  MOD_RES (4)..(4): Xaa = D, E, Y
  MOD_RES (5)..(5): Xaa = S, A, F, H, K, L, P, Q, R, V, Y
  MOD_RES (6)..(6): Xaa = S, N
  MOD_RES (7)..(7): Xaa = T, A, R
  misc_feature (10)..(10): Xaa can be any naturally occurring amino acid
  MOD_RES (12)..(12): Xaa = T, A
  MOD_RES (13)..(13): Xaa = D, A, E, G, H, I, K, L, N, P, Q, R, S, T, V, Y
  MOD_RES (14)..(14): Xaa = S, A, D, F, H, I, K, L, N, P, Q, R, T, V
YISXXXXYTXYXXXVRG

SEQ ID NO: 410 - protein, 11 aa, Artificial Sequence
  Consensus clone CDR sequence
  MOD_RES (1)..(1): Xaa = Y, H, I, F, V
  MOD_RES (2)..(2): Xaa = L, A, I, K, S, T, V
  MOD_RES (3)..(3): Xaa = H, A, F, V, Y
  MOD_RES (4)..(4): Xaa = Q, D, E
  MOD_RES (6)..(6): Xaa = I, R, P, V
  MOD_RES (7)..(7): Xaa = A, I, K, R
  MOD_RES (8)..(8): Xaa = V, I, L, F
  MOD_RES (9)..(9): Xaa = A, F, I, K, P, Q, S, T, V, Y
  MOD_RES (10)..(10): Xaa = D, E, F, G, H, I, K, L, N, R, S, T, V, Y
  MOD_RES (11)..(11): Xaa = S, A, D, E, F, G, H, I, K, L, N, P, Q, R, T, V, Y
XXXXVXXXXXX

SEQ ID NO: 411 - protein, 11 aa, Artificial Sequence
  Consensus clone CDR sequence
RASQGISSWLA

SEQ ID NO: 412 - protein, 7 aa, Artificial Sequence
  Consensus clone CDR sequence
VASNLES

SEQ ID NO: 413 - protein, 9 aa, Artificial Sequence
  Consensus clone CDR sequence
  MOD_RES (4)..(4): Xaa = N, K, E
QQAXSFPYT

SEQ ID NO: 414 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 415 - protein, 327 aa, Artificial Sequence
  Made in Lab - mutated IgG4 sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 416 - protein, 330 aa, Homo sapiens [sequence omitted]

SEQ ID NO: 417 - protein, 107 aa, Homo sapiens [sequence omitted]

SEQ ID NO: 418 - DNA, 24 bp, Artificial Sequence
  Mad in Lab - Primer
tgaggatgacaaagatttgcagct

SEQ ID NO: 419 - DNA, 36 bp, Artificial Sequence
  Mad in Lab - Primer
accgcggccggccgtttatgcctcggagcagcactt

SEQ ID NO: 420 - DNA, 22 bp, Artificial Sequence
  Mad in Lab - Primer
agtgccaagcaagcaactcaaa

SEQ ID NO: 421 - DNA, 36 bp, Artificial Sequence
  Mad in Lab - Primer
aagtgctgctccgaggcataaacggccggccgcggt

SEQ ID NO: 422 - protein, 48 aa, Homo sapiens
MAERAALEELVKLQGERVRGLKQQKASAELIEEEVAKLLKLKAQLGPD

SEQ ID NO: 423 - protein, 80 aa, Homo sapiens
MAERAALEELVKLQGERVRGLKQQKASAELIEEEVAKLLKLKAQLGPDESKQKFVLKTPKGTRDYSPRQMAVREKVFDVI

SEQ ID NO: 424 - protein, 79 aa, Homo sapiens
MAERAALEELVKLQGERVRGLKQQKASAELIEEEVAKLLKLKAQLGPDESKQKFVLKTPKGTRDYSPRQMAVREKVFDV

SEQ ID NO: 425 - protein, 78 aa, Homo sapiens
MAERAALEELVKLQGERVRGLKQQKASAELIEEEVAKLLKLKAQLGPDESKQKFVLKTPKGTRDYSPRQMAVREKVFD

SEQ ID NO: 426 - protein, 77 aa, Homo sapiens
MAERAALEELVKLQGERVRGLKQQKASAELIEEEVAKLLKLKAQLGPDESKQKFVLKTPKGTRDYSPRQMAVREKVF

SEQ ID NO: 427 - protein, 76 aa, Homo sapiens
MAERAALEELVKLQGERVRGLKQQKASAELIEEEVAKLLKLKAQLGPDESKQKFVLKTPKGTRDYSPRQMAVREKV

SEQ ID NO: 428 - protein, 75 aa, Homo sapiens
MAERAALEELVKLQGERVRGLKQQKASAELIEEEVAKLLKLKAQLGPDESKQKFVLKTPKGTRDYSPRQMAVREK

SEQ ID NO: 429 - protein, 74 aa, Homo sapiens
MAERAALEELVKLQGERVRGLKQQKASAELIEEEVAKLLKLKAQLGPDESKQKFVLKTPKGTRDYSPRQMAVRE

SEQ ID NO: 430 - protein, 73 aa, Homo sapiens
MAERAALEELVKLQGERVRGLKQQKASAELIEEEVAKLLKLKAQLGPDESKQKFVLKTPKGTRDYSPRQMAVR

SEQ ID NO: 431 - protein, 72 aa, Homo sapiens
MAERAALEELVKLQGERVRGLKQQKASAELIEEEVAKLLKLKAQLGPDESKQKFVLKTPKGTRDYSPRQMAV

SEQ ID NO: 432 - protein, 71 aa, Homo sapiens
MAERAALEELVKLQGERVRGLKQQKASAELIEEEVAKLLKLKAQLGPDESKQKFVLKTPKGTRDYSPRQMA

SEQ ID NO: 433 - protein, 70 aa, Homo sapiens
MAERAALEELVKLQGERVRGLKQQKASAELIEEEVAKLLKLKAQLGPDESKQKFVLKTPKGTRDYSPRQM

SEQ ID NO: 434 - protein, 69 aa, Homo sapiens
MAERAALEELVKLQGERVRGLKQQKASAELIEEEVAKLLKLKAQLGPDESKQKFVLKTPKGTRDYSPRQ

SEQ ID NO: 435 - protein, 68 aa, Homo sapiens
MAERAALEELVKLQGERVRGLKQQKASAELIEEEVAKLLKLKAQLGPDESKQKFVLKTPKGTRDYSPR

SEQ ID NO: 436 - protein, 67 aa, Homo sapiens
MAERAALEELVKLQGERVRGLKQQKASAELIEEEVAKLLKLKAQLGPDESKQKFVLKTPKGTRDYSP

SEQ ID NO: 437 - protein, 66 aa, Homo sapiens
MAERAALEELVKLQGERVRGLKQQKASAELIEEEVAKLLKLKAQLGPDESKQKFVLKTPKGTRDYS

SEQ ID NO: 438 - protein, 65 aa, Homo sapiens
MAERAALEELVKLQGERVRGLKQQKASAELIEEEVAKLLKLKAQLGPDESKQKFVLKTPKGTRDY

SEQ ID NO: 439 - protein, 64 aa, Homo sapiens
MAERAALEELVKLQGERVRGLKQQKASAELIEEEVAKLLKLKAQLGPDESKQKFVLKTPKGTRD

SEQ ID NO: 440 - protein, 63 aa, Homo sapiens
MAERAALEELVKLQGERVRGLKQQKASAELIEEEVAKLLKLKAQLGPDESKQKFVLKTPKGTR

SEQ ID NO: 441 - protein, 62 aa, Homo sapiens
MAERAALEELVKLQGERVRGLKQQKASAELIEEEVAKLLKLKAQLGPDESKQKFVLKTPKGT

SEQ ID NO: 442 - protein, 61 aa, Homo sapiens
MAERAALEELVKLQGERVRGLKQQKASAELIEEEVAKLLKLKAQLGPDESKQKFVLKTPKG

SEQ ID NO: 443 - protein, 60 aa, Homo sapiens
MAERAALEELVKLQGERVRGLKQQKASAELIEEEVAKLLKLKAQLGPDESKQKFVLKTPK

SEQ ID NO: 444 - protein, 59 aa, Homo sapiens
MAERAALEELVKLQGERVRGLKQQKASAELIEEEVAKLLKLKAQLGPDESKQKFVLKTP

SEQ ID NO: 445 - protein, 58 aa, Homo sapiens
MAERAALEELVKLQGERVRGLKQQKASAELIEEEVAKLLKLKAQLGPDESKQKFVLKT

SEQ ID NO: 446 - protein, 57 aa, Homo sapiens
MAERAALEELVKLQGERVRGLKQQKASAELIEEEVAKLLKLKAQLGPDESKQKFVLK

SEQ ID NO: 447 - protein, 56 aa, Homo sapiens
MAERAALEELVKLQGERVRGLKQQKASAELIEEEVAKLLKLKAQLGPDESKQKFVL

SEQ ID NO: 448 - protein, 55 aa, Homo sapiens
MAERAALEELVKLQGERVRGLKQQKASAELIEEEVAKLLKLKAQLGPDESKQKFV

SEQ ID NO: 449 - protein, 54 aa, Homo sapiens
MAERAALEELVKLQGERVRGLKQQKASAELIEEEVAKLLKLKAQLGPDESKQKF

SEQ ID NO: 450 - protein, 53 aa, Homo sapiens
MAERAALEELVKLQGERVRGLKQQKASAELIEEEVAKLLKLKAQLGPDESKQK

SEQ ID NO: 451 - protein, 52 aa, Homo sapiens
MAERAALEELVKLQGERVRGLKQQKASAELIEEEVAKLLKLKAQLGPDESKQ

SEQ ID NO: 452 - protein, 51 aa, Homo sapiens
MAERAALEELVKLQGERVRGLKQQKASAELIEEEVAKLLKLKAQLGPDESK

SEQ ID NO: 453 - protein, 50 aa, Homo sapiens
MAERAALEELVKLQGERVRGLKQQKASAELIEEEVAKLLKLKAQLGPDES

SEQ ID NO: 454 - protein, 49 aa, Homo sapiens
MAERAALEELVKLQGERVRGLKQQKASAELIEEEVAKLLKLKAQLGPDE

SEQ ID NO: 455 - protein, 48 aa, Homo sapiens; identical to SEQ ID NO: 422

SEQ ID NO: 456 - protein, 47 aa, Homo sapiens
MAERAALEELVKLQGERVRGLKQQKASAELIEEEVAKLLKLKAQLGP

SEQ ID NO: 457 - protein, 46 aa, Homo sapiens
MAERAALEELVKLQGERVRGLKQQKASAELIEEEVAKLLKLKAQLG

SEQ ID NO: 458 - protein, 45 aa, Homo sapiens
MAERAALEELVKLQGERVRGLKQQKASAELIEEEVAKLLKLKAQL

SEQ ID NO: 459 - protein, 44 aa, Homo sapiens
MAERAALEELVKLQGERVRGLKQQKASAELIEEEVAKLLKLKAQ

SEQ ID NO: 460 - protein, 43 aa, Homo sapiens
MAERAALEELVKLQGERVRGLKQQKASAELIEEEVAKLLKLKA

SEQ ID NO: 461 - protein, 42 aa, Homo sapiens
MAERAALEELVKLQGERVRGLKQQKASAELIEEEVAKLLKLK

SEQ ID NO: 462 - protein, 41 aa, Homo sapiens
MAERAALEELVKLQGERVRGLKQQKASAELIEEEVAKLLKL

SEQ ID NO: 463 - protein, 40 aa, Homo sapiens
MAERAALEELVKLQGERVRGLKQQKASAELIEEEVAKLLK

SEQ ID NO: 464 - protein, 39 aa, Homo sapiens
MAERAALEELVKLQGERVRGLKQQKASAELIEEEVAKLL

SEQ ID NO: 465 - protein, 38 aa, Homo sapiens
MAERAALEELVKLQGERVRGLKQQKASAELIEEEVAKL

SEQ ID NO: 466 - protein, 37 aa, Homo sapiens
MAERAALEELVKLQGERVRGLKQQKASAELIEEEVAK

SEQ ID NO: 467 - protein, 36 aa, Homo sapiens
MAERAALEELVKLQGERVRGLKQQKASAELIEEEVA

SEQ ID NO: 468 - protein, 35 aa, Homo sapiens
MAERAALEELVKLQGERVRGLKQQKASAELIEEEV

SEQ ID NO: 469 - protein, 34 aa, Homo sapiens
MAERAALEELVKLQGERVRGLKQQKASAELIEEE

SEQ ID NO: 470 - protein, 33 aa, Homo sapiens
MAERAALEELVKLQGERVRGLKQQKASAELIEE

SEQ ID NO: 471 - protein, 32 aa, Homo sapiens
MAERAALEELVKLQGERVRGLKQQKASAELIE

SEQ ID NO: 472 - protein, 31 aa, Homo sapiens
MAERAALEELVKLQGERVRGLKQQKASAELI

SEQ ID NO: 473 - protein, 30 aa, Homo sapiens
MAERAALEELVKLQGERVRGLKQQKASAEL

SEQ ID NO: 474 - protein, 79 aa, Homo sapiens
AERAALEELVKLQGERVRGLKQQKASAELIEEEVAKLLKLKAQLGPDESKQKFVLKTPKGTRDYSPRQMAVREKVFDVI

SEQ ID NO: 475 - protein, 78 aa, Homo sapiens
ERAALEELVKLQGERVRGLKQQKASAELIEEEVAKLLKLKAQLGPDESKQKFVLKTPKGTRDYSPRQMAVREKVFDVI

SEQ ID NO: 476 - protein, 77 aa, Homo sapiens
RAALEELVKLQGERVRGLKQQKASAELIEEEVAKLLKLKAQLGPDESKQKFVLKTPKGTRDYSPRQMAVREKVFDVI

SEQ ID NO: 477 - protein, 76 aa, Homo sapiens
AALEELVKLQGERVRGLKQQKASAELIEEEVAKLLKLKAQLGPDESKQKFVLKTPKGTRDYSPRQMAVREKVFDVI

SEQ ID NO: 478 - protein, 75 aa, Homo sapiens
ALEELVKLQGERVRGLKQQKASAELIEEEVAKLLKLKAQLGPDESKQKFVLKTPKGTRDYSPRQMAVREKVFDVI

SEQ ID NO: 479 - protein, 74 aa, Homo sapiens
LEELVKLQGERVRGLKQQKASAELIEEEVAKLLKLKAQLGPDESKQKFVLKTPKGTRDYSPRQMAVREKVFDVI

SEQ ID NO: 480 - protein, 73 aa, Homo sapiens
EELVKLQGERVRGLKQQKASAELIEEEVAKLLKLKAQLGPDESKQKFVLKTPKGTRDYSPRQMAVREKVFDVI

SEQ ID NO: 481 - protein, 72 aa, Homo sapiens
ELVKLQGERVRGLKQQKASAELIEEEVAKLLKLKAQLGPDESKQKFVLKTPKGTRDYSPRQMAVREKVFDVI

SEQ ID NO: 482 - protein, 71 aa, Homo sapiens
LVKLQGERVRGLKQQKASAELIEEEVAKLLKLKAQLGPDESKQKFVLKTPKGTRDYSPRQMAVREKVFDVI

SEQ ID NO: 483 - protein, 70 aa, Homo sapiens
VKLQGERVRGLKQQKASAELIEEEVAKLLKLKAQLGPDESKQKFVLKTPKGTRDYSPRQMAVREKVFDVI

SEQ ID NO: 484 - protein, 69 aa, Homo sapiens
KLQGERVRGLKQQKASAELIEEEVAKLLKLKAQLGPDESKQKFVLKTPKGTRDYSPRQMAVREKVFDVI

SEQ ID NO: 485 - protein, 68 aa, Homo sapiens
LQGERVRGLKQQKASAELIEEEVAKLLKLKAQLGPDESKQKFVLKTPKGTRDYSPRQMAVREKVFDVI

SEQ ID NO: 486 - protein, 67 aa, Homo sapiens
QGERVRGLKQQKASAELIEEEVAKLLKLKAQLGPDESKQKFVLKTPKGTRDYSPRQMAVREKVFDVI

SEQ ID NO: 487 - protein, 66 aa, Homo sapiens
GERVRGLKQQKASAELIEEEVAKLLKLKAQLGPDESKQKFVLKTPKGTRDYSPRQMAVREKVFDVI

SEQ ID NO: 488 - protein, 64 aa, Homo sapiens
RVRGLKQQKASAELIEEEVAKLLKLKAQLGPDESKQKFVLKTPKGTRDYSPRQMAVREKVFDVI

SEQ ID NO: 489 - protein, 63 aa, Homo sapiens
VRGLKQQKASAELIEEEVAKLLKLKAQLGPDESKQKFVLKTPKGTRDYSPRQMAVREKVFDVI

SEQ ID NO: 490 - protein, 62 aa, Homo sapiens
RGLKQQKASAELIEEEVAKLLKLKAQLGPDESKQKFVLKTPKGTRDYSPRQMAVREKVFDVI

SEQ ID NO: 491 - protein, 61 aa, Homo sapiens
GLKQQKASAELIEEEVAKLLKLKAQLGPDESKQKFVLKTPKGTRDYSPRQMAVREKVFDVI

SEQ ID NO: 492 - protein, 60 aa, Homo sapiens
LKQQKASAELIEEEVAKLLKLKAQLGPDESKQKFVLKTPKGTRDYSPRQMAVREKVFDVI

SEQ ID NO: 493 - protein, 59 aa, Homo sapiens
KQQKASAELIEEEVAKLLKLKAQLGPDESKQKFVLKTPKGTRDYSPRQMAVREKVFDVI

SEQ ID NO: 494 - protein, 58 aa, Homo sapiens
QQKASAELIEEEVAKLLKLKAQLGPDESKQKFVLKTPKGTRDYSPRQMAVREKVFDVI

SEQ ID NO: 495 - protein, 57 aa, Homo sapiens
QKASAELIEEEVAKLLKLKAQLGPDESKQKFVLKTPKGTRDYSPRQMAVREKVFDVI

SEQ ID NO: 496 - protein, 56 aa, Homo sapiens
KASAELIEEEVAKLLKLKAQLGPDESKQKFVLKTPKGTRDYSPRQMAVREKVFDVI

SEQ ID NO: 497 - protein, 55 aa, Homo sapiens
ASAELIEEEVAKLLKLKAQLGPDESKQKFVLKTPKGTRDYSPRQMAVREKVFDVI

SEQ ID NO: 498 - protein, 54 aa, Homo sapiens
SAELIEEEVAKLLKLKAQLGPDESKQKFVLKTPKGTRDYSPRQMAVREKVFDVI

SEQ ID NO: 499 - protein, 53 aa, Homo sapiens
AELIEEEVAKLLKLKAQLGPDESKQKFVLKTPKGTRDYSPRQMAVREKVFDVI

SEQ ID NO: 500 - protein, 52 aa, Homo sapiens
ELIEEEVAKLLKLKAQLGPDESKQKFVLKTPKGTRDYSPRQMAVREKVFDVI

SEQ ID NO: 501 - protein, 51 aa, Homo sapiens
LIEEEVAKLLKLKAQLGPDESKQKFVLKTPKGTRDYSPRQMAVREKVFDVI

SEQ ID NO: 502 - protein, 50 aa, Homo sapiens
IEEEVAKLLKLKAQLGPDESKQKFVLKTPKGTRDYSPRQMAVREKVFDVI

SEQ ID NO: 503 - protein, 49 aa, Homo sapiens
EEEVAKLLKLKAQLGPDESKQKFVLKTPKGTRDYSPRQMAVREKVFDVI

SEQ ID NO: 504 - protein, 48 aa, Homo sapiens
EEVAKLLKLKAQLGPDESKQKFVLKTPKGTRDYSPRQMAVREKVFDVI

SEQ ID NO: 505 - protein, 47 aa, Homo sapiens
EVAKLLKLKAQLGPDESKQKFVLKTPKGTRDYSPRQMAVREKVFDVI

SEQ ID NO: 506 - protein, 46 aa, Homo sapiens
VAKLLKLKAQLGPDESKQKFVLKTPKGTRDYSPRQMAVREKVFDVI

SEQ ID NO: 507 - protein, 45 aa, Homo sapiens
AKLLKLKAQLGPDESKQKFVLKTPKGTRDYSPRQMAVREKVFDVI

SEQ ID NO: 508 - protein, 44 aa, Homo sapiens
KLLKLKAQLGPDESKQKFVLKTPKGTRDYSPRQMAVREKVFDVI

SEQ ID NO: 509 - protein, 43 aa, Homo sapiens
LLKLKAQLGPDESKQKFVLKTPKGTRDYSPRQMAVREKVFDVI

SEQ ID NO: 510 - protein, 42 aa, Homo sapiens
LKLKAQLGPDESKQKFVLKTPKGTRDYSPRQMAVREKVFDVI

SEQ ID NO: 511 - protein, 41 aa, Homo sapiens
KLKAQLGPDESKQKFVLKTPKGTRDYSPRQMAVREKVFDVI

SEQ ID NO: 512 - protein, 40 aa, Homo sapiens
LKAQLGPDESKQKFVLKTPKGTRDYSPRQMAVREKVFDVI

SEQ ID NO: 513 - protein, 51 aa, Homo sapiens
LVKLQGERVRGLKQQKASAELIEEEVAKLLKLKAQLGPDESKQKFVLKTPK

SEQ ID NO: 514 - protein, 50 aa, Homo sapiens
VKLQGERVRGLKQQKASAELIEEEVAKLLKLKAQLGPDESKQKFVLKTPK

SEQ ID NO: 515 - protein, 49 aa, Homo sapiens
KLQGERVRGLKQQKASAELIEEEVAKLLKLKAQLGPDESKQKFVLKTPK

SEQ ID NO: 516 - protein, 48 aa, Homo sapiens
LQGERVRGLKQQKASAELIEEEVAKLLKLKAQLGPDESKQKFVLKTPK

SEQ ID NO: 517 - protein, 47 aa, Homo sapiens
QGERVRGLKQQKASAELIEEEVAKLLKLKAQLGPDESKQKFVLKTPK

SEQ ID NO: 518 - protein, 46 aa, Homo sapiens
GERVRGLKQQKASAELIEEEVAKLLKLKAQLGPDESKQKFVLKTPK

SEQ ID NO: 519 - protein, 45 aa, Homo sapiens
ERVRGLKQQKASAELIEEEVAKLLKLKAQLGPDESKQKFVLKTPK

SEQ ID NO: 520 - protein, 44 aa, Homo sapiens
RVRGLKQQKASAELIEEEVAKLLKLKAQLGPDESKQKFVLKTPK

SEQ ID NO: 521 - protein, 43 aa, Homo sapiens
VRGLKQQKASAELIEEEVAKLLKLKAQLGPDESKQKFVLKTPK

SEQ ID NO: 522 - protein, 42 aa, Homo sapiens
RGLKQQKASAELIEEEVAKLLKLKAQLGPDESKQKFVLKTPK

SEQ ID NO: 523 - protein, 41 aa, Homo sapiens
GLKQQKASAELIEEEVAKLLKLKAQLGPDESKQKFVLKTPK

SEQ ID NO: 524 - protein, 40 aa, Homo sapiens
LKQQKASAELIEEEVAKLLKLKAQLGPDESKQKFVLKTPK

SEQ ID NO: 525 - protein, 39 aa, Homo sapiens
KQQKASAELIEEEVAKLLKLKAQLGPDESKQKFVLKTPK

SEQ ID NO: 526 - protein, 38 aa, Homo sapiens
QQKASAELIEEEVAKLLKLKAQLGPDESKQKFVLKTPK

SEQ ID NO: 527 - protein, 37 aa, Homo sapiens
QKASAELIEEEVAKLLKLKAQLGPDESKQKFVLKTPK

SEQ ID NO: 528 - protein, 36 aa, Homo sapiens
KASAELIEEEVAKLLKLKAQLGPDESKQKFVLKTPK

SEQ ID NO: 529 - protein, 35 aa, Homo sapiens
ASAELIEEEVAKLLKLKAQLGPDESKQKFVLKTPK

SEQ ID NO: 530 - protein, 34 aa, Homo sapiens
SAELIEEEVAKLLKLKAQLGPDESKQKFVLKTPK

SEQ ID NO: 531 - protein, 33 aa, Homo sapiens
AELIEEEVAKLLKLKAQLGPDESKQKFVLKTPK

SEQ ID NO: 532 - protein, 32 aa, Homo sapiens
ELIEEEVAKLLKLKAQLGPDESKQKFVLKTPK

SEQ ID NO: 533 - protein, 31 aa, Homo sapiens
LIEEEVAKLLKLKAQLGPDESKQKFVLKTPK

SEQ ID NO: 534 - protein, 30 aa, Homo sapiens
IEEEVAKLLKLKAQLGPDESKQKFVLKTPK

SEQ ID NO: 535 - protein, 29 aa, Homo sapiens
EEEVAKLLKLKAQLGPDESKQKFVLKTPK

SEQ ID NO: 536 - protein, 28 aa, Homo sapiens
EEVAKLLKLKAQLGPDESKQKFVLKTPK

SEQ ID NO: 537 - protein, 27 aa, Homo sapiens
EVAKLLKLKAQLGPDESKQKFVLKTPK

SEQ ID NO: 538 - protein, 26 aa, Homo sapiens
VAKLLKLKAQLGPDESKQKFVLKTPK

SEQ ID NO: 539 - protein, 25 aa, Homo sapiens
AKLLKLKAQLGPDESKQKFVLKTPK

SEQ ID NO: 540 - protein, 24 aa, Homo sapiens
KLLKLKAQLGPDESKQKFVLKTPK

SEQ ID NO: 541 - protein, 23 aa, Homo sapiens
LLKLKAQLGPDESKQKFVLKTPK

SEQ ID NO: 542 - protein, 22 aa, Homo sapiens
LKLKAQLGPDESKQKFVLKTPK

SEQ ID NO: 543 - protein, 21 aa, Homo sapiens
KLKAQLGPDESKQKFVLKTPK

SEQ ID NO: 544 - protein, 41 aa, Homo sapiens
LVKLQGERVRGLKQQKASAELIEEEVAKLLKLKAQLGPDES

SEQ ID NO: 545 - protein, 40 aa, Homo sapiens
VKLQGERVRGLKQQKASAELIEEEVAKLLKLKAQLGPDES

SEQ ID NO: 546 - protein, 39 aa, Homo sapiens
KLQGERVRGLKQQKASAELIEEEVAKLLKLKAQLGPDES

SEQ ID NO: 547 - protein, 38 aa, Homo sapiens
LQGERVRGLKQQKASAELIEEEVAKLLKLKAQLGPDES

SEQ ID NO: 548 - protein, 37 aa, Homo sapiens
QGERVRGLKQQKASAELIEEEVAKLLKLKAQLGPDES

SEQ ID NO: 549 - protein, 36 aa, Homo sapiens
GERVRGLKQQKASAELIEEEVAKLLKLKAQLGPDES

SEQ ID NO: 550 - protein, 35 aa, Homo sapiens
ERVRGLKQQKASAELIEEEVAKLLKLKAQLGPDES

SEQ ID NO: 551 - protein, 34 aa, Homo sapiens
RVRGLKQQKASAELIEEEVAKLLKLKAQLGPDES

SEQ ID NO: 552 - protein, 33 aa, Homo sapiens
VRGLKQQKASAELIEEEVAKLLKLKAQLGPDES

SEQ ID NO: 553 - protein, 32 aa, Homo sapiens
RGLKQQKASAELIEEEVAKLLKLKAQLGPDES

SEQ ID NO: 554 - protein, 31 aa, Homo sapiens
GLKQQKASAELIEEEVAKLLKLKAQLGPDES

SEQ ID NO: 555 - protein, 30 aa, Homo sapiens
LKQQKASAELIEEEVAKLLKLKAQLGPDES

SEQ ID NO: 556 - protein, 29 aa, Homo sapiens
KQQKASAELIEEEVAKLLKLKAQLGPDES

SEQ ID NO: 557 - protein, 28 aa, Homo sapiens
QQKASAELIEEEVAKLLKLKAQLGPDES

SEQ ID NO: 558 - protein, 27 aa, Homo sapiens
QKASAELIEEEVAKLLKLKAQLGPDES

SEQ ID NO: 559 - protein, 26 aa, Homo sapiens
KASAELIEEEVAKLLKLKAQLGPDES

SEQ ID NO: 560 - protein, 25 aa, Homo sapiens
ASAELIEEEVAKLLKLKAQLGPDES

SEQ ID NO: 561 - protein, 24 aa, Homo sapiens
SAELIEEEVAKLLKLKAQLGPDES

SEQ ID NO: 562 - protein, 23 aa, Homo sapiens
AELIEEEVAKLLKLKAQLGPDES

SEQ ID NO: 563 - protein, 22 aa, Homo sapiens
ELIEEEVAKLLKLKAQLGPDES

SEQ ID NO: 564 - protein, 21 aa, Homo sapiens
LIEEEVAKLLKLKAQLGPDES

SEQ ID NO: 565 - protein, 20 aa, Homo sapiens
IEEEVAKLLKLKAQLGPDES

SEQ ID NO: 566 - protein, 19 aa, Homo sapiens
EEEVAKLLKLKAQLGPDES

SEQ ID NO: 567 - protein, 18 aa, Homo sapiens
EEVAKLLKLKAQLGPDES

SEQ ID NO: 568 - protein, 17 aa, Homo sapiens
EVAKLLKLKAQLGPDES

SEQ ID NO: 569 - protein, 16 aa, Homo sapiens
VAKLLKLKAQLGPDES

SEQ ID NO: 570 - protein, 15 aa, Homo sapiens
AKLLKLKAQLGPDES

SEQ ID NO: 571 - protein, 14 aa, Homo sapiens
KLLKLKAQLGPDES

SEQ ID NO: 572 - protein, 13 aa, Homo sapiens
LLKLKAQLGPDES

SEQ ID NO: 573 - protein, 12 aa, Homo sapiens
LKLKAQLGPDES

SEQ ID NO: 574 - protein, 11 aa, Homo sapiens
KLKAQLGPDES

SEQ ID NO: 575 - protein, 31 aa, Homo sapiens
LVKLQGERVRGLKQQKASAELIEEEVAKLLK

SEQ ID NO: 576 - protein, 30 aa, Homo sapiens
VKLQGERVRGLKQQKASAELIEEEVAKLLK

SEQ ID NO: 577 - protein, 29 aa, Homo sapiens
KLQGERVRGLKQQKASAELIEEEVAKLLK

SEQ ID NO: 578 - protein, 28 aa, Homo sapiens
LQGERVRGLKQQKASAELIEEEVAKLLK

SEQ ID NO: 579 - protein, 27 aa, Homo sapiens
QGERVRGLKQQKASAELIEEEVAKLLK

SEQ ID NO: 580 - protein, 26 aa, Homo sapiens
GERVRGLKQQKASAELIEEEVAKLLK

SEQ ID NO: 581 - protein, 25 aa, Homo sapiens
ERVRGLKQQKASAELIEEEVAKLLK

SEQ ID NO: 582 - protein, 24 aa, Homo sapiens
RVRGLKQQKASAELIEEEVAKLLK

SEQ ID NO: 583 - protein, 23 aa, Homo sapiens
VRGLKQQKASAELIEEEVAKLLK

SEQ ID NO: 584 - protein, 22 aa, Homo sapiens
RGLKQQKASAELIEEEVAKLLK

SEQ ID NO: 585 - protein, 21 aa, Homo sapiens
GLKQQKASAELIEEEVAKLLK

SEQ ID NO: 586 - protein, 20 aa, Homo sapiens
LKQQKASAELIEEEVAKLLK

SEQ ID NO: 587 - protein, 19 aa, Homo sapiens
KQQKASAELIEEEVAKLLK

SEQ ID NO: 588 - protein, 18 aa, Homo sapiens
QQKASAELIEEEVAKLLK

SEQ ID NO: 589 - protein, 17 aa, Homo sapiens
QKASAELIEEEVAKLLK

SEQ ID NO: 590 - protein, 16 aa, Homo sapiens
KASAELIEEEVAKLLK

SEQ ID NO: 591 - protein, 15 aa, Homo sapiens
ASAELIEEEVAKLLK

SEQ ID NO: 592 - protein, 14 aa, Homo sapiens
SAELIEEEVAKLLK

SEQ ID NO: 593 - protein, 13 aa, Homo sapiens
AELIEEEVAKLLK

SEQ ID NO: 594 - protein, 12 aa, Homo sapiens
ELIEEEVAKLLK

SEQ ID NO: 595 - protein, 11 aa, Homo sapiens
LIEEEVAKLLK

SEQ ID NO: 596 - protein, 141 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAM

SEQ ID NO: 597 - protein, 408 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTE

SEQ ID NO: 598 - protein, 113 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKL

SEQ ID NO: 599 - protein, 60 aa, Homo sapiens; identical to SEQ ID NO: 443

SEQ ID NO: 600 - protein, 270 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVGYPWWNSCSRILNYPKTSRPWRAWET

SEQ ID NO: 601 - protein, 105 aa, Homo sapiens
RTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 602 - protein, 395 aa, Homo sapiens
MAERAALEELVKLQGERVRGLKQQKASAELIEEEVAKLLKLKAQLGPDESKQKFVLKTPKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 603 - protein, 359 aa, Homo sapiens
MAERAALEELVKLQGERVRGLKQQKASAELIEEEVAKLLKLKAQLGPDESKQKFVLKTPKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 604 - protein, 399 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 605 - protein, 473 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 606 - protein, 469 aa, Homo sapiens
MAERAALEELVKLQGERVRGLKQQKASAELIEEEVAKLLKLKAQLGPDESKQKFVLKTPKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 607 - protein, 435 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 608 - protein, 171 aa, Homo sapiens
MAERAALEELVKLQGERVRGLKQQKASAELIEEEVAKLLKLKAQLGPDESKQKFVLKTPKALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 609 - protein, 211 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 610 - protein, 141 aa, Homo sapiens [sequence omitted]

SEQ ID NO: 611 - protein, 143 aa, Homo sapiens
CLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTG

SEQ ID NO: 612 - protein, 506 aa, Homo sapiens
MPLLGLLPRRAWASLLSQLLRPPCASCTGAVRCQSQVAEAVLTSQLKAHQEKPNFIIKTPKGTRDLSPQHMVVREKILDLVISCFKRHGAKGMDTPAFELKETLTEKYGEDSGLMYDLKDQGGELLSLRYDLTVPFARYLAMNKVKKMKRYHVGKVWRRESPTIVQGRYREFCQCDFDIAGQFDPMIPDAECLKIMCEILSGLQLGDFLIKVNDRRIVDGMFAVCGVPESKFRAICSSIDKLDKMAWKDVRHEMVVKKGLAPEVADRIGDYVQCHGGVSLVEQMFQDPRLSQNKQALEGLGDLKLLFEYLTLFGIADKISFDLSLARGLDYYTGVIYEAVLLQTPTQAGEEPLNVGSVAAGGRYDGLVGMFDPKGHKVPCVGLSIGVERIFYIVEQRMKTKGEKVRTTETQVFVATPQKNFLQERLKLIAELWDSGIKAEMLYKNNPKLLTQLHYCESTGIPLVVIIGEQELKEGVIKIRSVASREEVAIKRENFVAEIQKRLSES

SEQ ID NO: 613 - protein, 247 aa, Homo sapiens
HVGKVWRRESPTIVQGRYREFCQCDFDIAGQFDPMIPDAECLKIMCEILSGLQLGDFLIKVNDRRIVDGMFAVCGVPESKFRAICSSIDKLDKMAWKDVRHEMVVKKGLAPEVADRIGDYVQCHGGVSLVEQMFQDPRLSQNKQALEGLGDLKLLFEYLTLFGIADKISFDLSLARGLDYYTGVIYEAVLLQTPTQAGEEPLNVGSVAAGGRYDGLVGMFDPKGHKVPCVGLSIGVERIFYIVEQRM

SEQ ID NO: 614 - protein, 79 aa, Homo sapiens
QALEGLGDLKLLFEYLTLFGIDDKISFDLSLARGLDYYTGVIYEAVLLQTPAQAGEEPLGVGSVAAGGRYDGLVGMFDP

SEQ ID NO: 615 - protein, 456 aa, Homo sapiens
FVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 616 - protein, 345 aa, Homo sapiens
FVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLE

SEQ ID NO: 617 - protein, 338 aa, Homo sapiens
GTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLE

SEQ ID NO: 618 - protein, 111 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 619 - protein, 96 aa, Homo sapiens
TTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 620 - protein, 102 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKR

SEQ ID NO: 621 - protein, 101 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIK

SEQ ID NO: 622 - protein, 100 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEI

SEQ ID NO: 623 - protein, 99 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVEE

SEQ ID NO: 624 - protein, 98 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLVE

SEQ ID NO: 625 - protein, 97 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDLV

SEQ ID NO: 626 - protein, 96 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRREDL

SEQ ID NO: 627 - protein, 95 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRRED

SEQ ID NO: 628 - protein, 95 aa, Homo sapiens; identical to SEQ ID NO: 627

SEQ ID NO: 629 - protein, 94 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRRE

SEQ ID NO: 630 - protein, 93 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVRR

SEQ ID NO: 631 - protein, 92 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDVR

SEQ ID NO: 632 - protein, 91 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVDV

SEQ ID NO: 633 - protein, 90 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEVD

SEQ ID NO: 634 - protein, 89 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREEV

SEQ ID NO: 635 - protein, 88 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSREE

SEQ ID NO: 636 - protein, 87 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSRE

SEQ ID NO: 637 - protein, 86 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTSR

SEQ ID NO: 638 - protein, 85 aa, Homo sapiens
ALEEKIRTTETQVLVASAQKKLLEERLKLVSELWDAGIKAELLYKKNPKLLNQLQYCEEAGIPLVAIIGEQELKDGVIKLRSVTS

SEQ ID NO: 639 - protein, 84 aa, Homo sapiens
ALEEKIRTTETQVLVASAQKKLLEERLKLVSELWDAGIKAELLYKKNPKLLNQLQYCEEAGIPLVAIIGEQELKDGVIKLRSVT

SEQ ID NO: 640 - protein, 83 aa, Homo sapiens
ALEEKIRTTETQVLVASAQKKLLEERLKLVSELWDAGIKAELLYKKNPKLLNQLQYCEEAGIPLVAIIGEQELKDGVIKLRSV

SEQ ID NO: 641 - protein, 82 aa, Homo sapiens
ALEEKIRTTETQVLVASAQKKLLEERLKLVSELWDAGIKAELLYKKNPKLLNQLQYCEEAGIPLVAIIGEQELKDGVIKLRS

SEQ ID NO: 642 - protein, 81 aa, Homo sapiens
ALEEKIRTTETQVLVASAQKKLLEERLKLVSELWDAGIKAELLYKKNPKLLNQLQYCEEAGIPLVAIIGEQELKDGVIKLR

SEQ ID NO: 643 - protein, 80 aa, Homo sapiens
ALEEKIRTTETQVLVASAQKKLLEERLKLVSELWDAGIKAELLYKKNPKLLNQLQYCEEAGIPLVAIIGEQELKDGVIKL

SEQ ID NO: 644 - protein, 79 aa, Homo sapiens
ALEEKIRTTETQVLVASAQKKLLEERLKLVSELWDAGIKAELLYKKNPKLLNQLQYCEEAGIPLVAIIGEQELKDGVIK

SEQ ID NO: 645 - protein, 78 aa, Homo sapiens
ALEEKIRTTETQVLVASAQKKLLEERLKLVSELWDAGIKAELLYKKNPKLLNQLQYCEEAGIPLVAIIGEQELKDGVI

SEQ ID NO: 646 - protein, 77 aa, Homo sapiens
ALEEKIRTTETQVLVASAQKKLLEERLKLVSELWDAGIKAELLYKKNPKLLNQLQYCEEAGIPLVAIIGEQELKDGV

SEQ ID NO: 647 - protein, 76 aa, Homo sapiens
ALEEKIRTTETQVLVASAQKKLLEERLKLVSELWDAGIKAELLYKKNPKLLNQLQYCEEAGIPLVAIIGEQELKDG

SEQ ID NO: 648 - protein, 75 aa, Homo sapiens
ALEEKIRTTETQVLVASAQKKLLEERLKLVSELWDAGIKAELLYKKNPKLLNQLQYCEEAGIPLVAIIGEQELKD

SEQ ID NO: 649 - protein, 74 aa, Homo sapiens
ALEEKIRTTETQVLVASAQKKLLEERLKLVSELWDAGIKAELLYKKNPKLLNQLQYCEEAGIPLVAIIGEQELK

SEQ ID NO: 650 - protein, 73 aa, Homo sapiens
ALEEKIRTTETQVLVASAQKKLLEERLKLVSELWDAGIKAELLYKKNPKLLNQLQYCEEAGIPLVAIIGEQEL

SEQ ID NO: 651 - protein, 72 aa, Homo sapiens
ALEEKIRTTETQVLVASAQKKLLEERLKLVSELWDAGIKAELLYKKNPKLLNQLQYCEEAGIPLVAIIGEQE

SEQ ID NO: 652 - protein, 71 aa, Homo sapiens
ALEEKIRTTETQVLVASAQKKLLEERLKLVSELWDAGIKAELLYKKNPKLLNQLQYCEEAGIPLVAIIGEQ

SEQ ID NO: 653 - protein, 70 aa, Homo sapiens
ALEEKIRTTETQVLVASAQKKLLEERLKLVSELWDAGIKAELLYKKNPKLLNQLQYCEEAGIPLVAIIGE

SEQ ID NO: 654 - protein, 69 aa, Homo sapiens
ALEEKIRTTETQVLVASAQKKLLEERLKLVSELWDAGIKAELLYKKNPKLLNQLQYCEEAGIPLVAIIG

SEQ ID NO: 655 - protein, 68 aa, Homo sapiens
ALEEKIRTTETQVLVASAQKKLLEERLKLVSELWDAGIKAELLYKKNPKLLNQLQYCEEAGIPLVAII

SEQ ID NO: 656 - protein, 67 aa, Homo sapiens
ALEEKIRTTETQVLVASAQKKLLEERLKLVSELWDAGIKAELLYKKNPKLLNQLQYCEEAGIPLVAI

SEQ ID NO: 657 - protein, 66 aa, Homo sapiens
ALEEKIRTTETQVLVASAQKKLLEERLKLVSELWDAGIKAELLYKKNPKLLNQLQYCEEAGIPLVA

SEQ ID NO: 658 - protein, 65 aa, Homo sapiens
ALEEKIRTTETQVLVASAQKKLLEERLKLVSELWDAGIKAELLYKKNPKLLNQLQYCEEAGIPLV

SEQ ID NO: 659 - protein, 64 aa, Homo sapiens
ALEEKIRTTETQVLVASAQKKLLEERLKLVSELWDAGIKAELLYKKNPKLLNQLQYCEEAGIPL

SEQ ID NO: 660 - protein, 63 aa, Homo sapiens
ALEEKIRTTETQVLVASAQKKLLEERLKLVSELWDAGIKAELLYKKNPKLLNQLQYCEEAGIP

SEQ ID NO: 661 - protein, 62 aa, Homo sapiens
ALEEKIRTTETQVLVASAQKKLLEERLKLVSELWDAGIKAELLYKKNPKLLNQLQYCEEAGI

SEQ ID NO: 662 - protein, 61 aa, Homo sapiens
ALEEKIRTTETQVLVASAQKKLLEERLKLVSELWDAGIKAELLYKKNPKLLNQLQYCEEAG

SEQ ID NO: 663 - protein, 60 aa, Homo sapiens
ALEEKIRTTETQVLVASAQKKLLEERLKLVSELWDAGIKAELLYKKNPKLLNQLQYCEEA

SEQ ID NO: 664 - protein, 59 aa, Homo sapiens
ALEEKIRTTETQVLVASAQKKLLEERLKLVSELWDAGIKAELLYKKNPKLLNQLQYCEE

SEQ ID NO: 665 - protein, 58 aa, Homo sapiens
ALEEKIRTTETQVLVASAQKKLLEERLKLVSELWDAGIKAELLYKKNPKLLNQLQYCE

SEQ ID NO: 666 - protein, 57 aa, Homo sapiens
ALEEKIRTTETQVLVASAQKKLLEERLKLVSELWDAGIKAELLYKKNPKLLNQLQYC

SEQ ID NO: 667 - protein, 56 aa, Homo sapiens
ALEEKIRTTETQVLVASAQKKLLEERLKLVSELWDAGIKAELLYKKNPKLLNQLQY

SEQ ID NO: 668 - protein, 55 aa, Homo sapiens
ALEEKIRTTETQVLVASAQKKLLEERLKLVSELWDAGIKAELLYKKNPKLLNQLQ

SEQ ID NO: 669 - protein, 54 aa, Homo sapiens
ALEEKIRTTETQVLVASAQKKLLEERLKLVSELWDAGIKAELLYKKNPKLLNQL

SEQ ID NO: 670 - protein, 53 aa, Homo sapiens
ALEEKIRTTETQVLVASAQKKLLEERLKLVSELWDAGIKAELLYKKNPKLLNQ

SEQ ID NO: 671 - protein, 52 aa, Homo sapiens
ALEEKIRTTETQVLVASAQKKLLEERLKLVSELWDAGIKAELLYKKNPKLLN

SEQ ID NO: 672 - protein, 51 aa, Homo sapiens
ALEEKIRTTETQVLVASAQKKLLEERLKLVSELWDAGIKAELLYKKNPKLL

SEQ ID NO: 673 - protein, 50 aa, Homo sapiens
ALEEKIRTTETQVLVASAQKKLLEERLKLVSELWDAGIKAELLYKKNPKL

SEQ ID NO: 674 - protein, 49 aa, Homo sapiens
ALEEKIRTTETQVLVASAQKKLLEERLKLVSELWDAGIKAELLYKKNPK

SEQ ID NO: 675 - protein, 48 aa, Homo sapiens
ALEEKIRTTETQVLVASAQKKLLEERLKLVSELWDAGIKAELLYKKNP

SEQ ID NO: 676 - protein, 47 aa, Homo sapiens
ALEEKIRTTETQVLVASAQKKLLEERLKLVSELWDAGIKAELLYKKN

SEQ ID NO: 677 - protein, 46 aa, Homo sapiens
ALEEKIRTTETQVLVASAQKKLLEERLKLVSELWDAGIKAELLYKK

SEQ ID NO: 678 - protein, 45 aa, Homo sapiens
ALEEKIRTTETQVLVASAQKKLLEERLKLVSELWDAGIKAELLYK

SEQ ID NO: 679 - protein, 44 aa, Homo sapiens
ALEEKIRTTETQVLVASAQKKLLEERLKLVSELWDAGIKAELLY

SEQ ID NO: 680 - protein, 43 aa, Homo sapiens
ALEEKIRTTETQVLVASAQKKLLEERLKLVSELWDAGIKAELL

SEQ ID NO: 681 - protein, 42 aa, Homo sapiens
ALEEKIRTTETQVLVASAQKKLLEERLKLVSELWDAGIKAEL

SEQ ID NO: 682 - protein, 41 aa, Homo sapiens
ALEEKIRTTETQVLVASAQKKLLEERLKLVSELWDAGIKAE

SEQ ID NO: 683 - protein, 40 aa, Homo sapiens
ALEEKIRTTETQVLVASAQKKLLEERLKLVSELWDAGIKA

SEQ ID NO: 684 - protein, 39 aa, Homo sapiens
ALEEKIRTTETQVLVASAQKKLLEERLKLVSELWDAGIK

SEQ ID NO: 685 - protein, 38 aa, Homo sapiens
ALEEKIRTTETQVLVASAQKKLLEERLKLVSELWDAGI

SEQ ID NO: 686 - protein, 37 aa, Homo sapiens
ALEEKIRTTETQVLVASAQKKLLEERLKLVSELWDAG

SEQ ID NO: 687 - protein, 36 aa, Homo sapiens
ALEEKIRTTETQVLVASAQKKLLEERLKLVSELWDA

SEQ ID NO: 688 - protein, 35 aa, Homo sapiens
ALEEKIRTTETQVLVASAQKKLLEERLKLVSELWD

SEQ ID NO: 689 - protein, 34 aa, Homo sapiens
ALEEKIRTTETQVLVASAQKKLLEERLKLVSELW

SEQ ID NO: 690 - protein, 33 aa, Homo sapiens
ALEEKIRTTETQVLVASAQKKLLEERLKLVSEL

SEQ ID NO: 691 - protein, 32 aa, Homo sapiens
ALEEKIRTTETQVLVASAQKKLLEERLKLVSE

SEQ ID NO: 692 - protein, 31 aa, Homo sapiens
ALEEKIRTTETQVLVASAQKKLLEERLKLVS

SEQ ID NO: 693 - protein, 30 aa, Homo sapiens
ALEEKIRTTETQVLVASAQKKLLEERLKLV

SEQ ID NO: 694 - protein, 29 aa, Homo sapiens
ALEEKIRTTETQVLVASAQKKLLEERLKL

SEQ ID NO: 695 - protein, 28 aa, Homo sapiens
ALEEKIRTTETQVLVASAQKKLLEERLK

SEQ ID NO: 696 - protein, 27 aa, Homo sapiens
ALEEKIRTTETQVLVASAQKKLLEERL

SEQ ID NO: 697 - protein, 26 aa, Homo sapiens
ALEEKIRTTETQVLVASAQKKLLEER

SEQ ID NO: 698 - protein, 25 aa, Homo sapiens
ALEEKIRTTETQVLVASAQKKLLEE

SEQ ID NO: 699 - protein, 24 aa, Homo sapiens
ALEEKIRTTETQVLVASAQKKLLE

SEQ ID NO: 700 - protein, 23 aa, Homo sapiens
ALEEKIRTTETQVLVASAQKKLL

SEQ ID NO: 701 - protein, 22 aa, Homo sapiens
ALEEKIRTTETQVLVASAQKKL

SEQ ID NO: 702 - protein, 102 aa, Homo sapiens
LEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 703 - protein, 101 aa, Homo sapiens
EEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 704 - protein, 100 aa, Homo sapiens
EKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 705 - protein, 99 aa, Homo sapiens
KIRTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 706 - protein, 97 aa, Homo sapiens
RTTETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 707 - protein, 96 aa, Homo sapiens; identical to SEQ ID NO: 619

SEQ ID NO: 708 - protein, 95 aa, Homo sapiens
TETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 709 - protein, 94 aa, Homo sapiens
ETQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 710 - protein, 93 aa, Homo sapiens
TQVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 711 - protein, 92 aa, Homo sapiens
QVLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 712 - protein, 91 aa, Homo sapiens
VLVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 713 - protein, 90 aa, Homo sapiens
LVASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 714 - protein, 89 aa, Homo sapiens
VASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 715 - protein, 88 aa, Homo sapiens
ASAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 716 - protein, 87 aa, Homo sapiens
SAQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 717 - protein, 86 aa, Homo sapiens
AQKKLLEERLKLVSELWDAGIKAELLYKKNPKLLNQLQYCEEAGIPLVAIIGEQELKDGVIKLRSVTSREEVDVRREDLVEEIKRR

SEQ ID NO: 718 - protein, 85 aa, Homo sapiens
QKKLLEERLKLVSELWDAGIKAELLYKKNPKLLNQLQYCEEAGIPLVAIIGEQELKDGVIKLRSVTSREEVDVRREDLVEEIKRR

SEQ ID NO: 719 - protein, 84 aa, Homo sapiens
KKLLEERLKLVSELWDAGIKAELLYKKNPKLLNQLQYCEEAGIPLVAIIGEQELKDGVIKLRSVTSREEVDVRREDLVEEIKRR

SEQ ID NO: 720 - protein, 83 aa, Homo sapiens
KLLEERLKLVSELWDAGIKAELLYKKNPKLLNQLQYCEEAGIPLVAIIGEQELKDGVIKLRSVTSREEVDVRREDLVEEIKRR

SEQ ID NO: 721 - protein, 82 aa, Homo sapiens
LLEERLKLVSELWDAGIKAELLYKKNPKLLNQLQYCEEAGIPLVAIIGEQELKDGVIKLRSVTSREEVDVRREDLVEEIKRR

SEQ ID NO: 722 - protein, 81 aa, Homo sapiens
LEERLKLVSELWDAGIKAELLYKKNPKLLNQLQYCEEAGIPLVAIIGEQELKDGVIKLRSVTSREEVDVRREDLVEEIKRR

SEQ ID NO: 723 - protein, 80 aa, Homo sapiens
EERLKLVSELWDAGIKAELLYKKNPKLLNQLQYCEEAGIPLVAIIGEQELKDGVIKLRSVTSREEVDVRREDLVEEIKRR

SEQ ID NO: 724 - protein, 79 aa, Homo sapiens
ERLKLVSELWDAGIKAELLYKKNPKLLNQLQYCEEAGIPLVAIIGEQELKDGVIKLRSVTSREEVDVRREDLVEEIKRR

SEQ ID NO: 725 - protein, 78 aa, Homo sapiens
RLKLVSELWDAGIKAELLYKKNPKLLNQLQYCEEAGIPLVAIIGEQELKDGVIKLRSVTSREEVDVRREDLVEEIKRR

SEQ ID NO: 726 - protein, 77 aa, Homo sapiens
LKLVSELWDAGIKAELLYKKNPKLLNQLQYCEEAGIPLVAIIGEQELKDGVIKLRSVTSREEVDVRREDLVEEIKRR

SEQ ID NO: 727 - protein, 76 aa, Homo sapiens
KLVSELWDAGIKAELLYKKNPKLLNQLQYCEEAGIPLVAIIGEQELKDGVIKLRSVTSREEVDVRREDLVEEIKRR

SEQ ID NO: 728 - protein, 75 aa, Homo sapiens
LVSELWDAGIKAELLYKKNPKLLNQLQYCEEAGIPLVAIIGEQELKDGVIKLRSVTSREEVDVRREDLVEEIKRR

SEQ ID NO: 729 - protein, 74 aa, Homo sapiens
VSELWDAGIKAELLYKKNPKLLNQLQYCEEAGIPLVAIIGEQELKDGVIKLRSVTSREEVDVRREDLVEEIKRR

SEQ ID NO: 730 - protein, 73 aa, Homo sapiens
SELWDAGIKAELLYKKNPKLLNQLQYCEEAGIPLVAIIGEQELKDGVIKLRSVTSREEVDVRREDLVEEIKRR

SEQ ID NO: 731 - protein, 72 aa, Homo sapiens
ELWDAGIKAELLYKKNPKLLNQLQYCEEAGIPLVAIIGEQELKDGVIKLRSVTSREEVDVRREDLVEEIKRR

SEQ ID NO: 732 - protein, 71 aa, Homo sapiens
LWDAGIKAELLYKKNPKLLNQLQYCEEAGIPLVAIIGEQELKDGVIKLRSVTSREEVDVRREDLVEEIKRR

SEQ ID NO: 733 - protein, 70 aa, Homo sapiens
WDAGIKAELLYKKNPKLLNQLQYCEEAGIPLVAIIGEQELKDGVIKLRSVTSREEVDVRREDLVEEIKRR

SEQ ID NO: 734 - protein, 69 aa, Homo sapiens
DAGIKAELLYKKNPKLLNQLQYCEEAGIPLVAIIGEQELKDGVIKLRSVTSREEVDVRREDLVEEIKRR

SEQ ID NO: 735 - protein, 68 aa, Homo sapiens
AGIKAELLYKKNPKLLNQLQYCEEAGIPLVAIIGEQELKDGVIKLRSVTSREEVDVRREDLVEEIKRR

SEQ ID NO: 736 - protein, 67 aa, Homo sapiens
GIKAELLYKKNPKLLNQLQYCEEAGIPLVAIIGEQELKDGVIKLRSVTSREEVDVRREDLVEEIKRR

SEQ ID NO: 737 - protein, 66 aa, Homo sapiens
IKAELLYKKNPKLLNQLQYCEEAGIPLVAIIGEQELKDGVIKLRSVTSREEVDVRREDLVEEIKRR

SEQ ID NO: 738 - protein, 65 aa, Homo sapiens
KAELLYKKNPKLLNQLQYCEEAGIPLVAIIGEQELKDGVIKLRSVTSREEVDVRREDLVEEIKRR

SEQ ID NO: 739 - protein, 64 aa, Homo sapiens
AELLYKKNPKLLNQLQYCEEAGIPLVAIIGEQELKDGVIKLRSVTSREEVDVRREDLVEEIKRR

SEQ ID NO: 740 - protein, 63 aa, Homo sapiens
ELLYKKNPKLLNQLQYCEEAGIPLVAIIGEQELKDGVIKLRSVTSREEVDVRREDLVEEIKRR

SEQ ID NO: 741 - protein, 62 aa, Homo sapiens
LLYKKNPKLLNQLQYCEEAGIPLVAIIGEQELKDGVIKLRSVTSREEVDVRREDLVEEIKRR

SEQ ID NO: 742 - protein, 61 aa, Homo sapiens
LYKKNPKLLNQLQYCEEAGIPLVAIIGEQELKDGVIKLRSVTSREEVDVRREDLVEEIKRR

SEQ ID NO: 743 - protein, 60 aa, Homo sapiens
YKKNPKLLNQLQYCEEAGIPLVAIIGEQELKDGVIKLRSVTSREEVDVRREDLVEEIKRR

SEQ ID NO: 744 - protein, 59 aa, Homo sapiens
KKNPKLLNQLQYCEEAGIPLVAIIGEQELKDGVIKLRSVTSREEVDVRREDLVEEIKRR

SEQ ID NO: 745 - protein, 58 aa, Homo sapiens
KNPKLLNQLQYCEEAGIPLVAIIGEQELKDGVIKLRSVTSREEVDVRREDLVEEIKRR

SEQ ID NO: 746 - protein, 57 aa, Homo sapiens
NPKLLNQLQYCEEAGIPLVAIIGEQELKDGVIKLRSVTSREEVDVRREDLVEEIKRR

SEQ ID NO: 747 - protein, 56 aa, Homo sapiens
PKLLNQLQYCEEAGIPLVAIIGEQELKDGVIKLRSVTSREEVDVRREDLVEEIKRR

SEQ ID NO: 748 - protein, 55 aa, Homo sapiens
KLLNQLQYCEEAGIPLVAIIGEQELKDGVIKLRSVTSREEVDVRREDLVEEIKRR

SEQ ID NO: 749 - protein, 54 aa, Homo sapiens
LLNQLQYCEEAGIPLVAIIGEQELKDGVIKLRSVTSREEVDVRREDLVEEIKRR

SEQ ID NO: 750 - protein, 53 aa, Homo sapiens
LNQLQYCEEAGIPLVAIIGEQELKDGVIKLRSVTSREEVDVRREDLVEEIKRR

SEQ ID NO: 751 - protein, 52 aa, Homo sapiens
NQLQYCEEAGIPLVAIIGEQELKDGVIKLRSVTSREEVDVRREDLVEEIKRR

SEQ ID NO: 752 - protein, 51 aa, Homo sapiens
QLQYCEEAGIPLVAIIGEQELKDGVIKLRSVTSREEVDVRREDLVEEIKRR

SEQ ID NO: 753 - protein, 50 aa, Homo sapiens
LQYCEEAGIPLVAIIGEQELKDGVIKLRSVTSREEVDVRREDLVEEIKRR

SEQ ID NO: 754 - protein, 49 aa, Homo sapiens
QYCEEAGIPLVAIIGEQELKDGVIKLRSVTSREEVDVRREDLVEEIKRR

SEQ ID NO: 755 - protein, 48 aa, Homo sapiens
YCEEAGIPLVAIIGEQELKDGVIKLRSVTSREEVDVRREDLVEEIKRR

SEQ ID NO: 756 - protein, 47 aa, Homo sapiens
CEEAGIPLVAIIGEQELKDGVIKLRSVTSREEVDVRREDLVEEIKRR

SEQ ID NO: 757 - protein, 46 aa, Homo sapiens
EEAGIPLVAIIGEQELKDGVIKLRSVTSREEVDVRREDLVEEIKRR

SEQ ID NO: 758 - protein, 45 aa, Homo sapiens
EAGIPLVAIIGEQELKDGVIKLRSVTSREEVDVRREDLVEEIKRR

SEQ ID NO: 759 - protein, 44 aa, Homo sapiens
AGIPLVAIIGEQELKDGVIKLRSVTSREEVDVRREDLVEEIKRR

SEQ ID NO: 760 - protein, 43 aa, Homo sapiens
GIPLVAIIGEQELKDGVIKLRSVTSREEVDVRREDLVEEIKRR

SEQ ID NO: 761 - protein, 42 aa, Homo sapiens
IPLVAIIGEQELKDGVIKLRSVTSREEVDVRREDLVEEIKRR

SEQ ID NO: 762 - protein, 41 aa, Homo sapiens
PLVAIIGEQELKDGVIKLRSVTSREEVDVRREDLVEEIKRR

SEQ ID NO: 763 - protein, 40 aa, Homo sapiens
LVAIIGEQELKDGVIKLRSVTSREEVDVRREDLVEEIKRR

SEQ ID NO: 764 - protein, 39 aa, Homo sapiens
VAIIGEQELKDGVIKLRSVTSREEVDVRREDLVEEIKRR

SEQ ID NO: 765 - protein, 38 aa, Homo sapiens
AIIGEQELKDGVIKLRSVTSREEVDVRREDLVEEIKRR

SEQ ID NO: 766 - protein, 37 aa, Homo sapiens
IIGEQELKDGVIKLRSVTSREEVDVRREDLVEEIKRR

SEQ ID NO: 767 - protein, 36 aa, Homo sapiens
IGEQELKDGVIKLRSVTSREEVDVRREDLVEEIKRR

SEQ ID NO: 768 - protein, 35 aa, Homo sapiens
GEQELKDGVIKLRSVTSREEVDVRREDLVEEIKRR

SEQ ID NO: 769 - protein, 34 aa, Homo sapiens
EQELKDGVIKLRSVTSREEVDVRREDLVEEIKRR

SEQ ID NO: 770 - protein, 33 aa, Homo sapiens
QELKDGVIKLRSVTSREEVDVRREDLVEEIKRR

SEQ ID NO: 771 - protein, 32 aa, Homo sapiens
ELKDGVIKLRSVTSREEVDVRREDLVEEIKRR

SEQ ID NO: 772 - protein, 31 aa, Homo sapiens
LKDGVIKLRSVTSREEVDVRREDLVEEIKRR

SEQ ID NO: 773 - protein, 30 aa, Homo sapiens
KDGVIKLRSVTSREEVDVRREDLVEEIKRR

SEQ ID NO: 774 - protein, 29 aa, Homo sapiens
DGVIKLRSVTSREEVDVRREDLVEEIKRR

SEQ ID NO: 775 - protein, 28 aa, Homo sapiens
GVIKLRSVTSREEVDVRREDLVEEIKRR

SEQ ID NO: 776 - protein, 27 aa, Homo sapiens
VIKLRSVTSREEVDVRREDLVEEIKRR

SEQ ID NO: 777 - protein, 26 aa, Homo sapiens
IKLRSVTSREEVDVRREDLVEEIKRR

SEQ ID NO: 778 - protein, 25 aa, Homo sapiens
KLRSVTSREEVDVRREDLVEEIKRR

SEQ ID NO: 779 - protein, 24 aa, Homo sapiens
LRSVTSREEVDVRREDLVEEIKRR

SEQ ID NO: 780 - protein, 23 aa, Homo sapiens
RSVTSREEVDVRREDLVEEIKRR

SEQ ID NO: 781 - protein, 22 aa, Homo sapiens
SVTSREEVDVRREDLVEEIKRR

SEQ ID NO: 782 - protein, 21 aa, Homo sapiens
VTSREEVDVRREDLVEEIKRR

SEQ ID NO: 783 - protein, 20 aa, Homo sapiens
TSREEVDVRREDLVEEIKRR

SEQ ID NO: 784 - protein, 23 aa, Homo sapiens
LQGERVRGLKQQKASAELIEEEV

SEQ ID NO: 785 - protein, 8 aa, Homo sapiens
KFVLKTPK

SEQ ID NO: 786 - protein, 16 aa, Homo sapiens
SSVDKLDKVGYPWWNS

SEQ ID NO: 787 - protein, 16 aa, Homo sapiens
KFVLKTPKDFDIAGNF

SEQ ID NO: 788 - protein, 16 aa, Homo sapiens
KFVLKTPKVNDRRILD

SEQ ID NO: 789 - protein, 16 aa, Homo sapiens
DTPVFELKVNDRRILD

SEQ ID NO: 790 - protein, 16 aa, Homo sapiens
RYREFYQCVNDRRILD

SEQ ID NO: 791 - protein, 16 aa, Homo sapiens
KFVLKTPKETLMGKYG

SEQ ID NO: 792 - protein, 16 aa, Homo sapiens
DTPVFELKDFDIAGNF

SEQ ID NO: 793 - protein, 16 aa, Homo sapiens
KFVLKTPKALEEKIRT

SEQ ID NO: 794 - protein, 16 aa, Homo sapiens
DTPVFELKALEEKIRT

SEQ ID NO: 795 - protein, 138 aa, Artificial Sequence
  Consensus HRS WHEP sequence
  MOD_RES (1)..(50): Xaa is any amino acid or absent
  misc_feature (52)..(56): Xaa can be any naturally occurring amino acid
  MOD_RES (57)..(58): Xaa is any amino acid or absent
  MOD_RES (61)..(62): Xaa is any amino acid
  MOD_RES (65)..(65): Xaa is any amino acid
  MOD_RES (68)..(69): Xaa is any amino acid
  misc_feature (72)..(78): Xaa can be any naturally occurring amino acid
  MOD_RES (79)..(80): Xaa is any amino acid or absent
  MOD_RES (82)..(83): Xaa is any amino acid
  MOD_RES (86)..(86): Xaa is any amino acid
  MOD_RES (89)..(138): Xaa is any amino acid or absent
XXXXXXXXXXXXXXXXXXXXXXXXXXXXXXXXXXXXXXXXXXXXXXXXXXLXXXXXXXQGXXVRXLKXXKAXXXXXXXXXVXXLLXLKXXXXXXXXXXXXXXXXXXXXXXXXXXXXXXXXXXXXXXXXXXXXXXXXXX

SEQ ID NO: 796 - protein, 904 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYTLDPILITIIAMSSLGVLLGATCAGLLLYCTCSYSGLSSRSCTTLENYNFELYDGLKHKVKMNHQKCCSEA

SEQ ID NO: 797 - protein, 879 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAGGTLLPGTEPTVDTVPMQPIPAYWYYVMAAGGAVLVLVSVALALVLHYHRFRYAAKKTDHSITYKTSHYTNGAPLAVEPTLTIKLEQDRGSHC

SEQ ID NO: 798 - protein, 533 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKVGCSWRPL

SEQ ID NO: 799 - protein, 114 aa, Homo sapiens
CGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEI

SEQ ID NO: 800 - protein, 117 aa, Homo sapiens
CSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYY

SEQ ID NO: 801 - protein, 147 aa, Homo sapiens
PLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFG

SEQ ID NO: 802 - protein, 154 aa, Homo sapiens
LGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLG

SEQ ID NO: 803 - protein, 154 aa, Homo sapiens
PSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTD

SEQ ID NO: 804 - protein, 573 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 805 - protein, 451 aa, Homo sapiens
GSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 806 - protein, 320 aa, Homo sapiens
QCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWT

SEQ ID NO: 807 - protein, 1060 aa, Homo sapiens
QPDPPCGGRLNSKDAGYITSPGYPQDYPSHQNCEWIVYAPEPNQKIVLNFNPHFEIEKHDCKYDFIEIRDGDSESADLLGKHCGNIAPPTIISSGSMLYIKFTSDYARQGAGFSLRYEIFKTGSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDSKPTVETLGPTVKSEETTTPYPTEEEATECGENCSFEDDKDLQLPSGFNCNFDFLEEPCGWMYDHAKWLRTTWASSSSPNDRTFPDDRNFLRLQSDSQREGQYARLISPPVHLPRSPVCMEFQYQATGGRGVALQVVREASQESKLLWVIREDQGGEWKHGRIILPSYDMEYQIVFEGVIGKGRSGEIAIDDIRISTDVPLENCMEPISAFAVDIPEIHEREGYEDEIDDEYEVDWSNSSSATSGSGAPSTDKEKSWLYDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 808 - protein, 678 aa, Homo sapiens
GSEDCSKNFTSPNGTIESPGFPEKYPHNLDCTFTILAKPKMEIILQFLIFDLEHDPLQVGEGDCKYDWLDIWDGIPHVGPLIGKYCGTKTPSELRSSTGILSLTFHTDMAVAKDGFSARYYLVHQEPLENFQCNVPLGMESGRIANEQISASSTYSDGRWTPQQSRLHGDDNGWTPNLDSNKEYLQVDLRFLTMLTAIATQGAISRETQNGYYVKSYKLEVSTNGEDWMVYRHGKNHKVFQANNDATEVVLNKLHAPLLTRFVRIRPQTWHSGIALRLELFGCRVTDAPCSNMLGMLSGLIADSQISASSTQEYLWSPSAARLVSSRSGWFPRIPQAQPGEEWLQVDLGTPKTVKGVIIQGARGGDSITAVEARAFVRKFKVSYSLNGKDWEYIQDPRTQQPKLFEGNMHYDTPDIRRFDPIPAQYVRVYPERWSPAGIGMRLEVLGCDWTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 809 - protein, 509 aa, Mus musculus
MADRAALEELVRLQGAHVRGLKEQKASAEQIEEEVTKLLKLKAQLGQDEGKQKFVLKTPKGTRDYSPRQMAVREKVFDVIIRCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLKIMCEILSSLQIGNFLVKVNDRRILDGMFAVCGVPDSKFRTICSSVDKLDKVSWEEVKNEMVGEKGLAPEVADRIGDYVQQHGGVSLVEQLLQDPKLSQNKQAVEGLGDLKLLFEYLILFGIDDKISFDLSLARGLDYYTGVIYEAVLLQMPTQAGEEPLGVGSIAAGGRYDGLVGMFDPKGRKVPCVGLSIGVERIFSIVEQRLEASEEKVRTTETQVLVASAQKKLLEERLKLVSELWDAGIKAELLYKKNPKLLNQLQYWEEAGIPLVAIIGEQELRDGVIKLRSVASREEVDVRREDLVEEIRRRTNQPLSTC

SEQ ID NO: 810 - protein, 509 aa, Canis lupus familiaris
MAERAALEELVRLQGERVRGLKQQKASAEQIEEEVAKLLKLKAQLGPDEGKQKFVLKTPKGTRDYSPRQMAVREKVFDVIISCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLEIMCEILRSLQIGDFLVKVNDRRILDGMFAICGVPDSKFRTICSSVDKLDKVSWEEVKNEMVGEKGLAPEVADHIGDYVQQHGGISLVEQLLQDPELSQNKQALEGLGDLKLLFEYLTLFGIADKISFDLSLARGLDYYTGVIYEAVLLQTPVQAGEEPLGVGSVAAGGRYDGLVGMFDPKGRKVPCVGLSIGVERIFSIVEQRLEATEEKVRTTETQVLVASAQKKLLEERLKLVSELWNAGIKAELLYKKNPKLLNQLQYCEEAGIPLVAIIGEQELKDGVIKLRSVASREEVDVPREDLVEEIKRRTSQPFCIC

SEQ ID NO: 811 - protein, 509 aa, Bos taurus
MADRAALEDLVRVQGERVRGLKQQKASAEQIEEEVAKLLKLKAQLGPDEGKPKFVLKTPKGTRDYSPRQMAVREKVFDVIISCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMLPDAECLKIMCEILSSLQIGDFLVKVNDRRILDGMFAICGVPDSKFRTICSSVDKLDKVSWEEVKNEMVGEKGLAPEVADRIGDYVQQHGGVSLVEQLLQDPKLSQNKQALEGLGDLKLLFEYLTLFGIADKISFDLSLARGLDYYTGVIYEAVLLQPPARAGEEPLGVGSVAAGGRYDGLVGMFDPKGRKVPCVGLSIGVERIFSIVEQRLEALEEKVRTTETQVLVASAQKKLLEERLKLISELWDAGIKAELLYKKNPKLLNQLQYCEETGIPLVAIIGEQELKDGVIKLRSVASREEVDVRREDLVEEIKRRTSQPLCIC

SEQ ID NO: 812 - protein, 508 aa, Rattus norvegicus
MADRAALEELVRLQGAHVRGLKEQKASAEQIEEEVTKLLKLKAQLGHDEGKQKFVLKTPKGTRDYSPRQMAVREKVFDVIIRCFKRHGAEVIDTPVFELKETLTGKYGEDSKLIYDLKDQGGELLSLRYDLTVPFARYLAMNKLTNIKRYHIAKVYRRDNPAMTRGRYREFYQCDFDIAGQFDPMIPDAECLKIMCEILSSLQIGNFQVKVNDRRILDGMFAVCGVPDSKFRTICSSVDKLDKVSWEEVKNEMVGEKGLAPEVADRIGDYVQQHGGVSLVEQLLQDPKLSQNKQAVEGLGDLKLLFEYLTLFGIDDKISFDLSLARGLDYYTGVIYEAVLLQMPTQAGEEPLGVGSIAAGGRYDGLVGMFDPKGRKVPCVGLSIGVERIFSIVEQKLEASEEKVRTTETQVLVASAQKKLLEERLKLISELWDAGIKAELLYKKNPKLLNQLQYCEEAGIPLVAIIGEQELKDGVIKLRSVTSREEVDVRREDLVEEIRRRTSQPLSM

SEQ ID NO: 813 - protein, 500 aa, Gallus gallus
MADEAAVRQQAEVVRRLKQDKAEPDEIAKEVAKLLEMKAHLGGDEGKHKFVLKTPKGTRDYGPKQMAIRERVFSAIIACFKRHGAEVIDTPVFELKETLTGKYGEDSKLIYDLKDQGGELLSLRYDLTVPFARYLAMNKITNIKRYHIAKVYRRDNPAMTRGRYREFYQCDFDIAGQFDPMIPDAECLKIVQEILSDLQLGDFLIKVNDRRILDGMFAVCGVPDSKFRTICSSVDKLDKMPWEEVRNEMVGEKGLSPEAADRIGEYVQLHGGMDLIEQLLQDPKLSQNKLVKEGLGDMKLLFEYLTLFGITGKISFDLSLARGLDYYTGVIYEAVLLQQNDHGEESVSVGSVAGGGRYDGLVGMFDPKGRKVPCVGISIGIERIFSILEQRVEASEEKIRTTETQVLVASAQKKLLEERLKLISELWDAGIKAEVLYKKNPKLLNQLQYCEDTGIPLVAIVGEQELKDGVVKLRVVATGEEVNIRRESLVEEIRRRTNQL

SEQ ID NO: 814 - protein, 437 aa, Danio rerio
MAALGLVSMRLCAGLMGRRSAVRLHSLRVCSGMTISQIDEEVARLLQLKAQLGGDEGKHVFVLKTAKGTRDYNPKQMAIREKVFNIIINCFKRHGAETIDSPVFELKETLTGKYGEDSKLIYDLKDQGGELLSLRYDLTVPFARYLAMNKITNIKRYHIAKVYRRDNPAMTRGRYREFYQCDFDIAGQYDAMIPDAECLKLVYEILSELDLGDFRIKVNDRRILDGMFAICGVPDEKFRTICSTVDKLDKLAWEEVKKEMVNEKGLSEEVADRIRDYVSMQGGKDLAERLLQDPKLSQSKQACAGITDMKLLFSYLELFQITDKVVFDLSLARGLDYYTGVIYEAILTQANPAPASTPAEQNGAEDAGVSVGSVAGGGRYDGLVGMFDPKAGKCPVWGSALALRGSSPSWSRRQSCLQRRCAPLKLKCLWLQHRRTF

SEQ ID NO: 815 - protein, 435 aa, Macaca fascicularis
MAERAALEELVKLQGERVRGLKQQQASAELIEEEVGKLLKLKAQLGPDESKQKFVLKTPKGTRDYSPRQMAVREKVFDVIIRCFKRHGAEVIDTPVFELKDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKVRTTETQVLVASAQKKLLEERLKLVSELWDAGIKAELLYKKNPKLLNQLQYCEEAGIPLVAIIGEQELKDGVIKLRSVTSREEVNVRREDLVEEIKRRTGQLLRIC

SEQ ID NO: 816 - protein, 509 aa, Macaca mulatta
MAERAALEELVKLQGERVRGLKQQKASAELIEEEVGKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKVRTTETQVLVASAQKKLLEERLKLVSELWDAGIKAELLYKKNPKLLNQLQYCEEAGIPLVAIIGEQELKDGVIKLRSVTSREEVNVRREDLVEEIKRRTGQPLRIC